(19) United States
(12) Patent Application Publication
(10) Pub. No.: US 2025/0163395 A1
(43) Pub. Date: May 22, 2025

(54) SYSTEMS AND METHODS OF NOVEL CRISPR-CAS PROTEINS FOR IN-VITRO APPLICATIONS

(71) Applicant: Roche Molecular Systems, Inc., Pleasanton, CA (US)

(72) Inventors: He Wang, Pleasanton, CA (US); Yu Tian, Pleasanton, CA (US); Jinshuang Lu, Pleasanton, CA (US); Beiyang Ma, Pleasanton, CA (US); Yan Wei, Pleasanton, CA (US); Xiang Li, Pleasanton, CA (US); Renjian Huang, Pleasanton, CA (US); Fangnian Wang, Pleasanton, CA (US)

(73) Assignee: Roche Molecular Systems, Inc., Pleasanton, CA (US)

(21) Appl. No.: 18/955,601

(22) Filed: Nov. 21, 2024

Related U.S. Application Data
(60) Provisional application No. 63/601,691, filed on Nov. 21, 2023.

(51) Int. Cl. C12N 9/22; C12N 15/11; C12N 15/70; C12Q 1/70

(52) U.S. Cl. CPC C12N 9/22; C12N 15/111; C12N 15/70; C12Q 1/70; C07K 2319/21; C12N 2310/20

(57) ABSTRACT

The present disclosure relates to novel CRISPR enzymes, reaction mixtures, methods of using the same in the detection of microorganisms, such as FluA, FluB, amongst others. The disclosure particularly relates to the discovery of novel functional CRISPR enzyme complexes, particularly systems displaying trans cleavage activity.

### RELATED APPLICATIONS

This application claims priority under 35 U.S.C. 119 (e) to U.S. Provisional Patent Application Ser. No. 63/601,691, filed 21 Nov. 2023 and titled “Systems and Methods of Novel CRISPR-CAS Proteins for in-vitro applications,” the disclosure of which is hereby incorporated by reference in its entirety.

### REFERENCE TO A SEQUENCE LISTING SUBMITTED AS A TEXT FILE

The official copy of the sequence listing is submitted electronically via EFS-Web with a file named, “08485-008WO1.xml,” created on Nov. 15, 2024, and having a size of 802 KB kilobytes, and is filed concurrently with the specification. The sequence listing contained in the XML formatted document is part of the specification and is herein incorporated by reference in its entirety.

### FIELD

The following discussion is merely provided to aid the reader in understanding the disclosure and is not admitted to describe or constitute prior art thereto.

Comprehending the connection between an enzyme's amino acid composition and its overall purpose remains highly relevant in practical, clinical, and scientific realms for both biologists and engineers. This has become of particular interest in the CRISPR field, where a number of functional CRISPR enzymes have been identified, while others with similar structure appear non-functional. Although directed evolution is a potent instrument for engineering and exploration, the haphazard and frequently combined nature of mutations poses a challenge in assessing their specific effects, complicating further engineering efforts. A more methodical rapid and high throughput workflow may be required when attempting to simultaneously identify functional activity in multiple putative enzyme sequences. This is particular important if the high throughput screen is designed to identify enzymes with very specific activities, or enzymes that only display functional activity in multi-component complexes, including complexes that include CRISPR enzymes and guide nucleic acids.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

Disclosed herein are reaction mixture(s) comprising: a nucleic acid-guided nuclease (CRISPR nuclease) having at least 85% amino acid homology to any one of SEQ ID NOs:1-93; a guide nucleic acid (gRNA) for complexing with the nucleic acid-guided CRISPR nuclease, wherein the gRNA comprises a nucleic acid sequence (“the gRNA sequence”), and wherein the gRNA sequence comprises a direct repeat (e.g., a nucleic acid sequence) having at least 85% nucleic acid homology to any one of SEQ ID NOs:374-466, optionally wherein the gRNA comprises a nucleic acid sequence having at least 85% nucleic acid homology to any one of SEQ ID NOs:94-279, and wherein the gRNA forms a complex with the CRISPR nuclease in the reaction mixture; and a substrate for the complex. In some instances, the nucleic acid-guided CRISPR nuclease has at least 90%, at least 95%, or at least 99% amino acid homology to any one of SEQ ID NOs:1-93. Also disclosed herein are enzyme systems comprising: a nucleic acid-guided CRISPR nuclease having at least 85% amino acid homology to any one of SEQ ID NOs:1-93; and a guide nucleic acid (gRNA) for complexing with the nucleic acid-guided CRISPR nuclease, wherein the gRNA comprises a nucleic acid sequence comprising a direct repeat having at least 85% nucleic acid homology to any one of SEQ ID NOs:374-466, optionally wherein the gRNA comprises a nucleic acid sequence having at least 85% nucleic acid homology to any one of SEQ ID NOs:94-279. In some instances, the reaction mixtures described herein comprise at least one nucleic acid-guided CRISPR nuclease and a direct repeat sequence the “activates” the CRISPR nuclease, when in the presence of a target sequence. Such functional systems can comprise a nucleic acid-guided CRISPR nuclease/gRNA comprising a direct repeat sequence selected from the group consisting of: the CRISPR nuclease/direct repeat pair of SEQ ID NO:22/SEQ ID NO: 395; the CRISPR nuclease/direct repeat pair of SEQ ID NO:62/SEQ ID NO: 435; the CRISPR nuclease/direct repeat pair of SEQ ID NO:80/SEQ ID NO: 453; and/or the CRISPR nuclease/direct repeat pair of SEQ ID NO:91/SEQ ID NO: 464.

In some instances, the reaction mixture(s) and/or enzyme systems described herein comprise certain CRISPR nuclease/gRNA pairs. In such instances, the homology of the enzyme can be at least 90%, at least 95%, or at least 99% amino acid homology to any one of SEQ ID NOs:1-93. In some instances the nucleic acid-guided CRISPR nuclease and the guide nucleic acid comprise a CRISPR nuclease/gRNA pair selected from the group consisting of: the CRISPR nuclease/gRNA pair of SEQ ID NO:1/SEQ ID NO: 94 or the CRISPR nuclease/gRNA pair of SEQ ID NO:1/SEQ ID NO: 117; the CRISPR nuclease/gRNA pair of SEQ ID NO:2/SEQ ID NO: 95 or the CRISPR nuclease/gRNA pair of SEQ ID NO:2/SEQ ID NO: 118; the CRISPR nuclease/gRNA pair of SEQ ID NO:3/SEQ ID NO: 96 or the CRISPR nuclease/gRNA pair of SEQ ID NO:3/SEQ ID NO: 119; the CRISPR nuclease/gRNA pair of SEQ ID NO:4/SEQ ID NO: 97 or the CRISPR nuclease/gRNA pair of SEQ ID NO:4/SEQ ID NO: 120; the CRISPR nuclease/gRNA pair of SEQ ID NO:5/SEQ ID NO: 98 or the CRISPR nuclease/gRNA pair of SEQ ID NO:5/SEQ ID NO: 121; the CRISPR nuclease/gRNA pair of SEQ ID NO:6/SEQ ID NO: 99 or the CRISPR nuclease/gRNA pair of SEQ ID NO:6/SEQ ID NO: 122; the CRISPR nuclease/gRNA pair of SEQ ID NO:7/SEQ ID NO: 100 or the CRISPR nuclease/gRNA pair of SEQ ID NO:7/SEQ ID NO: 123; the CRISPR nuclease/gRNA pair of SEQ ID NO:8/SEQ ID NO: 101 or the CRISPR nuclease/gRNA pair of SEQ ID NO:8/SEQ ID NO: 124; the CRISPR nuclease/gRNA pair of SEQ ID NO:9/SEQ ID NO: 102 or the CRISPR nuclease/gRNA pair of SEQ ID NO:9/SEQ ID NO: 125; the CRISPR nuclease/gRNA pair of SEQ ID NO:10/SEQ ID NO: 103 or the CRISPR nuclease/gRNA pair of SEQ ID NO:10/SEQ ID NO: 126; the CRISPR nuclease/gRNA pair of SEQ ID NO: 11/SEQ ID NO: 104 or the CRISPR nuclease/gRNA pair of SEQ ID NO: 11/SEQ ID NO: 127; the CRISPR nuclease/gRNA pair of SEQ ID NO: 12/SEQ ID NO: 105 or the CRISPR nuclease/gRNA pair of SEQ ID NO:12/SEQ ID NO: 128; the CRISPR nuclease/gRNA pair of SEQ ID NO:13/SEQ ID NO: 106 or the CRISPR nuclease/gRNA pair of SEQ ID NO: 13/SEQ ID NO: 129; the CRISPR nuclease/gRNA pair of SEQ ID NO: 14/SEQ ID NO: 107 or the CRISPR nuclease/gRNA pair of SEQ ID NO: 14/SEQ ID NO: 130; the CRISPR nuclease/gRNA pair of SEQ ID NO: 15/SEQ ID NO: 108 or the CRISPR nuclease/gRNA pair of SEQ ID NO: 15/SEQ ID NO: 131; the CRISPR nuclease/gRNA pair of SEQ ID NO: 16/SEQ ID NO: 109 or the CRISPR nuclease/gRNA pair of SEQ ID NO: 16/SEQ ID NO: 132; the CRISPR nuclease/gRNA pair of SEQ ID NO: 17/SEQ ID NO: 110 or the CRISPR nuclease/gRNA pair of SEQ ID NO:17/SEQ ID NO: 133; the CRISPR nuclease/gRNA pair of SEQ ID NO: 18/SEQ ID NO: 111 or the CRISPR nuclease/gRNA pair of SEQ ID NO: 18/SEQ ID NO: 134; the CRISPR nuclease/gRNA pair of SEQ ID NO: 19/SEQ ID NO: 112 or the CRISPR nuclease/gRNA pair of SEQ ID NO:19/SEQ ID NO: 135; the CRISPR nuclease/gRNA pair of SEQ ID NO:20/SEQ ID NO: 113 or the CRISPR nuclease/gRNA pair of SEQ ID NO:20/SEQ ID NO: 136; the CRISPR nuclease/gRNA pair of SEQ ID NO:21/SEQ ID NO: 114 or the CRISPR nuclease/gRNA pair of SEQ ID NO:21/SEQ ID NO: 137; the CRISPR nuclease/gRNA pair of SEQ ID NO:22/SEQ ID NO: 115 or the CRISPR nuclease/gRNA pair of SEQ ID NO:22/SEQ ID NO: 138; the CRISPR nuclease/gRNA pair of SEQ ID NO:23/SEQ ID NO: 116 or the CRISPR nuclease/gRNA pair of SEQ ID NO:23/SEQ ID NO: 139; the CRISPR nuclease/gRNA pair of SEQ ID NO:24/SEQ ID NO: 140 or the CRISPR nuclease/gRNA pair of SEQ ID NO:24/SEQ ID NO: 164; the CRISPR nuclease/gRNA pair of SEQ ID NO:25/SEQ ID NO: 141 or the CRISPR nuclease/gRNA pair of SEQ ID NO:25/SEQ ID NO: 165; the CRISPR nuclease/gRNA pair of SEQ ID NO:26/SEQ ID NO: 142 or the CRISPR nuclease/gRNA pair of SEQ ID NO:26/SEQ ID NO: 166; the CRISPR nuclease/gRNA pair of SEQ ID NO:27/SEQ ID NO: 143 or the CRISPR nuclease/gRNA pair of SEQ ID NO:27/SEQ ID NO: 167; the CRISPR nuclease/gRNA pair of SEQ ID NO:28/SEQ ID NO: 144 or the CRISPR nuclease/gRNA pair of SEQ ID NO:28/SEQ ID NO: 168; the CRISPR nuclease/gRNA pair of SEQ ID NO:29/SEQ ID NO: 145 or the CRISPR nuclease/gRNA pair of SEQ ID NO:29/SEQ ID NO: 169; the CRISPR nuclease/gRNA pair of SEQ ID NO:30/SEQ ID NO: 146 or the CRISPR nuclease/gRNA pair of SEQ ID NO:30/SEQ ID NO: 170; the CRISPR nuclease/gRNA pair of SEQ ID NO:31/SEQ ID NO: 147 or the CRISPR nuclease/gRNA pair of SEQ ID NO:31/SEQ ID NO: 171; the CRISPR nuclease/gRNA pair of SEQ ID NO:32/SEQ ID NO: 148 or the CRISPR nuclease/gRNA pair of SEQ ID NO:32/SEQ ID NO: 172; the CRISPR nuclease/gRNA pair of SEQ ID NO:33/SEQ ID NO: 149 or the CRISPR nuclease/gRNA pair of SEQ ID NO:33/SEQ ID NO: 173; the CRISPR nuclease/gRNA pair of SEQ ID NO:34/SEQ ID NO: 150 or the CRISPR nuclease/gRNA pair of SEQ ID NO:34/SEQ ID NO: 174; the CRISPR nuclease/gRNA pair of SEQ ID NO:35/SEQ ID NO: 151 or the CRISPR nuclease/gRNA pair of SEQ ID NO:35/SEQ ID NO: 175; the CRISPR nuclease/gRNA pair of SEQ ID NO:36/SEQ ID NO: 152 or the CRISPR nuclease/gRNA pair of SEQ ID NO:36/SEQ ID NO: 176; the CRISPR nuclease/gRNA pair of SEQ ID NO:37/SEQ ID NO: 153 or the CRISPR nuclease/gRNA pair of SEQ ID NO:37/SEQ ID NO: 177; the CRISPR nuclease/gRNA pair of SEQ ID NO:38/SEQ ID NO: 154 or the CRISPR nuclease/gRNA pair of SEQ ID NO:38/SEQ ID NO: 178; the CRISPR nuclease/gRNA pair of SEQ ID NO:39/SEQ ID NO: 155 or the CRISPR nuclease/gRNA pair of SEQ ID NO:39/SEQ ID NO: 179; the CRISPR nuclease/gRNA pair of SEQ ID NO:40/SEQ ID NO: 156 or the CRISPR nuclease/gRNA pair of SEQ ID NO:40/SEQ ID NO: 180; the CRISPR nuclease/gRNA pair of SEQ ID NO:41/SEQ ID NO: 157 or the CRISPR nuclease/gRNA pair of SEQ ID NO:41/SEQ ID NO: 181; the CRISPR nuclease/gRNA pair of SEQ ID NO:42/SEQ ID NO: 158 or the CRISPR nuclease/gRNA pair of SEQ ID NO:42/SEQ ID NO: 182; the CRISPR nuclease/gRNA pair of SEQ ID NO:43/SEQ ID NO: 159 or the CRISPR nuclease/gRNA pair of SEQ ID NO:43/SEQ ID NO: 183; the CRISPR nuclease/gRNA pair of SEQ ID NO:44/SEQ ID NO: 160 or the CRISPR nuclease/gRNA pair of SEQ ID NO:44/SEQ ID NO: 184; the CRISPR nuclease/gRNA pair of SEQ ID NO:45/SEQ ID NO: 161 or the CRISPR nuclease/gRNA pair of SEQ ID NO:45/SEQ ID NO: 185; the CRISPR nuclease/gRNA pair of SEQ ID NO:46/SEQ ID NO: 162 or the CRISPR nuclease/gRNA pair of SEQ ID NO:46/SEQ ID NO: 186; the CRISPR nuclease/gRNA pair of SEQ ID NO:47/SEQ ID NO: 163 or the CRISPR nuclease/gRNA pair of SEQ ID NO:47/SEQ ID NO: 187; the CRISPR nuclease/gRNA pair of SEQ ID NO:48/SEQ ID NO: 188 or the CRISPR nuclease/gRNA pair of SEQ ID NO:48/SEQ ID NO: 234; the CRISPR nuclease/gRNA pair of SEQ ID NO:49/SEQ ID NO: 189 or the CRISPR nuclease/gRNA pair of SEQ ID NO:49/SEQ ID NO: 235; the CRISPR nuclease/gRNA pair of SEQ ID NO:50/SEQ ID NO: 190 or the CRISPR nuclease/gRNA pair of SEQ ID NO:50/SEQ ID NO: 236; the CRISPR nuclease/gRNA pair of SEQ ID NO:51/SEQ ID NO: 191 or the CRISPR nuclease/gRNA pair of SEQ ID NO:51/SEQ ID NO: 237; the CRISPR nuclease/gRNA pair of SEQ ID NO:52/SEQ ID NO: 192 or the CRISPR nuclease/gRNA pair of SEQ ID NO:52/SEQ ID NO: 238; the CRISPR nuclease/gRNA pair of SEQ ID NO:53/SEQ ID NO: 193 or the CRISPR nuclease/gRNA pair of SEQ ID NO:53/SEQ ID NO: 239; the CRISPR nuclease/gRNA pair of SEQ ID NO:54/SEQ ID NO: 194 or the CRISPR nuclease/gRNA pair of SEQ ID NO:54/SEQ ID NO: 240; the CRISPR nuclease/gRNA pair of SEQ ID NO:55/SEQ ID NO: 195 or the CRISPR nuclease/gRNA pair of SEQ ID NO:55/SEQ ID NO: 241; the CRISPR nuclease/gRNA pair of SEQ ID NO:56/SEQ ID NO: 196 or the CRISPR nuclease/gRNA pair of SEQ ID NO:56/SEQ ID NO: 242; the CRISPR nuclease/gRNA pair of SEQ ID NO:57/SEQ ID NO: 197 or the CRISPR nuclease/gRNA pair of SEQ ID NO:57/SEQ ID NO: 243; the CRISPR nuclease/gRNA pair of SEQ ID NO:58/SEQ ID NO: 198 or the CRISPR nuclease/gRNA pair of SEQ ID NO:58/SEQ ID NO: 244; the CRISPR nuclease/gRNA pair of SEQ ID NO:59/SEQ ID NO: 199 or the CRISPR nuclease/gRNA pair of SEQ ID NO:59/SEQ ID NO: 245; the CRISPR nuclease/gRNA pair of SEQ ID NO:60/SEQ ID NO: 200 or the CRISPR nuclease/gRNA pair of SEQ ID NO:60/SEQ ID NO: 246; the CRISPR nuclease/gRNA pair of SEQ ID NO:61/SEQ ID NO: 201 or the CRISPR nuclease/gRNA pair of SEQ ID NO:61/SEQ ID NO: 247; the CRISPR nuclease/gRNA pair of SEQ ID NO:62/SEQ ID NO: 202 or the CRISPR nuclease/gRNA pair of SEQ ID NO:62/SEQ ID NO: 248; the CRISPR nuclease/gRNA pair of SEQ ID NO:63/SEQ ID NO: 203 or the CRISPR nuclease/gRNA pair of SEQ ID NO:63/SEQ ID NO: 249; the CRISPR nuclease/gRNA pair of SEQ ID NO:64/SEQ ID NO: 204 or the CRISPR nuclease/gRNA pair of SEQ ID NO:64/SEQ ID NO: 250; the CRISPR nuclease/gRNA pair of SEQ ID NO:65/SEQ ID NO: 205 or the CRISPR nuclease/gRNA pair of SEQ ID NO:65/SEQ ID NO: 251; the CRISPR nuclease/gRNA pair of SEQ ID NO:66/SEQ ID NO: 206 or the CRISPR nuclease/gRNA pair of SEQ ID NO:66/SEQ ID NO: 252; the CRISPR nuclease/gRNA pair of SEQ ID NO:67/SEQ ID NO: 207 or the CRISPR nuclease/gRNA pair of SEQ ID NO:67/SEQ ID NO: 253; the CRISPR nuclease/gRNA pair of SEQ ID NO:68/SEQ ID NO: 208 or the CRISPR nuclease/gRNA pair of SEQ ID NO:68/SEQ ID NO: 254; the CRISPR nuclease/gRNA pair of SEQ ID NO:69/SEQ ID NO: 209 or the CRISPR nuclease/gRNA pair of SEQ ID NO:69/SEQ ID NO: 255; the CRISPR nuclease/gRNA pair of SEQ ID NO:70/SEQ ID NO: 210 or the CRISPR nuclease/gRNA pair of SEQ ID NO:70/SEQ ID NO: 256; the CRISPR nuclease/gRNA pair of SEQ ID NO:71/SEQ ID NO: 211 or the CRISPR nuclease/gRNA pair of SEQ ID NO:71/SEQ ID NO: 257; the CRISPR nuclease/gRNA pair of SEQ ID NO:72/SEQ ID NO: 212 or the CRISPR nuclease/gRNA pair of SEQ ID NO:72/SEQ ID NO: 258; the CRISPR nuclease/gRNA pair of SEQ ID NO:73/SEQ ID NO: 213 or the CRISPR nuclease/gRNA pair of SEQ ID NO:73/SEQ ID NO: 259; the CRISPR nuclease/gRNA pair of SEQ ID NO:74/SEQ ID NO: 214 or the CRISPR nuclease/gRNA pair of SEQ ID NO:74/SEQ ID NO: 260; the CRISPR nuclease/gRNA pair of SEQ ID NO:75/SEQ ID NO: 215 or the CRISPR nuclease/gRNA pair of SEQ ID NO:75/SEQ ID NO: 261; the CRISPR nuclease/gRNA pair of SEQ ID NO:76/SEQ ID NO: 216 or the CRISPR nuclease/gRNA pair of SEQ ID NO:76/SEQ ID NO: 262; the CRISPR nuclease/gRNA pair of SEQ ID NO:77/SEQ ID NO: 217 or the CRISPR nuclease/gRNA pair of SEQ ID NO:77/SEQ ID NO: 263; the CRISPR nuclease/gRNA pair of SEQ ID NO:78/SEQ ID NO: 218 or the CRISPR nuclease/gRNA pair of SEQ ID NO:78/SEQ ID NO: 264; the CRISPR nuclease/gRNA pair of SEQ ID NO:79/SEQ ID NO: 219 or the CRISPR nuclease/gRNA pair of SEQ ID NO:79/SEQ ID NO: 265; the CRISPR nuclease/gRNA pair of SEQ ID NO:80/SEQ ID NO: 220 or the CRISPR nuclease/gRNA pair of SEQ ID NO:80/SEQ ID NO: 266; the CRISPR nuclease/gRNA pair of SEQ ID NO:81/SEQ ID NO: 221 or the CRISPR nuclease/gRNA pair of SEQ ID NO:81/SEQ ID NO: 267; the CRISPR nuclease/gRNA pair of SEQ ID NO:82/SEQ ID NO: 222 or the CRISPR nuclease/gRNA pair of SEQ ID NO:82/SEQ ID NO: 268; the CRISPR nuclease/gRNA pair of SEQ ID NO:83/SEQ ID NO: 223 or the CRISPR nuclease/gRNA pair of SEQ ID NO:83/SEQ ID NO: 269; the CRISPR nuclease/gRNA pair of SEQ ID NO:84/SEQ ID NO: 224 or the CRISPR nuclease/gRNA pair of SEQ ID NO:84/SEQ ID NO: 270; the CRISPR nuclease/gRNA pair of SEQ ID NO:85/SEQ ID NO: 225 or the CRISPR nuclease/gRNA pair of SEQ ID NO:85/SEQ ID NO: 271; the CRISPR nuclease/gRNA pair of SEQ ID NO:86/SEQ ID NO: 226 or the CRISPR nuclease/gRNA pair of SEQ ID NO:86/SEQ ID NO: 272; the CRISPR nuclease/gRNA pair of SEQ ID NO:87/SEQ ID NO: 227 or the CRISPR nuclease/gRNA pair of SEQ ID NO:87/SEQ ID NO: 273; the CRISPR nuclease/gRNA pair of SEQ ID NO:88/SEQ ID NO: 228 or the CRISPR nuclease/gRNA pair of SEQ ID NO:88/SEQ ID NO: 274; the CRISPR nuclease/gRNA pair of SEQ ID NO:89/SEQ ID NO: 229 or the CRISPR nuclease/gRNA pair of SEQ ID NO:89/SEQ ID NO: 275; the CRISPR nuclease/gRNA pair of SEQ ID NO:90/SEQ ID NO: 230 or the CRISPR nuclease/gRNA pair of SEQ ID NO:90/SEQ ID NO: 276; the CRISPR nuclease/gRNA pair of SEQ ID NO:91/SEQ ID NO: 231 or the CRISPR nuclease/gRNA pair of SEQ ID NO:91/SEQ ID NO: 277; the CRISPR nuclease/gRNA pair of SEQ ID NO:92/SEQ ID NO: 232 or the CRISPR nuclease/gRNA pair of SEQ ID NO:92/SEQ ID NO: 278; and/or the CRISPR nuclease/gRNA pair of SEQ ID NO:93/SEQ ID NO: 233 or the CRISPR nuclease/gRNA pair of SEQ ID NO:93/SEQ ID NO: 279. In specific instances, the CRISPR nuclease/gRNA pair is SEQ ID NO:22/SEQ ID NO: 115 or the CRISPR nuclease/gRNA pair of SEQ ID NO:22/SEQ ID NO: 138; the CRISPR nuclease/gRNA pair of SEQ ID NO:62/SEQ ID NO: 202 or the CRISPR nuclease/gRNA pair of SEQ ID NO:62/SEQ ID NO: 248; the CRISPR nuclease/gRNA pair of SEQ ID NO:80/SEQ ID NO: 220 or the CRISPR nuclease/gRNA pair of SEQ ID NO:80/SEQ ID NO: 266; and/or the CRISPR nuclease/gRNA pair of SEQ ID NO:91/SEQ ID NO: 231 or the CRISPR nuclease/gRNA pair of SEQ ID NO:91/SEQ ID NO: 277. In preferred cases, the nucleic acid-guided CRISPR nuclease has trans-cleavage activity. In certain, cases the nucleic acid-guided CRISPR nuclease can hybridize to a PAM region in a target nucleic acid sequence. In some instances, the substrate for the nucleic acid-guided CRISPR nuclease is a ribonucleic acid molecule (RNA) complexed with a fluorophore and a quencher for the fluorophore. In other cases the substrate is a ribonucleic acid molecule (RNA) complexed with biotin. In yet other instances, the substrate can be a deoxyribonucleic acid (DNA) complexed with a detection moiety. The substrate can be present in the reaction mixtures in a range of concentrations ranging, e.g., from 250 nM to 450 nM.

In some instances, the reaction mixture(s) and/or enzyme systems described herein comprise pairs, each comprising a complex of a certain CRISPR nuclease and a gRNA, wherein the gRNA comprises a sequence (e.g., a gRNA sequence), and wherein the gRNA sequence comprises a direct repeat sequence. In some CRISPR nuclease/gRNA complex embodiments, the homology of the CRISPR nuclease (e.g., enzyme) may be at least 85%, at least 90%, at least 95%, or at least 99% amino acid homology to any one of SEQ ID NOs:1-93. In some CRISPR nuclease/gRNA complex embodiments, the gRNA sequence may comprise a direct repeat sequence having at least 85%, at least 90%, at least 95%, or at least 99% amino acid homology to any one of SEQ ID NOs:374-466. In some CRISPR nuclease/gRNA complex embodiments, each of the CRISPR nuclease and the direct repeat sequence of the gRNA may independently have at least 85%, at least 90%, at least 95%, or at least 99% homology to the sequences of a CRISPR nuclease/direct repeat pair selected from the group consisting of: the CRISPR nuclease/direct repeat pair of SEQ ID NO:1/SEQ ID NO:374; the CRISPR nuclease/direct repeat pair of SEQ ID NO:2/SEQ ID NO: 375; the CRISPR nuclease/direct repeat pair of SEQ ID NO:3/SEQ ID NO: 376; the CRISPR nuclease/direct repeat pair of SEQ ID NO:4/SEQ ID NO:377; the CRISPR nuclease/direct repeat pair of SEQ ID NO:5/SEQ ID NO:378; the CRISPR nuclease/direct repeat pair of SEQ ID NO:6/SEQ ID NO:379; the CRISPR nuclease/direct repeat pair of SEQ ID NO:7/SEQ ID NO:380; the CRISPR nuclease/direct repeat pair of SEQ ID NO:8/SEQ ID NO:381; the CRISPR nuclease/direct repeat pair of SEQ ID NO:9/SEQ ID NO:382; the CRISPR nuclease/direct repeat pair of SEQ ID NO: 10/SEQ ID NO:383; the CRISPR nuclease/direct repeat pair of SEQ ID NO: 11/SEQ ID NO:384; the CRISPR nuclease/direct repeat pair of SEQ ID NO:12/SEQ ID NO:385; the CRISPR nuclease/direct repeat pair of SEQ ID NO:13/SEQ ID NO:386; the CRISPR nuclease/direct repeat pair of SEQ ID NO:14/SEQ ID NO:387; the CRISPR nuclease/direct repeat pair of SEQ ID NO: 15/SEQ ID NO:388; the CRISPR nuclease/direct repeat pair of SEQ ID NO:16/SEQ ID NO:389; the CRISPR nuclease/direct repeat pair of SEQ ID NO:17/SEQ ID NO:390; the CRISPR nuclease/direct repeat pair of SEQ ID NO:18/SEQ ID NO:391; the CRISPR nuclease/direct repeat pair of SEQ ID NO: 19/SEQ ID NO:392; the CRISPR nuclease/direct repeat pair of SEQ ID NO:20/SEQ ID NO:393; the CRISPR nuclease/direct repeat pair of SEQ ID NO:21/SEQ ID NO:394; the CRISPR nuclease/direct repeat pair of SEQ ID NO:22/SEQ ID NO:395; the CRISPR nuclease/direct repeat pair of SEQ ID NO:23/SEQ ID NO:396; the CRISPR nuclease/direct repeat pair of SEQ ID NO:24/SEQ ID NO:397; the CRISPR nuclease/direct repeat pair of SEQ ID NO:25/SEQ ID NO:398; the CRISPR nuclease/direct repeat pair of SEQ ID NO:26/SEQ ID NO:399; the CRISPR nuclease/direct repeat pair of SEQ ID NO:27/SEQ ID NO:400; the CRISPR nuclease/direct repeat pair of SEQ ID NO:28/SEQ ID NO:401; the CRISPR nuclease/direct repeat pair of SEQ ID NO:29/SEQ ID NO:402; the CRISPR nuclease/direct repeat pair of SEQ ID NO:30/SEQ ID NO:403; the CRISPR nuclease/direct repeat pair of SEQ ID NO:31/SEQ ID NO:404; the CRISPR nuclease/direct repeat pair of SEQ ID NO:32/SEQ ID NO:405; the CRISPR nuclease/direct repeat pair of SEQ ID NO:33/SEQ ID NO:406; the CRISPR nuclease/direct repeat pair of SEQ ID NO:34/SEQ ID NO:407; the CRISPR nuclease/direct repeat pair of SEQ ID NO:35/SEQ ID NO:408; the CRISPR nuclease/direct repeat pair of SEQ ID NO:36/SEQ ID NO:409; the CRISPR nuclease/direct repeat pair of SEQ ID NO:37/SEQ ID NO:410; the CRISPR nuclease/direct repeat pair of SEQ ID NO:38/SEQ ID NO:411; the CRISPR nuclease/direct repeat pair of SEQ ID NO:39/SEQ ID NO:412; the CRISPR nuclease/direct repeat pair of SEQ ID NO:40/SEQ ID NO:413; the CRISPR nuclease/direct repeat pair of SEQ ID NO:41/SEQ ID NO:414; the CRISPR nuclease/direct repeat pair of SEQ ID NO:42/SEQ ID NO:415; the CRISPR nuclease/direct repeat pair of SEQ ID NO:43/SEQ ID NO:416; the CRISPR nuclease/direct repeat pair of SEQ ID NO:44/SEQ ID NO:417; the CRISPR nuclease/direct repeat pair of SEQ ID NO:45/SEQ ID NO:418; the CRISPR nuclease/direct repeat pair of SEQ ID NO:46/SEQ ID NO:419; the CRISPR nuclease/direct repeat pair of SEQ ID NO:47/SEQ ID NO:420; the CRISPR nuclease/direct repeat pair of SEQ ID NO:48/SEQ ID NO:421; the CRISPR nuclease/direct repeat pair of SEQ ID NO:49/SEQ ID NO:422; the CRISPR nuclease/direct repeat pair of SEQ ID NO:50/SEQ ID NO:423; the CRISPR nuclease/direct repeat pair of SEQ ID NO:51/SEQ ID NO:424; the CRISPR nuclease/direct repeat pair of SEQ ID NO:52/SEQ ID NO:425; the CRISPR nuclease/direct repeat pair of SEQ ID NO:53/SEQ ID NO:426; the CRISPR nuclease/direct repeat pair of SEQ ID NO:54/SEQ ID NO:427; the CRISPR nuclease/direct repeat pair of SEQ ID NO:55/SEQ ID NO:428; the CRISPR nuclease/direct repeat pair of SEQ ID NO:56/SEQ ID NO:429; the CRISPR nuclease/direct repeat pair of SEQ ID NO:57/SEQ ID NO:430; the CRISPR nuclease/direct repeat pair of SEQ ID NO:58/SEQ ID NO:431; the CRISPR nuclease/direct repeat pair of SEQ ID NO:59/SEQ ID NO:432; the CRISPR nuclease/direct repeat pair of SEQ ID NO:60/SEQ ID NO:433; the CRISPR nuclease/direct repeat pair of SEQ ID NO:61/SEQ ID NO:434; the CRISPR nuclease/direct repeat pair of SEQ ID NO:62/SEQ ID NO:435; the CRISPR nuclease/direct repeat pair of SEQ ID NO:63/SEQ ID NO:436; the CRISPR nuclease/direct repeat pair of SEQ ID NO:64/SEQ ID NO:437; the CRISPR nuclease/direct repeat pair of SEQ ID NO:65/SEQ ID NO:438; the CRISPR nuclease/direct repeat pair of SEQ ID NO:66/SEQ ID NO:439; the CRISPR nuclease/direct repeat pair of SEQ ID NO:67/SEQ ID NO:440; the CRISPR nuclease/direct repeat pair of SEQ ID NO:68/SEQ ID NO:441; the CRISPR nuclease/direct repeat pair of SEQ ID NO:69/SEQ ID NO:442; the CRISPR nuclease/direct repeat pair of SEQ ID NO:70/SEQ ID NO:443; the CRISPR nuclease/direct repeat pair of SEQ ID NO:71/SEQ ID NO:444; the CRISPR nuclease/direct repeat pair of SEQ ID NO:72/SEQ ID NO:445; the CRISPR nuclease/direct repeat pair of SEQ ID NO:73/SEQ ID NO:446; the CRISPR nuclease/direct repeat pair of SEQ ID NO:74/SEQ ID NO:447; the CRISPR nuclease/direct repeat pair of SEQ ID NO:75/SEQ ID NO:448; the CRISPR nuclease/direct repeat pair of SEQ ID NO:76/SEQ ID NO:449; the CRISPR nuclease/direct repeat pair of SEQ ID NO:77/SEQ ID NO:450; the CRISPR nuclease/direct repeat pair of SEQ ID NO:78/SEQ ID NO:451; the CRISPR nuclease/direct repeat pair of SEQ ID NO:79/SEQ ID NO:452; the CRISPR nuclease/direct repeat pair of SEQ ID NO:80/SEQ ID NO:453; the CRISPR nuclease/direct repeat pair of SEQ ID NO:81/SEQ ID NO:454; the CRISPR nuclease/direct repeat pair of SEQ ID NO:82/SEQ ID NO:455; the CRISPR nuclease/direct repeat pair of SEQ ID NO:83/SEQ ID NO:456; the CRISPR nuclease/direct repeat pair of SEQ ID NO:84/SEQ ID NO:457; the CRISPR nuclease/direct repeat pair of SEQ ID NO:85/SEQ ID NO:458; the CRISPR nuclease/direct repeat pair of SEQ ID NO:86/SEQ ID NO:459; the CRISPR nuclease/direct repeat pair of SEQ ID NO:87/SEQ ID NO:460; the CRISPR nuclease/direct repeat pair of SEQ ID NO:88/SEQ ID NO:461; the CRISPR nuclease/direct repeat pair of SEQ ID NO:89/SEQ ID NO:462; the CRISPR nuclease/direct repeat pair of SEQ ID NO:90/SEQ ID NO:463; the CRISPR nuclease/direct repeat pair of SEQ ID NO:91/SEQ ID NO:464; the CRISPR nuclease/direct repeat pair of SEQ ID NO:92/SEQ ID NO:465; or the CRISPR nuclease/direct repeat pair of SEQ ID NO:93/SEQ ID NO:466. Any of the reaction mixtures, expression vectors, enzyme systems, methods may comprise, encode, and/or use any one or more of the CRISPR nuclease/gRNA complexes described herein, including in the foregoing.

In some instances the reaction mixture(s) or enzymatic systems may further comprises a target nucleic acid sequence, e.g., a sequence from an influenza A virus (e.g., SEQ ID NO:373), an influenza B virus, a Respiratory Syncytial Virus (RSV), or a SARS-CoV-2 virus. In some instances, the target nucleic acid sequence activates cleavage of the substrate by the nucleic acid-guided CRISPR nuclease. Cleavage can occur, e.g., with a sensitivity of at least 25 nM. A nucleic acid-guided CRISPR nuclease can be present in the reaction mix in a concentration ranging from 10 ng/uL to 100 ng/uL. A guide nucleic acid (gRNA) can be present in the reaction mix in a concentration ranging from 20 nM to 180 nM. Suitable gRNAs can be from 40 nucleotides to 60 nucleotides in length. The reaction mixture(s) described herein may further comprise an RNase inhibitor and MgCl₂. The reaction mixture(s) described herein may also comprise a crRNA.

In some instances the disclosure provides reagents for the manufacture of components of reaction mixture(s) and enzymatic mix(es) described herein. In some instances, one or more expression vector(s) encode a nucleic acid-guided CRISPR nuclease having at least 85% amino acid homology to any one of SEQ ID NOs:1-93 from a heterologous promoter. In some instances, one or more expression vector(s) encode a direct repeat that has the same, or substantially the same sequence to any one of SEQ ID NOs:374-466 from a heterologous promoter. The heterologous promoter can be, e.g., a T7 promoter. In certain configurations the expression vector further comprises a purification tag, e.g., a 6-his tag. In the cases where a purification tag is present, the purification tag can be present on an N-terminus of the nucleic acid-guided CRISPR nuclease, on a C-terminus of the nucleic acid-guided CRISPR nuclease, or in both termini. The purification tag can be cleavable. A recombinant nucleic acid-guided CRISPR nuclease can be produced, e.g., from a cell-free expression system from an expression vector. Alternatively, the recombinant nucleic acid-guided CRISPR nuclease can be produced in a bacterial expression system. In certain cases, the recombinant nucleic acid-guided CRISPR nuclease is combined with gRNA comprising a direct repeat sequence selected from group consisting of one or more of SEQ ID NO:374-466. In certain cases, the recombinant nucleic acid-guided CRISPR nuclease has trans-cleavage activity, it may cleave a single stranded deoxyribonucleic acid (DNA) molecule, a double stranded deoxyribonucleic acid (DNA) molecule, or a ribonucleic acid (RNA) molecule. In one particular instance, a recombinant nucleic acid-guided CRISPR nuclease, produced in a cell-free expression system, can exhibit at least 25% higher cleavage activity as compared to LwaCas13a when cleaving SEQ ID NO: 180. Recombinant nucleic acid-guided CRISPR nucleases described herein have predicted molecular weights of 90 kDa to 170 kDa when produced from cell-free expression systems.

In some aspects, the disclosure provides one or more nucleic acid sequence(s) having at least 80%, at least 85%, at least 90%, or at least 95% sequence identity to any one of SEQ ID NOs:280-372, wherein the nucleic acid sequence encodes a nucleic acid-guided CRISPR nuclease comprising an amino acid sequence of any one of SEQ ID NOs:1-93 and a heterologous sequence, e.g., a heterologous promoter sequence or a heterologous sequence encoding a purification tag. The nucleic acid sequence can be recombinantly expressed in an organism or it can be produced in a cell-free expression system. In certain embodiments, the nucleic acid sequence is codon optimized for expression in E. coli.

In some aspects the disclosure provides a method of detecting a target nucleic acid, the method comprising: contacting a sample with a nucleic acid-guided nuclease system comprising: a nucleic acid-guided CRISPR nuclease having at least 90% identity to SEQ ID NO: 1-93, or a nucleic acid molecule encoding the nucleic acid-guided CRISPR nuclease; a guide nucleic acid for complexing with the nucleic acid-guided CRISPR nuclease, or a nucleic acid molecule encoding the guide nucleic acid; and allowing the target nucleic acid to activate the nucleic acid-guided CRISPR nuclease system to cleave a detectable substrate. In some aspects, the guide nucleic acid comprises SEQ ID NO. 115 or SEQ ID No. 138 when designed for targeting FluA. The disclosure contemplates other guide nucleic acid sequences comprising a direct repeat selected from any one of SEQ ID NO:374 to SEQ ID NO:466 and a spacer sequence designed for hybridizing with a pre-selected targeting region.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 (FIG. 1A) illustrates a rapid and high throughput workflow for the discovery of novel CRISPR-Cas proteins.

FIG. 1 (FIG. 1B) illustrates representative steps of the method employed to identify novel enzymes from sequences that do not share significant homology to existing enzymes.

FIG. 2 (FIG. 2) is a chart illustrating the results of an activity screening of initial 23 candidates with NTC (negative control). Activity was measured by the fluorescence released upon digestion of RNase Alert RNA substrate in the system.

FIG. 3 (FIG. 3) is a chart illustrating the results of activity confirmation of candidate JW13124 (SEQ ID NO: 22). JW13124 demonstrated a superior activity profile compared to the wild-type LwaCas13a under current testing conditions. Activity was measured by the fluorescence released upon digestion of an RNase Alert RNA substrate in the system.

FIG. 4 (FIG. 4) is a chart illustrating that collateral (i.e., trans-cleavage) activity of JW13124 (SEQ ID NO: 22) was only activated in the presence of both its corresponding guide RNA and the target sequence. Activity was measure by the fluorescence released upon digestion of RNase Alert RNA substrate in the system.

FIG. 5 (FIG. 5) is a chart illustrating the results of activity confirmation of candidate JW13166 (SEQ ID NO:62). JW13166 demonstrated a superior activity profile compared to the wild-type LwaCas13a under current testing conditions. Activity was measured by the fluorescence released upon digestion of an RNase Alert RNA substrate in the system.

FIG. 6 (FIG. 6) is a chart illustrating that collateral (i.e., trans-cleavage) activity of JW13166 (SEQ ID NO:62) was only activated in the presence of both its corresponding guide RNA and the target sequence. Activity was measure by the fluorescence released upon digestion of RNase Alert RNA substrate in the system.

FIG. 7 (FIG. 7) is a chart illustrating the results of activity confirmation of candidate JW13184 (SEQ ID NO:80). Activity was measured by the fluorescence released upon digestion of an RNase Alert RNA substrate in the system.

FIG. 8 (FIG. 8) is a chart illustrating collateral (i.e., trans-cleavage) activity of JW13184 (SEQ ID NO:80) in the presence of both its corresponding guide RNA and the target sequence. Activity was measure by the fluorescence released upon digestion of RNase Alert RNA substrate in the system.

FIG. 9 (FIG. 9) is a chart illustrating the results of activity confirmation of candidate JW13195 (SEQ ID NO:91). JW13195 demonstrated a superior activity profile compared to the wild-type LwaCas13a under current testing conditions. Activity was measured by the fluorescence released upon digestion of an RNase Alert RNA substrate in the system.

FIG. 10 (FIG. 10) is a chart illustrating that collateral (i.e., trans-cleavage) activity of JW13195 (SEQ ID NO:91) was only activated in the presence of both its corresponding guide RNA and the target sequence. Activity was measure by the fluorescence released upon digestion of RNase Alert RNA substrate in the system.

FIG. 11a (FIG. 11a) illustrate images of lateral flow strips used to detect RNA cleavage activity of nucleic acid-guided CRISPR nucleases and demonstrating that JW13166 (SEQ ID NO:62) produces measurable target RNA cleavage activity after an incubation time of 2 minutes at both 25° C. and 37° C. whereas wild-type LwaCas13a does not produce measurable target RNA cleavage activity after 2 minutes at either incubation time.

FIG. 11b (FIG. 11b) illustrate images of lateral flow strips used to detect RNA cleavage activity of nucleic acid-guided CRISPR nucleases and demonstrating that JW13166 (SEQ ID NO:62) produces measurable target RNA cleavage activity after an incubation time of 60 minutes at both 25° C. and 37° C. whereas wild-type LwaCas13a only produces measurable target RNA cleavage activity at an incubation temperature of 37° C.

It should be understood that the drawings are not necessarily to scale, and that like reference numbers refer to like features.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Terminology

As used herein the abbreviation “CRISPR” generally refers to Clustered Regularly Interspaced Short Palindromic Repeats. Although there are diverse CRISPR-Cas systems among the different species of archaea and bacteria, these systems are generally connected by their dependence on a single-stranded RNA molecule (crRNA), which guides CRISPR proteins to recognize and cleave nucleic acid targets. The crRNA can be programmed towards a specific DNA or RNA region of interest through hybridization to a complementary sequence, which in some systems is restricted to the proximity of a protospacer adjacent motif (PAM) or protospacer flanking sequence. The term “CRISPR nuclease” refers to members of the CRISPR-Cas system. As used herein the term “CRISPR complex” or “complex” refers to a CRISPR nuclease complexed with a guide nucleic acid (gRNA).

As used herein, the term, “gRNA” refers to a ribonucleic acid sequence with a conserved sequence forming one or more stem-loop structure(s) (e.g., a direct repeat or “DR”) and a spacer sequence. Generally, the CRISPR nuclease binds to the DR in the “CRISPR complex” or “complex”, and the spacer sequence comprises a sequence that is complementary to the target nucleic acid.

As used herein the term, “CRISPR nuclease/gRNA pair”, refers to a complex comprising at a CRISPR nuclease and gRNA designed for complexing with the nuclease.

As used herein the term, “the CRISPR nuclease/direct repeat pair”, refers to a complex comprising at a CRISPR nuclease and gRNA comprising at least one direct repeat sequence for complexing with the CRISPR nuclease. It is to be understood that the gRNA may comprise other regions for its functionality, including a “spacer” region.

As used herein the abbreviation “RSV” generally refers to Respiratory Syncytial Virus (RSV).

As used herein the abbreviations “FluA” and “FluB” generally refer to Influenza A and Influenza B, the two main types of influenza viruses that cause seasonal flu outbreaks. As used herein the term “Flu” refers to all viruses in the Influenza family.

As used herein the abbreviations “SARS-CoV-2” refers to the virus causing COVID-19.

As used herein, certain terms may have the following defined meanings. As used in the specification and claims, the singular form “a,” “an” and “the” include singular and plural references unless the context clearly dictates otherwise. For example, the term “a nucleic acid” includes a single nucleic acid molecule as well as a plurality of nucleic acids, including mixtures thereof.

As used herein, “about” means the recited quantity exactly and small variations within a limited range encompassing plus or minus 10% of the recited quantity. In other words, the limited range encompassed can include ±10%, 9%, 8%, 7%, ±6%, 5, 4%, 3%, 2%, 1%, 0.5%, 0.2%, ±0.1%, +0.05%, or smaller, as well as the recited value itself. Thus, by way of example, “about 10” should be understood to mean “10” and a range no larger than “9-11”.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the term “percent sequence identity” with respect to a reference nucleic acid sequence is the percentage of nucleic acid bases in a target sequence that are identical with the nucleic acid bases in the reference sequence, respectively, after aligning the sequences and introducing gaps, if necessary, to achieve the maximum percent sequence identity. Methods of sequence alignment are well known in the art. Optimal alignment of sequences can be conducted by methods described in Needleman and Wunsch, 1970, J. Mol. Biol. 48:443; Pearson and Lipman, 1988, PNAS 85:2444, by computerized implementations of these algorithms. Alignments can be made using publicly available computer software such as BLASTp, BLASTn, BLAST-2, ALIGN or MegAlign Pro (DNASTAR) software.

As used herein the term “sample”, generally refers to any source of nucleic acids—either from an specimen, from a subject “hosting” the specimen or both—that can be informative of an environment. It may refer to samples derived from a subject, such as a nasal swab, blood, plasma, urine, tissue, faces, bone marrow, saliva, cerebrospinal fluid, or any other suitable tissue sample. It may refer to swab samples that are collected from surfaces in food processing facilities, long-term care facilities, hospitals, restaurants, or any suitable surface comprising nucleic acids. It may refer to a sample that comprises a biological tissue, soil, water, air, air filter materials, animal production, feed, manure, crop production, manufacturing plants, or any other suitable samples. Such samples may be derived from a hospital or a clinic and they may be analyzed on a mobile platform.

As used herein, the term “subject,” can refer to a human or to another animal. An animal can be a mouse, a rat, a guinea pig, a dog, a cat, a horse, a rabbit, and various other animals. A subject can be of any age, for example, a subject can be an infant, a toddler, a child, a pre-adolescent, an adolescent, an adult, or an elderly individual.

### DETAILED DESCRIPTIONS

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

Described herein are reaction mixtures, enzyme systems, fluidic devices, kits, and methods for detection of target nucleic acids associated with various diseases. The reaction mixtures, enzyme systems, fluidic devices, kits, and methods described herein may comprise reagents of a guide nucleic acid targeting a target nucleic acid, a nucleic acid-guided CRISPR nuclease (i.e., a CRISPR nuclease), and a single stranded detector nucleic acid with a detection moiety. The target nucleic acid may be cleaved by a complex that is defined by a nucleic acid-guided CRISPR nuclease (CRISPR nuclease) complexed with a guide nucleic acid (gRNA), i.e., a nucleic acid-guided CRISPR nuclease/gRNA pair. The gRNA in the complex typically comprises a direct repeat sequence for interacting with the CRISPR enzyme, and a spacer sequence. The disclosure contemplates various CRISPR complexes, including, e.g., complexes targeting FluA, FluB, RSV, and Sars-CoV-2. Illustrative examples are provided throughout targeting FluA.

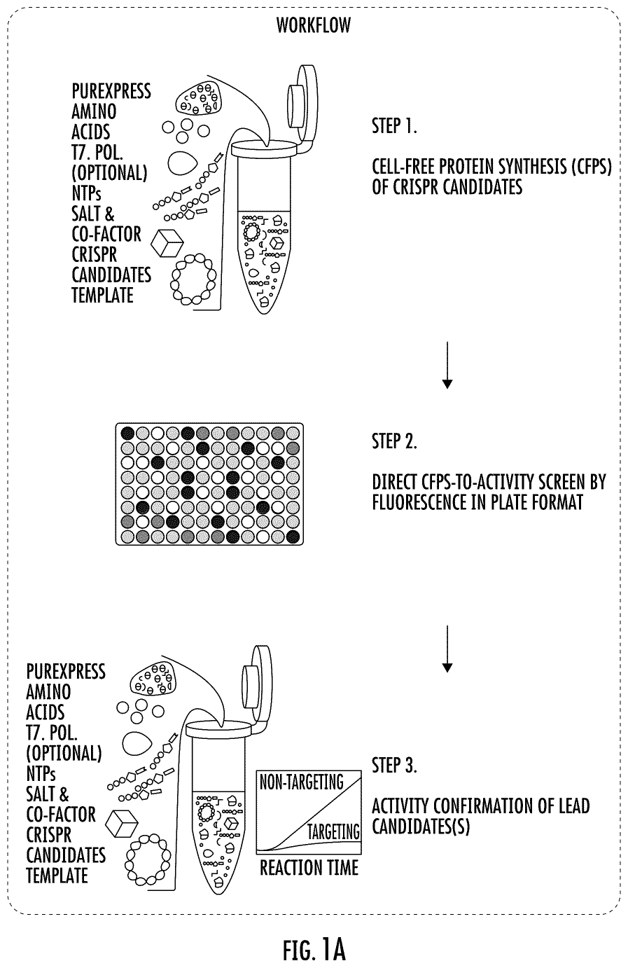

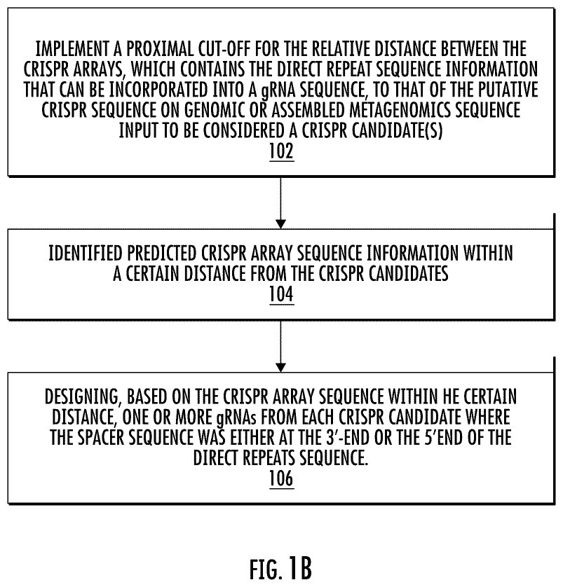

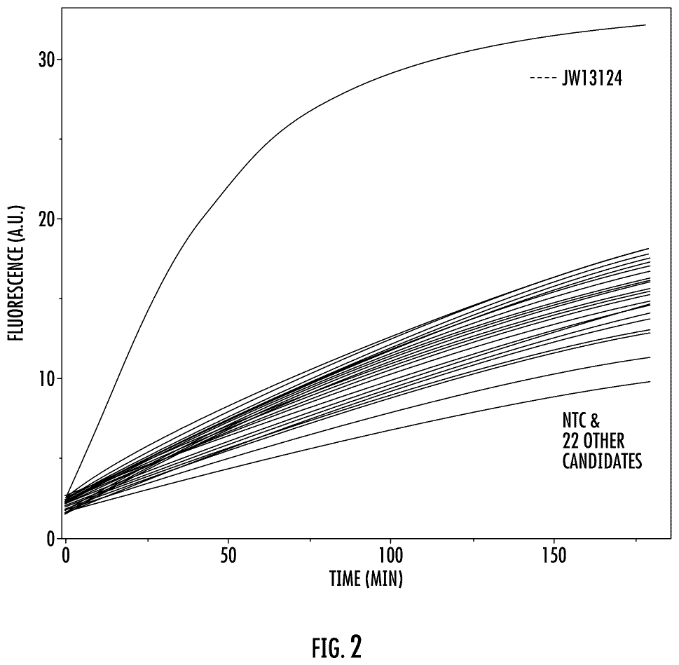

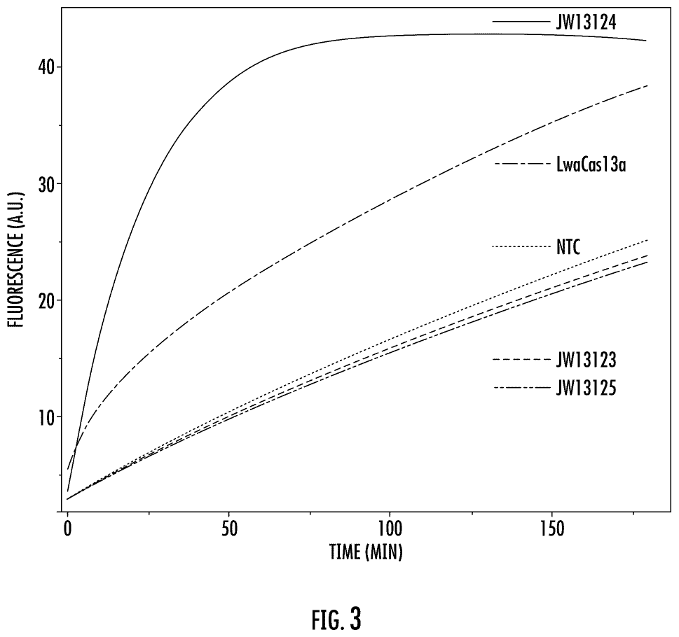

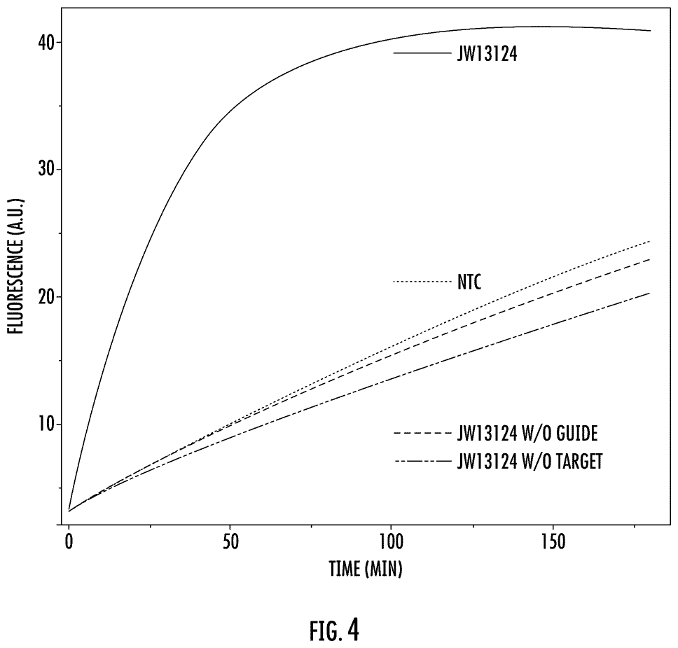

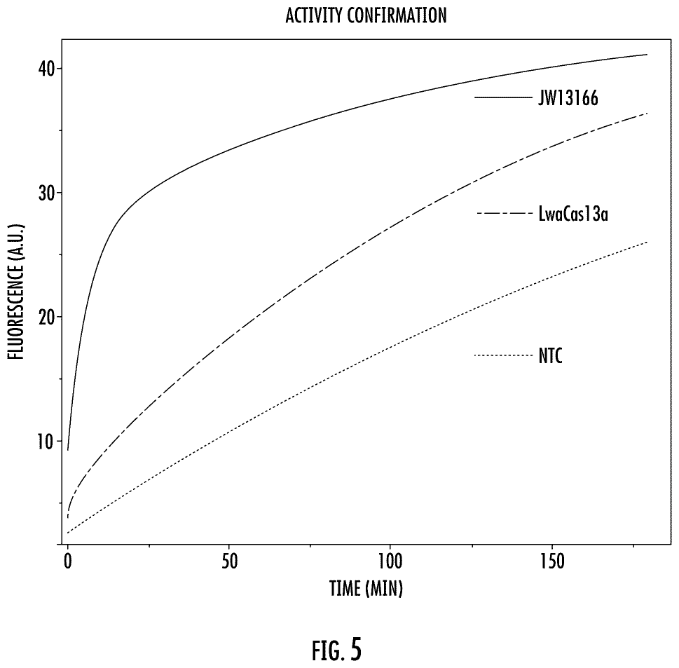

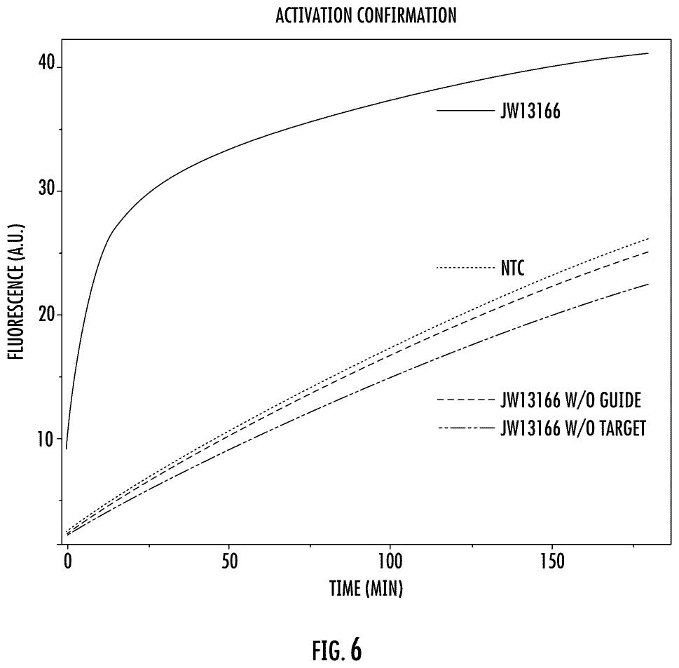

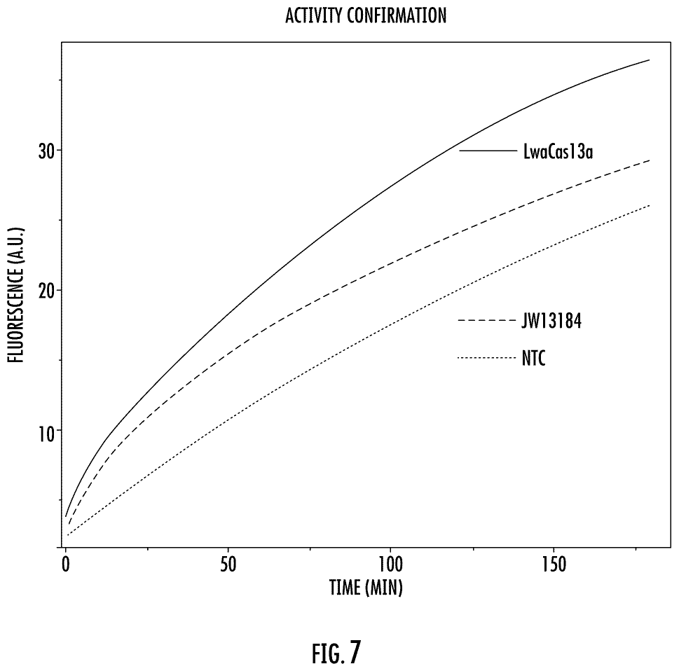

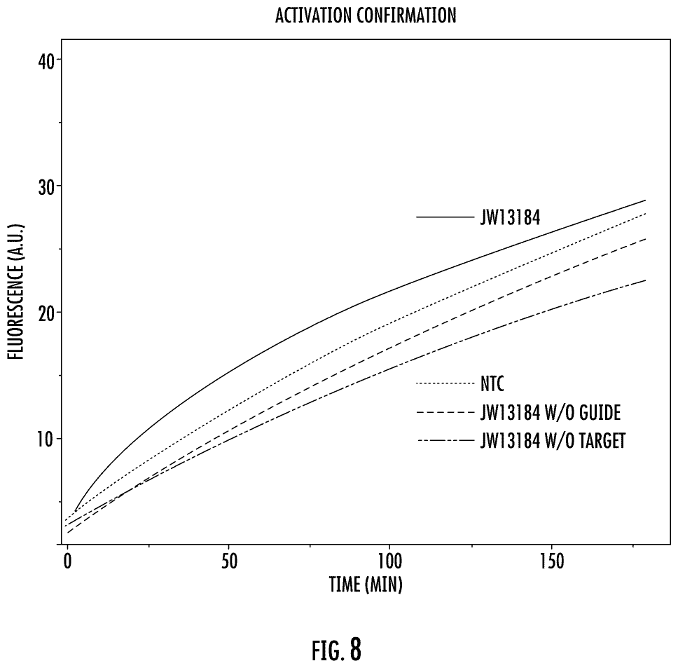

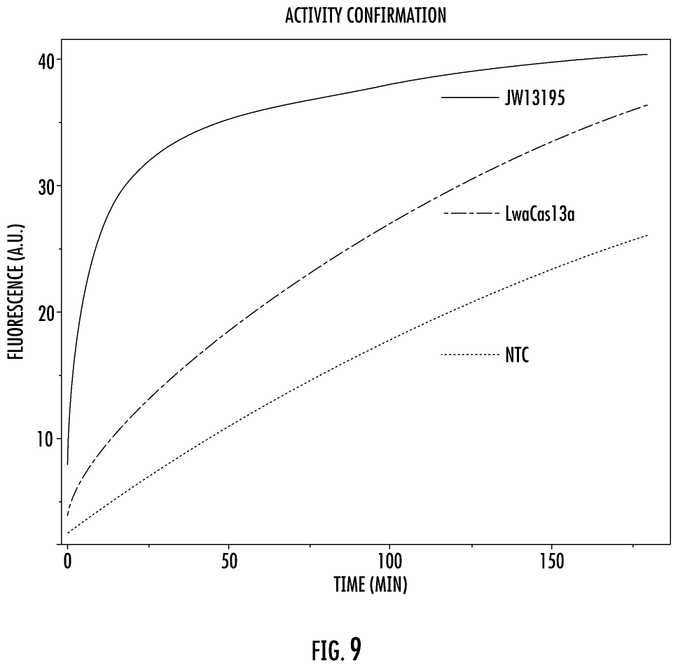

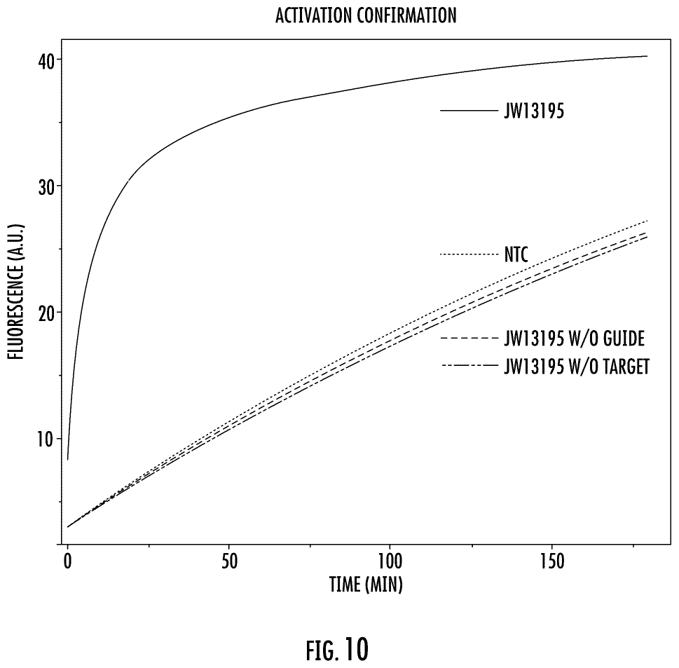

The disclosure describes the development of a rapid and high throughput workflow for the discovery of novel CRISPR-Cas proteins (FIG. 1). Preliminary candidates were mined and evaluated from public database (e.g. GenBank, SGB, MGnify, etc.), and a representative subsets of the candidates (93 candidates) were designed into Cell-Free Protein Synthesis (CFPS) compatible expression templates for downstream protein expression. A fluorescence-based high-throughput activity assay was then used to differentiate potential active candidate(s) from negative control samples. During initial screening rounds with a preliminary number of candidate CRISPR nuclease/gRNA complexes, four candidates, were identified to be active in the conditions tested when complexed with the tested gRNA (FIG. 2, FIG. 3, FIG. 5, FIG. 7, and FIG. 9). Surprisingly, when compared to a wild-type positive control CRISPR-Cas protein, LwaCas13a, candidates JW13124 (SEQ ID NO: 22), JW13166 (SEQ ID NO: 62), and JW13195 (SEQ ID NO: 91) were able to demonstrate a superior activity profile (FIGS. 3, FIG. 5, FIG. 7, and FIG. 9). Additionally, candidates JW13124 (SEQ ID NO: 22), JW13166 (SEQ ID NO: 62), and JW13195 (SEQ ID NO: 91) were only activated in the presence of both its corresponding guide RNA and its intended target sequence (FIG. 4, FIG. 6, and FIG. 10). Candidate JW13184 (SEQ ID NO: 80) was also found to display activity, although a distinct activity profile (FIG. 8).

### CRISPR Enzymes

Since their initial discovery, the number of different CRISPR-Cas systems has expanded rapidly. Currently, CRISPR-Cas systems can be divided, according to evolutionary relationships, into two classes, six types and several subtype. The classes of CRISPR-Cas system are defined by the nature of the ribonucleoprotein effector complex: class 1 systems are characterized by a complex of multiple effector proteins, and class 2 systems encompass a single crRNA-binding protein. The main difference between CRISPR type II (Cas9) systems and type V (Cas12) and type VI (Cas13) systems is the ability of the latter two systems to trigger non-specific collateral cleavage (trans cleavage or trans-cleavage) upon target hybridization. Collateral activity involves the cleavage of non-targeted single-stranded DNA (ssDNA; Cas12) or single-stranded RNA (ssRNA; Cas13) in solution, which enables the sensing of nucleic acids through signal amplification and allows for various readouts through the addition of functionalized reporter nucleic acids, which are generally cleaved by collateral (trans cleavage) activity. The present disclosure relates to the discovery of novel CRISPR enzymes having a very low degree of amino acid sequence homology to any previously known CRISPR-Cas proteins (<42% amino acid sequence homology to any characterized CRISPR-Cas proteins with >65% coverage) and characterization of their function with putatively tested guide RNAs. In the discovery of the identified novel CRISPR enzymes the present disclosure employed a method that: 1) extracted putative CRISPR-Cas protein signatures (motifs and functional domains) from known enzymes; 2) surveyed public genomic and metagenomics databases with possible sequences that matches to the signature at various degrees; 3) provided de-redundancy of initial candidate pool by removing repeats and previously characterized CRISPR-Cas proteins; 4) ranked candidates by phylogenetic representations; and 5) performed activity assays of representative candidates with rapid and high-throughput cell-free protein synthesis technologies, thereby confirming activity of certain candidates. Further preliminary characterization of lead candidates was also performed to confirm activity.

Disclosed herein are nucleic acid-guided nucleases, in particular CRISPR nucleases with SEQ ID Nos:1-93. In some aspects, the nucleases described herein were discovered from an evaluation of sequences that displayed <42% amino acid sequence homology to representative (e.g., Cas9, Cas12, Cas13), CRISPR-Cas systems in a high throughput sequence screen from databases including GenBank, SGB, and MGnify. The high throughput screen also identified 93 putative direct repeat sequences that have been tested for forming active complexes with their respective enzymes. Also disclosed herein are putative direct repeat sequences that have been discovered in the evaluation of the enzymes above. The direct repeat sequences of SEQ ID NOs:374-466 were identified as sequences that could potentially form complexes with the enzymes of SEQ ID Nos: 1-93.

Suitable nucleic acid-guided nucleases can be from an organism from a genus which includes but is not limited to Thiomicrospira, Succinivibrio, Candidatus, Porphyromonas, Acidaminococcus, Acidomonococcus, Prevotella, Smithella, Moraxella, Synergistes, Francisella, Leptospira, Catenibacterium, Kandleria, Clostridium, Dorea, Coprococcus, Enterococcus, Fructobacillus, Weissella, Pediococcus, Corynebacter, Sutterella, Legionella, Treponema, Roseburia, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Alicyclobacillus, Brevibacilus, Bacillus, Bacteroidetes, Brevibacilus, Carnobacterium, Clostridiaridium, Clostridium, Desulfonatronum, Desulfovibrio, Helcococcus, Leptotrichia, Listeria, Methanomethyophilus, Methylobacterium, Opitutaceae, Paludibacter, Rhodobacter, Sphaerochaeta, Tuberibacillus, Oleiphilus, Omnitrophica, Parcubacteria, and Campylobacter. Species of organism of such a genus can be as otherwise herein discussed. Suitable nucleic acid-guided nucleases can be from an organism from a genus or unclassified genus within a kingdom which includes but is not limited to Firmicute, Actinobacteria, Bacteroidetes, Proteobacteria, Spirochates, and Tenericutes. Suitable nucleic acid-guided nucleases can be from an organism from a genus or unclassified genus within a phylum which includes but is not limited to Erysipelotrichia, Clostridia, Bacilli, Actinobacteria, Bacteroidetes, Flavobacteria, Alphaproteobacteria, Betaproteobacteria, Gammaproteobacteria, Deltaproteobacteria, Epsilonproteobacteria, Spirochaetes, and Mollicutes. Suitable nucleic acid-guided nucleases can be from an organism from a genus or unclassified genus within an order which includes but is not limited to Clostridiales, Lactobacillales, Actinomycetales, Bacteroidales, Flavobacteriales, Rhizobiales, Rhodospirillales, Burkholderiales, Neisseriales, Legionellales, Nautiliales, Campylobacterales, Spirochaetales, Mycoplasmatales, and Thiotrichales. Suitable nucleic acid-guided nucleases can be from an organism from a genus or unclassified genus within a family which includes but is not limited to Lachnospiraceae, Enterococcaceae, Leuconostocaceae, Lactobacillaceae, Streptococcaceae, Peptostreptococcaceae, Staphylococcaceae, Eubacteriaceae, Corynebacterineae, Bacteroidaceae, Flavobacterium, Cryomoorphaceae, Rhodobiaceae, Rhodospirillaceae, Acetobacteraceae, Sutterellaceae, Neisseriaceae, Legionellaceae, Nautiliaceae, Campylobacteraceae, Spirochaetaceae, Mycoplasmataceae, Pisciririckettsiaceae, and Francisellaceae.

In some instances, a nucleic acid-guided nuclease (CRISPR nuclease) disclosed herein comprises an amino acid sequence comprising at least 50% amino acid identity to any one of SEQ ID NO: 1-93. In some instances, a CRISPR nuclease comprises an amino acid sequence comprising at least about 10%, 20%, 30%, 40%, 50%, 60%, 65%, 70%, 75%, 80%, 85%, 90%, 95%, 99%, or 100% amino acid identity to any one of SEQ ID NO: 1-93. In some cases, the nucleic acid-guided nuclease is encoded by a sequence that comprises at least about 50%, 60%, 65%, 70%, 75%, 80%, 85%, 90%, 95%, 99% or greater nucleic acid sequence identity to any one of SEQ ID NO: 280-372. In some cases, the nucleic acid-guided nuclease comprises at least about 50%, 60%, 65%, 70%, 75%, 80%, 85%, 90%, 95%, greater than 95%, amino acid identity to SEQ ID NO: 22.

In some instances, a nucleic acid-guided nuclease disclosed herein is encoded on a nucleic acid sequence. Such a nucleic acid can be expressed in a cell-free expression system, e.g., FIG. 1. Alternatively, such a nucleic acid can be codon optimized for expression in a desired host cell. Suitable host cells can include, as non-limiting examples, prokaryotic cells such as E. coli, P. aeruginosa, B. subtilus, and V. natriegens, and eukaryotic cells such as S. cerevisiae, plant cells, insect cells, nematode cells, amphibian cells, fish cells, or mammalian cells, including human cells.

A nucleic acid sequence encoding a nucleic acid-guided nuclease can be operably linked to a promoter. Such nucleic acid sequences can be linear or circular. The nucleic acid sequences can be comprised on a larger linear or circular nucleic acid sequences that comprises additional elements such as an origin of replication, selectable or screenable marker, terminator, other components of a targetable nuclease system, such as a guide nucleic acid, or an editing or recorder cassette as disclosed herein. These larger nucleic acid sequences can be recombinant expression vectors.

### Guide Nucleic Acid (2RNA) and Complex(es)

A guide nucleic acid (gRNA) is typically a ribonucleic acid sequence with a conserved sequence forming one or more stem-loop structure(s) (e.g., a direct repeat or “DR”) and a spacer sequence. Non-limiting examples of direct repeats are provided in SEQ ID NOs: 374-466. Generally, the CRISPR nuclease binds to the DR in the “CRISPR complex” or “complex”, and the spacer sequence comprises a sequence that is complementary to the target nucleic acid. In some instances, complexing of the gRNA “triggers” a nuclease activity of the CRISPR/nuclease gRNA complex. In preferred instances, “triggering” of a nuclease activity of the CRISPR/nuclease gRNA complex also requires a presence of a target nucleic acid. A target nucleic acid is one that hybridizes to a direct repeat with sufficient homology to trigger nuclease activation (e.g., greater than 80%).

A guide nucleic acid can comprise one or more direct repeat sequences, which can form secondary and/or tertiary stem-loop structures. In general, a “direct repeat sequence” includes any sequence that has sufficient sequence to promote formation of a CRISPR complex, wherein the complex comprises a nucleic acid-guided nuclease and a guide nucleic acid. Sufficient structure within the direct repeat sequence to promote formation of a CRISPR complex may include a degree of complementarity along the length of two sequence regions within the scaffold sequence, such as one or two sequence regions involved in forming a secondary structure. In some aspects, the complexes of the disclosure were identified based on putative and/or iterative analysis for identifying potential enzyme sequences that had a likelihood of forming a complex with potential gRNAs. The present disclosure implemented a proximal cut-off for the relative distance between the CRISPR arrays, which contains the direct repeat sequence information that can be incorporated into a gRNA sequence, to that of the putative CRISPR sequence on genomic or assembled metagenomics sequence input to be considered a candidate fit for additional testing. As a result, the present disclosure identified predicted CRISPR array sequence information accompanying every single candidate from the candidate pool. In some cases, sub-categories were identified bases on CRISPR sequences known in the art (e.g. Cas13a or Cas13b). The present disclosure designed, based on this CRISPR array sequence proximity, two gRNAs for each candidate where the spacer sequence was either at the 3′-end or the 5′-end of the direct repeats sequence.

A guide nucleic acid can be compatible with a nucleic acid-guided nuclease when the two elements can form a functional CRISPR nuclease complex capable of cleaving a nucleic acid sequence specifically (i.e., targeted activity) and/or non-specifically (i.e., collateral activity). In some instances, a compatible scaffold sequence for a compatible guide nucleic acid can be found by scanning sequences adjacent to a native nucleic acid-guided nuclease loci. For example, native nucleic acid-guided nucleases can be encoded on a genome within proximity to a corresponding compatible direct repeat sequence. Alternatively, a gRNA sequence may be identified, e.g., based on an optimal alignment of a nucleic acid sequence for forming a secondary structure that could complex with a CRISPR nuclease. It may be determined by any suitable alignment algorithm, and may further account for secondary structures, such as self-complementarity within either the one or more sequence regions forming a secondary structure. In some embodiments, the degree of complementarity between the one or two sequence regions along the length of the shorter of the two when optimally aligned is about or more than about 25%, 30%, 40%, 50%, 60%, 70%, 80%, 90%, 95%, 97.5%, 99%, or higher. In some embodiments, at least one of the two sequence regions is about or more than about 5, 6, 7, 8, 9, 10, 11, 12, 13, 14, 15, 16, 17, 18, 19, 20, 25, 30, 40, 50, or more nucleotides in length.

A guide nucleic acid can comprise a spacer sequence. A spacer sequence is a polynucleotide sequence having sufficient complementarity with a target polynucleotide sequence to hybridize with the target sequence. The degree of complementarity between a spacer sequence and its corresponding target sequence, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences. In some embodiments, a spacer sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a spacer sequence is less than about 75, 50, 45, 40, 35, 30, 25, 20 nucleotides in length. Preferably the spacer sequence is 10-40 nucleotides long. The spacer sequence can be 15-25 nucleotides in length. The spacer sequence can be ˜15 nucleotides in length. The spacer sequence can be 16 nucleotides in length. The spacer sequence can be 17 nucleotides in length. The spacer sequence can be 18 nucleotides in length. The spacer sequence can be 19 nucleotides in length. The spacer sequence can be 20 nucleotides in length. Non-limiting examples of putative spacer sequences considered by the invention include SEQ ID NOs:374-466. Certain exemplary gRNAs described herein are tested with specific spacer sequences (e.g., FLU A target), see SEQ ID NOs 94-279. It is to be understood however, that one of ordinary skill in the art, looking at certain tested gRNAs is readily able to identify the direct repeat sequence and distinguish it from the spacer sequence.

In some instances, a gRNA complexed with a compatible nucleic acid-guided nuclease can hybridize with a target sequence, thereby directing the nuclease to indiscriminately cleave all surrounding nucleic acid sequences, i.e., collateral activity. In these cases, the CRISPR nuclease can cleave not only the target sequence, but it may indiscriminately cleave its surrounding nucleic acids despite of their sequence. In other instances, a gRNA complexed with a compatible nucleic acid-guided nuclease can hybridize with a target sequence, thereby directing the nuclease to cleave target sequence, i.e., specific target activity. In certain cases, hybridization effectively “directs” sequence-specific binding of a complexed nucleic acid-guided nuclease to a target sequence. In these cases, the CRISPR nuclease may preferably cleave the target sequence.

A direct repeat sequence of a subject guide nucleic acid can comprise a secondary structure and/or a tertiary structure. In some cases, binding kinetics of a guide nucleic acid to a nucleic acid-guided nuclease is determined in part by secondary structures within the direct repeat sequence. In some cases, binding kinetics of a guide nucleic acid to a nucleic acid-guided nuclease is determined in part by nucleic acid sequence with the scaffold sequence. In CRISPR-based diagnostics, quantification through comparison with a standard curve can be achieved within the picomolar-to-micromolar range (10⁻¹² M-10⁻⁶ M), where CRISPR-based collateral-cleavage activity correlates with target concentration.

In some aspects, the invention provides a CRISPR nuclease that complexes with a guide nucleic acid comprising a direct repeat sequence. In certain cases, the disclosure contemplates certain complexes, including the CRISPR nuclease/direct repeat pair of SEQ ID NO:1/SEQ ID NO: 374 (JW13101); the CRISPR nuclease/direct repeat pair of SEQ ID NO:2/SEQ ID NO: 375 (JW13102); the CRISPR nuclease/direct repeat pair of SEQ ID NO:3/SEQ ID NO: 376 (JW13103); the CRISPR nuclease/direct repeat pair of SEQ ID NO:4/SEQ ID NO: 377 (JW13104); the CRISPR nuclease/direct repeat pair of SEQ ID NO:5/SEQ ID NO: 378 (JW13105); the CRISPR nuclease/direct repeat pair of SEQ ID NO:6/SEQ ID NO: 379 (JW13106); the CRISPR nuclease/direct repeat pair of SEQ ID NO:7/SEQ ID NO: 380 (JW13107); the CRISPR nuclease/direct repeat pair of SEQ ID NO:8/SEQ ID NO: 381 (JW13108); the CRISPR nuclease/direct repeat pair of SEQ ID NO:9/SEQ ID NO: 382 (JW13110); the CRISPR nuclease/direct repeat pair of SEQ ID NO: 10/SEQ ID NO: 383 (JW13112); the CRISPR nuclease/direct repeat pair of SEQ ID NO: 11/SEQ ID NO: 384 (JW13113); the CRISPR nuclease/direct repeat pair of SEQ ID NO: 12/SEQ ID NO: 385 (JW13114); the CRISPR nuclease/direct repeat pair of SEQ ID NO: 13/SEQ ID NO: 386 (JW13115); the CRISPR nuclease/direct repeat pair of SEQ ID NO: 14/SEQ ID NO: 387 (JW13116); the CRISPR nuclease/direct repeat pair of SEQ ID NO: 15/SEQ ID NO: 388 (JW13117); the CRISPR nuclease/direct repeat pair of SEQ ID NO: 16/SEQ ID NO: 389 (JW13118); the CRISPR nuclease/direct repeat pair of SEQ ID NO:17/SEQ ID NO: 390 (JW13119); the CRISPR nuclease/direct repeat pair of SEQ ID NO: 18/SEQ ID NO: 391 (JW13120); the CRISPR nuclease/direct repeat pair of SEQ ID NO: 19/SEQ ID NO: 392 (JW13121); the CRISPR nuclease/direct repeat pair of SEQ ID NO:20/SEQ ID NO: 393 (JW13122); the CRISPR nuclease/direct repeat pair of SEQ ID NO: 21/SEQ ID NO: 394 (JW13123); the CRISPR nuclease/direct repeat pair of SEQ ID NO:22/SEQ ID NO: 395 (JW13124); the CRISPR nuclease/direct repeat pair of SEQ ID NO:23/SEQ ID NO: 396 (JW13125); the CRISPR nuclease/direct repeat pair of SEQ ID NO:24/SEQ ID NO: 397 (JW13126); the CRISPR nuclease/direct repeat pair of SEQ ID NO:25/SEQ ID NO: 398 (JW13127); the CRISPR nuclease/direct repeat pair of SEQ ID NO:26/SEQ ID NO: 399 (JW13128); the CRISPR nuclease/direct repeat pair of SEQ ID NO:27/SEQ ID NO: 400 (JW13129); the CRISPR nuclease/direct repeat pair of SEQ ID NO:28/SEQ ID NO: 401 (JW13130); the CRISPR nuclease/direct repeat pair of SEQ ID NO:29/SEQ ID NO: 402 (JW13131); the CRISPR nuclease/direct repeat pair of SEQ ID NO:30/SEQ ID NO: 403 (JW13132); the CRISPR nuclease/direct repeat pair of SEQ ID NO:31/SEQ ID NO: 404 (JW13133); the CRISPR nuclease/direct repeat pair of SEQ ID NO:32/SEQ ID NO: 405 (JW13134); the CRISPR nuclease/direct repeat pair of SEQ ID NO:33/SEQ ID NO: 406 (JW13135); the CRISPR nuclease/direct repeat pair of SEQ ID NO:34/SEQ ID NO: 407 (JW13136); the CRISPR nuclease/direct repeat pair of SEQ ID NO:35/SEQ ID NO: 408 (JW13137); the CRISPR nuclease/direct repeat pair of SEQ ID NO:36/SEQ ID NO: 409 (JW13138); the CRISPR nuclease/direct repeat pair of SEQ ID NO:37/SEQ ID NO: 410 (JW13139); the CRISPR nuclease/direct repeat pair of SEQ ID NO:38/SEQ ID NO: 411 (JW13140); the CRISPR nuclease/direct repeat pair of SEQ ID NO:39/SEQ ID NO: 412 (JW13141); the CRISPR nuclease/direct repeat pair of SEQ ID NO:40/SEQ ID NO: 413 (JW13142); the CRISPR nuclease/direct repeat pair of SEQ ID NO:41/SEQ ID NO: 414 (JW13143); the CRISPR nuclease/direct repeat pair of SEQ ID NO:42/SEQ ID NO: 415 (JW13144); the CRISPR nuclease/direct repeat pair of SEQ ID NO:43/SEQ ID NO: 416 (JW13145); the CRISPR nuclease/direct repeat pair of SEQ ID NO:44/SEQ ID NO: 417 (JW13146); the CRISPR nuclease/direct repeat pair of SEQ ID NO:45/SEQ ID NO: 418 (JW13147); the CRISPR nuclease/direct repeat pair of SEQ ID NO:46/SEQ ID NO: 419 (JW13148); the CRISPR nuclease/direct repeat pair of SEQ ID NO:47/SEQ ID NO: 420 (JW13149); the CRISPR nuclease/direct repeat pair of SEQ ID NO:48/SEQ ID NO: 421 (JW13150); the CRISPR nuclease/direct repeat pair of SEQ ID NO:49/SEQ ID NO: 422 (JW13153); the CRISPR nuclease/direct repeat pair of SEQ ID NO:50/SEQ ID NO: 423 (JW13154); the CRISPR nuclease/direct repeat pair of SEQ ID NO:51/SEQ ID NO: 424 (JW13155); the CRISPR nuclease/direct repeat pair of SEQ ID NO:52/SEQ ID NO: 425 (JW13156); the CRISPR nuclease/direct repeat pair of SEQ ID NO:53/SEQ ID NO: 426 (JW13157); the CRISPR nuclease/direct repeat pair of SEQ ID NO:54/SEQ ID NO: 427 (JW13158); the CRISPR nuclease/direct repeat pair of SEQ ID NO:55/SEQ ID NO: 428 (JW13159); the CRISPR nuclease/direct repeat pair of SEQ ID NO:56/SEQ ID NO: 429 (JW13160); the CRISPR nuclease/direct repeat pair of SEQ ID NO:57/SEQ ID NO: 430 (JW13161); the CRISPR nuclease/direct repeat pair of SEQ ID NO:58/SEQ ID NO: 431 (JW13162); the CRISPR nuclease/direct repeat pair of SEQ ID NO:59/SEQ ID NO: 432 (JW13163); the CRISPR nuclease/direct repeat pair of SEQ ID NO:60/SEQ ID NO: 433 (JW13164); the CRISPR nuclease/direct repeat pair of SEQ ID NO:61/SEQ ID NO: 434 (JW13165); the CRISPR nuclease/direct repeat pair of SEQ ID NO:62/SEQ ID NO: 435 (JW13166); the CRISPR nuclease/direct repeat pair of SEQ ID NO:63/SEQ ID NO: 436 (JW13167); the CRISPR nuclease/direct repeat pair of SEQ ID NO:64/SEQ ID NO: 437 (JW13168); the CRISPR nuclease/direct repeat pair of SEQ ID NO:65/SEQ ID NO: 438 (JW13169); the CRISPR nuclease/direct repeat pair of SEQ ID NO:66/SEQ ID NO: 439 (JW13170); the CRISPR nuclease/direct repeat pair of SEQ ID NO:67/SEQ ID NO: 440 (JW13171); the CRISPR nuclease/direct repeat pair of SEQ ID NO:68/SEQ ID NO: 441 (JW13172); the CRISPR nuclease/direct repeat pair of SEQ ID NO: 69/SEQ ID NO: 442 (JW13173); the CRISPR nuclease/direct repeat pair of SEQ ID NO:70/SEQ ID NO: 443 (JW13174); the CRISPR nuclease/direct repeat pair of SEQ ID NO:71/SEQ ID NO: 444 (JW13175); the CRISPR nuclease/direct repeat pair of SEQ ID NO:72/SEQ ID NO: 445 (JW13176); the CRISPR nuclease/direct repeat pair of SEQ ID NO:73/SEQ ID NO: 446 (JW13177); the CRISPR nuclease/direct repeat pair of SEQ ID NO:74/SEQ ID NO: 447 (JW13178); the CRISPR nuclease/direct repeat pair of SEQ ID NO:75/SEQ ID NO: 448 (JW13179); the CRISPR nuclease/direct repeat pair of SEQ ID NO:76/SEQ ID NO: 449 (JW13180); the CRISPR nuclease/direct repeat pair of SEQ ID NO: 77/SEQ ID NO: 450 (JW13181); the CRISPR nuclease/direct repeat pair of SEQ ID NO:78/SEQ ID NO: 451 (JW13182); the CRISPR nuclease/direct repeat pair of SEQ ID NO:79/SEQ ID NO: 452 (JW13183); the CRISPR nuclease/direct repeat pair of SEQ ID NO:80/SEQ ID NO: 453 (JW13184); the CRISPR nuclease/direct repeat pair of SEQ ID NO:81/SEQ ID NO: 454 (JW13185); the CRISPR nuclease/direct repeat pair of SEQ ID NO:82/SEQ ID NO: 455 (JW13186); the CRISPR nuclease/direct repeat pair of SEQ ID NO:83/SEQ ID NO: 456 (JW13187); the CRISPR nuclease/direct repeat pair of SEQ ID NO:84/SEQ ID NO: 457 (JW13188); the CRISPR nuclease/direct repeat pair of SEQ ID NO:85/SEQ ID NO: 458 (JW13189); the CRISPR nuclease/direct repeat pair of SEQ ID NO:86/SEQ ID NO: 459 (JW13190); the CRISPR nuclease/direct repeat pair of SEQ ID NO:87/SEQ ID NO: 460 (JW13191); the CRISPR nuclease/direct repeat pair of SEQ ID NO:88/SEQ ID NO: 461 (JW13192); the CRISPR nuclease/direct repeat pair of SEQ ID NO:89/SEQ ID NO: 462 (JW13193); the CRISPR nuclease/direct repeat pair of SEQ ID NO:90/SEQ ID NO: 463 (JW13194); the CRISPR nuclease/direct repeat pair of SEQ ID NO:91/SEQ ID NO: 464 (JW13195); the CRISPR nuclease/direct repeat pair of SEQ ID NO:92/SEQ ID NO: 465 (JW13196); and/or the CRISPR nuclease/direct repeat pair of SEQ ID NO:93/SEQ ID NO: 466 (JW13197).

In some aspects, the invention provides a CRISPR nuclease that complexes with a guide nucleic acid comprising a direct repeat sequence and a spacer designed to hybridize (i.e., “target”) a specific target region of, e.g., a microorganism. In certain cases, the disclosure contemplates certain complexes, including the CRISPR nuclease/direct repeat pair of CRISPR nuclease/gRNA pair of SEQ ID NO:1/SEQ ID NO: 94 or the CRISPR nuclease/gRNA pair of SEQ ID NO:1/SEQ ID NO: 117 (JW13101); the CRISPR nuclease/gRNA pair of SEQ ID NO:2/SEQ ID NO: 95 or the CRISPR nuclease/gRNA pair of SEQ ID NO:2/SEQ ID NO: 118 (JW13102); the CRISPR nuclease/gRNA pair of SEQ ID NO:3/SEQ ID NO: 96 or the CRISPR nuclease/gRNA pair of SEQ ID NO:3/SEQ ID NO: 119 (JW13103); the CRISPR nuclease/gRNA pair of SEQ ID NO:4/SEQ ID NO: 97 or the CRISPR nuclease/gRNA pair of SEQ ID NO:4/SEQ ID NO: 120 (JW13104); the CRISPR nuclease/gRNA pair of SEQ ID NO:5/SEQ ID NO: 98 or the CRISPR nuclease/gRNA pair of SEQ ID NO:5/SEQ ID NO: 121 (JW13105); the CRISPR nuclease/gRNA pair of SEQ ID NO:6/SEQ ID NO: 99 or the CRISPR nuclease/gRNA pair of SEQ ID NO:6/SEQ ID NO: 122 (JW13106); the CRISPR nuclease/gRNA pair of SEQ ID NO:7/SEQ ID NO: 100 or the CRISPR nuclease/gRNA pair of SEQ ID NO:7/SEQ ID NO: 123 (JW13107); the CRISPR nuclease/gRNA pair of SEQ ID NO:8/SEQ ID NO: 101 or the CRISPR nuclease/gRNA pair of SEQ ID NO:8/SEQ ID NO: 124 (JW13108); the CRISPR nuclease/gRNA pair of SEQ ID NO:9/SEQ ID NO: 102 or the CRISPR nuclease/gRNA pair of SEQ ID NO:9/SEQ ID NO: 125 (JW13110); the CRISPR nuclease/gRNA pair of SEQ ID NO: 10/SEQ ID NO: 103 or the CRISPR nuclease/gRNA pair of SEQ ID NO: 10/SEQ ID NO: 126 (JW13112); the CRISPR nuclease/gRNA pair of SEQ ID NO:11/SEQ ID NO: 104 or the CRISPR nuclease/gRNA pair of SEQ ID NO: 11/SEQ ID NO: 127 (JW13113); the CRISPR nuclease/gRNA pair of SEQ ID NO:12/SEQ ID NO: 105 or the CRISPR nuclease/gRNA pair of SEQ ID NO:12/SEQ ID NO: 128 (JW13114); the CRISPR nuclease/gRNA pair of SEQ ID NO: 13/SEQ ID NO: 106 or the CRISPR nuclease/gRNA pair of SEQ ID NO: 13/SEQ ID NO: 129 (JW13115); the CRISPR nuclease/gRNA pair of SEQ ID NO: 14/SEQ ID NO: 107 or the CRISPR nuclease/gRNA pair of SEQ ID NO: 14/SEQ ID NO: 130 (JW13116); the CRISPR nuclease/gRNA pair of SEQ ID NO:15/SEQ ID NO: 108 or the CRISPR nuclease/gRNA pair of SEQ ID NO:15/SEQ ID NO: 131 (JW13117); the CRISPR nuclease/gRNA pair of SEQ ID NO: 16/SEQ ID NO: 109 or the CRISPR nuclease/gRNA pair of SEQ ID NO:16/SEQ ID NO: 132 (JW13118); the CRISPR nuclease/gRNA pair of SEQ ID NO: 17/SEQ ID NO: 110 or the CRISPR nuclease/gRNA pair of SEQ ID NO: 17/SEQ ID NO: 133 (JW13119); the CRISPR nuclease/gRNA pair of SEQ ID NO:18/SEQ ID NO: 111 or the CRISPR nuclease/gRNA pair of SEQ ID NO:18/SEQ ID NO: 134 (JW13120); the CRISPR nuclease/gRNA pair of SEQ ID NO: 19/SEQ ID NO: 112 or the CRISPR nuclease/gRNA pair of SEQ ID NO: 19/SEQ ID NO: 135 (JW13121); the CRISPR nuclease/gRNA pair of SEQ ID NO:20/SEQ ID NO: 113 or the CRISPR nuclease/gRNA pair of SEQ ID NO:20/SEQ ID NO: 136 (JW13122); the CRISPR nuclease/gRNA pair of SEQ ID NO:21/SEQ ID NO: 114 or the CRISPR nuclease/gRNA pair of SEQ ID NO:21/SEQ ID NO: 137 (JW13123); the CRISPR nuclease/gRNA pair of SEQ ID NO:22/SEQ ID NO: 115 or the CRISPR nuclease/gRNA pair of SEQ ID NO:22/SEQ ID NO: 138 (JW13124); the CRISPR nuclease/gRNA pair of SEQ ID NO:23/SEQ ID NO: 116 or the CRISPR nuclease/gRNA pair of SEQ ID NO:23/SEQ ID NO: 139 (JW13125); the CRISPR nuclease/gRNA pair of SEQ ID NO:24/SEQ ID NO: 140 or the CRISPR nuclease/gRNA pair of SEQ ID NO:24/SEQ ID NO: 164 (JW13126); the CRISPR nuclease/gRNA pair of SEQ ID NO:25/SEQ ID NO: 141 or the CRISPR nuclease/gRNA pair of SEQ ID NO:25/SEQ ID NO: 165 (JW13127); the CRISPR nuclease/gRNA pair of SEQ ID NO:26/SEQ ID NO: 142 or the CRISPR nuclease/gRNA pair of SEQ ID NO:26/SEQ ID NO: 166 (JW13128); the CRISPR nuclease/gRNA pair of SEQ ID NO:27/SEQ ID NO: 143 or the CRISPR nuclease/gRNA pair of SEQ ID NO:27/SEQ ID NO: 167 (JW13129); the CRISPR nuclease/gRNA pair of SEQ ID NO:28/SEQ ID NO: 144 or the CRISPR nuclease/gRNA pair of SEQ ID NO:28/SEQ ID NO: 168 (JW13130); the CRISPR nuclease/gRNA pair of SEQ ID NO:29/SEQ ID NO: 145 or the CRISPR nuclease/gRNA pair of SEQ ID NO:29/SEQ ID NO: 169 (JW13131); the CRISPR nuclease/gRNA pair of SEQ ID NO:30/SEQ ID NO: 146 or the CRISPR nuclease/gRNA pair of SEQ ID NO:30/SEQ ID NO: 170 (JW13132); the CRISPR nuclease/gRNA pair of SEQ ID NO:31/SEQ ID NO: 147 or the CRISPR nuclease/gRNA pair of SEQ ID NO:31/SEQ ID NO: 171 (JW13133); the CRISPR nuclease/gRNA pair of SEQ ID NO:32/SEQ ID NO: 148 or the CRISPR nuclease/gRNA pair of SEQ ID NO:32/SEQ ID NO: 172 (JW13134); the CRISPR nuclease/gRNA pair of SEQ ID NO:33/SEQ ID NO: 149 or the CRISPR nuclease/gRNA pair of SEQ ID NO:33/SEQ ID NO: 173 (JW13135); the CRISPR nuclease/gRNA pair of SEQ ID NO:34/SEQ ID NO: 150 or the CRISPR nuclease/gRNA pair of SEQ ID NO:34/SEQ ID NO: 174 (JW13136); the CRISPR nuclease/gRNA pair of SEQ ID NO:35/SEQ ID NO: 151 or the CRISPR nuclease/gRNA pair of SEQ ID NO:35/SEQ ID NO: 175 (JW13137); the CRISPR nuclease/gRNA pair of SEQ ID NO:36/SEQ ID NO: 152 or the CRISPR nuclease/gRNA pair of SEQ ID NO:36/SEQ ID NO: 176 (JW13138); the CRISPR nuclease/gRNA pair of SEQ ID NO:37/SEQ ID NO: 153 or the CRISPR nuclease/gRNA pair of SEQ ID NO:37/SEQ ID NO: 177 (JW13139); the CRISPR nuclease/gRNA pair of SEQ ID NO:38/SEQ ID NO: 154 or the CRISPR nuclease/gRNA pair of SEQ ID NO:38/SEQ ID NO: 178 (JW13140); the CRISPR nuclease/gRNA pair of SEQ ID NO:39/SEQ ID NO: 155 or the CRISPR nuclease/gRNA pair of SEQ ID NO:39/SEQ ID NO: 179 (JW13141); the CRISPR nuclease/gRNA pair of SEQ ID NO:40/SEQ ID NO: 156 or the CRISPR nuclease/gRNA pair of SEQ ID NO:40/SEQ ID NO: 180 (JW13142); the CRISPR nuclease/gRNA pair of SEQ ID NO:41/SEQ ID NO: 157 or the CRISPR nuclease/gRNA pair of SEQ ID NO:41/SEQ ID NO: 181 (JW13143); the CRISPR nuclease/gRNA pair of SEQ ID NO:42/SEQ ID NO: 158 or the CRISPR nuclease/gRNA pair of SEQ ID NO:42/SEQ ID NO: 182 (JW13144); the CRISPR nuclease/gRNA pair of SEQ ID NO:43/SEQ ID NO: 159 or the CRISPR nuclease/gRNA pair of SEQ ID NO:43/SEQ ID NO: 183 (JW13145); the CRISPR nuclease/gRNA pair of SEQ ID NO:44/SEQ ID NO: 160 or the CRISPR nuclease/gRNA pair of SEQ ID NO:44/SEQ ID NO: 184 (JW13146); the CRISPR nuclease/gRNA pair of SEQ ID NO:45/SEQ ID NO: 161 or the CRISPR nuclease/gRNA pair of SEQ ID NO:45/SEQ ID NO: 185 (JW13147); the CRISPR nuclease/gRNA pair of SEQ ID NO:46/SEQ ID NO: 162 or the CRISPR nuclease/gRNA pair of SEQ ID NO:46/SEQ ID NO: 186 (JW13148); the CRISPR nuclease/gRNA pair of SEQ ID NO:47/SEQ ID NO: 163 or the CRISPR nuclease/gRNA pair of SEQ ID NO:47/SEQ ID NO: 187 (JW13149); the CRISPR nuclease/gRNA pair of SEQ ID NO:48/SEQ ID NO: 188 or the CRISPR nuclease/gRNA pair of SEQ ID NO:48/SEQ ID NO: 234 (JW13150); the CRISPR nuclease/gRNA pair of SEQ ID NO:49/SEQ ID NO: 189 or the CRISPR nuclease/gRNA pair of SEQ ID NO:49/SEQ ID NO: 235 (JW13153); the CRISPR nuclease/gRNA pair of SEQ ID NO:50/SEQ ID NO: 190 or the CRISPR nuclease/gRNA pair of SEQ ID NO:50/SEQ ID NO: 236 (JW13154); the CRISPR nuclease/gRNA pair of SEQ ID NO:51/SEQ ID NO: 191 or the CRISPR nuclease/gRNA pair of SEQ ID NO:51/SEQ ID NO: 237 (JW13155); the CRISPR nuclease/gRNA pair of SEQ ID NO:52/SEQ ID NO: 192 or the CRISPR nuclease/gRNA pair of SEQ ID NO:52/SEQ ID NO: 238 (JW13156); the CRISPR nuclease/gRNA pair of SEQ ID NO:53/SEQ ID NO: 193 or the CRISPR nuclease/gRNA pair of SEQ ID NO:53/SEQ ID NO: 239 (JW13157); the CRISPR nuclease/gRNA pair of SEQ ID NO:54/SEQ ID NO: 194 or the CRISPR nuclease/gRNA pair of SEQ ID NO:54/SEQ ID NO: 240 (JW13158); the CRISPR nuclease/gRNA pair of SEQ ID NO:55/SEQ ID NO: 195 or the CRISPR nuclease/gRNA pair of SEQ ID NO:55/SEQ ID NO: 241 (JW13159); the CRISPR nuclease/gRNA pair of SEQ ID NO:56/SEQ ID NO: 196 or the CRISPR nuclease/gRNA pair of SEQ ID NO:56/SEQ ID NO: 242 (JW13160); the CRISPR nuclease/gRNA pair of SEQ ID NO:57/SEQ ID NO: 197 or the CRISPR nuclease/gRNA pair of SEQ ID NO:57/SEQ ID NO: 243 (JW13161); the CRISPR nuclease/gRNA pair of SEQ ID NO:58/SEQ ID NO: 198 or the CRISPR nuclease/gRNA pair of SEQ ID NO:58/SEQ ID NO: 244 (JW13162); the CRISPR nuclease/gRNA pair of SEQ ID NO:59/SEQ ID NO: 199 or the CRISPR nuclease/gRNA pair of SEQ ID NO:59/SEQ ID NO: 245 (JW13163); the CRISPR nuclease/gRNA pair of SEQ ID NO:60/SEQ ID NO: 200 or the CRISPR nuclease/gRNA pair of SEQ ID NO:60/SEQ ID NO: 246 (JW13164); the CRISPR nuclease/gRNA pair of SEQ ID NO:61/SEQ ID NO: 201 or the CRISPR nuclease/gRNA pair of SEQ ID NO:61/SEQ ID NO: 247 (JW13165); the CRISPR nuclease/gRNA pair of SEQ ID NO:62/SEQ ID NO: 202 or the CRISPR nuclease/gRNA pair of SEQ ID NO:62/SEQ ID NO: 248 (JW13166); the CRISPR nuclease/gRNA pair of SEQ ID NO:63/SEQ ID NO: 203 or the CRISPR nuclease/gRNA pair of SEQ ID NO:63/SEQ ID NO: 249 (JW13167); the CRISPR nuclease/gRNA pair of SEQ ID NO:64/SEQ ID NO: 204 or the CRISPR nuclease/gRNA pair of SEQ ID NO:64/SEQ ID NO: 250 (JW13168); the CRISPR nuclease/gRNA pair of SEQ ID NO:65/SEQ ID NO: 205 or the CRISPR nuclease/gRNA pair of SEQ ID NO:65/SEQ ID NO: 251 (JW13169); the CRISPR nuclease/gRNA pair of SEQ ID NO:66/SEQ ID NO: 206 or the CRISPR nuclease/gRNA pair of SEQ ID NO:66/SEQ ID NO: 252 (JW13170); the CRISPR nuclease/gRNA pair of SEQ ID NO:67/SEQ ID NO: 207 or the CRISPR nuclease/gRNA pair of SEQ ID NO:67/SEQ ID NO: 253 (JW13171); the CRISPR nuclease/gRNA pair of SEQ ID NO:68/SEQ ID NO: 208 or the CRISPR nuclease/gRNA pair of SEQ ID NO:68/SEQ ID NO: 254 (JW13172); the CRISPR nuclease/gRNA pair of SEQ ID NO:69/SEQ ID NO: 209 or the CRISPR nuclease/gRNA pair of SEQ ID NO:69/SEQ ID NO: 255 (JW13173); the CRISPR nuclease/gRNA pair of SEQ ID NO:70/SEQ ID NO: 210 or the CRISPR nuclease/gRNA pair of SEQ ID NO:70/SEQ ID NO: 256 (JW13174); the CRISPR nuclease/gRNA pair of SEQ ID NO:71/SEQ ID NO: 211 or the CRISPR nuclease/gRNA pair of SEQ ID NO:71/SEQ ID NO: 257 (JW13175); the CRISPR nuclease/gRNA pair of SEQ ID NO:72/SEQ ID NO: 212 or the CRISPR nuclease/gRNA pair of SEQ ID NO:72/SEQ ID NO: 258 (JW13176); the CRISPR nuclease/gRNA pair of SEQ ID NO:73/SEQ ID NO: 213 or the CRISPR nuclease/gRNA pair of SEQ ID NO:73/SEQ ID NO: 259 (JW13177); the CRISPR nuclease/gRNA pair of SEQ ID NO:74/SEQ ID NO: 214 or the CRISPR nuclease/gRNA pair of SEQ ID NO:74/SEQ ID NO: 260 (JW13178); the CRISPR nuclease/gRNA pair of SEQ ID NO:75/SEQ ID NO: 215 or the CRISPR nuclease/gRNA pair of SEQ ID NO:75/SEQ ID NO: 261 (JW13179); the CRISPR nuclease/gRNA pair of SEQ ID NO:76/SEQ ID NO: 216 or the CRISPR nuclease/gRNA pair of SEQ ID NO:76/SEQ ID NO: 262 (JW13180); the CRISPR nuclease/gRNA pair of SEQ ID NO:77/SEQ ID NO: 217 or the CRISPR nuclease/gRNA pair of SEQ ID NO:77/SEQ ID NO: 263 (JW13181); the CRISPR nuclease/gRNA pair of SEQ ID NO:78/SEQ ID NO: 218 or the CRISPR nuclease/gRNA pair of SEQ ID NO:78/SEQ ID NO: 264 (JW13182); the CRISPR nuclease/gRNA pair of SEQ ID NO:79/SEQ ID NO: 219 or the CRISPR nuclease/gRNA pair of SEQ ID NO:79/SEQ ID NO: 265 (JW13183); the CRISPR nuclease/gRNA pair of SEQ ID NO:80/SEQ ID NO: 220 or the CRISPR nuclease/gRNA pair of SEQ ID NO:80/SEQ ID NO: 266 (JW13184); the CRISPR nuclease/gRNA pair of SEQ ID NO:81/SEQ ID NO: 221 or the CRISPR nuclease/gRNA pair of SEQ ID NO:81/SEQ ID NO: 267 (JW13185); the CRISPR nuclease/gRNA pair of SEQ ID NO:82/SEQ ID NO: 222 or the CRISPR nuclease/gRNA pair of SEQ ID NO:82/SEQ ID NO: 268 (JW13186); the CRISPR nuclease/gRNA pair of SEQ ID NO:83/SEQ ID NO: 223 or the CRISPR nuclease/gRNA pair of SEQ ID NO:83/SEQ ID NO: 269 (JW13187); the CRISPR nuclease/gRNA pair of SEQ ID NO:84/SEQ ID NO: 224 or the CRISPR nuclease/gRNA pair of SEQ ID NO:84/SEQ ID NO: 270 (JW13188); the CRISPR nuclease/gRNA pair of SEQ ID NO:85/SEQ ID NO: 225 or the CRISPR nuclease/gRNA pair of SEQ ID NO:85/SEQ ID NO: 271 (JW13189); the CRISPR nuclease/gRNA pair of SEQ ID NO:86/SEQ ID NO: 226 or the CRISPR nuclease/gRNA pair of SEQ ID NO:86/SEQ ID NO: 272 (JW13190); the CRISPR nuclease/gRNA pair of SEQ ID NO:87/SEQ ID NO: 227 or the CRISPR nuclease/gRNA pair of SEQ ID NO:87/SEQ ID NO: 273 (JW13191); the CRISPR nuclease/gRNA pair of SEQ ID NO:88/SEQ ID NO: 228 or the CRISPR nuclease/gRNA pair of SEQ ID NO:88/SEQ ID NO: 274 (JW13192); the CRISPR nuclease/gRNA pair of SEQ ID NO:89/SEQ ID NO: 229 or the CRISPR nuclease/gRNA pair of SEQ ID NO:89/SEQ ID NO: 275 (JW13193); the CRISPR nuclease/gRNA pair of SEQ ID NO:90/SEQ ID NO: 230 or the CRISPR nuclease/gRNA pair of SEQ ID NO:90/SEQ ID NO: 276 (JW13194); the CRISPR nuclease/gRNA pair of SEQ ID NO:91/SEQ ID NO: 231 or the CRISPR nuclease/gRNA pair of SEQ ID NO:91/SEQ ID NO: 277 (JW13195); the CRISPR nuclease/gRNA pair of SEQ ID NO:92/SEQ ID NO: 232 or the CRISPR nuclease/gRNA pair of SEQ ID NO:92/SEQ ID NO: 278 (JW13196); and/or the CRISPR nuclease/gRNA pair of SEQ ID NO:93/SEQ ID NO: 233 or the CRISPR nuclease/gRNA pair of SEQ ID NO:93/SEQ ID NO: 279 (JW13197). In particular instances, the CRISPR nuclease/gRNA pair is SEQ ID NO:22/SEQ ID NO: 115 or the CRISPR nuclease/gRNA pair of SEQ ID NO:22/SEQ ID NO: 138. A subject nucleic acid-guided nuclease capable of complexing with a guide nucleic acid can be referred to as a nucleic acid-guided nuclease that is compatible with the guide nucleic acid. Likewise, a guide nucleic acid capable of complexing with a nucleic acid-guided nuclease can be referred to as a guide nucleic acid that is compatible with the nucleic acid-guided nucleases.

In cases where the guide nucleic acid comprises RNA, the RNA guide nucleic acid can be encoded by a DNA sequence on a polynucleotide molecule such as a plasmid, linear construct, or editing cassette as disclosed herein. See, e.g., SEQ ID Nos: 280-372. A guide nucleic acid can comprise modified of non-naturally occurring nucleotides. A guide nucleic acid can comprise deoxyribonucleotides (DNA). A guide nucleic acid can comprise both DNA and RNA. A guide nucleic acid can comprise modified of non-naturally occurring nucleotides.

### Methods

All of the functionalities described in connection with the devices, systems, and processes described herein are intended to be applicable to detection of at least one target microorganism from a sample. The sample can comprise a mixture of viral nucleic acids, mammalian nucleic acids, and bacterial nucleic acids.

In some aspects the disclosure provides a method of detecting a target nucleic acid, the method comprising: contacting a sample with a nucleic acid-guided nuclease system comprising: a nucleic acid-guided CRISPR nuclease having at least 90% identity to SEQ ID NO: 1-93, or a nucleic acid molecule encoding the nucleic acid-guided CRISPR nuclease; a guide nucleic acid for complexing with the nucleic acid-guided CRISPR nuclease, or a nucleic acid molecule encoding the guide nucleic acid; and allowing the target nucleic acid to activate the nucleic acid-guided CRISPR nuclease system to cleave a detectable substrate.

In certain implementations of this method, the reaction mixtures and enzyme systems described herein are configured for the detection of one or more of SARS-CoV-2, influenza A, influenza B, Human Respiratory Syncytial Virus (RSV). In other cases, the reaction mixtures and enzyme systems described herein are configured for the detection of one or more of SARS-CoV-2, influenza A, influenza B, Human Respiratory Syncytial Virus (RSV), adenovirus, coronavirus 229E, coronavirus HKU1, coronavirus NL63, human metapneumovirus, human rhinovirus/enterovirus, parainfluenza virus 1, parainfluenza virus 2, parainfluenza virus 3, parainfluenza virus 4.

### Reaction Mixtures, Enzyme Systems, and Substrates

In some aspects, the disclosure provides reaction mixtures and enzyme systems that provide functional complexes with nuclease activity. A CRISPR reaction mixture typically contains a nucleic acid-guided nuclease (CRISPR nuclease), a guide nucleic acid (gRNA), a substrate for the complex, and optionally, depending on the enzyme, any co-factors (e.g., divalent cations such as Mg²⁺) that may be required for activation of the enzyme. The reaction mixture may comprise a substrate for the enzyme, such as an RNA or DNA molecule. The substrate may be labeled with a detectable moiety. The reaction mixture may have activity in a buffer that supports optimal pH and ionic conditions for the reaction, but in some cases the reaction mixture may be active in solutions that do not necessarily provide buffering. In preferred cases, the nucleic acid-guided CRISPR nuclease has trans-cleavage (i.e., collateral) activity, and indiscriminately cleaves nucleic acid substrates in the solution.

In some instances, the substrate for the complex is functionally connected to a detectable moiety. The detectable moiety can be conjugated onto any nucleotide substrate of suitable length so long as it does not inhibit cleavage of the nucleic acid by an activated CRISPR enzyme complex. A nucleic acid substrate for the complex can be, for e.g., 5, 10, 15, 20, 25, 30, 40, 45, 50, 55, 60, 65, 70, 75, 80, 85, 90, 95, 100, 105, 110, 115, 120, 125, 130, 140, 145, 150, 155, 160, 165, 170, 175, 180, 185, 190, 195, 200 nucleotides in length or larger. In some embodiments, the 5′ end of a nucleotide sequence of the substrate is conjugated to one or more (e.g., one, two, three, four, five, or more) detectable moieties. In some embodiments, the 3′ end of a nucleotide sequence of the substrate is conjugated to one or more (e.g., one, two, three, four, five, or more) detectable moieties. In some embodiments, the detectable moiety is a fluorophore complexed with a quencher for the fluorophore. In some embodiments, the detectable moiety itself is conjugated to the signal-producing agent. In some embodiments, the detectable moiety is a molecule that can bind a binding partner and the binding partner is linked to a signal producing agent. For example, in some embodiments, the detectable moiety is biotin and the binding partner is streptavidin or vice-versa. The signal-producing agent can be any agent that produces quantifiable signal, including but not limited to, chemiluminescence, color, or fluorescence. Non-limiting examples of signal-producing agents include an enzyme, a fluorescent molecule, or the like. In some embodiments, the substrate is a ribonucleic acid molecule (RNA) complexed with biotin. In some embodiments, the detectable label is biotin and the method comprises contacting the detectable label with a streptavidin-linked signal producing agent. In some instances, the reaction mixture or the system may comprise a general RNAase inhibitor to minimize signals that are not specifically generated by activation of the CRISPR enzyme.

The substrate for the complex can be present in the reaction mixture in any suitable concentration for detection by the CRISPR enzyme. In some cases, the substrate is present in the reaction mix in a concentration ranging from 10 nM to 1,000 nM, from 20 nM to 1,000 nM, from 30 nM to 1,000 nM, from 40 nM to 1,000 nM, from 50 nM to 1,000 nM, from 60 nM to 1,000 nM, from 70 nM to 1,000 nM, from 80 nM to 1,000 nM, from 90 nM to 1,000 nM, from 100 nM to 1,000 nM, from 110 nM to 1,000 nM, from 120 nM to 1,000 nM, from 130 nM to 1,000 nM, from 140 nM to 1,000 nM, from 150 nM to 1,000 nM, from 160 nM to 1,000 nM, from 170 nM to 1,000 nM, from 180 nM to 1,000 nM, from 190 nM to 1,000 nM, from 200 nM to 1,000 nM, from 210 nM to 1,000 nM, from 220 nM to 1,000 nM, from 230 nM to 1,000 nM, from 240 nM to 1,000 nM, from 250 nM to 1,000 nM, 10 nM to 750 nM, from 20 nM to 750 nM, from 30 nM to 750 nM, from 40 nM to 750 nM, from 50 nM to 750 nM, from 60 nM to 750 nM, from 70 nM to 750 nM, from 80 nM to 750 nM, from 90 nM to 750 nM, from 100 nM to 750 nM, from 110 nM to 750 nM, from 120 nM to 750 nM, from 130 nM to 750 nM, from 140 nM to 750 nM, from 150 nM to 750 nM, from 160 nM to 750 nM, from 170 nM to 750 nM, from 180 nM to 750 nM, from 190 nM to 750 nM, from 200 nM to 750 nM, from 210 nM to 750 nM, from 220 nM to 750 nM, from 230 nM to 750 nM, from 240 nM to 750 nM, from 250 nM to 750 nM, 10 nM to 500 nM, from 20 nM to 500 nM, from 30 nM to 500 nM, from 40 nM to 500 nM, from 50 nM to 500 nM, from 60 nM to 500 nM, from 70 nM to 500 nM, from 80 nM to 500 nM, from 90 nM to 500 nM, from 100 nM to 500 nM, from 110 nM to 500 nM, from 120 nM to 500 nM, from 130 nM to 500 nM, from 140 nM to 500 nM, from 150 nM to 500 nM, from 160 nM to 500 nM, from 170 nM to 500 nM, from 180 nM to 500 nM, from 190 nM to 500 nM, from 200 nM to 500 nM, from 210 nM to 500 nM, from 220 nM to 500 nM, from 230 nM to 500 nM, from 240 nM to 500 nM, from 250 nM to 500 nM, 10 nM to 450 nM, from 20 nM to 450 nM, from 30 nM to 450 nM, from 40 nM to 450 nM, from 50 nM to 450 nM, from 60 nM to 450 nM, from 70 nM to 450 nM, from 80 nM to 450 nM, from 90 nM to 450 nM, from 100 nM to 450 nM, from 110 nM to 450 nM, from 120 nM to 450 nM, from 130 nM to 450 nM, from 140 nM to 450 nM, from 150 nM to 450 nM, from 160 nM to 450 nM, from 170 nM to 450 nM, from 180 nM to 450 nM, from 190 nM to 450 nM, from 200 nM to 450 nM, from 210 nM to 450 nM, from 220 nM to 450 nM, from 230 nM to 450 nM, from 240 nM to 450 nM, from 250 nM to 450 nM, 10 nM to 400 nM, from 20 nM to 400 nM, from 30 nM to 400 nM, from 40 nM to 400 nM, from 50 nM to 400 nM, from 60 nM to 450 nM, from 70 nM to 450 nM, from 80 nM to 450 nM, from 90 nM to 450 nM, from 100 nM to 400 nM, from 110 nM to 400 nM, from 120 nM to 400 nM, from 130 nM to 400 nM, from 140 nM to 400 nM, from 150 nM to 400 nM, from 160 nM to 400 nM, from 170 nM to 400 nM, from 180 nM to 400 nM, from 190 nM to 400 nM, from 200 nM to 400 nM, from 210 nM to 400 nM, from 220 nM to 400 nM, from 230 nM to 400 nM, from 240 nM to 400 nM, from 250 nM to 400 nM.

In some cases, the substrate is present in the reaction mix in a concentration of no more than 10 nM, no more than 20 nM, no more than 30 nM, no more than 40 nM, no more than 50 nM, no more than 60 nM, no more than 70 nM, no more than 80 nM, no more than 90 nM, no more than 100 nM, no more than 110 nM, no more than 120 nM, no more than 130 nM, no more than 140 nM, no more than 150 nM, no more than 160 nM, no more than 170 nM, no more than 180 nM, no more than 190 nM, no more than 200 nM, no more than 210 nM, no more than 220 nM, no more than 230 nM, no more than 240 nM, no more than 250 nM, no more than 260 nM, no more than 270 nM, no more than 280 nM, no more than 290 nM, no more than 300 nM, no more than 310 nM, no more than 320 nM, no more than 330 nM, no more than 340 nM, no more than 350 nM, no more than 360 nM, no more than 370 nM, no more than 380 nM, no more than 390 nM, no more than 410 nM, no more than 420 nM, no more than 430 nM, no more than 440 nM, no more than 450 nM, no more than 460 nM, no more than 470 nM, no more than 480 nM, no more than 490 nM, no more than 500 nM, no more than 510 nM, no more than 520 nM, no more than 530 nM, no more than 540 nM, no more than 550 nM, no more than 560 nM, no more than 570 nM, no more than 580 nM, no more than 590 nM, no more than 600 nM, no more than 610 nM, no more than 620 nM, no more than 630 nM, no more than 640 nM, no more than 650 nM, no more than 660 nM, no more than 670 nM, no more than 680 nM, no more than 690 nM, no mora than 700 nM, no more than 710 nM, no more than 720 nM, no more than 730 nM, no more than 740 nM, no more than 750 nM, no more than 800 nM, no more than 850 nM, no more than 900 nM, no more than 950 nM, no more than 1,000 nM, no more than 2,000 nM, no more than 3,000 nM, no more than 4,000 nM, no more than 5,000 nM, no more than 6,000 nM, no more than 7,000 nM, no more than 8,000 nM, or another suitable amount.

In some embodiments a target sequence is present in the reaction mixture, whereby the target sequence activates cleavage of a substrate by the nucleic acid-guided CRISPR nuclease. In such instances the reaction mixture may be said to produce a “positive” result, or a positive identification of a presence of at least a minimum concentration of the target sequence.

Functional activity of the CRISPR enzyme/gRNA complex can be drive by an amount of gRNA in the reaction mixture available to form a complex with the CRISPR enzyme. In some cases, a gRNA can be present in the reaction mix in a concentration ranging from 10 ng/uL to 500 ng/uL, from 10 ng/uL to 490 ng/uL, from 10 ng/uL to 480 ng/uL, from 10 ng/uL to 470 ng/uL, from 10 ng/uL to 460 ng/uL, from 10 ng/uL to 450 ng/uL, from 10 ng/uL to 440 ng/uL, from 10 ng/uL to 430 ng/uL, from 10 ng/uL to 420 ng/uL, from 10 ng/uL to 410 ng/uL, from 10 ng/uL to 400 ng/uL, from 10 ng/uL to 390 ng/uL, from 10 ng/uL to 380 ng/uL, from 10 ng/uL to 370 ng/uL, from 10 ng/uL to 360 ng/uL, from 10 ng/uL to 350 ng/uL, from 10 ng/uL to 340 ng/uL, from 10 ng/uL to 330 ng/uL, from 10 ng/uL to 320 ng/uL, from 10 ng/uL to 310 ng/uL, from 10 ng/uL to 300 ng/uL, from 10 ng/uL to 290 ng/uL, from 10 ng/uL to 280 ng/uL, from 10 ng/uL to 270 ng/uL, from 10 ng/uL to 260 ng/uL, from 10 ng/uL to 250 ng/uL, from 10 ng/uL to 240 ng/uL, from 10 ng/uL to 230 ng/uL, from 10 ng/uL to 220 ng/uL, from 10 ng/uL to 210 ng/uL, from 10 ng/uL to 200 ng/uL, from 10 ng/uL to 190 ng/uL, from 10 ng/uL to 180 ng/uL, from 10 ng/uL to 170 ng/uL, from 10 ng/uL to 160 ng/uL, from 10 ng/uL to 150 ng/uL, from 10 ng/uL to 140 ng/uL, from 10 ng/uL to 130 ng/uL, from 10 ng/uL to 120 ng/uL, from 10 ng/uL to 110 ng/uL, from 10 ng/uL to 100 ng/uL.

In some instances the nucleic acid-guided CRISPR nuclease cleaves the substrate with a sensitivity of at least 20 nM, at least 21 nM, at least 22 nM, at least 23 nM, at least 24 nM, at least 25 nM, at least 26 nM, at least 27 nM, at least 28 nM, at least 29 nM, at least 30 nM, at least 31 nM, at least 32 nM, at least 33 nM, at least 34 nM, at least 35 nM, at least 36 nM, at least 37 nM, at least 38 nM, at least 39 nM, at least 40 nM, at least 41 nM, at least 42 nM, at least 43 nM, at least 44 nM, at least 45 nM, at least 46 nM, at least 47 nM, at least 48 nM, at least 49 nM, at least 50 nM, at least 51 nM, at least 52 nM, at least 53 nM, at least 54 nM, at least 55 nM, at least 56 nM, at least 57 nM, at least 58 nM, at least 59 nM, at least 60 nM, at least 61 nM, at least 62 nM, at least 63 nM, at least 64 nM, at least 65 nM, at least 66 nM, at least 67 nM, at least 68 nM, at least 69 nM, at least 70 nM, at least 71 nM, at least 72 nM, at least 73 nM, at least 74 nM, at least 75 nM, at least 76 nM, at least 77 nM, at least 78 nM, at least 79 nM, at least 80 nM, at least 81 nM, at least 82 nM, at least 83 nM, at least 84 nM, at least 85 nM, at least 86 nM, at least 87 nM, at least 88 nM, at least 89 nM, at least 90 nM, at least 91 nM, at least 92 nM, at least 93 nM, at least 94 nM, at least 95 nM, at least 96 nM, at least 97 nM, at least 98 nM, at least 99 nM, or at least 100 nM. In some instances, a CRISPR enzyme of the disclosure JW13124 (SEQ ID NO: 22) complexed with a compatible gRNA (e.g., a gRNA comprising a direct repeat of SEQ ID NO: 395) cleaves a nucleic acid sequence at least 5%, at least 10%, at least 15%, at least 20%, at least 25%, at least 30%, at least 35%, at least 40%, at least 45%, at least 50%, at least 55%, at least 60%, at least 65%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 155%, at least 160%, at least 165%, at least 165%, at least 170%, at least 175%, at least 180%, at least 185%, at least 190%, at least 195%, at least 200%, more efficiently than LwaCas13a when activated by binding of a target sequence.

In some instances, the reaction mixture further comprises one or more additional nucleic acid sequences that share a percent complementarity to at least a portion of a target or a non-target sequence. Upon binding to the target sequence (complementary to the spacer region of the gRNA), the gRNA/CRISPR complex will get activated and nuclease activity will ensue. Such nucleic acids can be present in the reaction mix in a concentration ranging from 10 ng/uL to 500 ng/uL, from 10 ng/uL to 490 ng/uL, from 10 ng/uL to 480 ng/uL, from 10 ng/uL to 470 ng/uL, from 10 ng/uL to 460 ng/uL, from 10 ng/uL to 450 ng/uL, from 10 ng/uL to 440 ng/uL, from 10 ng/uL to 430 ng/uL, from 10 ng/uL to 420 ng/uL, from 10 ng/uL to 410 ng/uL, from 10 ng/uL to 400 ng/uL, from 10 ng/uL to 390 ng/uL, from 10 ng/uL to 380 ng/uL, from 10 ng/uL to 370 ng/uL, from 10 ng/uL to 360 ng/uL, from 10 ng/uL to 350 ng/uL, from 10 ng/uL to 340 ng/uL, from 10 ng/uL to 330 ng/uL, from 10 ng/uL to 320 ng/uL, from 10 ng/uL to 310 ng/uL, from 10 ng/uL to 300 ng/uL, from 10 ng/uL to 290 ng/uL, from 10 ng/uL to 280 ng/uL, from 10 ng/uL to 270 ng/uL, from 10 ng/uL to 260 ng/uL, from 10 ng/uL to 250 ng/uL, from 10 ng/uL to 240 ng/uL, from 10 ng/uL to 230 ng/uL, from 10 ng/uL to 220 ng/uL, from 10 ng/uL to 210 ng/uL, from 10 ng/uL to 200 ng/uL, from 10 ng/uL to 190 ng/uL, from 10 ng/uL to 180 ng/uL, from 10 ng/uL to 170 ng/uL, from 10 ng/uL to 160 ng/uL, from 10 ng/uL to 150 ng/uL, from 10 ng/uL to 140 ng/uL, from 10 ng/uL to 130 ng/uL, from 10 ng/uL to 120 ng/uL, from 10 ng/uL to 110 ng/uL, from 10 ng/uL to 100 ng/uL.

### EMBODIMENTS

Embodiment 1. In some Embodiments, the disclosure provides a reaction mixture comprising: a nucleic acid-guided CRISPR nuclease (CRISPR nuclease) having at least 85% amino acid homology to any one of SEQ ID NOs:1-93; a guide nucleic acid (gRNA) for complexing with the nucleic acid-guided CRISPR nuclease, thereby providing a complex comprising a nucleic acid-guided CRISPR nuclease/gRNA pair, wherein the gRNA comprises a nucleic acid sequence, e.g., a gRNA sequence, and wherein the nucleic acid sequence comprises (i) a direct repeat sequence having at least 65%, at least 85%, at least 90%, or at least 95% nucleic acid homology to any one of SEQ ID NOs:374-466, and optionally (ii) a nucleic acid sequence having at least 85% nucleic acid homology to any one of SEQ ID NOs:94-279 (e.g., FluA target); and a substrate for the complex, optionally wherein the reaction mixture is in solution, further optionally wherein the solution comprises a buffer that supports optimal pH and ionic conditions for the reaction.

Embodiment 2. The reaction mixture of Embodiment 1, wherein the nucleic acid-guided CRISPR nuclease has at least 90% amino acid homology to any one of SEQ ID NOs:1-93.

Embodiment 3. The reaction mixture of any one of Embodiments 1 or 2, wherein the nucleic acid-guided CRISPR nuclease has at least 95% amino acid homology to any one of SEQ ID NOs:1-93.

Embodiment 4. The reaction mixture of any one of Embodiments 1-3, wherein the nucleic acid-guided CRISPR nuclease has at least 99% amino acid homology to any one of SEQ ID NOs:1-93.

Embodiment 5. The reaction mixture of any one of Embodiments 1-4, wherein the nucleic acid-guided CRISPR nuclease and the guide nucleic acid comprise a nucleic acid-guided CRISPR nuclease/gRNA pair selected from the group consisting of: the CRISPR nuclease/gRNA pair of SEQ ID NO:1/SEQ ID NO: 94 or the CRISPR nuclease/gRNA pair of SEQ ID NO:1/SEQ ID NO: 117; the CRISPR nuclease/gRNA pair of SEQ ID NO:2/SEQ ID NO: 95 or the CRISPR nuclease/gRNA pair of SEQ ID NO:2/SEQ ID NO: 118; the CRISPR nuclease/gRNA pair of SEQ ID NO:3/SEQ ID NO: 96 or the CRISPR nuclease/gRNA pair of SEQ ID NO:3/SEQ ID NO: 119; the CRISPR nuclease/gRNA pair of SEQ ID NO:4/SEQ ID NO: 97 or the CRISPR nuclease/gRNA pair of SEQ ID NO:4/SEQ ID NO: 120; the CRISPR nuclease/gRNA pair of SEQ ID NO:5/SEQ ID NO: 98 or the CRISPR nuclease/gRNA pair of SEQ ID NO:5/SEQ ID NO: 121; the CRISPR nuclease/gRNA pair of SEQ ID NO:6/SEQ ID NO: 99 or the CRISPR nuclease/gRNA pair of SEQ ID NO:6/SEQ ID NO: 122; the CRISPR nuclease/gRNA pair of SEQ ID NO:7/SEQ ID NO: 100 or the CRISPR nuclease/gRNA pair of SEQ ID NO:7/SEQ ID NO: 123; the CRISPR nuclease/gRNA pair of SEQ ID NO:8/SEQ ID NO: 101 or the CRISPR nuclease/gRNA pair of SEQ ID NO:8/SEQ ID NO: 124; the CRISPR nuclease/gRNA pair of SEQ ID NO:9/SEQ ID NO: 102 or the CRISPR nuclease/gRNA pair of SEQ ID NO:9/SEQ ID NO: 125; the CRISPR nuclease/gRNA pair of SEQ ID NO:10/SEQ ID NO: 103 or the CRISPR nuclease/gRNA pair of SEQ ID NO:10/SEQ ID NO: 126; the CRISPR nuclease/gRNA pair of SEQ ID NO: 11/SEQ ID NO: 104 or the CRISPR nuclease/gRNA pair of SEQ ID NO: 11/SEQ ID NO: 127; the CRISPR nuclease/gRNA pair of SEQ ID NO: 12/SEQ ID NO: 105 or the CRISPR nuclease/gRNA pair of SEQ ID NO:12/SEQ ID NO: 128; the CRISPR nuclease/gRNA pair of SEQ ID NO:13/SEQ ID NO: 106 or the CRISPR nuclease/gRNA pair of SEQ ID NO: 13/SEQ ID NO: 129; the CRISPR nuclease/gRNA pair of SEQ ID NO: 14/SEQ ID NO: 107 or the CRISPR nuclease/gRNA pair of SEQ ID NO: 14/SEQ ID NO: 130; the CRISPR nuclease/gRNA pair of SEQ ID NO: 15/SEQ ID NO: 108 or the CRISPR nuclease/gRNA pair of SEQ ID NO: 15/SEQ ID NO: 131; the CRISPR nuclease/gRNA pair of SEQ ID NO: 16/SEQ ID NO: 109 or the CRISPR nuclease/gRNA pair of SEQ ID NO: 16/SEQ ID NO: 132; the CRISPR nuclease/gRNA pair of SEQ ID NO: 17/SEQ ID NO: 110 or the CRISPR nuclease/gRNA pair of SEQ ID NO:17/SEQ ID NO: 133; the CRISPR nuclease/gRNA pair of SEQ ID NO: 18/SEQ ID NO: 111 or the CRISPR nuclease/gRNA pair of SEQ ID NO: 18/SEQ ID NO: 134; the CRISPR nuclease/gRNA pair of SEQ ID NO: 19/SEQ ID NO: 112 or the CRISPR nuclease/gRNA pair of SEQ ID NO:19/SEQ ID NO: 135; the CRISPR nuclease/gRNA pair of SEQ ID NO:20/SEQ ID NO: 113 or the CRISPR nuclease/gRNA pair of SEQ ID NO:20/SEQ ID NO: 136; the CRISPR nuclease/gRNA pair of SEQ ID NO:21/SEQ ID NO: 114 or the CRISPR nuclease/gRNA pair of SEQ ID NO:21/SEQ ID NO: 137; the CRISPR nuclease/gRNA pair of SEQ ID NO:22/SEQ ID NO: 115 or the CRISPR nuclease/gRNA pair of SEQ ID NO:22/SEQ ID NO: 138; the CRISPR nuclease/gRNA pair of SEQ ID NO:23/SEQ ID NO: 116 or the CRISPR nuclease/gRNA pair of SEQ ID NO:23/SEQ ID NO: 139; the CRISPR nuclease/gRNA pair of SEQ ID NO:24/SEQ ID NO: 140 or the CRISPR nuclease/gRNA pair of SEQ ID NO:24/SEQ ID NO: 164; the CRISPR nuclease/gRNA pair of SEQ ID NO:25/SEQ ID NO: 141 or the CRISPR nuclease/gRNA pair of SEQ ID NO:25/SEQ ID NO: 165; the CRISPR nuclease/gRNA pair of SEQ ID NO:26/SEQ ID NO: 142 or the CRISPR nuclease/gRNA pair of SEQ ID NO:26/SEQ ID NO: 166; the CRISPR nuclease/gRNA pair of SEQ ID NO:27/SEQ ID NO: 143 or the CRISPR nuclease/gRNA pair of SEQ ID NO:27/SEQ ID NO: 167; the CRISPR nuclease/gRNA pair of SEQ ID NO:28/SEQ ID NO: 144 or the CRISPR nuclease/gRNA pair of SEQ ID NO:28/SEQ ID NO: 168; the CRISPR nuclease/gRNA pair of SEQ ID NO:29/SEQ ID NO: 145 or the CRISPR nuclease/gRNA pair of SEQ ID NO:29/SEQ ID NO: 169; the CRISPR nuclease/gRNA pair of SEQ ID NO:30/SEQ ID NO: 146 or the CRISPR nuclease/gRNA pair of SEQ ID NO:30/SEQ ID NO: 170; the CRISPR nuclease/gRNA pair of SEQ ID NO:31/SEQ ID NO: 147 or the CRISPR nuclease/gRNA pair of SEQ ID NO:31/SEQ ID NO: 171; the CRISPR nuclease/gRNA pair of SEQ ID NO:32/SEQ ID NO: 148 or the CRISPR nuclease/gRNA pair of SEQ ID NO:32/SEQ ID NO: 172; the CRISPR nuclease/gRNA pair of SEQ ID NO:33/SEQ ID NO: 149 or the CRISPR nuclease/gRNA pair of SEQ ID NO:33/SEQ ID NO: 173; the CRISPR nuclease/gRNA pair of SEQ ID NO:34/SEQ ID NO: 150 or the CRISPR nuclease/gRNA pair of SEQ ID NO:34/SEQ ID NO: 174; the CRISPR nuclease/gRNA pair of SEQ ID NO:35/SEQ ID NO: 151 or the CRISPR nuclease/gRNA pair of SEQ ID NO:35/SEQ ID NO: 175; the CRISPR nuclease/gRNA pair of SEQ ID NO:36/SEQ ID NO: 152 or the CRISPR nuclease/gRNA pair of SEQ ID NO:36/SEQ ID NO: 176; the CRISPR nuclease/gRNA pair of SEQ ID NO:37/SEQ ID NO: 153 or the CRISPR nuclease/gRNA pair of SEQ ID NO:37/SEQ ID NO: 177; the CRISPR nuclease/gRNA pair of SEQ ID NO:38/SEQ ID NO: 154 or the CRISPR nuclease/gRNA pair of SEQ ID NO:38/SEQ ID NO: 178; the CRISPR nuclease/gRNA pair of SEQ ID NO:39/SEQ ID NO: 155 or the CRISPR nuclease/gRNA pair of SEQ ID NO:39/SEQ ID NO: 179; the CRISPR nuclease/gRNA pair of SEQ ID NO:40/SEQ ID NO: 156 or the CRISPR nuclease/gRNA pair of SEQ ID NO:40/SEQ ID NO: 180; the CRISPR nuclease/gRNA pair of SEQ ID NO:41/SEQ ID NO: 157 or the CRISPR nuclease/gRNA pair of SEQ ID NO:41/SEQ ID NO: 181; the CRISPR nuclease/gRNA pair of SEQ ID NO:42/SEQ ID NO: 158 or the CRISPR nuclease/gRNA pair of SEQ ID NO:42/SEQ ID NO: 182; the CRISPR nuclease/gRNA pair of SEQ ID NO:43/SEQ ID NO: 159 or the CRISPR nuclease/gRNA pair of SEQ ID NO:43/SEQ ID NO: 183; the CRISPR nuclease/gRNA pair of SEQ ID NO:44/SEQ ID NO: 160 or the CRISPR nuclease/gRNA pair of SEQ ID NO:44/SEQ ID NO: 184; the CRISPR nuclease/gRNA pair of SEQ ID NO:45/SEQ ID NO: 161 or the CRISPR nuclease/gRNA pair of SEQ ID NO:45/SEQ ID NO: 185; the CRISPR nuclease/gRNA pair of SEQ ID NO:46/SEQ ID NO: 162 or the CRISPR nuclease/gRNA pair of SEQ ID NO:46/SEQ ID NO: 186; the CRISPR nuclease/gRNA pair of SEQ ID NO:47/SEQ ID NO: 163 or the CRISPR nuclease/gRNA pair of SEQ ID NO:47/SEQ ID NO: 187; the CRISPR nuclease/gRNA pair of SEQ ID NO:48/SEQ ID NO: 188 or the CRISPR nuclease/gRNA pair of SEQ ID NO:48/SEQ ID NO: 234; the CRISPR nuclease/gRNA pair of SEQ ID NO:49/SEQ ID NO: 189 or the CRISPR nuclease/gRNA pair of SEQ ID NO:49/SEQ ID NO: 235; the CRISPR nuclease/gRNA pair of SEQ ID NO:50/SEQ ID NO: 190 or the CRISPR nuclease/gRNA pair of SEQ ID NO:50/SEQ ID NO: 236; the CRISPR nuclease/gRNA pair of SEQ ID NO:51/SEQ ID NO: 191 or the CRISPR nuclease/gRNA pair of SEQ ID NO:51/SEQ ID NO: 237; the CRISPR nuclease/gRNA pair of SEQ ID NO:52/SEQ ID NO: 192 or the CRISPR nuclease/gRNA pair of SEQ ID NO:52/SEQ ID NO: 238; the CRISPR nuclease/gRNA pair of SEQ ID NO:53/SEQ ID NO: 193 or the CRISPR nuclease/gRNA pair of SEQ ID NO:53/SEQ ID NO: 239; the CRISPR nuclease/gRNA pair of SEQ ID NO:54/SEQ ID NO: 194 or the CRISPR nuclease/gRNA pair of SEQ ID NO:54/SEQ ID NO: 240; the CRISPR nuclease/gRNA pair of SEQ ID NO:55/SEQ ID NO: 195 or the CRISPR nuclease/gRNA pair of SEQ ID NO:55/SEQ ID NO: 241; the CRISPR nuclease/gRNA pair of SEQ ID NO:56/SEQ ID NO: 196 or the CRISPR nuclease/gRNA pair of SEQ ID NO:56/SEQ ID NO: 242; the CRISPR nuclease/gRNA pair of SEQ ID NO:57/SEQ ID NO: 197 or the CRISPR nuclease/gRNA pair of SEQ ID NO:57/SEQ ID NO: 243; the CRISPR nuclease/gRNA pair of SEQ ID NO:58/SEQ ID NO: 198 or the CRISPR nuclease/gRNA pair of SEQ ID NO:58/SEQ ID NO: 244; the CRISPR nuclease/gRNA pair of SEQ ID NO:59/SEQ ID NO: 199 or the CRISPR nuclease/gRNA pair of SEQ ID NO:59/SEQ ID NO: 245; the CRISPR nuclease/gRNA pair of SEQ ID NO:60/SEQ ID NO: 200 or the CRISPR nuclease/gRNA pair of SEQ ID NO:60/SEQ ID NO: 246; the CRISPR nuclease/gRNA pair of SEQ ID NO:61/SEQ ID NO: 201 or the CRISPR nuclease/gRNA pair of SEQ ID NO:61/SEQ ID NO: 247; the CRISPR nuclease/gRNA pair of SEQ ID NO:62/SEQ ID NO: 202 or the CRISPR nuclease/gRNA pair of SEQ ID NO:62/SEQ ID NO: 248; the CRISPR nuclease/gRNA pair of SEQ ID NO:63/SEQ ID NO: 203 or the CRISPR nuclease/gRNA pair of SEQ ID NO:63/SEQ ID NO: 249; the CRISPR nuclease/gRNA pair of SEQ ID NO:64/SEQ ID NO: 204 or the CRISPR nuclease/gRNA pair of SEQ ID NO:64/SEQ ID NO: 250; the CRISPR nuclease/gRNA pair of SEQ ID NO:65/SEQ ID NO: 205 or the CRISPR nuclease/gRNA pair of SEQ ID NO:65/SEQ ID NO: 251; the CRISPR nuclease/gRNA pair of SEQ ID NO:66/SEQ ID NO: 206 or the CRISPR nuclease/gRNA pair of SEQ ID NO:66/SEQ ID NO: 252; the CRISPR nuclease/gRNA pair of SEQ ID NO:67/SEQ ID NO: 207 or the CRISPR nuclease/gRNA pair of SEQ ID NO:67/SEQ ID NO: 253; the CRISPR nuclease/gRNA pair of SEQ ID NO:68/SEQ ID NO: 208 or the CRISPR nuclease/gRNA pair of SEQ ID NO:68/SEQ ID NO: 254; the CRISPR nuclease/gRNA pair of SEQ ID NO:69/SEQ ID NO: 209 or the CRISPR nuclease/gRNA pair of SEQ ID NO:69/SEQ ID NO: 255; the CRISPR nuclease/gRNA pair of SEQ ID NO:70/SEQ ID NO: 210 or the CRISPR nuclease/gRNA pair of SEQ ID NO:70/SEQ ID NO: 256; the CRISPR nuclease/gRNA pair of SEQ ID NO:71/SEQ ID NO: 211 or the CRISPR nuclease/gRNA pair of SEQ ID NO:71/SEQ ID NO: 257; the CRISPR nuclease/gRNA pair of SEQ ID NO:72/SEQ ID NO: 212 or the CRISPR nuclease/gRNA pair of SEQ ID NO:72/SEQ ID NO: 258; the CRISPR nuclease/gRNA pair of SEQ ID NO:73/SEQ ID NO: 213 or the CRISPR nuclease/gRNA pair of SEQ ID NO:73/SEQ ID NO: 259; the CRISPR nuclease/gRNA pair of SEQ ID NO:74/SEQ ID NO: 214 or the CRISPR nuclease/gRNA pair of SEQ ID NO:74/SEQ ID NO: 260; the CRISPR nuclease/gRNA pair of SEQ ID NO:75/SEQ ID NO: 215 or the CRISPR nuclease/gRNA pair of SEQ ID NO:75/SEQ ID NO: 261; the CRISPR nuclease/gRNA pair of SEQ ID NO:76/SEQ ID NO: 216 or the CRISPR nuclease/gRNA pair of SEQ ID NO:76/SEQ ID NO: 262; the CRISPR nuclease/gRNA pair of SEQ ID NO:77/SEQ ID NO: 217 or the CRISPR nuclease/gRNA pair of SEQ ID NO:77/SEQ ID NO: 263; the CRISPR nuclease/gRNA pair of SEQ ID NO:78/SEQ ID NO: 218 or the CRISPR nuclease/gRNA pair of SEQ ID NO:78/SEQ ID NO: 264; the CRISPR nuclease/gRNA pair of SEQ ID NO:79/SEQ ID NO: 219 or the CRISPR nuclease/gRNA pair of SEQ ID NO:79/SEQ ID NO: 265; the CRISPR nuclease/gRNA pair of SEQ ID NO:80/SEQ ID NO: 220 or the CRISPR nuclease/gRNA pair of SEQ ID NO:80/SEQ ID NO: 266; the CRISPR nuclease/gRNA pair of SEQ ID NO:81/SEQ ID NO: 221 or the CRISPR nuclease/gRNA pair of SEQ ID NO:81/SEQ ID NO: 267; the CRISPR nuclease/gRNA pair of SEQ ID NO:82/SEQ ID NO: 222 or the CRISPR nuclease/gRNA pair of SEQ ID NO:82/SEQ ID NO: 268; the CRISPR nuclease/gRNA pair of SEQ ID NO:83/SEQ ID NO: 223 or the CRISPR nuclease/gRNA pair of SEQ ID NO:83/SEQ ID NO: 269; the CRISPR nuclease/gRNA pair of SEQ ID NO:84/SEQ ID NO: 224 or the CRISPR nuclease/gRNA pair of SEQ ID NO:84/SEQ ID NO: 270; the CRISPR nuclease/gRNA pair of SEQ ID NO:85/SEQ ID NO: 225 or the CRISPR nuclease/gRNA pair of SEQ ID NO:85/SEQ ID NO: 271; the CRISPR nuclease/gRNA pair of SEQ ID NO:86/SEQ ID NO: 226 or the CRISPR nuclease/gRNA pair of SEQ ID NO:86/SEQ ID NO: 272; the CRISPR nuclease/gRNA pair of SEQ ID NO:87/SEQ ID NO: 227 or the CRISPR nuclease/gRNA pair of SEQ ID NO:87/SEQ ID NO: 273; the CRISPR nuclease/gRNA pair of SEQ ID NO:88/SEQ ID NO: 228 or the CRISPR nuclease/gRNA pair of SEQ ID NO:88/SEQ ID NO: 274; the CRISPR nuclease/gRNA pair of SEQ ID NO:89/SEQ ID NO: 229 or the CRISPR nuclease/gRNA pair of SEQ ID NO:89/SEQ ID NO: 275; the CRISPR nuclease/gRNA pair of SEQ ID NO:90/SEQ ID NO: 230 or the CRISPR nuclease/gRNA pair of SEQ ID NO:90/SEQ ID NO: 276; the CRISPR nuclease/gRNA pair of SEQ ID NO:91/SEQ ID NO: 231 or the CRISPR nuclease/gRNA pair of SEQ ID NO:91/SEQ ID NO: 277; the CRISPR nuclease/gRNA pair of SEQ ID NO:92/SEQ ID NO: 232 or the CRISPR nuclease/gRNA pair of SEQ ID NO:92/SEQ ID NO: 278; the CRISPR nuclease/gRNA pair of SEQ ID NO:93/SEQ ID NO: 233 and/or the CRISPR nuclease/gRNA pair of SEQ ID NO:93/SEQ ID NO: 279; the CRISPR nuclease/gRNA pair is SEQ ID NO:22/SEQ ID NO: 115 or the CRISPR nuclease/gRNA pair of SEQ ID NO:22/SEQ ID NO: 138.

Embodiment 6. The reaction mixture of any one of Embodiments 1-5, wherein at least one nucleic acid-guided CRISPR nuclease has trans-cleavage activity.

Embodiment 7. The reaction mixture of any one of Embodiments 1-6, wherein at least one nucleic acid-guided CRISPR nuclease hybridizes to a PAM region in a target nucleic acid sequence.

Embodiment 8. The reaction mixture of any one of Embodiments 1-7, wherein the substrate is a ribonucleic acid molecule (RNA) complexed with a fluorophore and a quencher for the fluorophore.

Embodiment 9. The reaction mixture of any one of Embodiments 1-8, wherein the reaction mixture is in solution, and wherein the substrate is present in the reaction mix in a concentration ranging from 250 nM to 450 nM.

Embodiment 10. The reaction mixture of any one of Embodiments 1-9, wherein the substrate is a ribonucleic acid molecule (RNA) complexed with biotin.

Embodiment 11. The reaction mixture of any one of Embodiments 1-9, wherein the substrate is a deoxyribonucleic acid (DNA) complexed with a fluorophore and a quencher for the fluorophore.

Embodiment 12. The reaction mixture of any one of Embodiments 1-11, wherein the reaction mixture further comprises a target nucleic acid sequence.

Embodiment 13. The reaction mixture of any one of Embodiments 1-12, wherein the target nucleic acid sequence is a sequence from an influenza A virus, an influenza B virus, a Respiratory Syncytial Virus (RSV), or a SARS-CoV-2 virus.

Embodiment 14. The reaction mixture of any one of Embodiments 1-13, wherein the target nucleic acid sequence is from an influenza A virus.

Embodiment 15. The reaction mixture of any one of Embodiments 1-14, wherein the target nucleic acid sequence is SEQ ID NO:373.

Embodiment 16. The reaction mixture of any one of Embodiments 1-15, wherein the target nucleic acid sequence activates cleavage of the substrate by the nucleic acid-guided CRISPR nuclease.

Embodiment 17. The reaction mixture of any one of Embodiments 1-16, wherein the reaction mixture is in solution, and wherein the nucleic acid-guided CRISPR nuclease cleaves the substrate with a sensitivity of at least 25 nM.

Embodiment 18. The reaction mixture of any one of Embodiments 1-17, wherein the nucleic acid-guided CRISPR nuclease is present in the reaction mix in a concentration ranging from 10 ng/uL to 100 ng/uL.

Embodiment 19. The reaction mixture of any one of Embodiments 1-18, wherein the guide nucleic acid (gRNA) is present in the reaction mix in a concentration ranging from 20 nM to 180 nM.

Embodiment 20. The reaction mixture of any one of Embodiments 1-19, wherein the guide nucleic acid (gRNA) is from 40 to 65 nucleotides in length.

Embodiment 21. The reaction mixture of any one of Embodiments 1-20, wherein the reaction mixture further comprises an RNase inhibitor and MgCl₂.

Embodiment 22. The reaction mixture of any one of Embodiments 1-21, wherein the reaction mixture further comprises another gRNA.

Embodiment 23. The reaction mixture of any one of Embodiments 1-22, wherein the complex comprises the CRISPR nuclease/direct repeat pair of SEQ ID NO:22/SEQ ID NO: 395, the CRISPR nuclease/direct repeat pair of SEQ ID NO:62/SEQ ID NO: 435, the CRISPR nuclease/direct repeat pair of SEQ ID NO:80/SEQ ID NO: 453, and/or CRISPR nuclease/direct repeat pair of SEQ ID NO:91/SEQ ID NO: 464.

Embodiment 24. An expression vector encoding a nucleic acid-guided CRISPR nuclease having at least 85% amino acid homology to any one of SEQ ID NOs:1-93, wherein the expression vector includes a heterologous promoter.

Embodiment 25. The expression vector of Embodiment 24, wherein the heterologous promoter is a T7 promoter.

Embodiment 26. The expression vector of any one of Embodiments 24 or 25, wherein the expression vector further comprises a purification tag.

Embodiment 27. The expression vector of any one of Embodiments 24-26, wherein the purification tag is a 6-his tag.

Embodiment 28. The expression vector of Embodiment 27, wherein the purification tag is on an N-terminus of the nucleic acid-guided CRISPR nuclease.

Embodiment 29. The expression vector of any one of Embodiments 24-28, wherein the purification tag is on a C-terminus of the nucleic acid-guided CRISPR nuclease.

Embodiment 30. The expression vector of any one of Embodiments 24-29, wherein the purification tag is cleavable.

Embodiment 31. The expression vector of any one of Embodiments 24-30, wherein the nucleic acid-guided CRISPR nuclease has at least 90% amino acid homology to any one of SEQ ID NOs:1-93.

Embodiment 32. The expression vector of any one of Embodiments 24-31, wherein the nucleic acid-guided CRISPR nuclease has at least 95% amino acid homology to any one of SEQ ID NOs:1-93.

Embodiment 33. The expression vector of any one of Embodiments 24-32, wherein the nucleic acid-guided CRISPR nuclease has at least 99% amino acid homology to any one of SEQ ID NOs:1-93.

Embodiment 34. The expression vector of any one of Embodiments 24-33, wherein the nucleic acid-guided CRISPR nuclease is selected from the group consisting of SEQ ID NO:22, SEQ ID NO:62, SEQ ID NO:80, and SEQ ID NO:91.

Embodiment 35. A recombinant nucleic acid-guided CRISPR nuclease produced from a cell-free expression system from an expression vector of any one of Embodiments 24-34.

Embodiment 36. The recombinant nucleic acid-guided CRISPR nuclease of Embodiment 35, wherein the recombinant nucleic acid-guided CRISPR nuclease has trans-cleavage activity.

Embodiment 37. The recombinant nucleic acid-guided CRISPR nuclease of Embodiment 36, wherein the recombinant nucleic acid-guided CRISPR nuclease cleaves a single stranded deoxyribonucleic acid (DNA) molecule.

Embodiment 38. The recombinant nucleic acid-guided CRISPR nuclease of Embodiment 36, wherein the recombinant nucleic acid-guided CRISPR nuclease cleaves a double stranded deoxyribonucleic acid (DNA) molecule.

Embodiment 39. The recombinant nucleic acid-guided CRISPR nuclease of any one of Embodiments 36-38, wherein the recombinant nucleic acid-guided CRISPR nuclease cleaves a ribonucleic acid (RNA) molecule.

Embodiment 40. The recombinant nucleic acid-guided CRISPR nuclease of any one of Embodiments 36-38, wherein the recombinant nucleic acid-guided CRISPR nuclease exhibits at least 25% higher cleavage activity as compared to LwaCas13a when cleaving SEQ ID NO: 180.

Embodiment 41. The recombinant nucleic acid-guided CRISPR nuclease of any one of Embodiments 36-40, the recombinant nucleic acid-guided CRISPR nuclease comprises SEQ ID NO:22, SEQ ID NO:62, or SEQ ID NO:80.

Embodiment 42. The recombinant nucleic acid-guided CRISPR nuclease of any one of Embodiments 36-41, wherein the recombinant nucleic acid-guided CRISPR nuclease has an apparent weight of 90 kDa to 170 kDa when produced from an in-vitro translation system.

Embodiment 43. A nucleic acid sequence having a first portion at least 95% identical to any one of SEQ ID NOs:280-372, wherein the nucleic acid sequence encodes a nucleic acid-guided CRISPR nuclease comprising an amino acid sequence of any one of SEQ ID NOs:1-93 or comprising an amino acid sequence at least 95% identical to any one of SEQ ID Nos:1-93 and, optionally, at least one nucleic acid sequence configured to facilitate the recombinant expression of the nucleic acid-guided CRISPR nuclease in an organism.

Embodiment 44. The nucleic acid of Embodiment 43, wherein the nucleic acid-guided CRISPR nuclease is recombinantly expressed in an organism.

Embodiment 45. A nucleic acid sequence, wherein the nucleic acid sequence comprises a nucleic acid of any one of Embodiments 43 and 44, that has been codon optimized for expression in E. coli.

Embodiment 46. An enzyme system comprising: a nucleic acid-guided CRISPR nuclease having at least 85% amino acid homology to any one of SEQ ID NOs:1-93; and a guide nucleic acid (gRNA) for complexing with the nucleic acid-guided CRISPR nuclease, wherein the gRNA comprises a nucleic acid sequence, e.g., a gRNA sequence, wherein the nucleic acid sequence comprises (i) a direct repeat sequence having at least 68%, at least 85%, at least 90%, or at least 95% homology to any one of SEQ ID Nos: 374-466, and optionally, (ii) at least 85% homology to any one of SEQ ID NOs:94-279.

Embodiment 47. An enzyme system comprising: a nucleic acid-guided CRISPR nuclease having at least 85% amino acid homology to any one of SEQ ID NOs:1-93; and a guide nucleic acid (gRNA) for complexing with the nucleic acid-guided CRISPR nuclease, wherein the gRNA comprises a nucleic acid sequence, e.g., a gRNA sequence, wherein the nucleic acid sequence comprises (i) a direct repeat sequence having at least 65%, at least 85%, at least 90%, or at least 95% homology to any one of SEQ ID Nos: 374-466, and optionally (ii) at least 65%, at least 85%, at least 90%, or at least 95% nucleic acid homology to any one of SEQ ID NOs:94-279.

Embodiment 48. The enzyme system of any one of Embodiment 46 or Embodiment 47, wherein the nucleic acid-guided CRISPR nuclease has at least 90% amino acid homology to any one of SEQ ID NOs:1-93.

Embodiment 49. The enzyme system of any one of Embodiment 46-48, whereby the nucleic acid-guided CRISPR nuclease has at least 95% or at least 99% amino acid homology to any one of SEQ ID NOs:1-93.

Embodiment 50. The enzyme system of any one of Embodiment 46-49, whereby the nucleic acid system comprises a CRISPR nuclease that complexes with a guide nucleic acid comprising a direct repeat sequence, thereby forming one or more CRISPR nuclease/direct repeat pair complexes selected from the CRISPR nuclease/direct repeat pairs of SEQ ID NO:1/SEQ ID NO: 374 (JW13101); the CRISPR nuclease/direct repeat pair of SEQ ID NO:2/SEQ ID NO: 375 (JW13102); the CRISPR nuclease/direct repeat pair of SEQ ID NO:3/SEQ ID NO: 376 (JW13103); the CRISPR nuclease/direct repeat pair of SEQ ID NO:4/SEQ ID NO: 377 (JW13104); the CRISPR nuclease/direct repeat pair of SEQ ID NO:5/SEQ ID NO: 378 (JW13105); the CRISPR nuclease/direct repeat pair of SEQ ID NO:6/SEQ ID NO: 379 (JW13106); the CRISPR nuclease/direct repeat pair of SEQ ID NO:7/SEQ ID NO: 380 (JW13107); the CRISPR nuclease/direct repeat pair of SEQ ID NO:8/SEQ ID NO: 381 (JW13108); the CRISPR nuclease/direct repeat pair of SEQ ID NO:9/SEQ ID NO: 382 (JW13110); the CRISPR nuclease/direct repeat pair of SEQ ID NO: 10/SEQ ID NO: 383 (JW13112); the CRISPR nuclease/direct repeat pair of SEQ ID NO: 11/SEQ ID NO: 384 (JW13113); the CRISPR nuclease/direct repeat pair of SEQ ID NO: 12/SEQ ID NO: 385 (JW13114); the CRISPR nuclease/direct repeat pair of SEQ ID NO: 13/SEQ ID NO: 386 (JW13115); the CRISPR nuclease/direct repeat pair of SEQ ID NO: 14/SEQ ID NO: 387 (JW13116); the CRISPR nuclease/direct repeat pair of SEQ ID NO: 15/SEQ ID NO: 388 (JW13117); the CRISPR nuclease/direct repeat pair of SEQ ID NO:16/SEQ ID NO: 389 (JW13118); the CRISPR nuclease/direct repeat pair of SEQ ID NO: 17/SEQ ID NO: 390 (JW13119); the CRISPR nuclease/direct repeat pair of SEQ ID NO: 18/SEQ ID NO: 391 (JW13120); the CRISPR nuclease/direct repeat pair of SEQ ID NO: 19/SEQ ID NO: 392 (JW13121); the CRISPR nuclease/direct repeat pair of SEQ ID NO:20/SEQ ID NO: 393 (JW13122); the CRISPR nuclease/direct repeat pair of SEQ ID NO:21/SEQ ID NO: 394 (JW13123); the CRISPR nuclease/direct repeat pair of SEQ ID NO:22/SEQ ID NO: 395 (JW13124); the CRISPR nuclease/direct repeat pair of SEQ ID NO:23/SEQ ID NO: 396 (JW13125); the CRISPR nuclease/direct repeat pair of SEQ ID NO:24/SEQ ID NO: 397 (JW13126); the CRISPR nuclease/direct repeat pair of SEQ ID NO:25/SEQ ID NO: 398 (JW13127); the CRISPR nuclease/direct repeat pair of SEQ ID NO:26/SEQ ID NO: 399 (JW13128); the CRISPR nuclease/direct repeat pair of SEQ ID NO:27/SEQ ID NO: 400 (JW13129); the CRISPR nuclease/direct repeat pair of SEQ ID NO:28/SEQ ID NO: 401 (JW13130); the CRISPR nuclease/direct repeat pair of SEQ ID NO:29/SEQ ID NO: 402 (JW13131); the CRISPR nuclease/direct repeat pair of SEQ ID NO:30/SEQ ID NO: 403 (JW13132); the CRISPR nuclease/direct repeat pair of SEQ ID NO:31/SEQ ID NO: 404 (JW13133); the CRISPR nuclease/direct repeat pair of SEQ ID NO:32/SEQ ID NO: 405 (JW13134); the CRISPR nuclease/direct repeat pair of SEQ ID NO:33/SEQ ID NO: 406 (JW13135); the CRISPR nuclease/direct repeat pair of SEQ ID NO:34/SEQ ID NO: 407 (JW13136); the CRISPR nuclease/direct repeat pair of SEQ ID NO:35/SEQ ID NO: 408 (JW13137); the CRISPR nuclease/direct repeat pair of SEQ ID NO:36/SEQ ID NO: 409 (JW13138); the CRISPR nuclease/direct repeat pair of SEQ ID NO:37/SEQ ID NO: 410 (JW13139); the CRISPR nuclease/direct repeat pair of SEQ ID NO:38/SEQ ID NO: 411 (JW13140); the CRISPR nuclease/direct repeat pair of SEQ ID NO:39/SEQ ID NO: 412 (JW13141); the CRISPR nuclease/direct repeat pair of SEQ ID NO:40/SEQ ID NO: 413 (JW13142); the CRISPR nuclease/direct repeat pair of SEQ ID NO:41/SEQ ID NO: 414 (JW13143); the CRISPR nuclease/direct repeat pair of SEQ ID NO:42/SEQ ID NO: 415 (JW13144); the CRISPR nuclease/direct repeat pair of SEQ ID NO:43/SEQ ID NO: 416 (JW13145); the CRISPR nuclease/direct repeat pair of SEQ ID NO:44/SEQ ID NO: 417 (JW13146); the CRISPR nuclease/direct repeat pair of SEQ ID NO:45/SEQ ID NO: 418 (JW13147); the CRISPR nuclease/direct repeat pair of SEQ ID NO:46/SEQ ID NO: 419 (JW13148); the CRISPR nuclease/direct repeat pair of SEQ ID NO:47/SEQ ID NO: 420 (JW13149); the CRISPR nuclease/direct repeat pair of SEQ ID NO:48/SEQ ID NO: 421 (JW13150); the CRISPR nuclease/direct repeat pair of SEQ ID NO:49/SEQ ID NO: 422 (JW13153); the CRISPR nuclease/direct repeat pair of SEQ ID NO:50/SEQ ID NO: 423 (JW13154); the CRISPR nuclease/direct repeat pair of SEQ ID NO:51/SEQ ID NO: 424 (JW13155); the CRISPR nuclease/direct repeat pair of SEQ ID NO:52/SEQ ID NO: 425 (JW13156); the CRISPR nuclease/direct repeat pair of SEQ ID NO:53/SEQ ID NO: 426 (JW13157); the CRISPR nuclease/direct repeat pair of SEQ ID NO:54/SEQ ID NO: 427 (JW13158); the CRISPR nuclease/direct repeat pair of SEQ ID NO:55/SEQ ID NO: 428 (JW13159); the CRISPR nuclease/direct repeat pair of SEQ ID NO:56/SEQ ID NO: 429 (JW13160); the CRISPR nuclease/direct repeat pair of SEQ ID NO:57/SEQ ID NO: 430 (JW13161); the CRISPR nuclease/direct repeat pair of SEQ ID NO:58/SEQ ID NO: 431 (JW13162); the CRISPR nuclease/direct repeat pair of SEQ ID NO:59/SEQ ID NO: 432 (JW13163); the CRISPR nuclease/direct repeat pair of SEQ ID NO: 60/SEQ ID NO: 433 (JW13164); the CRISPR nuclease/direct repeat pair of SEQ ID NO:61/SEQ ID NO: 434 (JW13165); the CRISPR nuclease/direct repeat pair of SEQ ID NO:62/SEQ ID NO: 435 (JW13166); the CRISPR nuclease/direct repeat pair of SEQ ID NO:63/SEQ ID NO: 436 (JW13167); the CRISPR nuclease/direct repeat pair of SEQ ID NO:64/SEQ ID NO: 437 (JW13168); the CRISPR nuclease/direct repeat pair of SEQ ID NO:65/SEQ ID NO: 438 (JW13169); the CRISPR nuclease/direct repeat pair of SEQ ID NO:66/SEQ ID NO: 439 (JW13170); the CRISPR nuclease/direct repeat pair of SEQ ID NO:67/SEQ ID NO: 440 (JW13171); the CRISPR nuclease/direct repeat pair of SEQ ID NO:68/SEQ ID NO: 441 (JW13172); the CRISPR nuclease/direct repeat pair of SEQ ID NO:69/SEQ ID NO: 442 (JW13173); the CRISPR nuclease/direct repeat pair of SEQ ID NO:70/SEQ ID NO: 443 (JW13174); the CRISPR nuclease/direct repeat pair of SEQ ID NO:71/SEQ ID NO: 444 (JW13175); the CRISPR nuclease/direct repeat pair of SEQ ID NO:72/SEQ ID NO: 445 (JW13176); the CRISPR nuclease/direct repeat pair of SEQ ID NO:73/SEQ ID NO: 446 (JW13177); the CRISPR nuclease/direct repeat pair of SEQ ID NO:74/SEQ ID NO: 447 (JW13178); the CRISPR nuclease/direct repeat pair of SEQ ID NO:75/SEQ ID NO: 448 (JW13179); the CRISPR nuclease/direct repeat pair of SEQ ID NO: 76/SEQ ID NO: 449 (JW13180); the CRISPR nuclease/direct repeat pair of SEQ ID NO:77/SEQ ID NO: 450 (JW13181); the CRISPR nuclease/direct repeat pair of SEQ ID NO:78/SEQ ID NO: 451 (JW13182); the CRISPR nuclease/direct repeat pair of SEQ ID NO:79/SEQ ID NO: 452 (JW13183); the CRISPR nuclease/direct repeat pair of SEQ ID NO:80/SEQ ID NO: 453 (JW13184); the CRISPR nuclease/direct repeat pair of SEQ ID NO:81/SEQ ID NO: 454 (JW13185); the CRISPR nuclease/direct repeat pair of SEQ ID NO:82/SEQ ID NO: 455 (JW13186); the CRISPR nuclease/direct repeat pair of SEQ ID NO:83/SEQ ID NO: 456 (JW13187); the CRISPR nuclease/direct repeat pair of SEQ ID NO:84/SEQ ID NO: 457 (JW13188); the CRISPR nuclease/direct repeat pair of SEQ ID NO:85/SEQ ID NO: 458 (JW13189); the CRISPR nuclease/direct repeat pair of SEQ ID NO:86/SEQ ID NO: 459 (JW13190); the CRISPR nuclease/direct repeat pair of SEQ ID NO:87/SEQ ID NO: 460 (JW13191); the CRISPR nuclease/direct repeat pair of SEQ ID NO:88/SEQ ID NO: 461 (JW13192); the CRISPR nuclease/direct repeat pair of SEQ ID NO:89/SEQ ID NO: 462 (JW13193); the CRISPR nuclease/direct repeat pair of SEQ ID NO:90/SEQ ID NO: 463 (JW13194); the CRISPR nuclease/direct repeat pair of SEQ ID NO:91/SEQ ID NO: 464 (JW13195); the CRISPR nuclease/direct repeat pair of SEQ ID NO:92/SEQ ID NO: 465 (JW13196); and/or the CRISPR nuclease/direct repeat pair of SEQ ID NO:93/SEQ ID NO: 466 (JW13197).

Embodiment 51. The enzyme system of any one of Embodiment 46-50, whereby the nucleic acid-guided CRISPR nuclease and the guide nucleic acid comprise a CRISPR nuclease/gRNA pair selected from the group consisting of: the CRISPR nuclease/gRNA pair of SEQ ID NO:1/SEQ ID NO: 94 or the CRISPR nuclease/gRNA pair of SEQ ID NO:1/SEQ ID NO: 117; the CRISPR nuclease/gRNA pair of SEQ ID NO:2/SEQ ID NO: 95 or the CRISPR nuclease/gRNA pair of SEQ ID NO:2/SEQ ID NO: 118; the CRISPR nuclease/gRNA pair of SEQ ID NO:3/SEQ ID NO: 96 or the CRISPR nuclease/gRNA pair of SEQ ID NO:3/SEQ ID NO: 119; the CRISPR nuclease/gRNA pair of SEQ ID NO:4/SEQ ID NO: 97 or the CRISPR nuclease/gRNA pair of SEQ ID NO:4/SEQ ID NO: 120; the CRISPR nuclease/gRNA pair of SEQ ID NO:5/SEQ ID NO: 98 or the CRISPR nuclease/gRNA pair of SEQ ID NO:5/SEQ ID NO: 121; the CRISPR nuclease/gRNA pair of SEQ ID NO:6/SEQ ID NO: 99 or the CRISPR nuclease/gRNA pair of SEQ ID NO:6/SEQ ID NO: 122; the CRISPR nuclease/gRNA pair of SEQ ID NO:7/SEQ ID NO: 100 or the CRISPR nuclease/gRNA pair of SEQ ID NO:7/SEQ ID NO: 123; the CRISPR nuclease/gRNA pair of SEQ ID NO:8/SEQ ID NO: 101 or the CRISPR nuclease/gRNA pair of SEQ ID NO:8/SEQ ID NO: 124; the CRISPR nuclease/gRNA pair of SEQ ID NO:9/SEQ ID NO: 102 or the CRISPR nuclease/gRNA pair of SEQ ID NO:9/SEQ ID NO: 125; the CRISPR nuclease/gRNA pair of SEQ ID NO: 10/SEQ ID NO: 103 or the CRISPR nuclease/gRNA pair of SEQ ID NO: 10/SEQ ID NO: 126; the CRISPR nuclease/gRNA pair of SEQ ID NO: 11/SEQ ID NO: 104 or the CRISPR nuclease/gRNA pair of SEQ ID NO:11/SEQ ID NO: 127; the CRISPR nuclease/gRNA pair of SEQ ID NO: 12/SEQ ID NO: 105 or the CRISPR nuclease/gRNA pair of SEQ ID NO: 12/SEQ ID NO: 128; the CRISPR nuclease/gRNA pair of SEQ ID NO: 13/SEQ ID NO: 106 or the CRISPR nuclease/gRNA pair of SEQ ID NO:13/SEQ ID NO: 129; the CRISPR nuclease/gRNA pair of SEQ ID NO:14/SEQ ID NO: 107 or the CRISPR nuclease/gRNA pair of SEQ ID NO: 14/SEQ ID NO: 130; the CRISPR nuclease/gRNA pair of SEQ ID NO: 15/SEQ ID NO: 108 or the CRISPR nuclease/gRNA pair of SEQ ID NO: 15/SEQ ID NO: 131; the CRISPR nuclease/gRNA pair of SEQ ID NO: 16/SEQ ID NO: 109 or the CRISPR nuclease/gRNA pair of SEQ ID NO: 16/SEQ ID NO: 132; the CRISPR nuclease/gRNA pair of SEQ ID NO: 17/SEQ ID NO: 110 or the CRISPR nuclease/gRNA pair of SEQ ID NO: 17/SEQ ID NO: 133; the CRISPR nuclease/gRNA pair of SEQ ID NO:18/SEQ ID NO: 111 or the CRISPR nuclease/gRNA pair of SEQ ID NO:18/SEQ ID NO: 134; the CRISPR nuclease/gRNA pair of SEQ ID NO: 19/SEQ ID NO: 112 or the CRISPR nuclease/gRNA pair of SEQ ID NO: 19/SEQ ID NO: 135; the CRISPR nuclease/gRNA pair of SEQ ID NO:20/SEQ ID NO: 113 or the CRISPR nuclease/gRNA pair of SEQ ID NO:20/SEQ ID NO: 136; the CRISPR nuclease/gRNA pair of SEQ ID NO:21/SEQ ID NO: 114 or the CRISPR nuclease/gRNA pair of SEQ ID NO:21/SEQ ID NO: 137; the CRISPR nuclease/gRNA pair of SEQ ID NO:22/SEQ ID NO: 115 or the CRISPR nuclease/gRNA pair of SEQ ID NO:22/SEQ ID NO: 138; the CRISPR nuclease/gRNA pair of SEQ ID NO:23/SEQ ID NO: 116 or the CRISPR nuclease/gRNA pair of SEQ ID NO:23/SEQ ID NO: 139; the CRISPR nuclease/gRNA pair of SEQ ID NO:24/SEQ ID NO: 140 or the CRISPR nuclease/gRNA pair of SEQ ID NO:24/SEQ ID NO: 164; the CRISPR nuclease/gRNA pair of SEQ ID NO:25/SEQ ID NO: 141 or the CRISPR nuclease/gRNA pair of SEQ ID NO:25/SEQ ID NO: 165; the CRISPR nuclease/gRNA pair of SEQ ID NO:26/SEQ ID NO: 142 or the CRISPR nuclease/gRNA pair of SEQ ID NO:26/SEQ ID NO: 166; the CRISPR nuclease/gRNA pair of SEQ ID NO:27/SEQ ID NO: 143 or the CRISPR nuclease/gRNA pair of SEQ ID NO:27/SEQ ID NO: 167; the CRISPR nuclease/gRNA pair of SEQ ID NO:28/SEQ ID NO: 144 or the CRISPR nuclease/gRNA pair of SEQ ID NO:28/SEQ ID NO: 168; the CRISPR nuclease/gRNA pair of SEQ ID NO:29/SEQ ID NO: 145 or the CRISPR nuclease/gRNA pair of SEQ ID NO:29/SEQ ID NO: 169; the CRISPR nuclease/gRNA pair of SEQ ID NO:30/SEQ ID NO: 146 or the CRISPR nuclease/gRNA pair of SEQ ID NO:30/SEQ ID NO: 170; the CRISPR nuclease/gRNA pair of SEQ ID NO:31/SEQ ID NO: 147 or the CRISPR nuclease/gRNA pair of SEQ ID NO:31/SEQ ID NO: 171; the CRISPR nuclease/gRNA pair of SEQ ID NO:32/SEQ ID NO: 148 or the CRISPR nuclease/gRNA pair of SEQ ID NO:32/SEQ ID NO: 172; the CRISPR nuclease/gRNA pair of SEQ ID NO:33/SEQ ID NO: 149 or the CRISPR nuclease/gRNA pair of SEQ ID NO:33/SEQ ID NO: 173; the CRISPR nuclease/gRNA pair of SEQ ID NO:34/SEQ ID NO: 150 or the CRISPR nuclease/gRNA pair of SEQ ID NO:34/SEQ ID NO: 174; the CRISPR nuclease/gRNA pair of SEQ ID NO:35/SEQ ID NO: 151 or the CRISPR nuclease/gRNA pair of SEQ ID NO:35/SEQ ID NO: 175; the CRISPR nuclease/gRNA pair of SEQ ID NO:36/SEQ ID NO: 152 or the CRISPR nuclease/gRNA pair of SEQ ID NO:36/SEQ ID NO: 176; the CRISPR nuclease/gRNA pair of SEQ ID NO:37/SEQ ID NO: 153 or the CRISPR nuclease/gRNA pair of SEQ ID NO:37/SEQ ID NO: 177; the CRISPR nuclease/gRNA pair of SEQ ID NO:38/SEQ ID NO: 154 or the CRISPR nuclease/gRNA pair of SEQ ID NO:38/SEQ ID NO: 178; the CRISPR nuclease/gRNA pair of SEQ ID NO:39/SEQ ID NO: 155 or the CRISPR nuclease/gRNA pair of SEQ ID NO:39/SEQ ID NO: 179; the CRISPR nuclease/gRNA pair of SEQ ID NO:40/SEQ ID NO: 156 or the CRISPR nuclease/gRNA pair of SEQ ID NO:40/SEQ ID NO: 180; the CRISPR nuclease/gRNA pair of SEQ ID NO:41/SEQ ID NO: 157 or the CRISPR nuclease/gRNA pair of SEQ ID NO:41/SEQ ID NO: 181; the CRISPR nuclease/gRNA pair of SEQ ID NO:42/SEQ ID NO: 158 or the CRISPR nuclease/gRNA pair of SEQ ID NO:42/SEQ ID NO: 182; the CRISPR nuclease/gRNA pair of SEQ ID NO:43/SEQ ID NO: 159 or the CRISPR nuclease/gRNA pair of SEQ ID NO:43/SEQ ID NO: 183; the CRISPR nuclease/gRNA pair of SEQ ID NO:44/SEQ ID NO: 160 or the CRISPR nuclease/gRNA pair of SEQ ID NO:44/SEQ ID NO: 184; the CRISPR nuclease/gRNA pair of SEQ ID NO:45/SEQ ID NO: 161 or the CRISPR nuclease/gRNA pair of SEQ ID NO:45/SEQ ID NO: 185; the CRISPR nuclease/gRNA pair of SEQ ID NO:46/SEQ ID NO: 162 or the CRISPR nuclease/gRNA pair of SEQ ID NO:46/SEQ ID NO: 186; the CRISPR nuclease/gRNA pair of SEQ ID NO:47/SEQ ID NO: 163 or the CRISPR nuclease/gRNA pair of SEQ ID NO:47/SEQ ID NO: 187; the CRISPR nuclease/gRNA pair of SEQ ID NO:48/SEQ ID NO: 188 or the CRISPR nuclease/gRNA pair of SEQ ID NO:48/SEQ ID NO: 234; the CRISPR nuclease/gRNA pair of SEQ ID NO:49/SEQ ID NO: 189 or the CRISPR nuclease/gRNA pair of SEQ ID NO:49/SEQ ID NO: 235; the CRISPR nuclease/gRNA pair of SEQ ID NO:50/SEQ ID NO: 190 or the CRISPR nuclease/gRNA pair of SEQ ID NO:50/SEQ ID NO: 236; the CRISPR nuclease/gRNA pair of SEQ ID NO:51/SEQ ID NO: 191 or the CRISPR nuclease/gRNA pair of SEQ ID NO:51/SEQ ID NO: 237; the CRISPR nuclease/gRNA pair of SEQ ID NO:52/SEQ ID NO: 192 or the CRISPR nuclease/gRNA pair of SEQ ID NO:52/SEQ ID NO: 238; the CRISPR nuclease/gRNA pair of SEQ ID NO:53/SEQ ID NO: 193 or the CRISPR nuclease/gRNA pair of SEQ ID NO:53/SEQ ID NO: 239; the CRISPR nuclease/gRNA pair of SEQ ID NO:54/SEQ ID NO: 194 or the CRISPR nuclease/gRNA pair of SEQ ID NO:54/SEQ ID NO: 240; the CRISPR nuclease/gRNA pair of SEQ ID NO:55/SEQ ID NO: 195 or the CRISPR nuclease/gRNA pair of SEQ ID NO:55/SEQ ID NO: 241; the CRISPR nuclease/gRNA pair of SEQ ID NO:56/SEQ ID NO: 196 or the CRISPR nuclease/gRNA pair of SEQ ID NO:56/SEQ ID NO: 242; the CRISPR nuclease/gRNA pair of SEQ ID NO:57/SEQ ID NO: 197 or the CRISPR nuclease/gRNA pair of SEQ ID NO:57/SEQ ID NO: 243; the CRISPR nuclease/gRNA pair of SEQ ID NO:58/SEQ ID NO: 198 or the CRISPR nuclease/gRNA pair of SEQ ID NO:58/SEQ ID NO: 244; the CRISPR nuclease/gRNA pair of SEQ ID NO:59/SEQ ID NO: 199 or the CRISPR nuclease/gRNA pair of SEQ ID NO:59/SEQ ID NO: 245; the CRISPR nuclease/gRNA pair of SEQ ID NO:60/SEQ ID NO: 200 or the CRISPR nuclease/gRNA pair of SEQ ID NO:60/SEQ ID NO: 246; the CRISPR nuclease/gRNA pair of SEQ ID NO:61/SEQ ID NO: 201 or the CRISPR nuclease/gRNA pair of SEQ ID NO:61/SEQ ID NO: 247; the CRISPR nuclease/gRNA pair of SEQ ID NO:62/SEQ ID NO: 202 or the CRISPR nuclease/gRNA pair of SEQ ID NO:62/SEQ ID NO: 248; the CRISPR nuclease/gRNA pair of SEQ ID NO:63/SEQ ID NO: 203 or the CRISPR nuclease/gRNA pair of SEQ ID NO:63/SEQ ID NO: 249; the CRISPR nuclease/gRNA pair of SEQ ID NO:64/SEQ ID NO: 204 or the CRISPR nuclease/gRNA pair of SEQ ID NO:64/SEQ ID NO: 250; the CRISPR nuclease/gRNA pair of SEQ ID NO:65/SEQ ID NO: 205 or the CRISPR nuclease/gRNA pair of SEQ ID NO:65/SEQ ID NO: 251; the CRISPR nuclease/gRNA pair of SEQ ID NO:66/SEQ ID NO: 206 or the CRISPR nuclease/gRNA pair of SEQ ID NO:66/SEQ ID NO: 252; the CRISPR nuclease/gRNA pair of SEQ ID NO:67/SEQ ID NO: 207 or the CRISPR nuclease/gRNA pair of SEQ ID NO:67/SEQ ID NO: 253; the CRISPR nuclease/gRNA pair of SEQ ID NO:68/SEQ ID NO: 208 or the CRISPR nuclease/gRNA pair of SEQ ID NO:68/SEQ ID NO: 254; the CRISPR nuclease/gRNA pair of SEQ ID NO:69/SEQ ID NO: 209 or the CRISPR nuclease/gRNA pair of SEQ ID NO:69/SEQ ID NO: 255; the CRISPR nuclease/gRNA pair of SEQ ID NO:70/SEQ ID NO: 210 or the CRISPR nuclease/gRNA pair of SEQ ID NO:70/SEQ ID NO: 256; the CRISPR nuclease/gRNA pair of SEQ ID NO:71/SEQ ID NO: 211 or the CRISPR nuclease/gRNA pair of SEQ ID NO:71/SEQ ID NO: 257; the CRISPR nuclease/gRNA pair of SEQ ID NO:72/SEQ ID NO: 212 or the CRISPR nuclease/gRNA pair of SEQ ID NO:72/SEQ ID NO: 258; the CRISPR nuclease/gRNA pair of SEQ ID NO:73/SEQ ID NO: 213 or the CRISPR nuclease/gRNA pair of SEQ ID NO:73/SEQ ID NO: 259; the CRISPR nuclease/gRNA pair of SEQ ID NO:74/SEQ ID NO: 214 or the CRISPR nuclease/gRNA pair of SEQ ID NO:74/SEQ ID NO: 260; the CRISPR nuclease/gRNA pair of SEQ ID NO:75/SEQ ID NO: 215 or the CRISPR nuclease/gRNA pair of SEQ ID NO:75/SEQ ID NO: 261; the CRISPR nuclease/gRNA pair of SEQ ID NO:76/SEQ ID NO: 216 or the CRISPR nuclease/gRNA pair of SEQ ID NO:76/SEQ ID NO: 262; the CRISPR nuclease/gRNA pair of SEQ ID NO:77/SEQ ID NO: 217 or the CRISPR nuclease/gRNA pair of SEQ ID NO:77/SEQ ID NO: 263; the CRISPR nuclease/gRNA pair of SEQ ID NO:78/SEQ ID NO: 218 or the CRISPR nuclease/gRNA pair of SEQ ID NO:78/SEQ ID NO: 264; the CRISPR nuclease/gRNA pair of SEQ ID NO:79/SEQ ID NO: 219 or the CRISPR nuclease/gRNA pair of SEQ ID NO:79/SEQ ID NO: 265; the CRISPR nuclease/gRNA pair of SEQ ID NO:80/SEQ ID NO: 220 or the CRISPR nuclease/gRNA pair of SEQ ID NO:80/SEQ ID NO: 266; the CRISPR nuclease/gRNA pair of SEQ ID NO:81/SEQ ID NO: 221 or the CRISPR nuclease/gRNA pair of SEQ ID NO:81/SEQ ID NO: 267; the CRISPR nuclease/gRNA pair of SEQ ID NO:82/SEQ ID NO: 222 or the CRISPR nuclease/gRNA pair of SEQ ID NO:82/SEQ ID NO: 268; the CRISPR nuclease/gRNA pair of SEQ ID NO:83/SEQ ID NO: 223 or the CRISPR nuclease/gRNA pair of SEQ ID NO:83/SEQ ID NO: 269; the CRISPR nuclease/gRNA pair of SEQ ID NO:84/SEQ ID NO: 224 or the CRISPR nuclease/gRNA pair of SEQ ID NO:84/SEQ ID NO: 270; the CRISPR nuclease/gRNA pair of SEQ ID NO:85/SEQ ID NO: 225 or the CRISPR nuclease/gRNA pair of SEQ ID NO:85/SEQ ID NO: 271; the CRISPR nuclease/gRNA pair of SEQ ID NO:86/SEQ ID NO: 226 or the CRISPR nuclease/gRNA pair of SEQ ID NO:86/SEQ ID NO: 272; the CRISPR nuclease/gRNA pair of SEQ ID NO:87/SEQ ID NO: 227 or the CRISPR nuclease/gRNA pair of SEQ ID NO:87/SEQ ID NO: 273; the CRISPR nuclease/gRNA pair of SEQ ID NO:88/SEQ ID NO: 228 or the CRISPR nuclease/gRNA pair of SEQ ID NO:88/SEQ ID NO: 274; the CRISPR nuclease/gRNA pair of SEQ ID NO:89/SEQ ID NO: 229 or the CRISPR nuclease/gRNA pair of SEQ ID NO:89/SEQ ID NO: 275; the CRISPR nuclease/gRNA pair of SEQ ID NO:90/SEQ ID NO: 230 or the CRISPR nuclease/gRNA pair of SEQ ID NO:90/SEQ ID NO: 276; the CRISPR nuclease/gRNA pair of SEQ ID NO:91/SEQ ID NO: 231 or the CRISPR nuclease/gRNA pair of SEQ ID NO:91/SEQ ID NO: 277; the CRISPR nuclease/gRNA pair of SEQ ID NO:92/SEQ ID NO: 232 or the CRISPR nuclease/gRNA pair of SEQ ID NO:92/SEQ ID NO: 278; the CRISPR nuclease/gRNA pair of SEQ ID NO:93/SEQ ID NO: 233 or the CRISPR nuclease/gRNA pair of SEQ ID NO:93/SEQ ID NO: 279.

Embodiment 52. The enzyme system of any one of Embodiment 46-51, wherein the nucleic acid-guided CRISPR nuclease has trans-cleavage activity.

Embodiment 53. The enzyme system of any one of Embodiment 46-52, wherein the system further comprises a co-factor for supporting activity of the nucleic acid-guided CRISPR nuclease.

Embodiment 54. The enzyme system of any one of Embodiment 46-53, wherein the system further comprises an RNase inhibitor and MgCl₂.

Embodiment 55. The enzyme system of any one of Embodiment 46-54, wherein the system further comprises a crRNA.

Embodiment 56. The enzyme system of any one of Embodiment 46-55, wherein a CRISPR nuclease/gRNA pair is selected from the group consisting of: SEQ ID NO:22/SEQ ID NO: 115 or the CRISPR nuclease/gRNA pair of SEQ ID NO:22/SEQ ID NO: 138; the CRISPR nuclease/gRNA pair of SEQ ID NO:62/SEQ ID NO: 202 or the CRISPR nuclease/gRNA pair of SEQ ID NO:62/SEQ ID NO: 248; the CRISPR nuclease/gRNA pair of SEQ ID NO:80/SEQ ID NO: 220 or the CRISPR nuclease/gRNA pair of SEQ ID NO:80/SEQ ID NO: 266; or the CRISPR nuclease/gRNA pair of SEQ ID NO:91/SEQ ID NO: 231 or the CRISPR nuclease/gRNA pair of SEQ ID NO:91/SEQ ID NO: 277.

Embodiment 57. The enzyme system of any one of Embodiment 46-56, whereupon the system exhibits trans-cleavage activity when contacting a target nucleic acid sequence.

Embodiment 58. The enzyme system of any one of Embodiment 46-56, wherein at least one nucleic acid-guided CRISPR nuclease cleaves a ribonucleic acid molecule (RNA) target nucleic acid sequence when complexed with the guide nucleic acid.

Embodiment 59. The enzyme system of any one of Embodiment 46-58, wherein the at least one nucleic acid-guided CRISPR nuclease does not cleave a ribonucleic acid molecule (RNA) target nucleic acid sequence when not complexed with the guide nucleic acid.

Embodiment 60. The enzyme system of any one of Embodiment 46-57, wherein at least one nucleic acid-guided CRISPR nuclease cleaves a deoxyribonucleic acid molecule (DNA) target nucleic acid sequence when complexed with the guide nucleic acid.

Embodiment 61. The enzyme system of Embodiment 60, wherein the at least one nucleic acid-guided CRISPR nuclease does not cleave a deoxyribonucleic acid molecule (DNA) target nucleic acid sequence when not complexed with the guide nucleic acid.

Embodiment 62. The enzyme system of any one of Embodiment 46-61, wherein the target nucleic acid sequence is a sequence from an influenza A virus, an influenza B virus, a Respiratory Syncytial Virus (RSV), or a SARS-CoV-2 virus.

Embodiment 63. The enzyme system of any one of Embodiment 46-62, wherein the target sequence is from an influenza A virus.

Embodiment 64. The enzyme system of Embodiment 63, wherein the target sequence is SEQ ID NO:373.

Embodiment 65. The enzyme system of any one of Embodiment 46-64, wherein the target sequence activates cleavage of a substrate by the nucleic acid-guided CRISPR nuclease.

Embodiment 66. The enzyme system of any one of Embodiment 46-65, wherein the nucleic acid-guided CRISPR nuclease cleaves the substrate with a sensitivity of at least 25 nM.

Embodiment 67. The enzyme system of any one of Embodiment 46-66, wherein the guide nucleic acid (gRNA) is present in the reaction mix in a concentration ranging from 10 ng/uL to 100 ng/uL.

Embodiment 68. The enzyme system of any one of Embodiment 46-67, wherein the guide nucleic acid (gRNA) is from 40 to 65 nucleotides in length.

Embodiment 69. A method of detecting a target nucleic acid, the method comprising: (a) contacting a sample with a nucleic acid-guided nuclease system comprising: a nucleic acid-guided CRISPR nuclease having at least 90% identity to SEQ ID NO:1-93, or a nucleic acid molecule encoding the nucleic acid-guided CRISPR nuclease; a guide nucleic acid for complexing with the nucleic acid-guided CRISPR nuclease, or a nucleic acid molecule encoding the guide nucleic acid; wherein the gRNA comprises a nucleic acid sequence, e.g., a gRNA sequence, and wherein the nucleic acid sequence comprises a direct repeat sequence having at least 90% homology to any one of SEQ ID NOs: 374-466, and (b) allowing the target nucleic acid to activate the nucleic acid-guided CRISPR nuclease system to cleave a detectable substrate.

Embodiment 70. The method of Embodiment 69, wherein the guide nucleic acid comprises a sequence selected from the group consisting of the sequence set forth as (i) SEQ ID NO: 115, SEQ ID NO: 202, SEQ ID NO:220, or SEQ ID NO: 138 or (ii) a nucleic acid sequence having at least 85%, at least 90%, or at least 95% homology to any of SEQ ID NO: 115, SEQ ID NO: 202, SEQ ID NO:220, or SEQ ID NO: 138.

Embodiment 71. The method of Embodiment of any one of Embodiments 69 or 70, wherein the guide nucleic acid comprises (i) a direct repeat sequence selected from the group consisting of: SEQ ID NO: 395; SEQ ID NO: 435; SEQ ID NO: 453; or SEQ ID NO: 464 or (ii) a direct repeat sequences having at least 65%, at least 85%, at least 90%, or at least 95% homology to a direct repeat sequence selected from the group consisting of: SEQ ID NO: 395; SEQ ID NO: 435; SEQ ID NO: 453; or SEQ ID NO: 464.

Embodiment 72, wherein the nucleic acid-guided nuclease system comprises a system of any one of Embodiments 46-67.

Embodiment 73. The method of any one of Embodiments 69-72, further comprising detecting the presence of the target nucleic acid by detecting the cleavable substrate cleaved by the nucleic acid-guided CRISPR nuclease system.

Embodiment 74. The method of any of Embodiments 69-73, wherein the step of allowing and optionally the step of contacting is/are performed with the nucleic acid-guided nuclease system at a temperature that is about the same as an ambient temperature surrounding the nucleic acid-guided nuclease system.

Embodiment 75. The method of any of Embodiments 69-74, wherein the step of allowing and optionally the step of contacting is/are performed without subjecting the nucleic acid-guided nuclease system to a heating step.

Embodiment 76, method of any of Embodiments 69-75, wherein the step of allowing and optionally the step of contacting is/are performed at a temperature of about 30° C. or less, e.g., at a temperature of 27.5° C. or less or at a temperature of 25° C. or less.

Embodiment 77, The method of any of Embodiments 69-76, wherein the step of detecting is performed with the nucleic acid-guided nuclease system at a temperature that is about the same as an ambient temperature surrounding the nucleic acid-guided nuclease system.

Embodiment 78. The method of any of Embodiments 69-77, wherein the step of detecting is performed without subjecting the nucleic acid-guided nuclease system to a heating step.

Embodiment 79. The method of any of Embodiments 69-78, wherein the step of detecting is performed at a temperature of about 30° C. or less, e.g., at a temperature of 27.5° C. or less or at a temperature of 25° C. or less.

Embodiment 80, the method of any of Embodiments 74-79, wherein the nuclease is JW13166 (SEQ ID NO:62).

Embodiment 81, the method of Embodiment 80, wherein the guide nucleic acid is or comprises a nucleic acid sequence having at least 85%, at least 90%, or at least 95% homology to SEQ ID NO: 202 and/or SEQ ID NO: 248.

Embodiment 82, the method of Embodiment 80, wherein the guide nucleic acid comprises at least one portion having at least 85%, at least 90%, or at least 95% nucleic acid sequence homology to SEQ ID NO: 202.

### NON-LIMITING WORKING EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. For instance, certain master mixes can in principle be combined; certain enzymes can be replaced by others with similar functionality; certain buffers can be modified depending on the enzyme to be implemented; the number of segments can be altered (e.g., duplicate segments could be implemented in certain configurations of the device and/or certain segments could in principle be combined) without altering the spirit of the invention. The present aspects are, therefore, to be considered in all respects as illustrative and not restrictive.

### Example 1—Identification of Novel Crispr Cas Enzymes with Collateral Activity

Putative enzyme candidates were identified in a sequence screen from public databases (e.g., GeneBank, SGB, and MGnify), for enzymes with (<42% amino acid sequence homology to any characterized CRISPR-Cas proteins with >65% coverage. A representative subsets of the candidates (93 candidates) were designed into Cell-Free Protein Synthesis (CFPS) compatible expression templates for downstream protein expression. FIG. 1B illustrates representative steps of the method employed to identify novel enzymes from sequences that do not share significant homology to existing enzymes. Briefly, nucleic acid-guided nucleases can be encoded on a genome within proximity to a corresponding direct repeat sequence. To identify such sequences, a proximal cut-off for a relative distance between the CRISPR arrays—which contains putative direct repeat sequence information that can be incorporated into a gRNA sequence—to that of the putative CRISPR sequence on genomic or assembled metagenomics sequence input was set. As a result, the present disclosure identified predicted CRISPR array sequence information accompanying every single candidate from the candidate pool. Based on this CRISPR array sequence proximity, two gRNAs for each candidate where the spacer sequence was either at the 3′-end or the 5′-end of the direct repeats sequence were identified.

A PUREExpress cell-free expression system was used for translation of vectors encoding the nucleic acid sequences. Briefly 25-1000 ng of vector templates, in this specific example 500 ng of template DNA comprising a nucleic acid of SEQ ID NO: 280-372 were individually expressed the PUREExpress cell free system according to the standard manufacturers protocol. The predicted molecular weight of the identified candidates is as follows:

TABLE 1

| | Size |
|---|---|
| Name | (kDa) |
| JW13101 | 146.9 |
| JW13102 | 122.9 |
| JW13103 | 120.2 |
| JW13104 | 138.2 |
| JW13105 | 95.9 |
| JW13106 | 95.0 |
| JW13107 | 134.0 |
| JW13108 | 122.2 |
| JW13110 | 126.6 |
| JW13112 | 133.7 |
| JW13113 | 132.2 |
| JW13114 | 133.3 |
| JW13115 | 134.0 |
| JW13116 | 133.3 |
| JW13117 | 129.1 |
| JW13118 | 137.0 |
| JW13119 | 137.1 |
| JW13120 | 120.0 |
| JW13121 | 125.7 |
| JW13122 | 152.2 |
| JW13123 | 125.1 |
| JW13124 | 110.1 |
| JW13125 | 168.0 |
| JW13126 | 137.0 |
| JW13127 | 133.0 |
| JW13128 | 140.2 |
| JW13129 | 102.4 |
| JW13130 | 138.2 |
| JW13131 | 134.0 |
| JW13132 | 95.9 |
| JW13133 | 134.0 |
| JW13134 | 103.8 |
| JW13135 | 121.9 |
| JW13136 | 142.9 |
| JW13137 | 93.6 |
| JW13138 | 133.3 |
| JW13139 | 129.2 |
| JW13140 | 131.6 |
| JW13141 | 129.1 |
| JW13142 | 134.6 |
| JW13143 | 152.2 |
| JW13144 | 118.0 |
| JW13145 | 125.1 |
| JW13146 | 126.6 |
| JW13147 | 125.7 |
| JW13148 | 95.0 |
| JW13149 | 137.2 |
| JW13150 | 122.5 |
| JW13153 | 131.6 |
| JW13154 | 131.9 |
| JW13155 | 134.0 |
| JW13156 | 131.7 |
| JW13157 | 115.8 |
| JW13158 | 106.2 |
| JW13159 | 127.7 |
| JW13160 | 137.1 |
| JW13161 | 124.3 |
| JW13162 | 92.2 |
| JW13163 | 125.5 |
| JW13164 | 131.8 |
| JW13165 | 132.9 |
| JW13166 | 110.0 |
| JW13167 | 11.6 |
| JW13168 | 132.1 |
| JW13169 | 136.4 |
| JW13170 | 150.3 |
| JW13171 | 156.0 |
| JW13172 | 104.8 |
| JW13173 | 146.8 |
| JW13174 | 151.8 |
| JW13175 | 130.0 |
| JW13176 | 109.4 |
| JW13177 | 107.3 |
| JW13178 | 102.8 |
| JW13179 | 148.1 |
| JW13180 | 125.3 |
| JW13181 | 132.8 |
| JW13182 | 140.1 |
| JW13183 | 116.2 |
| JW13184 | 133.9 |
| JW13185 | 133.2 |
| JW13186 | 136.3 |
| JW13187 | 130.8 |
| JW13188 | 131.8 |
| JW13189 | 130.2 |
| JW13190 | 161.7 |
| JW13191 | 127.8 |
| JW13192 | 162.8 |
| JW13193 | 128.0 |
| JW13194 | 146.8 |
| JW13195 | 106.4 |
| JW13196 | 125.2 |
| JW13197 | 151.7 |

A fluorescence-based high-throughput activity assay was then used to differentiate potential active candidate(s) from negative control samples. Briefly, an RNase Alert system was used substantially as described by the manufacturer to set up individual reactions in a 96 well plate. For SEQ ID Nos1-93, negative controls, and positive control (LwaCas13a) an amount of RNase Alert® commercial kit reagents were added to each test well as recommended by the manufacturer. The reagents used in the activity assessment are briefly listed in Table 2.

TABLE 2

| Reagents | Supplier | Cat. No. |
|---|---|---|
| Nucleic acid-guided | SEQ ID NO: 1-93 | |
| CRISPR nuclease | | |
| RNase Alert ® | IDT | 11-04-03-03 |
| murine RNase inhibitor | NEB | M0314L |
| MgCl₂ | Ambion | AM9530G |
| gRNA | IDT | Custom |
| Target RNA | IDT | Custom |
| CRISPR nuclease free water | Ambion | AM9937 |

Activity was measured by the fluorescence released upon digestion of RNase Alert RNA substrate in the system. Although a few nucleic acid-guided CRISPR nuclease/gRNA pair(s) displayed activity above the negative control levels, a limited number of nucleic acid-guided CRISPR nuclease/gRNA pairs, including JW13124 (SEQ ID NO: 22) complexed with SEQ ID NO: 115 or SEQ ID NO: 138 displayed robust activity significantly above the activity level of the positive control, LwaCas13a. See FIG. 2 (initial activity screening) and FIG. 3 (further confirmatory experiments).

The experiments below describe the evaluation of the nuclease activity of a reaction mixture comprising JW13124 (SEQ ID NO: 22) without any guide RNA, a reaction mixture comprising JW13124 (SEQ ID NO:22) without a target sequence, and a JW13124 with target and compatible gRNA comprising a spacer sequence designed to hybridize/target FauA.

The reaction set up is as follows:

TABLE 3

| | CRISPR | FluA | |
|---|---|---|---|
| | Nuclease | target | |
| | JW13124 | sequence | gRNA |
| | (SEQ ID | (SEQ ID | (SEQ Id |
| | NO: 22) | NO: 373 | NO: 115) |
| JW13124 with target and | included | included | Included |
| compatible gRNA | | | |
| JW13124 w/o guide | included | absent | included |
| JW13124 w/o target | included | included | absent |
| NTC (negative control) | absent | absent | absent |

As demonstrated by FIG. 4, the collateral activity of JW13124 was only activated in the presence of both its corresponding guide RNA and the target sequence. Activity was measure by the fluorescence released upon digestion of RNase Alert RNA substrate in the system.

FIG. 5 is a chart illustrating the results of activity confirmation of candidate JW13166 (SEQ ID NO:62). JW13166 demonstrated a superior activity profile compared to the wild-type LwaCas13a under current testing conditions. Activity was measured by the fluorescence released upon digestion of an RNase Alert RNA substrate in the system. Table 4 describes the reaction set up for testing the collateral activity of JW13166.

TABLE 4

| | CRISPR | FluA | |
|---|---|---|---|
| | Nuclease | target | |
| | JW13166 | sequence | gRNA |
| | (SEQ ID | (SEQ ID | (SEQ ID |
| | NO: 62) | NO: 373 | NO: 202) |
| JW13166 with target and | included | included | Included |
| compatible gRNA | | | |
| JW13166 w/o guide | included | absent | included |
| JW13166 w/o target | included | included | absent |
| NTC (negative control) | absent | absent | absent |

FIG. 6 is a chart illustrating that collateral (i.e., trans-cleavage) activity of JW13166 (SEQ ID NO:62) was only activated in the presence of both its corresponding guide RNA and the target sequence. Activity was measure by the fluorescence released upon digestion of RNase Alert RNA substrate in the system.

FIG. 7J is a chart illustrating the results of activity confirmation of candidate JW13184 (SEQ ID NO:80). Activity was measured by the fluorescence released upon digestion of an RNase Alert RNA substrate in the system. Table 5 describes the reaction set up for testing the collateral activity of JW13184.

TABLE 5

| | CRISPR | FluA | |
|---|---|---|---|
| | Nuclease | target | |
| | JW13184 | sequence | gRNA |
| | (SEQ ID | (SEQ ID | (SEQ ID |
| | NO: 80) | NO: 373 | NO: 220) |
| JW13184 with target and | included | included | Included |
| compatible gRNA | | | |
| JW13184 w/o guide | included | absent | included |
| JW13184 w/o target | included | included | absent |
| NTC (negative control) | absent | absent | absent |

FIG. 8 is a chart illustrating that collateral (i.e., trans-cleavage) activity of JW13184 (SEQ ID NO: 80) was preferably activated in the presence of both its corresponding guide RNA and the target sequence. Activity was measure by the fluorescence released upon digestion of RNase Alert RNA substrate in the system.

FIG. 9 is a chart illustrating the results of activity confirmation of candidate JW13195 (SEQ ID NO:91). JW13195 demonstrated a superior activity profile compared to the wild-type LwaCas13a under current testing conditions. Activity was measured by the fluorescence released upon digestion of an RNase Alert RNA substrate in the system. Table 6 describes the reaction set up for testing the collateral activity of JW13184.

TABLE 6

| | CRISPR | FluA | |
|---|---|---|---|
| | Nuclease | target | |
| | JW13195 | sequence | gRNA |
| | (SEQ ID | (SEQ ID | (SEQ ID |
| | NO: 91) | NO: 373 | NO: 231) |
| JW13195 with target and | included | included | Included |
| compatible gRNA | | | |
| JW13195 w/o guide | included | absent | included |
| JW13195 w/o target | included | included | absent |
| NTC (negative control) | absent | absent | absent |

FIG. 10 (FIG. 10) is a chart illustrating that collateral (i.e., trans-cleavage) activity of JW13195 (SEQ ID NO:91) was only activated in the presence of both its corresponding guide RNA and the target sequence. Activity was measure by the fluorescence released upon digestion of RNase Alert RNA substrate in the system.

### Example 2—Demonstration of Ambient Temperature Collateral Cleavage Activity of JW13166

The activity of CRISPR reaction mixtures incorporating either JW13166 or wild-type LwaCas13a nucleases was assessed at incubation temperatures of 25° C. and 37° C. Each reaction mixture contained: 0.32 μL of 0.5 mg/mL JW13166 or LwaCas13a; 0.28 μL of 1 μM gRNA (gRNA sequence for LwaCas13a: GAUUUAGACUACCCCAAAAACGAAGGGGACUAAAACUCCUAAAAUUCCCUUAGUCAGAG; SEQ ID NO:467; gRNA sequence for JW13166: GAACUAUAGUAGUGUAAAUUUACACUACUCUAAAACUCCUAAAAUUCCCUUAGUCAGAG; SEQ ID NO:202); 0.63 μL of 40 U/μL RNase inhibitor (NEB, M0314L); 0.23 μL of 1 M MgCl₂; 1.5 μL of 10 μM Lateral Flow Assay (LFA) RNA reporter (FAM-AAAAAUUUUU-Biotin) (Integrated DNA Technologies; Custom); and 17.04 μL of water. Following the preparation of the reaction mixture, 5 μL of target RNA (50 nM) was added and mixed thoroughly to form a 25-μL reaction mixture, resulting in a final target RNA concentration of 10 nM. For each negative control, 5 μL of water replaced the target RNA. The CRISPR reactions were carried out at both 25° C. and 37° C., with reaction times of 2 minutes and 60 minutes. Each condition was tested in duplicate. After the CRISPR reaction, 20 μL of the solution was mixed with 80 μL of LFA buffer, and the sample application wick of an LFA strip (HybriDetect, Milenia Biotec) was immersed into the reaction tube and incubated for 10 minutes. The LFA strip was then taken out of the tube, and the LFA strips were imaged using a scanner.

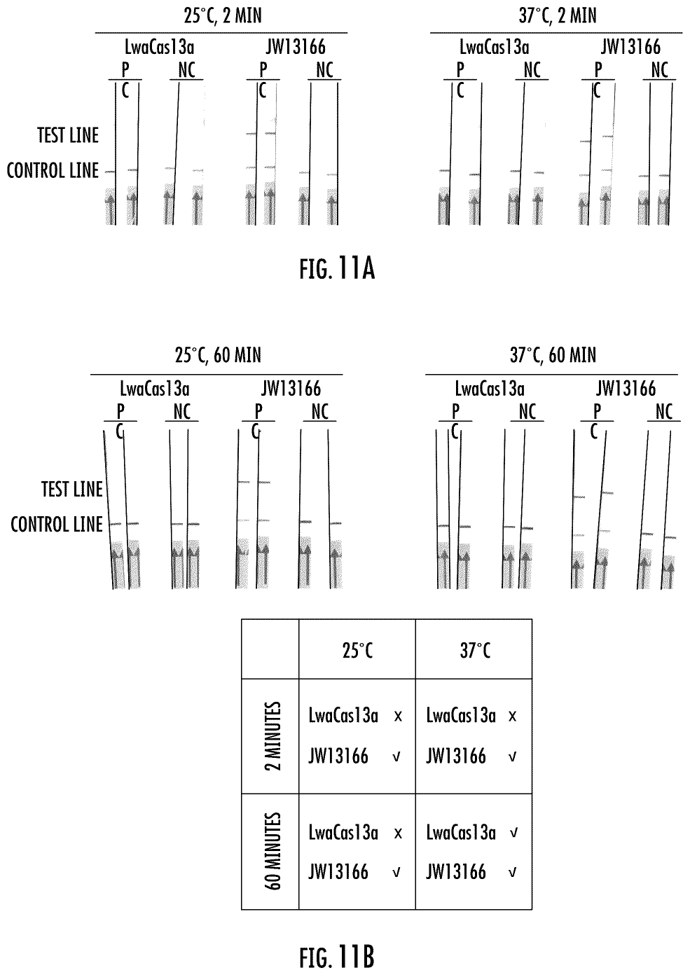

With reference to FIG. 11a and Table 7, 2-min incubations with LwaCas13a failed to produce a positive test band at either 25° C. or 37° C. indicating the absence of detectable cleavage activity at either temperature. In contrast, incubations with JW13166 exhibited strong positive test band intensities at both temperatures, indicating that JW13166 retains cleavage activity at 25° C. With reference to FIG. 11b and Table 7, LwaCas13a produced visible test bands at 37° C. after an incubation time of 60 minutes but not at 25° C., indicating that the cleavage activity of LwaCas13a is significantly inhibited at 25° C. JW13166 produced visible test bands at both 25° C. and 37° C. after a incubation times of 60 minutes. These results suggest that JW13166 exhibits significantly higher activity than the wild-type LwaCas13a even at a temperature of 37° C. Moreover, JW13166 retained activity at a temperature of 25° C., exhibiting positive cleavage activity even after an incubation time of only 2 minutes, whereas LwaCas13a did not exhibit cleavage activity at a temperature of 25° C., even after an incubation time of 60 minutes.

TABLE 7

| | Temperature | | | |
|---|---|---|---|---|
| Time | 25° C. | | 37° C. | |
| 2 Minutes | LwaCas13 | No Activity | LwaCas13 | No Activity |
| | JW13166 | Activity | JW13166 | Activity |
| 60 Minutes | LwaCas13 | No Activity | LwaCas13 | Activity |
| | JW13166 | Activity | JW13166 | Activity |

The present technology is not to be limited in terms of the particular implementations described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular implementations only, and is not intended to be limiting.

### SEQUENCES

Tables A-E describes non-limiting sequence listings provided with the disclosure.

TABLE A
Amino Acid Sequences (Enzymes)

| SEQ | | |
|---|---|---|
| ID NO: | Name | Sequence |
| 1 | JW13101 | MNQYIHADKKENKKRPNKSSIIRIMVSDFDDEYIQEIKVLYTKQGGVDTFKINKMSYDSASQKIIFEEVATQNML |
| | | SVEDSNLNFKRPMVECKNGDIYVVKPSGKVNAKGQAIEPLRSYKIHGKYLDLTEAIGKDNAELSGKKQIYLHVE |
| | | DLLGMHTTADSIDRRRLESETQRTLLSKEVMENYALIMGHEIKLNESDETYKLACSKEIYKANRFLDYRSRLLYY |
| | | YSFINHFLAGLSKGATYEYAGKSLVTEIPDGEVWQLCELEVTAYHGIYINKKTNELVNENCLEIDAIYAQMAKN |
| | | MRKTLNEFVENYNAMIEKQNELNKNKPQMKKAIVQEPIKFENFTEEKINQDLHNIVYILSDLRHKLMHFEYRYF |
| | | DLLMTGKKMGEKSEVEVSVPDRNSELPASKNQPSKDKFRKGKKLSELLNLNILKELDTFVKVKESYQTTYLETN |
| | | DKIEILGKLKTAKSIYQIYHQICQRKNGFNKFINSFFIVDGEENTEVKDCINEVFRKEIHYFETEIAKSNKDTLDDK |
| | | YKNKNQSQKTRDRMEDQIEECKRYQDDESNIDTWVAYHKDIHYSKRYKKLYSKHKTLVGNLNSAVSKGLDGQ |
| | | VIKKINDDIAIIKREMNEITKANSKSRLRYKMQMAYGFLFVEYGLKIPKFLNDFDLSHERTASKIKGYTSPEKVTQ |
| | | YLTNGEGNKDNFNLDELTKNFKKQMKFEFLKLNEDNNLIKLYILIYQLLPRELKGDFLGFVKSNYYDLKHVDFQ |
| | | TRETEAKDQFFHNMRLFEKNVKAFDLIQYSIGDEMSQLGNETFNFSQALSKIVSDDTILNSATEIPNINCLVYGSL |
| | | LKYYENAFRLSSEIEIRALIKIARKEKEVVDHSNSYSINKAHSKIEKELKERDRDKEVSFFCILEYAHKNSLNQKG |
| | | DYTDSEIYDKASNLRNKIAHCDYRNLFITNIICEAENINVKVKYLIDVSNTIGLNTVDLGNDMVNDYLMSYDKQ |
| | | MTYLVKTSEELLKESRLDKSKEKKERRDNLKNETRSHSEIYMEYEWISDYLVKLKDNHEILLKKNKTEDLKLNR |
| | | AYELIKNYNVALNNKSKIKISELTADHINNNWVNIWGVLNHELQEINGLYLYKVTPKIKKGIYLVLTDKKNYCL |
| | | NINFYTLKKVPSKDDSEMTEERYLRDISEKYYFTVGEDSMKALKDRLQTINDKCIVNYVNKIDCKNENLKGKSE |
| | | FKVIDVYNSWDKKYLSISNEESQHWSVETKRDKKYKENLILNLMEKSSFKLNLHQL |
| 2 | JW13102 | MAHNPTYLYAAYLNMARHNAYLTLSYISVAIGGKEGAEDKLLNMEALNILKKPKAEPEKLKKTEELLTKHFRF |
| | | LLPMTDNDKSAGYHEILTTAFKLLNSLRNEYTHYQHEASDFDEQFMKNMKNCFDGAKRTVKERFNLEDNDLK |
| | | HLDRFRQKANDGKKGKREYEENPTFHYKFDDENGRLTEKGLAFFICLFLEKKYATLFLTQLSGFKKGDTKGTKA |
| | | TKEVYSIYRIQLPKLRIDSKKPEMALGIDMLNELKKCPAELYDVLSTEDQKRFRIIPDEEPDTMDDTEDGEILLKR |
| | | HKNRFPYMALRYIDQNELFPNIRFQVALGTYRHHFYNKLGVDNIQRVRILQKQLKGFGRLDEIEEKRKTVWADK |
| | | IRPFEKIEKDSAEQEPYITDSYAKYIISNNKIGLKWFDRQNDYYLPELNEKGAKNEAPTCWLSIYELPALLFHSILC |
| | | NHSSDTQNIIRDYVSKGYRKLFKEVASGALQPASKEKIVNQLKTAYNLDFTRIPKELQDYLTGKPVKMDDQLNK |
| | | LCSERIAQMVSDSQKRLERIDNDLKTIKDRKSNKVGKKRFVEIKSGVLADFLAEDMLAFQSSSDNGKDKLTGMN |
| | | YQILQAALAYFGQYKDQMTEIFEKCKLINSPIEHPFLKNVMAKNATNIVDFYIVYLKAKEIYLDKCLREKQYKND |
| | | YFLHVDRQKWTERNTDFYKVLAKSYAEKLPVELPRGLFNDAIKKLLTKKHGDNPVIREALSKERCNTTFLIQTY |
| | | FKAICEDTTQEYYTFNRSYKIFNLLNDNYVGNKLQPAYYAVNEFEKRVKNIDAEIEKFDSQQHDKLHRLLHHFK |
| | | ENEKILRLYKVQDILLFLMAKRILITEMGENNIGKYQLKQIMPDSPTDILSAQTSFSITLTKNGCQKIIKQEKLKLK |
| | | NFGDFQRFVRDRRIETLFPYMKADSIDRQILEAELDAYDDKRVHIFELIHQFEESVLTKNPSILSGKTYINFAGILK |
| | | ASPEIQTEKRQQMQIIRNAFCHNTYPQKIEKNIEDIEKPEGKTDVPFSLCEKKEIPEIAEFLTESLEDLIIINEH |
| 3 | JW13103 | MDTPNFSERIPVSLQSHPYYFAHYLNMARHNAYVILEYVNRELIKPGKNLDEDNLIQSTVLKDGYFDRKPDELSH |
| | | RNRLLVQHFPFLREAENEGARTCNPVSYKLKTALAALNQWRNNASHYPLNQNHEKDFDLQPFFSFAIEACKKR |
| | | MREVFQPDDFYLLETNEKQFYTLHNENGFTEKGLYCFICFFLEKKYAFQFLAGIKGFKNTTDNKFRATLETFTEH |
| | | CCRLPKPKLDSSDIKLDMLGELSRCPAPLFDLLDIEERKKFIREPEEVKPDESGDREEVQQVLMKRYDDRFPYFAL |
| | | RYFEEKNLLKGISFHIHIGRWIKSEHTKKIMGAERDRRLLKDIRTFGELKEFSPEHAPDYWLRDGITPDDVDQFSX |
| | | PQYRIVGNRIGIKLNYNGHNRWSVPDKEINVKPDAIISTYEFLNLFLYEHLYQKKLTGLSPAEFIQDYLDRENNFL |
| | | SEFKAGHIRPVGDFSLEKRRGQGDEPDLTARRKSLQKELDRFVLKGKDLPDKIREYLLGYKQKSEKKQAKWILG |
| | | GMIKETVYWRNKAEQSPEKMRSGDMAQQLARDIIFLTPPHTVKEHKQKLNSLEYDVLQYALAYFSSNREKLYS |
| | | FFKEHQLTVKGDRAHPFLYKIRLDECQGILDFFIVYMQQKEKWLGWLDRNLKSPRLNEEEFFNTYSYFIKTDTKR |
| | | AIEMDYESCPNYLPRGIFNEPIAKAVQKAGVKIKDEDNASYALSVYSNGKTQPFYNKERYYNKGIFRMEELPEKL |
| | | QPKELLGKIQWTIKSSGKDTEEFRSLQNLKNRILNTEKEIRYVQSTDRALWIMVADLFPETFELRPDDLECIGHDL |
| | | SDDLLSRPYQMKEKVYNYTITDYLPIKRYGEFRRFLKDRRLENLLTYFEEGVPLHREALVAELEAYDLQRKNLL |
| | | EIIYRFEKLVFDRHRHELTFSGEGENQYVNHWDYLDFVARKYGLSAEVKELNSERFTELRNKMLHNQIPYQLWI |
| | | KEAIAAREENTVCGRIMGMIGEIYERMTTEIEKQMQV |
| 4 | JW13104 | MENQTQKGKGIYYYYTKNEDKHYFGSFLNLANNNIEQIIEEFRIRLSLKDEKNIKEIINNYFTDKKSYTDWERGIN |
| | | ILKEYLPVIDYLDLAITDKEFEKIDLKQKETAKRKYFRTNFSLLIDTIIDLRNFYTHYFHKPISINPDVAKFLDKNLL |
| | | NVCLDIKKQKMKTDKTKQALKDGLDKELKKLIELKKAELKEKKIKTWNITENVEGAVYNDAFNHMVYKNNAG |
| | | VTILKDYHKSILPDDKIDSELKLNFSISGLVFLLSMFLSKKEIEQFKSNLEGFKGKVIGENGEYEISKFNNSLKYMA |
| | | THWIFSYLTFKGLKQRVKNTFDKETLLMQMIDELNKVPHEVYQTLSKEQQNEFLEDINEYVQDNEENKKSMEN |
| | | SIVVHPVIRKRYDDKFNYFAIRFLDEFANFPTLKFFVTAGNFVHDKREKQIQGSMLTSDRMIKEKINVFGKLTEIA |
| | | KYKSDYFSNENTLETSEWELFPNPSYLLIQNNIPVHIDLIHNTEEAKQCQIAIDRIKCTTNPAKKRNTRKSKEEIIKII |
| | | YQKNKNIKYGDPTALLSSNELPALIYELLVNKKSGKELENIIVEKIVNQYKTIAGFEKGQNLSNSLITKKLKKSEP |
| | | NEDKINAEKIILAINRELEITENKLNIIKNNRAEFRTGAKRKHIFYSKELGQEATWIAYDLKREMPEASRKEWKGF |
| | | HHSELQKFLAFYDRNKNDAKALLNMFWNFDNDQLIGNDLNSAFREFHFDKFYEKYLIKRDEILEGFKSFISNFKD |
| | | EPKLLKKGIKDIYRVFDKRYYIIKSTNAQKEQLLSKPICLPRGIFDNKPTYIEGVKVESNSALFADWYQYTYSDKH |
| | | EFQSFYDMPRDYKEQFEKFELNNIKSIQNKKNLNKSDKFIYFRYKQDLKIKQIKSQDLFIKLMVDELFNVVFKNNI |
| | | ELNLKKLYQTSDERFKNQLIADVQKNREKGDTSDNKMNENFIWNMTIPLSLCNGQIEEPKVKLKDIGKFRKLET |
| | | DDKVIQLLEYDKSKVWKKLEIEDELENMPNSYERIRREKLLKGIQEFEHFLLEKEKFDGINHPKHFEQDLNPNFK |
| | | TYVINGVLRKNSKLNYTEIDKLLDLEHISIKDIETSAKEIHLAYFLIHVRNKFGHNQLPKLEAFELMKKYYKKNNE |
| | | ETYAEYFHKVSSQIVNEFKNSLEKHS |
| 5 | JW13105 | MKIKNENTDKKTELYSIDKYKGRDKWCFAIVLNKAQTNLTENPDLFEQTITKYDRIRKEGWFDEETKKLIYIQEN |
| | | EHKIKGEIKTLAREVLKNLRNYFSHHFYKQDCLIFPKDNIVRIIMGRAYERSEYEIKKNLKEDISIELPALFEPEGKI |
| | | TTAGIVFFTSFFVERRFLHRLMGSVQGFTKTEGEYKITRDVFAKYCLRDSYSVKAQDNDAVMFRDIIGYLSRVPT |
| | | ESFQHIKNPKKQNESQLSERKTDKFISFALKYLKDYGFEDLKGHYTAFFARSEIKKEKEDIEIKDDKKHKPHRMK |
| | | SKIEIHFDKTKEDRFYIERNNVILKIQRKGGRANILRMGIYELKYLVLLCLSGKAREAINRIDDYLNDLRNKIPHIE |
| | | NMNKEGIGEQIRSLPGFVRSQLGFVQIDDEKKKENRLDYVEKKWEKKRAESKELKLNRKGRDILRYINERCKKP |
| | | LTIDRYNRILELLVEKNIEGFYHELEELRKTGRIEKNITQALVGEKNINALHIKICKLVQDELKSLEKEDLKKYIGL |
| | | TPKEEKVVSFEEKLGRILDKPVIYKGFLRYQFFKNDKKSFARLVEEIIKEKTGGLDVPIETEYYSISTLGRFDKANK |
| | | TLYETLAMDRLCMMMARRYFLSLNKILAKRAQNIEWKKESGKEFIVFKFNMPQDTGKSISIRFSPKDYTKLYVK |
| | | NDSEFLARLCQYFFPNEKAIDYHKLYSHGINKYTNFQKEGIEAILELEEKIIKKRKINSPENYLSFEEILNQSIYNDE |
| | | EKNTLIQIRHSLLHYQILFSKNDLTKFYNVMKREGIEKIWSLVI |
| 6 | JW13106 | MIKKPANRFALPKVIISEVDSENILEFKVKYEKLARLDRFEVEAMHYDEKNEIIFDEVKVNDGLIEIVYKENNKKL |
| | | VVEIEGKAYEISGQKIGGKQRLLENRVSKTKVQLELMNHIEDGEGNKRVSRTERELIVLGDIKLYSQIVGREVKT |
| | | TKEIYLIKRFLEYRSDLLFYYGFVDNFFKVVGNEKELWKIDFNTSHSEQLIEYIKYTFNDNLKNDRQYLNEYVTN |
| | | DVQIQKDFNKVKNVFSELRHALLHFNYDFFQKLFNGQDVGFDLDIEFLNLFIDNIDKLNIETKKEFIGDEKIRLFG |
| | | ENLSLAKVYELYSHICVNRVGFNKFINSMLIKDGIENEALKDKFSTKFTGNAYYIDIHNSREYKDLYKKHKRLVI |
| | | NVSTLKDGRAIRQENKKIAELKKQMTTMTTTNSLARLECKLRLAFGFLYNEYNDYKAFKNSFDSNLKNNQFDV |
| | | SNVDTSKAYFLSTYENRKPRTRERIDKVLKEIESLELKGLIANDPLLKFILLMFVFMPQALKGEFLGFVKKYYHD |
| | | VHSIEKDTKDKEEDLIEFMPLSLKLKILGRNIRTLTLFKYALGSPINYNSTDEVFYVEGNRYGKVYKQLGISHNQE |
| | | EFDKTLTVSLLRYYSALFKLVNDFEIYSLAKGNPTVVNLDVLVDDSSSPYKFRGFYNFGNMIGQIHGLTHNQIKN |
| | | EEIMIRNKIAHFDIEMLLKKPLLAQIKLDVQRKNLIKFIEDRGDMKSVLGYDAINDFSMKMVHLRTKIKVNSDKL |
| | | QIMMGLLTNVKTPNDFYNIYKVKGIESINQHLLDVIGVTESEKKIEKEINEGNKRAVK |
| 7 | JW13107 | MENLSKEQIVETQEQNKRTWGNDPQYFGAYLNMARLNIYNVSNHLAEKFGITAIENEEQISNSFLGDKENKVFK |
| | | NKQNHIFSNLIRYLPIVKVFDFEKLPEEEKISLKDNFGKDFKELSDTLKIIFSEINEFRNDYSHYFSTEKKNKRKTKV |
| | | SDKLKDFLNINYTRAIQYTKNRFINVFSDNDFCLAEEAKLVENDNTITQDGIVFFTSMFLDRENAFQFISKIKGLK |
| | | GTQTKAFKAKREVLMAFCINLPHDKFVSENTEQAFSLELINELNKCPKTLYNVISENEKQHFLPKLEDEQIDNVL |
| | | ENSVPYTIDDYENYIENITKKVRNKNRFSYFALKFIDQKELFKNWKFQIDLGKVVLDEYTKTLQGKDETRTVVE |
| | | NAKAFGKLQHLNDEENVLNKINKSESKTHFEQFAPHYNFDNNKIGISRKEDSAIFISRNTDKKVKFNLKQPLPEAF |
| | | LSIHELPKIILLEYLENGKTEQLINDFIEINNSKILNKKFIESIKSKLTDFNTFNKRSQGRKEKSAYKKEALKYLLDR |
| | | KEKLNQILAEYNLNVKQIPTRILDYWLNVADVQEKTAISDRIKLMKRDCMDRIKAMKKGKAPKIGEMATFIAKD |
| | | IVDMIIGEEKKKKITSFYYDKMQECLALFADKEKKELFIHICNNELKLNESDGHPFLKNIILYDYRYTSDLYRRYL |
| | | EEKADNRNNLNKNDTSWLAKNFYFIEKVEVFDKRLNRMEFKPQTVVKIKDKSKIPFTLRQLEKEKSSFDTWFEN |
| | | ITKGKETTDKKKPVDLPTNLFDNYLKELLIKDLSKNNIKFNPKTNYNELFKLWWKECRKDKTQSFYNAEREYSF |
| | | EDKKLNFKINTKSKFEDYINPEFAEKVFKLKSSRREKENSTGRKLPPLMIKDLEKSMANKIGSIEKDIRILQEDDC |
| | | MMLLMFEKLVDNNLELKLENVDTLMNESIEIKQSILGKLSFDDFGDNVNKNDRIIISKNITETRKRKEYSVLKKYI |
| | | FDRRLPELFEYFETEDITMEKIKLELGTYNKAKDIVFDLIFDLESAIIKKNENRIKELNKDKNGNIQTGNIQHKPYL |
| | | FWLLEKNLITEQEFTFLNMVRNTFSHNQFPQKKTMELFIKNWNKNEYAKQILTVYTEKINNLIEKVIDI |
| 8 | JW13108 | MESFPDYKPLFAAYLNMARNNCFIALSHIAEKANKGGGMNEDAMQECSVITLLANNKKPDESLKVEEMIKYHF |
| | | PFTKSTKRIDPVPSDYAGLIISFIKQLNELRNFHTHYLHRPYATDANVIAQLQFLFDTSRREVRKRFALKEEDVKH |
| | | LVRKTITIINGEKVVVEKDNYPYAFVNKEGQFTTKGIAFFITLFLEKKHAYLFLKQLRGFKNSSSKQNKATLETYC |
| | | FYHIRVPQPRLQSQDTKVGYLLDVLEELKRCPKELYSHLSEHDRMRFTFVNDIDATDEQPESILKRNKDRFAHLA |
| | | LRYFEHTGSLKELQFYYDLGKYYFKVYEKSVDSEIRLRRLHKYMKGFGRLNDFGLENMPPLWQQLVKYTDQLS |
| | | EGYAEPYIVYTIPHYHIVNNQIGIWINNGFIPPLLSPVSSPEPQMWLSIYELPAIMFYHLLTKKRKEYKSVLQILSNH |
| | | RKAIHLLFKDIKDSKLLPDAVETFEAELRQRGLHRNHIPKPLLEYLLNESPGTPVAAKAENRLIKLQNETSFLFDKI |
| | | DIDLEKSKEKAGGKYYKPIQTGKIAQFLAKDMLFFQPPINKLKGKATGTLFQVLQARLAFYGRDKHLLEDIYREC |
| | | NLIQSNNPHPFLQGLPITDSLIDYYRNYLVRRNTYLEKCLKKCMQGKTGSQELHFLHLGEREKRAGNDYYKGLA |
| | | GKFIKMPLNLPRGLFMEELLKVFSAENKTMQDIADNSRTNTAFLIKEYLKNVLDDDTQDFYNWKRTYAVFNKY |
| | | YDNRKPQSRDSLIERYFDTSVLSTEAEKIKLEIVKKQRVDIEASENIKRNYRYYSENEKQIRHTKSCDIVLFLIIQDL |
| | | FPSLEQSVFKGLAINQLKLSDILPDAEKGILSLPIEIRLPLYNKFIIQRGIKLKNYGDFRRFLKDRRLQSLLPYFEEESI |
| | | DRVIIEKELQDYEIARIEIHRLVLEFEKKVITENKITYENSEINYVDHEKVLTAFFQNEPSSNKKELIRDIRNKFSHN |
| | | EYPDANKFKGLIESDSLNTTVAKKFIQIAKQYYQFEIAPAPPLLVLSPTSTTIL |
| 9 | JW13110 | MSIMNIITVLNHIHQIAGLKRNELGDDDNIYSHPVLKYIAPENNSTTLPEKRDLIKELLLEYLPFLEAMKDCYKEV |
| | | KVEKDKRNNKYVKDKKQVFIRNYSDIYCIYWTLYNIFSTLINYRDKSCHYRFIPSKDQEEYSKRTEYDTVRIINK |
| | | YYTYALRKTKEKYGYTTEQLSFIQNNRYTDDKKVNFNFFLSMQSSNNNLSHVGVAQLICLFLQKKDVDNFLEN |
| | | LPIYDNRYCLGIDEHMIIKRSLALHSIKLPKERLNSEKKDMAIAMDMLNELKRCPKELFNTLPFDKQERFRIISSDH |
| | | KEVLLMRSTDRFVPLILQYIDCNKKFKDIRFHVNMGKLRYLFSPNKLCIDNQERVRMLEHLLNGYGRIDEVEYL |
| | | RKAPNGTFGNTNIPIKGFGEIERDSADSNNYPYIVDTHTHYILNNNKVEFCFCEDSKNKNVFPTIEKEINGKWYVE |
| | | KTAPHCRISFFEIPAMAFHMFLCGSEKTEKLIKDVYNNYISLFNALKNGALTEENYKDFNIPECDMPKKILDFLKG |
| | | KAEGKSIDGYVKNILKKELKDTRKRIEQAPSEKDQMPSKRNKIGTKSYKRVLTGKLADFLAKDIVKYQPTLCAG |
| | | DDYGIDRMTGMNYRIMQATIATYNDDDDFDTFYNVFKMAGLVDGDERKNHPFLKKVLLSKPQNTVAFNIKYL |
| | | KEKEIYLRNIEKKNIIPPFVNRERNKWCERDCEYYRILGENLLDLPIELPRQMFYKDIRAQLEPLMPEIDFSQTNTT |
| | | YLIAEYLKRVRGDDFQEFYSWERNYRYIDFLKCIRDDNGRICAQYTTTEERESLWEDSKNRTAEYKRSEEQKNE |
| | | KKDKNFEDSLEERISSSHIKFQNTEKHIRRYKVQDALLFMIAEKQFGKYLDDIKAKNFKLKDIMPDTDKGILSET |
| | | MPIDFTFTKKNIKYNIHAKNLKFKEYGDLFILANDKRLESLWKILATDTVNKEEISEEFAKYDFCRPDITKLVLDF |
| | | EKDVLEKYPEIIEEAEKEYKKFDFKFILDWLINKKKLNYGIRNKLLLIRNAFNHNSYPDKKYAEVRELPEIATNLK |
| | | KTFDENAKKYE |
| 10 | JW13112 | MRIVKPYGRSHVEPAATGSTRRVLRLRPPNDKDAKDIEAFARSHEELVIAQWISALDKIATKPSGKDGASDAQR |
| | | AFRERLGNAAWTLLETKGLLPGLVTRREHLTMLWRAKVHPYGSTPYRPKRGKPAPSLKGRRYERFAGQLEVDK |
| | | VDAAAIAASIHAHLHGTAVPAVRPGLIATRARSIAENVLRPPQRLPNEERSWSDDDERAYAAAGDVAREIQELAI |
| | | AIEEDASQRRRGKPLTRSPFALRDAAPALRGQYGRLLVDASGDALPIAQARAAFPGLFGLHMAVRDCYSRILKD |
| | | HRKSSVVRILPRDMEELLRLVGAKLTNRDLNAMVRLGKVIHYQSSLGVEDSPENVAANWPADVSGSEFWTSAG |
| | | QAQIKRNEAFVRVWRHVLALAQRTLTDWIDPQGKTQGDILLANPPFNRDAYNRKSDLLFGKRASLFKGVETFE |
| | | NVVLTLARRETAALRHGSFHFKGLGGFTKVLAEVPKTDASVLAAVRTLWEDDLSERAQRLRQTLRGAHVEYFL |
| | | DEAQGRRLLSAVLADSGPATLPLPRFRRILQRAHNAWAKETDGPGLPEPANRMDIKTYPALHCRYVALKHVYE |
| | | RPFRHWLEQRDAASLNRHIDRAVDRGTKAARDLNAGDDEDRRRIIVARAASLGRLAEDDNVQDFFFELSAETA |
| | | GEMRVQRGYASDAEAAREQAGYIEDLKCDVLALTFADYLKAAGLDFIRILAPETPRPDNAAYNLDAIEAPEIDTP |
| | | GEDWQRVLYFILHLVPVEDVGRLLHQTRKWEILAGDETLSPSVRDRLRGIQLALELYLDMHDAKFEGGAALVG |
| | | TGAFKDLFETARIFDDIFPSQPGSAQTADDERRVPKRGLCEIMRFGHRPALQPIFSRYRVTEREVEEVRAMEWPV |
| | | DGTSEIARLQDRRERLHKGWTENKSGLSGDDLRAYVAALREVAAHRHLAAHVTLTNHVRLHRLLMAVLGRLV |
| | | DYSGLWERDLYFVTLALIHRAGRRPADVLPADGLRFLANGQIVEAVRRLEPDLQSRLLDCLGDIDKNGKVRIRN |
| | | RFAHFNMLQPGNLPVDLTRCVNDARELMAYDRKLRNAVTQSVKELLAREGLMLGWAMGADHRLSAATLATR |
| | | QAQHLGKTRRGEKGWHERKLRPIPILEDLHGRRFVDMAAALFGGTRLEPERSLLDHPLDRIEWNPTPKNRYGSR |
| | | PTSGQGRRNVVPR |
| 11 | JW13113 | MENQELKTRTMKSEPYYYGAFLNMARLNIFNISNHLSNKLNILPTLSSEEHIANAFFTDKNTKIKWEHTYDILRRF |
| | | IPIVKVFDTESLPKGEVGNNTGKDFSKMSDTLKIIFKELNEFRNDYSHYYSTEKEDKRKITISDELANFLNENFKR |
| | | AIAYTKKRFKGVFTEKDFELANNIQLFNKDKEITEKGLAFLTSIFLEREYAFQFISKIEGLKGTQKSEYRATREVFM |
| | | AYCVNLPHDKFISEDAKQSFSLDIISELNRCPQTLFNVITEKEQEKFRPTINQQEKNNIINNSVPYDIEDYEEYVNSI |
| | | TKKIRYDNRFPFFALKFIDETQVFEKIRFQIDLGEILLDEYTKQLANNEEKRQVVQNAKAFGRLNDFIDENNVLEN |
| | | INKQNGSASFIQYAPNYNFDNNKIGIDTTGKRIMPILTKQTDNNKKVKNKLKQPLPKAFLSIHELPKIILLEYLEKG |
| | | KAEKLINDFLLINESQLLNYKYIEEIKNKLNNFDVFQKRSQRKKLQTAYNKTNIEELQSRKEELNKILKEYKLND |
| | | KQIPTRILEYWLNIEDVTPNEAISDRIKLMKRDCVDRLRDIKKGKAPKIGEMATFIAKDIVDMIISKDIKQKISSFY |
| | | YDKIQECLALYNVSEKRDLFLTICNELRLLDADKGHPFLKNINLNRINYTSDFYVKYLQEKGHKLIKETNYRTGK |
| | | LVEKDKSWMFLNFYYLKKNETLNKMMTIVQLPDDKSKLPFTIAQLDKPKNTFEEWINNITKGKTKTDKEKPIDL |
| | | PTNIFDKEIEEILKNKLSEEKIAFTENANYNQLFKLWWTDCRKDNVQKFYDAEREYVIYDEHVKFIPNTKPKFEN |
| | | YYNESFPIVLTRLKRDREEARKLNRKLPPIEKSQVEKVFKQAIGSTEKEIRLLQEEDRIMLLMFEQLLETDNNLNL |
| | | KLNNAESLLNEQITIKEKISLKLSFNEQEDKKEIIKTIIDKRKRKDFSILRKFKYDKRLPELCEYFETDDISHSDLKK |
| | | ELDEYNKAKEQILANAFMLEKTIIEKDKDGIKALFLNDNGEKKYGNVQHKPYLTWLKNKGLINDNEYLFINMV |
| | | RNTFSHNQFPQKRTIQIFIDKNRGNPITFAETIKNAYNKKIEEIIVKIK |
| 12 | JW13114 | METQENELFENRRRTLADDPQYFGAYLNLARLNIFSINNYLAKEFQQTELRDDELIQNGFICIPSQKNRNDNKLFE |
| | | RLIRLMPVIKQFDAARLPNEEQKSGQAEGKDIKTMAKTLKALFKDMQEFRNDYTHFYATDKQECRKLTVSSET |
| | | ATFLKASFARAIEYSKVRFKDVLAEDDYSLVAGKELVEDENTITTEGLVFFTALFLDRENAFQFIGKVQGLKGTQ |
| | | FKSFIATREVLMAYCVRLPHDKLISDNPIQALLFDILNELNKCPKKLYNVLSEEVKKEFRPELSESGIENMLANSF |
| | | NDEEREKLLDEIEYKEYIEQLTIRVRHSNRFHYFAMRFIDEQELWPGIRFQIDLGKYTLTQYDKQVLGESIPRSVV |
| | | ENTRAFGRLSNFLTADPDSLEQRIDVDHRANGFEQFAPHYNADINKIGIAFTEKEQLPYVVPTKNAETNRVKYSF |
| | | KQPQPDAFISLHELAKAILLEQLQPGESEKLIRAFCSTAENKLFNLEFIESIKWKLPDWKPFQKQCDTKKSRAYITK |
| | | KKMFAYAPRDRKKLLDELLAPYGLNWKQVPERILNYWLNIEEVHDARSIKERILLMKRDGLKRLKQLEKHRKD |
| | | PSIRIPKVGEMATFLARDIVDMMIDKTKKQKITSIYYDKMQECLAFYADAEKKRQFIGLVRELNLNAPDGHPFLK |
| | | NLNLESIRKTSEFYKLYLMEKVEKMIENGYKSNGDKRFKNIAWIIRVFEEKQKNEKNGKDDTIYRIPNNRNDIPY |
| | | TIRQWEKETSTLEEWLHTINHGKQEGDRKRPIDLPTDLFDARLCELMKGQMQENGQTFADTAKYNELLKQWW |
| | | SLREDETQPFYNGARHYVIKEEAVCFMPGAQPRLTSCYADAVEEAYNRLSAERNIARRHNKQLPLIQKQKVEKV |
| | | FKHTLYDTEREIRLLGEQDRILLLMAEQLTPGRETLKLKTVSEELTRPREIKKEFPYISNYNAQGEKLPKDEPKPKI |
| | | KVTVTGIMQLKNSNDLNRFAHDRRMPELVAYLPNATIDADTLRYELMDYNKARLTVFDAVFRLEKAIIDQDRE |
| | | GVITLFTDEQGEAKQGRMQHKPYLQWLLNKGIITQPESSYLNMLRNSFSHNQVPHIAVALNGTEVVPVKNIASTI |
| | | AGAYKQKIETIIGKIG |
| 13 | JW13115 | MNNNILEQSTSGSEKRASTRTLEADPQYFGVYINMARHNVFQIINALTHKFSKQIENAKPINDDENIANDYLINIG |
| | | KGTDKSPTIIEVLNPAHPLHRLYGEHVFRALKRYLPLVTIFEVPSINTEDIIKDTDDILPVLFQDLYEFITLLFYELV |
| | | KFRDAYTHALALDEQGNIVPRKIDLHPNLRHYFEHLFYKAPDYTLRRFRSTQEKIAHGFHEKDFALLKAHNLYQ |
| | | DPHSNELTTQGLFFFTTLFLEQKYAYQFLKRFNGFKNETIQPFKATLRVFTAFTVKLPNDKLASDHPGQSFLLDM |
| | | LNELNKIPQSLFKHLTPKDQEMFERINDEALTNILKNTDEASFKDVDDLLGKMAPLIRREDRFPYFALWFLEQTK |
| | | AFNHIRFQISIGKIAYSPYDKTIIGETSPRIIKFDIHGFGSLAEFSDEDTLLTTLKRQLPAGSPHASLTFSQYAPHYHIK |
| | | NNKIGILVDYPSGDRPLSNITQQPDGKIKVNHIKPTAFISINELPKLVVLALLGTNKDNKPIGNDIERLIRSFITKFKN |
| | | QLFHLAPLTKIKNGLTFSPETFTRRMDKDKMVKGRIPKSELAKREEYKIYLEKRKAVLQGTTDLKLNVNQLPTKI |
| | | QNYLLNIDEVSDEKSIHLRIKTMIKEVEYRLKKIDRNKLPKVGDIATYIARDMLDMVADVRTKSRITSAYYSQIQ |
| | | RALAYFALDKTRLIDLCDELGLFNHQTGHVFLIREIFLNNHHILGVYREYLTKKQNWIDQNLIVKIQERGKRLTK |
| | | YILPTNRPLPYKMKQSINKGFEFNAWLQHKSTMPIDLPTTLFDESLTSLLKKTIAQKLGIASKSEDKFGRLLDRLL |
| | | KQDTQPFYKFERIYRKPFSQEEPEPFNILKLTHTALKQRFGKPAEVNEKIIRHHQRKDQIVRLMCEHLFKNNSELA |
| | | FEGEFLLENLHPASRNNPLDAPMVFEQTIPHTHKKIVAQDSNKWIQEVKKTNNRNKAWYRWTLKDFGTFRKIIH |
| | | DRRLPALVNSITGDEIDFDLVQFELAAYDRARETIFDQLFLLEQFIADKDMSYIKQHKAIERMQDGKGFDEVQFN |
| | | TYADWLSDKGLLTPDEINGLKEIRNRFSHNQWPGISKLGIQPISQEQQSAFVTHLNIKGWVNEHHTPLAMFMEKL |
| | | VINWVSKAIT |
| 14 | JW13116 | MGVEKNKVFESVIMNFDQERKYGFIEYKETNNLFFHMENVKNPKEIVKGAKVRFEIYENPKPKKQNQRFSAINV |
| | | EVITDETHKEAKIQKNEFKTFDQFTKELQETQKVNGETKKEHITKNKHTNVKAAGVKSVFAVDDGNVLITSFGR |
| | | GNAADIETLKSDDDKTINLTETENQKKYVVTNKRSNVKGLADNPTKVESIIPGETQIGFKSILEKHFFGRTFNDNI |
| | | HIQIIHNILDIKKILAVHTNNIVYALDNIHERGRENSAEKPIDMIGAGGISTSKEYEQYCSEKSDYEDNFLKQLINNE |
| | | RIAYFGNAFFKDEGNKKVMRTEKEIYYILGMLNEVRNVSTHFTEEDNRDWAKANLYNLSNRLKVGSKEVLNQL |
| | | YKEKIDKIDANGFVNKGCKRDFSILFKSLNLTTDKDKGELVVGFYDFSIRKNYKNIGFSIKTLREYMLKISNSTLC |
| | | ADTISNNAIRPKAYKLYDFIIWHYYMNKPDKINDFVEKLRTQNKNDEKIKLYYDEAVCLLSELGREIHTMTSCVH |
| | | NIENTSYEITDKKQKEYYKMQINSLNSADKVSDFSKVIYLVTLFLDGKEINDLLTTLINKFDNIASLLSVLEKQSG |
| | | KKVEFVENYSFFNSSNLLKEKTLNKSENYTCKIVEELREINSFARMTGDCKIRKSAFEDASQLLGYHDKTVNNLF |
| | | EVLRLKELESKDWKKRTDDEQQEYDRLLNKHHYFKSGKKLPDTGLRNFIINNVIESRRFNYIVRYADPKKIRKCT |
| | | ENNELLKFAFKDVPDSQVDRYYNICVTNKITNATREEKIERLVDIIKSMNLSKVATVKQRDKQDNVEKQKQLAI |
| | | MSLYLNILYQIAKNLVYVNSRYVMAFHSLERDSQMLFDAYYDVKRGYCDLSTVLLFGVDDLQNRNRGSYKYL |
| | | RDNRRSNKDVIETFGDFKGKVSKVVEKKNQGLTNEIYDSLCNVAGTTKTEVQNEIKSILKSNGLDESASSYLSHK |
| | | LVNKVHSYKYLKQNLDCADNTMINQFRNNVAHLNTIRNMDGIENVTGITSYFQIYHYLMQKALYKEFKKCREN |
| | | AVRKWIPYITENAEPKYVYWNKKEQQEVEVSFNPKIFGYMENIKNHSNTYCKDFVKALCAPFAYNLPRFKNLSI |
| | | EELFDMHELSEEPKESMKLTD |
| 15 | JW13117 | MEKSKLDNEMTFAEDAYYFAHYANMARHNAYLILNHINKSIFGNESTLKEENLQIAIIKILDPKNGADHHAKAIK |
| | | LLNHHFPFIACYYKEDIKKAVENSAYKEMNDKKLDHLKEPLTLKQILKTLTGIRNELNHYGYSRKEHPIPFFQDL |
| | | FNDSIQLAKKRMIEFNETRFNSIQDSPYHKMVDPKDSVLTDTGKLFFLCLFLDKKNAFEMLAKVKGFKNRTDEK |
| | | YRATLECYTQLCCKTPYPKLESSDISLDILNELNRCPNALYQVLSEEDKLKFKKEPNEETAEELNPIMKRYDDRS |
| | | AYLALRYFDRTKCLDNITFHLQLGRKVMGEPHKKVFDGKERTHQILKEMRVHDKLSTYETEEAQTFFQSKIESN |
| | | EVELFAPHYRMVGNRIGLSFGTPSEKKEGYSIPINNQPAPQAILSTSELGALYIYNRLYKENKITQSPKNLMSTYL |
| | | QTMNRFIKDWKNDGIKPETSSNKIIKERNQERTKQTINELKRRTDHLQKLLDEKYAGLKVKHLPDICREHLLNYQ |
| | | SDDNIQQAIDFLKKTLSDTNFKKKQLEQFERNMVYRSSNSKQLKIGEIAQELARDIVFFLKPIKKEITNRNKEKVT |
| | | VFQKINNQDFDILQNRLAYFSLYKEELCSFFDDLLKKKHMKHPFLENVKAETKWSNGLIEFYLKYLEHKLKWLN |
| | | TYTKNAPNNTTRPNYTTSNRINKNRSTPNKKDEIEEKEIDPEKIKETFIEKYNYFLKVKTSSPKNYQGKAIFIPVGL |
| | | FNDAITKAMKESGYQVEDDEKTPNAAYWIKSYFHGATQPFYQLPRYYNRAKKDEERDLVEAALLKKEIKNYYV |
| | | KANKKTQNDFKQLAKRIRKQEEEILTQQTSDRVLFMMATDLLNGGSDGRQTIECEQINTLGFNLGENKILDENR |
| | | KVSMNIYGRKVVSELPIKRYGEFRRFMKDRRLENLINYFPEGKDIELGVLKSGEVEAVGERFSSSTLANELEFFDS |
| | | QRWLLMKSIYEFEKKIYTHFFDVLTKNVPEKEMMENNYIAHYHYIECAKKLELSVSELEKDDFKELRNKIQHNEI |
| | | PYSQWIKNRISTKDGHLITKQIVELVLSIYQQMNNELDQLIRK |
| 16 | JW13118 | MNHTIGKGISYNPFKHQDKHYFGSFFNLADNNIKDVFNEIAQRLNHKDSNPKNIINHYTDKNISLVDFEKFVSLLS |
| | | NYFPIVNEIDQINCDAKGSNGNYKPKTKEERLGYFKKTSYSLLEAIDKLRNYYTHYHHEPIEIDLNLFPFLDHVLL |
| | | KTALNTKKNYLKKGKTKELIKDNLKQQFDFIFNIKVEEYLKEKNPTSKKIERAGGKIENFNKSLVEEITSAIYNDA |
| | | IKDFIYSKGENAELSNRKKTSFSDKDYFYYNDDDHFSLPISSSGIVFLLSCFLNRKEIESLKDNITGYKGKIPNSELT |
| | | LKHNGIRFMTTHRIYSHWCYKGLKTKIRTSEKATRETLLLQMVDELSKVPDVVYQNISDDLKKTFIKDLNEYYID |
| | | NQENQENLYNSKVVHPVIRKRYEDKFNYFAVRFLDEFLDFPTLRFQVHLGNYLEHSTSKNIGNSKTNREIKKKIF |
| | | VFGKLSEVNKLKNDFFEKNKQNDTSWQFFPNPSYHFPMENSNDLKKANKIGIYINHDKSINKFKHQTQKQTHEN |
| | | KIKLIQKVIGDKSKIKIGQPIAYLSMNDIHSILFEALKNPKIKESGKIDGEHIEYKITHQINKQIKEIQNKNENAKIIK |
| | | NQQSKQETDFNIDKLIKDVNKEITISEDKIQTLKENKKQYQNYQRSKKSEKHNNSRKRNHILYNSEKGEIATWLA |
| | | NDIKRFFSKDFKDKWKGHHHSNFQRNLAYYNTQKKEVELLLKGLDYRKTIPMLDFSKNSFLDFYRKYLEKRKQ |
| | | YFKNLLSELKNHKSGKHDSIEKLKKKCFTHFKKRNYRNKTIEDKIQNMLANPIFIKRGFMDTKPTMIPGKNFNQN |
| | | KSDFADWFVAFKEFSEYQKFYDTNLYPIDLSQKSKSATKKINTEIYKQKKNDWAIWQMTKYIFKDIFQQDSQNIS |
| | | LSEIYQSRQEPISNAKNTATDKRKQNSIWDSTIDLQLNRKIKISDVKLKDIGNFRKYENDQRINTFLDYGDFSDW |
| | | MAYLPNNWQENFEYKPINVIDIQLDDYEKIRQNVLLQEVQNLEREIYNNVSNKNEILVQDSKGNKNPNFKAYIL |
| | | KGLLKQIKNLDITNFKLPQNAKEFDTLTFKHLQTLSVIEQKTTLLILLRNKFAHNQLPNKAAYEFCQTILEREKSQ |
| | | TYAHYYSELFKTLKKELTDI |
| 17 | JW13119 | MMETIKQEIGKEYNPFKTEDKHYFGGFFNLADNNIKEAFNEVSIRLSQKGASPKTLIEKYTKENVSLVDYERFVIL |
| | | LSDYFPIIKEIDQINKDEKDNKGILIPKTKNDRIKDFEVILVSMIDAIENLRNYYTHFYHESIEIDNKMFYFLDKVLF |
| | | KTVLDTKRNYLKEDKTKEMIKNTLKEDFNKIFNLKVDEYLTKDNLTSKKIKKARKYGNSFDIGLTKEITASIYND |
| | | AIKYFTYDKNEKSILSDSRKTAFNERDYFYRDKNFDLPISSSGIIFLLSCFLNRKEIEDLKSNIKGYKGKVTKNEEPT |
| | | LEKNSIRFMTTHRIYSIWHYKGLKNKIKTSESATKETLLMQMIDEVSKVPNVVYQNLSKDLKDSFIEDWNEYYK |
| | | HNEENTENTINSKVIHSIVRRRYKDKFNYFAIRFLDEFVNFPSLRFQVYLGNYLVDSRPKESANVITEREIKKKIFV |
| | | FGKLNEVNQLKNDFFSKIEEDKLETNLEVFPNPSYHFPMENSEELKSANKIGIYLSDIENLLEPFKKHLNKNTSQE |
| | | KISLIEDITQQNENIKIGQPLAYLSMNDIHSVIFEVLKNPIIKDGKIDGKSVEKKIKNQIYKQINEIKNKKEESKIIKNF |
| | | RKKTESDLDLKKMIDDLIKEIKITDDKIKKLDENKKEYFDYQKNKTAKKRNYILQNSEKGEIATWLANDIKRFFP |
| | | KEFKEKWKGYHHRELQLSIAYYDTQKQNVKLLLYGLDYRKEIPMLDFSKPNFLDFYEYYLKKRKPQLYNLLSD |
| | | LEKLERGESINKEMLLSKYFKLFRKSNYQNKSLDDKINTALANPIFIERGFMDGNPTVIPNTTIKGNESRFANWFV |
| | | AYKQFDEYQKFYNTEEYFLELKDKTPNEIKKIQSQIYTQKKNDWAIWKMISYIFKDIFNQDLQNVSLSELYQNRE |
| | | QRLENEKKSKEGERKQNFFWNKTVDLQLNNKINIPEVKLKDIGNFRKYEKDERIKVFLSYNDITDWMAYLPND |
| | | WQEKHTIKPINVIDIQIDKYEKIRQHHLLKEVQNLESEIYNKAINKEELLQEDIKGNKNPNFKKYIVNGLLKQIKG |
| | | LNVENFKIPNSDKEFNALSEETLRSYTELEQKVTLLVLIRNKFAHNQLPNKDVYAFTQSIRKRQKNETYAEYYFN |
| | | LFEKIRGEI |
| 18 | JW13120 | MDTPNFSERIPVSLQSHPYYFAHYLNMARHNAYVILEYVNRELIKPGKNLDEDNLIQSTVLKDGYFDRKPDELSH |
| | | RNRLLVQHFPFLREAENEGARTCNPVSYKLKTALAALNQWRNNASHYPLNQNHEKDFDLQPFFSFAIEACKKR |
| | | MREVFQPDDFYLLETNEKQFYTLHNENGFTEKGLYCFICFFLEKKYAFQFLAGIKGFKNTTDNKFRATLETFTEH |
| | | CCRLPKPKLDSSDIKLDMLGELSRCPAPLFDLLDIEERKKFIREPEEVKPDESGDREEVQQVLMKRYDDRFPYFAL |
| | | RYFEEKNLLKGISFHIHIGRWIKSEHTKKIMGAERDRRLLKDIRTFGELKEFSPEHAPDYWLRDGITPDDVDQFSP |
| | | QYRIVGNRIGIKLNYNGHNRWSVPDKEINVKPDAIISTYEFLNLFLYEHLYQKKLTGLSPAEFIQDYLDRENNFLS |
| | | EFKAGHIRPVGDFSLEKRRGQGDEPDLTARRKSLQKELDRFVLKGKDLPDKIREYLLGYKQKSEKKQAKWILGG |
| | | MIKETVYWRNKAEQSPEKMRSGDMAQQLARDIIFLTPPHTVKEHKQKLNSLEYDVLQYALAYFSSNREKLYSFF |
| | | KEHQLTVKGDRAHPFLYKIRLDECQGILDFFIVYMQQKEKWLGWLDRNLKSPRLNEEEFFNTYSYFIKTDTKRAI |
| | | EMDYESCPNYLPRGIFNEPIAKALQKAGVKIKDEDNASYALSVYSNGKTQPFYNKERYYNKGIFRMEELPEKLQ |
| | | PKELLGKIQWTIKSSGKDTEEFRSLQNLKNRILNTEKEIRYVQSTDRALWIMVADLFPETFELRPDDLECIGHDLS |
| | | DDLLSRPYQMKEKVYNYTITDYLPIKRYGEFRRFLKDRRLENLLTYFEEGVPLHREALVAELEAYDLQRKNLLEI |
| | | IYRFEKLVFDRHRHELTFSGEGENQYVNHWDYLDFVARKYGLSAEVKELNSERFTELRNKMLHNQIPYQLWIK |
| | | EAIAAREENTVCGRIMGMIGEIYERMTTEIEKQMQV |
| 19 | JW13121 | MFDNEQKNLEKEPYWGVFLNQARLNAYIALRDISERLEENAADEDSLSEWPVLKYLDNDTDAVKSRRIFDLVE |
| | | KHFSMLKIIYGGEKEGDLVKRSKEYKIILKCLFRALNFYRNKFCHMYSGNRARKYNEKELIKYLEDCFDASVRKI |
| | | KELRRLDEKDVLHLRRKIAEGKDANKRVIDNPQFRYPFKNEKGELNEKGLYFLASIFLDKKEAHEFLKKQEYFK |
| | | NDSEPKYRATLESFYHYRIKLPRPVIESDVDKNGLALDMLNELKKCPKELFDLLSKEQQEKFRVVDSEDADEEG |
| | | NEILMRRYSDRFPYLALRYCDENQVFERIRFQIDLGRYYFKFYPKETIDGKTQQRSLDKRLKIFGRIKDVKSKVEQ |
| | | EWSGIIKSPDTIEENPNEPYKLKTTPRYNIVDNQIGFVITGDKNLPDVKRPDGRIELEKPDGWLSIYELPGMLFHGL |
| | | KYGFDKTERMIKIYIEKQRKICKEICEKGTITPDDGESMPEALKGGAKAAKRNYSEKKLERMLQDTEQRIRAIQT |
| | | TQKRMDEPGNKPGKKKFFDIRAGKLADFLARDIMALQRFDPAKHGKDKLTAINFQVLQATLAFYGAKKDVIED |
| | | MFKGIGLLEGDNPHPFLNQIDPAQYNSIAGFYQAYLQKKRSYLEDYRKEEEYDEQFLRPKRQRYAQEKREIKTV |
| | | ARQLLDNPVNVPKNFFKKEIEEFVFSQDPSLKKSKMNTAYMIQALFEKHYGRQQPFYSYNRTYPVVSKAIEYGK |
| | | KGKNKKIAKVLMAIEPKLNYMEIKKIVNEMPDGQYEPENLKRNLYEGYKDYEKDERIIRRCKVQDVVSFMMVE |
| | | ETLKDQLDFNGNVLTLEKITPWEASPFKKPVLCHTIISIPFNTKGGHTDKDYVDFIKNNFEGSYDCEPNKIILKYK |
| | | VTSKDTKLKDIGKYRMYSHDRRLPGLLIWKYRPNDQNGNEIKFTEIEQEIKAFERRRIEIAQCLYTLEKKVIDSWF |
| | | TQDELGEEHIPFNKVIDVIKAKMPNFEDKCNVLLKIRNAINHNQFPVYEQAIQTAPGKEIAGKMLRITESYIEQIM |
| | | AKIDPDFGRTEDAESSR |
| 20 | JW13122 | MPXXNNQKKKADSIKRXSPSPSRINGVAKPRPLVVDENNKHVLGGYFGLGLNNFYKATLSIFARTGIKIASKNGT |
| | | VLYPEEKIGQVIRVLRRSISDTSGNLTHEEKVWSNAFKLSSSQEMRLQKLIFHHFPILGPIMADEGAYKASTNKKS |
| | | KVTDAYSMTYGVTLEECLDVLGDLAQTLVDCRNSYTHFYPYNSKESTADQLALQSKTARYLDKVFTASRRLDK |
| | | DRNNIDTAMMEFITGSARNLRQYVERYGFYPKYDETVTYEKDTRGRERMKKKMTERDDFFYKIGNEKECDGK |
| | | RYTVLSGFGLSFFCAVFLSKTQTRNMLADIRLFETSPYPKELDDIIRDMMSIYRIRAPRGKKKLEAGNTGVTLALD |
| | | ILNELRRCPRELYDVLTTEGRKFFEDETDRTKDAFVPDDALSPYTSADNASETVKRIRWTDRFPQLVMKYVDET |
| | | EFFKNIRFQVRLGKYRFKFYDKLCINGEEDLRGLQKEINGFGRIQEMESARKEKYGDMMQPVESERIRIDDDLEL |
| | | DLLQFAEDSRDSSPYITDSRTAYNINNNRIGLYWNELTVNGRTIPKPGDHLPDLGLTPEGKADVDMPAPMAMLS |
| | | LHELPAMVFYQYLLSQNADPNELKTRSAENLIIGKFNSLRRLFTDISEGRLAPLPEDGLVRVLDDRYGLTLREIPD |
| | | KLTDFLTGKKGADQSKRRRVVAERTLLESLRKAIRRRDRYEEDRKKIGEKDNKYAKKAYSDVRHGSLGRYLSE |
| | | SLIEWQPTLEGGKDKLTGMNFNKLQAELATFNSPEKYTRLRETLSAAGMLSGPIAHPFLRNVTNRKIRNIEELYLI |
| | | YLDQEIGYLKTLLNLGYKGGRRDDRNIDFEASTLKTHVDVRKLPLVKGEKRWEKRTPDYCRELAKRYLEVDGR |
| | | RTSIWLPEGIFSKAITDLLXRHYSGNDGLMDRMRDDSLNNNVAYLINAFFETVLDDHSQPFYMSYDIDKNSGKE |
| | | TPNRFARVYDLFDILNNRKIRNALVKVPMTCNQIAERFTAKNRDGRKRILAEIDDRTAGMTPRDIGSHASLKEAK |
| | | EAMRRRLTHMIGDVKDNERCVRRYKTQDMILFLMASQLLGDAIRMEKIDSLDKFRLSAVCNAGFLNQTMDFEF |
| | | PVQMDGNVIYIRQDNMSLKNYGEFYQLLSDDRLPSLLEKLADGGSDRIDYGNLMGELTSYDLHRSRIFKAVHM |
| | | LERRVTSDRQFRIFLNNPCDRRFYINDDASRMAKRNNFRSLLMLLEQSDTASLTESERNLIISIRNAFSHNHYSIDL |
| | | PRIAPEKRLRKTTLLNKDTRPLGEKKASLTTIATLIAARLEELQKKLESKH |
| 21 | JW13123 | MNFEENGNKYLFGAFLNTAYDNYNEVISHIYDVIRRITSNNPTEQISSSQALVSAEEQTSSDEKDKNKERMSFVC |
| | | TFLITGNATSRKIESMLKRAFPFWDDLIEIQTSMSMQLDKTESIFQVQKNCVEELFTALESWRNYTTHYIHPEAIA |
| | | SPTINLLLKSCFNQGIKEIKKRFNAQDEQLKHLYEAEFSYNRETNKDEVKCYHPANYYKFTDESDLLTDKGLAFF |
| | | TCLFLYKRESYLLLNQLKGFKRADEERYRMTLEAFTVMRFHAPKQLMKLQQQQIDKVSLGISMINELVKCPKEL |
| | | YDCIEDEQKKQFEVYPNAETVPDEQEAIGELIRYKDRYEILMLKALEHSSECKNMGFYLSLGKCYMKCYEKHYI |
| | | DGTTDNRYIVHPLYGFGRLQNASTDHKNHLSSFGEELQTHSQLPVCHFATEDDAADSPTSVPYISESYPSYVLNN |
| | | NNIGIKWLNSSEKTEYPNFNLNEGKVSCPTPDYWLSKYELPAMAFYAYLKEKHKNKLQEYPSIKELIDQSQPENT |
| | | KTSSKTIKSRLEERLKTYKVDAAKKLKRLAEKPDGVQSGRLADELARDILWLQPSSPKEGGKDKLTGANFQALQ |
| | | YALARYGFMRTTLSRIFTTAGLIGSGNAHPFLDKINPQDYSKLTAYYVGYLKQKIVYIDKQLEKLARGKVNIQFH |
| | | PTRKLIKQSQINTIEPEKETVFLPRNIFTEKIEQALCQIQGNFKSNIERAKTSGKGVNASKLIHFYLELYLKDAVTEF |
| | | YNIKRKYSCLCYPLIPSEKYNLKEYQNTEERSERIDRIKDFYNFKEVTKKACKGQELSEDEKKLYKKYIELQKVID |
| | | NEKMIRLRKVQDITLYLWMRLFIEENPERIVFNKNDRPENSLLKLKEMDNTILDHTTARKINIDTPNGNVTIEDEF |
| | | KIKDYGRITTLTTEKLPQSLIKFIGILNKPLDKKDRPSLSYSYIYHETNAFYRYRPEIIKMAQDIENKIINELKIDIQPE |
| | | AEGYYNFRQILEEIKEKSSDINLELLIKIRNAFCHNEYKELDNDQHKYLSSLFKPIADIKQTATLAYLLHMLFETEY |
| | | QKVMILK |
| 22 | JW13124 | MKKKKDSHKADWRDDLISIKHLLVKQEKAKAASAPKKPPAPVALIDLSHMEEQRRELQAEDSSSAKPTKSSAK |
| | | AAGMKSVFLLSDKELLMTSFGRGNDAVREIRVQGDEVQALRDDPAYRIEKADSGFDIHGRTPTKGYTDDPRYA |
| | | AEGPPEHRDLIDCRKALEKMYFGEECEDNIHVQLAYSILDIEKILAVHVNNIVYELNNLLRGKEKYYKDWIGYM |
| | | GKYYTYQEVLRKEFKSKNDEKNLTFVHNMILNVMQDKRRMYFGKILCGPSIAKEDNQEKKKQRYEEEREHAY |
| | | YIFLILGMARQMLAHGDESTRSLLFCFDEEHDCNKTKEVRIKLRQTLDAVYSERVKELNDEFIDTSSKDLVMLCR |
| | | VMHARSPEKKKACVRDYYDFVVRKEYKNQGFSVKRLRETIILRTPQAAVVNDAQYDTVRQKLNRLFDFVIFDH |
| | | YRKHAAEAEQIVASLRAAQSDIDKELIYRHAANNLWQAIGKTVLQDILPMMNGNAIKGIAADPDIKLSMISDIMI |
| | | SDDADYFVKFIYLLTRFQNGKEINDLLTTCINKFENITAFISVMEEQGIFKEFRQEYSMFDNSAAIAKQLREVNSFA |
| | | RMSMKSQDLGLADKLVMFEDAADILGYSDPQAGKQLLDTILNPSQNTGFRNFIASNVVKSNRFHYLACFCNPKT |
| | | VRKLAENRAVIDFVLSEIPDAQILRYYTACTGAKEGADPGEMRAFLAEKLTGITFEEFRNVKQKDRFASPEEKLD |
| | | KVRKQAIIRLYLTALYLLVKNMVYVNSRYFLAFQCLARDNALHGYTDNDIKNDPAAFARRFLSEPRHRKNRRA |
| | | VANIEQNMANSDPWIVREYRNAAMHLSAIRRMPEYIADVKRISSYFELYHYIMQRHLADQHTRKEGEGEEAVK |
| | | NEVVLRYFADVRKYGSYCKDFLWALNVPFAYNLPRYKNLSIENLFDRNRPGTKAGGPTKSAGPTGA |
| 23 | JW13125 | MKIIRSKGQSFTEHSKDESNTDHLERGLEAKRERKLVHSEHKDKTLFHGRELAQYVGEDHALVLRQAISVLDKV |
| | | IRPPRRLRDFELQAYVDRAKLAHELWLLVLNKCKGDFTKHPDSDEVQKIFEKTWSERAHPYTRDRVQPLNPCG |
| | | DENGKYLQSHPKEGVTRQDFEGRWYSALWQENNTTRKPDYATTAQAIIDHLFKQELQFDGQERTKSGHNDDK |
| | | NKNSGTPEPTGKGLVEARGLTIKDSVNDPRMGKQVTKNADLREASKEIDSHYFGKDIAHQICSAFKNRSENDLD |
| | | FNPAGEIGALLYNHLPTALGAANGDEHIKSAIWKRHNQVRQFYQKLLRTNRFKRAFRTRDMDKLDNLLPKDTE |
| | | SLTRVLDAKAENAYLSELIRLGKVIAHAADIPLPPVDGENLQDQFKARLEHFATSAGQSEIKRNETFTRLWRTST |
| | | ALSLRTLTAWVEPILGTNENNNPRNFDITDNNFRGSLNHLLGENTRGDFSWDRIKVIFGQKAYPALCDISDKGRS |
| | | HIFSSGESQNEWQDHQEVVWALVRLATQIRHGTNHFNVRHRLVDEITGGLLTGDNHPQHFHNRPGNIVHSSMLE |
| | | RFDKLLAFDTRLQWQVLLDDLNAFSITKYLNNPQLTTLLQELIKGQTGTDLATPKFMALLRRAANLARAEEKEY |
| | | GEEKMPEESEFLSTNALRRFGPLELTDLSKKKDTSLNDVRIDLFRHLYRTGFPAWLEEKRVDDDYLKNAIRAVF |
| | | DARAFREEAFAEEQRIVAQGTLLESSDVDAISDLSALFATLAANAAGEERLRRNSRARAKTRETVRVQSGGRPD |
| | | TKEKPSWQATLREDIYGHLFAEYLNEEHLSWIWDIKEPHNPIAEDLKLGEIPDLDQSNRENWESQFYTWLYLVPS |
| | | DQVSLLRHQLHKTLALDAAADKSVSGSAADEQDRATLSRMSNLMALYTAVQEAGFDGNEHVEAYGEEADYIF |
| | | YADHDQFKSIHSQDDLTNTVPGTRRGLRQILKFGHLSAVQRIYEKHRVTQAEAEDLIELYGKKRGGEEPQINDFT |
| | | SRSKLHKEIEGLWEKQQPREKHTTDKDIEDKCKAYKTSAIKMALHNFNIASARLTDHADLHHLLIRVVTRLLDFT |
| | | LTWERDRNFLYLGMLCDKLRTIEKDDRGSVLSSTLNLELHHLPYVVKKGKSAEKKRWSICLKIGDRHAQESAEI |
| | | GFSGSDESTIQQALLQEYGLLRLWDDEMGLSLDARDIELDLLPTDHRCLFIKYFVKLAGENEKDVAAREKAPKN |
| | | KPITDHSQRSGAYSYSYKISKKQIRNDFAHFNILTKERLNLTYLTNAVRSLVSYDRKLKNAVSTAIADIVSDAGLE |
| | | IQWTLERDRLKKPLVIPRVVHHLAWLPKINGEDVSFYLPARSARFTSMVKALFDFGTEGNVVEIEKDGTGITGRL |
| | | EYPSALAERYTITKTATDEAGDGGEIPETPAQICLIPSEILTSLENLPQEESKTMIKNRR |
| 24 | JW13126 | MNHTIGKGISYNPFKHQDKHYFGSFFNLADNNIKDVFNEIAQRLNHKDSNPKNIINHYTDKNISLVDFEKFVSLLS |
| | | NYFPIVNEIDQINCDAKGSNGNYKPKTKEERLGYFKKTSYSLLEAIDKLRNYYTHYHHEPIEIDLNLFPFLDHVLL |
| | | KTALNTKKNYLKKGKTKELIKDNLKQQFDFIFNIKVEEYLKEKNPTSKKIERAGGKIENFNKSLVEEITSAIYNDA |
| | | IKDFIYSKGENAELSNRKKTSFSDKDYFYYNDDDHFSLPISSSGIVFLLSCFLNRKEIESLKDNITGYKGKIPNSELT |
| | | LKHNGIRFMTTHRIYSHWCYKGLKTKIRTSEKATRETLLLQMVDELSKVPDVVYQNISDDLKKTFIKDLNEYYID |
| | | NQENQENLYNSKVVHPVIRKRYEDKFNYFAVRFLDEFLDFPTLRFQVHLGNYLEHSTSKNIGNSKTNREIKKKIF |
| | | VFGKLSEVNKLKNDFFEKNKQNDTSWQFFPNPSYHFPMENSNDLKKANKIGIYINHDKSINKFKHQTQKQTHEN |
| | | KIKLIQKVIGDKSKIKIGQPIAYLSMNDIHSILFEALKNPKIKESGKIDGEHIEYKITHQINKQIKEIQNKNENAKIIK |
| | | NQQSKQETDFNIDKLIKDVNKEITISEDKIQTLKENKKQYQNYQRSKKSEKHNNSRKRNHILYNSEKGEIATWLA |
| | | NDIKRFFSKDFKDKWKGHHHSNFQRNLAYYNTQKKEVELLLKGLDYRKTIPMLDFSKNSFLDFYRKYLEKRKQ |
| | | YFKNLLSELKNHKSGKHDSIEKLKKKCFTHFKKRNYRNKTIEDKIQNMLANPIFIKRGFMDTKPTMIPGKNFNQN |
| | | KSDFADWFVAFKEFSEYQKFYDTNLYPIDLSQKSKSATKKINTEIYKQKKNDWAIWQMTKYIFKDIFQQDSQNIS |
| | | LSEIYQSRQEPISNAKNTATDKRKQNSIWDSTIDLQLNRKIKISDVKLKDIGNFRKYENDQRINTFLDYGDFSDW |
| | | MAYLPNNWQENFEYKPINVIDIQLDDYEKIRQNVLLQEVQNLEREIYNNVSNKNEILVQDSKGNKNPNFKAYIL |
| | | KGLLKQIKNLDITNFKLPQNAKEFDTLTFKHLQTLSVIEQKTTLLILLRNKFAHNQLPNKAAYEFCQTILEREKSQ |
| | | TYAHYYSELFKTLKKELTDI* |
| 25 | JW13127 | MENTIEIQKQSIRTLNNAPQYFGLYLNMARLNIFNISNYLAEKFGLAVLDEEEKITISFLGDKGNKEYKTKQSYIY |
| | | FNLIKYMPVAKVFDFEKLPKNEIDKTKNNSGKDFNKLSDTLKVIFKELNEFRNDYSHYFSTEKQDKRKLKISSEL |
| | | KDFLNINFSRALSYTKNRLKDVFSDDDFNIASQTVVVNNDNTITQDGIVFLISMFLDMENAFQFINKIIGLKGTQT |
| | | KAFKAKREVLMTFCVKLPHDKFVSENPQQTFSLELINELNKCPKLLYNVITEEEKQIFKPQIQTEQIENIISNSVPD |
| | | TIDDYENYIESITKKVRYENRFSYFALKYIDLKELFKKWRFQIDLGKVVLDKYSKQLNGKDEVRTVTENAKAFG |
| | | RLQDFYDQEKILNKINIGENKTRFYQFAPHYNFDVQNNKIGISKKETTAKFISKNTTHKVKFNLLQPLPEVFLSIHE |
| | | LPKIILLEYLEQGKTEKLINNFIKLSNSTILNKKFIETIKYKLSDLDVFHKRSMGRKEISAYRKDILKYLIARKEKLN |
| | | QILSEHNLSVKQIPTRILDYWLNIADVQEKTAISDRIKLMKRDCMDRLKAIKKNNAPKIGEMATFLAKDIVDMLIS |
| | | EGKKKKITSFYYDKMQECLVLFADKEKKELFISICSELKLNDADGHPFLKNINLSDIRYTSDLYKIYLQEKADNR |
| | | NQRNRTDTSWLAKNFYTLKNVEVFDKKQQRKIFKLQTIVELPQDKSKIPFTIRQLEKEKSSFNKWLEYITKGKET |
| | | TDKKKPVNLPANLFDDTLKKLLKTELSKNNIQYNKEANYNQLFKIWWKEFRKDNTQNFYTAEREYTFEEEKLN |
| | | FKINTQPKFENYYTRFAENVFNKKQALRNEERKTKRNLPPIQKIHVIKKIRNKIGNTEKEIRMLQEEDRLMLLMFE |
| | | KLTKENLNLKLEKVNELLNERIEIKEKIPGKLSFDDLGEKTITRNIVETRKRKEYSVLQKYIFDRRLPELFEYFNET |
| | | DILIEKIKTELDAYNKAKDIVFDLIFKLEEAIIKKDETGIKELNKDQNGNIQTGNIQHKPYLHWLLKKNMITEKEFT |
| | | FINMVRNTFSHNRFPQKKTMELFIKKWNDNNFAYQILETYKQKTNNIIEKLKDM* |
| 26 | JW13128 | MKKKSNIKIMGVKSVHYFNNDVIIENSLSENVEILFKPNYQKIDELKILNSNTNNQTNNRVEFINDNEKKGFTLKK |
| | | KDSSFEKDKAPKSINSYVNKVKNNSFIHNELFGNKNNNFIPQIYSNFEDIRKIILNYIDHIIYSVNNKIIANKCKIEVD |
| | | RLSFDDIGMLPATKTYEEYGQTPEQRENMTDVVLPAFKEVLKDNNIKTGNYGNIKVDYWNIVKYAYKKSRCQE |
| | | YTIRELSNDSLNYINNNINAIAAYSKKVKDYPLLNELNLDRNKYIIHLERKNENEKTAIKKLNNHFSGEEIFKEIYG |
| | | IEKEFQEKKELYNYLRIISLIRQYCAHFITFDSEEDSIKALINPSNNLIKNFKTSFIKNNEKYLLVLYEYQDFYSELLN |
| | | KKNINKENNIFTEFYNYLIKDEAKNLGISVDKLQKRYVFNLNIEDDTKKAEYLNKMKVLIKFFITLELKYDKDFN |
| | | KNDSSDVNSKFYYIEKIKTAKYANNEDIIKALSDKYSDFSIDDYKDEYDSLYNQSKDKIYDELYEKLNVKIDTDK |
| | | LSDYVIDILGKEENNIIKKFTFDNLKVKYSKIEEEVNEYKKWEKIKNKEKKEIFENQIRDLFPDIFKHINEYNEEYE |
| | | KNFKNKIKLPNISTDSKFFNLIYFVSEFLTTKDANNLFSTILTKLDSISSLLSLLNSYDSNSNIIGNEFIDYKFMLDNK |
| | | EINIENICNFKDDIKILKSLRIKDNTKTSKANINDVDFNNIFNSFNDAGMSLEKFNRKIELTTESQEDDYVANNYKE |
| | | LVKYNALSSYKKSVNELNNKKYNKKTLLDLKDEDLKERINNYLSTLDLFKNYNCDKNYIKLVKEAVNYKKKE |
| | | KPLKQYLRNSIYSSKQYQYIKDYANTRLCKYIIANEKILDYILNDKFDTEGMKKHLYRVYLDYKKEEITKDKIDN |
| | | NYNNVNINELKKALQEINLKTLSDNILKNIPEDRNLKAIIGLYFEVCYIVIKGLYDVNSAYMIQIQEYEKIYKKCFG |
| | | ASNFSFNTELLKKFEEYEKNRNPESKSLKQIKGIIGDGKFGHEYVREETDDDEKKKKALIYIRFRNTVEHSSLLAN |
| | | SDILKNFNEDESFTKNEFKITSMFQLYHILIQRLLKKDSSNNKDKDGNPNPYYPIGSEFEPDKKGYSTKALIIMNLP |
| | | YLYNVARFNNLTIEKYYLRRFEGNVKNKKEPNSSSE* |
| 27 | JW13129 | MGIDYSLTSDCYRGINKSCFAVALNIAYDNCDHKGCRTLLSEVLRSKGGISDEQIKSQVVDGIQKRLKDIRNYFS |
| | | HYYHAEDCLRFGDQDAVKVFLEEIYKNAESKTVGATKESDYKGVVPPLFELHNGTYMITAAGVIFLASFFCHRS |
| | | NVYRMLGAVKGFKHTGKEQLSDGQKRDYGFTRRLLAYYALRDSYSVGAEDKTRCFREILSYLSRVPQLAVDW |
| | | LNEQQLLTPEEKEAFLNQPAEDEGGDISDSSSSDKNKKSKEKRRSLRRDEKFILFAIQFIEGWAAEQGLDVTFARY |
| | | QKTVEKAENKNQDGKQARAVQLKYRNQGLNPDFNNEWMYYIQNEHAIIQIKLNNKKAVAARISENELKYLVL |
| | | LIFEEKGNDAVQKLNCYIYSMSQKIEGEWKHRPEDERWMPSFTKRADRTVTPEAVQSRLSYIRKQLQETIEKIGQ |
| | | EEPRNNKWLIYKGKKISMILKFISDSIRDIQRRPNVKQYHILRDALQRLDFDGFYKELQNYVNDGRIAVSLYDQIK |
| | | GVNDISGLCKKVCELTLERLAGLEAKNGSELRRYIGLEAQEKHPKYGEWNTLQEKAKRFLESQFSIGKNFLRKM |
| | | FYGDCCQKRCFDEEKGYNTQAKERKSLYSIVKEKLKDIKPIHDDRWYLIDRNPKNYDNKHSRIIRQMCNTYIQD |
| | | VLCMKMAMWHYEKLISATEFRNKLEWNCIGQGNMGYERYSLWYKTGCGVVIQFTPADFLRLDIIEKPAMIENI |
| | | CQCFVLGNKKLNSGAEKKITWDKFNKDGIAKYRKRQAEAVRAIFAFEEGLKIQEDKWSHERYFPFCNILDEAVK |
| | | QGKIKDTGKDKEALNRGRNDFFHEEFKSTEDQQAIFQKYFPIVERKDDTKKRRDKKQK* |
| 28 | JW13130 | MENQTQKGKGIYYYYTKNEDKHYFGSFLNLANNNIEQIIEEFRIRLSLKDEKNIKEIINNYFTDKKSYTDWERGIN |
| | | ILKEYLPVIDYLDLAITDKEFEKIDLKQKETAKRKYFRTNFSLLIDTIIDLRNFYTHYFHKPISINPDVAKFLDKNLL |
| | | NVCLDIKKQKMKTDKTKQALKDGLDKELKKLIELKKAELKEKKIKTWNITENVEGAVYNDAFNHMVYKNNAG |
| | | VTILKDYHKSILPDDKIDSELKLNFSISGLVFLLSMFLSKKEIEQFKSNLEGFKGKVIGENGEYEISKFNNSLKYMA |
| | | THWIFSYLTFKGLKQRVKNTFDKETLLMQMIDELNKVPHEVYQTLSKEQQNEFLEDINEYVQDNEENKKSMEN |
| | | SIVVHPVIRKRYDDKFNYFAIRFLDEFANFPTLKFFVTAGNFVHDKREKQIQGSMLTSDRMIKEKINVFGKLTEIA |
| | | KYKSDYFSNENTLETSEWELFPNPSYLLIQNNIPVHIDLIHNTEEAKQCQIAIDRIKCTTNPAKKRNTRKSKEEIIKII |
| | | YQKNKNIKYGDPTALLSSNELPALIYELLVNKKSGKELENIIVEKIVNQYKTIAGFEKGQNLSNSLITKKLKKSEP |
| | | NEDKINAEKIILAINRELEITENKLNIIKNNRAEFRTGAKRKHIFYSKELGQEATWIAYDLKREMPEASRKEWKGF |
| | | HHSELQKFLAFYDRNKNDAKALLNMFWNFDNDQLIGNDLNSAFREFHFDKFYEKYLIKRDEILEGFKSFISNFKD |
| | | EPKLLKKGIKDIYRVFDKRYYIIKSTNAQKEQLLSKPICLPRGIFDNKPTYIEGVKVESNSALFADWYQYTYSDKH |
| | | EFQSFYDMPRDYKEQFEKFELNNIKSIQNKKNLNKSDKFIYFRYKQDLKIKQIKSQDLFIKLMVDELFNVVFKNNI |
| | | ELNLKKLYQTSDERFKNQLIADVQKNREKGDTSDNKMNENFIWNMTIPLSLCNGQIEEPKVKLKDIGKFRKLET |
| | | DDKVIQLLEYDKSKVWKKLEIEDELENMPNSYERIRREKLLKGIQEFEHFLLEKEKFDGINHPKHFEQDLNPNFK |
| | | TYVINGVLRKNSKLNYTEIDKLLDLEHISIKDIETSAKEIHLAYFLIHVRNKFGHNQLPKLEAFELMKKYYKKNNE |
| | | ETYAEYFHKVSSQIVNEFKNSLEKHS* |
| 29 | JW13131 | MNNNILEQSTSGSEKRASTRTLEADPQYFGVYINMARHNVFQIINALTHKFSKQIENAKPINDDENIANDYLINIG |
| | | KGTDKSPTIIEVLNPAHPLHRLYGEHVFRALKRYLPLVTIFEVPSINTEDIIKDTDDILPVLFQDLYEFITLLFYELV |
| | | KFRDAYTHALALDEQGNIVPRKIDLHPNLRHYFEHLFYKAPDYTLRRFRSTQEKIAHGFHEKDFALLKAHNLYQ |
| | | DPHSNELTTQGLFFFTTLFLEQKYAYQFLKRFNGFKNETIQPFKATLRVFTAFTVKLPNDKLASDHPGQSFLLDM |
| | | LNELNKIPQSLFKHLTPKDQEMFERINDEALTNILKNTDEASFKDVDDLLGKMAPLIRREDRFPYFALWFLEQTK |
| | | AFNHIRFQISIGKIAYSPYDKTIIGETSPRIIKFDIHGFGSLAEFSDEDTLLTTLKRQLPAGSPHASLTFSQYAPHYHIK |
| | | NNKIGILVDYPSGDRPLSNITQQPDGKIKVNHIKPTAFISINELPKLVVLALLGTNKDNKPIGNDIERLIRSFITKFKN |
| | | QLFHLAPLTKIKNGLTFSPETFTRRMDKDKMVKGRIPKSELAKREEYKIYLEKRKAVLQGTTDLKLNVNQLPTKI |
| | | QNYLLNIDEVSDEKSIHLRIKTMIKEVEYRLKKIDRNKLPKVGDIATYIARDMLDMVADVRTKSRITSAYYSQIQ |
| | | RALAYFALDKTRLIDLCDELGLFNHQTGHVFLIREIFLNNHHILGVYREYLTKKQNWIDQNLIVKIQERGKRLTK |
| | | YILPTNRPLPYKMKQSINKGFEFNAWLQHKSTMPIDLPTTLFDESLTSLLKKTIAQKLGIASKSEDKFGRLLDRLL |
| | | KQDTQPFYKFERIYRKPFSQEEPEPFNILKLTHTALKQRFGKPAEVNEKIIRHHQRKDQIVRLMCEHLFKNNSELA |
| | | FEGEFLLENLHPASRNNPLDAPMVFEQTIPHTHKKIVAQDSNKWIQEVKKTNNRNKAWYRWTLKDFGTFRKIIH |
| | | DRRLPALVNSITGDEIDFDLVQFELAAYDRARETIFDQLFLLEQFIADKDMSYIKQHKAIERMQDGKGFDEVQFN |
| | | TYADWLSDKGLLTPDEINGLKEIRNRFSHNQWPGISKLGIQPISQEQQSAFVTHLNIKGWVNEHHTPLAMFMEKL |
| | | VINWVSKAIT* |
| 30 | JW13132 | MKIKNENTDKKTELYSIDKYKGRDKWCFAIVLNKAQTNLTENPDLFEQTITKYDRIRKEGWFDEETKKLIYIQEN |
| | | EHKIKGEIKTLAREVLKNLRNYFSHHFYKQDCLIFPKDNIVRIIMGRAYERSEYEIKKNLKEDISIELPALFEPEGKI |
| | | TTAGIVFFTSFFVERRFLHRLMGSVQGFTKTEGEYKITRDVFAKYCLRDSYSVKAQDNDAVMFRDIIGYLSRVPT |
| | | ESFQHIKNPKKQNESQLSERKTDKFISFALKYLKDYGFEDLKGHYTAFFARSEIKKEKEDIEIKDDKKHKPHRMK |
| | | SKIEIHFDKTKEDRFYIERNNVILKIQRKGGRANILRMGIYELKYLVLLCLSGKAREAINRIDDYLNDLRNKIPHIE |
| | | NMNKEGIGEQIRSLPGFVRSQLGFVQIDDEKKKENRLDYVEKKWEKKRAESKELKLNRKGRDILRYINERCKKP |
| | | LTIDRYNRILELLVEKNIEGFYHELEELRKTGRIEKNITQALVGEKNINALHIKICKLVQDELKSLEKEDLKKYIGL |
| | | TPKEEKVVSFEEKLGRILDKPVIYKGFLRYQFFKNDKKSFARLVEEIIKEKTGGLDVPIETEYYSISTLGRFDKANK |
| | | TLYETLAMDRLCMMMARRYFLSLNKILAKRAQNIEWKKESGKEFIVFKFNMPQDTGKSISIRFSPKDYTKLYVK |
| | | NDSEFLARLCQYFFPNEKAIDYHKLYSHGINKYTNFQKEGIEAILELEEKIIKKRKINSPENYLSFEEILNQSIYNDE |
| | | EKNTLIQIRHSLLHYQILFSKNDLTKFYNVMKREGIEKIWSLVI* |
| 31 | JW13133 | MENLSKEQIVETQEQNKRTWGNDPQYFGAYLNMARLNIYNVSNHLAEKFGITAIENEEQISNSFLGDKENKVFK |
| | | NKQNHIFSNLIRYLPIVKVFDFEKLPEEEKISLKDNFGKDFKELSDTLKIIFSEINEFRNDYSHYFSTEKKNKRKTKV |
| | | SDKLKDFLNINYTRAIQYTKNRFINVFSDNDFCLAEEAKLVENDNTITQDGIVFFTSMFLDRENAFQFISKIKGLK |
| | | GTQTKAFKAKREVLMAFCINLPHDKFVSENTEQAFSLELINELNKCPKTLYNVISENEKQHFLPKLEDEQIDNVL |
| | | ENSVPYTIDDYENYIENITKKVRNKNRFSYFALKFIDQKELFKNWKFQIDLGKVVLDEYTKTLQGKDETRTVVE |
| | | NAKAFGKLQHLNDEENVLNKINKSESKTHFEQFAPHYNFDNNKIGISRKEDSAIFISRNTDKKVKFNLKQPLPEAF |
| | | LSIHELPKIILLEYLENGKTEQLINDFIEINNSKILNKKFIESIKSKLTDFNTFNKRSQGRKEKSAYKKEALKYLLDR |
| | | KEKLNQILAEYNLNVKQIPTRILDYWLNVADVQEKTAISDRIKLMKRDCMDRIKAMKKGKAPKIGEMATFIAKD |
| | | IVDMIIGEEKKKKITSFYYDKMQECLALFADKEKKELFIHICNNELKLNESDGHPFLKNIILYDYRYTSDLYRRYL |
| | | EEKADNRNNLNKNDTSWLAKNFYFIEKVEVFDKRLNRMEFKPQTVVKIKDKSKIPFTLRQLEKEKSSFDTWFEN |
| | | ITKGKETTDKKKPVDLPTNLFDNYLKELLIKDLSKNNIKFNPKTNYNELFKLWWKECRKDKTQSFYNAEREYSF |
| | | EDKKLNFKINTKSKFEDYINPEFAEKVFKLKSSRREKENSTGRKLPPLMIKDLEKSMANKIGSIEKDIRILQEDDC |
| | | MMLLMFEKLVDNNLELKLENVDTLMNESIEIKQSILGKLSFDDFGDNVNKNDRIIISKNITETRKRKEYSVLKKYI |
| | | FDRRLPELFEYFETEDITMEKIKLELGTYNKAKDIVFDLIFDLESAIIKKNENRIKELNKDKNGNIQTGNIQHKPYL |
| | | FWLLEKNLITEQEFTFLNMVRNTFSHNQFPQKKTMELFIKNWNKNEYAKQILTVYTEKINNLIEKVIDI* |
| 32 | JW13134 | MAVNYSLREKWYRGVNKCCFTVALNIAVDNCKSKGCETLLKEAEHSKGGITDEQIQQSYTEVEKRLNDIRNYFS |
| | | HFYHGDECLIFKKDDIVKRFMESVFATAVSNVVGGTKESDYKGVVPPLFEQSNEDYMITAAGVIFLASFFCHRSN |
| | | VYRMLGAVKGFKHTGKEELSDGQKRDHGFTHRLLAHYSLRDSYSVKIEETKSFRDLLGYLSRVPQQAVDWLNE |
| | | RNELSEDEKKEFLNQKSSEEESPEQPEPENAEWRTEKTSRRSLRKTEKFILFAAKFIEDRAEKEKQDVTFARYQKT |
| | | VTKEENKNQDGKQARVVRLKYEEDKKDDEKPREHFNLEWMYYIRNEHAIIQIKPKDKEAVAARISENELKYLV |
| | | LLIFEGKGGDAFNKLSDYIFRMTQKIKSGQINPNEARLPSFLKNPVKNITDKMVRNRLDYIRGQIKDVLEKINMEE |
| | | PQNNKWLIYKGKKISLVLKFISDGISDIKKRPNVKEYDTLRDTLQKLDFNRFYERLKSYVSDGRLAAALYDKIKG |
| | | IDDISELCKKVCELMFARLAELEKKGGFELYRYIGMEVQEKDEKYDEWNSPQKKAERFLESQFSIGENFLRESFY |
| | | SEYCQKQECIDKEISLNTSVKNRKSLVYIVKEKLKDIMPLHNDRWYLIDRNPKDFERKDSKVIKGLCNTYVQDV |
| | | LCMKMARWYYGQLNPALKNNIKWDETGQGHGYDRYKLSYRTNFGITIEFKLADFTRLDIIEKSDMIENICRSFIK |
| | | PNRTISWYDFKQDGVEEYRKRQYKAVRAVFAFEESLIIPGRDWLSQGFVPFIKNEEYVKKGFSLFVLDEAVRQL |
| | | KIKGSDKDAMRQVRNDFFHEQFQAKDEQWKVFEGYLSCFMIDRPKGEKNKKRYNGNKK* |
| 33 | JW13135 | MEEKEKISIQTIQDAPYYFSHYANMARHNAFLIVNEINEKVFQRSATKEDELLRESFLVKPPRQQADIIGKIDSLLL |
| | | HHFPFLAYYENKSAGSARVELKKYMDILNQFRNEVTHYKHPVEPKCIVNLQEVFSSAVEKVQERMIAFSEKDFE |
| | | HLKPSSSDYRHYILTEAGINTLTDTGLYFFLCLFLEKKYAYKFLAGIKGFKNNTDHTHKVALEVFTQFCCRLPYS |
| | | KSASSDIALDMLNELNRCPKALYQVIDNEQKELFTVKKEDIVETEQGTEDVPEPVMIRYDDRFPYFALRYFDESE |
| | | ALTDISFQLYLGRKHKKELHRKKLNGEMRTHNLLKEMHVFGKLADYQTEEMTKSFIESKAVEHYAPGYRIVNN |
| | | RIGVLLKAPEKCSLTSSIPQKTNTAPSAILSTHELAALYLYNRLYKQGSADNSPASIIKKYIENFNRFIKDLQSGKS |
| | | QPVESGGFRRMRNSNRDEEERLKLQKAKQELQKQLDVYELKVSYLPDACLEYLLSYKKDNKEFMLKDTFRKM |
| | | WKNADEGLEKIRKKEEQKKEVKQEKLAQELAQDFVYLTPPHKELSENGTEKSVDKKINNMEFDVLKNMLTYFP |
| | | LYKNRLKPYLNILKDTENKWQHPFLHLVLIYDFDSCNSLKDFYVRYYEYKKIWINKFIQQDKRKRWNLVPKQN |
| | | VNDLIKKYDYFLKTTVKFKSAKEKEYSKDAIFLPKGLFDAHIAEAMKKQNYEVKETDNAAYFLKVYFDGKSQN |
| | | YYSLPRYYDASAVGWLTHEARDILKRKIEETWETKNDEEFRNKLKKLSKDIRESETELLHQQTNDRTLFLMVND |
| | | LLSGNTNNYKDIVDLGYNNKSENSILNKLYPIKETIHDKIVTAELPIKRYGEFKRFMKDRRLPDLLGYYGEKEIPL |
| | | KVLETEMETYDRMRKDAIEQIYNFEKLIYDNFESELKKSVDPSALYIDHNHYLNFVKQKFKLETVITEMQSESFR |
| | | ELRNKIEHNQFPPREDWIIDKISQDTTTTIITEKIMNLISTEYKKIWREVESTTSISDME* |
| 34 | JW13136 | MKSPSKNPRSEQAVDYDFFVGGRSKSADLTIYYNVAFDSVVKIKKQISNWPDSRQYAFLAEYLWKAHYLQKNS |
| | | NGYEVQPIDEWIIQALIAKVEAIRNFHSHIWHDHEVLIFEKELKDFVEQKYDEAKAALSAEFPGAMLEYEDLART |
| | | GRMKKFSLFKRVPERKKDFITVEGRIFFLSFFLSTGEMNQLLQQRRGFKRSDMPLFKMKRLLYTFYCHRDGASM |
| | | LDFNHKEQFIDSMDEQQKRDIFKARTAYKLISYLIDYPWYWGSRDSMPLFDEKGEIIRNVGQLKDYLEKNPHLLP |
| | | ELNFHLLDRKIPATGKFNTRKEKENFAKLQDDRYRLGTIAFNYLPIPNYTFHIDFETLHRLVLLQILHHGSKKEVS |
| | | PYQVLELNLRSIVENRVSLYNILCKQDNERTGEDLDFLLDRKNQRLRGGRKLTEFGISYFESIEKGSGAKIRDSIQL |
| | | ANYMRSMDTAWLELPYEGAHKVEIQPLDPEPIMVYQQDFLEGTTQKFRAGNRFMFYVSRYLMDFAGDNWYW |
| | | GMERFEMRKKDQNARSESLIRTKEYLRASDIPLNGDYRLTLDNDHIFLAMPKNGPGKKNHEKFYQFALGPRAM |
| | | RYLGAYIINNRADFVSKAKGFLDTLVRDLDRLHKNEYWNMEHGYELLESPFVSGFLKNVPTNIEKLLTLVKTRIE |
| | | YIKQEWQQALENKPYLSRAQKNKLIMDAYRLLQWPKGNDGNPRFFRAHEYNQMSVCHYSLHLKDNEQDKESR |
| | | KQIKKRFKSKYDHLFEDVFKLNTRKPPIPRSILSLLDEADSLDELMKKTFEECHQLLDNRYSQLKSLPNQQLKREL |
| | | PELCRLLGISVPLNILREQEKKALKEKHNTTLCVKPFDIHPMLVVKYFFPEQYKASKSVEFVNKNGEKVKKQPTL |
| | | TIFSALRKNRALREKLVEDFYDEQNSAVLYPDDDTKKQKEVLTGLMNTTCTEDILLWWMANEYLVNNPYTQA |
| | | MGELLMKKPRSGRQYIGDLHKLTILQPLPKESGKRQIYMSILMHQLDDLMFIVQKSRLGKAAEHFVQRCSTENGI |
| | | WAPEIEKLSTNQLAGAPFPDGTINNPIPFQLLLDEIELVRRTGLELGRYLLEFEKKILQQALNGYESKEAFHKWLK |
| | | NEYEKTEYYGKYPAYFFNFDSILKLSGTVDNNIDKDLNITLSDYRNITFHNDIPSNGSFSWLTREGSSVRQLLGIE |
| | | KNLHEKRDRSEYENNREE* |
| 35 | JW13137 | MEKEQGLYSIDRYQGAGKWCFAIGANRAWDNYNERPKLFSESLLRYEKATRRDWFDEETRGLIKKSDVRQRLR |
| | | KIRCYFSHYCHDNTCLGFDPDDDLRKIMEKAYERAIFEQRKHLSTETDIETPALFEPHGRITAAGVVFFCSFFVER |
| | | RILNRLMGRIPGFKKTEGEYGATRQMFSKYCLRDSYSIRASDSNAVLFRDILGYLSRAPSQYYRHNKDQCDKDG |
| | | HPERKKDKFINLALRYLESFVPARLRNHTLSVGRKEVVRMETNAVAEGEGEYRPYPPKAKVKVVFTEDDPERPY |
| | | YITHNTVILQTAKKEEDIHHCKVGVNELKYLVLLCLQGKAEKAVAGIEGYVRRIQGRFADHTNKVARDDDERL |
| | | VRGLPEFVRVASGIETPDEVRELKSRLDHIRKKWQTKKAESAEAQLHRKARDVLRHINWESQRPLGIEQYNRLL |
| | | ELLVNRDLESFAAEMKELKRRGLISEELLKSVEGIRNLNTLHVKVCNLVLTRLEHLVENDPEELKRHIGIVPREEK |
| | | EGPSYEEKVRAFVQQPMMYRGFLRNSFFKGSGKSFAKLVEEELHKKGCPDVPLGTDYYLVRDLERDERKNRFH |
| | | NDNAALYETLALDRLCVLMARDCLVRLNRNLEKHATRISWEATDAGDTICLELPRRDRDHESFRLSFGVRDYP |
| | | KLYVMDDPVFLCGLMKHFFPDNQAIQYHELYSEGINKYTAMQAEGIAATLKLEEKTIKEKNMQIPATGYIRFCEI |
| | | VSQSDFAPGEKRVLKNVRNGLLHYHLEFEPTEWAEFREIMKREGFDTAKKRKSTRKK* |
| 36 | JW13138 | METQENELFENRRRTLADDPQYFGAYLNLARLNIFSINNYLAKEFQQTELRDDELIQNGFICIPSQKNRNDNKLFE |
| | | RLIRLMPVIKQFDAARLPNEEQKSGQAEGKDIKTMAKTLKALFKDMQEFRNDYTHFYATDKQECRKLTVSSET |
| | | ATFLKASFARAIEYSKVRFKDVLAEDDYSLVAGKELVEDENTITTEGLVFFTALFLDRENAFQFIGKVQGLKGTQ |
| | | FKSFIATREVLMAYCVRLPHDKLISDNPIQALLFDILNELNKCPKKLYNVLSEEVKKEFRPELSESGIENMLANSF |
| | | NDEEREKLLDEIEYKEYIEQLTIRVRHSNRFHYFAMRFIDEQELWPGIRFQIDLGKYTLTQYDKQVLGESIPRSVV |
| | | ENTRAFGRLSNFLTADPDSLEQRIDVDHRANGFEQFAPHYNADINKIGIAFTEKEQLPYVVPTKNAETNRVKYSF |
| | | KQPQPDAFISLHELAKAILLEQLQPGESEKLIRAFCSTAENKLFNLEFIESIKWKLPDWKPFQKQCDTKKSRAYITK |
| | | KKMFAYAPRDRKKLLDELLAPYGLNWKQVPERILNYWLNIEEVHDARSIKERILLMKRDGLKRLKQLEKHRKD |
| | | PSIRIPKVGEMATFLARDIVDMMIDKTKKQKITSIYYDKMQECLAFYADAEKKRQFIGLVRELNLNAPDGHPFLK |
| | | NLNLESIRKTSEFYKLYLMEKVEKMIENGYKSNGDKRFKNIAWIIRVFEEKQKNEKNGKDDTIYRIPNNRNDIPY |
| | | TIRQWEKETSTLEEWLHTINHGKQEGDRKRPIDLPTDLFDARLCELMKGQMQENGQTFADTAKYNELLKQWW |
| | | SLREDETQPFYNGARHYVIKEEAVCFMPGAQPRLTSCYADAVEEAYNRLSAERNIARRHNKQLPLIQKQKVEKV |
| | | FKHTLYDTEREIRLLGEQDRILLLMAEQLTPGRETLKLKTVSEELTRPREIKKEFPYISNYNAQGEKLPKDEPKPKI |
| | | KVTVTGIMQLKNSNDLNRFAHDRRMPELVAYLPNATIDADTLRYELMDYNKARLTVFDAVFRLEKAIIDQDRE |
| | | GVITLFTDEQGEAKQGRMQHKPYLQWLLNKGIITQPESSYLNMLRNSFSHNQVPHIAVALNGTEVVPVKNIASTI |
| | | AGAYKQKIETIIGKIG* |
| 37 | JW13139 | MKKELNREDVFTGGKTPELTIYYNIAYFRMAGLINALCGNKLERDKDALEQFMKAFINGKQKLTDMQFVKLCD |
| | | YLWKGYKYDKNRSEYSLTENDKTMVLKMVAKLQDIRNFQSHIWHDNKVLVFDADLVQFIKNKHLEAIDAQRQ |
| | | RFSKETEVYEKENRELKKKKNKELLFDIHDGLGYITGEGRNFFLSFFLTRGEMTRFLKQRKGCKRDDTPEYKIKH |
| | | LVYRHFTHRDGATRTHYGYEDNMLDQLPDKQEFLTTRHTYRLINYLNDIPEEVTNPELFPLFNTISNGILIKESVG |
| | | TYIAFINKMPLLSDFEFSEVWGKDGKPYTNLVEFYHKTQPGFKFRTDLNSFHKIILNLIRDENYTGFFTTQLNLFM |
| | | AHREELILVISKPLITPNDLLLIEEHYRYKLKANDFVREKLGEWKEKIEKGKWEKANEQKDKLINLLGGVIEITFY |
| | | DFYWQKDEKPRNENRFMEFAIRFMADFNLLPDCEWEVEPLQIENDLIANRTATPDLKKSGSQFMNQLTDNYRL |
| | | KFNDGQIAFRYQNQVFIMGHKAVKNLLIACFDGKEKMLNGFMPALLADLKKITNEHILKNHQALNTLSLLNNDS |
| | | IPSYISRQWGEAEPITDMKKKAIARMDYITQQFEALIENHHYLNRADKNRQIMRCYKFFEWQYPQNSQFKFLRR |
| | | NEYHRMSIYHYCLDKEQHKYDKKGHNYLYNRLIKESHNESGNIEQHLPYQIRTMLNDAKDFNDYFLRILNATH |
| | | KILTDWKNQLKQGREPNNYYLSRLGFTGGLTQKVVHTRLLPFSIHPGIPVSFFYRTEMNQNPSFNLSAKVWNSES |
| | | PFRVGLKESNYQYAKYLGLFNEIKTQRKIIGKMNQLIAEDALLWQIAKKYLDKCHPKFSEIINQQVNGKQPLNVK |
| | | AIHDTELTIDFPVDKNLYQVVVKMKQLDDFMFIDSKPVLEKTIRYMIKNSLRDKEYQQRVFIETNPGVFKIPYNE |
| | | VVEAMRTINHQALTWARLLLDWESKTVKTIPEADKKAKLQEAYNKRQPAFFNFNDICRYADDGKQLKDLRNK |
| | | TFHTEVPETWTYDEMRENKTIKMVLGINETTFLKKDYAIENARTIE* |
| 38 | JW13140 | MRKNYQTKPKAPISTDQLFTGAKTPEYAVFYNIAYWWMSEMLKRLYKNTDTSIHEDTSPFIQRFVNDGEEINSSI |
| | | FSDADYIKLRDYLWKGYLFDKNKSGSIITFKDKVIIHKIIAKLAEIRNFHSHYWHDNSILKCDEDLKSWIDFLHNY |
| | | AVDKLRVEYSKEIERYESNRDNLFDMHGSDYFITQEGRNFFLSFFLKKSEMSRFMQQRKGCKRNDKPEFMIKHQ |
| | | VYRYYTHRDGASRNRYSIEETVFSALDPDSKSEINKARQVYKMLAYLNDIPSEVIDKKLFPLLLNDEIVEDSTQII |
| | | KFIKKHNLFSNCKFGIIEDSKGEVIQKEVLFSYVDSKYNFRIRIAALHKLILDIIRNSKREEEFYNTLKTFGDFRDQF |
| | | PIILDDIKKGIGNIEELNEYYIFKLKADDKTRGLLGKVIDDYNKNQFIKLKDYNELKEKSVDEPIQLIYHNFYFESG |
| | | QKPRSGNQFMEFAVKYLMDKNLVPNWEWLLEKYESEEVEKKLENNNPKKVVKHIKAYFSYIPDGYRLHFKED |
| | | HVLLRLKYESEIIIEVGENAMKNLLITHFHGKPINEVVEKTVSDINKIKHVAKNNKTLTEKLELLNDKTIPRYLKL |
| | | MMHDESVMQNETINYSIIQAKNRIDRLIEYFEDLHSDKVKLNRREINIQLIRCYKYFDWNYPNNSEYKFLRQNEY |
| | | QLMSIYHYTLAKPKNAKHHLLILRKELFGDINKHLPTNLKLILEEATSIDELFKTVLLETISRLNKWKNRFSSMNS |
| | | NQKKDCLSKLGIHIPKIPVPDASNKANLGQMKNLPFMLHPSLVFMTFKYSRSSTIFSEYRDNPKNSKGLLMDHYN |
| | | FQSYLDLHSVLIDQKQAHKIVGTINNLKTLDTLIWQMTKKYLDDTTSDILKNITNELINSSTENFNVNQLHNFEFPI |
| | | FKSDKSEVKPFKVIVKFKQLADFMLITEKPLKNVALQLFKRYQTNEELEKLEVYFKNGFYEIPYVQIKQEMDRIY |
| | | RESLILADFILDWEKSIVDKIPEIEKQKLIESAKQNTNGYKAYINFDTVCNYAGLIKEEQSELQNLRNHALHAKIPD |
| | | KDWTYKQKLNDNRIQKMLYITLEKLEKYEKKNAYYESKVLHNTY* |
| 39 | JW13141 | MEKSKLDNEMTFAEDAYYFAHYANMARHNAYLILNHINKSIFGNESTLKEENLQIAIIKILDPKNGADHHAKAIK |
| | | LLNHHFPFIACYYKEDIKKAVENSAYKEMNDKKLDHLKEPLTLKQILKTLTGIRNELNHYGYSRKEHPIPFFQDL |
| | | FNDSIQLAKKRMIEFNETRFNSIQDSPYHKMVDPKDSVLTDTGKLFFLCLFLDKKNAFEMLAKVKGFKNRTDEK |
| | | YRATLECYTQLCCKTPYPKLESSDISLDILNELNRCPNALYQVLSEEDKLKFKKEPNEETAEELNPIMKRYDDRS |
| | | AYLALRYFDRTKCLDNITFHLQLGRKVMGEPHKKVFDGKERTHQILKEMRVHDKLSTYETEEAQTFFQSKIESN |
| | | EVELFAPHYRMVGNRIGLSFGTPSEKKEGYSIPINNQPAPQAILSTSELGALYIYNRLYKENKITQSPKNLMSTYL |
| | | QTMNRFIKDWKNDGIKPETSSNKIIKERNQERTKQTINELKRRTDHLQKLLDEKYAGLKVKHLPDICREHLLNYQ |
| | | SDDNIQQAIDFLKKTLSDTNFKKKQLEQFERNMVYRSSNSKQLKIGEIAQELARDIVFFLKPIKKEITNRNKEKVT |
| | | VFQKINNQDFDILQNRLAYFSLYKEELCSFFDDLLKKKHMKHPFLENVKAETKWSNGLIEFYLKYLEHKLKWLN |
| | | TYTKNAPNNTTRPNYTTSNRINKNRSTPNKKDEIEEKEIDPEKIKETFIEKYNYFLKVKTSSPKNYQGKAIFIPVGL |
| | | FNDAITKAMKESGYQVEDDEKTPNAAYWIKSYFHGATQPFYQLPRYYNRAKKDEERDLVEAALLKKEIKNYYV |
| | | KANKKTQNDFKQLAKRIRKQEEEILTQQTSDRVLFMMATDLLNGGSDGRQTIECEQINTLGFNLGENKILDENR |
| | | KVSMNIYGRKVVSELPIKRYGEFRRFMKDRRLENLINYFPEGKDIELGVLKSGEVEAVGERFSSSTLANELEFFDS |
| | | QRWLLMKSIYEFEKKIYTHFFDVLTKNVPEKEMMENNYIAHYHYIECAKKLELSVSELEKDDFKELRNKIQHNEI |
| | | PYSQWIKNRISTKDGHLITKQIVELVLSIYQQMNNELDQLIRK* |
| 40 | JW13142 | MKTNPLIASSGEKPNYKKFNTESDKSFKKIFQNKGSIAPIAEKACKNFEIKSKSPVNRDGRLHYFSVGHAFKNIDS |
| | | KNVFRYELDESQMDMKPTQFLALQKEFFDFQGALNGLLKHIRNVNSHYVHTFEKLEIQSINQKLITFLIEAFELA |
| | | VIHSYLNEEELSYEAYKDDPQSGQKLVQFLCDKFYPNKEHEVEERKTILAKNKRQALEHLLFIEVTSDIDWKLFE |
| | | KHKVFTISNGKYLSFHACLFLLSLFLYKSEANQLISKIKGFKRNDDNQYRSKRQIFTFFSKKFTSQDVNSEEQHLV |
| | | KFRDVIQYLNHYPSAWNKHLELKSGYPQMTDKLMRYIVEAEIYRSFPDQTDNHRFLLFAIREFFGQSCLDTWTG |
| | | NTPINFSNQEQKGFSYEINTSAEIKDIETKLKALVLKGPLNFKEKKEQNRLEKDLRREKKEQPTNRVKEKLLTRIQ |
| | | HNMLYVSYGRNQDRFMDFAARFLAETDYFGKDAKFKMYQFYTSDEQRDHLKEQKKELPKKEFEKLKYHQSKL |
| | | VDYFTYAEQQARYPDWDTPFVVENNAIQIKVTLFNGAKKIVSVQRNLMLYLLEDALYSEKRENAGKGLISGYF |
| | | VHHQKELKDQLDILEKETEISREQKREFKKLLPKRLLHRYSPAQINDTTEWNPMEVILEEAKAQEQRYQLLLEKA |
| | | ILHQTEEDFLKRNKGKQFKLRFVRKAWHLMYLKELYMNKVAEHGHHKSFHITKEEFNDFCRWMFAFDEVPKY |
| | | KEYLCDYFSQKGFFNNAEFKDLIESSTSLNDLYEKTKQRFEGWSKDLTKQSDENKYLLANYESMLKDDMLYVN |
| | | ISHFISYLESKGKINRNAHGHIAYKALNNVPHLIEEYYYKDRLAPEEYKSHGKLYNKLKTVKLEDALLYEMAMH |
| | | YLSLEPALVPKVKTKVKDILSSNIAFDIKDAAGHHLYHLLIPFHKIDSFVALINHQSQQEKDPDKTSFLAKIQPYLE |
| | | KVKNSKDLKAVYHYYKDTPHTLRYEDLNMIHSHIVSQSVQFTKVALKLEEYFIAKKSITLQIARQISYSEIADLSN |
| | | YFTDEVRNTAFHFDVPETAYSMILQGIESEFLDREIKPQKPKSLSELSTQQVSVCTAFLETLHNNLFDRKDDKKER |
| | | LSKARERYFEQIN* |
| 41 | JW13143 | MPXXNNQKKKADSIKRXSPSPSRINGVAKPRPLVVDENNKHVLGGYFGLGLNNFYKATLSIFARTGIKIASKNGT |
| | | VLYPEEKIGQVIRVLRRSISDTSGNLTHEEKVWSNAFKLSSSQEMRLQKLIFHHFPILGPIMADEGAYKASTNKKS |
| | | KVTDAYSMTYGVTLEECLDVLGDLAQTLVDCRNSYTHFYPYNSKESTADQLALQSKTARYLDKVFTASRRLDK |
| | | DRNNIDTAMMEFITGSARNLRQYVERYGFYPKYDETVTYEKDTRGRERMKKKMTERDDFFYKIGNEKECDGK |
| | | RYTVLSGFGLSFFCAVFLSKTQTRNMLADIRLFETSPYPKELDDIIRDMMSIYRIRAPRGKKKLEAGNTGVTLALD |
| | | ILNELRRCPRELYDVLTTEGRKFFEDETDRTKDAFVPDDALSPYTSADNASETVKRIRWTDRFPQLVMKYVDET |
| | | EFFKNIRFQVRLGKYRFKFYDKLCINGEEDLRGLQKEINGFGRIQEMESARKEKYGDMMQPVESERIRIDDDLEL |
| | | DLLQFAEDSRDSSPYITDSRTAYNINNNRIGLYWNELTVNGRTIPKPGDHLPDLGLTPEGKADVDMPAPMAMLS |
| | | LHELPAMVFYQYLLSQNADPNELKTRSAENLIIGKFNSLRRLFTDISEGRLAPLPEDGLVRVLDDRYGLTLREIPD |
| | | KLTDFLTGKKGADQSKRRRVVAERTLLESLRKAIRRRDRYEEDRKKIGEKDNKYAKKAYSDVRHGSLGRYLSE |
| | | SLIEWQPTLEGGKDKLTGMNFNKLQAELATFNSPEKYTRLRETLSAAGMLSGPIAHPFLRNVTNRKIRNIEELYLI |
| | | YLDQEIGYLKTLLNLGYKGGRRDDRNIDFEASTLKTHVDVRKLPLVKGEKRWEKRTPDYCRELAKRYLEVDGR |
| | | RTSIWLPEGIFSKAITDLLXRHYSGNDGLMDRMRDDSLNNNVAYLINAFFETVLDDHSQPFYMSYDIDKNSGKE |
| | | TPNRFARVYDLFDILNNRKIRNALVKVPMTCNQIAERFTAKNRDGRKRILAEIDDRTAGMTPRDIGSHASLKEAK |
| | | EAMRRRLTHMIGDVKDNERCVRRYKTQDMILFLMASQLLGDAIRMEKIDSLDKFRLSAVCNAGFLNQTMDFEF |
| | | PVQMDGNVIYIRQDNMSLKNYGEFYQLLSDDRLPSLLEKLADGGSDRIDYGNLMGELTSYDLHRSRIFKAVHM |
| | | LERRVTSDRQFRIFLNNPCDRRFYINDDASRMAKRNNFRSLLMLLEQSDTASLTESERNLIISIRNAFSHNHYSIDL |
| | | PRIAPEKRLRKTTLLNKDTRPLGEKKASLTTIATLIAARLEELQKKLESKH* |
| 42 | JW13144 | MKTNQPKTIYYNWKDKADFAYFAFYTSQALNNTSIILKNISEIIENKVDKTNDDNQLFSNAQVIKILESNNSVAQ |
| | | KSVINLLDSNLPFSVKSISDPFLVKNRLSYFLWLLKNFRNEYAHYHERELDSRTTLDREEDFKKRENLNKRFSKY |
| | | DFADELLKLKKSAVEELEKRLKINNKELESDSVYKSFKNRFLKISSRQNFTEEDFIFFLCLFLSTKETMQLLNGIKG |
| | | KKNTTTEEFQWIRRVFTIFNAKRFQNKIKSDNPKEAFILNIVNDLAKIPIHLKKYLTEEAKEKLIYTVQETEDEEGN |
| | | ILEQKSEAVTKHDKMFSYRCLQYLELFGFAKNINFNINLGKVFINKPYKKTIINDEYDRFLDKEIHTFGKLKDFDD |
| | | SYFEGYIERKETENGVVTTFKQPLKFYSPKYHFSNNRIGIKLYNKNLKLELKEYDEDGKFVVINNQPDYFLSENAI |
| | | PYFTYCMINFGEDKTLGVIKNFETNFKRFLNDVSIGKSIKVQEIEQNYSLKIGWIPSLIRDNLFHDNDKTFEDVVKE |
| | | KINSLKEETQKLIENNNKKPEDRDRNIKFSFKKGDLATFVAKDIIYFMELKEEIVNGKKVVSKLSSIEYDVLQSKL |
| | | AFYGKHEEDLKLLFKKWNLNERHPFLKDVSMEKVEDRRGFKRQIGIKKFFKNYIYQRKYWLNDLKLEINKENY |
| | | HFVDDYKVCKNDTQIKAYASNLLNHTIYLPNDLFSDLILENSNFAVEKANTNFLISKNLEYFGNQWFYNKENYQ |
| | | QGLDTYQSHLRDKQIRKAITLDRLYWNMLKINTQIPEFSQIFEQNNLAAYNSEQTLLEKQIRMSSNFKLAKEDFR |
| | | YLDFDRDFEIKIEGYRKIKDFGIFRHLIKDRRVPSILAFYTKYKKLGSINEEIIRNEILDFEYYKISILKRVLEIDKQIY |
| | | NNLIGKNISIHEKFSENVNKLYLENQKLANKIIEIRNKLLHNKVPEINLENIQKTFSETLFLEMEKSCNELERLI* |
| 43 | JW13145 | MNFEENGNKYLFGAFLNTAYDNYNEVISHIYDVIRRITSNNPTEQISSSQALVSAEEQTSSDEKDKNKERMSFVC |
| | | TFLITGNATSRKIESMLKRAFPFWDDLIEIQTSMSMQLDKTESIFQVQKNCVEELFTALESWRNYTTHYIHPEAIA |
| | | SPTINLLLKSCFNQGIKEIKKRFNAQDEQLKHLYEAEFSYNRETNKDEVKCYHPANYYKFTDESDLLTDKGLAFF |
| | | TCLFLYKRESYLLLNQLKGFKRADEERYRMTLEAFTVMRFHAPKQLMKLQQQQIDKVSLGISMINELVKCPKEL |
| | | YDCIEDEQKKQFEVYPNAETVPDEQEAIGELIRYKDRYEILMLKALEHSSECKNMGFYLSLGKCYMKCYEKHYI |
| | | DGTTDNRYIVHPLYGFGRLQNASTDHKNHLSSFGEELQTHSQLPVCHFATEDDAADSPTSVPYISESYPSYVLNN |
| | | NNIGIKWLNSSEKTEYPNFNLNEGKVSCPTPDYWLSKYELPAMAFYAYLKEKHKNKLQEYPSIKELIDQSQPENT |
| | | KTSSKTIKSRLEERLKTYKVDAAKKLKRLAEKPDGVQSGRLADELARDILWLQPSSPKEGGKDKLTGANFQALQ |
| | | YALARYGFMRTTLSRIFTTAGLIGSGNAHPFLDKINPQDYSKLTAYYVGYLKQKIVYIDKQLEKLARGKVNIQFH |
| | | PTRKLIKQSQINTIEPEKETVFLPRNIFTEKIEQALCQIQGNFKSNIERAKTSGKGVNASKLIHFYLELYLKDAVTEF |
| | | YNIKRKYSCLCYPLIPSEKYNLKEYQNTEERSERIDRIKDFYNFKEVTKKACKGQELSEDEKKLYKKYIELQKVID |
| | | NEKMIRLRKVQDITLYLWMRLFIEENPERIVFNKNDRPENSLLKLKEMDNTILDHTTARKINIDTPNGNVTIEDEF |
| | | KIKDYGRITTLTTEKLPQSLIKFIGILNKPLDKKDRPSLSYSYIYHETNAFYRYRPEIIKMAQDIENKIINELKIDIQPE |
| | | AEGYYNFRQILEEIKEKSSDINLELLIKIRNAFCHNEYKELDNDQHKYLSSLFKPIADIKQTATLAYLLHMLFETEY |
| | | QKVMILK* |
| 44 | JW13146 | MSIMNIITVLNHIHQIAGLKRNELGDDDNIYSHPVLKYIAPENNSTTLPEKRDLIKELLLEYLPFLEAMKDCYKEV |
| | | KVEKDKRNNKYVKDKKQVFIRNYSDIYCIYWTLYNIFSTLINYRDKSCHYRFIPSKDQEEYSKRTEYDTVRIINK |
| | | YYTYALRKTKEKYGYTTEQLSFIQNNRYTDDKKVNFNFFLSMQSSNNNLSHVGVAQLICLFLQKKDVDNFLEN |
| | | LPIYDNRYCLGIDEHMIIKRSLALHSIKLPKERLNSEKKDMAIAMDMLNELKRCPKELFNTLPFDKQERFRIISSDH |
| | | KEVLLMRSTDRFVPLILQYIDCNKKFKDIRFHVNMGKLRYLFSPNKLCIDNQERVRMLEHLLNGYGRIDEVEYL |
| | | RKAPNGTFGNTNIPIKGFGEIERDSADSNNYPYIVDTHTHYILNNNKVEFCFCEDSKNKNVFPTIEKEINGKWYVE |
| | | KTAPHCRISFFEIPAMAFHMFLCGSEKTEKLIKDVYNNYISLFNALKNGALTEENYKDFNIPECDMPKKILDFLKG |
| | | KAEGKSIDGYVKNILKKELKDTRKRIEQAPSEKDQMPSKRNKIGTKSYKRVLTGKLADFLAKDIVKYQPTLCAG |
| | | DDYGIDRMTGMNYRIMQATIATYNDDDDFDTFYNVFKMAGLVDGDERKNHPFLKKVLLSKPQNTVAFNIKYL |
| | | KEKEIYLRNIEKKNIIPPFVNRERNKWCERDCEYYRILGENLLDLPIELPRQMFYKDIRAQLEPLMPEIDFSQTNTT |
| | | YLIAEYLKRVRGDDFQEFYSWERNYRYIDFLKCIRDDNGRICAQYTTTEERESLWEDSKNRTAEYKRSEEQKNE |
| | | KKDKNFEDSLEERISSSHIKFQNTEKHIRRYKVQDALLFMIAEKQFGKYLDDIKAKNFKLKDIMPDTDKGILSET |
| | | MPIDFTFTKKNIKYNIHAKNLKFKEYGDLFILANDKRLESLWKILATDTVNKEEISEEFAKYDFCRPDITKLVLDF |
| | | EKDVLEKYPEIIEEAEKEYKKFDFKFILDWLINKKKLNYGIRNKLLLIRNAFNHNSYPDKKYAEVRELPEIATNLK |
| | | KTFDENAKKYE* |
| 45 | JW13147 | MFDNEQKNLEKEPYWGVFLNQARLNAYIALRDISERLEENAADEDSLSEWPVLKYLDNDTDAVKSRRIFDLVE |
| | | KHFSMLKIIYGGEKEGDLVKRSKEYKIILKCLFRALNFYRNKFCHMYSGNRARKYNEKELIKYLEDCFDASVRKI |
| | | KELRRLDEKDVLHLRRKIAEGKDANKRVIDNPQFRYPFKNEKGELNEKGLYFLASIFLDKKEAHEFLKKQEYFK |
| | | NDSEPKYRATLESFYHYRIKLPRPVIESDVDKNGLALDMLNELKKCPKELFDLLSKEQQEKFRVVDSEDADEEG |
| | | NEILMRRYSDRFPYLALRYCDENQVFERIRFQIDLGRYYFKFYPKETIDGKTQQRSLDKRLKIFGRIKDVKSKVEQ |
| | | EWSGIIKSPDTIEENPNEPYKLKTTPRYNIVDNQIGFVITGDKNLPDVKRPDGRIELEKPDGWLSIYELPGMLFHGL |
| | | KYGFDKTERMIKIYIEKQRKICKEICEKGTITPDDGESMPEALKGGAKAAKRNYSEKKLERMLQDTEQRIRAIQT |
| | | TQKRMDEPGNKPGKKKFFDIRAGKLADFLARDIMALQRFDPAKHGKDKLTAINFQVLQATLAFYGAKKDVIED |
| | | MFKGIGLLEGDNPHPFLNQIDPAQYNSIAGFYQAYLQKKRSYLEDYRKEEEYDEQFLRPKRQRYAQEKREIKTV |
| | | ARQLLDNPVNVPKNFFKKEIEEFVFSQDPSLKKSKMNTAYMIQALFEKHYGRQQPFYSYNRTYPVVSKAIEYGK |
| | | KGKNKKIAKVLMAIEPKLNYMEIKKIVNEMPDGQYEPENLKRNLYEGYKDYEKDERIIRRCKVQDVVSFMMVE |
| | | ETLKDQLDFNGNVLTLEKITPWEASPFKKPVLCHTIISIPFNTKGGHTDKDYVDFIKNNFEGSYDCEPNKIILKYK |
| | | VTSKDTKLKDIGKYRMYSHDRRLPGLLIWKYRPNDQNGNEIKFTEIEQEIKAFERRRIEIAQCLYTLEKKVIDSWF |
| | | TQDELGEEHIPFNKVIDVIKAKMPNFEDKCNVLLKIRNAINHNQFPVYEQAIQTAPGKEIAGKMLRITESYIEQIM |
| | | AKIDPDFGRTEDAESSR* |
| 46 | JW13148 | MIKKPANRFALPKVIISEVDSENILEFKVKYEKLARLDRFEVEAMHYDEKNEIIFDEVKVNDGLIEIVYKENNKKL |
| | | VVEIEGKAYEISGQKIGGKQRLLENRVSKTKVQLELMNHIEDGEGNKRVSRTERELIVLGDIKLYSQIVGREVKT |
| | | TKEIYLIKRFLEYRSDLLFYYGFVDNFFKVVGNEKELWKIDFNTSHSEQLIEYIKYTFNDNLKNDRQYLNEYVTN |
| | | DVQIQKDFNKVKNVFSELRHALLHFNYDFFQKLFNGQDVGFDLDIEFLNLFIDNIDKLNIETKKEFIGDEKIRLFG |
| | | ENLSLAKVYELYSHICVNRVGFNKFINSMLIKDGIENEALKDKFSTKFTGNAYYIDIHNSREYKDLYKKHKRLVI |
| | | NVSTLKDGRAIRQENKKIAELKKQMTTMTTTNSLARLECKLRLAFGFLYNEYNDYKAFKNSFDSNLKNNQFDV |
| | | SNVDTSKAYFLSTYENRKPRTRERIDKVLKEIESLELKGLIANDPLLKFILLMFVFMPQALKGEFLGFVKKYYHD |
| | | VHSIEKDTKDKEEDLIEFMPLSLKLKILGRNIRTLTLFKYALGSPINYNSTDEVFYVEGNRYGKVYKQLGISHNQE |
| | | EFDKTLTVSLLRYYSALFKLVNDFEIYSLAKGNPTVVNLDVLVDDSSSPYKFRGFYNFGNMIGQIHGLTHNQIKN |
| | | EEIMIRNKIAHFDIEMLLKKPLLAQIKLDVQRKNLIKFIEDRGDMKSVLGYDAINDFSMKMVHLRTKIKVNSDKL |
| | | QIMMGLLTNVKTPNDFYNIYKVKGIESINQHLLDVIGVTESEKKIEKEINEGNKRAVK* |
| 47 | JW13149 | MMETIKQEIGKEYNPFKTEDKHYFGGFFNLADNNIKEAFNEVSIRLSQKGASPKTLIEKYTKENVSLVDYERFVIL |
| | | LSDYFPIIKEIDQINKDEKDNKGILIPKTKNDRIKDFEVILVSMIDAIENLRNYYTHFYHESIEIDNKMFYFLDKVLF |
| | | KTVLDTKRNYLKEDKTKEMIKNTLKEDFNKIFNLKVDEYLTKDNLTSKKIKKARKYGNSFDIGLTKEITASIYND |
| | | AIKYFTYDKNEKSILSDSRKTAFNERDYFYRDKNFDLPISSSGIIFLLSCFLNRKEIEDLKSNIKGYKGKVTKNEEPT |
| | | LEKNSIRFMTTHRIYSIWHYKGLKNKIKTSESATKETLLMQMIDEVSKVPNVVYQNLSKDLKDSFIEDWNEYYK |
| | | HNEENTENTINSKVIHSIVRRRYKDKFNYFAIRFLDEFVNFPSLRFQVYLGNYLVDSRPKESANVITEREIKKKIFV |
| | | FGKLNEVNQLKNDFFSKIEEDKLETNLEVFPNPSYHFPMENSEELKSANKIGIYLSDIENLLEPFKKHLNKNTSQE |
| | | KISLIEDITQQNENIKIGQPLAYLSMNDIHSVIFEVLKNPIIKDGKIDGKSVEKKIKNQIYKQINEIKNKKEESKIIKNF |
| | | RKKTESDLDLKKMIDDLIKEIKITDDKIKKLDENKKEYFDYQKNKTAKKRNYILQNSEKGEIATWLANDIKRFFP |
| | | KEFKEKWKGYHHRELQLSIAYYDTQKQNVKLLLYGLDYRKEIPMLDFSKPNFLDFYEYYLKKRKPQLYNLLSD |
| | | LEKLERGESINKEMLLSKYFKLFRKSNYQNKSLDDKINTALANPIFIERGFMDGNPTVIPNTTIKGNESRFANWFV |
| | | AYKQFDEYQKFYNTEEYFLELKDKTPNEIKKIQSQIYTQKKNDWAIWKMISYIFKDIFNQDLQNVSLSELYQNRE |
| | | QRLENEKKSKEGERKQNFFWNKTVDLQLNNKINIPEVKLKDIGNFRKYEKDERIKVFLSYNDITDWMAYLPND |
| | | WQEKHTIKPINVIDIQIDKYEKIRQHHLLKEVQNLESEIYNKAINKEELLQEDIKGNKNPNFKKYIVNGLLKQIKG |
| | | LNVENFKIPNSDKEFNALSEETLRSYTELEQKVTLLVLIRNKFAHNQLPNKDVYAFTQSIRKRQKNETYAEYYFN |
| | | LFEKIRGEI* |
| 48 | JW13150 | MEENKVRFTNTSNVKRPKSLLKQMGHKSTFLLDDTLIVTSVAKGKNEDQKGKEQAASKSKVAPQSHIELLSTID |
| | | GEVLGGRKELLATTVSRQEVTISGKIYNLNTKTPESTTVTMANPAVLLQKNSSTTQVARKTDYLGIKSALEKEFF |
| | | NDFSNSGFNDNVRVQIAYNILDIRKILATTINNIVSMFYNLDRTTGDIADETKKSTNTDIIGMIKTYLKYDQAKNS |
| | | NDPSIKKTFNKAEELLINTKAYHTYFDRLFTTPTRQAFKAGKLKDTNDKKSNLPCEFSKLSEKDQEEFKNEAITH |
| | | NYDVLRLLGLGRQLVVHEKYKSDEHNKDNSIESLFNIDKIIRNSNDTMDLLKRLDNIYLESAEQIDNSFKKSSAT |
| | | NCLILEELYKRIGKAKTQAELYKDYYRYAILKEDQCLGISFNRIKEAVLEKLKEEQRYKNFEYRNDNKRFYTLCN |
| | | FVLWHYMVDLRDRNETRAGHQQTDRNEIEELKSKLRACCDNNGDGGENQKYAIYKEFVNFHWTDLRTSINTL |
| | | YDLCVKYKYTAKNYRIDSKIQIDNQLSSSANNVKTFVKYIYFLSEFLDGKEVNVFFNELVEKFSNIAEIVTTVNDN |
| | | INNSSKKSPNISFLPQYSLFQEAGTVADQLRNARNIKHATLRARINDKVADKDSKMSPSKQTISDALYMLIDEND |
| | | TIENYKQLIWDEKESGTWVKKATGNNKVRNFIINNIIMSKWFMYISKYSDPRNCSKIMHNKEAVVFAMSSIPDA |
| | | QIEKYYKRVTAKSVQYGNKTAEEMRSIVIDALMNFSLKPVLTGVANNDYSNENRERDIGIIQLYLTVAYIVTKSL |
| | | VNVNKRFLLAFSCLERDVPLILNGKSFKDANILVLSEKFIAEDQEIEQKYKLAKKNRITPNMSKEDVKNVFKELN |
| | | AEYKSKHYAESLTRPRDPDSKKDKSCAYLVNNLNEAKKIENVLMDFKNKVDHLNVVKSAPEYMSNIKNIDSFY |
| | | GIFCFALQNILADKMIKWSVNEKNSKEDVEIINSYGQELKKKLDSRHTFDRDAMWIINMPFAYNFPRYKNLSSEY |
| | | LFYDKEYEKTKK |
| 49 | JW13153 | MKIPQIIENNKHLFGAYSTMALSNIRNILDHIATLADIENDFYSDSDSYWYHPCIKIIDPRNNCTDAAKADFVIEKI |
| | | KSHFPFVPIVAEAQRQSDILRAKKQLDIAVRNRDYQKQEELKHRQKSLLSVTNADIYRVLNNMLWVLANYRNQ |
| | | TVHYLISDIKFEDGSDFLNYNEQPLSRMLNNYFTIALRDTANKYSYSPEALSFIQSSRYKTENRRKILDTDFFLSIQ |
| | | HNNSDTNPKNLHLSGVGVALLICLFLEKKYVNVFLQKLPIYGPYKKQSMEANIIRQSFSIHTVKLPKERIVSEKSD |
| | | FTIALDMLNELKRCPKQLFSTLSYADQNAFRIVSSDMSDVLQVRHTDRFAQLSLEYIDRRELFSDIRFHLNMGKL |
| | | RYLKTADKHCIDGVSRVRVLEGKINAFGRIHEFEARRKEQGFVEWHEQAARLIKTNKNIEIRDFEHVKRDDATP |
| | | DSYPYIIDTYTHYLLENNKIEMRFGDYLPKLVKVDDQKWTVYNEMPTCCMSALELPAMMFHMMLCGSEATEH |
| | | LIKAEVEKYKKLFGAMANGTLSKENIGNFEIAMEDLPKKVIDCVNRKKSGKSFVKQIKKEIAEMLADTNLRIVRL |
| | | KSDKRSVASTQNKMGKRGFRSIQPGKLADWLAADIVKHQPSLFSGDDYGTDRITGMNYRVMQSTIATFNAATP |
| | | EHSLDELKRVFSAAQLIQCQEREHPFLQKALNRNPQNTIDFYEFYLQAKRAYYQSLRQSIENGEDVKLPYLNPDR |
| | | NKWVRRGASFYSTMGDIYLEDMPIELPRQMFDKEIKKALAQLPSMKGVDMKQSNVTFLIAEYLKRELKDDFQP |
| | | FYQWNRNYRFTDMMICEENCDTKALASKFIPLELREEIWEKRGELKDAYKRWAFGRLNKNPDIKRLSPAEKDEL |
| | | LEKRIAKCRNEYQKSEKIIRRYKVQDVLMFMMVKSMFAQGVFTAESKEFALSTITPDAERGILSEVVPIDFKFSID |
| | | GKTYTIHSNGMKIKNYGDFYKLINDKRMKSLLNIVTHSTIEKDLLEKEFSTYDDKRPEAVEIIFEFEKAAYEKHPE |
| | | LKELLTPERKFDFRALLEELQAKSDLSQIDARYLSQIRNAFNHNAYPRNLKITSNIPEIAQEMVNIFRITDPYKKGKK |
| 50 | JW13154 | MESLIQQEENKARNLIDDPQYFGGYLNMARHNIFSINNHLASYFGTSGLSNEEQIPDSFLLSADKRRNQETKELIK |
| | | ENLLFERTLRFMPIFRIFDTERLPKEVRLNTENMEKDFDEMRKCLKQVFSELNIFRNDYTHYFSTNTETTRKLQVT |
| | | PELEEFLSRSFKLAIAFTKQRMRDVLKEEDYSLVEKKVMVLPEERVITTEGIVFLACMFLEREYAFQFIGKVVGL |
| | | KGTHRNEFIATREVLMAYCVKLPHDKFISEDSKQALILDIANELNKCPLTLYDVITEDGRKQFRPELTDDAKKSVI |
| | | ENCINEDLEVDYDEYIQILTKKVRYKNRFPYFAFQYIEETKLLDNFRFSIDLGQFVLAEYEKRMAGEPTSRIVVEN |
| | | VKAFKYLKEVTDEDVVLGLMKSNPPSNRFLQFSPHYNSDNNKIGISSKPQTSVVKSKGKIDSKIEYKIKQPLPRAF |
| | | VSIHELPKIILLDYLSKGSVVKIINSFIRTNKSIILDVDKIKEIKEKLPNNEVWFPFRRMTDSKKHSAYSEKQKAEIL |
| | | RRKKLLNEILSSYSLNIKQIPSRIIDYWLNIIDVRKERSFSDRIKLMKRDCADRYKAVKRNEKDKTVAIPKIGEMAT |
| | | FLAKDIVAMIVDEDKKSKITSFYYDKMQECLALYANIEKKNLFIHLVTNELKLNEKGGHPFLKRINLNSITRTKEL |
| | | YEIYLSEKAYKLNSKENDESWMSMTFYHLEWNEKAKKKMTKVVYPVNDEQIPYTIKQWNKPQQPLFDRFTNE |
| | | QGKSSDWVRLKPLDLPTNLFDEEICVQLRSRLQEAKIPFDNISNYSELLKLWWKSRDDSTQPFYLLERQYNIYDE |
| | | SVRFTLNSKNKFKDYYQAALKKVFNQKKAEREAKRRFNKRLPDIQFKDVERVFKHAIAETEKQIRIIQEEDRIML |
| | | LMLEKLMDDQQLKLGKAETLLSSTVPVKQKIGDKTNSSRVIVAQRKQKHYTVLRKYQYDRRLPELFKYTTEKE |
| | | ELKLEWLEDELKAYNKGKLKVFDLVFSLEKRIIEVAKQDIIALLDKSGSNDSKFNVDHKPYIKWLENMELINSKE |
| | | AGFMKKIRNSFSHNQFPECQTVELMISLKSDIPIATQIVNEYERLILRISSQIEKMK |
| 51 | JW13155 | MEKELIVNENRERTLKDDPQYFGGYLNMARLNIFNISNHIAKRFGFTILPNEERISSSFLCNKALKNADWNHIHSR |
| | | TKRFLPIIKVFDAESLPKTEYDFLMWEGKNFALMTDTLKSVFSEIQEFRNDYSHFYSTEKGTIRKTKISPQLASFLE |
| | | VNFKRAIEYSKIRLKDVLREEDFNLVERLKVCNNGSEITTEGLVFLICMFLEREHAFQFIGKVKGLKGTQYPSFIA |
| | | TRETLMSFCVKLPHDKFVSEDPKQSLILDIVNELNRCPKTLYEIISEEDRKQFRPELDIKSVDNLLKNSGKEELLDT |
| | | MEFEEYMESLTKRVRHSNRFPYFALRFIEEMKLLNNMQLHIHLGKFELQKYDKQLNNEPFERAIIEDAKAFGYL |
| | | KDFNDENDIKGEIVSGKDSEGFAQFSPSYFLKYNKIGIRYKRDIPHLIVDKNNSKGAYNLKQPLAKAFLSAYELPK |
| | | IILMEYLQKGRAGELIDDFIQLNDNKIMTWDFIENIKSQLPDWKTFNKKSFGLKDKKGYQTEELKELNTRKNQLN |
| | | KLLAPEGLNHNRIPERITDYWLNIGDVKKKRSFSDHIKLMKREGMERLKQLEKFKKEGKGKIPKVGEMATFLAS |
| | | DIVNMVINEERKNKITSFYYDKMQQCLALYADSEKKALFIQIIKELKLNEEGGHPFLKKIDLQKISKTSQLYELYL |
| | | TEKVNKPIREEVFKYDRKKGYKVTKIILRDKSWMGEMFYTTEKSEKTGKDITIVRMPENKTEIPFTIRRWEEKQK |
| | | TDLKVWFHNVTKGKDKTKDGPKAISLPTDIFDDEICTLIKIQLDKINVKYKENLKYNELLKLWWWKSREDDVQ |
| | | NFYHSKRLYKVYDEEINFTPDTKSTFVEYYEKALNSAFEKLSKARKKERKTHRALPDINKKEVEKVFKRAIGETE |
| | | KDIRILQEEDRISILMLEMLMDNATLKLKNISYLLNDTTRIQQSITAHLSFDENGESIKEGTKPTITKTIIADRKQKN |
| | | YTVLRKFIFDRRMPELFEYYASNEVALDWVTDELRAYNTAKQYVFDNVFNLEQKLIEKDETGIKAMYTNKEGT |
| | | PITGNIQYKPYVRWLENKGLINKNQKRFLTMTRNCFAHNQFPQKKTVLRIVPNLTDNNIALQIADAYKNITDEIL |
| | | TKL |
| 52 | JW13156 | MDAEGKNERTLQDDPQYFGGYLNMARLNIFNISNHVADKLSIALLKNEEDINNSFLCNSNQKLNWSKAYAMTM |
| | | RFMPLLKVFDTERLPEHRTSQKDKEKGKEFGIMCDSLRIVFRELNQFRNDYSHYTSTEDNFTRKIEISPELAEFLT |
| | | CNFKRAIEYTKQRFADTYQNEDFILAERMQMVKSNIITTEGIVFLTSLFLEREHAFLFIGKVKGLKGTQYKQFKAT |
| | | REVFMSYCVNLPQDKLISDDYTQSLTMDIINELTKCPETLYNVLDESARKQFIPQLGEVETDNVRQNSINEAELTD |
| | | ENDYAQYMEDITKRVRHSNRFPYFALRFIDELDLLGKFKFQLDLGKLKLNTYPKPYMGEDVDRAITENVSAFGK |
| | | LSDHQVKEEVQAKVVKDYSSVQFDQFAPHYNMECNKIGISRKSETAIVIKPSETGVKSAYKLKQPIPEAFLSIKEL |
| | | SKIVLLEYLSPGESQRFITDFICTNSDKLMNLDFIKEVKGKLPVFDKPFYKKVTIKRKSYTKHELTDLEARKNLLD |
| | | QVLVTYGLNTKQIPTRITEYWLNIRDVRNQYIISERIKSMRKDCTTRLKSLKKLDETGKGKVPKIGEMASFLARDI |
| | | VSLVIDPVKKKKITSFYYDKLQASLALFADPDKKKEFMHLVKDELNLLSVGGHPFLINIDFTTIHYTRDLYKKYL |
| | | EEKGCKITNDRKDISWIFNTFYKKEPGKNSSLPVTKVKLPLDKNGNPDSKRLPHSLYSLGNRKTDNSIEEIEKWLS |
| | | NVKHGKMKTDCTKPVDLPTNLFDSRLIELLQKETGDTSLAPKLNGLFVTWWKKRGDSTQSFYNDKRNYVIYDQ |
| | | EVCFLPNTKKLYQDYYQSALDRAFRLKREERRQERLLNIRLPDIQLKDMKKIFNITLSETEKEIRILQEEDRLLLLI |
| | | LEKLMEKEIAGTLNLKNVNDLLNEQVDICQQVKTYLSFKENGELASKDERMQITKNIISYGSRKNYGLLRRYVH |
| | | DREMPELFEYYPTDDIPFEAIKQELEAYNKAKQIVFDKVFELERALTERFKDEVMAHFVNRDGGSRLKGNIQHSP |
| | | YLNLLESKGIISKDAHRFLKAVRNAFSHNQYPHKKTMEIFVKEWNTSNFANQIVDIYKQKIDEVIDNIKK |
| 53 | JW13157 | MEEANRYIYGAYFNMARDNFLNTIKLLADKMKLGATSGFGKDGNEVNDINELFGDKNTYANIENVVEFYFPWI |
| | | KALEGRFSLDKGDRNLNDMKMFYKSVLTAFFTAVDSLRNKYTHYSHKDLNIREIKIECTLGGKDYCIGLLNALD |
| | | CIYDSAVNLLKLRFMADEDEVAHLRRCKAVNKKVVVRTEKDGFYYRLSDNGEVTEKGVIFIASMFLNRKYGFL |
| | | FLKQLEGFKRSDEKRYRLTLEAFLAFSNIKPVDRLKSDKLDRASLGLDMLNELTKIPKELSETLSVDCLYKYLAS |
| | | DGEDDLRSRIRYQDRFVPLALEFISQSDEFKDFRFYTYVGNYVYKGYIKRLIDGTDKERYLSDRLCGFYKSVNDA |
| | | SSDAIAQKYGVEIKDSNEPDYMLPDSFRPHVLRATPHFVINNNNIGIKICGNDCLPVVNGKGVESPEPDYWLSIYE |
| | | LPAMLFYAYLREKNGKLLKDYKSIRELIEDVEKKADEKNDRDKGALMARHIDKEIIWTQTKLDEVKRLEEKKV |
| | | AAYGKKGRVVLKSGRMADLLAHDMVRLQPATKGSDKITGANFQALQVSLAYFKRDILADVFSRAMLTTGNHR |
| | | HPFLYRIDVSHCSSLRDFYVAYLGERRKYFEDVAKKITKNKLNTPCHILRRLQREGSGEEAGKDVKPKFLPRGIF |
| | | TGSIKSCLEKSALNINIRNARNDVKPAINAAYLIFMYYKEIEKGEFQGFYGEKRRYDILEEGKPLDLDERKKALAS |
| | | IKPAKIDVSEANMPMSKEEHLMRKRYHAVCNNESAIRMYQVQDILLLLMAKDIFKKALSEVVMSKKIGLENLN |
| | | GIFDAPVNFVKNFDNIKLTATGIKIKDYGKVCRLGTDFKFNSLIKAFHKVYSKSVEMDYSDYLKEEEEFEKYRLN |
| | | MVKLCREVERGITEDLHLSLDGKSHLSFNDDVIKSYNDKYNVFNGDDLTFFINARNMFMHGDYKYECVKYVVS |
| | | EHFKGSLNDVSFAKETYGHFCNLLESMRKKTGLRIDI |
| 54 | JW13158 | MTETKSKREDIAKPPAAKSRSKAAGLKSTFAVNGSVLLTSFGRGNDAVPEKLITEKAVSELNTVKPRFSVEKPAT |
| | | SYSSSFGIKSHISATADNPLAGRAPVGEDAIRAKEVLEQRVFGKTFPDDNIHIQLIYNILDIRKILSTYANNVVFTIN |
| | | SMRRLDEYDREQDYLGYLYTGNSYERLLDIADKYAVDGEDWRNTAAGISNDFEKKQFQTINGFWDLLDMIEPY |
| | | ICYFSEAFFCETTVKNPDSGRIVPCLEQRSDGDIYNILRILSIVRQTCMHDNASMRTVMFTLGQNSVRDRKNGFDE |
| | | LAELLDYLYDEKIDIVNRDFLRNQKNNIELLSRIYGSSADSPERDRLVQNFYDFRVLSQDKNLGFSIKKLREKLLD |
| | | SPALSVVRSKKYDTMRSKIYSLIDFMIYRKFSENHVAVDDFVEELRSLLTEDEKESAYSRWAETLINDGFAQEILV |
| | | KLLPQTDPAVIGKIKGKKLLNDSIAGIKLKKDASFFTKIINVLCMFQDGKEINELVSSLVNKFANIQSFVDVMRSQ |
| | | GIDCGFTADYAMFAESGRISRELHILKGIARMQHSIAGLGDVKIYSSDDKFHGVSRRVYTDAAYILGFGERSEDN |
| | | DGYVDDYVSSKLLGGADKNLRNFITNNVIKNRRFLYTVRYMNPKRAKKLVQNDALVVLALSGIPETQIDRYYK |
| | | SCIEKRSFNPDLNEKIAALSEMITTLKIDDFEDVKQNPEKNANYEAKKNQRISKERYKACIGLYLTVLYLICKNLV |
| | | KINARYSIAIGCLERDTQLHGVDFKGAAYMTRDVFIAKGWINPKKPTVKSIKEQYAFLTPYIFTTYRNMIAHLAA |
| | | VTNAYKYIPQMDRFKSWFHLYHTVIQHSLIQQYEYDRDYGRKGAPVVSERVLQLLEQCREHSNYSRDLLHILNL |
| | | PFGYNLPRYLNLSSEKYFDANAI |
| 55 | JW13159 | MNIPTIIEQKKQFFGTYSVMALLNVQTVLDHIQKLADIEDIPCYDENGKEKDKRENLWEHSVMLYINYKNNSIQK |
| | | APEKTALIIDKLMAYFPFLKIMAENQRTHKNKKYQQNRLEVNSDDVFDVLNCLFRVVKKYRDTTTHYMLHDEC |
| | | WDDNSKFLRCSEQILATMLNKYYDVALRNMKERYGYTTEELAFIQNHRYKIEWEPDSRKRPQSNTRFFFSLVDN |
| | | NGDANPDTLHLSGVGVAVLICLLLDKQYINLFAQQLPAIQKFEPQSAARKMLVRSMGIHSIVLPKDRIHSQKDN |
| | | MSIAMDMINEMKRCPKELFDTLSPSDQSRFRVTSSDHNEVLFLRHNDRFAQLALQYIDYCKWFNGIRFHVNMG |
| | | KLRYLFNAEKNCIDGINRVRVIEHALNGFGRIDEMEDLRKSEVGTFANTGVAIRDFENVKRDDADPNNYPYIVD |
| | | TYTHYILENNKIEMLLTDTNSLPTITEKGGKWYVGKKAPTCRMSIFELPAMMFHAILLGNQATEKRIKTVCANYS |
| | | RLFEALKQGAVNKDNIASFGIAEEDIPQKVLDIVNGCAQGKDVNIFIKKNIDEMLHDTERRLERLREDREAVNAN |
| | | DNRMGKRGFRQIQRGKLASFLAEDIVKLQPTLCNGTDKLTGLNYRVLQASLAVYDSGGMNEARQQFKALFVK |
| | | AHLLGTDPHTCHPFLHKVFARSIPDNTIDFYERYLAERESYLSTIQKKVAMGKAVNVPFINRAQSKWSAPTQEAL |
| | | GNAYTDDMAIELPRQMFDDEIKAHLKTLPEMADIDFDNANITFLIGEYQKRVLHDNVQHFYSWQRNYRYIDML |
| | | KCETDRKNCLCSTYTSVEEREALWGRRKECIEKYRQWFTRKTSNDRGRSLISAVDIEEILDKRLSNARNEYQKCE |
| | | KTIRRFKVQDTLLFILAKKALTNFADYNGERFCLNDITPEGDKGILSQLMPMIFRFEKKGKVYTIKSESMKPKNY |
| | | GDFFVIANDNRICNLLDLIEDNIVSKEDIDKELQKYDQCRPEMAKLVLDLEKWTYSKSPGLMYKEKVQFKDVLD |
| | | ELCKHTNLTERQKFVLGKVRNAFEHNDYPEKGILEITTLPQIAARMKDLFDEYAEVE |
| 56 | JW13160 | MKEKIKNKSSIIRIIMSNYDDKGLKEFKVLYNKQGGVDTFTCKTDIVDGTIIFLEIEKHLRDFGDDFSWDISSDGKS |
| | | VEITKLINGKETRKYKVSIKNSSTKDKKNLVELEVEDLKESAIDRRRTKSSTKRVLLSKDVMERYAEIAFSKKER |
| | | WEEIDSQKIYKVKRFLDYRSNMLIYFQFINDFLTKGIPDELDKNGEIKQLELWKLIDDDETISDKNLNQVSKNLYT |
| | | YISQEIKDSQTRAENNREKNKEKEHFKEFYAFNDISEESIREDVKKFIYLYANLRHNLMHYNYSFFENLFEGKDL |
| | | VIEKTKSLLSSTLDLNIFKELSNIVELREENKTNYLDDETTIRVLGKEKKAKTLHKIYSILCSRKNGFNKFINSFFST |
| | | DGIEEEFLKSEIKKDFLERLNWVEKSLIEKINNPPSDTKLKYKNDKTIENMTKEKEEKLELISLLNPQVSDYKTENF |
| | | TPYYWDIHQSPSYKKLYNDRKVLVSELSKLIAIGINSDTKKRITDLNAELLKIKIKMEKITKLNSKIRLQYKLQMA |
| | | FGFIYANYSKVYKEKRVLNINGFVQNFDPTKLNKEKELESRLIYLKAPYNIFEDNKSLDFNMKIVENIPVSEKSIFR |
| | | IKPENNLSKFYILSYLLLPVELRGDFLGYVKHHYYGIKNVDFEEIPDIKEDKPNENSDSFFHNLRLFEKNSKKFELI |
| | | KYRLVEFGNLKDHLPRIYEKFGIKPDVLEYIENSGNKDSKLFDRNILLPIMKYYQHIFKLLNDIEVHALLRFSEKD |
| | | SISLDESIKECSKGKFLNFGKLLFLSRYGLEAKKDNKFKDIFNRENGLSITKDDAKTERKKYFEIFETRNKIAHLNY |
| | | KQLFHDLLFDSNININKELEGIIQETKTIGLNAQTLGYNFLNDFYMRKEMFISNQKKSSMTLINNPLSKDKDTKEI |
| | | GLLKLYGLSKSQPKDLILAKYKELMNLIEKTEDSILKKKDFLPVKEVSITVKKSTPNKKGIMVELPEILQIKDLNE |
| | | MDLLAYASNIRGKLHKDSSDLFGIYKKLTIKELKKKLINLFIKGEKRYLNLELVNKTGYMAIYESTGLYPKSYEIL |
| | | NHEISFSEISMKNWYEHDFKPIFQIDGSLPNNTDYKNGVFIYTSPYEFRDKELMKKQRTVHKRDIEKTFYNENDM |
| | | DYTGIYNQKIKALY |
| 57 | JW13161 | MFNDSQGNQIEQNDKSFLEIFLNQARLNAHITLCHISDLLGENTVDEDSLAIMPVLRFLESDKDAVKSERTFELVE |
| | | KHFPMLKVIYGSGPKKEEVTFLGRTKKYKEILGCFLKAVNGKRNEYCHAYSKSSGDYDIKTLTRHLENCFDASV |
| | | RKIKEIRSLEEEEVFHLRRKTSEGKGKEKRVVDNPNFHYHFKNEEGQLSEKGLAFLVAMFLEKKDAYLFLKRQH |
| | | GFHRGESPGEKATLESFCVFRIRLPKPVMTSDVDKNGLALDMLNDLKKCPKELFELLAEEKQQEFRVTDSEDGD |
| | | EDGNEILMRRYSDRFPYFALRYCDENEVFKNIRFHIDLGRYYFKFYEKDTIDGNKYQRALDKRLKTFGRIKDVK |
| | | NKVRQEWGDIIKAPDEISDEQNEPYKTNSTPHYNLVDNQIGFTISGDCKLPDVKQPEGKILLQKPDAWLSIYELPG |
| | | MLFHGLGYGFKKTESLIEQYIIRQRKICEEIFRTGTIPANAGEFLPNAIKEIGTGNETSNEYAKNKLDRKLSDTRQRI |
| | | RAIKATKNRIADKSNKPGKRKFFDIRAGKLADFLARDIMALQEFDPAKEGKDKLTSINFQVLQATLAFYGAKKN |
| | | TLAGMFKRIGLLEGDNPHPFMKEIEPGKHNSIVGFYEAYLDRKEAYLEECRKGENLDNYQFLRPSRQRYAKGKR |
| | | DLKTITRQLLDNPVNIPNGFFREEIREFVCEQDAGLKSRKMNTAYMVGANFEKTYGGQQPFQYYQRNYPVVLK |
| | | AQEYCKKRENRGIAEVLRSIAGEMSYREMEAFIKKEIPDNGRFEPENLKRNLLNGCKDFKKNERLLRRYKVQDM |
| | | VAFAMVEKTLKEQLSFDGNVVKLEAITPTEESLFKKPILCSTDIAITFNTKPTHYDNGYVGFIQEKYAGHYASEG |
| | | NKIILKYRIISENTKLKDIGKYRRYLYDRRLPGLLIWKYAPNAQSDIEIKYSEIEEEIKAYEQYRLRIAEQLYVLETK |
| | | VIDCYICKDEPGEDYISFNGIIDTIKAEQAGSEEKCDTLLSIRNAVYHNQFPAFMDAIEKTEGDNIAGKMLSITEDY |
| | | VKCTVEQMNEGDKEFVNV |
| 58 | JW13162 | MNSVELKKEEAAFYFNQAELNLKAIEDDIFDKGRRRTLLDNPRILAEVENFIFNFKDVTKNAKGEIDCLISKLREL |
| | | RNFYSHYVHNDNVKVLSKGEKPILEKDYQFAIERTASEDVKLEIVDKRDKLTDAGVLFLLCMFLKKSQANKLIS |
| | | GISGFKRNDPTGQPRRNLFTYFSVREGYKVVPDMQKHYLLFTLVNHLSNQDDYVENVQQPYDISEGLFFHRIAS |
| | | TFLNISGILRNMKFYSYQSSRLKEQRGETKSDKGYFEWIEPFQGNSYFSVDGQKGVIGEDELKELCYVLLVGKQD |
| | | VNKVEGRITQFLKKFKNADSVEKVEDDEMLDRENFPASYFAKTRVGSIKGKILNRLKKAIESKKITGVDVKAYN |
| | | KMKEVMAFINNSLPVDEKLKPKDYKRYLGMVRFWGREKINIEREFRSKGWSKYFTSGFWMAKNLEEAYKLAR |
| | | QKNAELFNKLKTAVEKMNEQEFEKYQQINDAKDLASLKRLAQDFGMKWEEKDWQEYSGQIKKRITDRQKLTI |
| | | MKQRVTAGLKKKHGIENLNLRITIDSNKSRRVVLNRIAIPRGFIKKHILGWQGSEKVSKKIREAKCKILLSGKYEE |
| | | LSRQFFKAKDYDKMTQINSLYEKNKLIALMAVYLMEQLNIQLKEYTELGTLKKTTVDFKISNKVTEKIPFSQYPS |
| | | LVYAMSREYADNVHDYKFSEKDKPFLGKIDAIEKERMEFIKQVLEFEKYLFDNSIIDRKKFADTATHISFKEIVNE |
| | | LVNKGRDGNELTKLKDARDTALHGGILTGTSFEETKSLINEMKK |
| 59 | JW13163 | MCNEIQTNQTGQKDNSLFGIYLNQARLNAYIALCYISELLGEKTVDEDSLSEMPVLRFLDSDKDIVKSKRTFELLE |
| | | KRFPMLKVIYDGEPKKEQDDKFLERTKKYKEILTCLIKALNDKRNEFCHANTKGGGGEYDIKQLIRYLNNCFDA |
| | | SVRRVKDVRGLEEEDVFHLRRKTSQGKGREKRVIDNPNFYYHFQDKEGKISEKGLAFLASVFLEKKDAHELLKK |
| | | QRGFKKDDTPGYKATLESFYCFRIKLPKPVMKSDEDKNGLALDMLNELKKCPKEIFDLLPKEQQEEFRVIDTSDD |
| | | NEDDNEILMRRYSDRFPYLALRYCDENELFENLRFQIDLGRYYFKFYEKQTIDGNTQQRSLDKKLKTFGRIKEVK |
| | | DKVQQQWQDVIKSPDEIEDEQKEPYKANTTPHYNLVDNQIGFVMSRDCNLPDLNQPDGKLKLQKPDAWLSIYE |
| | | LPGMIFHGLTSDFKQTEILINKYIGRQRSICKKIFGTGSIPQDAGEYLPEALKDIEENDTKRTSYAKKKLCRILEDTE |
| | | QRIKAIKKTEQRLVDKSNKPGKKQFFDIRAGKLADFLARDIMILQSFDPAKDGKDKLTSINYQVLQATLAYYGA |
| | | KKDSIADMFKTIGLLDGDNPHPFLKEINPSKYNSIVEFYKAYLNRKKGYLEKCEKEGKFDEQFLRPSRQRYAKGK |
| | | RDLKTIAKQLLDNPVNIPKGFFQKQIKEFVCVREPSLEELQMNTAYMIQAYFEKEYGTQQPFCSYQKTYPVVSKS |
| | | TEYCKKAKNKEIKEVLRKITAEMSYRELRKFIDSKIPDGRYEPENLKKNLLDGCRDFKNNERLLRRFKVQDMVM |
| | | FMMVEKTLKDQLAFEGNVIKLKDITPVEDSPFNKPVFCSSDVSASFNTNKKYESGYVDFIKKNYKGLYISQGGRII |
| | | LKYRVTSESTKLKDIGKYRRYLYDRRLPGLLIWKYPPNIEGGIEIKYADIEEEIKAYEKYRKQIVQQIYMLEKKAV |
| | | DHFKLEIEPEENHIPFNTVTDTIKFRLAGSEEKCDTLLNIRNSINHNQFPVFDEKIENAQGQSISERMFSITENYVKY |
| | | ITEHITESDKDFAYV |
| 60 | JW13164 | MLCPRAIFSTLFNFVHMEAKHTIQYFDFTNIKHKHFFAGLLNTAQHNFDLSLRELAKRIHNKDTTKGLITIAFNNS |
| | | VSESDFKARTNYLSESLAFIKLMNYENRAEFRDTLLVLYNTIDGLRNYYTHYKRDKITIDESVYKLLDYLLLTSA |
| | | KEVKINRAKSEEYKKYLSIKYQDEFEEIIDESNKKIEEDNALLKKGERKTGLIKPQDEVNHVLNKVFRSFLYKND |
| | | KGEDELRDNASSKNKEDTDLSTYGFVQLLALFLNKKQTESLLSYTKYLKDTRKLKFVTSRWTLTFLCYKDIKRL |
| | | FKSDYSNDSLLLQMISELTKCPNEIYRQLTPDKKQEFIEDINEYYKDTISFEGTNEQALVSHEVIQKRYANRFSYFT |
| | | IRFLDEIIDFPTLKFHVNLGKFNHDTTKKKFSNAIETERSILEKITVFTKLSTATNNKKDYFSKPENKELENDWLEF |
| | | PMPSYQFNKNNIGIWLSIDKKSGESDAPEERENSKLSKYKIAEKLLLKNTIRKPIAFLSFNELPALLYSLLIEKKTPI |
| | | EIEKIIQSKIIEQRKLISSFDKTKIISNKRLPKKLIRASHLDSTTDVKKLEKDIKTEISKDPIGDIRKHYKKEIKNVNEF |
| | | TNSEKGKVTTWLADDIKRFTSKETRENWKGYQYAEFQALLSFYDINKDKPKSFLEKDLNYDYKKDNSFNGLNF |
| | | NLKTLFDFYNSYLKNRREYLNNLLKEIESSKDEIHEKVFIGFTKRLYEIKPIDIQKNVFLKKIPLNLPRGIFDDKPTS |
| | | YDKGGKDNTEIASWFTLSSKAENAQLYYCFPKVYSYPTTKLKWDNRKRRKIQKSSIATQLISPKERLKQQYLKD |
| | | LPKTVQVEIYKNEKSIRKIMREDFFILEMVKHLLKKSSIKGLDKLQLKDVFLTKEEKNRRIVESLKQSQRVKGDK |
| | | MENVVNDSYILSQQVAISILNDKIIDTVQIKTIGKLRRLEEDKRVRQLIEYFPDKPSWTVLEIQSEIDDYDRVRKFE |
| | | FFSSVHVLEKLIYKDAKKNNAKEGLLQNNNHNFRKYLSYYYLNEDRDAFNSLPNFDTITIDNPSIKSEWYRLYLL |
| | | VKIRNKFSHNQLLSKVDFDSLKEIISMNSGEKIAAFLNRAFDKLIIVK |
| 61 | JW13165 | MNNNATIQKQSVIYAFDKSEDKHFFAGLLNMAELNLVIALQETLIRTIRNTKTSNNAKVLIQQLFTEDIMQSDWN |
| | | YRVEVLSEFLPVTKALQPQADKATFIKTLTLFAETLNTLRNFYTHHYHPPVEIDSRLFEILDAVLLHSAVYVKKQ |
| | | RLKTEDGKTMFPKQYEDDLRTKLAIHNEGVKQKRISEREKPENKKIPKNKFNEIFNKKNKTYSEADFVNTYLNG |
| | | VFHHLLYKDRKKEGLDQLKKDAASKKKDENYFSKNGFVLFLSFFLDKKNLEDLFSHTEHFRGTHELRFMVTRW |
| | | VFSLHAFRDVRRVLRSDYSNDALLLQMIGELNKCPKELFNILSTEHQLEFFEDLNEYYSNNEGGDESQPDSLVTH |
| | | TVWRKRYDDKFSYFALRFLDEFAGFTNLKFQVVVGNYVHDSRTKELAGINKVTYRTIKEKIKVFGKLSEIDRLK |
| | | KEYFLKKNQEQQVSDPTTETTELSEGWEEYPVAHYKLDNNNIPIYLSIPGFQKDSTPKEYRTKRLNKKSILEQLTT |
| | | TGFIESEPIAFLSVNELPALLYQLLFNNKSAAQIETMLIQKITSQVSEIKRFNTESTIDKHKIPKRLIQLASTIATSTSI |
| | | NFVKLLKDINSEIDTCNSRLKETDDNLNKSKYRRFNNKQRGKVATWLADDIKRFAGKDIRSNNWKSHLHGELQ |
| | | ALLAFYTERKADIQLLLEKELGLPLDSKSLLSTSFSENTLEGFYVKYLKTRREILTDFKASVDKTKNEYPNSLDTS |
| | | TIEPLFDIFDKRLYVSSHIEKYKSDLLQKPLNLPRGIFDTATYFPLQKGDKSNFQPWFEVAAKTEHYQEFYNFPRK |
| | | YETIELNPVSGKGLLEQNNNKPVDPDVFKNEKRLREIARQDFYVLEMAKYMIKHMYPAIRTSQNYTLKDVFKTK |
| | | QEKEQIKLRAAQQSQKKAGSGGEHFTDDSHLLLRRFPVTVAIGNKKIVDYVALKDIGKFRRLEKDNRIKLLLSYS |
| | | EQEIWTVKEIEDEITNYEHIRKQSFFKEVHVFEQEVLQKVKLKAGIDTHPEILEQNGYPNFKHYVAYHFFNNDTIT |
| | | FDKFVKVDFQNEKSRKEIVESKNMLLLKAYFLVLIRNKFSHNQLPEKVDFLKMNAYIKFSGEKIGDYLLHFFKES |
| | | IVY |
| 62 | JW13166 | MSAMTKGLRNCKGCVNMKKKMSLREKREAEKQAKKAAYSAASKNTDSKPAEKKAETPKPAEIISDNSRNKTA |
| | | VKAAGLKSTIISGDKLYMTSFGKGNAAVIEQKIDINDYSFSAMKDTPSLEVDKAESKEISFSSHHPFVKNDKLTTY |
| | | NPLYGGKDNPEKPVGRDMLGLKDKLEERYFGCTFNDNLHIQIIYNILDIEKILAVHSANITTALDHMVDEDDEKY |
| | | LNSDYIGYMNTINTYDVFMDPSKNSSLSPKDRKNIDNSRAKFEKLLSTKRLGYFGFDYDANGKDKKKNEEIKKR |
| | | LYHLTAFAGQLRQWSFHSAGNYPRTWLYKLDSLDKEYLDTLDHYFDKRFNDINDDFVTKNATNLYILKEVFPE |
| | | ANFKDIADLYYDFIVIKSHKNMGFSIKKLREKMLECDGADRIKEQDMDSVRSKLYKLIDFCIFKYYHEFPELSEK |
| | | NVDILRAAVSDTKKDNLYSDEAARLWSIFKEKFLGFCDKIVVWVTGEHEKDITSVIDKDAYRNRSNVSYFSKLM |
| | | YAMCFFLDGKEINDLLTTLINKFDNIANQIKTAKELGINTAFVKNYDFFNHSEKYVDELNIVKNIARMKKPSSNA |
| | | KKAMYHDALTILGIPEDMDEKALDEELDLILEKKTDPVTGKPLKGKNPLRNFIANNVIENSRFIYLIKFCNPENVR |
| | | KIVNNTKVTEFVLKRIPDAQIERYYKSCTDSEMNPPTEKKITELAGKLKDMNFGNFRNVRQSAKENMEKERFKA |
| | | VIGLYLTVVYRVVKNLVDVNSRYIMAFHSLERDSQLYNVSVDNDYLALTDTLVKEGDNSRSRYLAGNKRLRDC |
| | | VKQDIDNAKKWFVSDKYNSITKYRNNVAHLTAVRNCAEFIGDITKIDSYFALYHYLIQRQLAKGLDHERSGFDR |
| | | NYPQYAPLFKWHTYVKDVVKALNAPFGYNIPRFKNLSIDALFDRNEIKKNDGEKKSDD |
| 63 | JW13167 | MSNEIGAFREHQFAYAPGNEKQEEATFATYFNLALSNVEGMMFGEVESNPDKIEKSLDTLPPAILRQIASFIWLSK |
| | | EDHPDKAYSTEEVKVIVTDLVRRLCFYRNYFSHCFYLDTQYFYSDELVDTTAIGEKLPYNFHHFITNRLFRYSLPE |
| | | ITLFRWNEGERKYEILRDGLIFFCCLFLKRGQAERFLNELRFFKRTDEEGRIKRTIFTKYCTRESHKHIGIEEQDFLI |
| | | FQDIIGDLNRVPKVCDGVVDLSKENERYIKNRETSNESDENKARYRLLIREKDKFPYYLMRYIVDFGVLPCITFK |
| | | QNDYSTKEGRGQFHYQDAAVAQEERCYNFVVRNGNVYYSYMPQAQNVVRISELQGTISVEELRNMVYASING |
| | | KDVNKSVEQYLYHLHLLYEKILTISGQTIKEGRVDVEDYRPLLDKLLLRPASNGEELRRELRKLLPKRVCDLLSN |
| | | RFDCSEGVSAVEKRLKAILLRHEQLLLSQNPALHIDKIKSVIDYLYLFFSDDEKFRQQPTEKAHRGLKDEEFQMY |
| | | HYLVGDYDSHPLALWKELEASGRLKPEMRKLTSATSLHGLYMLCLKGTVEWCRKQLMSIGKGTAKVEAIADR |
| | | VGLKLYDKLKEYTPEQLEREVKLVVMHGYAAAATPKPKAQAAIPSKLTELRFYSFLGKREMSFAAFIRQDKKA |
| | | QKLWLRNFYTVENIKTLQKRQAAADAACKKLYNLVGEVERVHTNDKVLVLVAQRYRERLLNVGSKCAVTLD |
| | | NPERQQKLADVYEVQNAWLSIRFDDLDFTLTHVNLSNLRKAYNLIPRKHILAFKEYLDNRVKQKLCEECRNVRR |
| | | KEDLCTCCSPRYSNLTSWLKENHSESSIEREAATMMLLDVERKLLSFLLDERRKAIIEYGKFIPFSALVKECRLAD |
| | | AGLCGIRNDVLHDNVISYADAIGKLSAYFPKEASEAVEYIRRTKEVREQRREELMANSSQ |
| 64 | JW13168 | MKVVKPCGNSVVLAGGEIRRTIQPSITSITSVNAKQTHRPAIDFLLNNEKFFLAQWASMLDKIISKPHQANQASPN |
| | | YATYEARENLSKAFYVLWKDKKLLNNFSHKDNQRKSFNQQWGLKVHPYKKPNKPTANNKPEAIKKAGEISKG |
| | | RWYDSLVTKSKPNKNTAKHHKSATIHYKNTAKIIYEHLYVKQRSISKRSAYPQGYIANRANSIEKSVHSFTLKDN |
| | | KLATLCINDQHRINRLTKQSISIAQPSKYRLDGLIKDLKIKTKQYYQNKVSAAELRAKKTKRPVKPPLENQQLPL |
| | | KEAMICLYEHYARIFGTVTKIETIIIKEPELWQYHCLIKSTLKQRYPKQAVLNKIYPVITEDQLHKLAQNIETNKTL |
| | | AMAIRLGKVVHYSQDNLGYEDIEHSDIVNSDYWGSAGQTLIKQKESFAQHWLIALSVANGSLRGLLDTQDNADI |
| | | YLSRGINTALEGIPTDFEARFKLLFGNHHQLYQDDLQRDELLRQLYGTLSQLRHASFHFKSEQSFNQVLLKKVTL |
| | | DTTLDNNTSVSQTLAKNINNYIDEHLAQQQALVKKTLEASFVTKYLNQTQCDQLLSSLQNNQAIATLLPKFNKL |
| | | LHRAATTEQSIIAVANQVELQNNPNQQCAFICLKQLYEQDFWRWLLKQKSSTLNHHIDQAKIRANKQANKLNA |
| | | KNTLEHQKIMARMQQLRGINAQESLLAYFSYLSAQTASFFQIQTNTYQHNAQAASEHSNFIEQLKQDFLIRAFKQ |
| | | YLKAEKFNYLLHLGTPASCVIDIDKITKKTPVNNPLLYLSLHLIPIEFVNQLSLQIKKYTVLTGSEKFGNIHNDINL |
| | | YLTMHNAILDIKWLDNRDELAKFYDNKALFEQVFSQDNKHLIPVKGLREMLRFGNLSELGVIFERSIVEANEINS |
| | | LQDKQKNIAAIQAKKAKAHQKLSQQKDINKQDKADYKSDVDKVSEYNHLKNQVYLHNHKQTHQLIMTISGRL |
| | | VGFSRRYERDIYFVLLALLTLNKVKTDELSQNFLASIRSGQIIKASNQLGEQYANIKSKLNLLFQIEKHREIRNRFA |
| | | HFDDLRKAESLNITQQINEARQLMSYDRKSKNSVVKSILNIVKKDNISIDFTMDNQHNLTLKSIKADSIIHLKDKDI |
| | | SEKPQGKKYKNAVKKLFENTV |
| 65 | JW13169 | MNVIESFLPLNTKEKIDQLMAIIKSEPKAMNQNAWHMIFGNIGKSKMQMYFIEKYPQSLFAAPGNYIPSCNGQGQ |
| | | EEKNPQQPQVQQHERQTDIPVPPTSLKDNRFLYATYIEMAFHNMFLNIKHIYGVVFGRDIMAEAKANYEALNPE |
| | | KKWDEDFANEFLVWKPMFEAFNNGNVEEKQKVGEMLSRHFPLLVPFTDFTNHESNYKNLTIVGILRRLSQVLR |
| | | VLRNLYSHYRIELFENQKKVYLDNEYLIIRCTMNAYMGARRVIKDRFSYDEKDMRCTDQYQFVDERGRRLKEK |
| | | VEIKGFRYKIGEKGKDSKLHFTPFGLVAFISLFLEKKYSKILTDKLRLIPIQDQHIINEMLAVYRIRLNAQKLNISKD |
| | | ADLLALDIINELQRCPKDLFSLLSPSDQKKFRHESEANDEVLMVRHSDRFPFLVMKYIDDCQLFDNIRFQVSLGK |
| | | YFYKFYDKNCIDSETRVRALSKNLNGFGRLSKIEAMREGCWEDSIRQYDDIHKNTVDEKPYVTDHHAKYVING |
| | | NRIAMRIIREEEKAYLPELNAEGVRNLAPTCWLSIYDLSSLAFLLYLTNGDFVEGIIENTVENYHKLFKDIENGTM |
| | | LPVANEEELDSLLCRNYGGIKQKDIPKNMLDYLLRKTSDARKRFVEFSKSIIDRMISQTEYKIEQIKRQEEQCRQN |
| | | KIGKKSYVIIKDGKLASFLANDIMLFQPNDSENSNKLTGLNFRILQSTLATMKECGIDQLKRTLTSAHIIGEANDE |
| | | YCNPIVMKMMRKRVPENTRDFYKSYLKARLEYLNECKRKADYENLSFLHSNRSKWQEHDDEFCRLLAGRYLH |
| | | DENGGTEFEKSINLPSGMFDKYIREKLCEVPALKDIANDETKNVSYLIYAYFKNVCNDDCLFFYEAGRSYRLFKL |
| | | LGMNGSKTAQDIRLMLNRKHKTSLYKEIDKYLISIPLAEREQEKARLMGLLKGMKNSETDFKRFKIQDILLFLIA |
| | | KKLFKDNVQDDVQHTAFESLKLKSLKDTDGLSQKIRISVKIKSKNGYEKEIKKPELKLKNYADFYKLLNDRRLP |
| | | ALLDLVREHSIDYDSLVDEFGGYDKVRPNVMKDVLKYERKYYDNHADSDESLDFGSMLDNSSFNEEEKDLLRK |
| | | IRNSFAHNTYPKYYIVNKAGDPHLPEKAIKCSEKFSSDINNE |
| 66 | JW13170 | MAFDKPRPKRRQARSDFYITDKAIMGGYFNLAQLNFFKTLMEIFTKAGIDVSKIKQDNSPKYLMILIKKLTHDDE |
| | | KIDDKKWADALDLSNECLLKLQQLLYKHFPFLGPVMGGEASYNIYKLKDGHPEVKVANDVMRGVKLEDCLAV |
| | | VRHFALCLNDCRNFYTHENPYNPIEAQKEQYLSQNKVAVWLDKVKDASRRIIKQNNQLTSQDMEFLTGIDRMK |
| | | PQDKVDDFGNIMTDRRGRPIKEYVEYEDYYFRIRGKRYLVDAAGAKLVDQEPRNALSDFGVVFLCTLFLESDQA |
| | | RRMLDELRLFETGPYDGSRDGDEFKNDILREILLFYRTRVPRGKRLDPMDDTTLLAMDMLNELRKCPMPLYDV |
| | | LSREGQGFFEDEVKRPNDLTPEVAKRLRSSDRFPYLALRYIDLNKCFDNIRFQVQLGKFRYKFYDKTTIDGEQVV |
| | | RGLQKEVNAYGRLQDVEHYRQEKYADMLQQTELVETGEEDITIANFIPDTPQSSSYLSDRTASYNIHNNRIGLFW |
| | | NMPGEQEVLAGDEKMYLPDLNVDDNGKADVFLPAPKASLSVRDLPALVFYLYLQNQHPDLTPAEAIIQQKYNA |
| | | LVSFFEDVSTGRLLPVKDIAELEAAIDKYGHLTVHEIPEKLRDYLTGVAGTEDDDDCADRLDCYAMGILEKRYR |
| | | RVAAHIDQLKEARKMVGDKMNRYGKKSYIDVRPRRLAEQLAQSIVDWQPATDNGRHKMTGKNFQRLIAFLAR |
| | | YTDEVPVQEIKDLMTRARLLDGPVAHPFLQQVLDKNPLNIEVLYDLYFKEEKLHLENYLEIETNKQGEEVFRKK |
| | | ASVDLSRIPFLNSGRLRFHKRDSASFQRLAARYMQVDGRQSTILLPDGLFTPHILSVLKSEYRDLQDHLQDQELN |
| | | RNASYLMSVYLECVLHDSAQPFYLSNSNDSPFARRYDLFAILQNKKKGAELLPNPLAPVEINRRLTAKTPDGSAK |
| | | LIERDIKALLDDMARKTEERIHDLQQEYSRKKEKQAQEERVKLERCFMRKIADVKKQAQEERINLQRRLKHKIT |
| | | DVKKNERTIRRYKTQDMAVFLMAKQMLANQLSWQNQEGEVPFKLANVCNEGFLRQTMKVSFPYEVPLKEDK |
| | | TKKVTIDIVQDNMSLKNYGLLYKLYHDERVVTLLQNIVDQQQISFAELTAELSLYDQNRSKAFSNFQQFEKLAFE |
| | | TNKEALTHPEHRGFFLEKVNKKNQVIRYERRNNFDALLDLLDAIEAHQLDPRERQWLTSVRNAFCHNSYYKID |
| | | MQQIEKKLPTIIQQIVEIVQRITDKASHPSNR |
| 67 | JW13171 | MSTSDSFYSFGVDYTKTYDQQPDCRRHVFGAYANLARHNMTLVVNTIMQAIGMPTFNENIISDAFNSTHRKEID |
| | | KLDNIKKVLLQERLYRHFAFLKRMKLEDSKKKTVQLNTLLETLSDFTRMMAYMRNFYTHYNPYNSRDEKEKQ |
| | | GDLNKKMGERLQFLLENSLQMLKDTEKLDHAKNEVLSSLRVPERKTIRPGDTEYSNIHDLYFTKNKRKGIKYDK |
| | | DTKVFTQQVYILNPAYPAKMTDSDGYLSLAGMVHFLCLFLEKKIAFELMDEVGFTQQCHFNGRDAEQQLLYIK |
| | | EVMCMNRVRMAQPRLDSEMSDTALALDMINELRKCPEPLFKVLDKEARNEFKDDATVQWEMEHNVEARPGE |
| | | DEENNNNEDDIEEPDETIREDKAVPRSTFVRWNDRFPQMTLSYIDMQSVLPTIRFQLYLGKYRFAFYEHDKEYSV |
| | | DNAERLRVLQKEMHGFGRPQEVKANLKDKWKDYFEHKTIENGLTMKEPDKAGQAPYVTEQNPQYAIDEKSHS |
| | | IGIRWGEWSSGHGGSHYGDLDKLNMFIPCLPKPKADEDKDKQRNHAEKLLPPKCMLSLYELSGLVFYHYLLKK |
| | | CGKETGQVERHIITYHDNLVRFLTDVKEGTIIPVTDVDDPKGYLEKMLMESYALRLSDIPERIKKYLLNETKSRE |
| | | YRLKESAKKRLEIRLKKVMKDLESHQAKKKRIGTKENKFNRKRSTIKTGKLAKDLIRDIMDWMPRGSVGRMNL |
| | | SGESYMAIQAALTMLGQQFDRGKANVEIYDLVDILVKAKLIMPDSDVEGADFNPSHYHPFMDDVLTEDRCGPE |
| | | TSLEDVYEWYLEEEIAHIKRLISSINKVSTDSLLMKQYNLIPFLHCGRERWNKVDDEAIKKLAEKYLNNPIQLPNS |
| | | LFAKPIFELLKEVAQEKNYDRLSQALEAASQDANNLSKNTAYLIGLYFEMVEGDHSQAFYNSCPADDGTVSPYR |
| | | HVYRVFKKLYGEIIENTNRTTSPAYSKKEISDILAGREVNGKILSDRISRYVQKAVDEFKKQKDDDLKQYRIEIKD |
| | | AAFNENRVKHLRKKAYEIFEEVDVKVDQKREEFERLTDQYRQEVEAKLKKLLKKVQLNERAILRFKTQDILLL |
| | | MMSRDILKMGSDNEFKLENVLSESLLEKRVDFSWKVSVNDQTKDNVTTYKTIEQKNMKMKDYGQFYKFASDH |
| | | QRLESLLSRLSPSVFQRAEIENEFSYYDSNRSEVFRLVYILESFALEMKPGLKDDNNAKEPWFNYIRKGKPSPKRN |
| | | SFIELLEVLIAGADGILDNQEKELLREIRNAFGHNTYDVDFETIFSGPRKSKRKVPEVANGIKDNMEEKTTNVLEK |
| | | TKK |
| 68 | JW13172 | MKQNDREKNSKIKKSAAKAVGVKSLARLSDGSTVVSSFGKGAAAELESLITGGEIRKLSDKAILEITDDTQNKNV |
| | | YNVKSSRIPNLTARTDKLSDKSGMDDLGFKRELELEVFGQSFDDSIHIQIAHAVFDIQKSLAAVIPNVLYTLNNLD |
| | | RSYSTDDTNGKKDIIGNTLNYQHSYDNFDKEKLGEFKEYYNAAKDRFSYFPDILCVLEKVNGKDRYQPKSEEDA |
| | | FNVLSSVNMLRNSLFHFAQKSNDGKARIAVFKNQFDSGFSHITSTVNKIYSAKIAGVNENFLNNEGNNLYIILNAT |
| | | NWDIKKIVPQLYRFSVLKSDKNMGFNMRKLREFAVESKSIDLSRLNDKFLTNNRKKLYKVIDFIIYYHLNKVLK |
| | | DSFADGFVAALRASQSEEEKEKLYAQYSERLFADEGLKSAVKKAVDMMSDTKFSIFKKKAPLDKALIENIKVNS |
| | | DASDFCKLIYVFTRFLDGKEINILLNSLIKKFQDIHSFNTTVKKLSENNLIINADYVDDYSLFEQSGTVARELMLIK |
| | | SISKMDFGLDNINLSFMYDDALRTLGVSDENLPEVKREYFGKTKNLSAYIRNNVLENRRFKYVIKYIHPSDVQKI |
| | | ACNKAIAGFVLNRMPDTQIKRYYDSLINKGATDMQAQAKALLDCITGISFDAIKDDKHLHKSKEKSPQRSADRE |
| | | RKKAMLTLYYTIVYIFVKQMLHINSLYTIGFFYLERDQRFIYSRAKKENKNPSKNSYLNDFRSVTAYFIPSEIMERI |
| | | EKNENKGFLKEFEEAWKTCGETSRLRKEDVLLYARYISPDHALKNYKTILNSYRNKIAHINVIMSAGKYTGSIKR |
| | | MDSYFAVFQHLVQCDILSNPNNANCFKSESLKPLLLDMRFDGTDEKLYSKRLTRALNIPFGYNVPRYKNLTFEKI |
| | | YLKSSINE |
| 69 | JW13173 | MGKKKHDKQEPIANRHILGTYFNMAFENFNRTMYILCEKKLKISTSEYGTPDTEHLIADQPIQLRKALFRYLPFL |
| | | APIMDDWVGYQLRSKKSKGNGGDENITQKQKTAKLDSSIEDIVSQLNIFAQELRYCRNKYTHKNAYTLAWEDE |
| | | EHLQREKQIAKMLEVLMKAERELLKNRKDREKKSSAMELFLKDGDQYYYRTNKGKTVRNKQFYFNPASFELT |
| | | KKGQVDYDTIALTDFGRFYFCSLFLRSRDAERFATEMKIFIESPWPDIKDKDSNPTTYTPSPENKIMREVMQMHRI |
| | | HVPREKRIDAVPSKGTLLMDILEELRRCPKILFDTFCPTTKSSFNHIVIQDDGTKTSTLMVRHQDRFAQLALRYID |
| | | QNGLFPNIRFQLRLGLFRFRFYDKTLIDGTPCIRTVHKELNGYGRLQVVEEKRVTKWGDRFQKVTTKENEGYEIE |
| | | QLQPDTPQTDPYVTDWRSTYNIHTGRIGMSWGEVNVGGKTEAGSALLPKVDRKSNGDDCYYIPDLPTLKSDDG |
| | | KNKDRKVNIPQDAPLCTMSTYDLPALLFYQYLIDSYGRKGADEKKLPTSENIILEKCYALKALFKAAEDNTCTPK |
| | | SLREKERELGLSDNEIPKKLLSYLDGEKHNPQQNRKKHVEHVLNGIINDINGRLESFENKEKRIKAGGRNNRYGN |
| | | PHHADVRHGSLARYLMRSMLRWQSVDVESPHFGKLTSLNARTLMGALAAFGSGDERLPLNEILTAANMIGGNN |
| | | PHPFIEEAINGADNIEKLYVAYLKNEKKHAEKILRNIDEHLQSLPPFLRASDEQNHWGVDLEQQAYINKYAAKLS |
| | | ELPIQLPDGLFTKYIVELLHNATKKYSELKIMDSIVDSDKRTTDKGGHKIGDNPSSSASHIIACYFEKVLGDKSQK |
| | | FYDSQTKDYKRFYENISKLNPRRERNKALIPDYFTAKEIQEKIKEARKSVKGKNDDDATTLRDKLNQVEEKERAI |
| | | RLFRVEDMVLFLSAKQMLMSVVGRSLTEDVNQTNALHESKDNLSQLRIKAKELKLGQFGFDEDFRFLSEGDNE |
| | | EERKEKDQTESLLEYIYPYKIRKEGKLVGTINIKQKGLSLKNYGNIYSILGDDRLKSLMEGLFEIGVSEVTFGDITS |
| | | EFANFDQLRSTILKLLQDNIEEIAFNRNISVLNNPRNRNFYSVKDKDGNQLKIKITDPVTNEIIALSKDAIRNQFPEL |
| | | IKMLEEYEADRVSINELRKAVAHNHYPKDISKLKPTKNKKLQLPNIAHLANENAKKIIDAAQKASAEKEKKSE |
| 70 | JW13174 | MANTQFKLKSVDYSKPYDEQPECKRHVVGAYANLARHNMTLTVNTIMQAIGMEIFDENNIGDAFNSSHRKKID |
| | | ALDNIQKVNLQKRLYRHFPFFKRMKLEDEDKKSVQLTTLLEVMSDFTSCMAIIRNFYTHYHPYNSPDEKKRQLE |
| | | LKKRMGKRLQFLYENTSQMFKTTEALDHESNEVLSALRIPEDYIEEILPGDKEYDNLFNLINDPRTSKFKKQGLK |
| | | LNQQTKVITRKKVRFVRNPDYQAYMMDETNGMSDVATIFFLCLFLDKKVSFDMMDEVGFTKQIKFNGPQADR |
| | | QLLYLKEIMCMNRIRMTKAKLDSEMTDTALALDMLGEVRKCPKALYEVLDKDARNEFKDDATVQWETEHGK |
| | | EAVSTEALASESDEDELTDKNTPRSTFVRWDDRFPQMALRYIDYQGLENDIRFQLNLGKYRFAFYQHDNKQSVD |
| | | NEERLRILQKELHGFGRIQEVEEKKNEKWKELYDEKTVKDGLTQKKTDEAGQAPYITEQYAQYAIDEKSHSIGL |
| | | RWEGWNKKDKSKGHYGDLDKLKMFIPYVPSKPKEEERQTNQAEDLMAPQAMLNLYELPAILFYQYLLDKYKS |
| | | DTSNAENIIKTAYSNLVSFFNDVDQGLLAPAEDADENERISKISSLLEGYHLQMSDIPEKLRNYLCNKGIDYDLK |
| | | MEKSAIGRLEEKKKHVEKALSSYKDKKKIIGSKDNKFDKMRASIKTGQLATWLIRDIMDWVPNKSSARNRLTG |
| | | QNYTALQAAMALFGQQANSNRSYLESCFRNAHLLPLDGKPFNKNTHHPFLEDVLQAMEDETLESFYEKYLEFE |
| | | TKHIAKVIKTLKNGTDFDDYYQVPFLHCDRQRWAGPDKNQAAQYLKRPLQLPNGIFTQPIFDLLWKIDNLELHK |
| | | ALEESQQANGDNPPLTDNVAYLMRLYFEFVEHDHAQPFYYTTTTDGNKSHYLHIYRIFKKLYGQPIPHSNKTTV |
| | | PAYTIDEIRHLRKQAIADIEEYVDNEIEKWRRTKQDRFESKLRKKLKKENDKRYKKHEKQLNINEEIQKAVKKET |
| | | DTMRTDMTSKMKRQLRNVYDNERTIRRFKAQDMMLLIMARNILKAKSNDKDFTDGFCLENVFSDSLLDKPIDF |
| | | EWKVNIKGASKTIKQEGMKMKNYGQFYKFASDHQRLESLLSRLPDSLFLRAEIENELAYYDSNRSKVFRQVYIIE |
| | | SEAYRLKPGLQDDANASKDWFYFTDKKTHKQRPIRNSFIQLLEILAAGEDGILDNDEKVALQKTRNAFGHNTYD |
| | | VDLNVVFKGKEDKKKIPEVANGIKEKIETQTEELKRRSK |
| 71 | JW13175 | MTDNTNKYFKLKEVDHTQTYETQTDCQRHVVGVYANIARHNMTLAVNTVMQSLGEPLFEENSIADAFNDTHR |
| | | KDIRDFENIKKLELQERLYRHFPFLKRMYKDKEVSLKDLMETMSCFTQSMAMVRNTYTHYRPFESNVEEQNDL |
| | | KTKLGQYMEDLFLESRKVFKKEERLDASSNEVLGRVYKSIKNDKGEKYVLDFNYPAYMKQGKEGLSDVGLLY |
| | | FLCLFLDKKAAFDLLNRTGFTQHAKNYFDGNEEQTLLLLEMMCMNRIRMVHSRIDSEMSNEALALDMLNELRK |
| | | CPRPLYEQLGKDDRDSFKDVMSTQWENESPDNEDAPIDAEGKSIPKTTMVRWDDRFATMALSYIDTMQWMDD |
| | | IRFQLKLGKYRFAFYNHDGDHIAGNLLRVLQKEMHGFGRIREVEKEMNEKWGALFEQKYEENGLERKDADQA |
| | | DQAPYITRQRPQYAIDDESHSIGMRWDFTRPKYWDFTKPSYGDLANKKMFIPHLPGNPKPAEEGKHQTNQAERL |
| | | IPPQCTMSLYELPALLFYEYLRRKLRNETMDSAEQIIKRYCNSLKSFFGDVSRGKVFPDDENELAEALNKDYGLR |
| | | LSDIPERLKNVLLGNSFDGNEKLKKIVKTFVEERKNEIKEMKRCLDEGINHKLVKTGQAAQWLARDIMIWVPTQ |
| | | TREKLSGQKYSAIQANLAMVGNWEGAVSYGDLRQLFTAAGIIPESNLKNPNQHHPFLKDVLNGLLNDREHSVK |
| | | SLYQRYLDVESSHISSLELKLARNKNCLDSFPSLHHNRMRWKQQDAAALAQSYLTVKNKAKDSEIILNSEKPIQL |
| | | PKGMFAERILAMMKELAKNNGALNEGLEKIEKQEKGLKGNTAYLIGLYHTFAENDASQPYYNYERVVDEGKL |
| | | AKSERKRMEQTIRRYKTQDILLFYMAREILALPVGNDNRQRFLLKNAMNNDLLSLPIDFSWTVRLKDGSAKTIT |
| | | QKAMKMKDYGNFHKFASDGKRLASLLEQQAETEFARADIEAELARYEQSRVEVFRQVYILESHAVMKLGVAD |
| | | DCDIKAREDLFYDSAKGKRCKLNSFNQLMQALGIGEKDRESLTKIRNAFAHNYYVDLKGIGFWGSVPKIPNVAS |
| | | KTKEWMVRKCNDLYGPRFGG |
| 72 | JW13176 | MAKKKTAKQIREEQKAAKMQKIQTFVKAKEAKKENEKTASAVKLPVKKNRKKSLAKASGSKAVLIVGNDIIMT |
| | | SYGERCLANLEHKITPELNTESLTVNNVLNNVNAKDGKNISFTGRISGLSVSSDNPLHFHQTEERKAPKPGTDLL |
| | | CLKDKLEQWYFDKTFDDNIHIQLIYQIQDIEKILASHVNNIVFALDNILHATESETGDFLGMGYMRATNTFEEYR |
| | | DSAISNVKKSYETFETYIKKKELLYFGEAFYHGNTCRKPEDIYHILAMVSSLRQFCFHSTFDDKSKSIKGSWLYQL |
| | | DEQLSDEFKETLGILWAEATEKLDRDFLKTNTVNLHILCEIFDKEKQEDIINAYYEFLMKKSFKNLGFSVKKLRE |
| | | CMLEEAELNEFKEDKYNSVRSKLYKLFDFILVHHYKNDSSPESTALKNLLRTCLTDEEKEKVYVQTAKELTALY |
| | | SYHFKEASVSVNARNIKEYQEKAQVYHISIADKISSTGIGLFSQLIYMLTLLLDGKEINDLLTTLINKFDNIIGFLDI |
| | | LDKMHLDTEFKKEYQFFNQENCKYTAKELRTINSLARMQKPSANAKKVMFRDALRILGMDNRSDEEIDKELDR |
| | | TMPVGADGKFIKGKQGFRNFIASNVIESSRFRYLVRYNNPHKTRELIQNQHVVKFVLEGIPDTQIKRYFEVCKDK |
| | | DTPDTSSKSAQIDVLAGILTNIDYKIFEDVPQSAKINKDDASRNRAEALKKQRYQAIVTLYLTVLYLVTKNLVYV |
| | | NSRYVMAFHCLERDALLYGVAFPPVEKKWGKDYSQLTASLLKDETYTTYGHLKNQKGHRKWHVLVSRNLEN |
| | | SDKKAVHWFRNISAHISVIRNINEYIGEVQEFHSYFELYHYLVQKMIAKNNWYDAKSQPKTAEYLENLEKYHTY |
| | | SKDFVKAYCIPFGYVVPRYKNLTINELFDRNNPLPEPKEDKEKKEV |
| 73 | JW13177 | MKKRLSNPPARRQDSNPPSACKLYLTSFGKGNDAVLEQQIDTAADYAVSPISAEPSLTVTEAAEARVKFDSKRP |
| | | NVKKIQLTADNPLHHSAGNPASTRRDILGLKNALEERFFGRTFDDTIHIQIIYNILDIEKILAVYATIISAALNHLMD |
| | | ESSDVSQSDVIGNIATQNPYAIFKDPQSRYDYRDPSLTPDQRKDATRIRKKIDANRAAFQALLATGRLGYFGFDY |
| | | KPPRKDEKDKKKIKAAETQEKRLYHLVALAGQLRQWSFHGGDKPEDELWLYQLDSQLVGERKEFLDTLDYYY |
| | | GDRFREINDEFIEQNKINLAILEELYPKEDFPEIAALYYDFIVVKSYKNMGFSIKKLREQMTTLEGASVITAQNMD |
| | | SVRSKLYKLIDFSLFYRYYKDEARRQRAVNILRASTTDEEKEAFYAAEAEWNWNYFRGRFTDFCLKIGDWVKM |
| | | DVDASRWNGILDLDSYRRTSSASYFSKLLYAMSFFLDGKEINILFTTLINKFENIASFITTAKELSIDVTFHENYAFF |
| | | NQDCDGYAREIDVVRNIARMKKPVPGARKTMYRDALTVLGIPEHMQADMFDAELEKMLEKPKDEKGRKLKG |
| | | KNPFRNFIANNVIESTRFIYVVKFCNPDKVRSLVNNTVVTKFVLGRMPETQIDRYYQSCIANPDSAAPLAARIDAL |
| | | ADMMRNMRFEDFKDVQQKSGDPIENMRKERFKAIIRLYLAVVYQLVKNLVNVNSRYVIAFHCLERDCQIYTGK |
| | | SVSKSKKYFTLIDVLLEEGDSSRSGYLARNVRMREHIAHDVDIGKSLIITTKSTDSATGEVKYSKLRIIGFYRNNIA |
| | | HLTAVRSFADYIGDIARIDSYFGLYHYIVQRLLQSRDYTAEAFPDRINPYYASLDRYHTYVKDFVKALNSPIGYN |
| | | LPRFKNLSIQQLFDRNELQPEPKDDKAEES |
| 74 | JW13178 | MAKKKRMSAKERKQQQINLRIKKATEDSTKKVNTTVAVNNKPISKEIKKSKAKLAGVKWVIKANDDVAYISSF |
| | | GKGNNSVLEKRIIGDVSSDVNKDSHMYVNPKYTKKNYEIKNGFSSGSSLTTHPNKPDKNSGMDALCLKTYFEKE |
| | | IFKDKFNDNMHIQAIYNIFDIEKTLAKHITNIIYAVNSLDRSYIQSGNDTIGFGLNFNIPYAEYGGGKDSNGKPENK |
| | | SAWEKRESFIKFYNNAKDRFGYFESVFYQNGKQISEEKFYIYLNILNFVRNSTFHYNNTSSHLYKERYCKINPKN |
| | | NLKTDFEFVSYLNEFVKNKFKNVNKNFISNEKNNLYIILNAYGEDIEDVEVVKKYSKELYKLSVLKTNKNLGVN |
| | | VKKLRESAIEYGYCPLPYDKEKEVAKLSSIKHKLYKTYDFVITHYLNSNDKLLLEIVEALRLSKNDDKKENVYKI |
| | | YAEKIFKAEYVINPIKTISNLFAEKGDKLFNEKVSISEEYVEDIRIDKNIHNFTKVIFFLTCFLDGKEINDLLTNIISKL |
| | | QVIEDHNNVIKAIANNNDAVYKDYSDKYAVFKNSGKIATELEAIKSIARMENKINKAFKEPLLKDAMLALGVSP |
| | | NDLDEKYEKYFKTDVDADKDHQKVSTFLMNNVINNSRFKYVVKYINPADINRLAKNKHLVKFVLDQIPHKQID |
| | | SYYNSVSTVEEPSYKGKIQLLTKKITGLNFYSLFENCKIPNVEKEKKKAVITLYFTIIYILVKNLVNINGLYTLALY |
| | | FVERDGFFYKKICEKKDKKKTNKDVDYLLLPEIFSGSKYREETKNLKLPKEKDREIMKKYLPNDEDRKEYNKFF |
| | | KQYRNNIVHLNIIANLSKLTSTIDKEINSYFEIFHYCAQRVMFDYCKNNNKVVL |
| 75 | JW13179 | MAYDKPRPKRRQARSDFYITDKAIMGGYFNLAQLNFFKTLMEIFTKAGIDVSKIRQDNSPKYLMILIKKLSQDDE |
| | | TIDDKKWADALDLSNECLLKLQQLLYKHFPFLGPVMGGEASYNIYKLKDGHPEVKVANDVMRGVKLEDCLAV |
| | | LRHFALCLNDCRNFYTHENPYNSIDAQKEQYLSQNKVAVWLDKVKDASRRIVKQNNQLTSQDMEFLTGIDHM |
| | | KPQDKVDEFGNIMTDRRGRPFKEFVEYEDYYFRIRGKRYLVDAAGTKLVDQEPRNALSDFGVVFLCTLFLENDQ |
| | | SRRMLDELRLFETGPYDGSRDGDEFKNDILREILLFYRTRVPRGKRLDPMDDTTLLAMDMLNELRKCPMPLYDV |
| | | LSREGQSFFEDEVKRPNDFTPDVAKRLRSSDRFPYLALRYIDLNKCFDNIRFQVQLGKFRYKFYDKTTIDGEQVV |
| | | RGLQKEVNAYGRLQDVERYRQEKYADMLQQTELVETGEEDITIANFIPDTPQSSPYLSDRTASYNIHNNRIGLFW |
| | | NVLDEQEVLTREEKMYLPDLKVDENGKADVFLPAPKASLSVRDLPALVFYLYLQNQHPDLTPAEAIIQQKHNAL |
| | | VRFFEDVSTGRLLPVKGIAELKAVIDRYEYLTIHEIPEKLRDYLAGAVGTEDDDDCADRLDCYAMGILEKRYRQ |
| | | VRARIDQLKEARKMIGDKMNRYGKKSYIDVKPRLLAEQLAQSIVDWQPATDNGRHKMTGKNFQRLIAFLARYT |
| | | DEVPGHELKDLMSRARLLDGPVSHPFLQQVLDKNPLNIEVLYDLYFKEEKLHLENYLEIETNKQGEEVFRKKAS |
| | | VDLSRIPFLNSGRLRFHKRDSAYYQRLAARYLQVDGRQSTILLPDGLFTPHILSVLKSEYHDLQDHLQDQELNQN |
| | | ASYLMSVYLEHVLHDSAQPFYLSNSNDSPFARRYDLFAILQNKKRGVELLPCPLTPVEINRRLTGKTPDDNAKVI |
| | | ERDIKALLDDMARKTEERIRHLPQGFYRKKKNQSQEEMAKLERRLKHKIIDVKKNERTIRRYKTQDMAIFLMAK |
| | | QMLANQLSWQNQEGEVPFKLANVCNDGFLSQTMKVSFPYEVPLKEDKTKKVTIDIVQDNMSLKNYGLLYKLY |
| | | HDERVVTLLQNIVDQQQISFAELTAELSLYDQNRSKAFSNFQQFEKLAFETSKEALTHPEHRGFFLEKVNKKNQV |
| | | IRYERRNNFDALLDLLDAIEAHQLDPRERQWLTSVRNAFCHNSYYKIDMQQIEKKLPTIIQQIVEIVQRITDKASH |
| | | PSNR |
| 76 | JW13180 | MARKLFSNETRPHLAAYFNMACHNAFVVLSRISEKASKSCDQRLEMKLSELPVISLFDTKKPEEKEWIMNRFEK |
| | | SFPFISPAMKMNKISEAGYDDLQCSSRPYVRTTYAMKKCIVDIFNMLNFFRDKYTHYVFNDERNDNAEYRDIQK |
| | | RVVEYLEACFTASCRIVKERFLLDKELEFLTKGRYERHGAKCVVNTRSPFALRDEQGMLSDMGILYFLCLFIEKQ |
| | | YVSMLFDQLGYKGYAIYENHSELRRKYIREIFSVYRIKLPRERIVSESAIEALGMDMLGDLKRCPEVLFKTISAND |
| | | QAKFRIKAETSGEETLFIRKDSRFARLALQYIDEKEHFKNIRFHVSLGEYRYKFYEKRCIDGDTRVRILQKDLNGF |
| | | GRLAEIEKLRTERWGKYIRPFESIRIDDVHTRPYITDQRAKYLFNGNRIGLCFNTKDAQVMSAGVYMPAIDGDNA |
| | | PCVQPTCWLSVYEIPAMIFHLLLCKEDDALENTRGSDTESIIKNYVANYKRFFDDWAKGRWRRSGDAAKDFADI |
| | | ETEYSIKQRDLPQKVCDYITGVKGKPLQEKASAIVKAMRAENEEMIRRFDERYARAISPDNKFGKRSFIEIQPGRL |
| | | AAFLAEDIVRFTPSVDGKDKPTGKNFSVLQSAIAIYSLRKGYDMLDELRRIFLSAGIIGQRQGKNHPFLDKVLADE |
| | | PQDTMQLYRAYLVQRDVYLRRVNATKTYTSAYFIHTTRAKWAKRTDEYYNSLAKRYLSQPIELPRRLFEDDIKR |
| | | RLIKRHPDMAACLQQERCNVAYMINCYMQQVEHDGVQSFYAMPRRYSCFADDTYRTVDEMQSYSHIDLLSHY |
| | | LRECKNNASEEAPYIEADARRRFHALYKKYDDNERVIRRYMVQDILLFLMARDLLIKIKTDKKSFEEYRLQKVT |
| | | PEGEGILEYKVPDFAVTLTFANGEKRTIRQHNLKVKNYGDFFKFMYDERVKTLLPQVKSLEIDREVLEQELQHY |
| | | DSSRPDVFRLILKLEQFIVEHFSELKQQRVQFRDILDKVTLNTADKEVLRLLRNAFSHNKYPRLSQIEEYALPGVA |
| | | VSLQHIAEELVTKAGR |
| 77 | JW13181 | MAHRQQWNKNKASDGKKPRKASWQWKQSMTTEMKPVMASYLNMARHNVYRTLIHISTQMQIAEQGDEDKL |
| | | AKFEIWKKLEKAPAPEQMKLIKLLQKHFPVLQAFFNYEKKAGKVMVDTSAHDVRHFFVKQLLPTLSRIRNEYTH |
| | | YAAKPRTQKEEGELRPYLRQALADSVPAVFDRFAVEVVEDNKCKKKKPTQPKVVSEAVKKIFPEPFDKKGNKK |
| | | SIYNLVSGDAFSDLGIVFFTCLFLERRYTAMFLDALKPWPESFNEVQRKAVFEVFTYDHIRLPREKYDSVTPDYA |
| | | LGLEMLNELQKCPAELFDILSSKDQERLSVDIKSDDPDVVTDDGVTVKGGKVQMKRVSDRFARMAMMYIDRT |
| | | NAFKNLRFMVHLGNYRFKFYMKQCVADKEADTLRTLQKEINGFGRIDEIEAARRQMYAPMFKATVKKENAAG |
| | | VEVEELQPDHAGMKPYMTDTKANYVFDNNRIGLRAIGGDREPRLYLPSLTENGEPIDDAKQVKLYTPNAWLSV |
| | | YELPGLIFYKLLCDTYPEQAEKLGDAEEVIKSYINAYRKLFKNIRDDKFDGWDEKRYSPLTLEDLPVKIKTYILKP |
| | | EASVNPLFAKKAQIVIKEMVEATEKEIERFNRKLDRLTDREVRPDKKTDLRPGAIATRLSRDIMYFAPKSEAKDK |
| | | MTSANFNSLQSALALSKYSVKRMRGMVDELGHPFIGEAFDNYAATNFKIYDFYKIYLESRLKYLKSKQGLGKAE |
| | | LEKLPFLHPDRAKWQKRDKKGIQELAGRYLKIGEVDFPHGFELPRGLFTARAEAIMREAGVLTEPAGDRKPSMT |
| | | NLINHYFEHVLEDDNQPFYCWSKHYEVFDRLAAKKDDKHPTPQFLSPTQLCEKMKVRKRLRPSKFMLNKALES |
| | | VNNGLPDEEHIKDIYDYRITSLAKERLKKDYAEYDDNERMLRRFAVQDKLIFLMAKDILLGVGGIKKEALGKFK |
| | | LKDVMPGAKDSILETTVPFIITLTVGDTILTIRQKEEIKIKRYGEFYRYNSEPRLKSLLPYLTKKIGGNADAKLEIDR |
| | | EELEDELSRYDLKRVDVMSMVQTFEKGIIKQAGKAGIRIDDDTLINFNNLITKIGHVPYEGQGKILINIRNGFCHN |
| | | EYAKDISIPEETSLPEIAEQISTLFDTTRKRPTTKE |
| 78 | JW13182 | METTINNRIGKGEYYTEESKEFLAAYFNQAIHNVFIVLNHIAKRFGMDELSSDEELKNWLIGRQREKKRNAIDRQ |
| | | RFLELIDRHFPFLRIANADKKDAKRENDLEDNLALLITLLNDLTERRNKYSHAITHASIESNDRELVWRLYSIYDA |
| | | NINLVKRDYFESNVHTEINEDPYEKQVEHLRRFCMNNDRTKVDEKGKKKPAMPNPRFRTPFLNAETNQLSIYGL |
| | | VFFVSLFLEKKYAIQMQKNVYGLKDARDTKFKMTNEVFCRSRIIMPRVRLHSDKSTDALVLDMLNELAKAPEV |
| | | LFDQLTDPFKEKFYIESIDSLEESDIITPVRAIRKQNRFMYFALRYLDESNAFSKLRFQIDLGNYHYHLYESKINDQ |
| | | TESRHLTRKLFGFGKLIAFEQEFAPEEWKMKSKDLDYYEGATQPFIAKTYPHYHLEENKIGILFNGQAEVQWPHL |
| | | DVEEHESFPKYKRRANEKADAFLSGNELLAAAFCHHLYASIGKPNTVEKIIRDKYHALRQLFSDLKSGNLQNLL |
| | | GDNTSNEAISQLLFEKYRLTLSEVPVRLHAFLSGQEQADTKAIARGKLELMAQQNKKRIERFDAMKKAVVKVG |
| | | KAQYRTLRSGDIGDWLVRDFMRFQPIGYKRNQAGKQEPDLKSKANPKKYQLIQKSLALYEQEKNNLLGLFKSC |
| | | NLLSSENEHPFLNEVVQSMPATWQDFYERYLHARSRFLEKCIEKGIKKNSYQACYSFLKIKPQLKDKEKLYQGW |
| | | DAQMNLPTNMFIDAIHDWFRQTHHESLRTWFTQQEKPHQLIGLIRKYIELAHTDQIQGFYDMFPLRYDFYKKEFP |
| | | NGLVLHERINKQKQIWEAQLIQTKKRLEDAASKLKKVKQQVESLPDQELRFRNETEAMVYFTKLFDSSIVKNAI |
| | | LKLQRDQKALRVNTIGEKIIKIYTAKYDAIHQEVRDFKSMLTTEKMIRRVKAEDCVTLFMLTDLMNQSQITIGQE |
| | | QRTIKLSDIQPMGETQIQGILDAVQVLEQKLDFFSSDEQGKISQVKLGEWTIFSTDTKVKKQGNFKQLLKDRRLN |
| | | NLAHYILPDIAGGAIRVRRNLLEMELDQYDRNRIPIIKLMYELEYAIYKVDPFSVELKYKKFSQCLREYAQKAVL |
| | | NVEQVEHLNVLIAIRNAIMHNQYPNRDHLKAIVPFTVSEYAPVQEGLTIARQLLACAQVSVNIILSTISKFE |
| 79 | JW13183 | MNEIKQPNNSKYVYAPYLTMAACNIDAVFYHLEDIFNHKQNNAIEIDDGITQLYKLKIFQDLGISNGSNDENTPV |
| | | DFLRKDAIIKQICRNIPFFNILFDSKIFSNKSLDERCRCVDRYIRALFDMRNYYSHVLHEEIDITHQIPNIENDLNYIF |
| | | EENRNTVKKRFVLNDDDIKHLKVFHKDKKGSNSYYKFLESGDHCDKLTVNGLLFFTCMFLEKRYAYELLKKHY |
| | | GFKNSSSKMHKATLETFTVTCIRLPHNKLEVSDSKVTLGLDIANEICKCPDEIFSLLSIKDRNKFRIPIDKTDPEQNI |
| | | NEDCEDNDTQTILQKRYSNRFPYFALRYLEFMNHLPDIYFHIDMGTYFYDCYKKQLIDNSEIKDRRLSKNILCFD |
| | | KMDNAVKTFNDSKNSGLYAAKYDEKNEYRNNTFPHYHINNNQIGISLEKYIPELRGKDTRLRKPDALLSIYELPL |
| | | IVYLCLNNKGEDVTELIKNHINTSKLFFEYVASGGRYSIDNENMIIFNAGNKINSHHLPELFRKDKLNYKDLASKA |
| | | LKNQREKAQNIIDRYYTTKDRKGKILKNKRSLKTGHIADILMEDLMRFQPSIEYTAGKDKVSSPNYQALQKAFA |
| | | YIGSRYMEIPNLLRKARLTHSENSHPFIDKCISPMPKNINGFFKRYLEEKIKYIDTIVAKGDFTNAYFLRDYIKKYG |
| | | ADLETERRNAAHNLIDKPADIPRGIFKDLFSTEYASPTYHIYKELLENEDDVQDFYHLKLNFPEYINDKQKYKVY |
| | | QKINFIRACDIVSFYMMKDLIGIDNSSVKLMSSGGDKHILNLSQNVDIPVMLLRTIGKGKNTKTKINEQIISGKIKI |
| | | KDYGKFRRLIFDKRLFSYSLYFNKNEEGSEKDKLIFDDIEFEFTEYDKSRLKIFELIHKFEERVIIKNAIALPVPSNS |
| | | DDKKNKKYSYISFPDILKKSGLNKENMELLKNIRNAFSHHEYYVVKDLNANKREITKKLYDKCEKDIICITNSI |
| 80 | JW13184 | MLIIKPYGRSQTQHASDSQRNRLHLPSGQAHAEAVEQLQNNLDFVMAQWISVIDKIIRKQPQRSGKGDKKIAAP |
| | | QVSTTQFNARKALGMAALQCIEKEFSELSDLQKKQWGWKVHPYDCTNLVEPQPKDRITGRWYSRFVGAIHPQD |
| | | ITQQTVPAIVQKIHDHLYQQALRPNASTAVHTQGHTTHQLHTIARNVHALKTPKASTHVSSDSAWQSYFLSNDV |
| | | VACIYKELQDKHDQKDKTKHQSQPKPLIPTTTPKAPLFSDRATIAQHLFNHYAKVFATPSGTPTPVKDLLNEAHP |
| | | LHAQWLVHQCIKATYKALFDNASIKGLKHIQHKLPANSEAMRQLIENRRKNHDIGHLVRLGKVLHYEAAERGQ |
| | | LPRACWGEISTPAVAEGSAYWLSEGQAEIKRSEALVRSFKQAITHAGHTLSQLSDWSGNGDIFLNASEAAKQAD |
| | | AKTVEQRVALLFGRACSDLFPSCYAPSGNLQAAIQALLAQSIEAWAQLRHASFHFKGKHVFLEKLQNSLAKNNV |
| | | STPELDAVAQKLWEQDGEQRQKRIRASLEAAHCLDHLSQAQLNAVYLCIAGDKQTRIGSSLDMPRLNRVLNRA |
| | | QNTKLGMKPLTDGAPTLQLSTPAKRQTLENSPAQRCRYVLLKLLYEQDFAAWLQDTRQETIDKWIGGAIARNT |
| | | KAARELNGKGLQPQTTIHAKAERLWHEQAQAPTAKSTSQANPIARFFHDLAAATASEFRVQRHYASDGEQAQA |
| | | QAAYIDDLKCDVVARAFLTYLKNKWPWLLQDLAVQNTPSCMDALPADAHEALQLDNLWQQRLYFLLHMVPV |
| | | GVVSQLLHQLRKWQVLSTQAEASKTAVEDQALYTHCLQVMVLYLDMHDAKFDGGTAIHLDDTHRQPLKDCF |
| | | KRVEDFAACLPASANPSQPGDAHIPLRGLREMLRFGGAKVLAPVFRQHPISAAEYADWQKLQAVIKEAQKTRE |
| | | DLHAEWVKNRKGFTGQQAYAQALETCMSHRHLAHRVRLVDHLQLHSLLMAVLARLADYAGLWERDLYFVSL |
| | | ALCYHEGLDPAQVFNESGNHGNYFDGGQIVEALRNESSPSKSIQTQVAMLFGTDCKKGDPMVNIRNALSHFNM |
| | | LRHTKHQTAPAPIDLTHWVNQTRRLLAHDRKLKNAVSKSIMELLEREGLVLTWQTNHQHQLELSKVDSKKIAH |
| | | LGAKQGTEPHHSGAYCAMVRQIF |
| 81 | JW13185 | MLVIKPYGRTTTTVESNAQRKLNRKQTPWRADDVGTLNQEHDFLIAHWISMIDKIICKPKAIKAAEGQERKRSQ |
| | | PGKATTLQRDVRQLIGDAALDFFNPKPNREIWQWKLHPYEVGDAPTGQAAPNIKGRFYKAFVGHGVEPHQIDA |
| | | TQAKAITEKIRAHLMTAALKQQGNNPQSAQSESRLGRIAHQLGTITQNVHHKKDLEPHALDEAAWQTYFQKGD |
| | | VIEQIAKALAAKYNKSDKSKQPAQPTLTDRATIAKFLFEHYALVFGKTTQVRDLVREEHPLHKQWQVHQRIKAT |
| | | YKAMFDNASMKGLKGIHKRVPKDKKEMWGRIHQRANNQSVAHLVRLGKVIHYQAAEECKTVRAAWHTLQTT |
| | | LDASPYWLSDGQAEIKRSEALVRVFKLAVSHAGRSLSDLTQWEGGDIFDNEDKASKGAVAQEFGERMKTIFGN |
| | | DKESVFPSVWATDDALSEEARQLLEQTIQAWAQLRHASFHFKGRDGFLNELKRSLSSEPHTLAYPALDQLSKKL |
| | | WDEDIITRKERIKKTLEAAHCETHLAQHQLESVYKTICGENVTRYGSTLDLPRLNRVLNRARNADLKVDTTNAQ |
| | | VSLPMPAKNEVLEKSPSARCQYVLLKMLYEQGFSSWMQEKNLYEVDKWIVRAIERSNIAAHAINGSNKNNKQPI |
| | | ESIQARATRLWQSIGQKTGHDFSNDLLDGKSPIAYFFHQLAAATATEFRVQRFYESNGEAAQEQAAFIDDLKCD |
| | | VVAIAFAKYLGSKFSWLLQGLDGTQTCSINDLQKATTTASVEIWQQRLYYLIHFVPVGVVSRLLHQLRKWHILT |
| | | AKGKDRAPTPDNLYAQCAQVLVLYLDMHDAKFDGGTAHQLDDDLLKKLKSCFESEADFATLFPQGAEADSRIP |
| | | LRSLREMLRFGALDTLAPLFEKHPITHAEYERWQALQGQIDDAQNLRAELHSQWVEHKRNFDQVLQYQAALKI |
| | | CEAHRHLAHRVRLIDHVQLHNLMMAVLARLVDYAGLWERDLYFVTLALCHHHQIDLNSLFAHTDKHGNCFFD |
| | | NGQIVKALRKAESDECFTKVHTTLSALFGETYLGGELNGLRNRFSHFNMLQKKESASINLTNEVNEARALMVYD |
| | | RKLKNSVAKSILELIEREGFKLSWKTNNEHLLERASIQTKRITHLGNKAEEAHHSSNYCTMVAHLFGTT |
| 82 | JW13186 | MLIIKPYGRTRTEDNESSGRQRKLVGKQIQLQSEPVTVIAEKNEFLMAQWISIIDKIISKSPVKRIIEGKIKPCIAPTQ |
| | | RQARERIGQAAIKFQPALKELEANWKAKIHAYKITRTVSSGDPKDRIKGRSYAKFVPHGIEPEQITTQIAEEIAKKI |
| | | HDHLNHHALNSKGTGGKGKGSIAHQLGSIAQNVHQADKESGQEPSEVEWQKYFDHHDVVAAIAKAIQSNRPAR |
| | | TKPTLTPKETANTGQSPSDSPRRLTDTATIAKHLFDHFGQVFGQNAKVKDLLKPSHPQHGLWQVHHRIKATYKA |
| | | LFDNASVKRRKNMAIHMPLNKEAMMALVRHRTSNQDLNQLVRLGKVIHYEAAACGQSVRSAWSNIRPNIDKS |
| | | PYWLSEGQSEIKRSEALVRVFKLSVSHASRTLTDLCQWQEGDIFLNSEDASKKLIPDRFVQRMQTLFGRDQDAS |
| | | DPVIPSAWHSNGQLHDDAQALLKETIDAWAQLRHASFHFKGRNGFLTQLTQGLSRHAGSSPPSKVALDTRARK |
| | | LWDRDHLARESRIRSTLVGAHVETHLNPVQLEKIYTCITAKDTNRCGITLDLPRLNRVLQRAHNTLIAQENDDQI |
| | | LKLPQPAKREALEQSNAARCQYILLKLVYEQGFSDWLHAKGIKELDKWVKLAIKRTDESAQRLNTPYSDDLPV |
| | | KARANELWQQHVRTLSTSDALGGMSAIAYFFHQLSAATATEFRVQRHYESDASAAQKQAAFIEKLKCDVVAIA |
| | | FSKYLQKDFPWLLSDLDPTQSCCVSELAVHNNATPVELWQQRLYFLLHFVPVGAVSQLQHQLRKWHILSHHGA |
| | | EHESSSLGQLYASCAQVMVLYLDMHDAKFDGGIAYLLSDEQRKQLEDCFELSEDFQACFPKGSVQDSRIPLRAL |
| | | REMLRFGGIELMNPLFVQHKITHTEFDRWTQLQSCIEKFQKSREDLHQKWIDHPGDFSKQDKEAYGQALKICEE |
| | | HRHLAHRVRLVDHVQLHRLMMTVLARLTDYAGLWERDLYFVCLALCYKKNIDLTTLFNHEDKEGHNFFDDGR |
| | | IVEALRKVKKDFQFPLFSEDPSLLLISGSTKLAGELVFIRNHLSHFGMLSDQSRSKSTQLPNLTDEVNKTRQLMAY |
| | | DRKLKNAVSKSVIEILQREGLRLVWQMHDHTLQLDPNEPIQTQMIKHLGDPSSESMTESMHSKAYTQMVDQLF |
| | | GNKQLNSQT |
| 83 | JW13187 | MDRKYMTLDDKYYFAAYINMARHNVYLLLADISTRMGEKVPNDDAKLIFPILQKLEPKTTTGKNSVSPDEQLKI |
| | | IKELHKHFPFLKTMLYAYKKPMGAATEEEIQKMDYAPGDYYDLFTLFFKVLNDYRNEFTHTISQPNVKILGTEEF |
| | | RVSYVFDFPANLLYGLRVTFDTAVRKAKTRFNFTPDDTKHLLRQGKGGKELVGFKYKFQAEGSKSLTIHGLTFFI |
| | | CLLLERKYATLFTQKLQGLKDKRSRGFKATYEVFAVHCIRLPNARYTSDTSNGALLLDMLNELKKCPTLLFPQL |
| | | PAKGEQNKDQDAFRIKVEDMPGMEEEGDSGFVLLKRHEDRFSYLALRFMDEVKWFNLLHFPVDLGNYHYHLY |
| | | DKIVDGLPRIGSLWEKMIGYGRLPDYQHKFLNKDYPAEWQLLWQNHELKKENENDKSSYMPPTMPHYHFPDG |
| | | KADNIAIKITNPETLFWPAIHIDGNDKKAGKPAKKEHPDLLLSKYELFAFIFYGLLIGKDRIKKELEDKLVQYFNK |
| | | RKQLYRNIINGKLLPLALPADIPKPKDKGKKPTGYDQRKELLNQKLAIYGCTADELPDEIKCFLMGVITESFEIKA |
| | | RYRLEDLIVDTEILQERIKRQLKEKRKPGSKKDVEIKAGVLAGFLAKDIMLFQPADKTEGVKASGKPTSTLYNVL |
| | | QAKLALYGANVDTLERTFNECGLLSGSTKHPFLGQVVSRKPATIVDFYMAYLTARHSYLLKCETDKNYQAYQ |
| | | WLKNNQARKKTEPHFGQRLAAEMEKLPVVIPRGFFAESIKNELLALNNDKLNAAIVSARTPNTSFMFMAYLKEY |
| | | LDDDVQDFYGWPRVYKIFDNFHDKRTKSEMRKSLKPEPLSEEARKGQMDELKKWIKETPNDEDYGKFKKRYD |
| | | DFESNEKQIRQLKVQDITTFAMCDYLLKNINRNLINSDGQFYLKDIAPKGDKGILDKPMSDFGRVFDYCCTSKDF |
| | | AGVEITIEGKTRIFQDDLKVKNIGDFNKFIKDRRLPNLLAYLQKEQIHRAELEKELERYDIVRIEIFKIIHEFEKAILT |
| | | KYPGIDGKDFDNIISHFQQSNNLPELTAMQIKKIRNAFSHNQYPDPIYFLNNEDLKLIGTTGVELYTDYFFNFIKTI |
| | | LVSEN |
| 84 | JW13188 | METPATPNYKRFDYESDKAFYAAFLNTATQNVFIILRDISESLGLGFDLSNDDNMLNVNLWNILQSNIEPELSLHII |
| | | EKIERQFPFANYLAKNYAFYKRKERTAEPNDYCKVFEMLIGQLNSYRNYYSHAVHDYVQMDPTIIKGMQVLFD |
| | | AARREVKVRFALKTKDVDHLVRLSSRKVFGKNEAYEKETFEYRFLDASESITEKGFAFFICMWLQRKNAQEML |
| | | KKMTGFQKSDSPSQKATLETFTFYSARVPQPKLTSDNTKEGLLLDMINELKRCPKDLYPLLSNTDREKFKSIETD |
| | | KDTSFEDEYEAMPILKRSSNRFNYFALKYLDNSFEKIKFQIDLGNYCFHVYDQQIDGVVKKRRWIKKMLGFGVL |
| | | EDFADRNRPDEWKEKIQKIEGRHHKSDDVYVEETTPHYHINEQNIGLKIYDNYLESKKERKIWTPLPEFAKEDKQ |
| | | RHNETKPRNEKPDCWLSLYELPAMVFYQILFQRGYSVLSAEKIIEIHKERIERFFEDVENSRLNPGITNDALLAELE |
| | | RRNLEKSFIPKAIINYLLSKTTESFESLAEKRLNELRLETIDYLNIIKRQRIHFMDKPGSKNYIEMKSGEMANFLAH |
| | | DMIRMQKPVGKEDGKANSTEFQLLQAKLAFFTVNKSTLFETFKLCNLVDAINAHPFLFNINISECNGILDFYINYL |
| | | EQKVAYINLCKREKRYEDYHFLKIGERRRSSGKEYIGKLSKELKDDAVMNLPRGLFLKPILEALDSKDKTKRLVT |
| | | EFKKLDRINAAFIIEKYFAYVKADKPQEFYYYKRSYELLNKILDNRKDFEKRKTIAKQYFDTNELTQMTGKIEGN |
| | | PKENPITRLLPVLVRNHIFNNRIKSEDEKLRVKKVYFEKYKDFTESEKQFRLAKTCDIVLFMIADDMFRKNFVVD |
| | | KSILKNKNKVSVQDAVEIGKGYKLSEIKPNSDEGFLSLQTRVELQIQDKTIIKEQIKIKNYGDFRGLLKDRKIGSLL |
| | | PFIADKEIQFEALQKELEFYDKARIDVFAKIFEFEKSIIAKMNLQKNPEGFINHKDILKSLISENDERFILVMQLRNA |
| | | FSHSNYPDYSLFKDKVADYGFNDLKNHTSNDDTILVKSFAVQFKNLILSYYDELSRI |
| 85 | JW13189 | MRIPQLIAQNKPVFGTYATMALKNVQIVLDHIQKIESLQDQMPERRPEDFWVHPVMQFFYSERYATNVDPEKAD |
| | | AIKERLFRNFPFLKIMAENQRDYRCNKDHKGKRGYRNNNIDSSNLEINGSDIYYVLNNMFRVLKTYRDNTMHAI |
| | | IKDDKIDRNDSPFLEKNESPLSFTINNYYTVALRNVKERYSYETSDLAFIQDYRTDKRTLKTDFNFFLSMQALNG |
| | | DPTGRIHLSGVGVVQLICLFLEKKYINLFLSKLPALWGGYDESSEEARVIRRSLSINSVVLPKERIRSDKRSMSTAL |
| | | DMINELKRCPRELFDTLSQEDQDRFRVVSSDYNEVLHVRHSDRFAQLSLQYIDSNELFKGIRFHVNMGKLRYLFS |
| | | ANKSCIDGGVRVRVLEQPLNGFGRLDEMEQYRRQNDTTYGDSNIVIRDFENVQRDAACADNYPYVVDTYTRYL |
| | | LNNNKIEMCFSNDKVVPDIELDGGKWYVGKTIPHCRLSTLELPAMMFHMHLLGAEKTEQRIKSVYYNYKKLFT |
| | | GLKEGTLTKENIDSFNICRSDMPQKVLDAVSGEDKTKRVSSYVEDTLKELLEETARLIKRFEETRKAIGSSANKQ |
| | | GKRNFKSISSGKLADFLAKDIVKFQPSLEKDEYYGTDRMTGLNYRVMQSSIAMYNSMGDKKAMQDFYNMFKA |
| | | AKLVEDNKKSHPFLAVALKRIPKNAIEFYEQYLKARKQYLEKKIVEFNKTEHVRMFAANLPFINIERNKWASRD |
| | | EDFYQILGDSYLEVSPIELPRQMFDDEIKAKLKQKPEMQDIDFDKANVTYLIGEYLKRVHNDNFQEFYDWERNY |
| | | RYMDMLECKVNNKNSIEKQYLTIAEREDAWANREQGVKRYTQWANDKKLSDAKFKNMPETEYAKIVATRLSS |
| | | SRNDYQKSEKHIRRYKVQDAMMFFMVKDSLTKNINFVAKDFKLQDIMPDADKGILSGIMPMDFVFEKKGKVYT |
| | | ISSQGMKLKNYGDFFALVNDKRMSSLLELLPTPQIGKEDIEQEFKGYDDSRVDVVSVILDFEKFVYNKYPDIKNL |
| | | ALQGNHFDFLDMLKELINKGDLTENEKELLRKIRNAFSHNSYPEGGIVEIRTLPEIAKHLIRVFGNSIERIQH |
| 86 | JW13190 | MANFQEPARHIFGSYLNIARANFYKTIFHIFSISGIDCYTQKGDLYFKEDNLDKVIKVFYLISKGESTKDEGTKVSL |
| | | EEEIKASIDTVYEVIKKCYDNRWNNEVKQYTNLTEREKNARKEAFKAPLTDEGPNGEKARIRNTFTIKGEQEERF |
| | | RKMLFRHIPLLAPIMANVVAMQFKKNVNEQKESNKAMHDASLTDCLKELYSIAQCLTDCRNFYTHMNPYNSIE |
| | | KQKDQFKLQNIIAGNLDKAFAASRRIAKKRNSYSESDLTFLTGHQDDCRMQEVFVLDENGKKKWKVEKNEKGE |
| | | TKLDKEKNPIYIYKKVKAKDDKGKEKRDENGKPIYEVLYEDGKPVHEYETKLIEREDFYYRIRGIRQILNPNLTP |
| | | NGEVCDGLSAFGMLYFCSLFLSKELTVQLCTETQIFVNSPYNNDNNKKSNIILNMMFVYAIHIPRGKRLDSECSS |
| | | QALALDMLNELRRCPIELYNVLPATGKREFEDDIKHENDRTPELAKRIRSKDRFPHLALNYIDSQGLFENIRFQVR |
| | | LGSYRFCFYDKICIDGKKHPRQLHKEINGYGRLQDMEKARKEQYGHLFQLSRDESVWQKDENAYVNLRQLVPV |
| | | KAGDPAHITDTLAQYNFYQNRIGLFWNTDDETYLTDKINAQGQIVGKGLYLPPLSAVDTPTTTNRYKRKAPIDM |
| | | PAPLCSLSTYDLPALLFYEQLRRKHQLEPNDFPAAEDIIIRQYENMVRFFTEVKDMGTFDNDDALQEKLKEYGLS |
| | | KASIPQKIYRHLVQKTPDNAKDFEKSAKAELRRRLHIAEAKLSCFDKDKEKMESDKEDDRKYKKYGSKYFISIR |
| | | YAQLARYLAESIMDWTPNAKQGKDKLTGLNYRVLVSSLAGFGTRYSDEDILQILADAHIYVDEKNDHPFIGYVF |
| | | DEYPTDIEMLYSEYLKREIKEIKKYISVGIDAKTGEETITLKKDASGIPFLHHDRKRWQQSSIAELTDRYLHIKEEG |
| | | KAEPGRATLLLPDGLFAPYIVKMLEKCDSSALENCDDKSNAAFLISRYFENKCEKSQPFYDTTEPSHENDELRKV |
| | | TPYKYSRAYEYFKLLNGGKDIYLPITEIKRRLADKERRIDANIKNLTDKGNYQTIEEAREGRRRKLERAVRDMQD |
| | | NERAIRRYKTQDRILFMMAKSMLGDIVNQNADLFKLENVCENNFLSQRVKAQISVKSNNQQITIQKDDMPIKDY |
| | | GKLFRLLNDERTVQLLSYSILTNPPLIDFDQISEELKQYNTIRSSAFKSFQQMEQESFIQHERVLTDPENNDFYQGG |
| | | RRYKNNEQTKENDAKRNNFSSLLKILQCLTPEVMNMFEENDRQLITAIRNAFDHNSYPNWEVCRQLLEQEQQV |
| | | NANIQLELSQVAAFLISKIRNYVKQIEEVCHSSHQ |
| 87 | JW13191 | MPLRFKNSEERKPVLGAYAAMARNNAYIILSDIMERLHIEQPPLPDPAHPGQCLDKEAHLDKLSLFPQNNDLMPE |
| | | TKDKAMKLLCSHFPFLQTEGDLRIMNAPDGREQEKIPITYEEKCRTLLDALAVLTYLRDANLHYLFNDRRAEHL |
| | | EYRLPESKACHLLRLVLKAAPRKISERYKGTGLMPDGALDFFIKKQYVREGRSFKFNMNWLFSPQREPEKREMD |
| | | VYRKVELAGGVERDLVANPDGTPARFNRLSLFGEVLLISLFTEKRYIPDFLKACGLLTDNGFTETTQDGTLSQQR |
| | | LIREIVAAYGIRLPKRRLDITMDADQVMLDIFNELAKCPDELYLVLPEAKKKELEAVNIDGETVIKKRHSDRFVPL |
| | | LLRFIDTNELFPSIRFQVNSGYLRFLHHEKAAYMDGVERPRILQDAVNGFGRIQEVERTRSASDTYLGFPLYAPSP |
| | | DDDAPMSLPCITDSASRYIVNGECIGLSISGDRTPVIIQDGGKYRVRSPLPDCWISRYELPAMGFYLYLCKKYDLS |
| | | VSAEKIIIDKVNEYRSFFQGVANGTITSLKGITLAEKDIPGKLLDYLNGKTDSSDFNRYKKNLIQSLLEKTENRLKR |
| | | FLEDMAMVGSKENRIGKRSFVRVLPGKLASYLADDIVFFQSYPAGHPEKRLTGQQFSILQGLLATFPPDIEDSCRR |
| | | AGLLSGETAHPFLKAVFDSIPAKDRNTLVFYEIYLKRKIAFLKGPIPDSAPFLHPGKVKWQKKDAAYYKALAQR |
| | | YLDGGKDGSNTGILLPRGLFDQAITDVLTKRCPGWSRAIQNTDHANTAFKIITYLLDGQADDYQWFYYEEDRLQ |
| | | EYRFSQLIISQNAVRNGWKDLKAILQEEGVKLKGNYYKELEGRISALRRIPPERLARNTMKREELRQALRSSYDR |
| | | MCENEKSIRRIIVQDIVLFLLARWRLDPREKLNMSMIGQDESKLMNQVVTVDTLYKGQYRIRQNDVRIKDQGVL |
| | | FKTLNDKRIDSLLDQLAVGEGEVIDLEDLIAELTRYDDRRIPVVAKIMEYEKRVLKNHPELRDRSGRMDFKSVQR |
| | | ADEASSPQKKVVLRTVRNAFSHNAYPDRVVKAEGEEGIEIYNPIIPGMADGISGTTNKLVDE |
| 88 | JW13192 | MGKIVKPGEKIREMKETYKFRVNKHPFSIEEVGKNICGAYFNIARQNFYRTLLHIFSSVGIDCYTSKNSLFVKEDT |
| | | MASALEALQAIEKDKNTKEKVTQAIESLYQLVENCYDMEWELFCQKEYGGKISSINAELKKDLSEDLKNQLNTE |
| | | KEKIEKEQQDKQEEFYKDIENEKNRLRQSFILKSEQEEKLRKLLFKHLPFLSPIMADVVAYNNSHAEGDTINNGV |
| | | MKDVTLAECLQQLVTIAKCLNDCRDFYSHYNPYDSVEVQIEQFKRQNDLAIKLDKVFVASRRKVKERNQYTET |
| | | QLEFLTGKLHYRNLEKKERDNNGGKNKIPEERRYYYYRIRGERKFKDVENFEDYKDKKSPAMSDFGIVFFCSLF |
| | | LSKEYTTLMCNQLELFDHSPFTEEANNIVHNMLYVYRIHTPKGKRLDSENDENALGMDILNELRRCPMPLYEVL |
| | | PPLGRKEFEDDVRHQNSRTEEISKRLRYVDRFPSLVLKYIDAQKLFSQIRFQVRLGSYRFSFYDKICIDGEPHLRIL |
| | | HKEINGFGRLQDMEKLRKSEYGAYFPKTRQDNVWQRDENAYVDLVQLVPNKAEDDPQLTNSIAQYNIHQNRIG |
| | | LFWNQEGNSILVDKKGEDEKPLYEGYYLPPLSVVKQTTSEPEKDKNKALVEMPAPLCSLSVYDLPGLILYQHLV |
| | | ATQQGCESVDDVIIHKYQKMVSFFNDIIEGKLQPQDSVAELDKKLKDYYDLSKASVPKKVYRILSSQEFNREEDL |
| | | KAFALNALTRRLRNAIRRKTRFSEDLAKIGQKDNKYGKKNFVDVRHGALGQYLAESILDWQVTFDGGKDKLTG |
| | | LNYRVLASCLAKFGDGNTNLNYIKTILRKAKILANEGEKIANEHPFISKALDNYPDNIEDFYLDYLDAEIEYIKKY |
| | | VDIEESGQEITKVTLKSDADLSKLNFVHSERKRWVKSAAEDLKELAARYLYVEDRNVNGEVVTHRATLLLPDG |
| | | LFASPIVEVLKKRYEKDDPIMAALNNENLNHNVDYLVKLVFEKENAGDTSQPYYNSNFEVLPVYNSNTVPDFLR |
| | | ENPGNNKYVRGYELFKAIHGKPIFYSSDEIQVFLSKKITDRNTESPLKIVNRQGEECCKREIHAKIDANVEGVRTK |
| | | GDFDSLEEARNARRRKLEILLKECKESEKTIRRYKAQDMILFLMAKNLLSNAIGESDKFKLSKVCDDDFLDQKIN |
| | | AHITLHLDDYTVKIQKEEMPIKNYGQLFRLLSDNRVMTILSYPSINEATLEYDDLIEELSIYDERRTSVFDVAQKL |
| | | ERASFVQHKEELTNPENDLFYCHNKRYRVNSGRENQPKRNNFEAMLKLLLKMTPQQQSMFSKVEQDFIKNIRN |
| | | SFGHNGYPIRNTIERIANGNDPKFADLKEMQLKGIASLFAGKLLDCLNDNNIE |
| 89 | JW13193 | MKLSLKPDSQLKPIFGAYAAMARNNIFLTVLDVMKQLHIPQEPLFDPRGNELDLEANIWKLSLFVIHLKPEQSSK |
| | | ADKLLRTHFPFLDLLNDIKIGSIKGESFHYNVSFKDTKQLLLNGVNVLSHLRDINLHYKTNDGKINDYYYRKYEN |
| | | VWSNHLIGLLKAAPRVIKERYKQTSLLSPQSMDFFTKDSYLKNTDGYILNPDWLFCPTRKPGDNELYIYKDKKP |
| | | YRVEKERFDRLTRFGEILTIALFIERRYISDFLSECGLLGQFPSSTNDGSISQRRLLKELISVYCIHLPNRRLDISIGET |
| | | QIKLDMLNELAKCPDELYMVLPDTIKTKFEIIGIDGRPVFMKRYSDRYPALLLNYFDTTGSFEKLRFQVNAGYLK |
| | | YVHHGITDYMDGVGRVRIIQDGLNCFGRRHEVESKLKEQFSIHGFPVIRNNEEEANIDYPFITESGTRYVFNGDMI |
| | | GISLYGDIMPRFEQYTENNTVRYRVRNTTPDCWISKYELPAMAFYYYLSKKYNEGVSVEQLLSQTTEQYRKFFQ |
| | | GVSDGTIKSLSDIDIPIKAIPQKLQLFLKGESSHEDYNKFKERVISKMIEETDIRLSRLKDNLEVVFQRDNKIGKKT |
| | | RVRLKPGVLSDYLAKDIVRFQSFPDNHPEKKMTGQQFSILQGLIATFSEELEVSCKRAGLLDGPSSHPFLSRVFSR |
| | | HRGQMRSTIDFYQAYLEERRAYLSGSIPDDAHFLHPNRVKWSHDKQESYFRSLSTRYIKDEITGERIGIFLPRGLF |
| | | LKPALKIIANNCPKTARFLKEQERINMSFLILTYLEEELQDQNQGYYYDHLETGNGEYDFYKIIDREKKEFGDKR |
| | | LGYIIRAERDSCQNSLYFFQLKNAFGHVRESARPIAGTKELAEKLRAAYNKMCKNEKKIRQYMIQDATLFLLSRD |
| | | VVGPAGGQMKLADIDPTGNSVLEQTVDVTTNYKGKTIRQRNVKVKNYGELYSVLSDKRINTLLEYQPKTENSIP |
| | | LEAIKEELVSYDHERVPVITKIQGYERAICEEKPELFSDEDARYDFNDIQNADTKTPNLNKVALKKIRNAFSHNQY |
| | | PAPVVKQGNKEIVIFTHSLPDVAREISSKAGELTNWTPEEGSE |
| 90 | JW13194 | MARTNFVKTINYILPIAGVRGNYSESQINKMLHALFLIHAGRSSELSVEQSNWKKRLTLNVEQQARLQRLLFKHF |
| | | PVLQPMMADAAEHKAYLAKNKKKSDVQTDDDVFAQFKGVSLAECLNVVALMADTLTECRNFYTHKDPYNTP |
| | | EQLEKQYEHQEFIALKLDKVVVASRRVLKDREGLTVGEVEFLTGIDHMKQVDKKDEKGNVIYVEDRGTRKKA |
| | | KAFVEYDDFYFKITGSRTAGGKACSALSDFGLLYFCILFLSKPYAKLFIDESRLFEFSPFDENENIIMQEMLSIYRIR |
| | | TPRLHRIDSRDNKAALAMDIFGEMRRCPIELYDLLDKEHGQPFFHDEVKHPNSHTPEVSKRLRLNDRFPHLALRY |
| | | IDEMELFKRVRFQLQMGAFRYKFYDKECIDGRVRVRRIQKDINGYGRLQDVADKRMERWDDLIQKREERPVKL |
| | | EHEELYIDLDQFTQDSADSTPYVTDRRPAYNIHANRIGMFWEESQNPQGFKFFDKNGMYIPVLSVTDDHKAPIA |
| | | MPAPRCTLSVHDLPAMLFYEYLLSQLDQEARKQYHSAEQIIIGIEADYRKFFGAVADGNIKPFASSEKFADYLEE |
| | | NYPSMLLSDIPQKIRQYLSSEHLSYEGKPVTVRERLVEVTLKHLAERMERLERRLKNYTEDLKKIGNKDNKIGKK |
| | | DFADVRHGALARYLVQDMMEWQPSKDGKGADKLTGLNYNVLTAFLATYGTPQASIADSDIAKLSLKEVLRNA |
| | | HLVGGDNPHPFLEEVLAYDNKNIEDFYLNYLTEEVNHLNSRIQSLSSNPTYKALESLPFVHHNRIRYNERTDEEIR |
| | | ALAGRYATIQLPDGLFIPNIVDLLENQYATNTDMQKALSFEGADHLNPTRNATYLISQFYKTVLHDNAQPFYQT |
| | | DKSYTRQQDGQPPVAFSFRRAYDLFTELNNDKEDYFPFELKPLFLTSEEIQQRLTEKRLGKDGNPIPQRENGEIKK |
| | | DAQGNIIWERKITHDVDIFVNTLSNKDLKCSKNEVKEKRDAKREKMKRMVHEIKNNERTIRRYRTQDMILFLLA |
| | | KKMFVDLLGEQSRSVDWKQLRLSKVCNDAFLRQTLTFRVPVTVGDNTVYVEQENMSLKNYGEFYRFLTDDRL |
| | | TSLFENIVDSIEPDKKTGERVISHTDLMSELAAYDQRRSTVFRHIQQIEDIVISGNAILANPDCDNFWVDGKRGEL |
| | | AKRNNFASLLKMIDLINTTSLTDDERTLLVAIRNAFGHNSYRVDLKKIGGINNLPEVANSILKHLETLSGFSAE |
| 91 | JW13195 | MSKNDNIKSKAKALGLKSTFQVGDEVVMTSFGKGNKAIVEKIVRGTDVQSVPTEPNFSAEIEGKKFDLVGRAHI |
| | | QTKSDNPQYSKKRTGDDMIGAKAALEKRFFGGTFDDNIHIQLAYNVLDIEKILSVHINNIVYTLNNIRRKDNAED |
| | | DDFIGYMSTRNDYDTFIEPRKHNISEDAAKSIDKSRASFEEYLQPPVKDCLHYFGNTFFAPREIEKTYTDNRGFER |
| | | KKKVTVNSLIDEKEIYYIFALLGGLRQFCTHDNKSGRNWLYSLEENGINSDAKAVLDKYYNSAVARIDESFVDN |
| | | ASKTNFKLIFNAMDVSDEALQDNIAKAFYKFTVCKSFKNMGFSIKKLREQLLELPEYEGLKDKHYDSVRSKLYQ |
| | | IMDFVIYLSFKDEKFKKDNEEKINNIVNELRATLNEEDKGRVYASYAERMKSELKPAIARLKSDIDKIKDSRVKEF |
| | | ELDASVKYRLSKVVESVRLKDRATYFTKLIYLTTLFLDGKEINDLLTTLIHQFENIASFIDVMNDRGIDCRFSDGY |
| | | KLFESSKQIAFELRNVNSFARMTRTSKDDENATHMMYIDAAEILGTDYTEEQIEEHLNLEKKRMIPGTKKADMN |
| | | FRNFIINNVIKSSRFNYLVRYSNPKKIRALADNEGVIRFVLGELPDAQIDRYTLLCGFNPDADRQEKTDKLAKAIT |
| | | GLRFNDFENVKQGANTEGESQESIDKAQKQGLISLYLTVLYLLTKNLVYVNSRYFLAFHCLERDAQLLGSGAGH |
| | | HEPYVALTQRFINEDKLNEHACEYLKTNIANSDEYTIRIFRNNAAHLSAVRNANLYIDKLKEFKSYYEIYHFLSQE |
| | | NIYGKYCVDKKYVTADENGSKTISVKISKDYCPQVYIDKSLEYFDKLNKYGTYCKDFTKALNSPFGYNLARYKN |
| | | LSIEGLFDRNRPGDKGENTFED |
| 92 | JW13196 | MNFEENGNKYLFGAFLNTAYDNYNEVISHIYDVIRRITNNNPTEQKSSSQALASEEPISSEEKEKMFFVCTILIAGN |
| | | ATSRKIESILKRAFPFWDDFIEIHASLSAQLDKTESIFQLQKNCVEELFTALESWRNYTTHYIHPEAIASPTINLLLK |
| | | SCFNQGIKEIKKRFNTKDEQLTHLYEPELFHNKETNKDEIKCYHPANYYRFANENGLLTEKGLAFFTCLFLYKRE |
| | | SYLLLNQLKGFKRADEERYRMTLEAFTVMRFRAPKQLLKLQQQQLDKVSLGISMINELAKCPKELYDCLENEK |
| | | RRQFEVYPDTETTLYEQEAIGELIRYKDRYENLMLKALEHSSECKYMGFYLSLGKCYMKCYEKHYIDGTTDNR |
| | | YIVHSLYGFGRLQNASTDHKNHLSSFGEELQTHSQLPVCHFATEDDAADSPTSVPYISESYPSYVLNNNNIGIKW |
| | | LNSSEKTEYPNFNLNEGKVSCPAPDYWLSKYELPAMAFYAYLKEKHKNKLQEYPSIKELIDQSHPEATKTSSSTI |
| | | KDRLEERLKRYKVDATKKLKRLAEKPDGVQSGRLADELARDILWLQPSSPKEGGKDKLTGANFQALQYALAR |
| | | YDFMRTELPRILTAARLTGSNNAHPFLDKINPQDYSRLTAYYAGYLTQKIVYIDKQLEKLARGKVNIQFHPTRKL |
| | | IRQSQTDSVEHEKETVFLPRNIFTEKIEQALCQIQGNFKLNIEKARISGKKLNASKLILIYLELYLKDEVPEFYNAK |
| | | RKYPCLCYPLTPSEKYTLKEYQDTEERSKRIDRLKCFYNFKEVAEKVYLHKELSEDENKRYRKYIEVQKVIDNE |
| | | KIIRLRKVQDITLYLWTRLFIEENPEQIFFSKNEKPENSLFKLREMNNAILDNTTSRKININTPNGNVSIEDEFKIKD |
| | | YGRVTALTTEKLPQSLIKFIAILNKPLNKKDRPLLSYDYIYHETNAFYRYRPEIIKMAQDIENKIINDLKINVKPQA |
| | | ESYYNFRRTIQKLENKSRDINIELLIKIRNAFCHNEYKELDNDQYKYLNSLFKLSSDIKQTDTLAYLLYILFETEYQ |
| | | KVMILK |
| 93 | JW13197 | MANTQFKLKSVDYSKPYDEQPECKRHVVGAYANLARHNMTLTVNTIMQAIGMEIFDENNIGDAFNSSHRKKID |
| | | ALDNIQKVNLQKRLYRHFPFFKRMKLEDEDKKSVQLTTLLEVMSDFTSCMAIIRNFYTHYHPYNSPDEKKRQLE |
| | | LKKRMGKRLQFLYENTSQMFKTTEALDHESNEVLSALRIPEDYIEEILPGDKEYDNLFNLINDPRTSKFKKQGLK |
| | | LNQQTKVITRKKVRFVRNPDYQAYMIDETNGMSDVAIIFFLCLFLDKKVSFDMMDEVGFTKQIKFNGPQADRQL |
| | | LYLKEIMCMNRIRMTKAKLDSEMTDTALALDMLGEVRKCPKALYEVLDKDARNEFKDDATVQWETEHGKEA |
| | | VSTEALASESDEDELTDKNTPRSTFVRWDDRFPQMALRYIDYQGLENDIRFQLNLGKYRFAFYQHDNKQSVDNE |
| | | ERLRILQKELHGFGRIQEVEEKKNEKWKELYDEKTVKDGLTQKKPDEAGQAPYITEQYAQYAIDEKSHSIGLRW |
| | | EGWDKKDKSKGHYGDLDKLKMFIPYIPSKPKDEEQQTNQAEDLMAPQAMLNLYELPALLFYQYLLDKYKSDT |
| | | SDAEDIIKTSFTNLVNFFNDVESDVLVPIDDPDENERIDKLSSLLEGYNLQMSDIPEKLRNYLCNKDIDYDLKMEK |
| | | SAIGRLEEKKKQVDKALSSYKDKKKIIGSKDNKFDKMRASIKTGQLATWLIRDIMDWVPNNSSARNRLTGQNYT |
| | | ALQATMALFGQQANSNRSYLESCFRSAHLLPLDGKPYDKNTHHPFLEDVIQAMEDETLESFYEKYLDFETKHID |
| | | KVIKTLENGTDFDDYYKVPFLHCDRQRWAGPDKSLAAQYLKRPIQLPNGIFTKPVFNLLSTIDNKALRKALEESQ |
| | | QANGENPPLANNVAFLMKLYFEFVEHDHSQPFYYTTTSDGNKSHYLHIYRIFKKLYGQPIPHSNKTTVPAYTIDEI |
| | | RHLRKQAIADIEEYVDNEIEKWKRTKQDRFESKLRKKLKKENDKRYKKHEKQLNINEEIQKVVKKEIDTMRTD |
| | | MTSKMKRQLRSVYDNERTIRRFKAQDMMLLVMARNILKAKSNDKDFTDGFCLENVFSDSLLDKPIDFEWKVNI |
| | | KGASKTIKQEGMKMKNYGQFYKFASDHQRLESLLSRLPDSLFQRAEIENELAYYDSNRSKVFRQVYIIESEAYRL |
| | | KPGLQDDANASKDWFYFTDKKTHKQRSIRNSFIQLLEILAAGEDGILDNDEKIALQKTRNAFGHNTYDVDLNVV |
| | | FKGKENKKKIPEVANGIKEKIETQTEELKRRSK |

TABLE B
gRNA sequences

| SEQ | | |
|---|---|---|
| ID NO: | Name | Sequence |
| 94 | JW13101_Guide1_3′ | GACUGAACCUUACCAUGUAGGGAAUUGAACUCCUAAAAUUCCCUUAGUCAGAG |
| 95 | JW13102_Guide1_3′ | GUUGUAGAAGCUCUUAUUUUGAAGGGUAUAACAACUUCCUAAAAUUCCCUUAGUCAGAG |
| 96 | JW13103_Guide1_3′ | AUGCCUUAAUUUUGGAAGGUGAGAACAACUCCUAAAAUUCCCUUAGUCAGAG |
| 97 | JW13104_Guide1_3′ | GUUGUAACUGCCCUUAUUUUGAAGGGUAUAAACAACUCCUAAAAUUCCCUUAGUCAGAG |
| 98 | JW13105_Guide1_3′ | GCUGUAGAAGCCUCCGAUUUGUGAGGUGAUGACAGCUCCUAAAAUUCCCUUAGUCAGAG |
| 99 | JW13106_Guide1_3′ | UAGUUUUAUUACCCCUACAAACAGGGUUAUAUUCUAACUCCUAAAAUUCCCUUAGUCAGAG |
| 100 | JW13107_Guide1_3′ | GUUGUAACUGCCCUUAUUUUGAUGGGUAAAAACAACUCCUAAAAUUCCCUUAGUCAGAG |
| 101 | JW13108_Guide1_3′ | GUUGUGCUUACCCUACAAAAUUAUCCUAAAAUUCCCUUAGUCAGAG |
| 102 | JW13110_Guide1_3′ | GUUGUUGAAUGCUGCAUUUUGGGUAGCAAUUACAACUCCUAAAAUUCCCUUAGUCAGAG |
| 103 | JW13112_Guide1_3′ | AGUCCCCGCUCGUUUCGGGGUGGUCUUCCUAAAAUUCCCUUAGUCAGAG |
| 104 | JW13113_Guide1_3′ | GUUGUUUUUAUUCUUCAAAAUAAGAGCAGUUACAAUCCUAAAAUUCCCUUAGUCAGAG |
| 105 | JW13114_Guide1_3′ | GUUGUCUUUACCCUCCAAACCAAGAGCAUUCACAACUCCUAAAAUUCCCUUAGUCAGAG |
| 106 | JW13115_Guide1_3′ | GUUGUGGGUAUUCUGCAAAUUUAGGGCAAUAACAAAUCCUAAAAUUCCCUUAGUCAGAG |
| 107 | JW13116_Guide1_3′ | CGAAUUCCGAAUUAACCUCGUGUAGUGACGUCGUUCUCCUAAAAUUCCCUUAGUCAGAG |
| 108 | JW13117_Guide1_3′ | GUUGUUUUCACCUUGCAAAUAUAAGGCAUUUACAACUCCUAAAAUUCCCUUAGUCAGAG |
| 109 | JW13118_Guide1_3′ | GUUGUAACUGCUCUUGUUUUGAAGGGUAAACACAACUCCUAAAAUUCCCUUAGUCAGAG |
| 110 | JW13119_Guide1_3′ | UGCUCUCAGUUUUGGAGGGUAAACACAACUCCUAAAAUUCCCUUAGUCAGAG |
| 111 | JW13120_Guide1_3′ | AUGCCUUAAUUUUGGAAGGUGAAAACAACUCCUAAAAUUCCCUUAGUCAGAG |
| 112 | JW13121_Guide1_3′ | GCUGUACCUACCCUGCAAAUCAGGAGCCUCUACAGCUCCUAAAAUUCCCUUAGUCAGAG |
| 113 | JW13122_Guide1_3′ | AAGUUGUCAUACCCCUCCCAACGAGGGGCUUCUACAACUCCUAAAAUUCCCUUAGUCAGAG |
| 114 | JW13123_Guide1_3′ | GUUGUAGCAACCUAUAUUUUGUUAGGAAAAGACAACUCCUAAAAUUCCCUUAGUCAGAG |
| 115 | JW13124_Guide1_3′ | GGACUACACCCUUACCGUUGUGUAGGGGUCUAAAACUCCUAAAAUUCCCUUAGUCAGAG |
| 116 | JW13125_Guide1_3′ | GUUCAGCAUGACCGCUUCUUUUACAGCGGACUGAACCUCCUAAAAUUCCCUUAGUCAGAG |
| 117 | JW13101_Guide1_5′ | UCCUAAAAUUCCCUUAGUCAGAGGACUGAACCUUACCAUGUAGGGAAUUGAAC |
| 118 | JW13102_Guide1_5′ | UCCUAAAAUUCCCUUAGUCAGAGGUUGUAGAAGCUCUUAUUUUGAAGGGUAUAACAACU |
| 119 | JW13103_Guide1_5′ | UCCUAAAAUUCCCUUAGUCAGAGAUGCCUUAAUUUUGGAAGGUGAGAACAAC |
| 120 | JW13104_Guide1_5′ | UCCUAAAAUUCCCUUAGUCAGAGGUUGUAACUGCCCUUAUUUUGAAGGGUAUAAACAAC |
| 121 | JW13105_Guide1_5′ | UCCUAAAAUUCCCUUAGUCAGAGGCUGUAGAAGCCUCCGAUUUGUGAGGUGAUGACAGC |
| 122 | JW13106_Guide1_5′ | UCCUAAAAUUCCCUUAGUCAGAGUAGUUUUAUUACCCCUACAAACAGGGUUAUAUUCUAAC |
| 123 | JW13107_Guide1_5′ | UCCUAAAAUUCCCUUAGUCAGAGGUUGUAACUGCCCUUAUUUUGAUGGGUAAAAACAAC |
| 124 | JW13108_Guide1_5′ | UCCUAAAAUUCCCUUAGUCAGAGGUUGUGCUUACCCUACAAAAUUA |
| 125 | JW13110_Guide1_5′ | UCCUAAAAUUCCCUUAGUCAGAGGUUGUUGAAUGCUGCAUUUUGGGUAGCAAUUACAAC |
| 126 | JW13112_Guide1_5′ | UCCUAAAAUUCCCUUAGUCAGAGAGUCCCCGCUCGUUUCGGGGUGGUCU |
| 127 | JW13113_Guide1_5′ | UCCUAAAAUUCCCUUAGUCAGAGGUUGUUUUUAUUCUUCAAAAUAAGAGCAGUUACAA |
| 128 | JW13114_Guide1_5′ | UCCUAAAAUUCCCUUAGUCAGAGGUUGUCUUUACCCUCCAAACCAAGAGCAUUCACAAC |
| 129 | JW13115_Guide1_5′ | UCCUAAAAUUCCCUUAGUCAGAGGUUGUGGGUAUUCUGCAAAUUUAGGGCAAUAACAAA |
| 130 | JW13116_Guide1_5′ | UCCUAAAAUUCCCUUAGUCAGAGCGAAUUCCGAAUUAACCUCGUGUAGUGACGUCGUUC |
| 131 | JW13117_Guide1_5′ | UCCUAAAAUUCCCUUAGUCAGAGGUUGUUUUCACCUUGCAAAUAUAAGGCAUUUACAAC |
| 132 | JW13118_Guide1_5′ | UCCUAAAAUUCCCUUAGUCAGAGGUUGUAACUGCUCUUGUUUUGAAGGGUAAACACAAC |
| 133 | JW13119_Guide1_5′ | UCCUAAAAUUCCCUUAGUCAGAGUGCUCUCAGUUUUGGAGGGUAAACACAAC |
| 134 | JW13120_Guide1_5′ | UCCUAAAAUUCCCUUAGUCAGAGAUGCCUUAAUUUUGGAAGGUGAAAACAAC |
| 135 | JW13121_Guide1_5′ | UCCUAAAAUUCCCUUAGUCAGAGGCUGUACCUACCCUGCAAAUCAGGAGCCUCUACAGC |
| 136 | JW13122_Guide1_5′ | UCCUAAAAUUCCCUUAGUCAGAGAAGUUGUCAUACCCCUCCCAACGAGGGGCUUCUACAAC |
| 137 | JW13123_Guide1_5′ | UCCUAAAAUUCCCUUAGUCAGAGGUUGUAGCAACCUAUAUUUUGUUAGGAAAAGACAAC |
| 138 | JW13124_Guide1_5′ | UCCUAAAAUUCCCUUAGUCAGAGGGACUACACCCUUACCGUUGUGUAGGGGUCUAAAAC |
| 139 | JW13125_Guide1_5′ | UCCUAAAAUUCCCUUAGUCAGAGGUUCAGCAUGACCGCUUCUUUUACAGCGGACUGAACC |
| 140 | JW13126_Guide1_3′ | GUUGUAACUGCUCUUGUUUUGAAGGGUAAACACAACUCCUAAAAUUCCCUUAGUCAGAG |
| 141 | JW13127_Guide1_3′ | UUCAAAAUAAGGGCAGUUACAACUCCUAAAAUUCCCUUAGUCAGAG |
| 142 | JW13128_Guide1_3′ | GUCUAAGACUAGGUAAGAAUUAGCGGUGUCCAUUGCUCCUAAAAUUCCCUUAGUCAGAG |
| 143 | JW13129_Guide1_3′ | GCUGGAGCAGCCCUCGAUUUGCAGGGUAAUCACAGCUCCUAAAAUUCCCUUAGUCAGAG |
| 144 | JW13130_Guide1 3′ | GUUGUAACUGCCCUUAUUUUGAAGGGUAUAAACAACUCCUAAAAUUCCCUUAGUCAGAG |
| 145 | JW13131_Guide1_3′ | GUUGUGUGUAACCUAAAAAAUGAGGGCAAUAACAACUCCUAAAAUUCCCUUAGUCAGAG |
| 146 | JW13132_Guide1_3′ | GCUGUAGAAGCCUCCGAUUUGUGAGGUGAUGACAGCUCCUAAAAUUCCCUUAGUCAGAG |
| 147 | JW13133_Guide1_3′ | GUUGUAACUGCCCUUAUUUUGAUGGGUAAAAACAACUCCUAAAAUUCCCUUAGUCAGAG |
| 148 | JW13134_Guide1_3′ | GCUGGAGCAGCCCUCGAUUUGCAGGGUAAUCACAGCUCCUAAAAUUCCCUUAGUCAGAG |
| 149 | JW13135_Guide1_3′ | GUUGUUCUCACCUUGCAAAUAAUAAGGCAUAUACAUCCUAAAAUUCCCUUAGUCAGAG |
| 150 | JW13136_Guide1_3′ | GGUGUACCUGCCAUGCAAAUAGAUGGCUUUCACACCUCCUAAAAUUCCCUUAGUCAGAG |
| 151 | JW13137_Guide1_3′ | GCUGUAAUCACCCCGCAAAACCAGGGUUGCUUCAGCUCCUAAAAUUCCCUUAGUCAGAG |
| 152 | JW13138_Guide1_3′ | GUUGUCUUUACUCCACAAACUAAGAGCAUUCACAACUCCUAAAAUUCCCUUAGUCAGAG |
| 153 | JW13139_Guide1_3′ | GGUGUUUUAAUCUUUCCAACUAAAGACUUUUACACCUCCUAAAAUUCCCUUAGUCAGAG |
| 154 | JW13140_Guide1_3′ | CCUUCUUUUUGAAAGGCACAAAUACCUCCUAAAAUUCCCUUAGUCAGAG |
| 155 | JW13141_Guide1_3′ | GUUGUUUUCACCUUGCAAAUAUAAGGCAUUUACAACUCCUAAAAUUCCCUUAGUCAGAG |
| 156 | JW13142_Guide1_3′ | GUUGUUUUUACCCUUCAGAAUAAGAGCAGUUCCUAAAAUUCCCUUAGUCAGAG |
| 157 | JW13143_Guide1_3′ | ACGUUGUCAUACCCCUCCUAACGAGGGGCUUCUACAACUCCUAAAAUUCCCUUAGUCAGAG |
| 158 | JW13144_Guide1_3′ | GUUGUGAAUGCCAUAUUUUUGAAAGGUAGAAACAACUCCUAAAAUUCCCUUAGUCAGAG |
| 159 | JW13145_Guide1_3′ | GUUGUAGCAACCUAUAUUUUGUUAGGAAAAGACAACUCCUAAAAUUCCCUUAGUCAGAG |
| 160 | JW13146_Guide1_3′ | GUUGUUGAAUGCUGCAUUUUGGGUAGCAAUUACAACUCCUAAAAUUCCCUUAGUCAGAG |
| 161 | JW13147_Guide1_3′ | GCUGUACCUACCCUGCAAAUCAGGAGCCUCUACAGCUCCUAAAAUUCCCUUAGUCAGAG |
| 162 | JW13148_Guide1_3′ | UAGUUUUAUUACCCCUACAAACAGGGUUAUAUUCUAACUCCUAAAAUUCCCUUAGUCAGAG |
| 163 | JW13149_Guide1_3′ | UGCUCUCAGUUUUGGAGGGUAAACACAACUCCUAAAAUUCCCUUAGUCAGAG |
| 164 | JW13126_Guide1_5′ | UCCUAAAAUUCCCUUAGUCAGAGGUUGUAACUGCUCUUGUUUUGAAGGGUAAACACAAC |
| 165 | JW13127_Guide1_5′ | UCCUAAAAUUCCCUUAGUCAGAGUUCAAAAUAAGGGCAGUUACAAC |
| 166 | JW13128_Guide1_5′ | UCCUAAAAUUCCCUUAGUCAGAGGUCUAAGACUAGGUAAGAAUUAGCGGUGUCCAUUGC |
| 167 | JW13129_Guide1_5′ | UCCUAAAAUUCCCUUAGUCAGAGGCUGGAGCAGCCCUCGAUUUGCAGGGUAAUCACAGC |
| 168 | JW13130_Guide1_5′ | UCCUAAAAUUCCCUUAGUCAGAGGUUGUAACUGCCCUUAUUUUGAAGGGUAUAAACAAC |
| 169 | JW13131_Guide1_5′ | UCCUAAAAUUCCCUUAGUCAGAGGUUGUGUGUAACCUAAAAAAUGAGGGCAAUAACAAC |
| 170 | JW13132_Guide1_5′ | UCCUAAAAUUCCCUUAGUCAGAGGCUGUAGAAGCCUCCGAUUUGUGAGGUGAUGACAGC |
| 171 | JW13133_Guide1_5′ | UCCUAAAAUUCCCUUAGUCAGAGGUUGUAACUGCCCUUAUUUUGAUGGGUAAAAACAAC |
| 172 | JW13134_Guide1_5′ | UCCUAAAAUUCCCUUAGUCAGAGGCUGGAGCAGCCCUCGAUUUGCAGGGUAAUCACAGC |
| 173 | JW13135_Guide1_5′ | UCCUAAAAUUCCCUUAGUCAGAGGUUGUUCUCACCUUGCAAAUAAUAAGGCAUAUACA |
| 174 | JW13136_Guide1_5′ | UCCUAAAAUUCCCUUAGUCAGAGGGUGUACCUGCCAUGCAAAUAGAUGGCUUUCACACC |
| 175 | JW13137_Guide1_5′ | UCCUAAAAUUCCCUUAGUCAGAGGCUGUAAUCACCCCGCAAAACCAGGGUUGCUUCAGC |
| 176 | JW13138_Guide1_5′ | UCCUAAAAUUCCCUUAGUCAGAGGUUGUCUUUACUCCACAAACUAAGAGCAUUCACAAC |
| 177 | JW13139_Guide1_5′ | UCCUAAAAUUCCCUUAGUCAGAGGGUGUUUUAAUCUUUCCAACUAAAGACUUUUACACC |
| 178 | JW13140_Guide1_5′ | UCCUAAAAUUCCCUUAGUCAGAGCCUUCUUUUUGAAAGGCACAAAUACC |
| 179 | JW13141_Guide1_5′ | UCCUAAAAUUCCCUUAGUCAGAGGUUGUUUUCACCUUGCAAAUAUAAGGCAUUUACAAC |
| 180 | JW13142_Guide1_5′ | UCCUAAAAUUCCCUUAGUCAGAGGUUGUUUUUACCCUUCAGAAUAAGAGCAGU |
| 181 | JW13143_Guide1_5′ | UCCUAAAAUUCCCUUAGUCAGAGACGUUGUCAUACCCCUCCUAACGAGGGGCUUCUACAAC |
| 182 | JW13144_Guide1_5′ | UCCUAAAAUUCCCUUAGUCAGAGGUUGUGAAUGCCAUAUUUUUGAAAGGUAGAAACAAC |
| 183 | JW13145_Guide1_5′ | UCCUAAAAUUCCCUUAGUCAGAGGUUGUAGCAACCUAUAUUUUGUUAGGAAAAGACAAC |
| 184 | JW13146_Guide1_5′ | UCCUAAAAUUCCCUUAGUCAGAGGUUGUUGAAUGCUGCAUUUUGGGUAGCAAUUACAAC |
| 185 | JW13147_Guide1_5′ | UCCUAAAAUUCCCUUAGUCAGAGGCUGUACCUACCCUGCAAAUCAGGAGCCUCUACAGC |
| 186 | JW13148_Guide1_5′ | UCCUAAAAUUCCCUUAGUCAGAGUAGUUUUAUUACCCCUACAAACAGGGUUAUAUUCUAAC |
| 187 | JW13149_Guide1_5′ | UCCUAAAAUUCCCUUAGUCAGAGUGCUCUCAGUUUUGGAGGGUAAACACAAC |
| 188 | JW13150_Guide1_3′ | GUCAUAGACCCCUUCAAUUUAUGAGGGGUUUUGUUCUCCUAAAAUUCCCUUAGUCAGAG |
| 189 | JW13153_Guide1_3′ | GUUGUGGAAGCCCUCAUUUUGAGAGGGUAUUACAACUCCUAAAAUUCCCUUAGUCAGAG |
| 190 | JW13154_Guide1_3′ | GUUGUGCUUACCCUUAAAACUAAGGGCAGCUACAACUCCUAAAAUUCCCUUAGUCAGAG |
| 191 | JW13155_Guide1_3′ | GUUGUAAGUGACCUCAUUUUGGAAGGUAAAAACAACUCCUAAAAUUCCCUUAGUCAGAG |
| 192 | JW13156_Guide1_3′ | GUUGUGUAUAUCUCCCAAAUCAGAGACAGCUACAACUCCUAAAAUUCCCUUAGUCAGAG |
| 193 | JW13157_Guide1_3′ | GUUGUUUAUACCUUACAAAAUAAAGGCUCUCACAACUCCUAAAAUUCCCUUAGUCAGAG |
| 194 | JW13158_Guide1_3′ | CCUUUGGAGUACUGUUAUUUAACAGGGUUCUAAAACUCCUAAAAUUCCCUUAGUCAGAG |
| 195 | JW13159_Guide1_3′ | GUUGUCGAAAGCCUUAUUUUGAAGGGCUAUUACAACUCCUAAAAUUCCCUUAGUCAGAG |
| 196 | JW13160_Guide1_3′ | AUGACUAUACCAGCAAUGGCUGGAUUAAAACUCCUAAAAUUCCCUUAGUCAGAG |
| 197 | JW13161_Guide1_3′ | GCUGUAGAGGCCCCCGUUUUAGGGGGUAGGUACAGCUCCUAAAAUUCCCUUAGUCAGAG |
| 198 | JW13162_Guide1_3′ | GCUGUUACUGCCCCACAAAUCGAGGCCUAUCACAGCUCCUAAAAUUCCCUUAGUCAGAG |
| 199 | JW13163_Guide1_3′ | GCUGUAGAGGCCCCCGAUUUUCCUAAAAUUCCCUUAGUCAGAG |
| 200 | JW13164_Guide1_3′ | GUUGUGCCUACCUCUCAAAUAUAGGGCUUUCACAAUCCUAAAAUUCCCUUAGUCAGAG |
| 201 | JW13165_Guide1_3′ | GUUGUAAAUACCCUACAAAUUCCUAAAAUUCCCUUAGUCAGAG |
| 202 | JW13166_Guide1_3′ | GAACUAUAGUAGUGUAAAUUUACACUACUCUAAAACUCCUAAAAUUCCCUUAGUCAGAG |
| 203 | JW13167_Guide1_3′ | GCUGUUAUAUCCUUACCUUUGUAAGGGAAGUACAGCUCCUAAAAUUCCCUUAGUCAGAG |
| 204 | JW13168_Guide1_3′ | GUUGUAGUUCCCUUUUAUUUAGGGAUGGUGUUCAGCUCCUAAAAUUCCCUUAGUCAGAG |
| 205 | JW13169_Guide1_3′ | GUUGUAAAAUCUCUCGUUUUGGAGAGGAUUAACAACUCCUAAAAUUCCCUUAGUCAGAG |
| 206 | JW13170_Guide1_3′ | GUUGUAGAAGCCUGUCGUUUGGAUGGGUAUUCCUAAAAUUCCCUUAGUCAGAG |
| 207 | JW13171_Guide1_3′ | GUUGUCAUACCUACCCAAACGAUAGGCUUCUACAACUUCCUAAAAUUCCCUUAGUCAGAG |
| 208 | JW13172_Guide1_3′ | CUUAUACAACACCCAUUUUCACAGUGGGUCUAUAACUCCUAAAAUUCCCUUAGUCAGAG |
| 209 | JW13173_Guide1_3′ | GUUGUCAUACCGCUCCGAAUGAACGGCUUCUACAACUCCUAAAAUUCCCUUAGUCAGAG |
| 210 | JW13174_Guide1_3′ | GUUGUCAUACCGCCCCGAAUGAGCGGCUUCUACAAUUCCUAAAAUUCCCUUAGUCAGAG |
| 211 | JW13175_Guide1_3′ | GCUGUCAUUACCCUCCAAAACGAGGGCUUCUACAGCUCCUAAAAUUCCCUUAGUCAGAG |
| 212 | JW13176_Guide1_3′ | GUUUCAGAACCCCGUAAUUCUACGGGGUUGUAGUUGUCCUAAAAUUCCCUUAGUCAGAG |
| 213 | JW13177_Guide1_3′ | GUUUUAGACCCCUGUGAUUCAGGGGUGUAGUGAAACUUCCUAAAAUUCCCUUAGUCAGAG |
| 214 | JW13178_Guide1_3′ | GUUUUAAGACUGUGCAAAUUCGCAUAGCUUUCAAUCUCCUAAAAUUCCCUUAGUCAGAG |
| 215 | JW13179_Guide1_3′ | GUUGUAGAAGCCUGUCGUUUGGAUGGGUAUAACAACUCCUAAAAUUCCCUUAGUCAGAG |
| 216 | JW13180_Guide1_3′ | GUUGUCAUACCCACUCAAAUUGUGGGCUUCUACAACUCCUAAAAUUCCCUUAGUCAGAG |
| 217 | JW13181_Guide1_3′ | GUUUCCAUACCCACUCAAAUGCGGGCUUCUGAAACUCCUAAAAUUCCCUUAGUCAGAG |
| 218 | JW13182_Guide1_3′ | GUUGUGUGUGCCUUUCAAAUUGAAGGCGUUCCCAACUCCUAAAAUUCCCUUAGUCAGAG |
| 219 | JW13183_Guide1_3′ | GCUGUACUUACCAUACAAAUUUAUGGCUUUUACAGCUCCUAAAAUUCCCUUAGUCAGAG |
| 220 | JW13184_Guide1_3′ | GUGCGAGACCAUCCCCAAAUGAAGGGGAACUACACCUCCUAAAAUUCCCUUAGUCAGAG |
| 221 | JW13185_Guide1_3′ | GGUGUAGUUCCCAUUUAGUUGGGGAUGUUCUCCCGCUCCUAAAAUUCCCUUAGUCAGAG |
| 222 | JW13186_Guide1_3′ | GGUGCAGUUCCCCUUCAUUUGGGGAUGGUGUUGAGCUCCUAAAAUUCCCUUAGUCAGAG |
| 223 | JW13187_Guide1_3′ | UGCUGUAACAGCCCUGAUUUUGCAGGGUAAAUACAGUCUCCUAAAAUUCCCUUAGUCAGAG |
| 224 | JW13188_Guide1_3′ | CAAAAUGGAGACUUCAUCAGCUCCUAAAAUUCCCUUAGUCAGAG |
| 225 | JW13189_Guide1_3′ | ACCUCUCAAAAUGAGGCCUUCAACAACUCCUAAAAUUCCCUUAGUCAGAG |
| 226 | JW13190_Guide1_3′ | GUUGUAGAAGCUUAUCGUUUGGAUAGGUAUGACAACUCCUAAAAUUCCCUUAGUCAGAG |
| 227 | JW13191_Guide1_3′ | AAGACUGGUAUAUGAACCAGGGAGACAACAUCCUAAAAUUCCCUUAGUCAGAG |
| 228 | JW13192_Guide1_3′ | GUUGUCAUGCCGAUCCUAACGACAGGCUUCUACAACUCCUAAAAUUCCCUUAGUCAGAG |
| 229 | JW13193_Guide1_3′ | GUUGUAAAGACCUGCACUUUAGUAGGAAGAAACAACUCCUAAAAUUCCCUUAGUCAGAG |
| 230 | JW13194_Guide1_3′ | GUUGUAGAAGCCGUUCAUUCGGAGCGGUAUGACAACUCCUAAAAUUCCCUUAGUCAGAG |
| 231 | JW13195_Guide1_3′ | GUACUACACCCAUUUUGCAUGGGUCUAAAACUCCUAAAAUUCCCUUAGUCAGAG |
| 232 | JW13196_Guide1_3′ | GUUGUCUUUUCCUAACAAAAUAUAGGUUGCUACAACUCCUAAAAUUCCCUUAGUCAGAG |
| 233 | JW13197_Guide1_3′ | GUUGUAGAAGCCGCUCAUUCGGGGCGGUAUGACAACUCCUAAAAUUCCCUUAGUCAGAG |
| 234 | JW13150_Guide1_5′ | UCCUAAAAUUCCCUUAGUCAGAGGUCAUAGACCCCUUCAAUUUAUGAGGGGUUUUGUUC |
| 235 | JW13153_Guide1_5′ | UCCUAAAAUUCCCUUAGUCAGAGGUUGUGGAAGCCCUCAUUUUGAGAGGGUAUUACAAC |
| 236 | JW13154_Guide1_5′ | UCCUAAAAUUCCCUUAGUCAGAGGUUGUGCUUACCCUUAAAACUAAGGGCAGCUACAAC |
| 237 | JW13155_Guide1_5′ | UCCUAAAAUUCCCUUAGUCAGAGGUUGUAAGUGACCUCAUUUUGGAAGGUAAAAACAAC |
| 238 | JW13156_Guide1_5′ | UCCUAAAAUUCCCUUAGUCAGAGGUUGUGUAUAUCUCCCAAAUCAGAGACAGCUACAAC |
| 239 | JW13157_Guide1_5′ | UCCUAAAAUUCCCUUAGUCAGAGGUUGUUUAUACCUUACAAAAUAAAGGCUCUCACAAC |
| 240 | JW13158_Guide1_5′ | UCCUAAAAUUCCCUUAGUCAGAGCCUUUGGAGUACUGUUAUUUAACAGGGUUCUAAAAC |
| 241 | JW13159_Guide1_5′ | UCCUAAAAUUCCCUUAGUCAGAGGUUGUCGAAAGCCUUAUUUUGAAGGGCUAUUACAAC |
| 242 | JW13160_Guide1_5′ | UCCUAAAAUUCCCUUAGUCAGAGAUGACUAUACCAGCAAUGGCUGGAUUAAAAC |
| 243 | JW13161_Guide1_5′ | UCCUAAAAUUCCCUUAGUCAGAGGCUGUAGAGGCCCCCGUUUUAGGGGGUAGGUACAGC |
| 244 | JW13162_Guide1_5′ | UCCUAAAAUUCCCUUAGUCAGAGGCUGUUACUGCCCCACAAAUCGAGGCCUAUCACAGC |
| 245 | JW13163_Guide1_5′ | UCCUAAAAUUCCCUUAGUCAGAGGCUGUAGAGGCCCCCGAUUU |
| 246 | JW13164_Guide1_5′ | UCCUAAAAUUCCCUUAGUCAGAGGUUGUGCCUACCUCUCAAAUAUAGGGCUUUCACAA |
| 247 | JW13165_Guide1_5′ | UCCUAAAAUUCCCUUAGUCAGAGGUUGUAAAUACCCUACAAAU |
| 248 | JW13166_Guide1_5′ | UCCUAAAAUUCCCUUAGUCAGAGGAACUAUAGUAGUGUAAAUUUACACUACUCUAAAAC |
| 249 | JW13167_Guide1_5′ | UCCUAAAAUUCCCUUAGUCAGAGGCUGUUAUAUCCUUACCUUUGUAAGGGAAGUACAGC |
| 250 | JW13168_Guide1_5′ | UCCUAAAAUUCCCUUAGUCAGAGGUUGUAGUUCCCUUUUAUUUAGGGAUGGUGUUCAGC |
| 251 | JW13169_Guide1_5′ | UCCUAAAAUUCCCUUAGUCAGAGGUUGUAAAAUCUCUCGUUUUGGAGAGGAUUAACAAC |
| 252 | JW13170_Guide1_5′ | UCCUAAAAUUCCCUUAGUCAGAGGUUGUAGAAGCCUGUCGUUUGGAUGGGUAU |
| 253 | JW13171_Guide1_5′ | UCCUAAAAUUCCCUUAGUCAGAGGUUGUCAUACCUACCCAAACGAUAGGCUUCUACAACU |
| 254 | JW13172_Guide1_5′ | UCCUAAAAUUCCCUUAGUCAGAGCUUAUACAACACCCAUUUUCACAGUGGGUCUAUAAC |
| 255 | JW13173_Guide1_5′ | UCCUAAAAUUCCCUUAGUCAGAGGUUGUCAUACCGCUCCGAAUGAACGGCUUCUACAAC |
| 256 | JW13174_Guide1_5′ | UCCUAAAAUUCCCUUAGUCAGAGGUUGUCAUACCGCCCCGAAUGAGCGGCUUCUACAAU |
| 257 | JW13175_Guide1_5′ | UCCUAAAAUUCCCUUAGUCAGAGGCUGUCAUUACCCUCCAAAACGAGGGCUUCUACAGC |
| 258 | JW13176_Guide1_5′ | UCCUAAAAUUCCCUUAGUCAGAGGUUUCAGAACCCCGUAAUUCUACGGGGUUGUAGUUG |
| 259 | JW13177_Guide1_5′ | UCCUAAAAUUCCCUUAGUCAGAGGUUUUAGACCCCUGUGAUUCAGGGGUGUAGUGAAACU |
| 260 | JW13178_Guide1_5′ | UCCUAAAAUUCCCUUAGUCAGAGGUUUUAAGACUGUGCAAAUUCGCAUAGCUUUCAAUC |
| 261 | JW13179_Guide1_5′ | UCCUAAAAUUCCCUUAGUCAGAGGUUGUAGAAGCCUGUCGUUUGGAUGGGUAUAACAAC |
| 262 | JW13180_Guide1_5′ | UCCUAAAAUUCCCUUAGUCAGAGGUUGUCAUACCCACUCAAAUUGUGGGCUUCUACAAC |
| 263 | JW13181_Guide1_5′ | UCCUAAAAUUCCCUUAGUCAGAGGUUUCCAUACCCACUCAAAUGCGGGCUUCUGAAAC |
| 264 | JW13182_Guide1_5′ | UCCUAAAAUUCCCUUAGUCAGAGGUUGUGUGUGCCUUUCAAAUUGAAGGCGUUCCCAAC |
| 265 | JW13183_Guide1_5′ | UCCUAAAAUUCCCUUAGUCAGAGGCUGUACUUACCAUACAAAUUUAUGGCUUUUACAGC |
| 266 | JW13184_Guide1_5′ | UCCUAAAAUUCCCUUAGUCAGAGGUGCGAGACCAUCCCCAAAUGAAGGGGAACUACACC |
| 267 | JW13185_Guide1_5′ | UCCUAAAAUUCCCUUAGUCAGAGGGUGUAGUUCCCAUUUAGUUGGGGAUGUUCUCCCGC |
| 268 | JW13186_Guide1_5′ | UCCUAAAAUUCCCUUAGUCAGAGGGUGCAGUUCCCCUUCAUUUGGGGAUGGUGUUGAGC |
| 269 | JW13187_Guide1_5′ | UCCUAAAAUUCCCUUAGUCAGAGUGCUGUAACAGCCCUGAUUUUGCAGGGUAAAUACAGUC |
| 270 | JW13188_Guide1_5′ | UCCUAAAAUUCCCUUAGUCAGAGCAAAAUGGAGACUUCAUCAGC |
| 271 | JW13189_Guide1_5′ | UCCUAAAAUUCCCUUAGUCAGAGACCUCUCAAAAUGAGGCCUUCAACAAC |
| 272 | JW13190_Guide1_5′ | UCCUAAAAUUCCCUUAGUCAGAGGUUGUAGAAGCUUAUCGUUUGGAUAGGUAUGACAAC |
| 273 | JW13191_Guide1_5′ | UCCUAAAAUUCCCUUAGUCAGAGAAGACUGGUAUAUGAACCAGGGAGACAACA |
| 274 | JW13192_Guide1_5′ | UCCUAAAAUUCCCUUAGUCAGAGGUUGUCAUGCCGAUCCUAACGACAGGCUUCUACAAC |
| 275 | JW13193_Guide1_5′ | UCCUAAAAUUCCCUUAGUCAGAGGUUGUAAAGACCUGCACUUUAGUAGGAAGAAACAAC |
| 276 | JW13194_Guide1_5′ | UCCUAAAAUUCCCUUAGUCAGAGGUUGUAGAAGCCGUUCAUUCGGAGCGGUAUGACAAC |
| 277 | JW13195_Guide1_5′ | UCCUAAAAUUCCCUUAGUCAGAGGUACUACACCCAUUUUGCAUGGGUCUAAAAC |
| 278 | JW13196_Guide1_5′ | UCCUAAAAUUCCCUUAGUCAGAGGUUGUCUUUUCCUAACAAAAUAUAGGUUGCUACAAC |
| 279 | JW13197_Guide1_5′ | UCCUAAAAUUCCCUUAGUCAGAGGUUGUAGAAGCCGCUCAUUCGGGGCGGUAUGACAAC |

TABLE C
Nucleic Acid Sequences
Table 3-Nucleic Acid Sequences

| SEQ | | |
|---|---|---|
| ID | | |
| NO: | Name | Sequence |
| 280 | JW13101 | ATGAATCAGTACATACACGCTGATAAAAAAGAAAATAAAAAACGCCCCAACAAAAGCAGCATTATTCGTATCA |
| | | TGGTATCGGATTTTGACGATGAATACATTCAGGAAATCAAGGTGCTGTACACCAAACAAGGTGGCGTCGATACG |
| | | TTCAAAATAAACAAAATGAGCTATGACAGCGCCAGTCAGAAAATTATTTTTGAAGAAGTAGCAACACAAAACA |
| | | TGTTATCGGTGGAAGATTCAAATCTGAATTTTAAACGTCCAATGGTTGAGTGCAAAAATGGCGATATCTACGTC |
| | | GTTAAACCGTCGGGGAAAGTAAACGCAAAAGGACAGGCAATTGAACCTCTGAGATCTTATAAGATACATGGGA |
| | | AATACCTGGATCTGACAGAGGCAATTGGGAAAGACAACGCTGAATTGAGTGGCAAAAAGCAGATCTATCTTCAT |
| | | GTTGAGGATCTTTTAGGTATGCACACCACTGCCGATTCCATTGATCGTCGTAGACTCGAATCAGAGACCCAGAG |
| | | AACCCTGTTATCAAAAGAAGTGATGGAAAACTATGCTCTGATTATGGGCCACGAGATAAAGCTCAATGAATCCG |
| | | ATGAAACCTATAAACTGGCCTGTAGTAAAGAAATCTACAAGGCCAATCGGTTCTTAGACTATCGCTCACGCCTG |
| | | CTCTATTACTATAGCTTTATTAATCATTTTCTGGCGGGACTGTCAAAAGGGGCGACTTATGAGTACGCGGGAAAA |
| | | TCTTTGGTAACGGAGATTCCGGACGGTGAGGTGTGGCAATTATGTGAATTAGAGGTGACGGCGTACCATGGGAT |
| | | TACATTAACAAAAAAACAAATGAGCTTGTTAACGAAAATTGTCTCGAAATAGACGCGATCTATGCCCAGATGG |
| | | CAAAAAACATGAGAAAAACGTTAAACGAGTTCGTCGAAAATTATAACGCGATGATAGAAAAGCAAAACGAGCT |
| | | TAATAAAAACAAACCGCAGATGAAAAAAGCGATTGTGCAGGAACCGATTAAGTTCGAAAACTTTACAGAAGAG |
| | | AAAATCAATCAGGACTTGCACAATATTGTGTATATCCTGTCCGATCTGCGTCACAAACTGATGCACTTTGAGTAC |
| | | CGTTATTTTGATCTTCTGATGACGGGTAAAAAAATGGGCGAAAAAAGTGAAGTGGAAGTAAGTGTCCCTGATCG |
| | | GAATAGTGAGTTACCGGCTTCAAAGAATCAACCATCTAAGGATAAGTTCCGGAAAGGAAAGAAACTTTCAGAG |
| | | CTCTTAAACCTTAATATTCTGAAAGAACTCGACACATTTGTAAAAGTCAAAGAATCGTATCAGACCACTTATCTT |
| | | GAGACAAATGACAAAATAGAAATCCTGGGTAAGTTAAAAACTGCCAAATCTATCTATCAGATCTATCATCAAAT |
| | | TTGTCAGCGCAAAAACGGTTTCAACAAATTCATCAATTCCTTCTTTATTGTGGATGGTGAAGAAAATACAGAGGT |
| | | TAAAGACTGCATAAACGAGGTTTTTCGCAAGGAAATTCACTATTTCGAAACGGAAATAGCCAAATCCAATAAAG |
| | | ACACACTGGATGACAAGTATAAGAATAAAAATCAATCCCAGAAAACTCGTGACCGTATGGAAGATCAGATAGA |
| | | GGAGTGCAAACGTTATCAGGACGATGAATCCAATATAGATACTTGGGTTGCTTACCATAAGGATATTCATTATA |
| | | GCAAGCGGTACAAGAAACTGTACAGTAAACATAAAACGCTTGTAGGCAATCTGAACAGCGCTGTTTCCAAGGGT |
| | | TTAGACGGTCAGGTTATTAAGAAAATCAATGACGACATTGCAATTATAAAGCGCGAAATGAATGAAATTACCAA |
| | | AGCCAATTCAAAATCTCGGTTGCGCTATAAGATGCAAATGGCCTATGGCTTTCTCTTTGTTGAGTATGGTCTCAA |
| | | AATTCCGAAATTTTTAAACGATTTTGATCTGAGCCATGAGCGCACAGCGTCAAAAATAAAAGGATATACCTCGC |
| | | CAGAAAAAGTGACCCAATATCTGACCAATGGTGAAGGAAATAAAGACAATTTTAATTTGGATGAACTGACGAA |
| | | GAACTTTAAGAAGCAGATGAAGTTCGAGTTCCTGAAACTGAACGAAGATAATAACTTGATCAAACTCTACATTT |
| | | TGATCTATCAGCTTTTGCCACGAGAATTAAAAGGTGACTTTCTGGGTTTTGTTAAAAGCAATTATTACGATCTTA |
| | | AGCACGTGGATTTTCAAACACGAGAAACCGAAGCAAAAGATCAATTTTTCCACAACATGCGCCTGTTTGAAAAA |
| | | AACGTGAAAGCATTTGACTTGATCCAATATTCAATAGGGGATGAAATGTCTCAATTGGGCAATGAGACCTTTAA |
| | | TTTTTCACAGGCGCTGTCAAAAATAGTATCCGACGATACCATCCTTAATAGTGCTACCGAGATTCCCAACATCAA |
| | | TTGTCTGGTTTATGGGAGCTTATTAAAATACTACGAGAATGCTTTTCGTCTGTCTAGTGAAATCGAAATTCGCGC |
| | | CTTAATAAAAATTGCACGAAAAGAGAAAGAGGTGGTTGATCATTCCAATAGCTACTCGATTAACAAGGCACATA |
| | | GCAAAATTGAAAAAGAATTAAAAGAGCGTGATAGAGATAAGGAAGTCTCATTCTTTTGCATCCTCGAGTACGCC |
| | | CATAAAAATTCTCTTAATCAAAAAGGCGACTATACTGATTCTGAGATCTATGATAAAGCTTCCAACTTACGAAAT |
| | | AAGATTGCCCATTGCGATTATAGAAACCTGTTCATTACAAATATTATTTGTGAAGCGGAAAACATTAACGTGAA |
| | | GGTAAAATATCTGATTGACGTAAGCAACACAATCGGCCTTAACACTGTCGATCTTGGCAATGATATGGTGAACG |
| | | ATTACTTGATGTCTTATGATAAACAGATGACGTATCTCGTTAAAACCTCTGAAGAGCTTCTGAAAGAAAGCCGTT |
| | | TGGATAAAAGCAAAGAAAAAAAAGAACGCCGCGACAATCTTAAAAATGAAACGAGAAGTCATTCAGAAATCTA |
| | | CATGGAATATGAATGGATTAGTGATTACCTCGTGAAGTTGAAAGACAATCACGAGATCTTATTGAAAAAAAATA |
| | | AGACTGAGGATCTGAAACTGAATCGAGCGTATGAACTGATAAAAAACTATAATGTCGCTCTGAATAATAAGTCG |
| | | AAGATTAAGATAAGTGAGCTGACGGCAGACCATATCAATAACAATTGGGTGAACATCTGGGGAGTACTGAACC |
| | | ATGAACTGCAGGAAATTAACGGCCTTTATTTGTATAAGGTCACTCCTAAAATCAAAAAAGGGATCTACCTTGTTT |
| | | TAACCGATAAAAAGAATTACTGCCTGAATATTAACTTCTATACGCTTAAGAAAGTCCCTAGTAAAGATGATTCT |
| | | GAAATGACTGAAGAGCGTTATCTGCGTGATATTAGTGAAAAGTATTATTTTACCGTTGGTGAAGATAGTATGAA |
| | | AGCATTAAAAGATCGGTTGCAGACTATCAATGATAAGTGCATTGTTAACTATGTCAACAAAATTGACTGTAAGA |
| | | ATGAAAATCTCAAAGGAAAGTCGGAATTCAAAGTAATCGACGTTTATAATTCTTGGGATAAAAAATATCTGAGC |
| | | ATTTCAAATGAAGAGTCGCAGCATTGGTCTGTTGAGACAAAACGGGATAAGAAATACAAAGAAAACCTGATAT |
| | | TAAACCTGATGGAGAAGTCCTCTTTTAAGCTTAATCTGCATCAGCTGTAA |
| 281 | JW13102 | ATGGCGCATAATCCGACATATTTGTATGCCGCGTATTTGAATATGGCACGTCATAATGCCTATCTTACCCTGTCC |
| | | TACATCAGCGTTGCTATTGGCGGAAAAGAAGGCGCAGAGGATAAACTGCTCAATATGGAAGCTCTGAATATTCT |
| | | CAAAAAGCCAAAAGCAGAACCTGAAAAATTAAAGAAAACAGAAGAACTGTTGACCAAACATTTCCGTTTCCTT |
| | | CTCCCGATGACGGATAATGATAAAAGTGCCGGTTATCATGAAATTTTAACCACTGCATTCAAATTGCTCAATTCT |
| | | CTGCGGAACGAATATACGCACTATCAGCATGAAGCAAGCGATTTTGATGAACAGTTCATGAAAAATATGAAAA |
| | | ACTGCTTTGATGGGGCAAAACGGACGGTTAAGGAACGTTTCAATCTTGAAGACAACGATTTGAAGCACCTGGAC |
| | | CGGTTTAGACAAAAAGCGAATGATGGTAAAAAAGGCAAACGTGAATATGAGGAAAATCCAACTTTTCACTATA |
| | | AGTTTGATGATGAGAATGGCAGACTGACCGAAAAAGGCTTGGCGTTTTTTATTTGTCTGTTTCTCGAAAAGAAGT |
| | | ACGCTACACTCTTCCTTACTCAACTGTCCGGTTTCAAAAAAGGCGATACCAAAGGCACCAAAGCGACCAAAGAA |
| | | GTTTACTCGATCTACCGGATACAGCTGCCCAAATTAAGAATCGACAGCAAAAAACCGGAAATGGCTCTTGGTAT |
| | | TGATATGTTGAACGAGCTGAAAAAATGTCCCGCCGAGCTGTACGATGTATTAAGTACGGAAGACCAGAAACGCT |
| | | TTCGCATTATTCCCGATGAAGAACCGGACACGATGGACGATACGGAAGACGGCGAAATTCTGCTGAAACGACA |
| | | CAAAAACCGTTTTCCATACATGGCATTGAGATACATTGACCAAAACGAGCTTTTTCCAAATATTCGTTTTCAGGT |
| | | AGCTCTGGGAACTTATCGCCATCATTTTTACAACAAACTGGGGGTTGATAATATCCAGCGGGTGCGCATCCTGCA |
| | | AAAACAACTGAAAGGTTTCGGACGTCTTGACGAGATAGAGGAAAAGCGAAAGACCGTATGGGCAGACAAAATT |
| | | CGCCCTTTTGAAAAAATTGAGAAAGACTCTGCCGAACAAGAGCCTTATATCACAGACAGTTACGCCAAATATAT |
| | | TATTTCAAACAACAAAATCGGTCTGAAATGGTTCGACAGACAAAATGATTATTATCTTCCCGAGTTAAACGAAA |
| | | AAGGGGCGAAAAATGAAGCGCCCACATGTTGGTTATCCATCTATGAACTTCCGGCGTTACTGTTTCACAGCATC |
| | | CTTTGCAATCATAGTTCTGACACCCAGAATATTATACGTGATTATGTATCGAAGGGATACCGAAAACTGTTTAAA |
| | | GAGGTAGCATCCGGTGCGCTGCAGCCCGCCTCAAAAGAAAAAATCGTCAACCAACTTAAAACAGCCTATAATCT |
| | | TGATTTTACCCGCATACCGAAAGAATTGCAGGATTATCTCACCGGAAAGCCGGTTAAAATGGATGACCAGCTGA |
| | | ATAAACTGTGTTCGGAACGTATTGCTCAGATGGTCAGCGATTCACAGAAGCGACTTGAGCGCATAGATAACGAT |
| | | CTGAAAACTATCAAGGATAGAAAGAGCAACAAGGTCGGGAAGAAACGATTTGTAGAGATTAAAAGTGGTGTCT |
| | | TAGCCGATTTTCTGGCCGAGGATATGCTGGCATTTCAGTCCAGTTCGGACAACGGCAAAGATAAGTTGACGGGC |
| | | ATGAACTATCAGATATTACAAGCTGCACTTGCATATTTCGGTCAGTACAAAGACCAGATGACAGAAATCTTTGA |
| | | GAAGTGCAAGTTGATAAACTCACCAATCGAACATCCGTTTCTGAAGAATGTGATGGCAAAAAATGCTACCAACA |
| | | TTGTGGATTTCTATATCGTTTACTTAAAGGCGAAGGAAATATATCTGGATAAGTGTCTGCGGGAGAAACAATAT |
| | | AAGAACGATTATTTTTTACATGTTGACCGCCAAAAATGGACGGAGCGCAATACTGATTTTTACAAAGTGTTGGC |
| | | AAAGAGCTATGCGGAGAAATTGCCTGTTGAATTGCCGCGCGGTCTGTTTAACGATGCCATTAAAAAGCTGTTGA |
| | | CGAAAAAGCACGGTGATAATCCGGTGATTCGCGAAGCTCTGTCAAAAGAGCGGTGCAATACAACTTTCCTGATT |
| | | CAGACTTATTTCAAAGCTATCTGTGAAGACACCACACAGGAATATTATACATTCAACCGCAGTTATAAGATTTTC |
| | | AATTTGCTCAATGATAACTACGTCGGAAATAAACTTCAGCCTGCCTATTATGCTGTTAATGAATTTGAGAAAAGA |
| | | GTCAAAAACATAGATGCGGAAATAGAGAAATTCGACTCTCAGCAACACGATAAACTTCATCGTCTGTTACATCA |
| | | TTTCAAAGAAAATGAAAAAATACTCCGTTTATACAAAGTGCAGGATATCTTACTGTTTTTGATGGCAAAACGCA |
| | | TACTCATCACGGAGATGGGTGAGAATAATATTGGGAAATACCAGTTAAAACAGATTATGCCGGATAGCCCAACG |
| | | GATATTTTATCTGCTCAAACATCATTTTCAATCACTCTGACCAAAAACGGATGCCAGAAGATCATCAAACAGGA |
| | | GAAACTGAAACTGAAAAATTTTGGCGATTTCCAGCGTTTTGTGCGTGACCGTCGTATCGAGACTTTATTTCCATA |
| | | TATGAAAGCGGATAGTATTGACCGGCAGATACTGGAAGCCGAGCTGGATGCGTACGATGATAAGCGCGTGCAT |
| | | ATTTTTGAACTCATACATCAGTTTGAGGAGTCTGTGTTGACAAAGAATCCATCCATTTTATCAGGGAAAACCTAC |
| | | ATCAATTTCGCCGGTATTTTAAAAGCATCTCCTGAGATTCAGACAGAAAAACGTCAGCAAATGCAAATTATCCG |
| | | CAATGCCTTCTGCCACAATACTTATCCTCAAAAGATTGAAAAGAACATTGAAGACATTGAAAAGCCGGAAGGGA |
| | | AAACCGATGTTCCTTTTTCTTTATGTGAAAAGAAAGAAATACCGGAAATTGCTGAGTTTCTGACGGAATCGCTTG |
| | | AAGACTTGATCATTATAAACGAACACTAA |
| 282 | JW13103 | ATGGATACTCCTAATTTTTCTGAGCGTATCCCGGTTTCTTTACAAAGCCATCCTTATTATTTTGCGCACTATCTTA |
| | | ACATGGCCCGCCACAACGCCTATGTGATTCTGGAGTATGTGAATAGAGAGTTAATAAAGCCAGGTAAGAACCTG |
| | | GACGAAGACAACCTGATCCAGAGCACGGTGCTGAAAGACGGATATTTTGATCGTAAACCTGACGAGCTCTCACA |
| | | CCGGAATCGCCTGCTGGTCCAACATTTTCCTTTTTTACGTGAGGCAGAAAATGAAGGTGCACGCACGTGCAATCC |
| | | GGTTAGCTATAAACTGAAAACCGCTTTAGCAGCACTGAACCAGTGGCGTAATAATGCATCGCATTATCCGCTCA |
| | | ACCAGAATCACGAGAAAGACTTTGACCTGCAGCCGTTTTTTTCCTTTGCCATTGAGGCCTGCAAAAAAAGAATG |
| | | CGGGAAGTTTTTCAGCCAGATGACTTTTATCTTTTGGAGACCAACGAGAAACAGTTCTACACGTTACATAATGAA |
| | | AACGGTTTCACAGAAAAAGGGCTGTACTGCTTTATTTGTTTCTTCCTTGAGAAAAAGTATGCGTTTCAATTCCTC |
| | | GCCGGCATTAAGGGGTTTAAAAATACAACAGATAATAAGTTCCGCGCTACCTTAGAGACGTTTACCGAGCATTG |
| | | TTGTCGATTACCAAAACCCAAACTTGATTCTTCAGATATTAAACTTGACATGCTGGGCGAACTGTCTCGTTGTCC |
| | | CGCCCCTCTGTTTGATCTGCTGGATATCGAAGAGCGGAAGAAGTTCATCCGCGAACCCGAAGAAGTAAAACCGG |
| | | ACGAATCCGGTGATAGAGAAGAAGTGCAGCAGGTGTTAATGAAACGTTACGATGACCGCTTCCCGTATTTTGCG |
| | | TTACGATATTTCGAGGAAAAAAATCTGCTCAAAGGGATCTCATTCCATATTCACATAGGCAGATGGATAAAAAG |
| | | TGAACATACGAAAAAAATTATGGGCGCCGAACGTGATCGTCGTTTGCTGAAAGATATTCGTACATTTGGCGAAC |
| | | TGAAAGAATTTTCTCCTGAACACGCTCCGGATTATTGGCTGCGCGACGGCATTACTCCTGATGATGTGGATCAAT |
| | | TTTCAACGCCGCAGTATCGTATTGTTGGAAATCGAATTGGAATCAAACTTAATTACAACGGCCATAATAGATGG |
| | | TCGGTTCCGGATAAAGAGATTAACGTAAAACCTGATGCTATCATAAGTACGTACGAATTTCTGAACTTGTTTCTT |
| | | TACGAGCATCTTTATCAGAAAAAACTGACCGGCTTAAGTCCGGCCGAGTTCATTCAGGATTACCTTGATCGCTTT |
| | | AATAATTTCCTCTCTGAATTCAAAGCCGGTCACATACGTCCAGTTGGAGATTTTTCGTTAGAGAAACGTCGAGGG |
| | | CAAGGGGACGAACCTGATCTTACTGCGCGTCGCAAAAGTTTGCAGAAAGAATTAGATCGATTTGTCTTAAAAGG |
| | | GAAAGACCTTCCTGACAAGATCCGCGAATATTTGTTAGGCTATAAACAGAAGAGTGAGAAGAAACAGGCGAAG |
| | | TGGATACTCGGTGGTATGATTAAAGAAACCGTCTATTGGCGTAATAAAGCAGAACAGTCCCCGGAAAAAATGCG |
| | | AAGCGGAGATATGGCCCAACAGCTGGCTCGGGATATCATTTTTTTAACGCCGCCACACACAGTTAAGGAACACA |
| | | AGCAGAAATTGAACAGCTTGGAGTATGATGTACTGCAGTATGCACTGGCCTATTTCAGCTCAAATCGCGAGAAA |
| | | CTTTATTCTTTCTTTAAAGAACATCAACTCACTGTCAAAGGGGACCGCGCACATCCATTTCTTTATAAGATTCGG |
| | | CTGGACGAGTGTCAAGGTATATTAGACTTTTTCATCGTGTATATGCAGCAGAAAGAAAAATGGCTGGGGTGGCT |
| | | GGACCGCAACTTGAAGTCACCACGTTTGAACGAAGAGGAGTTCTTTAACACTTACTCATATTTTATTAAAACAG |
| | | ATACTAAGCGGGCAATTGAGATGGATTATGAAAGTTGCCCAAATTATCTTCCGCGAGGTATTTTTAATGAACCTA |
| | | TAGCAAAAGCTGTCCAAAAAGCTGGTGTGAAGATTAAAGACGAAGATAATGCGAGTTATGCGTTATCCGTATAT |
| | | AGCAACGGCAAGACTCAACCCTTCTACAATAAGGAACGGTACTATAATAAAGGTATATTTCGCATGGAAGAACT |
| | | GCCGGAAAAGCTGCAGCCCAAGGAATTGTTAGGTAAAATACAGTGGACAATCAAGAGTAGCGGTAAAGATACT |
| | | GAAGAATTTCGGTCGTTACAGAACCTGAAAAACCGTATTCTGAACACCGAGAAAGAAATTCGTTATGTTCAATC |
| | | CACAGATAGAGCGCTGTGGATTATGGTTGCTGATCTTTTTCCCGAAACCTTCGAGCTGAGACCAGATGATCTGGA |
| | | GTGTATAGGACATGATCTTTCTGATGATCTTTTAAGCCGCCCCTATCAGATGAAAGAGAAAGTCTACAATTACAC |
| | | AATTACAGACTATTTGCCGATAAAGCGGTACGGAGAATTTCGCCGTTTCTTAAAAGATAGACGTCTCGAGAATC |
| | | TCTTAACCTACTTTGAAGAAGGCGTACCGCTGCATCGGGAAGCTCTGGTAGCGGAGCTCGAGGCGTACGACCTG |
| | | CAGAGAAAAAATTTGTTGGAGATCATCTATCGGTTTGAGAAGTTAGTATTCGACCGCCATCGGCATGAACTGAC |
| | | TTTCTCAGGTGAGGGCGAAAATCAATACGTGAATCACTGGGATTACTTGGATTTTGTTGCACGTAAGTACGGACT |
| | | GTCGGCAGAAGTGAAAGAACTGAACTCCGAACGTTTTACGGAACTCCGCAACAAAATGCTGCATAATCAGATTC |
| | | CATACCAGTTATGGATCAAAGAAGCCATCGCTGCGAGAGAAGAAAATACCGTCTGCGGTAGAATTATGGGAAT |
| | | GATCGGCGAAATCTATGAACGCATGACCACCGAAATCGAAAAACAAATGCAAGTTTAA |
| 283 | JW13104 | ATGGAAAATCAGACACAAAAAGGAAAAGGTATCTATTACTACTATACCAAAAATGAAGACAAACACTATTTTG |
| | | GCTCCTTTCTGAACCTGGCGAATAATAATATCGAGCAAATCATTGAAGAGTTCCGGATTCGTCTTAGTCTGAAAG |
| | | ATGAAAAAAACATCAAGGAAATTATCAACAACTACTTCACTGATAAAAAGTCTTACACGGACTGGGAACGTGGT |
| | | ATAAACATATTGAAAGAGTATCTTCCGGTTATTGATTATCTCGACTTGGCTATTACCGATAAAGAGTTCGAGAAA |
| | | ATTGATCTTAAACAAAAAGAGACCGCAAAACGCAAATACTTTCGGACAAATTTCTCACTGCTGATCGATACTAT |
| | | CATTGATCTTAGAAATTTTTACACGCACTATTTTCACAAACCTATCTCAATCAATCCCGATGTAGCTAAGTTTTTA |
| | | GACAAAAACCTTTTAAACGTGTGTCTCGACATCAAAAAACAAAAAATGAAAACCGATAAAACCAAACAGGCTT |
| | | TGAAAGATGGTCTGGATAAAGAGCTCAAAAAGCTGATTGAATTAAAAAAGGCAGAGCTGAAAGAGAAGAAGAT |
| | | TAAGACGTGGAATATTACGGAAAACGTTGAAGGAGCTGTTTACAACGATGCGTTCAATCACATGGTTTATAAGA |
| | | ATAACGCGGGTGTTACAATACTGAAAGACTATCACAAAAGCATATTGCCCGATGACAAAATTGACAGTGAGTTA |
| | | AAACTGAACTTTTCAATCTCCGGACTGGTCTTTTTACTTTCTATGTTCCTGTCAAAGAAAGAAATTGAGCAGTTC |
| | | AAATCTAATCTCGAAGGCTTTAAGGGAAAGGTCATAGGCGAAAATGGGGAGTATGAAATTAGCAAATTCAACA |
| | | ATAGTCTTAAGTATATGGCGACACATTGGATTTTTTCATATCTGACTTTCAAAGGGCTCAAACAGCGGGTAAAAA |
| | | ACACGTTTGACAAAGAAACCTTGTTAATGCAGATGATTGATGAACTGAACAAAGTCCCTCACGAAGTTTATCAA |
| | | ACGCTGAGTAAAGAACAGCAAAATGAGTTTTTAGAAGACATTAACGAATATGTGCAGGATAATGAGGAAAACA |
| | | AAAAGTCAATGGAGAATTCGATTGTCGTGCATCCGGTTATCAGAAAACGCTATGATGATAAGTTCAATTATTTTG |
| | | CGATTCGTTTCCTGGACGAGTTTGCAAATTTTCCTACTTTAAAGTTTTTTGTTACAGCAGGTAATTTTGTACACGA |
| | | TAAAAGAGAAAAACAGATTCAGGGAAGCATGCTTACTTCTGATAGAATGATAAAGGAAAAAATCAATGTGTTT |
| | | GGCAAACTGACTGAAATTGCAAAGTACAAAAGCGATTATTTTAGCAATGAAAATACACTTGAAACTTCTGAATG |
| | | GGAGTTATTTCCTAACCCGAGCTATCTCCTCATACAAAACAATATTCCGGTGCATATTGATCTTATTCATAATAC |
| | | CGAAGAGGCAAAACAGTGCCAGATTGCGATTGACCGAATAAAATGTACGACGAATCCGGCTAAGAAGCGCAAT |
| | | ACTCGCAAATCCAAAGAGGAGATAATAAAGATTATCTACCAGAAGAACAAGAATATCAAATATGGCGACCCGA |
| | | CGGCACTTCTGAGTAGTAATGAGTTGCCAGCATTAATCTACGAGTTACTGGTCAATAAAAAATCTGGTAAAGAG |
| | | CTGGAGAACATTATAGTAGAAAAAATCGTCAATCAATATAAGACGATTGCCGGGTTTGAAAAAGGGCAAAATC |
| | | TGTCAAATAGTTTAATCACAAAAAAACTGAAAAAGAGCGAACCGAATGAGGATAAAATTAACGCCGAAAAAAT |
| | | TATTCTGGCCATTAACCGAGAGCTGGAAATCACCGAAAACAAACTCAATATCATAAAAAATAATCGGGCAGAAT |
| | | TTCGCACAGGTGCGAAAAGAAAACATATCTTTTATTCTAAAGAGTTAGGGCAGGAAGCGACTTGGATAGCATAC |
| | | GATCTTAAACGTTTCATGCCCGAGGCTTCCCGTAAAGAATGGAAAGGTTTTCATCATAGCGAACTCCAGAAATT |
| | | CTTAGCTTTCTATGATCGCAATAAAAATGATGCGAAAGCTTTGCTGAACATGTTCTGGAATTTCGATAACGACCA |
| | | ATTAATTGGCAATGATCTTAATTCAGCCTTCCGTGAGTTCCACTTCGATAAGTTTTATGAAAAGTATCTGATTAA |
| | | GCGGGACGAAATACTTGAGGGCTTCAAGAGTTTCATCTCGAATTTTAAAGATGAACCTAAGCTGCTTAAAAAGG |
| | | GCATTAAAGACATCTACCGTGTGTTTGATAAACGATACTACATAATTAAAAGTACAAACGCCCAAAAAGAGCAA |
| | | TTGCTGTCCAAACCAATCTGCCTGCCACGTGGTATATTTGATAACAAACCAACATATATCGAAGGTGTTAAAGTT |
| | | GAGAGCAATTCGGCTCTGTTTGCCGATTGGTATCAATATACATATTCCGATAAACATGAATTTCAGTCCTTTTAC |
| | | GACATGCCCCGGGATTATAAGGAACAGTTTGAAAAGTTTGAACTGAATAACATCAAAAGCATTCAGAATAAGA |
| | | AGAATCTGAATAAAAGCGATAAGTTTATCTATTTCCGATACAAACAGGATTTGAAGATTAAACAGATAAAGTCG |
| | | CAGGACTTGTTCATCAAACTCATGGTGGACGAATTGTTTAACGTCGTGTTCAAAAATAACATAGAACTTAACCTG |
| | | AAGAAGCTGTATCAAACCTCTGACGAACGCTTTAAAAACCAGTTAATAGCCGATGTACAAAAAAATCGTGAAA |
| | | AAGGCGATACATCGGATAATAAAATGAACGAAAATTTTATCTGGAACATGACCATTCCGCTTTCATTATGTAAT |
| | | GGTCAGATTGAAGAACCGAAAGTAAAACTTAAAGACATTGGTAAGTTTCGTAAACTGGAAACCGACGATAAGG |
| | | TCATTCAGTTGCTTGAGTATGATAAAAGTAAAGTGTGGAAAAAACTCGAGATCGAAGACGAATTAGAAAACAT |
| | | GCCAAATTCATACGAACGTATACGCCGTGAAAAATTGCTGAAAGGAATCCAGGAATTTGAACATTTTCTGCTCG |
| | | AAAAAGAAAAATTTGATGGCATAAACCATCCTAAGCATTTTGAACAGGACCTGAATCCAAACTTCAAAACCTAT |
| | | GTGATTAATGGAGTGTTACGCAAAAACTCGAAGTTAAACTATACCGAAATTGATAAACTGCTCGATCTGGAACA |
| | | TATTTCAATAAAGGATATTGAAACCTCTGCCAAAGAGATTCATTTGGCGTATTTTCTTATTCATGTACGCAACAA |
| | | ATTTGGGCATAACCAGCTGCCGAAACTGGAAGCCTTTGAGTTGATGAAGAAATATTACAAAAAAAATAATGAG |
| | | GAGACTTATGCCGAGTACTTTCACAAAGTTAGTAGCCAGATCGTTAATGAATTCAAAAATTCCCTGGAAAAGCA |
| | | TTCTTAA |
| 284 | JW13105 | ATGAAAATTAAAAACGAAAATACCGATAAGAAAACTGAACTCTATTCGATCGATAAGTACAAAGGCCGCGATA |
| | | AGTGGTGTTTTGCTATCGTGCTTAATAAAGCACAAACAAATCTTACGGAAAATCCTGATCTGTTCGAGCAAACC |
| | | ATAACGAAATATGACCGGATCCGGAAAGAAGGTTGGTTTGACGAGGAAACAAAGAAATTAATCTATATCCAAG |
| | | AGAATGAGCACAAAATCAAAGGAGAAATAAAAACTCTGGCCCGTGAGGTATTAAAAAATCTGCGCAATTACTT |
| | | TTCACATCACTTTTACAAACAGGACTGCCTGATTTTTCCAAAAGATAACATTGTGCGTATTATTATGGGGCGCGC |
| | | ATACGAACGAAGTGAATATGAAATCAAGAAAAATCTGAAGGAAGATATTTCGATCGAGTTACCCGCTTTATTCG |
| | | AGCCAGAAGGCAAAATTACCACGGCGGGTATAGTATTTTTTACTTCATTTTTTGTCGAACGCCGATTTTTACACC |
| | | GTTTGATGGGTAGTGTTCAGGGGTTTACCAAAACCGAAGGAGAGTACAAAATAACACGTGATGTTTTTGCTAAG |
| | | TATTGTCTGCGTGACAGCTATTCAGTTAAAGCGCAGGATAACGACGCAGTTATGTTTCGCGATATTATTGGTTAT |
| | | TTGTCACGAGTTCCAACGGAGTCCTTCCAACATATTAAAAATCCGAAGAAACAGAATGAGAGTCAGTTAAGCGA |
| | | AAGAAAGACAGACAAGTTTATCTCTTTCGCCCTGAAATATCTGAAAGACTATGGCTTTGAGGACTTGAAAGGTC |
| | | ACTACACGGCTTTCTTTGCGCGTAGTGAGATTAAGAAGGAAAAAGAGGATATCGAAATTAAAGATGACAAAAA |
| | | ACACAAGCCTCACCGTATGAAATCCAAAATTGAGATTCATTTCGATAAAACAAAAGAAGATCGCTTCTACATTG |
| | | AGCGCAATAACGTCATTTTGAAGATTCAAAGAAAAGGCGGTCGTGCCAATATATTAAGAATGGGTATCTATGAA |
| | | CTTAAGTACCTGGTGCTCCTGTGTTTGTCCGGGAAAGCACGCGAGGCGATCAATCGCATCGACGATTATCTGAA |
| | | CGATCTTCGGAACAAAATTCCTCATATTGAAAATATGAACAAAGAGGGTATTGGCGAACAAATACGTTCCCTGC |
| | | CGGGATTTGTAAGAAGCCAACTGGGCTTTGTTCAGATTGATGATGAGAAAAAAAAGGAAAACCGTCTGGACTAT |
| | | GTTGAAAAAAAATGGGAAAAAAAGAGAGCCGAATCAAAGGAGCTGAAGTTGAATCGAAAAGGGCGGGACATA |
| | | CTCCGGTATATTAATGAACGTTGTAAAAAGCCGCTGACAATCGATCGTTATAATCGCATTCTGGAATTGCTGGTC |
| | | GAGAAAAATATAGAGGGATTTTATCATGAACTCGAAGAACTGAGAAAAACCGGTCGTATAGAGAAAAATATTA |
| | | CTCAGGCACTTGTCGGAGAAAAAAACATTAATGCATTACATATTAAAATTTGCAAACTGGTGCAGGATGAACTG |
| | | AAAAGCTTAGAAAAAGAAGACCTCAAGAAATATATCGGCTTGACTCCCAAAGAAGAAAAAGTCGTGTCGTTTG |
| | | AAGAAAAACTCGGTCGCATCCTGGATAAGCCGGTTATCTATAAGGGATTTCTTCGCTACCAGTTCTTCAAAAATG |
| | | ACAAAAAGTCCTTCGCGCGCCTGGTTGAGGAGATAATCAAAGAAAAAACCGGCGGTTTAGATGTACCCATTGAA |
| | | ACAGAGTATTATAGCATTTCAACCCTTGGCAGATTTGATAAAGCTAACAAAACGTTATATGAAACGTTAGCGAT |
| | | GGACCGACTTTGCATGATGATGGCCCGTCGCTATTTTCTGAGTCTGAATAAAATACTCGCAAAACGTGCCCAGA |
| | | ATATCGAATGGAAGAAAGAGAGTGGTAAAGAATTCATTGTGTTCAAGTTTAATATGCCTCAGGATACAGGAAAA |
| | | AGTATTTCGATCCGGTTCTCTCCAAAAGATTATACTAAACTTTATGTGAAGAATGATTCTGAATTCCTGGCCCGC |
| | | CTGTGCCAGTATTTTTTTCCGAATGAAAAAGCGATTGATTACCATAAGTTATACTCTCATGGCATAAACAAATAT |
| | | ACCAACTTCCAGAAGGAAGGGATCGAGGCTATACTTGAGCTCGAAGAAAAAATAATCAAAAAACGGAAAATCA |
| | | ACTCTCCGGAGAACTACTTATCATTTGAAGAAATACTGAACCAAAGCATCTACAACGATGAAGAAAAGAATACT |
| | | TTGATTCAGATTCGGCATTCTCTTTTGCATTATCAGATACTTTTTAGCAAAAACGATCTGACCAAATTCTACAAC |
| | | GTAATGAAGCGTGAGGGGATTGAAAAAATTTGGTCTTTAGTGATTTGA |
| 285 | JW13106 | ATGATCAAAAAACCCGCCAATCGGTTTGCCTTACCTAAAGTGATTATCTCTGAAGTGGATAGTGAGAACATTCT |
| | | GGAATTCAAAGTGAAATATGAAAAACTGGCACGGCTTGATCGCTTTGAAGTTGAAGCGATGCATTATGATGAAA |
| | | AGAATGAGATTATTTTTGATGAGGTCAAAGTGAACGATGGACTTATCGAAATTGTTTACAAAGAGAACAATAAA |
| | | AAGCTCGTTGTGGAAATTGAAGGCAAAGCATACGAAATTTCAGGCCAAAAAATAGGCGGAAAACAGCGTCTGC |
| | | TTGAGAACCGGGTAAGTAAAACCAAAGTGCAGCTGGAGCTTATGAATCATATAGAAGATGGAGAAGGTAACAA |
| | | ACGCGTCAGCCGTACCGAGCGCGAATTAATTGTTCTGGGTGACATTAAACTCTATTCTCAAATCGTCGGGCGAG |
| | | AAGTTAAAACGACTAAAGAAATCTATCTGATCAAACGCTTTCTGGAGTACCGCTCAGATTTGTTATTTTACTATG |
| | | GGTTCGTAGACAATTTTTTTAAAGTCGTTGGGAATGAAAAGGAACTGTGGAAGATTGACTTCAATACTTCTCATT |
| | | CCGAACAGCTTATCGAATACATTAAGTATACCTTCAATGACAATTTGAAAAACGATCGTCAGTATCTGAATGAA |
| | | TATGTTACGAATGATGTGCAGATACAAAAGGACTTTAATAAAGTTAAGAACGTTTTTTCAGAGCTGCGTCATGC |
| | | CTTGCTGCACTTTAACTATGATTTTTTCCAGAAACTTTTCAACGGGCAGGATGTGGGCTTTGATTTGGACATCGA |
| | | GTTTTTAAACCTGTTCATTGATAATATAGACAAGCTGAATATAGAGACGAAAAAAGAGTTTATCGGAGATGAAA |
| | | AAATCCGACTTTTTGGTGAAAACCTGAGCCTTGCAAAAGTGTATGAACTCTATTCCCATATTTGCGTGAACAGAG |
| | | TTGGATTCAACAAGTTTATCAATAGTATGCTCATAAAAGACGGTATTGAAAATGAAGCCCTGAAAGACAAGTTT |
| | | AGTACCAAGTTTACTGGAAATGCCTATTATATCGATATTCATAATTCCCGTGAGTATAAAGACCTCTATAAGAAA |
| | | CACAAACGTCTGGTGATTAACGTTTCAACACTCAAAGATGGGAGAGCCATTCGTCAAGAGAATAAAAAAATAG |
| | | CTGAACTGAAAAAACAGATGACCACCATGACAACTACGAATTCTTTGGCTCGATTAGAATGTAAACTGCGGCTG |
| | | GCATTCGGCTTTCTCTACAATGAATACAATGACTACAAAGCTTTCAAAAATTCATTCGACTCGAACCTGAAAAAC |
| | | AATCAGTTTGACGTTAGCAATGTTGATACATCGAAAGCGTATTTCCTTTCTACTTATGAGAACCGTAAACCACGC |
| | | ACAAGAGAACGTATTGACAAAGTTTTAAAAGAAATTGAATCTCTGGAGCTGAAAGGGCTTATCGCGAATGATCC |
| | | CCTGTTAAAATTCATCTTGCTGATGTTCGTCTTTATGCCGCAGGCGTTAAAGGGTGAATTTCTTGGTTTTGTAAAA |
| | | AAGTACTATCATGATGTACACAGCATTGAGAAAGATACGAAAGATAAAGAAGAGGACTTAATAGAATTCATGC |
| | | CGTTGTCGTTAAAACTGAAGATTTTAGGCCGGAACATACGAACCTTAACACTGTTTAAGTATGCGCTCGGCTCAC |
| | | CGATAAACTATAATAGCACCGACGAAGTATTTTATGTCGAAGGAAATCGTTATGGTAAAGTTTATAAACAGTTG |
| | | GGTATTTCTCACAATCAAGAGGAATTTGATAAAACACTGACGGTTTCCCTTTTACGTTACTACTCGGCATTATTC |
| | | AAACTGGTAAACGATTTTGAAATCTATTCACTGGCGAAAGGCAATCCTACGGTGGTCAACTTAGATGTGTTGGT |
| | | GGATGATAGTAGTTCCCCGTATAAGTTTCGCGGCTTCTACAATTTTGGAAATATGATCGGTCAGATTCATGGTCT |
| | | CACTCACAATCAAATTAAGAATGAAGAGATCATGATTAGAAACAAGATAGCACACTTCGACATCGAAATGTTAC |
| | | TGAAAAAGCCTCTGCTGGCTCAAATTAAGTTGGATGTGCAGAGAAAAAACTTGATTAAGTTTATAGAGGATCGC |
| | | GGCGATATGAAGAGTGTATTAGGTTATGATGCTATTAATGACTTTAGCATGAAGATGGTTCATCTTCGGACAAA |
| | | AATCAAGGTAAACAGCGATAAGCTGCAAATCATGATGGGCTTATTGACTAATGTCAAAACCCCAAACGATTTTT |
| | | ACAACATCTACAAAGTCAAAGGTATAGAAAGCATTAATCAGCATCTTCTGGATGTCATTGGGGTAACAGAGTCC |
| | | GAAAAAAAGATTGAAAAAGAGATAAACGAGGGTAATAAACGCGCAGTAAAATAA |
| 286 | JW13107 | ATGGAAAATCTTAGTAAAGAACAAATAGTAGAAACTCAGGAGCAAAACAAAAGAACGTGGGGTAATGACCCTC |
| | | AGTATTTTGGGGCCTATCTGAATATGGCACGGTTAAACATCTATAATGTAAGCAATCATCTTGCCGAAAAATTTG |
| | | GTATTACCGCGATTGAGAATGAAGAGCAGATTTCAAATAGTTTCTTGGGGGATAAAGAGAATAAAGTTTTCAAG |
| | | AACAAGCAGAACCATATATTTTCCAATCTGATACGTTATCTGCCTATTGTAAAGGTTTTCGATTTCGAGAAACTC |
| | | CCCGAAGAAGAGAAAATCTCACTGAAAGATAATTTCGGTAAAGATTTTAAAGAACTCTCAGACACGCTGAAAAT |
| | | TATCTTTAGTGAAATAAACGAGTTTCGTAATGACTATTCTCACTATTTTTCTACCGAAAAAAAGAACAAACGTAA |
| | | GACAAAAGTCTCTGATAAGTTAAAGGACTTCTTAAACATTAACTATACCCGTGCTATTCAATATACAAAAAACC |
| | | GCTTTATCAACGTCTTTTCAGATAATGATTTCTGCCTGGCAGAGGAAGCGAAACTGGTCGAAAATGATAATACA |
| | | ATTACACAGGATGGTATCGTATTTTTTACCTCTATGTTTCTGGACAGAGAAAATGCTTTTCAGTTCATCTCGAAA |
| | | ATAAAAGGGCTGAAGGGCACACAAACAAAAGCGTTTAAAGCAAAACGTGAAGTCCTGATGGCCTTTTGCATCA |
| | | ATCTGCCACACGATAAGTTTGTTAGCGAAAATACAGAACAGGCATTTTCTTTAGAACTCATTAACGAACTGAAC |
| | | AAATGTCCGAAAACACTTTATAACGTGATCTCTGAAAATGAGAAACAACATTTTTTACCGAAACTTGAGGACGA |
| | | ACAAATTGATAATGTGTTGGAGAATAGCGTTCCCTATACTATTGATGATTACGAAAATTACATCGAGAACATTA |
| | | CCAAAAAAGTCCGTAATAAGAACCGCTTCTCCTATTTTGCCTTAAAATTCATCGATCAAAAGGAGCTGTTCAAA |
| | | AATTGGAAATTTCAGATTGATCTGGGTAAAGTTGTGTTGGACGAATATACCAAAACCTTACAAGGCAAAGACGA |
| | | AACGAGAACGGTTGTGGAAAATGCGAAAGCGTTTGGCAAACTTCAGCACCTGAACGACGAGGAAAATGTTCTT |
| | | AATAAAATCAACAAAAGCGAAAGCAAAACCCATTTTGAACAATTCGCTCCGCATTATAATTTTGACAATAATAA |
| | | AATCGGTATCTCCCGTAAAGAGGATAGCGCGATATTCATTTCACGCAACACAGATAAGAAAGTCAAGTTTAATC |
| | | TGAAACAACCGCTGCCTGAAGCATTTTTATCTATTCACGAACTGCCCAAAATCATCCTGTTGGAATATCTTGAAA |
| | | ATGGCAAGACGGAACAGCTGATAAACGATTTCATTGAGATCAATAATTCGAAAATCCTGAACAAAAAGTTCATT |
| | | GAATCCATTAAGAGTAAGCTGACCGATTTTAACACCTTTAACAAGCGGAGTCAGGGTCGCAAAGAGAAATCCGC |
| | | GTATAAGAAAGAAGCCTTAAAATATTTGCTCGATCGCAAAGAAAAGCTGAATCAGATATTAGCCGAATACAATC |
| | | TGAATGTGAAACAAATTCCAACCCGGATTTTAGATTATTGGTTAAACGTAGCCGATGTTCAGGAAAAAACCGCA |
| | | ATTTCCGATCGTATTAAGCTGATGAAACGCGATTGCATGGACCGGATTAAAGCGATGAAAAAGGGAAAAGCCC |
| | | CGAAAATTGGCGAAATGGCAACTTTTATTGCCAAGGATATCGTTGATATGATTATTGGGGAAGAAAAAAAGAAG |
| | | AAAATAACGAGCTTCTACTACGATAAAATGCAGGAATGTCTTGCTTTATTTGCCGACAAAGAGAAAAAAGAACT |
| | | CTTCATACACATTTGTAATAACGAACTCAAACTGAATGAATCGGATGGGCATCCATTTTTAAAGAATATTATACT |
| | | TTACGATTACCGCTATACTTCCGATTTGTATAGACGATACCTTGAAGAAAAAGCTGATAATCGCAACAATCTTAA |
| | | CAAAAACGACACGTCATGGCTGGCTAAGAATTTTTACTTCATCGAAAAAGTTGAGGTCTTCGACAAACGGCTGA |
| | | ACCGAATGGAGTTCAAACCTCAAACAGTGGTGAAAATTAAGGATAAAAGCAAAATCCCATTCACTTTACGACAG |
| | | TTAGAAAAAGAGAAATCTTCCTTTGATACGTGGTTTGAAAACATCACAAAGGGCAAAGAAACAACTGATAAAA |
| | | AGAAACCCGTAGACCTTCCGACCAACTTGTTTGATAACTATCTTAAAGAGCTTCTGATTAAAGATCTGTCGAAAA |
| | | ACAACATCAAGTTCAATCCTAAGACGAACTATAATGAATTGTTTAAGCTGTGGTGGAAAGAGTGTCGGAAAGAT |
| | | AAAACTCAGAGTTTTTACAATGCTGAGCGTGAATATTCATTTGAGGACAAAAAGCTCAATTTTAAGATCAATAC |
| | | GAAATCAAAATTTGAGGACTACATCAATCCGGAGTTCGCAGAGAAAGTGTTCAAACTTAAAAGTAGCCGCCGTG |
| | | AAAAGGAAAATAGTACTGGTCGTAAACTTCCGCCATTGATGATCAAAGATCTTGAAAAAAGCATGGCGAACAA |
| | | AATTGGAAGTATTGAGAAAGATATCAGAATATTACAGGAAGATGACTGCATGATGCTGCTGATGTTTGAAAAAC |
| | | TTGTAGACAATAATTTGGAACTGAAATTAGAGAATGTAGACACCCTCATGAACGAATCTATCGAGATAAAGCAG |
| | | AGTATTTTAGGAAAGCTCTCATTTGATGACTTTGGCGATAACGTGAACAAAAATGACCGCATAATAATTAGCAA |
| | | AAACATAACCGAGACCCGGAAACGTAAGGAATATTCAGTTCTGAAAAAATATATTTTCGATCGCAGATTGCCAG |
| | | AGCTCTTTGAGTATTTCGAAACCGAAGATATAACTATGGAAAAAATAAAGCTCGAATTGGGTACTTACAATAAA |
| | | GCAAAAGACATCGTGTTCGACTTAATTTTCGATTTGGAATCGGCTATTATTAAAAAAAACGAGAATCGAATCAA |
| | | GGAGCTGAATAAAGATAAGAATGGAAACATTCAGACGGGAAATATTCAGCATAAACCTTATCTGTTTTGGCTGT |
| | | TGGAAAAGAATCTGATTACAGAACAGGAGTTTACCTTTCTGAATATGGTTCGTAATACTTTTTCGCATAACCAGT |
| | | TTCCGCAGAAGAAAACAATGGAGCTGTTTATAAAGAATTGGAATAAAAACGAATACGCAAAGCAAATTCTGAC |
| | | GGTTTACACTGAAAAAATAAACAATCTTATCGAGAAAGTGATTGACATTTGA |
| 287 | JW13108 | ATGGAATCTTTCCCTGATTACAAACCGCTGTTTGCTGCCTATCTCAATATGGCCCGTAACAATTGTTTTATCGCGC |
| | | TGAGTCATATAGCAGAAAAAGCAAACAAAGGCGGCGGTATGAATGAAGATGCGATGCAGGAATGTAGTGTCAT |
| | | AACGCTCTTAGCCAATAATAAAAAGCCAGACGAGTCTTTAAAGGTCGAAGAAATGATCAAATATCATTTTCCAT |
| | | TCACCAAATCAACAAAGCGGATTGATCCGGTTCCTTCTGACTATGCGGGCCTCATCATTTCCTTTATTAAGCAGT |
| | | TGAACGAATTAAGAAATTTCCATACTCACTACTTGCATCGTCCGTATGCTACTGATGCTAATGTTATTGCGCAGT |
| | | TACAGTTTCTGTTCGATACTTCACGCAGAGAGGTGCGTAAGCGTTTTGCACTGAAAGAAGAGGATGTGAAACAC |
| | | CTCGTGCGTAAAACAATTACAATTATCAATGGCGAGAAAGTTGTAGTGGAAAAAGACAACTATCCTTACGCGTT |
| | | CGTAAACAAAGAGGGTCAATTCACAACAAAGGGCATAGCCTTTTTTATCACTCTCTTCCTTGAGAAAAAACATG |
| | | CATATCTGTTTCTCAAACAGCTGCGTGGATTCAAAAATAGTAGCAGCAAACAGAACAAAGCCACATTAGAAACC |
| | | TACTGCTTTTATCATATTCGTGTGCCTCAGCCAAGATTACAAAGCCAGGATACTAAAGTTGGGTATCTGCTGGAC |
| | | GTTTTGGAAGAATTAAAACGGTGTCCTAAAGAACTTTATTCTCATCTGTCAGAACACGACCGAATGCGTTTTACG |
| | | TTTGTTAACGACATTGATGCTACGGACGAGCAGCCGGAAAGCATTTTGAAACGTAACAAAGATCGTTTCGCGCA |
| | | TCTTGCATTGCGATATTTTGAGCATACTGGTAGTTTGAAAGAACTGCAGTTTTATTACGACCTGGGTAAGTATTA |
| | | TTTCAAAGTGTACGAAAAATCTGTCGATAGCGAGATTCGGCTTCGCCGCCTTCATAAGTACATGAAAGGATTTG |
| | | GTCGTTTGAATGACTTTGGCCTTGAAAACATGCCGCCCTTATGGCAACAATTGGTTAAGTACACCGACCAGTTAT |
| | | CTGAGGGGTATGCGGAACCTTATATTGTTTACACCATACCGCATTATCACATCGTCAATAATCAAATTGGTATCT |
| | | GGATTAACAATGGGTTTATACCGCCGCTGCTGTCGCCGGTGAGTAGTCCAGAACCACAGATGTGGCTCAGTATC |
| | | TATGAGCTGCCAGCGATAATGTTTTATCATTTGCTGACGAAAAAACGAAAAGAGTACAAAAGCGTCCTGCAGAT |
| | | CCTGTCTAACCACCGTAAAGCGATTCATCTTCTTTTCAAAGATATTAAGGATTCCAAACTGCTCCCCGATGCAGT |
| | | GGAAACCTTTGAGGCCGAATTAAGACAGCGGGGTTTACATCGCAACCACATCCCGAAGCCCTTGCTGGAATATC |
| | | TGCTGAATGAAAGCCCAGGAACGCCCGTAGCCGCCAAAGCAGAAAATCGCCTGATCAAGCTGCAAAACGAGAC |
| | | AAGCTTTCTGTTTGATAAGATCGATATTGATCTGGAAAAATCGAAAGAAAAGGCTGGGGGCAAATATTATAAGC |
| | | CGATTCAAACAGGTAAAATCGCTCAGTTTTTGGCAAAAGATATGCTGTTTTTCCAACCGCCGATTAACAAGCTGA |
| | | AAGGAAAAGCAACCGGTACTCTCTTTCAGGTTCTGCAGGCCCGTCTTGCTTTTTATGGCAGAGATAAACATCTTC |
| | | TCGAAGATATCTATCGCGAGTGTAATTTGATCCAATCAAACAACCCACACCCGTTTCTGCAAGGACTTCCTATTA |
| | | CGGATAGCCTGATCGATTATTATCGCAATTATCTTGTCCGGCGAAACACGTATCTTGAAAAATGCCTCAAAAAAT |
| | | GCATGCAGGGGAAAACCGGAAGCCAAGAGCTTCACTTTCTGCATCTTGGCGAACGAGAAAAACGCGCAGGTAA |
| | | TGATTATTATAAAGGGCTTGCGGGGAAATTCATCAAGATGCCATTAAACCTGCCCCGTGGTCTGTTTATGGAAG |
| | | AACTTCTGAAGGTTTTTTCAGCTGAGAATAAAACAATGCAGGACATCGCGGATAATTCGCGCACGAATACGGCT |
| | | TTTCTTATTAAAGAATACCTGAAAAACGTACTGGACGATGATACACAGGATTTCTACAACTGGAAAAGAACTTA |
| | | TGCAGTATTCAATAAGTACTACGACAATCGCAAACCCCAGAGCCGGGATAGTCTGATTGAACGATACTTCGATA |
| | | CCTCTGTGCTGTCAACCGAGGCGGAAAAAATTAAACTTGAAATTGTAAAAAAACAACGCGTCGATATAGAGGCT |
| | | TCCGAAAACATTAAACGTAATTATCGCTACTATAGTGAGAACGAAAAGCAGATCAGACACACCAAATCCTGCGA |
| | | CATAGTTTTATTTCTGATAATTCAAGACCTGTTCCCTTCGCTCGAGCAATCTGTATTCAAAGGTTTAGCAATTAAT |
| | | CAGTTAAAGCTGTCCGATATTTTACCTGATGCGGAAAAAGGCATTCTCTCATTGCCAATTGAGATACGCCTTCCG |
| | | CTGTATAACAAGTTCATCATCCAGCGGGGCATCAAACTGAAAAATTACGGTGACTTCCGTCGTTTTTTAAAAGAC |
| | | CGCCGGTTACAGTCGCTGTTACCTTATTTTGAGGAAGAATCCATTGACAGAGTGATAATTGAAAAGGAACTGCA |
| | | GGATTATGAAATAGCACGTATAGAGATTCATCGGCTTGTATTGGAGTTTGAAAAAAAGGTTATAACCGAGAATA |
| | | AGATCACATACGAAAACTCCGAGATTAATTATGTTGATCATGAAAAAGTGCTGACCGCTTTCTTTCAGAATGAG |
| | | CCTTCCTCTAATAAAAAAGAGCTGATACGCGACATTAGAAATAAGTTCTCGCACAATGAATATCCGGATGCAAA |
| | | TAAGTTTAAAGGATTAATTGAATCAGATTCACTTAATACGACTGTCGCCAAAAAGTTTATCCAAATAGCCAAGC |
| | | AGTACTACCAGTTTGAAATTGCCCCCGCCCCGCCCTTGTTAGTGTTATCACCAACCAGTACCACTATTTTGTAA |
| 288 | JW13110 | ATGTCCATAATGAATATTATAACCGTTTTAAACCATATTCACCAGATCGCTGGGTTAAAACGAAATGAACTTGGC |
| | | GATGATGATAACATCTATTCACACCCCGTGCTCAAATACATTGCCCCTGAAAATAATAGCACCACGTTGCCGGA |
| | | GAAACGCGATCTGATAAAAGAGCTGCTTTTGGAGTATCTGCCCTTTCTTGAAGCAATGAAAGATTGTTACAAAG |
| | | AGGTCAAAGTAGAGAAAGATAAACGAAACAATAAGTATGTGAAAGACAAAAAACAGGTCTTTATCCGCAATTA |
| | | CTCGGATATCTATTGTATCTACTGGACTCTGTACAACATCTTCTCCACCTTGATAAACTATCGTGATAAAAGTTGT |
| | | CATTATCGTTTTATCCCTAGCAAAGACCAAGAAGAATATTCCAAACGCACAGAGTATGATACAGTACGTATAAT |
| | | AAACAAATATTACACATACGCGTTACGGAAAACCAAAGAAAAGTACGGTTACACGACCGAACAACTCTCTTTTA |
| | | TTCAAAATAACCGGTATACAGATGACAAGAAGGTGAACTTTAATTTCTTTTTATCGATGCAGTCCTCGAATAATA |
| | | ATCTTTCGCATGTTGGCGTTGCACAGCTGATCTGTCTGTTTTTACAAAAGAAAGATGTTGATAATTTCTTGGAGA |
| | | ATTTGCCAATCTATGACAATCGTTATTGCCTGGGCATTGATGAACACATGATAATCAAACGTTCTTTAGCCTTAC |
| | | ATAGTATAAAGCTGCCAAAAGAGCGCCTTAACAGTGAGAAAAAAGATATGGCAATTGCAATGGATATGCTCAA |
| | | TGAATTAAAGCGTTGCCCAAAAGAATTGTTCAATACCTTACCATTTGATAAACAGGAACGCTTTCGTATTATCTC |
| | | ATCAGACCATAAAGAGGTGTTGCTGATGCGCAGCACCGATAGATTCGTCCCTCTGATTCTGCAATATATCGACTG |
| | | CAACAAAAAATTCAAGGATATCCGCTTTCATGTCAATATGGGTAAACTGAGATATTTGTTCTCCCCTAATAAACT |
| | | GTGCATCGACAATCAGGAGCGTGTGCGCATGTTGGAACATCTGTTAAACGGCTATGGACGGATAGATGAAGTGG |
| | | AATATCTGCGCAAGGCTCCTAACGGTACGTTCGGCAATACCAATATACCTATAAAGGGGTTTGGCGAAATTGAA |
| | | CGTGACTCTGCTGACAGCAACAACTATCCGTACATCGTAGACACACATACACACTACATTCTGAATAATAATAA |
| | | AGTAGAGTTCTGCTTTTGCGAAGATAGTAAGAATAAGAATGTCTTCCCAACTATTGAAAAGGAAATAAACGGAA |
| | | AATGGTATGTTGAGAAAACTGCCCCGCATTGCCGTATTAGTTTCTTTGAAATTCCGGCAATGGCGTTCCACATGT |
| | | TTCTGTGTGGAAGCGAAAAAACCGAAAAGCTCATCAAGGATGTTTACAATAACTATATCAGCCTCTTTAATGCG |
| | | TTAAAGAATGGCGCCTTGACCGAAGAGAATTATAAAGATTTTAACATACCGGAATGTGACATGCCCAAAAAGAT |
| | | ACTGGATTTTCTGAAAGGCAAAGCCGAAGGGAAATCAATTGATGGTTATGTAAAGAACATTTTAAAAAAGGAG |
| | | CTGAAAGATACGCGGAAACGAATTGAACAGGCGCCATCGGAAAAGGACCAGATGCCCTCAAAACGTAATAAGA |
| | | TAGGTACCAAAAGTTACAAAAGAGTCCTCACGGGGAAACTTGCCGACTTTCTGGCCAAAGATATTGTGAAATAT |
| | | CAGCCAACGCTGTGCGCAGGGGATGATTATGGCATTGACCGTATGACGGGTATGAACTATCGGATTATGCAAGC |
| | | GACCATTGCGACCTATAATGACGATGATGATTTTGACACGTTCTACAATGTGTTTAAAATGGCGGGTCTGGTTGA |
| | | TGGTGACGAGCGTAAAAACCATCCGTTCTTAAAAAAAGTTCTTCTTAGTAAACCCCAGAATACGGTAGCTTTTA |
| | | ACATCAAATATCTCAAAGAAAAAGAGATCTACCTGCGCAATATTGAGAAAAAAAATATCATTCCGCCGTTTGTT |
| | | AACCGAGAGAGAAATAAGTGGTGCGAACGCGACTGTGAATATTACAGAATTCTGGGAGAAAATCTGCTTGACTT |
| | | ACCGATTGAACTGCCACGTCAGATGTTCTACAAAGATATTCGGGCTCAACTTGAACCGTTAATGCCTGAGATTG |
| | | ATTTTAGCCAAACTAATACTACATATCTGATTGCTGAATATCTGAAACGCGTTCGTGGTGATGATTTTCAGGAGT |
| | | TCTATTCTTGGGAAAGAAATTACAGATATATCGACTTTCTGAAATGTATCCGGGATGATAATGGACGCATTTGTG |
| | | CGCAATATACAACGACAGAGGAACGGGAATCTTTATGGGAAGATAGCAAAAACCGGACCGCTGAATATAAACG |
| | | ATCTGAGGAGCAGAAGAATGAAAAAAAAGATAAAAACTTTGAAGACAGTTTGGAAGAACGTATATCATCAAGC |
| | | CACATCAAGTTTCAGAATACTGAAAAACATATTCGTAGATACAAAGTCCAGGATGCTCTGTTGTTTATGATTGCG |
| | | GAAAAACAGTTTGGGAAGTATCTTGATGATATTAAAGCCAAGAACTTTAAGCTTAAGGATATTATGCCCGACAC |
| | | GGACAAAGGTATTCTCTCTGAGACAATGCCTATTGATTTCACTTTCACAAAAAAGAACATTAAGTATAACATCC |
| | | ACGCTAAAAATCTCAAGTTTAAAGAGTACGGTGATCTTTTCATATTGGCAAATGACAAACGCCTGGAATCATTG |
| | | TGGAAAATCCTGGCGACCGACACTGTGAACAAAGAAGAAATTTCTGAAGAGTTTGCAAAATATGATTTTTGTCG |
| | | TCCGGACATTACTAAACTGGTTCTTGATTTTGAAAAAGATGTACTTGAGAAGTATCCTGAGATTATTGAAGAGGC |
| | | AGAAAAGGAGTATAAGAAATTTGACTTCAAGTTCATCTTAGATTGGCTGATTAACAAGAAAAAACTCAACTACG |
| | | GAATACGCAACAAGCTGTTACTGATCAGAAATGCCTTTAATCATAACTCCTATCCGGATAAAAAATATGCAGAG |
| | | GTGCGAGAATTACCGGAAATTGCAACTAATCTGAAAAAAACATTTGACGAAAACGCCAAAAAATACGAATAA |
| 289 | JW13112 | ATGCGCATTGTCAAACCTTATGGACGCAGTCATGTTGAGCCAGCCGCTACAGGTAGCACTCGTCGTGTACTGCG |
| | | CTTGCGTCCGCCGAACGACAAAGATGCCAAAGATATCGAAGCATTTGCACGCTCTCATGAAGAGCTTGTGATTG |
| | | CGCAATGGATATCAGCGCTCGACAAAATTGCAACCAAGCCTTCCGGTAAAGACGGTGCCAGCGATGCGCAACG |
| | | CGCTTTTCGGGAACGGTTAGGAAACGCGGCCTGGACGCTGCTGGAAACGAAAGGGCTGCTTCCCGGCTTGGTGA |
| | | CACGGCGTGAACACTTAACCATGCTGTGGCGTGCAAAAGTCCATCCATACGGATCGACACCATACCGGCCGAAA |
| | | AGAGGTAAACCTGCACCTTCGCTGAAAGGGCGTCGATATGAGCGTTTCGCCGGCCAGCTCGAAGTGGATAAGGT |
| | | AGATGCAGCAGCAATCGCGGCGAGCATTCATGCACATCTTCATGGGACAGCTGTACCAGCGGTCCGCCCCGGAT |
| | | TGATAGCCACTCGTGCACGCTCAATTGCGGAGAACGTGCTTCGCCCACCGCAGCGGCTGCCTAATGAGGAACGG |
| | | TCGTGGTCCGATGACGATGAGCGAGCCTATGCTGCTGCAGGCGATGTAGCCCGCGAAATTCAAGAGTTAGCGAT |
| | | CGCAATCGAGGAAGACGCAAGCCAGCGGCGCCGTGGAAAGCCGCTTACCCGTAGCCCATTTGCTTTACGTGATG |
| | | CAGCTCCCGCTTTGCGCGGCCAATATGGTAGATTATTAGTGGATGCGTCGGGTGATGCCCTGCCTATTGCTCAGG |
| | | CGCGCGCTGCGTTTCCTGGCTTATTTGGCCTGCACATGGCGGTGCGAGACTGTTATTCGCGCATTCTGAAAGATC |
| | | ATAGAAAAAGTTCTGTCGTACGCATCTTGCCGCGCGATATGGAAGAGTTGCTCCGGCTGGTTGGGGCAAAACTG |
| | | ACTAATCGCGATCTCAATGCGATGGTAAGACTGGGGAAGGTTATTCATTACCAGTCGTCTCTTGGCGTCGAAGA |
| | | TAGTCCTGAAAATGTGGCGGCCAATTGGCCCGCAGACGTGTCTGGAAGCGAGTTTTGGACCAGTGCCGGGCAGG |
| | | CTCAGATTAAACGCAACGAGGCTTTTGTACGCGTATGGAGACATGTCCTGGCTCTGGCCCAAAGAACTTTAACG |
| | | GATTGGATCGATCCCCAGGGTAAGACACAAGGTGATATTCTGTTAGCGAATCCGCCCTTTAATCGTGACGCCTAT |
| | | AATCGCAAATCTGATCTTCTGTTCGGCAAAAGAGCCTCACTTTTTAAGGGCGTTGAGACATTTGAAAATGTCGTG |
| | | CTGACACTTGCTCGCCGTGAAACCGCAGCCCTGCGTCATGGTTCTTTTCATTTCAAAGGATTAGGCGGATTCACC |
| | | AAGGTTTTGGCCGAAGTTCCAAAGACCGACGCATCCGTGCTTGCCGCGGTTCGGACGTTATGGGAAGATGATTT |
| | | GAGTGAACGAGCACAGCGCCTGCGTCAGACGCTGCGCGGCGCTCATGTAGAGTATTTCCTCGACGAAGCGCAGG |
| | | GTAGACGACTTTTATCTGCGGTGCTCGCAGATTCAGGACCGGCAACCTTGCCGCTGCCACGCTTTAGACGTATTC |
| | | TGCAGCGAGCACATAATGCATGGGCAAAGGAAACCGATGGTCCCGGTTTACCTGAGCCTGCAAACCGCATGGA |
| | | CATTAAGACATATCCGGCCCTGCATTGCCGGTATGTAGCTCTGAAACATGTTTACGAACGGCCATTTCGGCATTG |
| | | GCTTGAACAGCGAGACGCCGCGTCACTCAATCGTCACATTGATCGTGCGGTTGATCGAGGTACTAAAGCGGCTC |
| | | GTGATCTGAACGCGGGCGACGATGAAGACCGTCGTCGTATTATAGTCGCTCGTGCAGCCTCTCTCGGTCGTTTAG |
| | | CCGAAGATGATAATGTTCAGGATTTTTTCTTCGAGCTGTCAGCCGAAACAGCCGGCGAAATGCGTGTTCAACGT |
| | | GGATATGCAAGCGATGCCGAGGCTGCTCGGGAACAGGCTGGGTACATCGAGGACCTGAAATGCGATGTGTTAG |
| | | CGCTCACCTTTGCCGACTATCTGAAAGCTGCCGGGCTGGATTTCATACGTATCTTGGCGCCGGAGACGCCGAGA |
| | | CCCGACAATGCAGCATATAATCTCGATGCTATAGAGGCACCGGAAATTGATACTCCGGGTGAAGATTGGCAGCG |
| | | GGTTCTCTATTTTATCTTACACCTCGTGCCGGTGGAAGATGTTGGTCGGTTGCTGCACCAGACTAGAAAATGGGA |
| | | AATTCTGGCCGGGGATGAAACGCTTTCCCCCTCAGTGCGGGACCGACTTCGGGGGATTCAATTGGCTCTGGAAT |
| | | TGTACTTAGACATGCATGACGCCAAATTCGAAGGTGGCGCCGCACTCGTTGGTACCGGCGCATTCAAGGATCTC |
| | | TTTGAAACTGCACGTATATTCGACGACATTTTTCCTTCTCAGCCCGGATCCGCGCAGACTGCGGATGACGAACGC |
| | | CGCGTGCCAAAACGGGGTTTGTGTGAAATTATGCGCTTCGGTCACCGTCCGGCACTTCAGCCGATCTTTAGCCGC |
| | | TACCGCGTCACCGAACGTGAAGTTGAAGAAGTCCGCGCCATGGAATGGCCGGTGGACGGTACCTCAGAAATAG |
| | | CCAGACTGCAGGACCGTCGCGAAAGACTGCACAAAGGATGGACGGAAAACAAAAGTGGGCTTTCCGGTGATGA |
| | | TCTGAGAGCCTATGTCGCTGCGTTGCGTGAGGTTGCAGCTCATCGCCACCTCGCCGCGCATGTCACCCTGACAAA |
| | | CCACGTGCGTCTTCACCGTCTTTTGATGGCAGTGCTCGGGCGGCTGGTCGATTATTCCGGCTTATGGGAGCGCGA |
| | | TCTCTACTTTGTAACCCTGGCTTTAATACACCGTGCTGGCAGACGGCCGGCGGATGTTTTACCAGCAGATGGGTT |
| | | ACGATTTCTGGCCAATGGTCAGATTGTTGAGGCAGTTCGACGACTTGAGCCAGACCTGCAAAGCCGTCTGCTCG |
| | | ACTGTTTGGGCGATATCGATAAAAACGGGAAAGTACGCATCCGTAACCGCTTTGCACACTTCAATATGCTGCAA |
| | | CCTGGAAACTTACCTGTTGATCTGACTCGTTGCGTTAACGATGCGCGCGAACTGATGGCCTACGATCGTAAACTC |
| | | AGAAATGCGGTGACTCAATCAGTTAAAGAACTGTTAGCGCGCGAAGGTCTGATGTTAGGCTGGGCTATGGGCGC |
| | | TGATCATCGCCTGAGTGCTGCGACACTGGCGACGAGACAGGCGCAACACCTGGGTAAAACGCGCCGCGGCGAA |
| | | AAGGGTTGGCATGAGCGTAAGTTACGTCCGATACCAATCCTTGAGGACCTGCATGGCCGTCGCTTTGTTGATATG |
| | | GCTGCCGCCTTGTTTGGCGGAACCAGACTGGAACCGGAGCGCAGCCTTCTCGATCATCCTCTGGACCGCATCGA |
| | | GTGGAACCCGACGCCGAAAAATCGGTATGGGAGTCGCCCTACAAGTGGACAAGGTCGAAGAAATGTCGTACCA |
| | | CGATAA |
| 290 | JW13113 | ATGGAAAACCAGGAGCTGAAGACACGCACCATGAAAAGCGAACCATATTATTATGGGGCCTTCCTGAACATGG |
| | | CCCGTTTAAACATTTTCAACATTAGCAACCACTTATCGAATAAACTGAACATTCTGCCCACCCTGTCCAGTGAAG |
| | | AGCATATTGCTAATGCGTTTTTTACCGACAAAAATACCAAAATAAAATGGGAGCATACGTACGACATTCTGAGA |
| | | CGTTTTATCCCCATCGTTAAAGTTTTCGATACGGAATCTTTGCCAAAAGGAGAAGTCGGTAACAATACGGGAAA |
| | | AGACTTCTCCAAAATGTCGGATACCTTAAAAATTATTTTTAAAGAGCTTAACGAGTTTCGCAATGATTACTCTCA |
| | | CTATTACTCGACCGAAAAAGAGGATAAACGCAAAATTACTATTTCAGATGAACTGGCCAATTTTTTAAACGAGA |
| | | ACTTTAAACGCGCCATTGCTTATACCAAAAAACGTTTTAAAGGCGTCTTTACTGAGAAAGATTTCGAGCTTGCCA |
| | | ACAATATCCAATTATTCAATAAGGATAAAGAAATTACCGAAAAAGGACTTGCGTTTCTCACGTCAATATTTTTAG |
| | | AACGCGAGTACGCATTTCAGTTTATCAGCAAAATAGAAGGACTGAAAGGGACCCAGAAATCAGAATATCGAGC |
| | | GACACGTGAAGTTTTTATGGCATATTGTGTTAATTTGCCGCATGATAAGTTTATTAGTGAAGATGCAAAGCAGTC |
| | | ATTCTCTCTTGATATAATTAGTGAACTGAACCGTTGTCCCCAAACCTTGTTTAATGTAATCACGGAAAAGGAACA |
| | | GGAAAAATTTCGGCCGACTATAAACCAGCAGGAGAAAAACAACATTATAAACAATAGTGTTCCGTATGATATCG |
| | | AGGATTACGAAGAGTATGTAAACAGTATTACTAAGAAGATTCGGTATGATAATCGTTTTCCATTTTTTGCGCTGA |
| | | AATTCATAGACGAAACTCAAGTCTTTGAAAAAATACGTTTCCAGATCGACCTCGGGGAAATACTGCTGGATGAA |
| | | TATACGAAGCAGTTGGCAAATAATGAGGAAAAAAGACAAGTAGTGCAAAATGCGAAAGCGTTTGGCCGCCTGA |
| | | ACGATTTCATTGACGAGAATAATGTGCTGGAGAATATTAATAAGCAGAATGGCTCTGCTTCGTTTATCCAGTATG |
| | | CCCCGAATTATAATTTTGATAACAATAAAATCGGTATCGACACAACGGGTAAACGGATTATGCCTATACTCACT |
| | | AAGCAGACAGACAATAACAAGAAAGTTAAAAACAAGTTAAAACAACCTCTGCCGAAAGCGTTTCTGTCAATCC |
| | | ATGAATTGCCTAAAATTATTCTGCTTGAGTACCTGGAAAAAGGTAAGGCAGAAAAGTTGATTAATGATTTTTTAT |
| | | TGATCAACGAATCCCAGCTGCTTAATTACAAATATATCGAGGAAATCAAAAACAAACTGAACAATTTCGACGTT |
| | | TTTCAGAAACGGAGTCAACGTAAAAAACTGCAGACAGCTTATAACAAGACCAACATAGAGGAATTACAGAGCC |
| | | GTAAAGAAGAATTAAACAAAATCCTGAAAGAATACAAGCTTAATGATAAGCAGATTCCGACCCGTATTCTGGA |
| | | ATATTGGCTGAATATCGAAGATGTAACGCCAAATGAGGCTATTTCTGATCGCATAAAACTTATGAAAAGAGACT |
| | | GTGTGGATCGGCTGCGCGACATAAAAAAAGGTAAAGCCCCGAAAATAGGAGAAATGGCCACGTTCATTGCTAA |
| | | AGATATTGTGGATATGATTATTAGCAAAGATATCAAACAGAAGATCTCATCATTCTACTATGATAAAATCCAGG |
| | | AATGCTTAGCTCTCTACAACGTTTCCGAAAAACGGGATTTGTTTTTGACAATTTGCAATGAACTGCGATTGCTGG |
| | | ATGCCGATAAAGGCCACCCGTTTCTGAAGAACATCAACTTGAATAGAATCAACTATACATCCGATTTTTATGTCA |
| | | AGTACCTGCAGGAAAAGGGGCATAAACTTATTAAAGAGACGAACTATAGAACAGGTAAGCTCGTGGAGAAAGA |
| | | TAAAAGTTGGATGTTCCTTAACTTTTATTATCTCAAGAAGAATGAGACACTTAATAAGATGATGACCATCGTGCA |
| | | GCTCCCAGATGATAAAAGCAAACTGCCTTTTACTATTGCACAATTAGACAAACCCAAGAATACTTTCGAAGAAT |
| | | GGATTAATAACATTACTAAGGGTAAGACCAAGACAGATAAGGAAAAACCTATTGACTTGCCTACAAATATTTTT |
| | | GACAAGGAGATCGAGGAGATCTTGAAAAACAAGCTCTCGGAAGAGAAAATTGCGTTTACGGAAAATGCAAATT |
| | | ATAATCAGCTGTTTAAACTTTGGTGGACCGATTGCCGCAAAGACAATGTGCAGAAATTTTATGATGCTGAACGT |
| | | GAATATGTCATCTATGATGAACATGTGAAATTCATTCCAAATACAAAACCTAAGTTTGAAAACTACTATAACGA |
| | | AAGTTTTCCGATAGTTTTAACCCGTTTAAAAAGAGATCGCGAAGAAGCACGTAAGCTTAATCGCAAACTCCCGC |
| | | CCATTGAAAAATCCCAAGTTGAAAAAGTATTCAAACAAGCGATCGGTTCTACCGAAAAAGAAATTCGACTGTTG |
| | | CAGGAAGAAGATCGTATAATGCTGCTGATGTTCGAACAACTGTTAGAGACTGACAATAATCTTAACCTGAAATT |
| | | AAACAATGCAGAAAGCTTGCTGAATGAGCAAATCACTATTAAAGAGAAGATCAGCCTTAAACTTTCATTCAATG |
| | | AGCAAGAGGACAAAAAAGAGATAATAAAGACGATCATTGACAAGCGTAAGCGCAAAGACTTTTCTATTCTCCG |
| | | AAAATTCAAGTATGATAAACGGTTACCAGAATTATGTGAATATTTCGAAACAGATGATATAAGCCATTCTGATC |
| | | TGAAGAAAGAGTTAGACGAATATAATAAAGCAAAGGAACAAATTTTGGCTAATGCCTTTATGTTAGAGAAAACT |
| | | ATTATCGAAAAAGACAAAGATGGGATAAAAGCCCTGTTCCTTAATGACAATGGCGAAAAAAAATACGGTAATG |
| | | TCCAGCATAAACCTTACCTGACGTGGCTGAAAAATAAAGGCCTCATTAACGATAACGAGTACTTGTTCATAAAC |
| | | ATGGTAAGAAATACATTTTCCCACAATCAGTTTCCGCAAAAGCGAACTATTCAGATTTTCATAGACAAAAATCG |
| | | CGGCAATCCGATAACCTTTGCAGAGACAATTAAGAATGCGTACAATAAAAAAATCGAGGAAATTATCGTGAAA |
| | | ATCAAATAA |
| 291 | JW13114 | ATGGAAACACAGGAAAATGAATTATTTGAAAACCGTCGGCGAACATTAGCGGATGATCCACAGTATTTCGGTGC |
| | | GTACCTTAACCTTGCCCGCCTGAATATTTTCTCAATTAACAACTATCTGGCAAAAGAGTTTCAACAGACGGAACT |
| | | GCGTGATGACGAACTTATCCAAAATGGCTTTATTTGTATTCCAAGTCAGAAGAACCGGAATGATAATAAGCTGT |
| | | TCGAGCGCCTTATTCGCCTGATGCCGGTTATCAAACAGTTTGATGCTGCACGACTGCCTAATGAAGAGCAGAAA |
| | | AGCGGACAAGCTGAAGGTAAAGATATAAAGACTATGGCGAAAACGCTGAAAGCACTTTTCAAAGATATGCAGG |
| | | AGTTTCGGAATGATTATACCCATTTTTATGCTACCGACAAACAGGAGTGCCGCAAACTGACTGTTTCATCCGAAA |
| | | CGGCAACGTTTTTAAAAGCGAGTTTTGCCCGTGCGATCGAATATAGTAAAGTCCGGTTCAAGGATGTACTGGCT |
| | | GAAGATGATTATAGCTTAGTAGCAGGCAAAGAGCTGGTGGAAGATGAAAATACGATTACCACCGAAGGGCTGG |
| | | TTTTTTTTACAGCTTTGTTTCTCGACCGCGAAAATGCGTTTCAGTTTATTGGCAAAGTGCAGGGGTTAAAGGGGA |
| | | CGCAATTCAAATCGTTTATTGCGACGCGTGAAGTGCTGATGGCCTACTGTGTACGTTTGCCTCATGATAAACTGA |
| | | TTTCCGATAATCCGATTCAGGCGCTGTTATTCGATATACTGAACGAACTGAACAAATGTCCAAAAAAGCTGTAC |
| | | AATGTATTGTCTGAAGAAGTGAAAAAAGAGTTCAGACCGGAATTATCCGAGAGCGGTATTGAAAACATGTTGGC |
| | | AAACAGTTTCAACGACGAAGAGCGGGAAAAGTTACTCGATGAGATTGAGTACAAAGAGTATATCGAACAACTG |
| | | ACAATCCGTGTGCGCCATAGTAATCGTTTTCACTATTTTGCTATGCGTTTTATCGACGAACAGGAGCTTTGGCCC |
| | | GGCATCCGTTTTCAGATCGATCTTGGCAAATACACCCTTACACAATACGACAAACAGGTCCTCGGCGAATCAAT |
| | | TCCTCGGTCTGTGGTAGAGAATACCCGGGCCTTCGGACGTTTAAGCAACTTTCTGACGGCAGATCCCGATTCGCT |
| | | GGAACAGCGCATCGATGTTGACCATCGGGCGAACGGATTCGAACAGTTTGCGCCGCACTATAATGCCGACATTA |
| | | ATAAAATAGGCATTGCCTTCACCGAGAAAGAACAGTTACCTTATGTTGTGCCGACAAAAAATGCAGAAACTAAC |
| | | CGTGTAAAATACTCCTTCAAACAGCCTCAACCAGATGCTTTTATCTCACTGCATGAATTAGCGAAAGCAATTTTG |
| | | CTCGAACAGTTGCAACCGGGTGAATCAGAAAAACTCATCCGTGCATTTTGCTCTACTGCGGAAAATAAGCTCTTT |
| | | AATCTTGAATTCATTGAATCCATCAAATGGAAGTTACCCGACTGGAAACCATTTCAGAAACAGTGTGACACTAA |
| | | AAAAAGCCGTGCTTATATCACAAAGAAAAAGATGTTCGCCTATGCACCAAGAGACAGAAAGAAACTGTTAGAT |
| | | GAGTTATTGGCTCCGTACGGTTTGAACTGGAAACAGGTGCCAGAAAGAATACTCAATTATTGGCTCAACATTGA |
| | | AGAGGTACACGATGCCCGTTCTATAAAGGAGCGCATTTTACTTATGAAACGCGATGGATTAAAACGACTGAAGC |
| | | AGTTAGAAAAACATCGGAAAGATCCGTCTATACGCATTCCGAAGGTGGGCGAAATGGCCACCTTCCTTGCCCGC |
| | | GACATAGTGGATATGATGATTGATAAGACAAAGAAGCAGAAAATAACCTCAATCTATTACGATAAAATGCAGG |
| | | AGTGTCTCGCCTTTTATGCCGATGCCGAGAAAAAGCGACAGTTTATTGGTTTGGTCCGCGAGCTTAATCTCAATG |
| | | CACCTGACGGCCATCCTTTCCTGAAGAATCTTAATCTCGAATCAATCCGTAAAACATCGGAGTTTTACAAACTCT |
| | | ATCTGATGGAAAAGGTTGAGAAAATGATAGAGAATGGGTACAAAAGCAATGGAGATAAAAGATTCAAAAATAT |
| | | CGCATGGATAATTCGGGTCTTTGAAGAAAAACAAAAAAACGAGAAAAACGGTAAAGATGACACTATCTATCGA |
| | | ATTCCAAATAATAGAAATGACATTCCCTATACTATACGGCAATGGGAAAAAGAGACTTCAACACTCGAAGAGTG |
| | | GCTTCACACTATCAATCATGGTAAACAGGAAGGCGACCGTAAACGCCCGATCGATTTGCCAACCGACCTGTTTG |
| | | ATGCTCGTCTGTGCGAACTGATGAAAGGGCAAATGCAAGAAAACGGACAGACATTTGCAGATACAGCCAAATA |
| | | TAACGAATTGCTGAAGCAGTGGTGGTCTCTGCGTGAAGATGAAACGCAACCGTTCTATAACGGTGCTCGTCATT |
| | | ATGTCATCAAAGAAGAGGCTGTCTGCTTTATGCCAGGTGCACAGCCCCGGCTGACTTCGTGCTATGCCGATGCG |
| | | GTTGAAGAAGCATATAATAGATTAAGTGCCGAACGTAATATTGCAAGACGGCACAACAAACAATTGCCTCTGAT |
| | | ACAGAAGCAAAAGGTGGAGAAGGTTTTCAAACATACGCTGTACGACACCGAGCGCGAGATACGCCTCTTAGGC |
| | | GAGCAAGACCGCATTTTGTTGCTGATGGCCGAACAGCTCACTCCGGGTAGAGAAACCCTGAAACTGAAAACCGT |
| | | TAGTGAAGAACTGACGCGACCTCGCGAAATTAAAAAGGAATTTCCTTATATCTCCAATTATAATGCGCAGGGTG |
| | | AGAAACTGCCGAAAGATGAGCCAAAACCGAAAATAAAAGTGACGGTAACCGGCATTATGCAGCTGAAAAATAG |
| | | CAACGATCTTAACCGCTTTGCCCATGACCGACGTATGCCTGAGCTGGTAGCCTATCTGCCCAATGCGACAATAG |
| | | ATGCTGATACCTTACGCTACGAATTGATGGATTATAATAAGGCGCGGCTCACTGTTTTTGACGCAGTGTTCAGAC |
| | | TTGAAAAGGCAATTATCGACCAAGACCGCGAGGGAGTTATTACTCTTTTTACCGATGAGCAGGGTGAAGCGAAG |
| | | CAGGGTCGTATGCAACACAAGCCCTATCTGCAATGGTTACTTAATAAAGGGATAATTACCCAGCCGGAAAGCTC |
| | | CTACCTGAACATGCTGCGTAATAGTTTTTCTCACAACCAGGTTCCGCATATTGCGGTTGCTCTCAACGGTACGGA |
| | | AGTtGTCCCGGTCAAAAACATTGCATCTACCATTGCTGGGGCTTACAAACAAAAAATCGAGACAATTATTGGGA |
| | | AGATCGGATAA |
| 292 | JW13115 | ATGAATAATAACATACTGGAACAGTCAACGTCAGGGTCAGAAAAACGAGCAAGCACCCGCACTCTGGAAGCTG |
| | | ACCCACAGTATTTTGGCGTATACATAAACATGGCGCGCCATAATGTATTTCAAATCATCAATGCGCTGACTCACA |
| | | AATTTTCCAAACAGATTGAGAATGCAAAACCGATTAACGATGATGAAAATATTGCTAACGATTATTTGATCAAC |
| | | ATAGGAAAAGGTACAGACAAGAGTCCTACTATTATTGAAGTGCTGAATCCGGCTCATCCGCTGCATCGCCTTTA |
| | | TGGCGAACACGTGTTCCGCGCGCTGAAACGGTATCTGCCCCTGGTTACTATTTTTGAAGTTCCGTCTATCAATAC |
| | | GGAAGATATTATTAAAGATACCGATGATATACTCCCCGTTCTCTTTCAAGACTTATATGAATTCATCACCCTGCT |
| | | TTTTTACGAGCTGGTTAAGTTTAGAGACGCCTATACCCACGCACTCGCTCTGGATGAACAGGGGAATATCGTACC |
| | | CAGAAAAATTGACCTGCACCCTAACCTCAGACACTATTTTGAACACCTGTTTTACAAAGCACCAGATTATACTTT |
| | | GCGCAGATTTCGTAGCACACAGGAAAAGATTGCGCATGGGTTTCATGAAAAAGATTTTGCGCTTTTAAAAGCAC |
| | | ATAACCTGTATCAAGACCCGCATTCTAACGAGCTGACGACACAAGGTCTCTTCTTTTTCACTACCCTGTTTCTGG |
| | | AACAGAAATATGCATATCAGTTTTTGAAGCGTTTCAACGGCTTCAAAAATGAGACTATCCAGCCTTTTAAAGCC |
| | | ACCTTGCGCGTGTTCACCGCATTCACCGTCAAACTTCCAAACGACAAACTTGCGAGTGATCATCCGGGTCAGTCA |
| | | TTCTTATTGGATATGCTCAATGAATTAAACAAAATTCCTCAGTCGCTGTTCAAACACCTGACACCGAAAGATCAG |
| | | GAAATGTTTGAGCGTATCAATGACGAGGCACTGACCAACATATTAAAAAATACCGATGAGGCTTCCTTTAAGGA |
| | | TGTAGACGATCTGCTGGGCAAGATGGCTCCGCTCATACGCCGGGAAGACCGTTTCCCTTACTTTGCTCTTTGGTT |
| | | TTTAGAGCAGACTAAGGCATTCAACCATATCCGTTTCCAGATTAGTATTGGGAAAATCGCCTACTCCCCATACGA |
| | | TAAGACCATCATAGGTGAGACGAGCCCGCGTATCATCAAATTTGATATCCATGGATTTGGCTCATTGGCCGAATT |
| | | CTCTGATGAAGATACGCTGTTAACGACCCTTAAACGGCAACTTCCCGCAGGCAGCCCACATGCATCATTGACTTT |
| | | TTCTCAATACGCTCCGCACTATCATATTAAGAATAATAAAATCGGCATTTTGGTGGATTACCCGTCGGGTGATCG |
| | | TCCATTATCCAACATCACACAACAGCCAGACGGAAAAATTAAAGTCAACCATATTAAACCGACAGCCTTCATCA |
| | | GCATTAACGAGTTACCAAAGTTGGTTGTTCTTGCTCTTCTCGGCACAAACAAAGACAATAAACCTATCGGTAATG |
| | | ATATTGAGCGTTTAATACGCAGCTTTATCACGAAATTCAAAAATCAGTTATTTCATCTGGCCCCTTTAACGAAAA |
| | | TTAAAAACGGACTTACCTTTTCACCTGAGACATTCACGCGTCGAATGGATAAAGACAAAATGGTCAAAGGACGC |
| | | ATTCCAAAAAGCGAACTGGCGAAGCGGGAAGAGTACAAAATCTATCTGGAAAAACGAAAGGCTGTGCTGCAAG |
| | | GTACAACAGACTTAAAGCTGAATGTTAATCAGCTGCCCACGAAAATACAGAACTATCTCCTGAATATTGATGAG |
| | | GTATCGGATGAAAAATCCATCCACCTCAGAATAAAGACTATGATCAAAGAGGTTGAATACCGCCTTAAGAAAAT |
| | | TGATCGAAACAAGCTGCCGAAAGTAGGTGACATTGCCACCTATATAGCCCGTGACATGCTTGATATGGTGGCAG |
| | | ATGTGCGGACCAAATCTCGTATCACCTCAGCTTATTATAGTCAGATTCAACGTGCGCTGGCTTATTTTGCCCTCG |
| | | ATAAAACCCGGTTAATAGACCTGTGTGATGAGCTGGGCTTGTTCAACCATCAAACGGGGCATGTCTTTCTCATCC |
| | | GCGAAATTTTCCTGAATAATCATCACATTCTGGGTGTCTATCGTGAATACCTTACGAAAAAGCAAAATTGGATA |
| | | GACCAAAACCTGATTGTGAAAATCCAGGAAAGAGGCAAGCGCCTGACAAAATATATCCTGCCCACCAATCGGC |
| | | CATTGCCCTATAAGATGAAGCAAAGCATAAACAAAGGTTTTGAATTCAATGCGTGGCTCCAGCACAAATCGACG |
| | | ATGCCTATTGATCTGCCCACTACCTTGTTTGACGAGAGCTTAACTAGTTTATTAAAAAAAACCATCGCTCAAAAA |
| | | TTAGGAATTGCCAGTAAAAGTGAGGACAAATTCGGGAGACTGTTGGACCGGTTATTGAAACAGGACACCCAAC |
| | | CTTTTTACAAGTTTGAACGCATCTATAGAAAACCCTTCTCTCAGGAAGAGCCGGAACCTTTCAATATTTTAAAGC |
| | | TGACACACACAGCGCTCAAACAGCGTTTCGGTAAGCCGGCCGAGGTAAACGAAAAGATAATTCGTCATCATCAG |
| | | CGCAAAGATCAGATTGTACGACTGATGTGCGAGCATCTGTTTAAAAATAACTCAGAACTTGCGTTTGAAGGGGA |
| | | GTTTTTACTTGAAAATCTTCATCCGGCATCTCGCAACAATCCGTTAGATGCACCTATGGTTTTCGAGCAAACGAT |
| | | TCCACATACCCATAAAAAGATTGTTGCACAGGATAGCAATAAGTGGATACAGGAAGTGAAGAAAACAAATAAT |
| | | CGTAACAAAGCCTGGTATCGATGGACACTGAAGGATTTTGGGACTTTTCGTAAAATTATTCACGATCGCCGGTTA |
| | | CCGGCACTGGTTAATAGTATTACAGGTGATGAAATTGATTTTGACTTGGTCCAGTTTGAATTGGCGGCCTATGAT |
| | | CGTGCCCGGGAAACGATATTTGACCAGCTCTTTTTGCTGGAACAGTTTATTGCGGATAAAGACATGAGTTATATC |
| | | AAGCAGCACAAAGCGATTGAAAGAATGCAAGATGGAAAAGGTTTTGATGAGGTGCAGTTCAACACATACGCCG |
| | | ATTGGCTTTCGGATAAAGGATTGCTGACTCCTGATGAGATTAATGGTTTAAAAGAAATCCGTAATCGTTTTTCTC |
| | | ACAATCAATGGCCGGGCATTTCCAAACTTGGCATTCAGCCGATATCCCAGGAACAACAGTCTGCATTTGTCACTC |
| | | ATCTCAATATAAAGGGTTGGGTGAATGAACATCATACACCACTGGCGATGTTTATGGAAAAACTTGTTATTAATT |
| | | GGGTTTCGAAAGCCATAACTTAA |
| 293 | JW13116 | ATGGGCGTCGAAAAAAATAAAGTCTTCGAAAGCGTGATAATGAACTTTGATCAAGAGCGAAAATATGGATTCAT |
| | | TGAGTACAAAGAGACAAATAATTTGTTTTTCCACATGGAAAACGTTAAAAATCCGAAGGAAATAGTGAAGGGC |
| | | GCTAAAGTGCGTTTCGAAATATATGAGAATCCTAAGCCCAAAAAACAGAATCAGCGTTTTAGCGCTATAAACGT |
| | | AGAGGTGATAACCGACGAAACTCACAAAGAGGCGAAGATTCAGAAAAATGAGTTTAAAACATTTGACCAGTTC |
| | | ACGAAAGAATTACAGGAAACTCAGAAAGTCAATGGTGAAACCAAAAAAGAACATATCACAAAAAACAAACAC |
| | | ACCAACGTTAAAGCGGCTGGTGTAAAGTCTGTTTTTGCCGTTGATGATGGGAATGTCTTAATTACGTCCTTTGGC |
| | | CGCGGAAATGCGGCCGATATCGAAACTTTAAAAAGCGATGATGATAAAACAATCAACCTCACAGAAACCGAAA |
| | | ACCAGAAAAAGTACGTCGTGACTAATAAACGTTCGAATGTAAAAGGTTTAGCTGATAATCCGACAAAAGTTGAA |
| | | AGCATCATTCCGGGCGAGACCCAAATTGGGTTTAAAAGTATTCTGGAAAAACACTTCTTTGGTCGGACGTTCAA |
| | | CGATAATATCCATATTCAGATTATCCATAACATTCTTGACATAAAGAAGATCCTCGCCGTCCATACGAATAATAT |
| | | AGTGTATGCACTCGACAATATTCACGAGCGTGGGAGAGAAAATTCGGCCGAAAAGCCAATCGATATGATAGGT |
| | | GCAGGGGGTATTTCGACAAGTAAAGAATATGAACAATATTGCTCCGAGAAGAGCGATTACGAAGATAATTTCCT |
| | | CAAACAGTTAATAAACAATGAACGCATTGCGTACTTTGGCAACGCGTTTTTTAAAGATGAAGGGAACAAAAAAG |
| | | TAATGCGTACCGAGAAAGAAATCTATTACATTCTGGGGATGCTTAATGAAGTGCGTAATGTTTCTACACATTTTA |
| | | CAGAAGAAGATAACCGAGATTGGGCAAAGGCTAATCTGTATAATCTGAGCAACCGATTAAAAGTGGGCTCAAA |
| | | GGAAGTGCTGAACCAGTTATACAAAGAGAAAATAGACAAGATAGATGCAAACGGGTTTGTAAACAAGGGCTGC |
| | | AAACGGGACTTTAGTATCCTGTTTAAGAGTCTGAATTTGACTACCGATAAAGATAAAGGTGAACTGGTAGTCGG |
| | | ATTTTACGATTTCAGCATACGCAAAAATTATAAGAACATTGGCTTTTCAATCAAAACGCTGCGTGAGTATATGCT |
| | | GAAAATTTCAAATTCTACGCTTTGTGCAGACACAATTTCCAATAATGCAATCCGCCCCAAGGCCTATAAACTTTA |
| | | TGACTTTATCATCTGGCACTATTACATGAATAAGCCTGATAAAATCAATGACTTTGTTGAGAAACTCAGAACCCA |
| | | AAACAAAAACGATGAGAAAATTAAACTTTATTATGACGAAGCGGTCTGTTTGCTGAGTGAGTTAGGACGTGAGA |
| | | TTCATACCATGACGAGTTGTGTGCATAACATCGAAAATACGAGTTATGAAATTACCGATAAAAAGCAGAAAGAA |
| | | TACTACAAAATGCAGATAAACAGCCTGAACTCTGCCGATAAAGTATCCGATTTTTCAAAAGTGATCTATCTTGTA |
| | | ACATTATTTCTGGACGGAAAAGAGATTAACGATTTGCTGACGACACTGATTAATAAGTTCGACAACATCGCAAG |
| | | TCTGCTTTCCGTGTTAGAAAAACAAAGCGGAAAGAAAGTTGAGTTTGTTGAAAATTATTCGTTCTTCAATAGTTC |
| | | GAACTTGCTGAAGGAGAAAACTTTAAACAAATCCGAAAACTATACTTGCAAGATTGTTGAAGAATTACGTGAAA |
| | | TCAATAGTTTTGCACGCATGACAGGCGACTGTAAAATCCGGAAATCAGCTTTTGAAGATGCCAGCCAGCTGCTC |
| | | GGTTATCACGATAAAACTGTTAATAACTTGTTTGAAGTACTGCGGCTGAAAGAACTTGAGAGCAAAGACTGGAA |
| | | AAAGAGAACGGATGACGAACAACAGGAATATGATCGTCTGTTAAACAAACACCATTACTTCAAATCCGGAAAA |
| | | AAACTTCCTGATACCGGTCTGCGCAACTTCATTATTAACAACGTGATTGAATCACGCCGCTTCAATTACATTGTG |
| | | CGTTATGCGGACCCGAAAAAAATACGCAAATGCACTGAGAACAATGAGCTCTTGAAATTTGCCTTTAAAGATGT |
| | | CCCGGATTCTCAAGTTGACAGATATTACAATATTTGCGTTACCAATAAAATTACGAATGCTACCCGCGAAGAGA |
| | | AAATTGAACGTCTGGTGGACATCATTAAGAGTATGAATCTGTCCAAGGTGGCAACAGTAAAACAGCGGGATAA |
| | | GCAAGACAATGTAGAAAAGCAGAAACAACTTGCGATTATGTCGCTCTATCTGAATATCCTCTACCAGATAGCAA |
| | | AAAACCTGGTTTATGTTAATTCTCGCTATGTTATGGCGTTCCATTCACTGGAACGAGACTCTCAAATGTTGTTTG |
| | | ATGCCTATTATGATGTAAAACGCGGTTATTGCGATCTGTCAACCGTGCTGCTGTTTGGCGTCGATGATCTGCAGA |
| | | ATAGAAATCGTGGCTCCTATAAGTATTTGAGAGATAACCGGCGGAGCAATAAAGATGTGATAGAAACCTTCGGT |
| | | GACTTTAAAGGAAAAGTTTCAAAGGTTGTTGAGAAGAAAAATCAAGGCTTAACGAATGAAATCTATGACTCGTT |
| | | ATGTAATGTCGCGGGCACTACCAAGACTGAGGTCCAGAACGAAATTAAAAGCATTTTAAAAAGTAATGGTTTGG |
| | | ACGAATCGGCATCTTCTTATCTGAGCCATAAACTTGTTAACAAGGTTCATTCTTACAAATACCTTAAACAAAACC |
| | | TTGATTGTGCGGATAATACTATGATTAACCAATTTCGAAACAACGTTGCTCATCTGAATACAATTCGTAATATGG |
| | | ATGGTATTGAGAATGTCACCGGTATTACCTCTTATTTCCAGATCTACCATTATCTTATGCAGAAAGCCTTGTATA |
| | | AAGAGTTTAAAAAGTGTAGAGAAAATGCCGTGCGTAAGTGGATCCCGTACATAACTGAAAATGCCGAACCTAA |
| | | GTATGTATATTGGAACAAGAAAGAGCAGCAGGAAGTTGAGGTGTCCTTCAATCCAAAGATTTTCGGGTATATGG |
| | | AAAATATCAAAAATCATTCAAACACCTACTGCAAAGATTTTGTCAAAGCATTGTGTGCTCCATTTGCTTACAATC |
| | | TTCCCCGCTTCAAAAATCTTTCAATTGAAGAGCTGTTTGATATGCACGAGCTTTCAGAAGAACCAAAAGAGTCTA |
| | | TGAAGTTAACGGACTAA |
| 294 | JW13117 | ATGGAAAAATCCAAGTTAGATAACGAGATGACGTTTGCAGAAGATGCATACTACTTTGCACATTACGCCAATAT |
| | | GGCCCGCCACAATGCGTATTTGATTCTTAATCATATTAATAAAAGCATTTTCGGGAATGAGTCAACTCTGAAAGA |
| | | AGAAAATCTTCAAATCGCGATTATCAAAATTCTTGATCCTAAAAATGGTGCTGATCATCATGCTAAAGCGATTA |
| | | AACTGTTAAACCATCACTTCCCATTCATCGCCTGCTATTATAAAGAAGATATTAAGAAAGCCGTTGAGAATAGT |
| | | GCCTATAAAGAAATGAACGATAAGAAGTTAGACCATCTTAAAGAACCCCTCACCTTGAAACAGATTTTAAAAAC |
| | | GTTAACCGGGATCCGTAATGAATTAAACCATTACGGATATAGTCGCAAAGAACACCCCATCCCTTTTTTTCAGGA |
| | | TCTGTTTAATGACAGCATACAGCTGGCGAAAAAACGTATGATTGAATTCAATGAAACCCGGTTTAATTCCATCC |
| | | AGGATTCACCTTACCATAAAATGGTCGATCCGAAAGACTCGGTTTTAACAGATACAGGCAAACTGTTTTTCTTAT |
| | | GCCTGTTTCTGGATAAAAAAAATGCCTTTGAAATGCTGGCTAAAGTCAAGGGATTCAAGAATCGAACAGACGAG |
| | | AAATATAGAGCAACCCTCGAATGTTATACCCAACTGTGTTGCAAAACACCGTACCCGAAACTCGAAAGCTCCGA |
| | | TATTTCTCTGGATATCCTCAACGAATTAAACCGTTGCCCGAATGCACTCTACCAAGTACTGAGTGAAGAAGATA |
| | | AACTGAAATTCAAAAAAGAACCCAACGAAGAAACAGCGGAAGAGCTGAACCCGATTATGAAACGGTATGACGA |
| | | TCGATCGGCATATCTCGCACTTCGTTACTTTGATAGAACGAAATGTCTGGATAATATTACGTTTCATCTCCAATT |
| | | AGGGCGTAAAGTTATGGGCGAACCTCATAAGAAGGTTTTTGATGGTAAAGAACGTACCCATCAGATTCTCAAAG |
| | | AGATGCGCGTTCACGATAAACTTTCCACTTATGAAACAGAAGAAGCCCAAACATTCTTTCAATCAAAAATTGAA |
| | | TCAAATGAAGTAGAACTCTTTGCCCCACACTACCGCATGGTGGGTAATCGTATAGGCTTGTCTTTTGGTACGCCA |
| | | TCAGAAAAAAAGGAAGGCTATAGTATTCCTATAAACAATCAGCCGGCACCTCAGGCAATCCTTAGTACCAGTGA |
| | | ACTGGGTGCACTGTATATCTATAACCGTCTGTATAAAGAGAACAAAATTACGCAATCTCCGAAAAATCTGATGT |
| | | CTACTTATCTCCAGACAATGAATAGATTCATCAAAGATTGGAAGAACGATGGTATCAAGCCGGAGACTAGCAGC |
| | | AACAAAATCATTAAAGAACGAAATCAGGAACGCACAAAGCAGACGATTAATGAACTGAAACGTCGGACAGACC |
| | | ATCTTCAAAAACTGCTGGATGAGAAATATGCGGGATTAAAAGTCAAGCACCTTCCGGATATTTGCAGAGAACAT |
| | | CTCCTGAATTATCAATCGGACGATAATATTCAGCAGGCGATCGACTTTCTTAAAAAAACACTCTCTGACACGAAT |
| | | TTCAAAAAGAAACAATTGGAACAGTTTGAGCGTAACATGGTTTACCGCAGCTCGAATTCAAAACAGCTCAAAAT |
| | | AGGCGAGATTGCGCAGGAATTGGCCCGGGACATCGTTTTCTTTCTCAAACCAATAAAGAAAGAGATCACCAATC |
| | | GAAACAAAGAGAAAGTCACTGTTTTTCAGAAAATAAACAATCAAGACTTTGACATTTTGCAGAACCGCTTGGCT |
| | | TATTTTTCACTTTACAAAGAGGAACTGTGTTCTTTTTTTGATGATCTTTTGAAAAAGAAACACATGAAACACCCT |
| | | TTTTTGGAAAATGTAAAAGCAGAAACCAAATGGTCCAATGGCCTGATAGAATTTTATTTGAAATACCTGGAGCA |
| | | TAAACTGAAGTGGTTGAATACGTATACCAAGAATGCTCCAAATAACACCACTCGACCAAATTATACGACTTCAA |
| | | ATCGCATAAACAAAAACCGCAGCACTCCAAATAAGAAGGATGAGATAGAAGAAAAGGAGATCGATCCGGAGA |
| | | AAATAAAAGAGACATTCATCGAAAAATACAACTATTTCTTGAAGGTGAAAACCAGTTCTCCCAAAAACTATCAG |
| | | GGTAAAGCGATTTTTATTCCGGTGGGGTTATTCAACGATGCCATCACCAAAGCCATGAAAGAGAGTGGTTATCA |
| | | GGTGGAAGATGACGAGAAGACTCCCAATGCTGCTTATTGGATTAAGAGTTATTTTCATGGAGCTACCCAGCCCT |
| | | TTTATCAACTGCCTCGCTACTATAATCGGGCTAAGAAAGATGAAGAGCGCGACCTGGTAGAAGCTGCGTTACTT |
| | | AAAAAAGAGATAAAGAATTACTATGTGAAGGCAAACAAAAAGACACAGAACGACTTCAAACAGCTGGCAAAA |
| | | AGAATCAGAAAGCAAGAGGAAGAGATTCTGACTCAGCAGACCTCGGATCGCGTACTGTTTATGATGGCGACGG |
| | | ACCTGCTGAACGGTGGAAGCGACGGCCGTCAGACGATTGAGTGTGAACAGATAAACACATTAGGATTCAACTTA |
| | | GGTGAAAACAAGATTCTGGATGAGAATCGTAAAGTGTCCATGAATATCTACGGGCGGAAAGTCGTTTCCGAACT |
| | | TCCAATTAAACGTTACGGTGAATTCAGACGGTTTATGAAAGACCGTCGCCTGGAGAACCTGATTAACTATTTCCC |
| | | TGAAGGCAAGGACATTGAACTTGGGGTGCTTAAAAGTGGTGAGGTGGAAGCTGTAGGCGAGCGCTTTTCAAGCT |
| | | CAACTTTAGCCAATGAACTCGAATTTTTTGATAGCCAGCGTTGGCTGTTAATGAAGTCTATATACGAATTTGAAA |
| | | AGAAGATCTATACCCATTTCTTCGATGTTCTGACTAAAAATGTGCCGGAAAAAGAGATGATGGAAAATAACTAT |
| | | ATCGCGCATTATCACTATATAGAGTGTGCCAAGAAGCTGGAACTGTCTGTCTCTGAGTTGGAAAAAGATGATTTT |
| | | AAAGAGTTACGTAACAAGATACAGCACAATGAGATTCCGTACTCCCAGTGGATTAAGAATCGCATATCGACCAA |
| | | AGATGGCCATCTGATTACCAAACAAATCGTGGAGTTAGTTTTGAGCATCTATCAGCAAATGAATAACGAACTTG |
| | | ACCAATTAATTCGGAAATAA |
| 295 | JW13118 | ATGAACCATACGATAGGTAAAGGAATTTCTTATAATCCGTTTAAACACCAAGACAAACACTACTTTGGTAGCTT |
| | | CTTTAATCTTGCGGATAACAATATTAAAGATGTATTCAACGAAATTGCACAACGCCTGAATCATAAAGACTCAA |
| | | ACCCTAAAAACATTATTAACCATTACACTGACAAAAACATTTCGCTTGTTGATTTCGAGAAGTTTGTTAGTTTGC |
| | | TGTCCAATTATTTCCCGATTGTGAACGAAATTGACCAGATTAATTGTGACGCCAAAGGTAGTAATGGTAATTATA |
| | | AGCCTAAAACGAAAGAAGAGCGCCTGGGCTACTTCAAAAAAACTAGCTATTCATTACTTGAGGCTATTGATAAA |
| | | CTGCGCAATTACTATACCCATTATCACCATGAACCGATTGAAATTGATCTGAACCTGTTTCCATTTTTAGACCAT |
| | | GTGCTGCTCAAAACGGCTTTAAACACTAAAAAAAATTATCTCAAAAAGGGCAAGACTAAAGAGCTGATTAAAG |
| | | ATAATCTGAAGCAGCAGTTCGATTTCATCTTCAACATTAAAGTCGAGGAATATTTGAAAGAAAAAAACCCGACT |
| | | TCGAAAAAAATTGAACGTGCTGGCGGGAAAATAGAGAATTTTAACAAAAGCCTCGTTGAAGAAATAACATCTG |
| | | CAATCTACAATGATGCGATAAAAGATTTTATCTATTCCAAAGGTGAAAATGCAGAACTTTCCAACCGAAAAAAA |
| | | ACATCGTTTAGCGACAAGGATTATTTCTACTATAATGATGATGACCACTTCAGTTTACCTATTTCTTCAAGCGGG |
| | | ATTGTTTTTTTATTGTCATGTTTTCTCAATCGTAAGGAAATCGAAAGTCTGAAAGACAATATAACTGGCTATAAA |
| | | GGTAAGATTCCAAACAGTGAACTGACATTGAAACATAATGGTATTCGTTTTATGACCACTCACCGCATCTATTCT |
| | | CATTGGTGCTACAAAGGTCTGAAAACGAAAATCAGAACCTCAGAAAAGGCCACACGTGAAACTTTATTGCTCCA |
| | | GATGGTCGATGAGCTCAGCAAAGTGCCTGACGTCGTATATCAGAACATAAGTGATGACTTAAAGAAGACCTTCA |
| | | TCAAGGATCTGAACGAATATTACATAGATAATCAAGAGAACCAAGAGAATCTTTACAACTCAAAAGTGGTTCAT |
| | | CCGGTCATTCGTAAACGGTATGAAGATAAGTTCAATTACTTCGCGGTACGTTTTCTGGATGAGTTTTTAGATTTT |
| | | CCTACGTTGCGGTTTCAGGTGCATCTGGGCAATTACCTGGAACATTCTACGTCAAAAAACATTGGAAACTCGAA |
| | | AACCAATAGAGAGATAAAAAAGAAGATTTTTGTCTTTGGGAAACTCAGTGAGGTGAACAAGTTAAAAAATGAT |
| | | TTCTTTGAGAAGAATAAGCAAAATGATACCAGCTGGCAGTTTTTTCCAAATCCATCGTATCATTTCCCCATGGAA |
| | | AACAGTAATGACTTAAAAAAAGCCAATAAGATCGGAATCTACATTAACCATGATAAGTCTATTAATAAGTTCAA |
| | | ACATCAGACGCAGAAACAAACACATGAAAACAAAATCAAACTGATCCAAAAGGTGATCGGTGATAAGTCGAAA |
| | | ATCAAAATTGGTCAACCGATTGCATATCTCAGTATGAATGATATCCACTCCATCCTTTTTGAAGCCTTAAAGAAT |
| | | CCGAAAATTAAGGAATCTGGGAAAATCGACGGGGAGCACATTGAGTATAAGATAACCCATCAGATCAATAAGC |
| | | AGATTAAAGAGATACAAAACAAAAACGAGAATGCAAAAATCATAAAGAACCAGCAGTCAAAACAGGAGACAG |
| | | ACTTTAACATCGATAAGCTGATCAAAGATGTTAACAAGGAAATCACAATTAGCGAGGACAAAATCCAGACATTG |
| | | AAAGAAAATAAAAAGCAGTATCAGAATTACCAGCGCAGCAAAAAATCCGAAAAACACAACAATTCCAGAAAA |
| | | CGCAACCACATCCTCTATAATTCTGAAAAAGGAGAAATTGCGACTTGGCTTGCGAACGACATCAAACGTTTTTTT |
| | | TCAAAAGATTTTAAAGATAAGTGGAAAGGACATCACCATAGTAATTTTCAGCGAAATCTTGCTTATTACAATAC |
| | | CCAAAAAAAAGAAGTTGAATTGCTTCTGAAAGGCTTGGATTATCGCAAAACCATACCCATGCTGGACTTCTCTA |
| | | AGAATTCGTTTCTGGATTTCTATAGAAAGTATCTGGAAAAACGTAAGCAGTATTTTAAGAATTTGTTGAGTGAAC |
| | | TTAAGAATCACAAAAGCGGCAAACACGATAGTATTGAAAAGCTTAAGAAAAAATGTTTCACGCACTTCAAAAA |
| | | GCGGAATTATCGAAACAAAACCATCGAAGATAAAATACAGAACATGTTAGCCAATCCCATTTTTATTAAACGTG |
| | | GCTTTATGGATACTAAACCCACAATGATACCAGGCAAGAATTTTAATCAAAATAAAAGCGATTTTGCTGATTGG |
| | | TTCGTTGCGTTTAAAGAATTTTCGGAATACCAAAAGTTTTATGATACCAATCTTTATCCGATTGATCTGAGCCAG |
| | | AAATCAAAATCTGCGACGAAAAAGATCAACACTGAAATATACAAACAGAAAAAAAATGATTGGGCAATTTGGC |
| | | AGATGACGAAGTATATTTTCAAAGACATATTTCAGCAAGACTCCCAAAACATTTCCCTGTCTGAAATATACCAG |
| | | AGTAGACAGGAACCGATATCAAATGCCAAAAACACAGCCACGGACAAACGCAAACAGAATAGCATTTGGGACA |
| | | GCACAATTGATCTTCAGCTGAATCGGAAGATTAAAATTTCCGATGTAAAGCTGAAAGATATTGGCAACTTCCGG |
| | | AAATATGAGAATGATCAGCGTATCAACACCTTTTTAGACTATGGAGATTTTTCCGATTGGATGGCCTATCTTCCC |
| | | AATAATTGGCAGGAAAACTTTGAATATAAACCGATCAACGTCATTGATATTCAGTTAGACGATTATGAGAAAAT |
| | | ACGGCAAAATGTTTTATTGCAAGAGGTGCAAAATCTTGAACGTGAGATCTACAACAATGTATCAAATAAAAACG |
| | | AAATTTTAGTGCAGGACAGCAAAGGGAATAAAAACCCTAATTTTAAAGCGTACATCTTAAAGGGTTTACTGAAA |
| | | CAAATCAAAAATCTGGACATCACAAACTTTAAACTGCCACAGAATGCAAAAGAATTTGATACACTGACCTTCAA |
| | | ACATCTCCAGACACTGTCTGTAATAGAGCAAAAAACGACCTTGCTGATACTGCTGCGCAATAAGTTTGCACATA |
| | | ACCAGCTGCCTAATAAAGCAGCTTATGAATTTTGCCAAACTATTTTAGAGCGAGAAAAATCTCAGACCTATGCT |
| | | CATTACTATTCAGAGTTATTCAAAACCCTGAAAAAAGAGCTGACCGACATTTGA |
| 296 | JW13119 | ATGATGGAGACCATTAAACAGGAAATTGGCAAAGAATACAATCCGTTTAAAACAGAAGACAAACACTATTTCG |
| | | GTGGGTTCTTTAACCTGGCGGATAATAATATCAAAGAAGCTTTCAACGAAGTTAGTATACGACTGTCCCAAAAA |
| | | GGAGCATCGCCAAAAACCCTGATTGAAAAATATACAAAAGAAAACGTGTCCTTAGTGGACTATGAACGCTTCGT |
| | | CATTCTTCTCTCGGATTACTTCCCGATCATCAAAGAAATAGATCAAATCAATAAAGATGAGAAAGACAACAAAG |
| | | GCATTCTGATCCCTAAAACTAAGAATGACCGGATTAAAGATTTCGAGGTAATTTTGGTGTCAATGATCGATGCC |
| | | ATTGAAAATCTCAGAAACTATTACACACACTTTTATCATGAGTCAATCGAGATCGACAATAAAATGTTTTATTTT |
| | | CTTGATAAGGTCCTGTTCAAAACCGTTTTAGATACTAAGCGCAACTATCTTAAAGAGGATAAAACCAAGGAGAT |
| | | GATTAAAAACACGCTTAAGGAAGACTTCAATAAGATTTTTAACCTGAAAGTAGACGAGTATCTGACAAAAGACA |
| | | ATCTGACGTCAAAAAAAATAAAAAAGGCAAGAAAATACGGTAATAGTTTCGATATTGGTCTGACGAAAGAAAT |
| | | CACGGCATCAATCTATAACGACGCGATAAAATATTTCACATACGATAAAAACGAGAAGTCAATATTATCCGATT |
| | | CACGCAAAACGGCCTTTAACGAACGTGATTATTTTTATCGGGACAAAAATTTTGATCTCCCAATTTCTAGCAGCG |
| | | GAATTATATTTCTGCTGTCTTGTTTTTTGAACCGCAAAGAAATTGAGGACTTGAAATCTAACATCAAAGGCTATA |
| | | AAGGCAAGGTAACTAAAAACGAAGAGCCGACTTTAGAGAAAAATAGCATTCGTTTTATGACGACACATAGAAT |
| | | CTATTCTATTTGGCACTACAAAGGGCTCAAAAACAAGATTAAGACATCGGAAAGTGCGACGAAAGAAACTCTGC |
| | | TCATGCAGATGATTGATGAAGTAAGCAAAGTTCCCAATGTGGTGTATCAAAATCTTTCCAAAGATCTCAAAGAT |
| | | AGTTTTATCGAAGATTGGAACGAATATTACAAACACAACGAAGAAAATACCGAAAATACAATTAACTCGAAAG |
| | | TGATTCATTCCATTGTTCGCCGACGTTATAAAGACAAATTCAATTATTTTGCCATTCGTTTCCTGGATGAGTTTGT |
| | | TAATTTTCCTTCACTGCGTTTTCAGGTCTATCTTGGAAATTATCTGGTAGATTCCCGTCCCAAAGAAAGCGCAAA |
| | | TGTGATTACCGAGCGCGAAATAAAGAAGAAAATCTTTGTTTTTGGTAAACTTAACGAAGTGAACCAGTTAAAGA |
| | | ACGACTTTTTCAGCAAAATTGAAGAAGATAAACTGGAGACCAATTTGGAAGTTTTCCCGAATCCGAGTTATCAT |
| | | TTCCCGATGGAGAATAGTGAGGAATTGAAAAGTGCTAACAAAATCGGAATCTATCTTTCAGATATTGAGAATCT |
| | | GCTGGAACCCTTCAAAAAACACCTGAATAAGAATACTAGTCAAGAAAAGATATCGTTAATAGAGGATATCACG |
| | | CAACAGAATGAGAATATCAAAATTGGGCAGCCTTTGGCGTATCTGAGTATGAATGATATACATTCTGTCATTTTT |
| | | GAGGTGCTGAAAAACCCCATTATTAAAGATGGCAAGATTGATGGGAAAAGCGTTGAAAAGAAGATAAAAAACC |
| | | AGATCTACAAGCAAATCAATGAGATCAAGAACAAAAAGGAAGAATCCAAAATCATTAAAAACTTTCGCAAAAA |
| | | AACAGAAAGTGACCTTGACCTTAAAAAAATGATTGATGATCTGATCAAAGAAATTAAAATCACCGATGACAAG |
| | | ATTAAAAAGCTGGATGAAAACAAGAAGGAATATTTTGATTACCAGAAGAATAAAACCGCTAAAAAGCGGAATT |
| | | ACATCTTACAGAATTCTGAAAAAGGAGAAATAGCAACTTGGCTTGCCAATGATATAAAGCGCTTTTTTCCTAAA |
| | | GAATTCAAAGAAAAATGGAAAGGTTATCATCATCGTGAGTTACAGTTAAGCATTGCTTATTATGATACGCAAAA |
| | | ACAAAATGTCAAACTGCTGTTGTATGGTCTGGACTATAGAAAGGAAATCCCAATGCTGGATTTTTCTAAGCCTA |
| | | ATTTCCTGGATTTTTATGAGTATTACTTGAAAAAGCGGAAACCACAATTGTACAATCTTTTATCTGATCTTGAAA |
| | | AACTGGAAAGAGGCGAGTCCATTAACAAAGAGATGCTTCTCTCGAAATATTTTAAGCTCTTCCGTAAAAGTAAC |
| | | TACCAGAACAAATCATTGGATGATAAAATAAACACCGCGTTGGCAAATCCGATTTTTATTGAGCGTGGTTTTATG |
| | | GACGGTAATCCTACTGTTATTCCGAATACTACTATTAAAGGGAATGAAAGCCGTTTTGCCAATTGGTTCGTCGCC |
| | | TATAAACAGTTTGACGAATATCAAAAATTCTACAATACGGAAGAGTATTTCTTAGAGTTAAAGGACAAAACGCC |
| | | AAATGAAATAAAAAAGATACAGTCGCAGATCTACACACAGAAAAAAAACGATTGGGCCATCTGGAAAATGATC |
| | | AGCTACATTTTTAAAGACATTTTTAATCAGGATCTGCAGAACGTAAGCCTTTCAGAACTGTATCAGAATCGCGAG |
| | | CAGCGTTTGGAGAATGAAAAAAAATCAAAAGAAGGTGAGCGAAAACAAAACTTTTTTTGGAACAAAACCGTTG |
| | | ACCTGCAGCTTAACAATAAAATCAACATACCGGAAGTCAAGCTGAAAGATATTGGCAATTTTCGCAAATACGAA |
| | | AAGGACGAACGGATAAAGGTTTTTTTATCTTATAATGATATTACCGATTGGATGGCTTACTTACCAAATGACTGG |
| | | CAGGAAAAGCATACAATAAAGCCTATCAATGTAATTGATATACAGATTGATAAGTACGAGAAAATTCGACAGC |
| | | ATCATCTGCTCAAAGAGGTGCAAAATCTGGAAAGCGAAATCTATAATAAGGCGATTAACAAAGAAGAACTTCTC |
| | | CAGGAAGACATAAAGGGCAACAAAAATCCGAACTTCAAAAAGTATATCGTCAATGGATTACTGAAACAGATAA |
| | | AGGGCCTGAATGTTGAGAACTTTAAAATTCCGAATTCCGATAAAGAGTTTAACGCATTATCTGAAGAAACCCTT |
| | | AGATCTTACACAGAATTAGAACAAAAAGTGACCCTTTTGGTATTGATTAGAAATAAGTTTGCTCACAATCAGCT |
| | | GCCAAATAAGGATGTTTATGCGTTTACCCAAAGCATCCGGAAACGTCAGAAGAATGAAACTTATGCAGAATACT |
| | | ACTTCAACTTATTTGAGAAAATTCGCGGTGAGATATAA |
| 297 | JW13 | ATGGATACACCAAATTTCTCCGAACGTATACCTGTCAGTTTACAGAGCCACCCATATTATTTTGCACACTATCTG |
| | 120 | AATATGGCGCGGCACAATGCGTATGTGATCCTTGAATATGTGAACCGCGAACTTATTAAGCCAGGAAAAAACCT |
| | | GGACGAAGACAATCTTATTCAATCTACAGTACTCAAGGATGGCTACTTTGATCGTAAACCCGATGAGTTATCTCA |
| | | TCGTAACCGTTTATTGGTTCAGCATTTTCCGTTTCTGCGCGAAGCGGAAAATGAGGGTGCAAGAACCTGTAACCC |
| | | TGTTTCGTACAAATTAAAAACGGCTCTCGCTGCGCTTAACCAATGGCGAAATAATGCCAGCCATTACCCACTCA |
| | | ATCAAAACCATGAAAAGGATTTTGACCTTCAGCCTTTTTTTAGTTTTGCCATTGAAGCTTGTAAAAAACGAATGC |
| | | GCGAGGTGTTTCAACCCGATGATTTTTACCTGCTCGAAACTAATGAGAAACAATTTTACACCCTGCATAACGAG |
| | | AATGGTTTTACCGAAAAAGGACTTTACTGTTTCATCTGCTTTTTCCTGGAGAAGAAATATGCTTTTCAGTTTCTGG |
| | | CCGGGATCAAAGGATTCAAAAATACGACAGACAATAAGTTTCGGGCCACACTCGAGACCTTTACCGAACATTGC |
| | | TGTCGCCTGCCAAAACCAAAACTGGATAGTTCCGACATCAAGCTCGATATGCTGGGGGAGTTATCACGGTGCCC |
| | | GGCCCCATTGTTCGACTTACTGGATATAGAAGAGCGCAAGAAATTCATCCGTGAGCCGGAAGAAGTAAAACCG |
| | | GACGAATCAGGCGACCGGGAAGAAGTACAGCAGGTTTTAATGAAACGTTACGATGACCGTTTCCCCTATTTTGC |
| | | GCTCCGGTATTTCGAAGAGAAGAATCTGCTGAAAGGGATTAGCTTTCACATTCATATTGGCCGCTGGATAAAAA |
| | | GCGAACACACAAAGAAAATAATGGGCGCCGAACGCGACCGTCGTTTATTGAAAGACATCAGAACTTTTGGGGA |
| | | ACTGAAGGAATTTTCACCGGAGCATGCACCTGATTACTGGTTAAGAGATGGCATTACTCCTGATGATGTTGACC |
| | | AGTTTTCGCCCCAGTATCGCATTGTCGGGAACCGTATAGGTATAAAGCTTAATTACAATGGTCATAACCGTTGGT |
| | | CAGTGCCCGATAAAGAGATCAATGTAAAACCGGATGCAATTATCAGCACGTATGAGTTTTTAAACCTGTTCTTAT |
| | | ATGAACACCTTTATCAGAAGAAACTTACCGGCCTGTCCCCGGCAGAATTCATTCAGGATTATCTCGACCGTTTTA |
| | | ACAATTTTTTAAGTGAATTCAAAGCGGGTCATATACGCCCTGTCGGCGATTTTAGTCTTGAAAAAAGACGTGGG |
| | | CAGGGTGATGAGCCTGATCTGACTGCTCGCAGAAAAAGCCTGCAAAAAGAACTGGACCGTTTTGTTTTGAAAGG |
| | | AAAAGATCTGCCAGATAAAATTCGTGAGTATTTGCTGGGTTATAAACAAAAGAGTGAGAAAAAGCAGGCCAAA |
| | | TGGATTTTAGGCGGTATGATAAAAGAAACAGTCTATTGGCGTAACAAAGCAGAGCAGTCTCCTGAAAAGATGCG |
| | | TTCTGGCGATATGGCACAGCAGCTTGCGCGCGATATTATTTTCCTGACCCCGCCTCATACGGTTAAAGAACACAA |
| | | GCAAAAGCTGAATTCTTTGGAATACGATGTGCTGCAATATGCGCTGGCATATTTTTCTTCGAATCGTGAGAAGTT |
| | | ATATTCCTTCTTTAAAGAGCATCAATTAACTGTGAAGGGGGATCGGGCTCATCCGTTTCTGTATAAGATCCGCTT |
| | | GGATGAGTGCCAGGGAATACTGGATTTCTTTATTGTGTATATGCAGCAGAAAGAAAAATGGCTTGGTTGGCTTG |
| | | ACCGGAACCTGAAATCTCCGCGATTAAACGAAGAGGAATTCTTCAATACGTACTCCTATTTTATCAAAACTGAT |
| | | ACCAAACGGGCAATTGAGATGGATTATGAAAGCTGTCCGAATTATTTGCCACGGGGCATTTTTAATGAACCCAT |
| | | TGCTAAAGCCCTGCAAAAAGCCGGTGTGAAAATAAAAGATGAGGATAATGCATCGTATGCGCTTTCTGTATACA |
| | | GCAATGGCAAGACGCAGCCTTTCTATAACAAAGAGCGGTATTACAATAAAGGAATTTTCCGTATGGAAGAGTTG |
| | | CCGGAAAAATTGCAACCGAAAGAATTACTTGGCAAAATTCAGTGGACGATTAAAAGTTCCGGTAAAGATACGG |
| | | AAGAATTTCGCTCACTTCAGAATCTTAAAAACAGAATATTGAATACTGAAAAAGAGATCCGGTATGTGCAGAGT |
| | | ACCGATCGCGCCCTGTGGATCATGGTCGCTGATCTCTTCCCTGAAACATTCGAATTACGCCCGGACGATCTGGAA |
| | | TGCATCGGTCACGACCTGTCAGACGATCTCTTGTCGCGCCCGTACCAAATGAAAGAAAAAGTTTACAACTATAC |
| | | CATCACCGACTATCTGCCCATCAAAAGATATGGCGAATTTCGTAGATTTTTGAAAGACCGAAGACTGGAGAATC |
| | | TCCTGACGTATTTTGAGGAAGGTGTTCCATTGCATCGAGAAGCACTGGTTGCCGAGTTGGAAGCTTACGACCTGC |
| | | AACGCAAAAACCTGCTGGAAATAATCTATCGCTTCGAAAAACTGGTTTTCGATAGACACCGGCATGAGTTAACA |
| | | TTTTCAGGGGAAGGAGAAAATCAGTACGTCAACCACTGGGATTACCTGGACTTTGTTGCCCGAAAGTATGGATT |
| | | GAGCGCAGAGGTAAAGGAACTGAATTCAGAACGGTTTACTGAGTTGCGAAACAAGATGTTGCATAATCAGATTC |
| | | CGTATCAGTTATGGATTAAGGAAGCGATTGCTGCGCGTGAAGAGAACACCGTGTGTGGTCGTATTATGGGAATG |
| | | ATCGGTGAAATCTACGAACGCATGACAACAGAGATTGAAAAACAGATGCAGGTATAA |
| 298 | JW13121 | ATGTTTGATAACGAACAAAAGAATCTGGAAAAAGAACCTTACTGGGGAGTATTTCTGAATCAGGCACGTTTAAA |
| | | CGCCTATATTGCACTGCGCGATATTTCGGAACGATTGGAAGAAAACGCTGCCGATGAGGATAGCCTTAGTGAAT |
| | | GGCCGGTCTTGAAGTATCTTGACAATGATACAGATGCGGTCAAATCCCGCCGCATTTTTGATCTGGTGGAAAAG |
| | | CATTTCTCAATGCTCAAAATTATCTATGGCGGTGAGAAAGAAGGCGACCTTGTAAAACGTTCGAAAGAATATAA |
| | | GATAATATTAAAGTGCCTGTTTAGAGCGTTGAACTTTTATCGTAACAAATTTTGCCACATGTATTCAGGCAATAG |
| | | AGCTCGGAAGTATAATGAGAAAGAACTCATTAAGTATTTGGAAGATTGTTTTGACGCATCTGTGCGCAAAATCA |
| | | AAGAATTGCGTCGTTTGGATGAAAAAGATGTTTTACATCTTCGAAGAAAAATCGCGGAAGGCAAAGACGCGAA |
| | | CAAACGCGTTATCGATAATCCACAATTTCGTTATCCTTTCAAAAATGAGAAAGGAGAACTCAACGAAAAAGGGC |
| | | TTTATTTCCTTGCGTCTATTTTCCTGGACAAAAAAGAAGCCCATGAATTCTTAAAAAAGCAGGAATACTTTAAAA |
| | | ACGACAGCGAGCCTAAGTATCGGGCAACGTTAGAAAGTTTTTACCACTATAGAATAAAACTCCCACGTCCGGTT |
| | | ATCGAAAGTGACGTGGATAAAAATGGTCTTGCGTTAGACATGCTGAACGAGCTTAAAAAGTGTCCAAAAGAACT |
| | | GTTTGACCTGCTCTCTAAAGAACAGCAGGAAAAATTCCGCGTAGTGGATTCCGAAGACGCTGACGAAGAGGGC |
| | | AATGAAATCCTGATGCGCAGATATTCAGATCGTTTTCCTTATCTTGCGCTGCGTTATTGTGATGAGAATCAGGTG |
| | | TTCGAGAGAATTCGGTTTCAGATAGATCTCGGCCGATATTATTTCAAATTTTACCCAAAAGAAACTATAGATGGT |
| | | AAGACTCAACAACGTTCTCTGGACAAACGTCTGAAAATCTTCGGTCGTATCAAAGACGTGAAAAGCAAAGTCGA |
| | | GCAAGAATGGAGTGGTATAATAAAATCGCCCGATACCATTGAGGAGAATCCAAACGAGCCCTATAAGCTGAAA |
| | | ACCACGCCGCGTTATAACATTGTGGATAATCAGATCGGTTTTGTCATCACAGGCGACAAAAATCTTCCGGATGTC |
| | | AAACGACCCGATGGGAGAATTGAATTAGAAAAACCGGACGGTTGGCTGAGCATCTATGAATTACCTGGCATGTT |
| | | GTTTCACGGATTAAAATACGGTTTTGATAAAACAGAGAGAATGATAAAGATATACATCGAAAAACAACGGAAG |
| | | ATTTGTAAGGAAATTTGTGAAAAGGGGACAATTACACCGGATGATGGCGAATCCATGCCTGAAGCACTGAAAG |
| | | GTGGGGCAAAGGCTGCTAAACGCAACTACAGCGAGAAAAAACTCGAGCGGATGTTACAAGATACTGAACAGCG |
| | | AATCCGAGCCATTCAAACGACACAGAAACGGATGGACGAGCCAGGCAATAAGCCGGGTAAAAAAAAGTTTTTC |
| | | GATATCCGCGCAGGAAAATTAGCGGATTTTCTGGCACGCGACATTATGGCGCTGCAGAGATTCGACCCGGCCAA |
| | | ACACGGTAAAGATAAGCTCACCGCCATTAATTTTCAGGTCCTGCAGGCAACCCTTGCCTTTTACGGGGCAAAAA |
| | | AAGACGTTATCGAGGATATGTTTAAAGGTATTGGGCTGCTGGAAGGAGATAATCCCCATCCGTTTTTGAATCAG |
| | | ATTGATCCGGCTCAATATAATTCAATTGCAGGATTTTATCAGGCTTATTTGCAAAAGAAACGCTCATACCTGGAA |
| | | GATTACAGAAAAGAGGAAGAATACGACGAGCAGTTTTTACGTCCCAAACGTCAGCGTTATGCGCAAGAAAAAC |
| | | GCGAGATCAAAACCGTTGCACGTCAGCTGCTCGATAATCCTGTCAATGTACCCAAGAATTTTTTCAAAAAAGAG |
| | | ATTGAAGAGTTCGTTTTTAGCCAGGATCCGTCTCTGAAAAAATCCAAAATGAATACAGCCTATATGATCCAGGC |
| | | CTTGTTTGAAAAACATTACGGCCGCCAGCAGCCGTTCTACAGTTATAATCGCACCTATCCGGTGGTTTCAAAGGC |
| | | GATAGAGTACGGCAAAAAAGGGAAAAACAAAAAGATCGCCAAAGTACTTATGGCCATAGAGCCTAAACTGAAT |
| | | TATATGGAAATCAAAAAAATTGTAAACGAGATGCCAGACGGCCAATATGAGCCGGAAAACCTGAAAAGAAACC |
| | | TGTACGAGGGATACAAAGATTATGAGAAAGATGAGCGGATAATACGCCGTTGCAAAGTACAGGACGTGGTTAG |
| | | TTTTATGATGGTTGAAGAAACATTAAAAGATCAGCTTGATTTTAACGGAAACGTTCTGACGCTGGAAAAAATAA |
| | | CCCCGTGGGAAGCCAGCCCATTCAAAAAGCCTGTTTTATGCCATACGATTATCTCCATTCCATTCAACACCAAAG |
| | | GGGGTCATACTGACAAGGATTATGTTGATTTTATCAAGAATAATTTTGAGGGAAGCTACGATTGCGAACCAAAT |
| | | AAAATCATTCTGAAGTACAAAGTGACGTCAAAAGATACGAAATTAAAAGATATCGGCAAATATCGGATGTATTC |
| | | GCACGACCGGCGGCTGCCCGGTCTGTTAATATGGAAATATCGCCCTAACGATCAGAATGGAAACGAAATTAAGT |
| | | TCACTGAAATTGAACAGGAGATTAAGGCTTTTGAACGGCGCCGAATTGAAATTGCTCAATGTTTGTATACGCTTG |
| | | AGAAGAAGGTGATTGATTCCTGGTTCACTCAAGACGAGCTTGGTGAAGAACATATTCCCTTCAATAAGGTAATA |
| | | GACGTGATTAAGGCTAAAATGCCGAATTTCGAGGATAAGTGCAACGTTCTGTTGAAAATCCGTAACGCCATTAA |
| | | TCATAATCAGTTTCCGGTCTATGAACAGGCGATTCAAACCGCACCAGGTAAGGAAATTGCGGGTAAGATGCTGC |
| | | GTATAACCGAAAGTTACATTGAACAGATTATGGCAAAAATCGATCCTGATTTTGGGCGTACTGAAGATGCTGAG |
| | | TCTTCTCGCTAA |
| 299 | JW13122 | ATGCCAACGACGAACAATCAGAAAAAGAAAGCAGATTCTATCAAACGAACGAGCCCTTCCCCGTCGCGCATTA |
| | | ATGGGGTAGCGAAACCGCGCCCCCTGGTAGTTGATGAAAATAACAAACATGTGCTCGGGGGTTATTTTGGCCTC |
| | | GGCTTAAACAATTTCTACAAGGCTACCCTTTCGATATTCGCGCGCACCGGTATCAAGATAGCCTCAAAAAATGG |
| | | TACAGTTCTTTATCCAGAAGAAAAAATAGGGCAAGTTATACGCGTGTTACGCCGTAGCATTTCTGACACTTCCGG |
| | | CAATCTTACCCACGAAGAAAAAGTGTGGTCAAATGCCTTTAAGCTTTCATCAAGCCAGGAGATGCGCCTGCAGA |
| | | AGCTGATTTTCCATCACTTTCCAATTCTGGGTCCAATCATGGCGGACGAAGGTGCTTATAAAGCCTCTACTAATA |
| | | AAAAAAGTAAGGTTACCGACGCGTATAGTATGACCTATGGTGTAACACTTGAAGAGTGCCTGGATGTTTTAGGC |
| | | GACCTTGCACAGACGTTGGTCGATTGTCGGAATAGCTACACTCATTTTTACCCGTATAACAGTAAAGAGAGCAC |
| | | AGCAGACCAGTTGGCATTACAGTCCAAAACCGCTCGTTACCTGGATAAAGTCTTTACCGCTTCCCGTCGGCTCGA |
| | | CAAAGACCGTAACAATATCGACACGGCTATGATGGAATTCATCACCGGCTCAGCTAGAAACCTGCGTCAATATG |
| | | TTGAACGATATGGTTTTTATCCTAAGTATGACGAAACGGTTACCTACGAAAAGGATACTCGGGGCCGTGAACGG |
| | | ATGAAAAAAAAGATGACAGAACGTGATGATTTCTTTTACAAAATCGGTAACGAGAAGGAGTGTGACGGTAAGC |
| | | GCTACACGGTATTAAGCGGATTCGGCTTGAGTTTTTTTTGCGCCGTGTTCCTGTCAAAAACACAGACGCGTAATA |
| | | TGTTGGCTGATATTCGCTTATTCGAAACATCACCGTATCCCAAAGAATTAGACGATATCATTCGCGATATGATGA |
| | | GTATCTATAGAATTCGCGCTCCACGCGGTAAAAAAAAGCTTGAAGCGGGGAATACTGGTGTAACTTTGGCCTTA |
| | | GATATTCTGAACGAACTGCGCCGCTGCCCACGCGAACTGTATGATGTGTTAACAACTGAGGGGCGCAAGTTTTT |
| | | TGAAGATGAAACCGATCGAACTAAAGATGCGTTTGTTCCGGATGATGCACTGTCCCCGTATACCAGCGCCGACA |
| | | ATGCAAGCGAGACCGTTAAACGTATTCGTTGGACCGACCGATTTCCACAACTGGTGATGAAATATGTCGATGAG |
| | | ACAGAATTCTTCAAAAATATCCGTTTCCAGGTCCGTCTGGGGAAGTATCGGTTTAAGTTTTACGACAAACTTTGT |
| | | ATTAATGGCGAAGAAGATCTGCGAGGGCTGCAGAAAGAAATTAACGGATTTGGGCGCATTCAGGAGATGGAAA |
| | | GTGCCCGTAAAGAAAAATATGGCGACATGATGCAGCCAGTTGAAAGCGAAAGAATTCGCATTGATGATGACCTT |
| | | GAACTCGATCTTCTGCAATTTGCGGAAGATTCTCGTGATTCTTCACCGTATATTACAGATTCTCGAACCGCGTAT |
| | | AATATAAACAATAACCGGATAGGCCTGTATTGGAATGAATTGACGGTGAACGGACGGACAATCCCGAAACCGG |
| | | GTGACCATCTGCCCGACCTTGGCCTGACGCCTGAAGGGAAAGCGGATGTCGATATGCCGGCCCCTATGGCTATG |
| | | CTCAGTTTACATGAACTGCCCGCCATGGTCTTCTATCAGTATCTGTTATCGCAGAATGCAGACCCCAATGAGCTG |
| | | AAGACTCGTTCCGCTGAAAACCTGATTATTGGCAAGTTTAATTCCTTGCGCCGTTTATTCACGGATATTAGTGAA |
| | | GGGCGATTAGCACCCTTACCAGAGGATGGATTAGTGCGAGTTTTAGACGATCGTTATGGCCTCACTCTGAGAGA |
| | | AATTCCGGATAAACTCACAGACTTTCTCACCGGTAAAAAAGGTGCAGATCAATCTAAGCGTAGACGTGTGGTGG |
| | | CTGAACGTACACTGCTGGAATCTCTGCGTAAAGCAATCCGTCGTAGAGATCGTTATGAGGAAGACAGAAAGAA |
| | | GATTGGAGAAAAAGATAATAAGTATGCCAAAAAAGCTTACTCCGATGTACGCCATGGTTCATTAGGACGATACC |
| | | TTTCAGAATCCCTCATCGAGTGGCAACCTACTTTGGAAGGTGGAAAAGACAAACTGACGGGTATGAATTTTAAT |
| | | AAACTTCAGGCAGAGCTGGCGACCTTTAATAGTCCTGAAAAATATACCCGGCTGCGAGAGACTTTATCGGCAGC |
| | | GGGGATGTTGTCAGGCCCAATTGCACATCCTTTCTTACGCAACGTTACTAATCGTAAGATCCGGAACATTGAAG |
| | | AGTTGTATCTTATCTATCTTGATCAGGAGATAGGATATCTTAAGACCCTGCTCAACCTCGGATACAAAGGCGGTA |
| | | GACGTGATGATCGCAACATTGATTTTGAGGCATCGACCCTGAAAACTCATGTTGATGTGCGTAAACTTCCCTTAG |
| | | TGAAGGGAGAGAAACGCTGGGAAAAACGCACGCCTGATTATTGTCGTGAATTAGCGAAACGTTATCTTGAGGTT |
| | | GACGGAAGACGTACCTCTATTTGGTTGCCGGAAGGTATTTTTAGTAAAGCAATAACAGACCTTCTGACGCGCCA |
| | | CTACTCTGGTAACGATGGCCTCATGGACCGCATGCGAGATGACTCACTCAACAATAACGTCGCCTATCTGATTA |
| | | ACGCATTTTTCGAAACTGTCTTGGATGATCACTCGCAACCATTCTACATGTCCTACGATATAGATAAAAACAGCG |
| | | GAAAAGAAACACCGAATCGTTTCGCAAGAGTGTATGATCTTTTTGATATATTGAACAATCGGAAGATCAGAAAT |
| | | GCGCTGGTGAAAGTCCCTATGACCTGTAATCAGATCGCGGAGCGGTTTACTGCAAAAAATCGTGACGGTCGCAA |
| | | ACGAATCTTAGCTGAGATTGATGATCGTACCGCGGGAATGACCCCTAGAGATATCGGTTCACATGCCAGTCTGA |
| | | AAGAGGCCAAAGAGGCTATGCGTCGCAGACTGACCCACATGATCGGGGATGTGAAAGATAATGAACGTTGTGT |
| | | ACGGCGCTATAAGACACAGGACATGATTTTATTCCTGATGGCGAGCCAACTGTTAGGGGATGCTATACGCATGG |
| | | AAAAAATTGACAGTTTAGACAAGTTTCGCCTGAGTGCTGTCTGCAATGCAGGCTTTCTGAACCAAACGATGGAT |
| | | TTTGAGTTTCCGGTTCAGATGGATGGCAATGTAATCTACATCAGACAGGACAATATGAGCTTGAAAAACTACGG |
| | | AGAGTTTTACCAATTATTAAGCGACGATCGTCTGCCGTCACTGTTAGAGAAACTGGCCGATGGCGGCTCTGATC |
| | | GGATAGATTATGGTAACCTTATGGGTGAACTGACCAGCTATGACCTGCATCGGAGCCGTATATTCAAAGCCGTT |
| | | CACATGCTGGAGCGGCGCGTAACGAGTGACCGGCAGTTCAGAATATTTCTTAACAATCCGTGCGATCGTAGATT |
| | | TTACATCAATGACGATGCGTCTCGGATGGCGAAACGCAATAATTTTCGGTCTCTGTTGATGTTGTTAGAGCAGAG |
| | | CGATACTGCCTCTCTGACGGAGTCGGAACGTAACTTGATTATCTCCATACGTAATGCATTTTCGCACAACCATTA |
| | | CTCGATTGATCTGCCGCGTATTGCCCCCGAGAAACGGTTAAGAAAAACTACATTACTGAATAAAGATACACGTC |
| | | CTCTGGGCGAAAAAAAGGCCTCTTTGACAACAATTGCCACACTGATTGCTGCGCGGCTCGAAGAGCTCCAAAAA |
| | | AAATTGGAATCAAAACATTAA |
| 300 | JW13123 | ATGAATTTCGAGGAGAACGGAAATAAGTATCTGTTTGGGGCATTCCTGAATACTGCGTATGATAATTACAACGA |
| | | AGTGATAAGTCATATCTATGATGTCATTCGGAGAATCACAAGTAACAACCCGACCGAACAGATAAGCTCTTCAC |
| | | AGGCGCTGGTATCCGCAGAAGAGCAAACCTCTTCCGACGAGAAGGATAAAAACAAAGAACGTATGTCTTTTGTG |
| | | TGCACGTTTTTGATAACAGGCAATGCCACTTCACGGAAAATAGAGTCTATGCTGAAACGGGCCTTCCCTTTCTGG |
| | | GACGATTTGATTGAGATTCAGACTTCCATGTCTATGCAGCTGGATAAAACCGAAAGCATCTTTCAAGTTCAAAA |
| | | AAATTGTGTGGAAGAACTGTTCACAGCGCTCGAAAGTTGGAGAAACTATACGACTCACTACATTCACCCGGAAG |
| | | CAATCGCCTCTCCAACAATTAACCTCTTGCTGAAAAGTTGTTTTAATCAGGGTATAAAGGAGATAAAGAAACGT |
| | | TTTAACGCTCAGGATGAACAACTCAAGCATCTGTATGAAGCGGAGTTTTCTTATAATAGAGAAACAAATAAAGA |
| | | CGAGGTGAAATGTTACCATCCCGCCAACTACTATAAGTTTACTGACGAAAGCGATCTGTTGACTGATAAAGGCT |
| | | TAGCTTTCTTCACCTGTCTGTTTCTTTATAAACGTGAATCTTACCTTTTGCTTAACCAATTAAAAGGCTTTAAACG |
| | | GGCTGATGAAGAGCGTTACCGTATGACACTCGAGGCCTTTACCGTAATGCGGTTTCATGCGCCGAAACAGCTGA |
| | | TGAAACTGCAGCAACAACAAATTGATAAAGTTTCGCTGGGAATTAGTATGATCAATGAATTGGTGAAATGTCCG |
| | | AAAGAACTGTACGACTGCATTGAGGACGAGCAGAAGAAACAGTTTGAAGTTTATCCGAATGCCGAGACCGTAC |
| | | CGGATGAACAGGAAGCTATAGGAGAACTGATCCGTTATAAAGATCGTTACGAAATTCTTATGCTCAAAGCACTG |
| | | GAACACTCATCAGAGTGTAAAAACATGGGGTTCTATCTTAGTTTAGGTAAGTGCTACATGAAATGCTATGAGAA |
| | | GCATTACATTGATGGGACCACCGACAACCGCTATATCGTGCATCCGTTGTATGGGTTTGGCCGCCTGCAAAATGC |
| | | TTCGACGGATCATAAAAACCATCTGTCCTCATTCGGTGAAGAACTTCAGACGCATAGCCAGTTGCCTGTATGCCA |
| | | CTTCGCAACCGAGGACGATGCAGCTGATAGCCCTACAAGTGTTCCCTATATTAGCGAATCCTATCCGTCTTATGT |
| | | TCTCAATAATAATAATATCGGCATTAAGTGGCTTAATTCTTCGGAAAAGACAGAATATCCCAATTTTAACCTCAA |
| | | TGAAGGTAAAGTCTCGTGCCCTACGCCTGATTATTGGTTGAGCAAATATGAGCTGCCGGCGATGGCCTTTTATGC |
| | | GTACTTAAAAGAAAAACATAAAAATAAGCTGCAAGAGTATCCCTCGATTAAAGAACTGATTGATCAGTCACAGC |
| | | CGGAAAACACAAAAACATCCTCAAAAACGATTAAGAGTCGCCTTGAAGAACGTCTGAAAACATACAAGGTTGA |
| | | TGCGGCAAAAAAACTGAAACGATTAGCAGAGAAACCGGACGGCGTACAGAGCGGAAGATTAGCAGATGAACT |
| | | GGCGCGTGACATCTTATGGTTACAACCATCTTCACCTAAAGAAGGCGGAAAAGATAAACTGACGGGTGCCAACT |
| | | TTCAGGCACTGCAATACGCTCTGGCCCGCTATGGCTTTATGCGTACAACTCTTTCCCGCATTTTTACCACGGCCG |
| | | GTTTGATTGGCAGCGGTAATGCACACCCGTTTCTTGATAAAATTAATCCTCAGGATTATTCCAAACTGACGGCCT |
| | | ACTATGTTGGGTATCTCAAACAGAAGATAGTCTACATCGATAAACAGTTAGAAAAATTAGCAAGAGGTAAAGTC |
| | | AACATTCAGTTTCATCCCACGCGCAAACTGATTAAGCAAAGCCAAATCAACACCATTGAACCAGAAAAAGAGA |
| | | CCGTTTTTCTTCCCCGTAATATTTTTACAGAGAAAATAGAACAAGCCTTATGTCAAATTCAGGGTAACTTTAAGT |
| | | CGAATATCGAACGCGCTAAGACATCAGGTAAAGGAGTAAACGCCAGCAAATTGATTCACTTCTACCTGGAGTTA |
| | | TATCTCAAAGATGCAGTTACCGAATTCTACAATATTAAACGAAAATATTCCTGCTTATGTTATCCACTCATTCCA |
| | | TCAGAAAAGTATAACCTGAAAGAATATCAGAATACGGAAGAACGAAGTGAGCGCATTGACCGGATAAAAGATT |
| | | TTTACAATTTTAAAGAGGTGACCAAAAAAGCTTGTAAGGGCCAGGAGTTATCTGAAGACGAGAAAAAGCTTTAT |
| | | AAGAAATATATTGAGCTGCAGAAAGTTATCGACAATGAAAAGATGATACGCTTGCGCAAAGTCCAGGATATCA |
| | | CTCTCTATCTGTGGATGCGTCTGTTCATTGAAGAAAATCCAGAGCGCATAGTCTTCAATAAAAATGATCGGCCGG |
| | | AAAATAGTTTGCTTAAGCTGAAAGAGATGGATAACACTATCCTGGATCATACCACTGCGAGAAAAATCAATATT |
| | | GACACCCCAAACGGTAACGTGACTATTGAAGATGAATTCAAAATTAAAGACTATGGACGTATAACTACCCTGAC |
| | | CACGGAAAAGCTGCCTCAGAGCTTGATCAAATTCATTGGGATTCTGAATAAGCCTTTAGACAAAAAAGACCGCC |
| | | CATCGCTTTCATATAGCTATATCTATCATGAGACCAATGCCTTTTATCGATACCGACCAGAAATAATTAAAATGG |
| | | CTCAGGATATCGAGAATAAGATCATAAACGAGTTAAAAATCGATATTCAGCCGGAAGCGGAAGGTTACTATAA |
| | | CTTCCGTCAAATATTAGAAGAAATAAAAGAGAAGTCGAGTGACATCAATCTTGAGTTATTAATCAAAATCCGCA |
| | | ACGCGTTTTGCCACAATGAATATAAGGAATTGGACAATGATCAGCATAAGTATCTGAGTTCATTGTTTAAGCCG |
| | | ATCGCGGATATTAAGCAGACAGCTACTTTGGCATACTTATTGCACATGCTTTTTGAAACGGAATACCAGAAAGT |
| | | GATGATTCTGAAATAA |
| 301 | JW13124 | ATGAAAAAGAAAAAAGATTCTCACAAAGCAGATTGGCGCGATGATCTCATTTCAATTAAACATCTTCTGGTAAA |
| | | GCAGGAGAAAGCAAAAGCGGCATCCGCCCCGAAAAAACCCCCAGCCCCTGTTGCATTGATCGACCTCTCCCACA |
| | | TGGAAGAGCAGCGCCGCGAATTGCAGGCAGAAGATTCATCCTCTGCCAAACCTACAAAGAGTAGCGCAAAAGC |
| | | GGCGGGCATGAAATCCGTATTTTTATTAAGTGATAAAGAACTGCTCATGACCTCATTCGGGCGTGGGAACGATG |
| | | CCGTTCGGGAAATACGGGTGCAAGGCGACGAAGTACAAGCCTTAAGAGACGATCCGGCTTACCGCATCGAAAA |
| | | AGCGGATAGTGGATTTGACATCCATGGTCGTACTCCTACGAAAGGTTATACGGACGATCCACGTTACGCAGCGG |
| | | AAGGTCCACCGGAACACCGGGATCTGATCGATTGCCGAAAGGCCTTGGAAAAAATGTATTTTGGCGAGGAGTGT |
| | | GAAGATAACATACATGTACAGTTAGCTTACAGTATTTTGGATATCGAAAAGATACTGGCCGTCCATGTGAATAA |
| | | TATCGTCTATGAATTGAATAACTTGTTACGTGGTAAGGAAAAGTATTACAAAGATTGGATTGGTTATATGGGAA |
| | | AATATTACACCTATCAAGAGGTGCTTAGAAAAGAGTTCAAATCAAAGAATGATGAAAAGAATCTTACGTTTGTA |
| | | CACAACATGATTTTAAACGTCATGCAGGATAAACGTCGGATGTATTTCGGCAAAATCTTATGTGGTCCATCTATT |
| | | GCGAAAGAGGACAATCAAGAAAAAAAGAAGCAGCGTTATGAAGAAGAGCGTGAACATGCGTATTATATCTTCC |
| | | TGATTCTCGGTATGGCGAGACAAATGCTTGCGCACGGTGATGAGAGCACACGCTCATTGTTATTCTGTTTTGATG |
| | | AAGAACATGATTGCAATAAAACAAAAGAAGTTCGCATTAAGCTTCGTCAAACCTTAGATGCTGTGTATTCTGAA |
| | | CGTGTAAAAGAACTGAACGATGAGTTCATTGATACCTCTTCAAAAGACTTAGTTATGCTGTGCCGTGTAATGCAT |
| | | GCTCGAAGCCCGGAGAAAAAGAAGGCTTGTGTTCGGGATTACTATGATTTTGTTGTTCGAAAAGAGTACAAGAA |
| | | TCAGGGGTTCTCGGTAAAACGCCTGCGTGAAACTATCATTCTGCGTACTCCTCAGGCAGCCGTGGTTAATGATGC |
| | | ACAGTACGATACAGTCCGCCAAAAACTTAATAGACTGTTTGATTTCGTGATTTTTGACCATTATCGTAAGCATGC |
| | | AGCTGAAGCCGAACAGATTGTCGCCAGCCTTAGAGCAGCACAGAGCGATATTGACAAAGAGTTAATCTACCGA |
| | | CATGCAGCAAATAATCTTTGGCAGGCGATAGGGAAAACCGTGCTGCAGGACATTCTTCCTATGATGAACGGTAA |
| | | TGCAATAAAAGGCATTGCTGCAGATCCGGATATAAAGCTGTCAATGATTTCCGACATTATGATATCTGATGATG |
| | | CTGATTATTTCGTCAAATTCATATATCTCTTGACTCGGTTTCAAAATGGAAAAGAGATTAATGACCTGCTGACGA |
| | | CCTGTATCAACAAGTTTGAAAACATCACAGCTTTTATTTCCGTGATGGAAGAACAGGGTATTTTCAAAGAATTTC |
| | | GTCAGGAATATTCGATGTTTGACAACAGCGCAGCTATTGCCAAACAACTGCGCGAGGTCAACAGTTTTGCACGT |
| | | ATGTCCATGAAAAGCCAGGACTTGGGATTGGCAGACAAACTGGTCATGTTCGAAGACGCGGCCGATATTCTTGG |
| | | TTATTCAGATCCCCAGGCCGGCAAACAACTGCTGGATACGATACTGAATCCGTCGCAGAATACAGGCTTTCGAA |
| | | ACTTTATAGCGTCTAATGTCGTTAAGTCAAACCGGTTTCATTATTTGGCCTGTTTTTGCAATCCGAAAACGGTGC |
| | | GCAAGCTCGCCGAAAACAGAGCCGTGATAGACTTTGTGCTCAGCGAGATCCCGGATGCGCAGATCCTGCGTTAC |
| | | TACACTGCTTGTACCGGAGCGAAAGAAGGCGCAGATCCAGGGGAGATGCGCGCTTTTCTGGCGGAAAAACTGA |
| | | CTGGAATCACCTTTGAAGAGTTCCGTAACGTTAAACAGAAAGATCGGTTTGCTTCGCCGGAAGAGAAACTGGAC |
| | | AAAGTCCGCAAGCAAGCCATCATTCGTCTGTATCTTACCGCGCTGTATCTGCTGGTTAAAAATATGGTTTATGTT |
| | | AATAGTCGGTATTTTTTAGCTTTCCAATGCCTCGCTCGGGACAACGCGCTGCACGGTTACACCGATAATGACATA |
| | | AAGAATGATCCAGCTGCCTTTGCCCGCCGATTTTTAAGTGAGCCGCGGCATCGGAAGAATAGACGTGCTGTAGC |
| | | CAATATCGAACAGAACATGGCGAATAGTGATCCCTGGATAGTGCGGGAATACAGAAACGCCGCCATGCATTTG |
| | | AGCGCGATTCGCCGCATGCCGGAGTATATCGCCGATGTGAAACGTATTAGCTCGTATTTTGAGCTGTATCACTAT |
| | | ATTATGCAGCGTCACCTCGCGGACCAGCATACGCGAAAAGAAGGAGAGGGTGAGGAAGCGGTAAAAAATGAA |
| | | GTTGTGCTGCGCTATTTTGCTGACGTTCGCAAATATGGCTCTTACTGCAAAGACTTCCTCTGGGCGCTGAATGTT |
| | | CCTTTCGCATACAATCTTCCACGCTACAAAAACTTAAGTATTGAAAACTTATTTGACCGTAATAGACCCGGAACA |
| | | AAAGCAGGCGGGCCTACAAAATCTGCTGGCCCCACTGGGGCATAA |
| 302 | JW13125 | ATGAAAATTATACGTTCGAAGGGACAAAGTTTTACTGAGCATTCAAAAGACGAGTCTAACACTGACCATCTGGA |
| | | ACGAGGGTTAGAAGCAAAACGGGAGCGGAAATTAGTACATAGTGAACATAAAGATAAGACGTTATTTCATGGG |
| | | CGCGAATTGGCCCAGTATGTTGGAGAGGATCATGCATTAGTCCTGCGGCAGGCCATATCCGTACTGGATAAAGT |
| | | TATTCGTCCGCCGCGTCGCCTGCGTGATTTCGAGCTTCAGGCCTATGTTGACCGGGCTAAACTGGCGCATGAGTT |
| | | GTGGCTTCTGGTACTGAATAAGTGTAAAGGCGACTTCACCAAGCATCCTGATTCCGATGAGGTGCAAAAAATAT |
| | | TTGAAAAAACTTGGAGTGAACGCGCACATCCCTATACCCGTGACCGGGTTCAACCTTTAAACCCATGCGGCGAT |
| | | GAAAACGGAAAATATCTGCAGAGTCATCCCAAAGAGGGCGTGACTCGCCAGGATTTTGAAGGCCGTTGGTATTC |
| | | CGCCCTGTGGCAGGAAAATAATACGACCAGAAAGCCGGATTACGCAACGACCGCGCAAGCCATTATAGATCAT |
| | | CTTTTTAAACAGGAACTGCAGTTTGATGGGCAAGAAAGAACAAAATCAGGGCATAACGACGATAAGAATAAAA |
| | | ACAGCGGTACGCCGGAACCCACCGGAAAGGGTCTTGTAGAAGCACGCGGTCTGACTATAAAGGACAGCGTGAA |
| | | TGATCCCAGAATGGGTAAACAGGTGACAAAAAATGCTGATCTGCGTGAAGCGTCGAAAGAAATAGACAGCCAC |
| | | TATTTCGGTAAGGATATCGCGCACCAGATCTGCTCTGCTTTCAAAAATCGCAGTGAGAACGATCTCGATTTTAAC |
| | | CCTGCCGGCGAAATCGGTGCTTTACTGTACAACCACTTGCCTACCGCACTTGGAGCAGCGAACGGTGATGAACA |
| | | TATTAAGAGCGCGATTTGGAAACGCCATAATCAGGTGCGGCAATTTTACCAAAAGCTGCTCCGAACCAATCGCT |
| | | TTAAGCGAGCATTCCGCACTCGTGACATGGATAAACTGGATAACCTTCTGCCAAAAGATACTGAAAGCTTAACA |
| | | CGGGTTTTGGATGCGAAAGCTGAAAACGCATATCTGAGCGAACTGATTCGGCTTGGTAAAGTCATTGCCCATGC |
| | | GGCTGATATTCCGTTGCCGCCGGTAGATGGGGAGAACTTACAGGATCAGTTCAAAGCCCGGTTGGAGCATTTTG |
| | | CCACCAGCGCAGGTCAAAGCGAAATTAAACGCAACGAGACATTCACCCGCCTGTGGCGGACCTCAACCGCCTTA |
| | | TCGCTGAGAACGTTAACGGCATGGGTGGAACCTATTCTCGGTACCAATGAAAATAACAACCCTCGGAATTTTGA |
| | | TATTACCGATAATAATTTCCGCGGCAGTCTGAATCATCTGTTAGGAGAGAACACACGTGGTGATTTTTCTTGGGA |
| | | CCGTATTAAAGTCATCTTTGGACAAAAAGCATATCCAGCGTTATGTGATATTTCCGATAAAGGCCGTTCTCATAT |
| | | TTTTTCTTCGGGTGAGAGCCAAAATGAATGGCAGGATCACCAGGAAGTCGTCTGGGCGCTTGTCCGCCTGGCCA |
| | | CGCAAATACGCCATGGTACAAATCATTTTAATGTTCGACATCGTCTTGTCGATGAGATCACAGGCGGACTCCTGA |
| | | CGGGAGATAATCATCCACAGCATTTCCATAACAGACCGGGAAATATCGTTCACTCATCAATGCTTGAGCGTTTTG |
| | | ATAAACTGTTAGCGTTCGACACCCGTCTGCAATGGCAGGTTCTGTTAGACGACTTGAACGCGTTTAGCATCACGA |
| | | AATATCTTAATAATCCGCAACTGACAACCTTATTGCAAGAACTCATTAAAGGTCAGACCGGCACAGACTTAGCT |
| | | ACACCAAAATTCATGGCCCTGCTTCGCCGTGCAGCCAACCTGGCGCGGGCCGAAGAGAAAGAATATGGTGAGG |
| | | AAAAAATGCCTGAAGAATCGGAGTTTCTCTCTACAAACGCATTACGTCGTTTTGGCCCGCTTGAACTGACCGATT |
| | | TGTCAAAGAAAAAAGATACTAGCCTGAATGATGTTCGCATTGATCTTTTTCGCCATCTCTATCGTACCGGCTTCC |
| | | CGGCTTGGTTAGAAGAAAAACGCGTGGATGATGATTACCTGAAAAACGCCATCCGGGCTGTTTTTGATGCCCGG |
| | | GCATTCCGTGAGGAAGCATTTGCCGAAGAGCAGCGCATTGTGGCTCAGGGGACATTGTTAGAGTCTTCGGATGT |
| | | AGATGCCATATCTGATCTTAGCGCACTGTTTGCAACCCTGGCGGCAAATGCTGCAGGTGAAGAACGTCTGCGCC |
| | | GTAACTCCCGTGCAAGAGCCAAAACTAGAGAGACTGTAAGAGTTCAGAGTGGCGGCAGACCCGATACGAAAGA |
| | | GAAACCGTCATGGCAGGCCACCCTTAGAGAGGACATCTACGGTCACCTGTTTGCAGAATATTTGAATGAAGAGC |
| | | ACTTAAGCTGGATATGGGATATCAAGGAACCGCATAACCCTATCGCGGAAGATCTGAAACTCGGCGAAATTCCC |
| | | GATTTGGATCAGAGTAACCGAGAAAACTGGGAAAGTCAGTTTTACACCTGGCTTTATCTCGTGCCGAGTGACCA |
| | | GGTTTCCCTGCTCCGTCATCAACTCCACAAGACACTGGCTTTAGATGCAGCGGCCGACAAGTCGGTTTCCGGCTC |
| | | TGCTGCGGATGAGCAAGACCGGGCCACGCTTTCACGCATGTCGAATCTTATGGCGCTTTACACTGCCGTGCAGG |
| | | AAGCGGGTTTTGACGGTAACGAACATGTTGAAGCTTATGGGGAAGAAGCGGACTACATTTTTTATGCGGATCAC |
| | | GATCAATTCAAAAGTATTCATTCTCAAGATGACCTCACGAATACTGTGCCTGGCACGCGCCGTGGGCTGCGACA |
| | | GATCCTGAAGTTCGGTCATCTGTCCGCGGTGCAGCGAATATACGAAAAACACCGGGTAACTCAGGCTGAAGCTG |
| | | AAGACTTAATCGAACTCTATGGAAAAAAACGTGGCGGAGAAGAACCACAAATCAACGACTTCACAAGCCGTTC |
| | | AAAATTACACAAAGAGATCGAAGGCTTGTGGGAAAAACAGCAGCCAAGAGAGAAACACACTACCGACAAAGA |
| | | CATTGAGGATAAGTGTAAGGCGTACAAAACTAGCGCCATTAAAATGGCTCTGCATAATTTTAATATTGCGAGTG |
| | | CTCGCCTGACTGATCACGCAGATCTTCACCACCTGCTGATACGCGTCGTCACACGTCTGCTCGACTTTACGCTTA |
| | | CCTGGGAGCGTGACCGTAATTTCCTCTATCTCGGCATGCTCTGCGATAAGTTACGCACGATTGAAAAAGATGAC |
| | | CGAGGGTCTGTTCTGTCCTCTACTCTCAATCTTGAATTGCATCATTTGCCATACGTTGTGAAAAAGGGTAAAAGT |
| | | GCAGAGAAAAAACGCTGGTCTATCTGTTTAAAAATCGGGGATCGTCACGCTCAAGAATCTGCTGAAATTGGATT |
| | | CAGTGGGAGTGATGAATCAACAATTCAGCAGGCTTTGTTACAGGAGTACGGGCTGCTCCGTTTGTGGGACGACG |
| | | AAATGGGGTTGTCTCTCGATGCTCGGGATATTGAGTTGGATCTCCTGCCAACAGACCACCGATGCCTGTTTATAA |
| | | AGTACTTTGTGAAGTTGGCCGGAGAGAATGAAAAGGATGTTGCAGCCCGAGAAAAAGCACCGAAAAATAAACC |
| | | AATCACTGATCACTCGCAACGTAGCGGCGCTTATTCATATTCATATAAGATATCAAAGAAGCAGATCCGCAATG |
| | | ACTTTGCCCATTTCAATATTCTCACCAAAGAGCGTCTGAATCTCACCTATCTGACCAACGCCGTGCGCTCCCTGG |
| | | TCAGCTATGACCGGAAACTGAAAAATGCGGTTTCAACGGCCATCGCAGATATCGTCTCAGACGCTGGTCTGGAG |
| | | ATTCAGTGGACGTTGGAGCGGGACCGGCTGAAAAAGCCGTTGGTGATACCTAGAGTAGTCCACCACTTAGCATG |
| | | GTTACCCAAAATCAATGGGGAAGATGTATCCTTTTATCTTCCGGCTAGATCCGCACGGTTTACCTCAATGGTAAA |
| | | GGCGCTGTTTGATTTCGGCACGGAAGGCAATGTGGTTGAAATCGAGAAAGACGGTACGGGTATAACAGGAAGA |
| | | CTTGAATATCCTAGCGCGTTAGCGGAGCGCTATACCATTACAAAAACAGCAACAGACGAAGCCGGTGACGGTG |
| | | GCGAGATTCCCGAGACACCAGCGCAGATTTGTTTAATTCCGTCTGAAATTCTGACGTCGCTGGAAAATCTGCCTC |
| | | AGGAAGAAAGTAAAACTATGATAAAAAATAGACGGTAA |
| 303 | JW13126 | ATGAATCATACAATTGGTAAAGGCATATCATACAACCCCTTCAAACACCAGGACAAACACTATTTTGGATCGTT |
| | | TTTCAATTTGGCCGACAATAATATTAAAGACGTGTTTAATGAAATCGCCCAACGATTGAATCACAAAGATTCAA |
| | | ACCCGAAAAACATTATTAACCACTATACGGATAAAAATATCAGCCTCGTTGACTTTGAAAAATTCGTATCCCTGC |
| | | TTTCTAACTATTTCCCGATAGTTAATGAAATTGACCAAATTAACTGTGATGCTAAAGGTTCTAATGGGAATTATA |
| | | AACCGAAAACTAAGGAAGAAAGATTAGGCTACTTCAAAAAAACTAGTTACAGTCTGCTGGAAGCCATTGATAA |
| | | ACTGCGGAATTACTATACGCATTATCATCATGAACCCATCGAAATAGATCTGAACCTTTTCCCGTTTTTAGATCA |
| | | TGTGCTGCTGAAAACAGCTTTAAACACTAAAAAGAATTACCTGAAAAAGGGCAAAACCAAAGAGTTAATCAAG |
| | | GACAATCTGAAGCAGCAGTTTGACTTTATTTTCAACATCAAAGTTGAGGAATATCTGAAGGAAAAGAACCCTAC |
| | | ATCAAAGAAAATTGAAAGAGCGGGCGGTAAAATCGAAAATTTCAATAAAAGCTTAGTCGAAGAGATAACATCC |
| | | GCGATCTACAATGATGCTATCAAAGATTTCATCTACTCAAAAGGTGAAAATGCAGAACTGAGTAACCGTAAAAA |
| | | AACGAGCTTTTCAGACAAAGATTATTTTTATTACAACGATGACGATCACTTCTCTCTGCCGATTAGTTCTTCCGGT |
| | | ATAGTGTTTCTCTTGTCTTGTTTTCTGAACCGGAAAGAGATTGAATCCTTAAAAGATAACATCACCGGTTATAAA |
| | | GGTAAGATACCCAATAGCGAATTAACATTAAAACATAACGGTATTCGTTTTATGACAACGCATCGGATCTACTC |
| | | TCATTGGTGCTATAAAGGATTAAAAACCAAAATACGTACAAGTGAAAAAGCCACACGAGAAACGCTCTTGTTAC |
| | | AGATGGTGGACGAATTATCTAAAGTTCCAGATGTCGTCTATCAAAATATTAGTGATGACCTGAAAAAAACCTTC |
| | | ATCAAAGATCTTAACGAATATTACATTGACAATCAGGAAAACCAGGAGAACCTTTATAATTCTAAAGTTGTTCA |
| | | TCCCGTAATTCGTAAGAGATATGAAGATAAGTTCAATTATTTTGCCGTACGCTTCTTAGACGAATTTTTAGATTTT |
| | | CCAACATTGCGTTTTCAGGTCCATCTTGGCAATTATCTCGAGCACAGCACCTCGAAAAATATTGGTAACAGTAAA |
| | | ACGAACCGTGAAATCAAGAAGAAAATTTTTGTTTTCGGCAAACTTTCCGAAGTCAATAAGCTGAAGAACGATTT |
| | | CTTTGAGAAAAATAAGCAGAACGATACCTCATGGCAGTTTTTTCCAAATCCTTCGTATCACTTTCCGATGGAAAA |
| | | TTCGAACGACTTGAAAAAAGCGAATAAAATAGGCATCTACATAAACCATGACAAAAGCATTAACAAATTCAAA |
| | | CATCAAACCCAGAAGCAGACCCATGAGAATAAAATTAAGCTGATTCAAAAAGTAATCGGCGATAAGAGTAAGA |
| | | TTAAAATTGGTCAACCAATAGCATATCTGTCTATGAATGATATACATTCGATACTGTTTGAAGCATTAAAGAACC |
| | | CGAAAATTAAAGAGTCAGGAAAGATCGATGGAGAACATATTGAGTATAAGATCACGCATCAGATTAATAAGCA |
| | | GATTAAGGAAATACAGAACAAAAACGAGAACGCGAAAATCATTAAAAATCAGCAGTCAAAACAAGAGACGGA |
| | | CTTTAACATTGACAAGCTGATTAAAGACGTCAATAAAGAGATAACTATATCAGAAGATAAGATCCAAACGCTGA |
| | | AAGAAAATAAAAAACAGTATCAGAATTATCAACGCTCCAAGAAATCAGAAAAACACAATAACTCACGGAAAAG |
| | | AAATCATATTCTGTATAATAGTGAAAAAGGGGAAATTGCGACTTGGCTTGCGAATGATATCAAACGCTTTTTTA |
| | | GTAAAGATTTCAAAGACAAGTGGAAAGGGCACCACCACAGTAACTTTCAACGTAATCTTGCATACTATAATACG |
| | | CAAAAAAAAGAGGTGGAGCTCCTTCTGAAAGGTCTGGATTACCGCAAAACTATCCCGATGCTTGATTTTTCAAA |
| | | GAATTCTTTCCTGGACTTTTATCGCAAGTACCTGGAGAAACGCAAACAGTATTTTAAAAACTTGTTATCCGAGCT |
| | | CAAAAATCATAAAAGTGGGAAACACGATTCTATCGAGAAATTGAAGAAGAAATGCTTTACTCACTTCAAAAAG |
| | | CGTAACTATCGAAACAAAACTATAGAAGACAAAATTCAGAATATGCTGGCCAATCCTATCTTTATTAAACGCGG |
| | | CTTTATGGATACGAAACCTACGATGATTCCTGGGAAGAATTTCAATCAGAATAAAAGCGATTTTGCTGACTGGTT |
| | | TGTGGCTTTTAAAGAATTCTCGGAATACCAAAAATTTTATGATACCAATCTTTACCCTATCGATTTGAGCCAGAA |
| | | ATCCAAGAGCGCAACAAAAAAAATTAACACTGAGATCTACAAACAGAAAAAGAACGATTGGGCGATCTGGCAG |
| | | ATGACTAAGTATATTTTCAAAGACATTTTCCAACAGGACTCTCAGAATATCTCACTTTCGGAAATCTACCAGTCC |
| | | CGCCAAGAACCTATTAGCAACGCAAAAAATACCGCTACCGATAAACGTAAACAGAATTCTATCTGGGATAGCAC |
| | | AATTGATCTGCAGCTTAACCGGAAAATTAAAATAAGCGATGTGAAACTGAAGGATATTGGGAACTTTCGCAAGT |
| | | ACGAGAATGATCAGCGTATTAATACCTTTCTGGACTATGGAGATTTTAGTGATTGGATGGCCTATCTCCCAAATA |
| | | ACTGGCAAGAAAATTTTGAGTATAAGCCCATCAATGTTATTGATATCCAACTGGATGATTATGAGAAAATTCGT |
| | | CAGAACGTTTTATTGCAAGAGGTGCAGAATTTGGAACGGGAGATCTATAATAACGTAAGCAATAAGAATGAAA |
| | | TACTCGTGCAGGACTCGAAAGGAAATAAAAACCCGAATTTTAAAGCCTACATACTGAAGGGCTTATTGAAGCAA |
| | | ATTAAGAACCTTGATATAACTAATTTTAAACTTCCGCAGAATGCTAAAGAATTCGATACCCTCACCTTTAAGCAT |
| | | TTGCAGACTCTGAGCGTAATTGAGCAAAAAACCACCCTGCTGATTTTACTGAGAAACAAATTTGCACATAACCA |
| | | GTTACCAAATAAAGCAGCCTATGAATTTTGTCAGACAATTCTGGAGCGAGAAAAGTCCCAAACGTATGCGCATT |
| | | ATTATAGCGAACTGTTCAAGACCCTCAAAAAAGAGTTAACCGATATATGA |
| 304 | JW13127 | ATGGAAAATACTATTGAGATACAAAAACAATCCATACGTACATTAAACAATGCACCGCAATATTTTGGCCTGTA |
| | | TCTTAACATGGCGAGACTGAACATTTTTAATATTTCGAACTATCTCGCGGAAAAATTTGGTCTGGCAGTGTTAGA |
| | | TGAAGAAGAGAAAATAACCATTTCATTCCTTGGCGATAAAGGCAATAAAGAATACAAGACCAAGCAGTCTTAC |
| | | ATCTACTTTAATCTTATCAAGTATATGCCTGTCGCAAAAGTGTTTGATTTTGAAAAGCTGCCAAAGAATGAGATT |
| | | GACAAAACAAAAAACAATTCAGGGAAAGACTTCAACAAACTGTCAGACACCCTTAAAGTAATTTTTAAAGAAC |
| | | TCAACGAATTTCGCAATGATTACTCTCACTACTTTTCCACTGAGAAACAAGATAAACGTAAGCTTAAGATTAGTT |
| | | CCGAGCTTAAAGATTTCCTTAATATTAACTTTAGCCGCGCGTTATCGTATACCAAGAATCGTCTGAAAGATGTCT |
| | | TTAGCGATGATGACTTCAACATTGCATCTCAAACAGTTGTTGTTAATAATGATAATACCATCACACAGGATGGA |
| | | ATAGTGTTTCTGATCTCGATGTTTTTGGACATGGAAAATGCATTTCAGTTCATCAATAAAATCATAGGGCTGAAA |
| | | GGCACCCAGACTAAAGCCTTTAAAGCTAAACGTGAAGTTCTGATGACATTTTGTGTGAAACTGCCACACGATAA |
| | | GTTCGTATCGGAAAACCCGCAGCAGACATTTTCGTTAGAGCTCATCAATGAGCTGAACAAATGTCCGAAGCTGC |
| | | TTTACAATGTGATTACAGAAGAGGAAAAACAGATTTTCAAACCGCAGATTCAAACAGAACAGATTGAGAACATT |
| | | ATATCCAACAGCGTCCCGGATACCATTGATGATTACGAAAACTATATCGAGAGTATAACTAAAAAAGTGAGATA |
| | | CGAAAATCGCTTCTCCTATTTTGCACTGAAATATATTGACCTCAAAGAACTGTTCAAAAAATGGCGGTTTCAAAT |
| | | TGACCTGGGAAAAGTTGTATTAGACAAGTATAGCAAACAGCTGAACGGTAAAGATGAAGTCCGCACGGTCACT |
| | | GAAAATGCGAAGGCTTTTGGTCGTCTGCAGGACTTTTATGATCAGGAAAAGATCCTGAACAAAATCAACATCGG |
| | | GGAAAATAAAACTCGGTTTTATCAGTTCGCCCCACATTATAATTTTGACGTGCAGAATAATAAAATCGGGATTA |
| | | GTAAGAAAGAGACGACGGCGAAATTCATCTCAAAGAACACAACGCACAAAGTTAAGTTCAATTTGCTGCAACC |
| | | ATTGCCTGAGGTTTTTTTAAGCATTCACGAATTGCCGAAAATTATTCTTCTGGAGTATCTTGAACAGGGAAAAAC |
| | | GGAGAAATTGATCAATAACTTTATAAAGCTCTCTAACAGTACCATCCTCAACAAAAAATTCATTGAAACGATCA |
| | | AGTACAAACTTTCAGATCTTGATGTCTTTCATAAACGCTCCATGGGTAGAAAAGAAATTTCTGCTTATCGCAAAG |
| | | ACATTCTGAAGTATCTGATCGCCCGTAAAGAAAAACTCAACCAGATTTTGTCTGAACATAACCTGAGTGTTAAA |
| | | CAGATACCCACTCGCATACTCGATTATTGGTTGAACATTGCAGATGTTCAGGAAAAAACCGCCATTAGTGATCG |
| | | AATAAAACTTATGAAACGAGATTGTATGGACCGTTTAAAAGCCATTAAAAAAAATAATGCGCCGAAAATTGGA |
| | | GAGATGGCTACGTTTTTAGCAAAGGACATCGTAGACATGCTGATATCTGAAGGTAAGAAGAAGAAAATCACTA |
| | | GCTTTTATTACGATAAAATGCAGGAATGCCTTGTACTGTTCGCTGACAAAGAGAAGAAAGAATTATTCATATCA |
| | | ATATGCAGCGAGTTAAAGTTAAACGACGCCGATGGTCATCCATTCTTAAAGAATATCAATCTCAGTGATATTAG |
| | | ATATACCTCTGATTTGTACAAGATATACCTGCAAGAAAAAGCCGATAATCGGAATCAGCGTAATCGTACCGATA |
| | | CGTCTTGGCTGGCTAAAAACTTTTACACGTTGAAAAATGTTGAGGTGTTCGATAAAAAACAGCAACGTAAGATT |
| | | TTTAAGCTTCAGACCATTGTTGAATTGCCGCAGGATAAAAGTAAAATTCCGTTTACTATTCGACAATTGGAGAAG |
| | | GAGAAATCAAGCTTTAATAAGTGGTTAGAATATATCACCAAAGGTAAAGAGACCACAGATAAGAAAAAACCCG |
| | | TGAATCTGCCCGCAAATCTTTTTGATGACACGTTAAAAAAACTGCTGAAAACAGAGTTGTCAAAAAACAACATC |
| | | CAATACAACAAAGAAGCCAACTATAATCAACTTTTTAAAATATGGTGGAAAGAGTTTCGTAAAGACAATACCCA |
| | | AAATTTCTATACTGCGGAGCGCGAATATACTTTCGAAGAAGAAAAATTGAACTTTAAGATCAATACGCAGCCAA |
| | | AGTTTGAGAATTATTATACTCGCTTCGCGGAGAATGTCTTTAATAAAAAACAGGCGTTACGCAATGAAGAGCGT |
| | | AAAACGAAACGGAATCTGCCGCCTATTCAGAAAATCCATGTAATTAAGAAGATACGCAACAAAATAGGAAATA |
| | | CGGAAAAGGAAATTCGGATGCTGCAAGAAGAAGACCGTTTAATGTTGTTAATGTTTGAAAAACTCACAAAAGA |
| | | GAATCTGAATCTCAAACTGGAAAAGGTTAACGAGTTACTCAACGAACGGATCGAAATTAAGGAAAAAATACCT |
| | | GGTAAACTGTCATTTGATGATCTTGGCGAGAAAACAATAACTAGAAATATTGTGGAAACCCGGAAACGCAAAG |
| | | AATATTCCGTGCTGCAGAAATATATCTTTGACAGACGATTGCCTGAATTATTCGAATATTTTAATGAGACAGATA |
| | | TACTGATCGAGAAGATCAAAACCGAACTGGATGCTTACAACAAAGCCAAAGATATTGTATTCGATCTGATTTTC |
| | | AAACTGGAAGAGGCTATTATTAAGAAGGACGAAACAGGTATTAAAGAACTGAACAAAGATCAAAATGGCAATA |
| | | TCCAGACAGGCAATATTCAGCATAAACCCTATCTTCATTGGCTGCTTAAAAAGAATATGATTACCGAGAAGGAA |
| | | TTCACCTTCATTAATATGGTCCGTAATACTTTTAGCCATAATCGTTTTCCTCAAAAGAAAACGATGGAACTTTTC |
| | | ATCAAAAAATGGAACGACAACAATTTTGCCTATCAGATCCTGGAAACTTACAAGCAGAAAACCAACAACATCAT |
| | | TGAAAAATTAAAAGATATGTAA |
| 305 | JW13128 | ATGAAAAAGAAAAGTAATATAAAGATCATGGGCGTTAAAAGTGTACACTATTTCAATAATGACGTAATTATCGA |
| | | GAATTCACTTAGTGAAAATGTCGAAATTTTGTTCAAACCAAATTACCAAAAAATAGACGAGTTAAAAATCCTGA |
| | | ACTCAAATACGAATAATCAGACAAATAACCGGGTTGAATTCATCAATGATAATGAGAAAAAAGGCTTTACCCTG |
| | | AAAAAGAAAGATTCTTCGTTTGAGAAAGATAAAGCACCGAAAAGTATTAATTCGTACGTCAATAAGGTAAAAA |
| | | ACAACAGCTTTATTCACAATGAGTTATTCGGAAACAAGAATAATAACTTTATTCCGCAGATCTATAGCAATTTTG |
| | | AGGATATACGTAAAATCATACTGAACTACATTGATCATATCATCTATTCGGTGAATAACAAAATTATAGCTAAC |
| | | AAGTGCAAAATTGAAGTTGATCGTTTATCTTTTGATGATATTGGTATGTTACCTGCAACCAAAACTTATGAAGAG |
| | | TATGGCCAGACACCGGAACAGCGCGAAAATATGACTGATGTGGTTCTGCCCGCCTTTAAAGAAGTACTGAAAGA |
| | | TAATAATATCAAAACGGGCAATTACGGTAATATCAAAGTTGATTACTGGAACATTGTTAAGTACGCTTACAAAA |
| | | AATCTAGATGTCAGGAATATACTATACGCGAACTTAGCAATGACTCTTTAAACTATATTAACAATAACATCAAT |
| | | GCGATTGCCGCTTATAGTAAAAAAGTGAAAGACTATCCGTTACTCAACGAACTTAACTTAGATCGCAATAAGTA |
| | | TATTATTCATCTCGAGCGCAAAAATGAGAATGAAAAAACTGCCATTAAAAAGCTGAACAATCATTTTAGTGGGG |
| | | AAGAAATCTTTAAAGAAATCTATGGAATCGAAAAAGAATTCCAGGAAAAAAAGGAATTATACAATTATCTCCGT |
| | | ATCATCTCTTTAATTCGCCAATATTGTGCGCATTTTATTACGTTTGATAGCGAAGAGGATTCAATTAAAGCCCTT |
| | | ATAAACCCCTCTAACAATCTTATCAAGAACTTTAAGACATCCTTCATAAAAAACAACGAAAAGTATCTGCTGGT |
| | | ACTTTATGAATATCAGGATTTCTATTCTGAGCTGCTGAACAAAAAAAACATTAACAAGGAGAACAACATCTTCA |
| | | CTGAGTTTTACAATTACTTAATAAAAGATGAGGCGAAAAACCTTGGTATTTCCGTTGATAAACTTCAAAAGCGG |
| | | TATGTCTTTAATTTGAATATAGAGGACGACACAAAAAAAGCGGAATATCTGAACAAAATGAAAGTCCTGATCAA |
| | | GTTTTTTATTACGCTCGAGTTGAAATACGATAAGGATTTCAATAAAAATGACAGCTCCGATGTGAATTCCAAATT |
| | | CTATTATATCGAAAAAATTAAAACCGCTAAGTACGCGAATAACGAAGATATTATCAAAGCCTTGAGCGATAAGT |
| | | ATAGCGATTTTTCTATTGATGATTACAAAGACGAATACGATTCTCTCTATAACCAGTCTAAAGATAAGATCTATG |
| | | ACGAATTATATGAAAAACTCAACGTTAAGATTGACACCGATAAACTTTCTGATTATGTGATTGATATTCTTGGTA |
| | | AAGAAGAAAACAACATTATCAAGAAGTTCACGTTTGACAATCTGAAAGTCAAATATTCTAAAATTGAGGAAGA |
| | | GGTGAATGAGTACAAGAAATGGGAAAAGATTAAAAACAAAGAGAAAAAGGAGATTTTTGAGAATCAGATACGT |
| | | GATCTGTTTCCCGACATTTTTAAGCATATTAACGAGTACAATGAGGAGTATGAAAAAAACTTCAAAAACAAGAT |
| | | TAAACTGCCTAATATATCCACCGATTCGAAGTTTTTCAATCTCATCTATTTTGTTTCTGAATTTCTGACTACTAAA |
| | | GACGCAAATAATTTGTTTTCAACCATATTGACCAAACTTGACTCGATTAGTAGCCTCTTAAGTCTGTTAAACTCG |
| | | TATGACAGTAATAGCAATATAATAGGCAATGAGTTCATTGATTACAAGTTTATGTTGGACAACAAGGAAATTAA |
| | | CATTGAGAACATTTGCAATTTCAAGGATGATATAAAGATACTGAAAAGCCTGCGTATAAAGGATAATACGAAAA |
| | | CAAGCAAAGCTAATATTAACGACGTCGATTTTAACAACATCTTTAATTCATTCAACGATGCAGGAATGTCACTG |
| | | GAAAAATTCAATCGGAAAATTGAATTAACCACTGAATCCCAAGAAGATGATTATGTTGCTAATAACTACAAGGA |
| | | GTTAGTTAAGTATAATGCCCTGTCATCCTACAAAAAGTCCGTCAATGAACTTAATAATAAGAAGTACAACAAAA |
| | | AAACGTTGCTCGATCTGAAAGACGAAGATTTGAAAGAACGAATCAATAACTATTTGTCCACCTTAGACCTTTTC |
| | | AAGAATTATAACTGCGACAAAAATTACATAAAACTGGTGAAAGAAGCCGTGAACTACAAAAAAAAGGAAAAAC |
| | | CTTTGAAACAATACCTTCGAAATTCTATCTATTCGTCAAAACAATACCAATACATAAAGGATTATGCGAATACAC |
| | | GGCTGTGCAAATACATCATTGCAAACGAAAAAATTCTGGACTATATCTTAAACGATAAGTTTGATACAGAAGGC |
| | | ATGAAAAAACATCTGTATCGCGTATATCTGGATTACAAAAAAGAGGAGATTACTAAGGACAAAATTGACAACA |
| | | ATTACAATAACGTGAACATTAATGAACTCAAGAAAGCCCTTCAGGAAATTAACCTGAAAACGCTGAGTGATAAT |
| | | ATTCTGAAGAATATACCAGAGGACAGAAATTTGAAAGCAATTATAGGTCTTTATTTTGAGGTATGTTACATTGTG |
| | | ATTAAAGGGTTGTATGACGTGAATTCCGCCTATATGATACAGATACAGGAATATGAGAAAATCTACAAAAAATG |
| | | TTTCGGAGCGAGCAATTTTTCATTCAACACCGAATTACTGAAGAAGTTTGAAGAATATGAAAAAAATCGAAACC |
| | | CAGAATCAAAAAGTCTGAAACAGATCAAAGGAATAATTGGTGATGGGAAATTTGGTCACGAATATGTGCGTGA |
| | | GGAAACAGATGATGATGAAAAAAAGAAGAAAGCACTGATCTATATTCGTTTTCGCAATACAGTTGAACATAGCT |
| | | CATTGCTGGCGAATAGTGACATCCTCAAAAACTTTAATGAGGACGAAAGCTTTACGAAAAACGAATTCAAAATC |
| | | ACAAGCATGTTTCAACTGTACCACATCCTGATTCAGCGTTTACTGAAAAAAGACAGTTCAAATAACAAAGACAA |
| | | AGATGGTAATCCTAATCCTTATTATCCGATTGGTAGTGAATTTGAGCCGGATAAAAAAGGCTATTCGACCAAAG |
| | | CTCTGATCATTATGAATCTTCCGTATCTGTATAATGTTGCAAGATTCAATAATCTTACCATCGAAAAATACTATCT |
| | | TCGTAGATTTGAAGGGAATGTCAAGAATAAAAAAGAACCAAATTCATCCTCAGAATAA |
| 306 | JW13129 | ATGGGTATAGACTATTCTCTGACTAGCGATTGCTATCGCGGTATTAATAAAAGTTGCTTTGCGGTTGCCTTGAAC |
| | | ATTGCATACGACAATTGCGACCATAAAGGCTGTCGTACATTGCTGAGCGAAGTCTTACGTTCAAAAGGTGGAAT |
| | | TAGTGACGAACAGATCAAAAGTCAAGTGGTAGACGGAATACAGAAGCGGCTGAAAGATATCCGAAACTATTTT |
| | | TCTCACTATTATCATGCAGAAGATTGCCTGCGATTCGGCGATCAAGATGCAGTCAAAGTATTTTTAGAGGAGATC |
| | | TACAAAAATGCTGAAAGCAAGACTGTAGGCGCGACTAAAGAATCAGACTATAAAGGTGTAGTCCCGCCGCTGTT |
| | | TGAATTGCACAATGGAACGTATATGATTACTGCTGCGGGAGTAATTTTTCTCGCGTCGTTCTTCTGTCATAGATC |
| | | TAATGTGTATCGCATGTTAGGTGCCGTGAAAGGCTTTAAACATACCGGCAAGGAACAGTTATCAGATGGGCAAA |
| | | AAAGAGATTACGGCTTTACTCGTAGACTGCTGGCGTATTATGCACTTCGAGATTCATATTCTGTTGGCGCAGAAG |
| | | ATAAAACGCGTTGTTTTCGAGAAATTCTGTCGTATCTTAGTCGTGTACCCCAATTAGCCGTTGATTGGTTGAACG |
| | | AACAGCAGCTGTTAACCCCTGAAGAGAAGGAAGCGTTTCTGAACCAGCCTGCGGAAGATGAAGGCGGCGATAT |
| | | TTCCGATTCGTCTAGTTCAGACAAGAATAAAAAATCTAAAGAGAAAAGACGTAGCCTGCGCCGTGATGAAAAAT |
| | | TCATCCTGTTTGCTATTCAGTTCATAGAGGGCTGGGCCGCTGAGCAAGGATTGGATGTTACATTTGCACGCTATC |
| | | AGAAAACTGTTGAGAAAGCTGAAAATAAAAATCAAGATGGCAAACAGGCCCGGGCTGTGCAGCTGAAATACCG |
| | | GAACCAAGGGTTAAACCCAGACTTCAACAATGAATGGATGTATTACATCCAGAATGAACACGCCATCATCCAGA |
| | | TCAAGCTTAATAATAAGAAGGCGGTGGCGGCGCGTATATCCGAAAACGAACTGAAGTATTTGGTTCTTCTGATC |
| | | TTTGAGGAGAAAGGTAATGATGCTGTTCAGAAACTGAACTGTTATATCTACAGTATGAGCCAGAAAATTGAGGG |
| | | AGAATGGAAACACCGACCTGAAGATGAACGGTGGATGCCGTCCTTTACAAAACGCGCTGATCGTACAGTTACCC |
| | | CCGAAGCGGTGCAATCACGGCTGTCTTATATTCGCAAGCAGCTGCAAGAAACCATTGAGAAAATTGGCCAGGAA |
| | | GAACCGAGAAATAACAAGTGGTTAATCTACAAAGGTAAGAAAATATCAATGATCCTGAAATTCATATCCGACTC |
| | | TATTCGCGACATCCAACGCCGTCCAAATGTAAAACAATATCATATTCTGCGGGATGCACTTCAGCGCTTGGACTT |
| | | TGACGGCTTCTACAAGGAATTGCAAAATTATGTGAATGATGGTCGGATAGCAGTTAGCCTGTACGACCAAATAA |
| | | AAGGTGTGAATGACATTTCGGGGCTGTGCAAGAAAGTCTGCGAGCTCACCCTCGAGCGGCTTGCCGGTCTTGAG |
| | | GCTAAAAATGGTTCGGAACTTCGTCGTTATATTGGGTTAGAAGCCCAAGAAAAACATCCAAAGTATGGTGAATG |
| | | GAACACGCTTCAGGAGAAAGCAAAACGCTTCCTCGAATCTCAATTTTCCATCGGAAAGAATTTCTTGCGCAAAA |
| | | TGTTCTACGGGGATTGTTGTCAGAAACGGTGTTTTGATGAGGAAAAAGGTTACAACACCCAGGCAAAAGAACGA |
| | | AAAAGCCTCTACAGTATCGTCAAAGAGAAGTTAAAAGACATTAAGCCAATTCACGATGATCGGTGGTATCTCAT |
| | | TGACCGCAATCCGAAAAACTACGACAACAAACACTCCCGTATTATTCGCCAGATGTGTAATACCTACATTCAGG |
| | | ACGTTCTCTGCATGAAAATGGCGATGTGGCATTATGAAAAGCTGATTAGCGCCACGGAGTTTCGTAACAAACTT |
| | | GAGTGGAATTGTATTGGGCAGGGTAATATGGGATACGAGCGCTATAGTCTGTGGTATAAGACCGGCTGTGGAGT |
| | | CGTCATCCAGTTCACGCCCGCCGATTTTCTGCGTTTAGATATTATAGAAAAACCGGCAATGATTGAGAATATCTG |
| | | CCAGTGCTTCGTGCTGGGGAACAAAAAACTTAACAGCGGTGCCGAGAAAAAGATTACATGGGATAAGTTTAAT |
| | | AAAGACGGGATAGCGAAGTATAGAAAAAGACAAGCTGAAGCAGTGCGTGCAATTTTTGCTTTTGAAGAAGGGC |
| | | TGAAAATACAGGAAGATAAGTGGTCCCATGAACGTTATTTTCCGTTTTGCAATATATTAGATGAGGCCGTTAAA |
| | | CAGGGTAAGATCAAAGATACGGGCAAAGATAAAGAGGCATTAAACCGCGGTCGTAACGATTTTTTTCATGAAG |
| | | AATTCAAGTCAACAGAGGATCAGCAGGCCATCTTTCAGAAATATTTCCCTATAGTGGAAAGAAAAGATGACACA |
| | | AAAAAACGTCGCGATAAGAAACAGAAATAA |
| 307 | JW13130 | ATGGAGAACCAGACACAGAAAGGCAAAGGCATCTATTACTATTATACCAAAAATGAGGACAAGCACTATTTTG |
| | | GAAGTTTTCTCAACTTGGCTAATAACAATATTGAGCAAATCATTGAGGAATTTCGTATTCGTTTAAGTCTGAAAG |
| | | ATGAGAAGAACATCAAAGAGATTATCAATAACTATTTTACAGACAAGAAATCGTATACTGATTGGGAAAGAGG |
| | | AATTAATATATTAAAAGAGTATCTGCCAGTTATTGATTACTTGGACCTTGCCATTACAGATAAAGAATTTGAAAA |
| | | AATCGATCTGAAGCAAAAGGAAACCGCCAAACGGAAATACTTTCGTACGAATTTTAGTCTGCTGATTGATACGA |
| | | TTATTGATCTCAGAAACTTTTACACTCACTATTTCCACAAGCCGATATCCATCAATCCAGATGTTGCAAAATTCC |
| | | TGGACAAAAACCTTCTGAACGTGTGTCTGGACATTAAAAAACAAAAGATGAAGACAGATAAGACCAAACAGGC |
| | | GCTTAAAGACGGTCTGGATAAGGAACTGAAAAAATTGATAGAACTGAAGAAAGCGGAGCTGAAGGAAAAAAA |
| | | GATAAAAACCTGGAACATAACTGAAAACGTCGAGGGGGCAGTCTATAATGATGCCTTTAATCACATGGTTTATA |
| | | AGAATAATGCAGGGGTAACAATTCTCAAAGATTACCATAAAAGTATCCTTCCGGATGACAAAATTGACAGCGAG |
| | | TTGAAGCTGAACTTTTCAATATCTGGTTTAGTGTTCCTGCTGTCTATGTTTCTTTCCAAAAAGGAAATTGAACAAT |
| | | TCAAATCGAATCTCGAGGGTTTTAAAGGTAAAGTGATTGGAGAAAATGGCGAGTATGAAATAAGTAAGTTCAAT |
| | | AACTCATTAAAGTACATGGCAACTCATTGGATATTTTCATATCTTACGTTCAAAGGCCTGAAACAACGCGTTAAA |
| | | AACACTTTTGATAAAGAAACGCTGCTTATGCAGATGATTGATGAATTAAACAAAGTTCCTCATGAAGTTTATCA |
| | | AACCTTGAGCAAAGAACAGCAAAATGAATTCCTGGAAGACATTAACGAATATGTGCAAGATAATGAAGAAAAT |
| | | AAGAAATCCATGGAAAATTCCATTGTGGTGCATCCAGTAATCAGAAAACGCTATGATGACAAGTTTAATTATTT |
| | | TGCCATTCGATTTCTTGACGAATTTGCTAACTTCCCGACCCTGAAATTTTTTGTAACTGCCGGTAATTTTGTGCAC |
| | | GATAAAAGAGAAAAACAGATTCAGGGTAGCATGTTAACGAGCGACCGGATGATCAAAGAAAAAATTAATGTGT |
| | | TTGGTAAGCTGACAGAAATTGCAAAGTACAAGTCGGACTATTTCTCCAATGAAAATACTTTGGAGACATCGGAA |
| | | TGGGAATTGTTTCCGAATCCGTCCTACCTTTTGATACAAAATAATATTCCTGTACACATCGATCTGATCCATAAC |
| | | ACCGAAGAGGCTAAACAGTGCCAGATAGCGATAGATCGCATAAAATGCACAACTAATCCGGCAAAAAAACGTA |
| | | ATACACGGAAGAGCAAGGAAGAGATCATCAAAATCATCTATCAGAAAAACAAAAACATTAAGTACGGAGATCC |
| | | CACGGCACTCCTCTCAAGTAATGAGCTGCCTGCGTTGATCTACGAATTGTTAGTTAATAAGAAATCGGGTAAAG |
| | | AATTGGAGAATATTATTGTCGAGAAAATTGTGAATCAGTATAAGACTATAGCTGGGTTCGAGAAAGGTCAGAAT |
| | | TTGTCTAACTCTCTCATAACAAAAAAATTAAAGAAATCAGAGCCCAACGAAGACAAAATCAACGCGGAGAAGA |
| | | TTATTCTCGCGATTAATCGTGAGCTCGAAATCACGGAAAACAAATTGAATATTATTAAAAACAATCGCGCAGAA |
| | | TTCCGCACCGGCGCCAAACGTAAACATATTTTTTATTCAAAAGAACTGGGTCAAGAAGCCACGTGGATTGCTTA |
| | | CGATCTGAAACGCTTTATGCCCGAAGCTAGCCGTAAAGAGTGGAAAGGGTTTCATCATTCAGAACTGCAAAAAT |
| | | TTTTAGCCTTTTATGACCGGAACAAAAATGATGCGAAAGCCTTATTGAACATGTTTTGGAACTTCGATAATGATC |
| | | AATTAATCGGGAATGACTTGAATTCTGCTTTTCGTGAATTTCATTTTGACAAATTCTATGAAAAGTACCTTATTA |
| | | AGCGCGATGAGATACTGGAAGGTTTCAAAAGCTTTATCAGCAACTTTAAAGATGAGCCTAAACTGTTAAAAAAA |
| | | GGCATTAAAGACATCTACCGAGTCTTTGATAAACGCTATTACATTATTAAGTCTACCAACGCACAGAAAGAGCA |
| | | GTTACTGTCAAAACCAATCTGTTTACCGCGCGGGATTTTTGATAATAAACCTACTTATATTGAGGGCGTGAAAGT |
| | | AGAAAGCAATTCTGCCTTATTTGCAGATTGGTATCAGTACACCTATTCCGATAAACATGAATTCCAATCCTTTTA |
| | | TGATATGCCGCGTGATTATAAGGAACAGTTCGAAAAATTCGAACTGAATAACATCAAGTCAATACAGAACAAG |
| | | AAAAATCTTAACAAGAGTGACAAGTTCATCTACTTTAGATATAAACAGGATCTGAAAATTAAACAGATCAAAAG |
| | | TCAGGATCTGTTCATAAAACTGATGGTTGATGAGCTTTTCAATGTCGTGTTTAAGAACAATATCGAGCTGAACTT |
| | | AAAAAAACTTTACCAAACCTCAGACGAACGTTTTAAAAATCAGCTTATTGCTGATGTCCAAAAGAATCGTGAAA |
| | | AAGGCGACACAAGCGATAATAAAATGAACGAAAATTTTATCTGGAACATGACGATACCGCTGAGCCTCTGTAAT |
| | | GGCCAGATTGAGGAACCTAAAGTTAAACTTAAAGATATCGGTAAGTTTCGGAAACTTGAAACAGATGATAAAGT |
| | | TATCCAGCTGCTGGAGTATGATAAAAGTAAAGTTTGGAAGAAATTAGAAATAGAGGATGAGTTAGAAAACATG |
| | | CCGAATTCTTATGAACGGATTCGACGAGAAAAATTGCTGAAAGGAATTCAGGAGTTCGAACATTTCCTGTTAGA |
| | | GAAAGAGAAATTTGACGGAATTAATCATCCAAAACACTTCGAACAGGACCTCAATCCAAATTTTAAAACCTATG |
| | | TTATAAACGGAGTATTACGCAAGAACTCAAAACTGAATTACACGGAAATAGACAAGCTTTTAGATCTGGAACAT |
| | | ATCAGTATTAAAGATATAGAAACCAGCGCGAAAGAAATCCATCTGGCTTATTTTCTGATCCATGTCCGTAATAA |
| | | GTTTGGCCACAACCAGCTGCCCAAACTCGAAGCGTTCGAGCTGATGAAAAAATATTATAAGAAGAACAATGAG |
| | | GAAACCTATGCGGAGTACTTCCACAAAGTATCTTCGCAGATTGTCAATGAATTCAAAAATAGTTTAGAAAAACA |
| | | TTCTTAA |
| 308 | JW13131 | ATGAATAATAATATTTTAGAACAGTCCACTAGCGGTTCTGAAAAACGCGCATCAACACGCACATTGGAAGCTGA |
| | | TCCTCAATATTTTGGGGTCTATATCAATATGGCACGTCACAATGTCTTCCAGATTATTAATGCCCTTACGCACAA |
| | | GTTTAGTAAGCAAATTGAGAACGCAAAGCCGATTAACGACGATGAAAACATCGCTAACGATTATCTGATCAATA |
| | | TTGGTAAAGGGACCGACAAAAGTCCTACAATAATCGAAGTTCTGAACCCGGCACATCCATTACATCGTCTGTAT |
| | | GGTGAACATGTATTTCGGGCGTTAAAACGGTATCTTCCCTTGGTTACGATTTTCGAAGTTCCATCTATTAACACT |
| | | GAAGATATAATCAAGGACACTGATGACATTTTGCCGGTGCTTTTTCAGGACCTGTACGAATTCATTACCTTGCTG |
| | | TTTTACGAACTGGTAAAGTTTCGGGACGCTTACACCCATGCTCTGGCTCTGGACGAACAGGGAAACATTGTGCC |
| | | CCGCAAAATAGATCTCCATCCTAATCTTAGACATTATTTTGAACATCTGTTTTACAAAGCGCCAGATTATACTTT |
| | | ACGGCGTTTTAGATCGACTCAAGAAAAAATTGCCCACGGGTTTCATGAAAAAGACTTTGCTCTTCTTAAAGCAC |
| | | ATAATCTGTATCAGGACCCGCATAGTAACGAATTAACCACTCAGGGCTTATTTTTTTTCACCACTCTCTTCTTGGA |
| | | ACAGAAATATGCCTACCAGTTCCTCAAACGGTTCAATGGCTTCAAAAATGAAACAATCCAACCGTTTAAAGCCA |
| | | CCTTACGTGTTTTTACTGCGTTCACAGTGAAACTGCCGAATGATAAACTTGCATCAGATCACCCTGGGCAATCTT |
| | | TCTTACTGGACATGCTGAACGAGCTCAACAAAATTCCGCAGAGTTTATTCAAACACCTGACACCGAAGGATCAG |
| | | GAGATGTTTGAGAGAATAAACGATGAGGCCCTGACCAATATTCTGAAAAACACAGATGAAGCATCCTTCAAAG |
| | | ATGTTGATGACCTTTTAGGTAAAATGGCCCCGCTGATTCGGCGCGAGGATCGTTTTCCATATTTCGCACTGTGGT |
| | | TCCTCGAACAAACAAAAGCCTTCAATCACATCAGATTTCAAATAAGCATCGGTAAAATTGCATACTCCCCGTAT |
| | | GACAAAACCATTATTGGAGAAACGTCACCTCGGATAATAAAGTTTGATATCCATGGATTTGGTTCTCTGGCAGA |
| | | ATTTTCGGATGAGGATACGTTATTGACCACGTTAAAACGTCAGCTTCCCGCAGGATCTCCACATGCGAGCCTGAC |
| | | CTTTAGTCAGTATGCTCCCCACTACCACATTAAAAATAACAAGATAGGCATATTGGTGGATTATCCGTCCGGAG |
| | | ATAGACCACTCTCGAATATCACCCAGCAGCCCGACGGCAAGATCAAAGTTAACCATATTAAACCGACCGCCTTT |
| | | ATTAGCATCAACGAGCTGCCAAAACTTGTAGTTCTGGCCCTGTTAGGCACAAACAAAGACAACAAACCGATTGG |
| | | CAATGATATTGAACGCCTCATACGCAGCTTTATCACTAAGTTTAAAAATCAGCTTTTTCACCTTGCGCCGTTAAC |
| | | CAAGATCAAAAATGGTTTGACATTTTCACCGGAAACCTTCACAAGACGCATGGATAAAGACAAAATGGTAAAA |
| | | GGTCGTATTCCTAAAAGTGAACTCGCCAAACGTGAAGAATACAAGATCTATCTGGAAAAACGAAAAGCAGTTCT |
| | | GCAGGGTACCACAGACTTGAAGTTGAACGTGAATCAACTGCCTACTAAGATCCAGAATTACCTTCTTAACATCG |
| | | ATGAAGTGTCCGATGAAAAGTCGATTCATCTTCGTATCAAAACAATGATTAAAGAGGTCGAATACCGATTAAAG |
| | | AAAATTGATCGTAATAAGCTGCCAAAAGTGGGAGATATCGCGACGTATATTGCGCGAGATATGCTGGATATGGT |
| | | GGCTGATGTCCGTACAAAAAGTCGCATCACTTCTGCATATTACTCGCAAATACAGAGAGCGCTGGCGTATTTTGC |
| | | TCTGGACAAGACTCGGCTCATTGATCTGTGCGACGAGCTGGGGCTGTTCAACCATCAAACTGGCCACGTTTTTCT |
| | | TATACGGGAGATTTTTTTGAACAATCATCATATATTAGGGGTCTACCGTGAATATCTTACCAAGAAACAGAACTG |
| | | GATCGACCAAAATCTGATTGTTAAGATCCAGGAGCGAGGTAAACGTCTGACGAAGTATATCCTGCCGACTAATC |
| | | GCCCTTTGCCCTATAAGATGAAGCAATCAATTAATAAAGGCTTCGAATTCAATGCATGGCTGCAGCATAAAAGT |
| | | ACGATGCCAATCGATCTTCCTACGACGCTCTTTGATGAGTCTCTCACCAGCTTACTGAAAAAGACGATTGCTCAG |
| | | AAATTAGGGATAGCGTCAAAAAGTGAAGATAAGTTTGGGCGCCTGTTAGATCGCCTGTTGAAACAGGACACCCA |
| | | GCCATTTTACAAATTTGAACGTATCTATCGCAAACCTTTCTCACAAGAGGAACCGGAGCCTTTCAATATCTTAAA |
| | | ATTAACGCACACCGCACTGAAACAAAGATTTGGCAAACCGGCTGAAGTGAATGAGAAAATTATTCGTCACCATC |
| | | AGCGTAAGGATCAAATTGTACGCTTAATGTGTGAACACTTGTTTAAAAATAACTCTGAGTTGGCCTTCGAAGGC |
| | | GAGTTTCTGCTTGAGAATCTCCATCCTGCGAGCCGTAATAATCCGTTAGATGCGCCCATGGTCTTTGAGCAGACG |
| | | ATTCCGCATACGCACAAAAAAATCGTGGCCCAGGACTCCAATAAGTGGATTCAAGAGGTCAAAAAAACTAATA |
| | | ACCGTAACAAGGCTTGGTATCGATGGACATTAAAAGACTTTGGCACGTTTCGGAAAATTATACATGATCGTCGC |
| | | TTACCCGCCTTGGTCAATAGCATAACCGGTGATGAAATAGATTTTGATCTGGTGCAGTTCGAGCTGGCCGCCTAT |
| | | GATCGGGCACGTGAGACCATATTTGATCAGCTGTTTCTGTTGGAACAGTTCATTGCGGATAAGGACATGTCGTAT |
| | | ATCAAGCAGCATAAAGCGATAGAGCGCATGCAAGACGGAAAAGGCTTTGATGAAGTTCAATTCAACACCTATG |
| | | CAGACTGGTTAAGCGATAAAGGATTATTGACACCAGATGAGATTAATGGTCTGAAAGAAATTCGAAATCGCTTC |
| | | TCTCACAACCAATGGCCTGGTATCTCCAAACTTGGTATTCAGCCAATAAGCCAGGAACAGCAGTCAGCTTTTGTA |
| | | ACCCATCTGAATATTAAAGGTTGGGTTAATGAACATCATACGCCCCTCGCCATGTTTATGGAAAAACTGGTAATT |
| | | AACTGGGTGTCAAAAGCGATCACATAA |
| 309 | JW13132 | ATGAAAATCAAAAACGAAAATACCGACAAGAAAACCGAGTTATACTCTATAGATAAGTATAAGGGACGGGATA |
| | | AGTGGTGTTTTGCAATTGTGCTGAACAAAGCTCAAACAAATCTTACGGAAAACCCTGATCTGTTTGAACAAACA |
| | | ATTACGAAGTATGACCGCATTCGTAAAGAAGGTTGGTTTGATGAAGAGACGAAGAAATTAATCTACATTCAGGA |
| | | AAATGAGCATAAAATCAAAGGCGAAATCAAGACATTAGCGCGCGAAGTGCTCAAAAACCTTCGGAATTATTTC |
| | | AGCCACCATTTTTACAAACAGGATTGCTTAATTTTTCCTAAAGATAATATAGTGCGAATTATCATGGGTCGTGCG |
| | | TATGAGCGCTCTGAATACGAAATTAAGAAAAACCTTAAAGAAGACATCAGTATTGAGCTGCCGGCTCTGTTCGA |
| | | GCCGGAAGGTAAAATTACCACCGCTGGAATTGTTTTCTTTACCTCTTTTTTCGTGGAGCGCCGGTTTCTGCATCGC |
| | | TTAATGGGCTCAGTTCAGGGGTTTACAAAAACAGAAGGCGAATATAAGATAACACGTGATGTCTTTGCGAAATA |
| | | TTGTCTGCGTGATTCTTATTCTGTTAAAGCGCAGGATAATGATGCGGTTATGTTTAGAGATATTATAGGTTATCTT |
| | | AGTAGAGTTCCAACAGAATCCTTTCAACATATTAAAAATCCAAAAAAACAGAATGAGAGCCAGCTGTCCGAAC |
| | | GAAAGACGGATAAGTTCATTAGTTTTGCCTTGAAATATTTGAAAGACTATGGTTTTGAGGATCTTAAGGGCCATT |
| | | ATACCGCATTCTTTGCTCGCTCTGAAATCAAAAAAGAAAAAGAGGATATTGAGATCAAAGATGACAAAAAACA |
| | | CAAACCCCACCGGATGAAATCGAAAATAGAAATTCACTTCGATAAAACCAAAGAGGATCGTTTCTACATAGAAC |
| | | GAAACAATGTCATTTTAAAGATTCAACGTAAAGGTGGACGTGCAAATATTCTGCGTATGGGAATCTATGAACTT |
| | | AAGTACTTGGTGTTGTTATGCCTGAGTGGTAAAGCCAGAGAAGCCATTAATCGCATCGATGATTACCTGAATGA |
| | | TCTTCGGAATAAAATCCCGCATATTGAGAACATGAATAAAGAGGGGATCGGTGAACAGATTCGTTCGCTGCCGG |
| | | GTTTTGTTAGATCCCAATTAGGTTTCGTTCAAATAGACGATGAAAAAAAGAAAGAAAACCGTTTGGACTATGTA |
| | | GAGAAAAAGTGGGAAAAAAAACGTGCCGAAAGTAAAGAACTGAAACTGAACCGCAAAGGCCGGGATATTCTG |
| | | CGCTATATTAACGAGCGTTGTAAAAAACCTCTTACTATCGATAGATATAATCGCATCCTGGAGTTGCTGGTCGAA |
| | | AAAAACATTGAAGGGTTTTATCATGAGTTAGAAGAGCTCCGGAAAACTGGCAGAATCGAAAAGAATATAACTC |
| | | AGGCACTGGTAGGTGAAAAGAATATCAATGCGCTGCATATTAAGATATGCAAACTGGTGCAGGATGAGTTAAA |
| | | ATCATTAGAAAAAGAAGATCTGAAAAAGTACATTGGGTTAACGCCAAAAGAGGAGAAAGTAGTTAGCTTTGAA |
| | | GAGAAATTAGGTCGCATTCTTGACAAGCCCGTAATCTACAAGGGCTTTTTACGTTATCAGTTTTTTAAAAACGAC |
| | | AAAAAGTCATTCGCACGACTCGTAGAAGAAATCATTAAAGAAAAAACGGGCGGATTAGACGTTCCAATTGAAA |
| | | CTGAGTATTATTCCATATCAACACTGGGGCGCTTTGATAAAGCAAACAAAACTCTCTATGAAACTCTGGCTATGG |
| | | ACCGTCTGTGCATGATGATGGCCAGACGCTATTTTCTGTCGCTGAACAAAATCCTGGCGAAACGTGCACAAAAT |
| | | ATTGAATGGAAGAAAGAATCAGGCAAAGAATTCATAGTCTTCAAGTTTAATATGCCTCAGGATACTGGAAAAAG |
| | | TATAAGTATTCGATTTTCACCGAAAGATTATACCAAACTCTACGTGAAAAATGACTCAGAATTTCTTGCTCGCCT |
| | | TTGTCAATACTTTTTTCCCAATGAGAAAGCCATTGACTACCATAAGCTCTATAGCCATGGGATCAATAAGTATAC |
| | | CAATTTCCAGAAGGAAGGCATTGAGGCCATTTTGGAATTGGAAGAAAAAATAATTAAGAAACGGAAGATCAAT |
| | | TCGCCGGAGAATTATTTGAGCTTCGAAGAGATTTTGAACCAGTCTATCTACAACGACGAGGAAAAAAACACCCT |
| | | GATCCAGATACGTCACTCCCTTCTCCACTATCAGATCCTCTTCAGCAAAAACGACCTGACGAAGTTCTACAATGT |
| | | CATGAAACGCGAGGGGATTGAAAAAATATGGAGCCTGGTGATATAA |
| 310 | JW13133 | ATGGAAAATCTCTCAAAAGAGCAAATTGTTGAAACTCAGGAGCAGAATAAGCGCACCTGGGGTAATGATCCTC |
| | | AGTATTTTGGAGCATATCTTAATATGGCTCGTCTGAACATCTACAATGTTTCTAATCATCTGGCCGAAAAATTTG |
| | | GTATCACAGCAATTGAGAATGAAGAGCAAATCAGCAACTCATTTCTTGGGGACAAAGAGAACAAAGTGTTCAA |
| | | AAACAAGCAGAATCATATTTTTTCCAATCTGATACGCTATCTTCCAATCGTTAAGGTATTTGATTTCGAAAAACT |
| | | GCCGGAAGAGGAGAAGATTAGCTTGAAAGATAACTTCGGAAAAGACTTCAAAGAGCTTTCGGATACATTAAAA |
| | | ATAATTTTTAGCGAGATCAATGAGTTCAGAAACGACTACAGCCATTATTTTTCTACCGAAAAAAAGAATAAACG |
| | | CAAAACCAAGGTCTCTGATAAACTCAAAGATTTTCTCAACATTAATTATACGAGAGCAATTCAGTATACGAAGA |
| | | ACCGTTTCATTAATGTGTTCTCAGATAATGATTTTTGTTTAGCCGAAGAAGCAAAACTGGTCGAAAATGATAACA |
| | | CCATTACTCAAGATGGTATCGTATTCTTTACGAGTATGTTTTTAGACCGTGAAAATGCATTCCAGTTCATTTCTAA |
| | | AATTAAAGGTTTAAAGGGAACTCAGACCAAAGCGTTTAAAGCTAAACGTGAAGTTCTGATGGCCTTTTGCATTA |
| | | ATCTTCCACATGACAAGTTTGTTAGCGAAAATACAGAACAAGCCTTTAGCCTGGAGTTAATAAACGAATTAAAC |
| | | AAATGCCCGAAAACGTTATACAATGTGATTTCTGAAAATGAAAAACAACACTTTTTACCGAAACTCGAGGATGA |
| | | ACAGATCGATAATGTGCTGGAAAACAGTGTTCCCTACACGATTGACGACTATGAGAACTATATCGAAAATATCA |
| | | CAAAAAAAGTGCGAAATAAGAATCGCTTTTCGTATTTTGCACTGAAATTCATAGATCAGAAAGAGCTTTTCAAA |
| | | AATTGGAAGTTTCAGATTGATCTGGGTAAAGTGGTCTTAGATGAGTATACGAAAACTCTGCAAGGCAAAGACGA |
| | | AACAAGAACTGTAGTTGAAAATGCCAAAGCGTTTGGGAAACTTCAGCACCTTAATGACGAAGAGAATGTCTTAA |
| | | ACAAAATTAATAAAAGCGAGTCGAAGACTCATTTTGAGCAATTTGCGCCTCACTACAATTTTGATAATAATAAA |
| | | ATAGGTATTTCCCGGAAAGAGGATTCAGCGATTTTTATCAGTCGGAATACCGACAAAAAAGTTAAGTTTAATCT |
| | | GAAACAACCTCTTCCAGAAGCTTTTCTGTCAATCCACGAATTACCCAAAATTATTCTCCTCGAGTATCTGGAAAA |
| | | TGGTAAAACCGAACAACTGATTAATGATTTTATCGAGATCAACAATTCCAAAATCCTGAACAAGAAGTTCATCG |
| | | AGAGCATTAAGAGTAAGCTGACAGATTTTAATACATTCAATAAGCGCTCACAGGGCAGAAAAGAGAAATCAGC |
| | | CTATAAGAAGGAAGCTCTCAAATATTTGCTGGATCGTAAAGAAAAGCTGAACCAGATTTTAGCTGAGTACAATC |
| | | TTAATGTAAAACAAATACCGACGCGGATTCTGGATTATTGGTTAAACGTGGCTGATGTCCAGGAAAAAACCGCG |
| | | ATATCTGACCGCATTAAACTGATGAAACGGGATTGTATGGATCGTATCAAAGCTATGAAAAAAGGAAAAGCAC |
| | | CTAAAATAGGTGAAATGGCAACCTTTATTGCAAAAGATATCGTGGACATGATTATTGGCGAAGAAAAGAAGAA |
| | | AAAGATTACCTCTTTTTACTATGACAAAATGCAGGAATGTCTTGCGCTTTTCGCGGACAAAGAAAAAAAGGAAT |
| | | TGTTCATTCATATATGCAACAATGAGTTGAAACTGAATGAGTCAGATGGCCACCCGTTTTTGAAAAACATCATTC |
| | | TGTATGATTACCGTTATACCTCGGATCTTTATCGTCGATATCTGGAAGAAAAAGCCGATAACCGGAATAATCTCA |
| | | ATAAAAACGATACATCGTGGCTGGCCAAAAATTTCTATTTTATCGAAAAAGTGGAAGTATTCGATAAACGACTG |
| | | AACCGCATGGAATTCAAGCCGCAGACAGTAGTCAAAATCAAAGACAAAAGTAAGATACCGTTTACCCTGCGTC |
| | | AGTTGGAAAAAGAAAAAAGTAGTTTCGATACGTGGTTCGAAAATATAACGAAAGGCAAAGAAACAACTGACAA |
| | | AAAAAAGCCTGTCGATCTTCCAACCAACCTGTTTGATAACTATCTGAAGGAATTATTGATTAAGGATCTGTCCAA |
| | | GAACAATATTAAGTTTAACCCAAAGACTAATTACAACGAGTTATTCAAGTTGTGGTGGAAAGAATGCAGAAAAG |
| | | ATAAAACTCAGTCCTTTTACAATGCGGAGCGTGAATACTCCTTTGAGGACAAAAAACTGAACTTTAAGATAAAC |
| | | ACGAAGTCCAAGTTTGAAGATTATATCAACCCCGAATTTGCTGAAAAAGTTTTTAAACTGAAATCTTCTCGCCGT |
| | | GAAAAAGAAAATTCTACCGGGCGCAAACTTCCGCCTCTGATGATTAAGGATTTGGAAAAATCAATGGCGAACA |
| | | AAATTGGGAGTATAGAAAAAGACATTCGAATTTTACAGGAAGATGATTGTATGATGTTGCTGATGTTCGAGAAA |
| | | CTCGTTGATAATAATCTGGAACTGAAACTGGAGAACGTGGACACATTGATGAATGAGAGCATTGAAATCAAACA |
| | | AAGCATATTGGGAAAACTGAGTTTTGACGACTTTGGGGACAACGTAAACAAAAATGATCGTATTATCATCAGCA |
| | | AAAATATTACTGAGACCCGGAAACGTAAAGAGTATTCAGTTTTAAAAAAATATATTTTCGATCGCCGCTTACCC |
| | | GAGTTATTTGAATATTTCGAAACGGAAGACATTACCATGGAGAAGATAAAGTTAGAACTGGGTACTTATAACAA |
| | | AGCAAAGGATATCGTGTTTGACTTGATATTCGACTTGGAAAGTGCCATCATCAAAAAAAACGAAAACCGGATAA |
| | | AAGAGCTCAACAAAGATAAAAACGGCAACATCCAGACGGGCAACATTCAACATAAACCGTACCTGTTCTGGCT |
| | | GCTCGAAAAAAATCTTATAACTGAACAGGAGTTCACCTTTCTTAATATGGTTAGAAATACATTTTCGCATAATCA |
| | | GTTTCCACAAAAAAAGACAATGGAACTTTTTATCAAGAATTGGAATAAGAACGAATACGCCAAGCAGATACTG |
| | | ACAGTCTACACCGAAAAGATTAACAATCTTATCGAGAAAGTTATTGATATATGA |
| 311 | JW13134 | ATGGCTGTGAATTACAGCCTCAGAGAAAAATGGTATCGTGGAGTTAATAAGTGTTGTTTCACAGTAGCACTCAA |
| | | CATTGCAGTAGATAACTGCAAAAGTAAGGGGTGCGAAACCTTACTGAAAGAGGCGGAACATTCTAAAGGTGGC |
| | | ATAACCGATGAACAGATTCAGCAGAGTTATACGGAAGTTGAGAAACGCCTTAACGATATAAGAAACTATTTTAG |
| | | CCACTTCTATCACGGGGATGAATGCTTAATATTCAAAAAAGACGACATCGTCAAGCGCTTCATGGAATCTGTCTT |
| | | TGCTACTGCGGTGAGTAATGTAGTGGGCGGTACTAAAGAATCAGATTATAAAGGTGTTGTCCCGCCCCTGTTTG |
| | | AACAATCCAATGAAGATTATATGATCACTGCGGCCGGCGTGATCTTTTTAGCCTCGTTCTTTTGTCATCGCTCAA |
| | | ACGTTTATCGTATGCTTGGCGCTGTTAAAGGTTTTAAACATACCGGGAAAGAAGAACTGTCTGATGGTCAGAAA |
| | | AGAGATCATGGTTTTACTCATCGTCTGTTGGCCCACTATAGCCTGCGTGACTCTTATTCAGTTAAAATAGAAGAG |
| | | ACCAAAAGTTTTCGCGACCTTCTGGGTTATCTTAGTCGAGTGCCTCAACAAGCTGTGGATTGGTTAAACGAGCGT |
| | | AACGAGCTGTCGGAAGATGAGAAGAAAGAGTTCCTGAACCAAAAAAGCAGCGAAGAGGAGTCGCCGGAACAG |
| | | CCTGAACCGGAAAATGCCGAATGGCGTACTGAAAAGACAAGTCGCCGCTCATTACGTAAAACGGAAAAGTTCA |
| | | TTTTATTCGCCGCTAAGTTCATTGAAGATAGAGCTGAAAAAGAAAAACAAGATGTTACCTTTGCGCGTTACCAG |
| | | AAAACGGTAACAAAAGAAGAGAATAAAAATCAAGACGGAAAACAAGCCCGCGTCGTGCGATTGAAATATGAG |
| | | GAAGATAAAAAAGATGATGAAAAACCGCGCGAACATTTCAACCTTGAGTGGATGTATTATATTCGCAATGAACA |
| | | CGCAATCATCCAGATCAAACCAAAAGATAAAGAGGCCGTCGCAGCACGTATATCTGAAAATGAATTAAAGTAC |
| | | TTGGTGCTGCTTATCTTCGAGGGAAAAGGGGGAGATGCTTTTAACAAGCTTAGCGACTATATTTTTCGGATGACA |
| | | CAGAAAATCAAGAGTGGCCAAATCAATCCCAATGAAGCGCGGCTCCCTAGCTTTCTGAAAAACCCAGTCAAAA |
| | | ATATTACGGACAAAATGGTGCGAAATCGTCTCGACTATATTCGAGGGCAGATCAAAGATGTGTTGGAAAAGATT |
| | | AATATGGAAGAGCCGCAGAACAACAAGTGGCTGATATACAAAGGCAAGAAAATCAGCCTCGTGTTGAAGTTCA |
| | | TCTCGGATGGGATTTCAGATATTAAAAAGCGTCCAAACGTGAAAGAGTATGATACCCTGCGGGACACATTACAG |
| | | AAACTGGACTTTAATCGTTTCTACGAGCGGCTGAAGTCCTACGTTTCCGACGGTCGTCTGGCTGCGGCGCTGTAC |
| | | GATAAAATCAAAGGGATTGATGATATTTCTGAGCTGTGCAAGAAAGTCTGTGAACTCATGTTTGCACGCCTGGC |
| | | CGAGTTAGAAAAAAAAGGTGGATTTGAATTGTATCGCTACATCGGAATGGAAGTTCAGGAAAAGGATGAGAAA |
| | | TACGATGAATGGAATTCCCCACAGAAGAAAGCCGAAAGATTTCTGGAGAGTCAGTTTTCAATAGGCGAGAATTT |
| | | TCTTCGTGAGTCCTTTTATTCGGAGTACTGCCAGAAACAAGAGTGCATAGATAAAGAGATTTCCCTCAATACAA |
| | | GCGTAAAGAATCGGAAGAGTTTGGTATACATTGTGAAGGAAAAGTTGAAAGACATTATGCCTCTGCATAATGAC |
| | | CGTTGGTATCTTATCGATCGAAATCCGAAAGATTTTGAACGAAAGGACTCAAAAGTTATCAAAGGCCTGTGTAA |
| | | TACGTATGTACAGGATGTTCTTTGTATGAAAATGGCGAGATGGTATTATGGTCAATTAAACCCAGCGCTGAAAA |
| | | ATAACATTAAGTGGGATGAAACCGGTCAAGGACATGGTTATGACCGTTACAAACTTTCCTATCGTACCAACTTC |
| | | GGAATTACTATAGAGTTCAAACTGGCAGACTTCACACGGTTAGACATTATAGAGAAGTCAGATATGATAGAGAA |
| | | CATCTGCCGCAGCTTTATTAAGCCGAATCGCACGATTTCTTGGTACGATTTTAAACAGGATGGTGTTGAAGAATA |
| | | TAGAAAACGTCAATATAAAGCAGTACGTGCAGTTTTTGCATTTGAAGAGTCGCTGATTATTCCGGGCCGGGACT |
| | | GGCTGTCTCAGGGCTTTGTTCCCTTTATTAAGAATGAAGAATACGTCAAAAAAGGCTTCAGCCTGTTCGTATTAG |
| | | ATGAAGCTGTTCGGCAGTTAAAAATTAAAGGTTCTGACAAAGACGCCATGCGCCAGGTGCGGAATGATTTTTTT |
| | | CACGAACAGTTTCAGGCGAAAGATGAACAGTGGAAAGTCTTTGAAGGCTACTTGTCATGTTTTATGATTGATCG |
| | | CCCTAAAGGTGAAAAAAACAAGAAGAGATATAATGGGAATAAAAAATAA |
| 312 | JW13135 | ATGGAAGAAAAAGAGAAAATTTCTATCCAGACTATTCAGGATGCTCCGTATTATTTTTCTCATTATGCTAATATG |
| | | GCGCGACACAATGCTTTCCTGATTGTGAATGAAATTAATGAAAAGGTCTTCCAACGTTCAGCAACAAAAGAGGA |
| | | TGAACTGCTGCGTGAAAGCTTTCTGGTTAAACCGCCTAGACAGCAGGCTGATATTATTGGAAAAATAGACTCAT |
| | | TATTACTTCACCATTTTCCATTTCTCGCCTATTATGAGAATAAAAGTGCCGGGTCGGCCCGTGTCGAGTTAAAAA |
| | | AATACATGGACATCTTAAACCAATTTCGCAATGAGGTGACACACTACAAGCATCCAGTTGAACCAAAGTGTATC |
| | | GTCAATCTTCAGGAAGTGTTTTCTTCAGCCGTGGAAAAAGTCCAGGAACGCATGATTGCCTTTTCTGAAAAAGAT |
| | | TTTGAGCATCTTAAACCGAGCTCAAGTGACTATCGTCATTATATCTTAACGGAAGCCGGTATCAATACCTTGACT |
| | | GACACGGGTCTGTATTTTTTTCTGTGCTTATTCCTGGAGAAGAAGTACGCGTATAAGTTCTTGGCCGGAATTAAG |
| | | GGTTTTAAAAATAACACAGACCATACACACAAAGTCGCATTGGAAGTTTTTACGCAATTTTGTTGTCGTCTGCCA |
| | | TATTCTAAAAGTGCATCCTCTGATATTGCACTGGATATGCTGAACGAACTGAACCGATGCCCTAAAGCGCTGTAT |
| | | CAGGTAATAGATAATGAGCAGAAAGAGCTCTTCACCGTCAAAAAAGAGGATATTGTGGAAACCGAACAGGGTA |
| | | CCGAGGATGTGCCGGAACCCGTTATGATTCGTTATGATGATCGCTTTCCGTATTTTGCTTTACGTTATTTTGACGA |
| | | ATCAGAAGCTTTAACTGATATCAGCTTTCAGCTGTATCTGGGGAGAAAGCATAAAAAAGAACTGCACCGCAAAA |
| | | AATTAAACGGCGAAATGCGAACCCATAATCTTTTGAAAGAAATGCATGTGTTTGGCAAACTGGCGGATTATCAA |
| | | ACAGAAGAGATGACCAAAAGTTTTATTGAATCAAAAGCAGTTGAGCACTATGCACCGGGTTATAGAATTGTAAA |
| | | CAATCGCATTGGGGTTTTACTGAAAGCACCCGAAAAGTGTTCCCTGACGAGTAGTATTCCGCAAAAAACAAATA |
| | | CTGCGCCGTCTGCAATACTGTCAACCCATGAGTTAGCAGCCTTGTATCTTTATAATAGACTGTACAAACAAGGGT |
| | | CCGCTGACAATTCCCCAGCGTCTATCATTAAGAAGTATATCGAAAACTTCAACCGCTTTATCAAAGACCTTCAGT |
| | | CGGGAAAAAGTCAACCTGTAGAGAGCGGGGGATTTCGTCGCATGCGGAATAGTAATAGAGATGAAGAAGAGCG |
| | | TCTGAAATTACAGAAAGCTAAACAGGAGCTGCAGAAACAGTTGGATGTTTACGAACTGAAAGTGTCTTACCTTC |
| | | CGGATGCATGCTTAGAATATCTGCTCTCCTATAAGAAAGATAACAAGGAATTCATGTTAAAAGATACGTTCCGG |
| | | AAAATGTGGAAGAATGCAGATGAAGGTTTGGAGAAAATTCGTAAAAAAGAGGAGCAAAAGAAAGAAGTGAAA |
| | | CAAGAAAAACTCGCGCAGGAACTGGCGCAAGATTTTGTGTATCTGACACCGCCACATAAAGAATTGTCAGAAA |
| | | ATGGCACGGAAAAATCCGTAGACAAAAAAATTAACAACATGGAATTTGACGTGCTCAAAAACATGCTGACCTA |
| | | TTTCCCGCTGTACAAGAATCGTTTAAAACCATATCTGAACATACTGAAAGATACTGAGAACAAGTGGCAACACC |
| | | CCTTCCTGCATCTTGTCCTGATCTACGATTTCGATTCCTGCAATTCATTGAAGGATTTCTATGTGCGTTACTACGA |
| | | ATACAAGAAAATATGGATTAACAAGTTCATCCAACAAGATAAACGGAAGCGGTGGAATCTTGTTCCAAAACAG |
| | | AATGTCAATGATCTCATTAAGAAATATGACTACTTTCTTAAGACCACGGTGAAGTTTAAGAGTGCGAAGGAGAA |
| | | AGAGTATTCTAAAGATGCGATATTTCTGCCTAAGGGCTTATTTGATGCCCATATCGCTGAGGCTATGAAAAAAC |
| | | AGAACTATGAAGTTAAAGAAACCGACAACGCTGCCTATTTTTTGAAAGTTTACTTCGACGGTAAAAGCCAGAAT |
| | | TACTATAGTCTCCCCCGCTACTATGACGCGTCAGCAGTTGGCTGGCTGACCCATGAAGCGCGGGATATTCTCAAA |
| | | CGAAAGATAGAAGAGACTTGGGAAACCAAAAATGACGAGGAGTTCCGCAATAAACTCAAAAAGTTAAGCAAA |
| | | GATATTCGGGAGTCGGAAACAGAACTGTTGCATCAGCAGACGAATGATCGCACTTTATTTCTTATGGTTAATGAT |
| | | TTGCTCAGCGGCAACACTAACAACTACAAAGACATTGTAGATCTTGGCTACAATAACAAGAGTGAAAACTCGAT |
| | | TTTGAACAAGCTGTATCCTATCAAAGAGACCATACACGACAAGATTGTCACTGCAGAACTTCCTATTAAACGCT |
| | | ATGGTGAATTCAAACGCTTTATGAAAGATAGACGATTACCCGATCTGCTTGGTTATTATGGAGAAAAAGAAATA |
| | | CCTCTGAAAGTACTGGAGACGGAAATGGAAACCTATGACCGTATGCGGAAAGATGCCATAGAGCAGATCTACA |
| | | ATTTCGAGAAATTGATCTATGATAACTTCGAGTCGGAACTCAAAAAAAGCGTAGACCCGTCGGCCTTATACATT |
| | | GATCATAATCACTACCTTAACTTTGTAAAGCAAAAGTTCAAATTGGAAACTGTTATCACTGAGATGCAGAGCGA |
| | | ATCTTTTAGAGAATTACGCAATAAAATAGAACACAATCAGTTTCCGCCTCGTGAAGACTGGATAATCGACAAAA |
| | | TTAGCCAGGATACGACAACGACAATAATCACAGAGAAAATCATGAATCTTATCTCCACCGAGTACAAAAAGATT |
| | | TGGCGTGAAGTTGAATCAACAACCAGCATTAGCGATATGGAATAA |
| 313 | JW13136 | ATGAAATCTCCATCGAAAAATCCGCGGTCCGAACAGGCGGTGGATTATGACTTCTTTGTCGGTGGTCGTTCCAA |
| | | AAGTGCAGATCTTACCATCTACTACAATGTCGCCTTTGATTCTGTTGTTAAGATCAAAAAGCAGATATCAAACTG |
| | | GCCTGACAGCCGGCAATATGCGTTTTTAGCTGAGTATCTGTGGAAAGCGCATTACCTTCAAAAAAATAGTAATG |
| | | GCTATGAGGTGCAGCCAATCGATGAATGGATTATACAAGCCCTGATTGCTAAAGTTGAGGCGATCCGTAACTTC |
| | | CATTCTCACATTTGGCACGATCATGAAGTGCTCATCTTCGAAAAAGAACTGAAAGACTTTGTGGAGCAGAAATA |
| | | CGATGAAGCGAAAGCCGCACTCAGTGCCGAATTTCCGGGAGCAATGCTCGAGTACGAGGATCTTGCGCGCACCG |
| | | GTCGCATGAAGAAATTTTCACTTTTCAAACGTGTTCCTGAACGGAAAAAAGACTTTATTACAGTTGAGGGCCGC |
| | | ATCTTTTTCCTGTCCTTCTTTCTGTCTACCGGGGAAATGAACCAGTTACTGCAACAGCGACGCGGCTTTAAACGT |
| | | AGCGATATGCCACTTTTCAAAATGAAACGCCTGTTGTACACCTTTTACTGTCACCGAGATGGCGCCTCGATGTTG |
| | | GATTTCAATCATAAAGAACAATTCATAGATTCTATGGATGAGCAGCAGAAACGTGACATCTTTAAGGCCCGGAC |
| | | AGCGTATAAACTGATTAGTTATCTGATTGATTATCCCTGGTACTGGGGTTCCAGAGACTCAATGCCACTGTTTGA |
| | | TGAAAAAGGAGAGATTATTCGGAATGTTGGCCAACTGAAAGATTATCTTGAGAAAAATCCGCACCTGCTCCCGG |
| | | AGCTTAACTTTCATCTTTTAGATCGAAAGATCCCTGCAACTGGCAAGTTTAATACTCGCAAGGAAAAAGAGAAT |
| | | TTTGCAAAACTGCAGGATGATAGATATAGATTAGGAACAATAGCTTTTAACTATCTTCCTATACCCAATTATACC |
| | | TTTCACATCGACTTTGAAACGTTACATCGATTGGTATTGCTGCAGATTCTGCATCACGGTTCTAAAAAAGAGGTT |
| | | TCACCGTATCAGGTGTTAGAATTGAACTTACGGAGCATAGTGGAGAACCGTGTAAGTCTGTACAACATTCTGTG |
| | | TAAACAGGATAATGAGAGAACGGGGGAAGATCTTGACTTTCTGTTAGACCGCAAAAATCAGAGACTGAGAGGT |
| | | GGCCGCAAGCTGACCGAATTTGGCATCAGCTATTTTGAAAGTATTGAAAAAGGAAGCGGTGCTAAAATTCGTGA |
| | | TAGTATCCAGCTGGCTAACTATATGCGTTCGATGGATACAGCATGGTTAGAATTACCTTATGAAGGAGCCCATA |
| | | AAGTTGAAATTCAACCCCTGGATCCGGAACCGATTATGGTATACCAACAGGATTTTTTAGAAGGCACCACGCAG |
| | | AAATTTCGCGCCGGAAATCGTTTTATGTTTTACGTATCTCGGTATCTTATGGATTTCGCAGGAGACAATTGGTAT |
| | | TGGGGAATGGAACGTTTTGAAATGCGTAAAAAAGATCAGAACGCCCGAAGCGAAAGCTTGATTCGCACTAAAG |
| | | AGTACCTTAGAGCATCCGATATACCTCTGAACGGCGATTACCGCTTAACTCTGGATAACGATCATATTTTCTTAG |
| | | CGATGCCGAAGAATGGTCCGGGCAAAAAGAATCATGAAAAATTTTACCAATTTGCATTAGGTCCGCGTGCCATG |
| | | CGTTATCTTGGTGCCTATATCATTAACAATCGTGCTGACTTTGTTAGCAAGGCGAAAGGATTTTTGGATACATTA |
| | | GTTCGCGATCTTGACCGTCTGCACAAAAATGAATACTGGAATATGGAACATGGGTACGAATTATTGGAATCCCC |
| | | CTTCGTTTCAGGGTTTCTGAAGAACGTTCCCACGAATATCGAGAAACTGTTAACGCTCGTTAAGACGCGTATCGA |
| | | GTATATCAAGCAGGAGTGGCAGCAAGCTCTGGAAAATAAGCCGTATCTGAGTCGCGCGCAAAAAAACAAACTG |
| | | ATAATGGATGCATATCGTCTGTTACAGTGGCCGAAAGGCAACGATGGCAATCCACGTTTTTTTCGCGCCCATGA |
| | | ATACAATCAGATGTCGGTATGCCATTATTCTCTCCATTTGAAGGACAACGAGCAGGATAAAGAGTCGCGGAAAC |
| | | AAATTAAAAAACGCTTCAAAAGTAAGTATGACCACCTGTTCGAGGACGTCTTCAAACTGAATACTCGTAAACCG |
| | | CCTATTCCTCGCTCTATTCTGTCACTGCTGGACGAGGCAGATTCACTTGACGAACTCATGAAGAAAACCTTTGAG |
| | | GAGTGTCACCAGCTCCTGGATAATCGTTATTCACAACTCAAATCGTTACCAAATCAACAGCTGAAACGAGAATT |
| | | ACCTGAGCTGTGCCGCCTGTTAGGAATCTCAGTGCCGTTAAACATCCTGCGAGAACAGGAAAAAAAGGCCCTGA |
| | | AAGAGAAGCATAATACGACTCTTTGCGTAAAGCCGTTCGATATCCATCCCATGTTAGTTGTGAAGTACTTTTTCC |
| | | CAGAACAGTATAAAGCAAGTAAAAGTGTAGAATTTGTCAACAAGAATGGTGAAAAAGTAAAGAAGCAACCAAC |
| | | CTTGACGATTTTTTCAGCGCTGCGTAAAAATAGAGCACTGAGAGAAAAACTCGTGGAAGACTTCTACGACGAAC |
| | | AGAATAGCGCTGTGCTCTATCCCGACGATGACACAAAAAAACAGAAAGAGGTGCTGACAGGTTTGATGAACAC |
| | | AACTTGTACTGAAGACATACTGTTGTGGTGGATGGCTAATGAATATCTTGTCAATAATCCCTATACACAGGCTAT |
| | | GGGTGAGCTTTTAATGAAAAAACCACGTTCCGGTCGGCAGTATATTGGGGATCTTCATAAACTTACCATTTTACA |
| | | ACCGCTGCCAAAGGAAAGCGGGAAACGGCAAATCTACATGAGTATTTTGATGCACCAGTTGGACGACCTTATGT |
| | | TCATTGTCCAGAAATCCCGGTTAGGCAAAGCGGCGGAACATTTTGTCCAGCGTTGCTCCACCGAAAATGGGATT |
| | | TGGGCTCCGGAAATTGAAAAATTATCAACCAATCAATTGGCAGGGGCTCCTTTTCCGGATGGTACCATTAACAA |
| | | CCCTATTCCTTTTCAGCTGCTGCTCGATGAGATTGAACTGGTCCGCCGTACTGGCCTGGAATTGGGTAGATATCT |
| | | TTTGGAATTCGAAAAGAAGATACTGCAACAGGCATTGAACGGCTATGAGAGCAAAGAAGCGTTCCATAAGTGG |
| | | CTGAAGAATGAGTATGAGAAAACAGAGTATTATGGGAAGTACCCAGCCTATTTCTTCAATTTTGATAGCATACT |
| | | GAAGCTGTCTGGGACAGTGGACAACAACATAGACAAAGATCTCAACATAACGTTATCTGATTATCGCAACATCA |
| | | CGTTCCACAACGATATCCCGTCGAATGGTAGCTTTAGCTGGTTAACTCGGGAAGGTTCTTCCGTGCGTCAGTTAC |
| | | TCGGTATAGAAAAAAATTTGCATGAAAAACGTGATCGCTCAGAGTATGAAAATAATCGTGAAGAATAA |
| 314 | JW13137 | ATGGAAAAAGAACAGGGGTTATACAGTATTGATCGCTATCAGGGGGCTGGGAAGTGGTGCTTTGCGATTGGTGC |
| | | AAATCGAGCTTGGGATAACTATAACGAACGACCAAAACTTTTCTCCGAAAGCTTATTACGCTATGAAAAAGCCA |
| | | CTCGTCGTGACTGGTTTGACGAAGAGACAAGAGGACTTATCAAAAAATCTGACGTTCGTCAGCGGCTGCGTAAA |
| | | ATCCGGTGCTATTTTTCCCATTATTGCCACGACAATACCTGCTTAGGCTTCGATCCGGATGACGATCTGCGGAAA |
| | | ATTATGGAGAAAGCGTATGAGCGTGCCATATTTGAACAACGTAAACATCTTAGTACCGAGACCGACATTGAAAC |
| | | GCCAGCATTATTCGAGCCTCATGGTAGAATAACTGCCGCAGGGGTTGTATTTTTCTGTAGCTTTTTTGTGGAGCG |
| | | TAGAATCTTGAACCGTCTGATGGGTCGTATTCCGGGCTTCAAAAAAACGGAAGGTGAGTATGGGGCAACTCGGC |
| | | AGATGTTTTCTAAGTATTGCCTTAGAGACTCATATAGTATTCGCGCATCGGACTCAAATGCGGTCCTGTTTAGAG |
| | | ATATCCTGGGTTACCTGTCACGGGCACCGAGCCAATACTATCGCCACAATAAAGATCAGTGCGACAAAGATGGC |
| | | CATCCTGAGAGAAAGAAAGATAAGTTCATCAATCTTGCATTGAGATATCTTGAAAGCTTCGTACCTGCCCGTTTA |
| | | CGCAATCATACGCTCTCTGTCGGTCGTAAAGAAGTCGTTCGTATGGAGACAAATGCCGTCGCTGAAGGCGAAGG |
| | | CGAATACCGCCCGTATCCGCCCAAAGCAAAGGTGAAAGTTGTATTCACTGAAGATGATCCGGAGCGACCATACT |
| | | ATATTACTCATAATACAGTTATTCTGCAGACCGCAAAGAAGGAAGAGGACATTCACCATTGTAAAGTTGGCGTG |
| | | AACGAGCTGAAGTACCTTGTGTTACTCTGTTTGCAAGGAAAAGCCGAAAAGGCTGTCGCAGGCATTGAAGGTTA |
| | | TGTGCGCAGAATCCAGGGGCGCTTTGCCGATCACACCAATAAAGTAGCGCGCGATGATGATGAACGGCTGGTAC |
| | | GCGGCTTGCCAGAATTTGTCCGCGTGGCCTCAGGAATTGAAACACCCGACGAAGTCCGGGAGCTGAAAAGCCGT |
| | | CTCGATCACATACGGAAAAAATGGCAGACCAAGAAAGCTGAATCGGCTGAAGCGCAATTACATCGGAAGGCTC |
| | | GGGATGTTTTACGGCACATCAATTGGGAGTCACAGCGACCACTCGGAATCGAGCAATATAACCGGTTACTCGAG |
| | | CTGTTAGTTAACCGTGACCTTGAATCCTTCGCAGCCGAGATGAAGGAACTGAAAAGACGCGGATTGATATCCGA |
| | | GGAGCTCTTAAAAAGTGTTGAGGGAATTCGTAACCTGAATACACTGCATGTGAAGGTTTGTAATCTGGTCCTGA |
| | | CGCGCTTGGAACATCTGGTTGAAAACGATCCAGAGGAGTTAAAACGACACATTGGTATTGTACCGCGTGAAGAA |
| | | AAAGAAGGTCCCTCATACGAAGAAAAAGTTCGTGCCTTTGTACAGCAACCTATGATGTATCGTGGTTTCCTGCG |
| | | AAATTCCTTTTTTAAAGGGTCTGGCAAGAGTTTTGCCAAACTTGTGGAAGAAGAATTGCACAAAAAAGGTTGTC |
| | | CGGATGTGCCTCTGGGCACCGACTACTACCTTGTGCGCGATTTGGAACGAGATGAACGTAAAAACCGCTTTCAT |
| | | AATGATAATGCGGCGCTCTATGAAACACTCGCGTTGGATCGTCTGTGTGTGCTGATGGCGCGCGACTGTTTGGTT |
| | | CGTCTGAACCGGAACCTTGAAAAACATGCTACGCGCATATCTTGGGAAGCTACCGATGCGGGTGATACAATCTG |
| | | CCTCGAATTGCCGCGCCGTGATCGTGATCATGAAAGCTTTAGACTGTCTTTTGGGGTCCGTGATTATCCTAAACT |
| | | GTATGTAATGGATGATCCCGTATTTCTGTGTGGTTTAATGAAGCACTTTTTCCCGGACAACCAAGCAATTCAGTA |
| | | CCATGAGCTGTACTCGGAAGGAATCAATAAGTATACGGCTATGCAGGCCGAAGGTATCGCGGCTACTCTGAAGC |
| | | TGGAAGAGAAAACGATAAAAGAAAAAAATATGCAGATACCAGCCACCGGTTACATTCGCTTTTGCGAAATTGTT |
| | | AGCCAGTCGGACTTCGCACCTGGCGAAAAAAGAGTGCTGAAAAACGTGCGTAATGGCTTACTGCATTATCATCT |
| | | TGAGTTTGAACCGACAGAATGGGCGGAGTTCCGAGAGATAATGAAACGCGAGGGATTTGATACTGCGAAGAAA |
| | | CGTAAAAGTACCCGCAAAAAGTAA |
| 315 | JW13138 | ATGGAAACTCAGGAAAATGAATTGTTTGAGAATCGTAGACGGACACTCGCAGATGATCCTCAGTACTTTGGCGC |
| | | GTACCTTAATCTCGCTCGTCTGAACATCTTCAGCATTAACAATTATCTGGCGAAGGAATTCCAACAAACAGAATT |
| | | GCGTGATGACGAATTAATCCAGAACGGTTTCATCTGCATTCCCAGCCAGAAAAATCGTAACGACAACAAACTGT |
| | | TCGAACGCTTAATCCGTCTGATGCCAGTAATTAAACAATTTGATGCGGCCAGACTGCCGAATGAAGAACAAAAA |
| | | AGTGGTCAAGCCGAAGGTAAAGATATAAAGACTATGGCCAAAACGCTGAAAGCTCTGTTCAAGGATATGCAGG |
| | | AATTTCGGAATGACTACACCCATTTTTATGCAACAGATAAACAGGAATGTCGTAAACTTACGGTCAGTAGCGAA |
| | | ACCGCTACTTTTCTTAAAGCCAGCTTTGCCCGTGCCATAGAATATTCGAAAGTACGTTTTAAAGATGTGTTAGCA |
| | | GAAGATGACTATTCCCTTGTGGCTGGTAAAGAACTGGTCGAGGATGAGAACACCATAACCACCGAAGGCCTGGT |
| | | GTTTTTTACCGCGTTGTTCCTTGACCGTGAGAATGCGTTTCAATTCATTGGAAAAGTGCAGGGGCTGAAAGGCAC |
| | | ACAGTTCAAAAGCTTTATTGCAACTCGCGAGGTGCTGATGGCTTATTGTGTCCGTTTACCGCACGACAAACTTAT |
| | | CTCTGATAATCCTATCCAGGCATTACTGTTCGACATTTTGAATGAACTGAACAAGTGCCCCAAGAAGCTGTACAA |
| | | TGTTCTCAGCGAAGAAGTGAAAAAAGAGTTTCGTCCAGAGCTTTCCGAGTCAGGAATTGAGAATATGCTTGCCA |
| | | ATAGCTTTAATGATGAGGAACGAGAGAAGTTGTTGGACGAGATTGAATATAAAGAGTATATCGAGCAGCTTACG |
| | | ATACGTGTGCGGCATAGTAATCGTTTTCACTACTTTGCGATGCGCTTTATAGACGAGCAAGAATTATGGCCAGGA |
| | | ATTCGCTTTCAAATCGATCTTGGCAAGTATACGCTGACACAGTATGATAAACAGGTTTTAGGGGAAAGCATACC |
| | | ACGCTCGGTGGTAGAAAATACTCGGGCATTTGGTCGTCTGTCTAACTTTCTGACAGCCGATCCTGACTCTCTGGA |
| | | GCAGCGTATTGATGTTGATCATCGGGCCAATGGCTTTGAACAATTTGCGCCCCATTATAATGCAGATATTAATAA |
| | | GATCGGTATTGCATTCACAGAGAAGGAACAGTTGCCGTACGTCGTACCGACAAAAAATGCGGAGACCAATCGA |
| | | GTCAAGTATTCCTTTAAACAGCCACAGCCGGACGCCTTTATTTCGTTGCATGAGTTGGCGAAAGCCATTTTACTG |
| | | GAACAGTTGCAGCCTGGCGAAAGTGAAAAACTCATCCGCGCATTCTGCTCTACAGCAGAGAATAAGCTGTTTAA |
| | | TCTCGAATTCATCGAAAGTATCAAGTGGAAACTGCCGGATTGGAAACCATTCCAGAAGCAGTGTGATACCAAAA |
| | | AATCGCGTGCCTACATTACCAAAAAAAAGATGTTCGCTTATGCCCCACGCGATCGGAAAAAATTGCTGGATGAA |
| | | CTGCTGGCCCCGTATGGCCTGAACTGGAAACAGGTACCTGAACGTATACTGAACTATTGGTTAAACATTGAGGA |
| | | AGTCCATGATGCGCGATCAATCAAAGAAAGAATTTTACTCATGAAACGTGATGGGCTGAAACGACTTAAACAAC |
| | | TGGAAAAACATCGCAAAGATCCTTCTATTAGAATCCCGAAAGTTGGTGAAATGGCGACGTTTTTAGCAAGAGAC |
| | | ATTGTGGACATGATGATTGATAAAACAAAGAAACAGAAGATCACATCTATCTACTACGACAAAATGCAAGAAT |
| | | GTCTGGCGTTTTATGCTGACGCAGAGAAGAAACGCCAGTTTATAGGTCTGGTGCGCGAACTGAACCTGAACGCA |
| | | CCGGACGGACACCCCTTCCTTAAAAACCTCAACTTGGAAAGTATTCGCAAGACGTCAGAGTTTTACAAGTTGTA |
| | | CCTGATGGAGAAAGTTGAGAAAATGATAGAAAACGGCTATAAGTCCAACGGTGACAAACGTTTTAAGAACATC |
| | | GCTTGGATCATACGAGTTTTCGAGGAAAAGCAGAAAAACGAAAAAAATGGGAAAGATGACACCATCTATCGCA |
| | | TCCCAAATAATCGGAATGATATTCCGTACACCATCCGTCAGTGGGAGAAAGAGACTTCTACTCTCGAGGAATGG |
| | | CTGCATACGATCAATCATGGAAAACAAGAGGGTGACCGGAAAAGACCTATAGATCTTCCGACCGATCTTTTTGA |
| | | TGCCCGTTTATGCGAATTAATGAAAGGACAGATGCAGGAGAATGGCCAAACGTTCGCCGATACAGCTAAGTATA |
| | | ACGAACTGTTAAAACAGTGGTGGTCCTTGCGGGAAGATGAAACTCAGCCTTTTTACAATGGAGCCCGCCACTAC |
| | | GTTATTAAAGAAGAAGCTGTGTGTTTTATGCCTGGTGCCCAGCCGCGACTCACCAGTTGCTATGCGGATGCAGTA |
| | | GAGGAAGCTTATAATAGACTGAGTGCAGAACGGAATATAGCGCGCCGCCATAACAAACAATTACCTTTAATCCA |
| | | AAAGCAGAAAGTGGAAAAAGTATTCAAGCACACTTTATATGACACGGAGCGTGAAATACGACTGCTGGGCGAA |
| | | CAGGACAGAATTCTCTTATTAATGGCAGAGCAGTTAACACCGGGCCGTGAAACCCTGAAGCTGAAAACCGTGTC |
| | | AGAAGAACTGACACGCCCCCGCGAGATTAAAAAAGAATTCCCGTATATTTCTAATTACAACGCGCAAGGGGAG |
| | | AAGCTGCCAAAAGATGAACCGAAGCCGAAAATAAAAGTCACGGTTACGGGAATTATGCAACTCAAAAATTCCA |
| | | ATGATCTTAATAGATTCGCGCACGACCGGCGCATGCCAGAGTTAGTTGCATATCTTCCGAATGCAACCATTGATG |
| | | CTGATACCCTGAGATATGAGTTAATGGATTATAATAAAGCTCGGTTGACCGTTTTTGACGCTGTATTTCGTTTAG |
| | | AAAAAGCTATTATTGATCAGGATCGTGAAGGTGTTATTACTTTATTCACTGATGAACAAGGCGAAGCCAAACAA |
| | | GGGCGCATGCAGCATAAACCTTATCTTCAGTGGTTACTTAACAAGGGGATCATTACTCAGCCAGAATCATCGTA |
| | | TCTTAACATGCTGCGCAACTCATTTTCACACAACCAGGTCCCCCATATAGCAGTTGCTCTCAACGGTACGGAAGT |
| | | TGTCCCCGTTAAAAATATTGCATCAACGATTGCGGGTGCGTATAAACAAAAGATTGAAACTATTATTGGTAAAA |
| | | TAGGGTAA |
| 316 | JW13139 | ATGAAAAAAGAGTTGAACAGAGAGGATGTTTTTACTGGGGGCAAAACCCCTGAACTGACGATCTATTATAATAT |
| | | AGCTTATTTCCGTATGGCCGGTCTGATTAATGCGCTGTGCGGCAATAAACTTGAGCGAGATAAGGATGCGCTGG |
| | | AGCAGTTCATGAAAGCCTTTATAAACGGGAAACAGAAATTAACGGACATGCAGTTTGTGAAACTTTGCGACTAT |
| | | CTTTGGAAAGGCTACAAGTATGATAAAAACCGTAGCGAGTATTCGCTGACTGAAAACGATAAAACGATGGTCCT |
| | | GAAGATGGTAGCCAAACTTCAGGATATTCGTAACTTTCAATCACATATCTGGCATGATAATAAGGTCCTCGTGTT |
| | | TGATGCGGATCTGGTCCAATTCATTAAAAATAAGCATCTGGAAGCGATTGATGCACAGCGCCAGCGCTTTTCTA |
| | | AAGAGACAGAAGTGTATGAGAAAGAAAATCGGGAGCTTAAAAAGAAAAAGAACAAGGAGTTACTGTTTGACAT |
| | | CCATGATGGATTGGGTTATATCACAGGTGAAGGTCGCAACTTTTTCCTTTCATTTTTTCTTACACGCGGGGAAAT |
| | | GACGAGATTTTTAAAACAGCGAAAAGGATGCAAACGTGATGATACACCGGAATATAAGATTAAACATCTTGTTT |
| | | ATCGTCATTTTACTCATCGCGATGGAGCGACAAGAACTCACTATGGTTATGAGGATAATATGTTAGATCAGTTGC |
| | | CGGATAAACAGGAGTTTCTTACCACGCGCCATACTTATCGTCTGATCAATTATCTTAATGACATCCCGGAAGAGG |
| | | TTACGAATCCAGAGTTGTTTCCGCTGTTCAATACCATTAGTAACGGGATTCTCATAAAGGAAAGCGTCGGCACTT |
| | | ATATCGCCTTTATCAATAAAATGCCTTTGTTATCGGACTTTGAGTTTAGTGAAGTCTGGGGCAAAGATGGTAAGC |
| | | CGTATACTAACCTGGTAGAATTTTACCATAAAACACAACCTGGGTTTAAGTTTCGCACAGATCTTAACAGTTTCC |
| | | ACAAAATCATTTTAAACTTAATTCGGGACGAGAACTACACGGGATTCTTTACGACCCAGCTTAACCTGTTCATGG |
| | | CACATCGCGAGGAGCTGATTTTAGTTATTTCGAAACCACTGATCACACCTAATGATCTGTTGCTGATTGAAGAAC |
| | | ACTATCGGTATAAACTCAAAGCCAACGATTTCGTCCGGGAAAAACTCGGTGAATGGAAAGAAAAGATTGAAAA |
| | | AGGTAAGTGGGAAAAAGCTAATGAACAAAAAGACAAACTGATCAATCTTTTGGGTGGCGTAATCGAGATAACC |
| | | TTTTACGATTTTTATTGGCAAAAGGACGAAAAACCCCGCAACGAAAATCGCTTTATGGAATTTGCTATTCGGTTT |
| | | ATGGCTGACTTTAATCTGTTACCGGACTGTGAATGGGAAGTTGAACCATTACAGATAGAAAATGATCTGATTGC |
| | | AAATCGGACCGCAACCCCGGATTTGAAAAAATCGGGTTCTCAATTCATGAATCAACTGACTGATAACTATCGTT |
| | | TGAAATTCAATGATGGTCAAATAGCTTTTCGTTACCAGAATCAAGTGTTCATAATGGGGCACAAAGCGGTTAAA |
| | | AACTTGCTGATAGCCTGTTTTGACGGGAAAGAAAAAATGTTAAACGGCTTTATGCCAGCGCTGCTCGCAGACCT |
| | | CAAGAAAATTACCAATGAACATATTCTGAAAAACCATCAGGCGCTGAACACGCTCTCACTGCTGAACAATGATA |
| | | GTATACCCAGCTATATTTCTCGCCAGTGGGGTGAGGCAGAGCCCATCACGGATATGAAAAAAAAGGCCATCGCG |
| | | CGAATGGACTATATTACACAGCAGTTTGAAGCATTGATCGAGAATCACCATTACCTGAATCGAGCGGATAAGAA |
| | | CCGACAGATCATGCGTTGTTACAAGTTTTTCGAATGGCAGTATCCCCAGAATTCCCAATTCAAATTCTTACGGCG |
| | | GAATGAATATCATCGTATGTCTATCTATCATTATTGCTTAGACAAAGAGCAACACAAGTATGACAAAAAAGGGC |
| | | ATAACTACCTTTATAATCGGCTCATTAAAGAAAGCCACAATGAGTCAGGCAATATTGAGCAACACCTTCCATAT |
| | | CAAATTCGTACCATGCTGAACGATGCAAAAGATTTCAACGACTACTTCCTGAGAATCCTTAATGCAACCCATAA |
| | | GATTCTCACCGACTGGAAAAATCAGTTAAAACAGGGACGAGAACCAAATAATTATTATCTGAGTAGACTCGGTT |
| | | TCACAGGTGGCTTAACTCAGAAAGTTGTACACACCCGCCTGTTGCCATTTTCCATTCATCCTGGTATTCCGGTGT |
| | | CATTTTTTTACAGAACAGAAATGAACCAGAATCCTTCTTTTAATCTGAGCGCAAAAGTGTGGAATAGCGAATCC |
| | | CCATTTCGTGTTGGACTGAAAGAATCCAACTACCAATACGCTAAGTACCTCGGACTGTTCAATGAGATCAAAAC |
| | | CCAACGTAAAATCATTGGCAAGATGAATCAGTTAATAGCCGAAGATGCCTTACTGTGGCAGATAGCGAAAAAGT |
| | | ACCTTGATAAGTGTCATCCCAAGTTTTCTGAAATAATCAATCAGCAGGTAAACGGCAAACAGCCCTTAAACGTC |
| | | AAGGCCATACACGACACGGAATTAACAATTGATTTTCCGGTTGACAAAAACTTGTATCAGGTGGTTGTGAAAAT |
| | | GAAACAATTGGACGACTTCATGTTTATAGATTCAAAGCCGGTGCTGGAGAAAACCATCCGCTACATGATTAAAA |
| | | ATAGTTTACGTGACAAGGAATATCAACAGCGTGTGTTCATAGAAACAAATCCTGGCGTTTTCAAGATTCCGTAT |
| | | AATGAAGTGGTAGAAGCTATGCGCACTATTAATCATCAAGCACTGACTTGGGCTAGATTACTGTTAGATTGGGA |
| | | ATCCAAAACAGTTAAAACAATTCCGGAAGCAGACAAAAAAGCAAAACTGCAGGAAGCGTACAACAAGCGGCA |
| | | GCCGGCCTTCTTTAATTTTAACGATATATGTAGATACGCCGATGATGGAAAACAGTTGAAAGATCTGCGCAATA |
| | | AAACTTTTCACACCGAAGTACCTGAGACCTGGACGTACGATGAGATGCGTGAGAACAAAACGATCAAAATGGT |
| | | CCTGGGCATTAACGAAACCACCTTCTTGAAGAAAGATTACGCTATTGAAAATGCTCGTACTATTGAATAA |
| 317 | JW13140 | ATGCGTAAAAATTATCAGACGAAACCAAAAGCCCCAATTTCAACTGATCAGCTGTTCACCGGTGCAAAAACTCC |
| | | GGAGTACGCAGTGTTTTACAATATCGCCTATTGGTGGATGTCTGAAATGTTAAAACGATTATACAAAAACACAG |
| | | ATACCAGCATCCACGAAGATACTTCACCCTTTATCCAGCGATTTGTTAATGATGGGGAAGAAATTAATTCCAGTA |
| | | TTTTTAGTGATGCAGACTACATTAAGCTGCGTGACTATCTGTGGAAAGGCTATCTGTTTGATAAAAATAAGTCCG |
| | | GCTCTATAATAACCTTCAAAGACAAGGTAATTATTCATAAGATTATTGCGAAACTGGCAGAAATTCGGAATTTC |
| | | CACTCCCACTATTGGCATGATAACAGCATACTGAAATGTGATGAGGACTTGAAATCTTGGATTGATTTTCTTCAT |
| | | AATTACGCCGTCGATAAACTTCGCGTTGAATATTCTAAAGAAATCGAAAGATACGAATCGAATCGAGATAATTT |
| | | GTTTGACATGCATGGTTCGGATTATTTTATTACTCAAGAGGGCAGAAATTTTTTCCTTTCATTCTTTCTGAAAAAA |
| | | AGCGAGATGAGTCGCTTTATGCAGCAGCGTAAAGGTTGTAAACGCAATGATAAACCGGAGTTCATGATCAAGCA |
| | | TCAAGTATATCGTTATTATACGCACAGAGATGGAGCCAGTCGCAATCGTTATTCAATAGAAGAAACTGTTTTTTC |
| | | TGCCCTTGACCCAGATTCTAAAAGTGAAATTAACAAAGCCCGTCAAGTTTACAAAATGTTAGCATATCTTAATG |
| | | ATATACCTTCCGAAGTCATTGACAAGAAGCTGTTTCCGCTGTTGCTGAATGACGAAATTGTTGAAGATTCGACAC |
| | | AGATTATTAAGTTCATTAAGAAACACAACCTTTTCTCGAATTGCAAATTTGGTATTATCGAAGATTCTAAAGGGG |
| | | AAGTCATCCAAAAAGAGGTTCTGTTTAGTTATGTGGATAGCAAGTATAATTTCCGAATACGTATTGCTGCTCTGC |
| | | ATAAGCTTATCCTGGATATTATTCGTAACAGTAAACGCGAAGAAGAATTCTATAATACCCTGAAAACTTTCGGG |
| | | GATTTTCGTGATCAGTTTCCAATTATCTTAGACGATATCAAAAAAGGTATCGGCAATATTGAGGAACTGAACGA |
| | | ATACTATATTTTTAAACTCAAAGCGGATGACAAAACCAGAGGACTGTTAGGTAAAGTGATTGACGACTACAATA |
| | | AGAATCAGTTTATCAAACTGAAAGACTATAACGAACTGAAAGAGAAAAGCGTGGACGAGCCAATCCAATTGAT |
| | | CTATCATAATTTTTATTTTGAATCAGGTCAGAAACCTCGCTCTGGCAATCAATTCATGGAATTTGCTGTTAAGTAT |
| | | CTGATGGATAAAAATCTTGTTCCGAATTGGGAATGGCTCTTAGAAAAGTATGAGTCTGAGGAAGTTGAAAAAAA |
| | | ACTCGAGAACAACAATCCGAAGAAAGTAGTGAAACACATAAAAGCCTATTTTTCATATATCCCGGATGGTTATC |
| | | GCCTGCACTTCAAGGAAGATCACGTTCTTCTGCGTTTAAAATACGAGTCAGAAATTATCATTGAGGTGGGGGAA |
| | | AACGCGATGAAGAATCTTTTAATTACTCACTTTCATGGCAAACCTATCAATGAAGTCGTCGAAAAGACCGTAAG |
| | | CGATATAAACAAGATAAAACACGTGGCGAAAAACAACAAAACATTAACAGAGAAACTTGAGCTGTTAAACGAC |
| | | AAGACTATACCTCGTTATTTGAAACTGATGATGCATGACGAAAGTGTAATGCAGAATGAGACAATTAACTATAG |
| | | CATTATACAGGCGAAAAATCGCATCGATCGCCTGATTGAATACTTTGAAGATCTTCATTCAGATAAGGTCAAGC |
| | | TTAATCGGAGAGAGATTAATATCCAGCTCATCCGTTGTTACAAATATTTCGATTGGAATTATCCGAACAACTCTG |
| | | AGTATAAGTTTCTCCGGCAAAATGAATACCAATTAATGTCAATCTACCATTACACGTTGGCCAAACCGAAAAAC |
| | | GCTAAACACCATCTGTTGATACTGCGGAAGGAACTGTTTGGTGACATCAATAAACATCTGCCGACCAACTTGAA |
| | | ACTGATCCTGGAAGAGGCCACATCCATCGATGAACTGTTCAAAACGGTGTTACTGGAAACGATAAGCCGCCTGA |
| | | ACAAATGGAAAAATAGATTCAGTTCTATGAATTCGAACCAGAAAAAAGATTGCCTCAGTAAACTTGGTATACAT |
| | | ATCCCGAAAATCCCGGTCCCTGACGCGTCAAATAAAGCAAATCTTGGCCAGATGAAAAACTTGCCCTTTATGTT |
| | | ACACCCGAGCTTAGTTTTTATGACTTTTAAGTATTCCCGTTCTAGCACCATATTCTCAGAATACCGGGACAATCC |
| | | CAAAAACAGCAAGGGATTGTTAATGGATCATTATAACTTTCAGAGCTATCTCGATTTGCATTCCGTTTTAATAGA |
| | | TCAGAAACAGGCTCATAAAATTGTGGGGACGATTAATAACCTCAAAACTTTAGACACGTTAATATGGCAGATGA |
| | | CAAAAAAGTACCTGGACGATACCACGAGCGATATATTGAAAAACATTACCAATGAGCTGATTAACAGCTCCACA |
| | | GAGAACTTCAATGTTAATCAGTTGCATAACTTTGAATTTCCCATTTTTAAAAGTGACAAAAGTGAGGTGAAACCT |
| | | TTCAAAGTGATAGTCAAGTTCAAACAGTTAGCGGATTTCATGCTCATTACCGAAAAACCTTTAAAAAATGTAGC |
| | | TCTGCAATTATTCAAGCGTTATCAAACCAACGAGGAGTTGGAGAAGCTCGAGGTGTACTTTAAGAATGGATTTT |
| | | ATGAGATACCCTATGTACAGATTAAGCAGGAAATGGACCGCATCTATCGGGAATCCCTGATTTTAGCAGATTTT |
| | | ATTTTGGATTGGGAGAAGTCGATCGTGGATAAAATCCCAGAGATCGAAAAGCAAAAGCTTATCGAATCGGCGA |
| | | AACAAAACACAAATGGCTATAAGGCGTATATCAATTTTGATACGGTTTGCAATTATGCTGGACTCATTAAAGAA |
| | | GAACAGTCAGAACTGCAGAATCTGCGGAACCATGCACTGCATGCTAAAATTCCAGATAAAGATTGGACATATAA |
| | | ACAGAAACTGAACGACAACCGCATTCAAAAAATGCTGTACATTACCTTAGAAAAACTCGAAAAATACGAAAAA |
| | | AAGAATGCATACTACGAGTCCAAGGTACTTCATAACACATATTAA |
| 318 | JW13141 | ATGGAAAAAAGTAAGCTTGATAATGAGATGACGTTTGCCGAGGATGCCTATTACTTTGCGCATTATGCAAATAT |
| | | GGCTCGCCATAATGCGTATCTTATTCTGAATCACATCAATAAAAGTATATTTGGCAACGAGTCTACCTTAAAAGA |
| | | GGAGAACCTTCAGATTGCTATTATTAAAATACTTGATCCTAAAAATGGAGCCGACCACCATGCTAAAGCGATTA |
| | | AACTGTTGAATCATCACTTCCCGTTTATCGCCTGCTATTATAAAGAAGATATTAAAAAAGCCGTGGAGAATAGC |
| | | GCATACAAAGAGATGAATGACAAAAAACTTGATCACCTTAAGGAACCTCTGACGCTGAAACAGATACTGAAGA |
| | | CCCTGACTGGCATCCGTAATGAACTCAACCATTATGGGTATTCCCGTAAAGAACACCCAATCCCGTTCTTTCAGG |
| | | ATCTGTTTAATGACTCAATTCAATTGGCGAAAAAACGCATGATAGAGTTCAATGAAACCCGTTTTAATAGCATTC |
| | | AGGATTCACCGTATCATAAAATGGTTGATCCCAAAGATTCCGTCCTCACGGACACCGGCAAACTGTTTTTCTTGT |
| | | GTCTCTTTCTTGACAAAAAGAATGCGTTCGAAATGTTGGCGAAAGTAAAGGGTTTTAAAAATCGGACCGACGAA |
| | | AAATACCGTGCAACCCTGGAATGTTATACGCAATTATGTTGCAAAACTCCCTATCCGAAACTGGAAAGCAGCGA |
| | | TATCAGTTTGGATATATTAAACGAGTTGAACCGTTGTCCCAATGCCTTATATCAGGTGCTGTCTGAAGAAGATAA |
| | | GCTGAAATTCAAAAAAGAACCAAATGAAGAAACTGCGGAAGAGTTAAACCCTATTATGAAACGTTATGATGAT |
| | | CGTAGTGCTTATCTGGCACTTCGGTATTTTGACCGTACCAAGTGTTTAGACAATATAACATTTCATCTTCAACTCG |
| | | GGCGTAAAGTGATGGGTGAACCGCACAAAAAAGTGTTTGACGGAAAGGAACGCACCCACCAAATTTTAAAAGA |
| | | AATGCGAGTACATGATAAACTTAGCACGTATGAGACTGAAGAGGCCCAGACATTTTTTCAGTCAAAGATTGAGT |
| | | CCAACGAAGTTGAATTATTTGCGCCCCATTATCGGATGGTTGGTAATCGAATTGGCTTAAGTTTTGGTACTCCGT |
| | | CCGAAAAAAAAGAAGGTTATTCGATCCCTATTAATAATCAGCCAGCCCCGCAAGCTATTTTGTCTACCAGTGAA |
| | | TTGGGTGCATTATACATCTACAATCGACTGTACAAGGAAAATAAAATCACTCAATCACCGAAAAACCTGATGTC |
| | | CACTTATCTTCAGACCATGAACCGTTTTATTAAAGATTGGAAAAACGATGGTATCAAACCGGAGACCAGTTCGA |
| | | ACAAAATCATTAAGGAACGTAATCAAGAACGTACGAAACAGACGATCAATGAATTAAAACGACGTACGGATCA |
| | | TCTGCAGAAGCTGCTGGACGAGAAATATGCAGGGCTCAAAGTTAAGCACCTTCCGGATATTTGTAGAGAACATC |
| | | TGTTAAACTATCAATCAGACGATAACATACAGCAGGCAATTGACTTTCTTAAGAAAACCCTGTCAGACACCAAT |
| | | TTCAAAAAAAAGCAGCTGGAACAATTTGAGCGGAATATGGTCTACCGGAGCAGCAATTCAAAACAGTTAAAAA |
| | | TAGGCGAAATTGCACAGGAATTGGCACGCGATATAGTCTTCTTTTTGAAACCTATTAAAAAGGAGATAACCAAC |
| | | CGGAATAAAGAAAAAGTGACCGTGTTCCAAAAAATCAATAATCAGGACTTCGATATCCTGCAGAATCGGCTGGC |
| | | TTACTTTTCCCTGTATAAAGAAGAATTGTGCTCGTTCTTTGACGATCTGCTGAAGAAGAAGCACATGAAACATCC |
| | | ATTCCTGGAGAACGTCAAGGCAGAAACTAAGTGGAGTAACGGCCTGATTGAATTCTATCTGAAATACCTTGAAC |
| | | ATAAGCTCAAATGGCTCAACACATACACAAAAAATGCTCCCAATAATACAACACGCCCAAATTATACAACGAGC |
| | | AATCGTATTAACAAGAATCGCTCTACTCCAAATAAAAAGGATGAGATAGAGGAGAAGGAAATCGACCCTGAGA |
| | | AAATCAAGGAGACATTCATAGAGAAGTACAACTACTTTCTGAAAGTGAAAACCTCGTCCCCCAAAAATTATCAG |
| | | GGGAAGGCTATTTTTATTCCTGTCGGGTTATTCAATGACGCGATTACAAAAGCGATGAAAGAAAGTGGCTATCA |
| | | GGTTGAAGATGATGAAAAAACACCAAATGCTGCTTATTGGATAAAAAGTTATTTTCATGGTGCAACACAGCCTT |
| | | TTTATCAGTTACCGAGATATTATAATCGTGCCAAAAAAGATGAAGAGCGCGATCTGGTTGAGGCGGCGCTGCTG |
| | | AAAAAAGAGATAAAAAACTACTACGTTAAAGCTAACAAAAAGACGCAGAATGACTTTAAGCAGTTAGCCAAAC |
| | | GGATTAGAAAACAAGAGGAAGAAATCCTGACACAACAAACGTCGGACCGAGTTTTATTCATGATGGCAACAGA |
| | | TTTGTTGAACGGTGGTTCTGATGGACGCCAGACCATAGAATGCGAACAAATTAACACGTTAGGGTTCAACCTTG |
| | | GCGAAAACAAAATATTAGATGAGAACAGAAAAGTATCTATGAACATCTACGGACGCAAAGTTGTATCAGAACT |
| | | TCCAATTAAACGTTATGGCGAATTTCGCCGCTTTATGAAGGATCGCCGCCTGGAAAATCTGATCAATTATTTCCC |
| | | GGAAGGAAAAGATATTGAGTTAGGCGTATTAAAATCTGGTGAAGTTGAAGCCGTTGGAGAAAGATTTTCTAGCT |
| | | CTACTCTGGCCAACGAACTGGAATTTTTTGATTCTCAGCGCTGGTTACTGATGAAATCAATCTACGAGTTTGAAA |
| | | AAAAGATCTACACTCACTTCTTCGATGTACTCACCAAGAATGTGCCGGAAAAGGAGATGATGGAGAACAACTAT |
| | | ATTGCCCATTATCATTACATTGAATGCGCAAAAAAGTTAGAGTTATCCGTCTCGGAACTCGAGAAGGACGATTTT |
| | | AAAGAATTGAGAAACAAAATCCAGCATAACGAAATTCCTTACTCACAGTGGATCAAGAATAGAATTAGCACTA |
| | | AAGATGGTCATCTTATCACAAAACAGATTGTGGAGCTGGTGCTGAGCATCTATCAGCAAATGAACAACGAACTC |
| | | GATCAACTCATTCGCAAGTAA |
| 319 | JW13142 | ATGAAAACAAATCCGTTAATTGCATCTTCAGGGGAAAAACCGAATTACAAGAAATTCAATACCGAATCTGACAA |
| | | ATCCTTCAAGAAAATATTTCAAAACAAGGGTTCAATTGCGCCAATAGCAGAAAAAGCGTGCAAAAATTTTGAAA |
| | | TAAAAAGTAAAAGTCCTGTGAACCGTGATGGTCGTCTGCATTATTTTTCGGTGGGACATGCGTTTAAAAACATTG |
| | | ATAGCAAGAACGTGTTCCGTTACGAACTGGATGAGTCCCAAATGGACATGAAACCCACACAATTTCTGGCACTC |
| | | CAGAAAGAGTTTTTTGACTTCCAGGGAGCATTGAACGGCCTGTTAAAACATATCCGAAACGTTAATAGTCACTA |
| | | TGTTCATACGTTTGAGAAACTGGAGATCCAGTCCATCAATCAGAAGTTGATCACGTTCTTAATAGAGGCGTTCGA |
| | | ACTGGCCGTAATCCACTCATATCTGAACGAAGAAGAATTATCCTACGAGGCTTATAAAGATGACCCACAGTCCG |
| | | GGCAAAAACTTGTCCAGTTTTTGTGTGATAAGTTCTATCCTAATAAAGAACACGAGGTGGAAGAACGTAAAACC |
| | | ATTTTGGCGAAAAATAAAAGACAGGCCTTAGAACATCTGCTGTTTATTGAGGTAACCAGCGACATTGATTGGAA |
| | | ATTATTCGAAAAGCATAAGGTGTTTACCATCAGCAACGGAAAGTATCTCTCATTCCACGCGTGCCTCTTTCTGCT |
| | | GAGTCTGTTTCTGTACAAATCCGAGGCTAACCAGCTTATCTCTAAGATTAAAGGTTTTAAACGTAACGATGACAA |
| | | CCAGTATCGGAGCAAAAGACAGATCTTCACGTTTTTTTCTAAAAAATTCACCAGTCAAGATGTTAATAGCGAGG |
| | | AACAGCACCTTGTAAAGTTTCGCGATGTAATTCAGTACCTGAACCATTATCCGAGCGCCTGGAATAAACACCTT |
| | | GAATTAAAATCAGGTTATCCGCAAATGACTGACAAACTTATGCGCTATATAGTTGAAGCAGAAATCTATAGATC |
| | | TTTTCCAGATCAAACTGATAACCACCGCTTTCTTTTGTTTGCCATCAGAGAATTTTTCGGCCAGAGCTGTCTGGAT |
| | | ACATGGACGGGAAATACACCGATCAATTTTTCCAACCAGGAACAGAAAGGGTTCTCGTATGAGATCAATACTTC |
| | | GGCTGAGATAAAAGACATCGAAACTAAACTTAAGGCTTTAGTCTTGAAAGGGCCGTTAAACTTTAAAGAAAAG |
| | | AAAGAGCAAAATAGATTAGAAAAGGATCTTCGTCGAGAGAAAAAAGAACAGCCCACCAACCGCGTAAAAGAA |
| | | AAATTACTCACGCGCATTCAGCACAATATGCTGTACGTTTCGTATGGTCGTAATCAAGATCGTTTTATGGACTTT |
| | | GCTGCACGGTTTTTAGCAGAGACCGATTATTTCGGCAAAGATGCCAAGTTTAAAATGTATCAATTTTACACTAGT |
| | | GACGAGCAGCGCGACCACTTGAAGGAACAGAAAAAAGAATTACCCAAAAAAGAGTTCGAGAAACTCAAATACC |
| | | ACCAGTCCAAGCTTGTTGACTATTTTACCTATGCAGAACAGCAAGCACGATATCCGGATTGGGACACCCCGTTTG |
| | | TTGTTGAAAACAACGCAATTCAGATTAAAGTGACGTTGTTCAATGGCGCAAAAAAAATCGTCTCAGTGCAACGG |
| | | AATCTGATGTTATACCTGTTAGAGGATGCCCTGTATAGTGAAAAACGAGAAAACGCAGGCAAGGGTTTAATTAG |
| | | CGGCTATTTTGTTCATCACCAAAAAGAGTTAAAAGATCAACTGGATATTCTCGAGAAAGAAACTGAGATATCTC |
| | | GCGAGCAGAAACGGGAATTCAAAAAACTGCTGCCAAAACGTTTACTCCATCGCTATTCACCGGCCCAGATAAAC |
| | | GATACCACGGAATGGAACCCAATGGAAGTAATACTGGAAGAAGCGAAAGCGCAGGAACAGCGGTATCAGCTTC |
| | | TTCTGGAAAAGGCGATTTTGCATCAGACCGAGGAAGATTTCCTTAAACGCAATAAAGGTAAACAGTTCAAACTG |
| | | AGATTTGTACGCAAAGCCTGGCATCTGATGTATCTGAAAGAATTATACATGAATAAAGTCGCTGAACATGGTCA |
| | | TCATAAAAGTTTCCACATTACAAAAGAAGAGTTCAATGACTTTTGCCGTTGGATGTTTGCGTTTGATGAAGTCCC |
| | | GAAGTACAAGGAATACCTGTGTGATTACTTTAGCCAGAAGGGATTTTTCAATAATGCCGAGTTCAAAGATCTGA |
| | | TCGAGTCAAGCACGAGTCTTAATGACCTGTATGAAAAAACAAAACAGCGCTTTGAAGGATGGTCAAAAGACCT |
| | | GACCAAACAGTCAGATGAAAATAAGTATCTGTTGGCCAATTACGAGTCGATGCTGAAAGATGACATGTTGTACG |
| | | TGAATATCTCTCATTTTATTAGCTACCTTGAATCAAAGGGGAAGATCAACCGTAATGCTCATGGCCACATTGCAT |
| | | ATAAGGCTCTCAACAATGTTCCTCATCTTATTGAAGAATATTACTATAAAGATCGATTGGCACCCGAGGAATAC |
| | | AAGTCACATGGTAAACTGTACAATAAACTTAAAACAGTTAAGCTCGAAGACGCGCTGTTATATGAGATGGCTAT |
| | | GCATTATCTGTCTCTGGAGCCTGCCTTAGTGCCGAAGGTTAAAACTAAAGTTAAGGACATCTTGAGCTCTAATAT |
| | | TGCATTTGACATTAAGGATGCCGCGGGCCATCACCTGTATCATTTGCTGATTCCCTTTCATAAGATTGATAGCTT |
| | | CGTGGCGTTAATCAACCATCAAAGCCAGCAGGAAAAAGATCCAGACAAAACCAGCTTTCTGGCGAAAATACAG |
| | | CCGTATCTTGAAAAGGTCAAAAATTCGAAGGATTTGAAAGCCGTATATCACTATTATAAAGACACACCTCATAC |
| | | ATTGCGTTATGAGGATCTTAACATGATACATTCCCACATTGTGAGTCAATCTGTCCAGTTCACGAAAGTGGCGCT |
| | | GAAATTGGAAGAGTACTTTATCGCCAAGAAGTCCATTACATTACAAATTGCCCGGCAAATATCGTACAGTGAGA |
| | | TAGCTGATCTGTCGAATTATTTTACGGATGAAGTCCGTAATACCGCTTTTCATTTTGATGTTCCAGAAACTGCAT |
| | | ATTCTATGATTTTACAGGGTATTGAGTCTGAGTTTCTGGATCGTGAAATTAAACCTCAAAAGCCTAAAAGTCTGT |
| | | CTGAACTCTCAACTCAACAGGTGAGTGTCTGTACTGCTTTCCTCGAAACACTCCATAATAATCTGTTCGATCGTA |
| | | AAGATGATAAAAAGGAACGGCTGTCCAAAGCTCGCGAACGTTATTTTGAACAAATTAATTAA |
| 320 | JW13143 | ATGCCTACTACGAACAACCAAAAAAAGAAAGCTGATTCAATCAAACGAACGTCTCCATCCCCCTCACGTATCAA |
| | | TGGGGTCGCAAAACCCCGCCCGCTTGTCGTTGATGAGAACAATAAACATGTTCTCGGGGGTTATTTCGGGCTGG |
| | | GCCTCAACAACTTTTACAAAGCGACCCTGAGCATATTTGCGCGTACAGGGATTAAAATTGCCTCAAAAAATGGC |
| | | ACGGTGCTTTATCCGGAAGAGAAAATAGGACAAGTGATACGGGTCCTGCGGCGTTCAATCTCGGACACTAGCGG |
| | | TAATCTTACACATGAAGAGAAAGTATGGTCGAATGCTTTTAAACTTAGTTCTTCCCAGGAGATGCGCCTGCAAA |
| | | AATTGATTTTCCATCATTTTCCGATCTTAGGTCCCATTATGGCAGACGAAGGTGCCTACAAAGCAAGCACTAACA |
| | | AAAAATCAAAAGTGACGGATGCTTACTCTATGACCTACGGCGTTACCCTGGAAGAATGTTTGGACGTTCTTGGC |
| | | GATCTTGCGCAGACCTTGGTCGATTGTCGGAATAGCTATACTCATTTTTATCCATATAACAGTAAAGAATCGACA |
| | | GCAGACCAGCTTGCGCTGCAGAGTAAAACAGCCCGTTATCTTGATAAAGTGTTTACCGCATCCCGTCGGCTCGA |
| | | TAAGGATCGTAACAATATAGATACTGCCATGATGGAGTTCATAACAGGGTCTGCTCGAAATCTGCGACAGTATG |
| | | TCGAACGTTATGGTTTTTACCCAAAGTATGACGAGACAGTGACCTATGAGAAGGATACGCGGGGACGTGAACGC |
| | | ATGAAAAAGAAGATGACTGAGAGAGATGATTTCTTTTACAAGATAGGTAACGAAAAAGAATGTGACGGGAAAC |
| | | GGTATACAGTCCTGTCTGGTTTCGGGTTATCCTTTTTTTGCGCGGTATTTCTGTCTAAAACACAGACACGTAATAT |
| | | GCTGGCCGACATACGATTATTTGAAACTTCACCGTATCCTAAAGAACTGGATGACATTATTCGCGATATGATGTC |
| | | GATCTACCGCATTCGGGCCCCTCGAGGCAAGAAAAAGCTGGAAGCTGGAAATACTGGTGTAACCCTGGCGTTGG |
| | | ACATTCTTAACGAACTGCGTCGATGTCCTAGAGAACTGTATGATGTTCTGACCACTGAAGGTCGTAAGTTTTTTG |
| | | AAGATGAAACGGACCGTACTAAAGATGCTTTCGTCCCAGATGATGCCTTATCACCGTACACGAGTGCTGACAAC |
| | | GCTAGTGAAACCGTGAAACGCATTCGATGGACTGATCGTTTCCCCCAATTGGTTATGAAGTATGTTGACGAGAC |
| | | CGAGTTCTTTAAGAACATCCGTTTTCAAGTGCGACTGGGTAAGTATCGGTTTAAGTTTTATGATAAACTTTGTAT |
| | | CAATGGAGAAGAGGATCTTCGTGGTTTACAGAAAGAAATCAATGGTTTTGGACGTATTCAGGAGATGGAATCTG |
| | | CTAGAAAGGAAAAATATGGGGACATGATGCAGCCGGTGGAATCAGAGAGAATTCGTATTGATGACGATCTCGA |
| | | ACTCGATCTTTTACAGTTTGCGGAAGATAGCCGTGATTCGAGTCCGTACATCACAGACTCTCGGACGGCCTACAA |
| | | TATTAACAATAATCGAATCGGATTGTATTGGAATGAACTGACAGTGAACGGTCGCACAATCCCGAAACCAGGCG |
| | | ACCATCTTCCTGACCTGGGATTAACTCCTGAGGGCAAGGCGGATGTCGATATGCCTGCACCGATGGCAATGTTG |
| | | TCGTTACACGAATTACCCGCGATGGTCTTTTATCAGTATCTGCTGTCCCAAAATGCTGACCCGAATGAACTGAAA |
| | | ACCCGCAGCGCCGAGAATCTTATTATTGGCAAGTTCAATAGCCTGCGCCGTTTATTCACAGACATTAGCGAAGG |
| | | TCGCTTAGCACCTCTGCCGGAAGATGGCTTAGTTCGTGTATTAGACGACCGCTATGGATTAACCCTTCGTGAGAT |
| | | CCCCGATAAACTTACGGATTTCCTGACTGGAAAAAAAGGTGCCGATCAGTCAAAACGCCGCCGTGTTGTGGCAG |
| | | AGCGCACCTTACTCGAAAGCCTGCGTAAAGCTATTCGGCGGCGCGACAGATACGAAGAGGACCGTAAGAAGAT |
| | | TGGAGAGAAAGATAACAAGTATGCAAAAAAAGCCTACTCTGATGTAAGACACGGTTCCTTGGGCCGTTACCTGA |
| | | GCGAAAGTTTAATTGAGTGGCAGCCGACATTAGAAGGGGGCAAAGATAAACTGACAGGAATGAACTTTAACAA |
| | | ACTTCAGGCCGAGCTCGCGACCTTTAATTCACCTGAGAAATATACACGTCTCAGAGAGACCCTGTCGGCGGCAG |
| | | GCATGTTATCTGGTCCTATAGCGCACCCATTTTTGCGCAACGTCACCAATCGTAAGATACGGAATATAGAAGAA |
| | | CTGTATCTTATCTATCTTGACCAAGAGATTGGCTACCTGAAAACCCTGCTCAACCTGGGTTACAAAGGCGGTCGC |
| | | CGCGACGATCGGAATATCGATTTTGAAGCGAGCACCTTGAAAACGCATGTAGATGTTCGGAAGCTGCCACTCGT |
| | | TAAGGGTGAGAAACGTTGGGAAAAAAGAACTCCCGACTATTGTCGCGAACTGGCAAAACGATATCTGGAAGTA |
| | | GATGGTAGAAGAACTTCCATTTGGTTGCCAGAAGGCATTTTCAGTAAAGCAATAACCGATCTGCTTACTCGTCAT |
| | | TATTCAGGTAATGATGGCCTGATGGATAGAATGCGCGATGATTCTTTAAACAACAACGTTGCGTATCTGATTAAT |
| | | GCCTTCTTCGAAACTGTTCTGGATGATCATTCCCAGCCGTTTTACATGAGTTATGATATTGATAAAAATTCTGGA |
| | | AAAGAAACCCCGAATAGATTTGCCAGAGTTTATGACCTTTTCGACATCCTGAACAATCGGAAGATTAGAAATGC |
| | | CTTAGTAAAAGTGCCCATGACCTGCAACCAAATTGCGGAGCGCTTTACGGCTAAAAATCGCGACGGGCGTAAAC |
| | | GCATTCTGGCGGAGATTGACGATCGTACTGCCGGGATGACCCCACGTGATATCGGCTCTCATGCTAGTTTGAAG |
| | | GAAGCGAAAGAAGCTATGCGTAGACGCTTAACGCACATGATCGGCGATGTGAAAGATAATGAACGCTGCGTGC |
| | | GTCGCTATAAGACGCAGGACATGATTCTCTTCCTCATGGCCAGTCAGCTGCTGGGGGATGCAATCCGCATGGAA |
| | | AAAATCGATAGCTTAGATAAGTTTCGACTCAGCGCCGTGTGCAATGCCGGTTTTTTGAACCAGACCATGGATTTC |
| | | GAATTTCCAGTTCAAATGGATGGAAACGTTATCTACATTCGGCAGGACAATATGTCTTTGAAAAATTATGGGGA |
| | | GTTTTACCAACTGCTCTCGGATGACCGCTTACCATCGTTATTGGAAAAACTGGCAGATGGTGGCAGCGATAGAA |
| | | TTGATTATGGCAATCTGATGGGCGAATTGACCAGTTATGACCTTCATCGGTCCCGTATCTTCAAAGCAGTGCACA |
| | | TGCTCGAACGTCGTGTAACGTCAGATCGACAATTCCGTATATTTCTGAATAATCCTTGCGACCGGAGATTTTACA |
| | | TTAACGATGATGCTAGCCGCATGGCAAAACGCAATAACTTTCGTTCCTTACTGATGCTGTTAGAACAGTCAGATA |
| | | CAGCAAGTTTGACGGAGTCCGAGCGTAATTTGATAATCTCTATACGTAATGCTTTCAGTCATAATCACTATAGCA |
| | | TCGACCTGCCGCGTATTGCGCCGGAGAAACGGCTGCGCAAAACGACGCTGCTTAACAAAGATACACGGCCGTTA |
| | | GGAGAAAAAAAAGCATCATTAACAACCATTGCTACATTGATAGCGGCACGCTTAGAAGAGTTACAGAAAAAAC |
| | | TGGAATCAAAGCACTAA |
| 321 | JW13144 | ATGAAAACAAATCAGCCTAAAACCATCTATTATAACTGGAAAGACAAAGCTGATTTTGCTTATTTTGCATTTTAC |
| | | ACGTCACAGGCTCTTAACAACACTAGTATTATTCTGAAGAACATTTCGGAGATTATAGAGAACAAGGTTGATAA |
| | | GACTAACGATGACAATCAGCTTTTCTCCAATGCACAAGTAATTAAAATTTTGGAAAGCAATAATTCTGTCGCAC |
| | | AAAAATCAGTCATTAATCTCCTGGACTCTAATCTTCCGTTTAGCGTTAAAAGTATTAGCGATCCTTTTCTGGTCA |
| | | AAAATCGCCTGTCGTACTTCTTATGGTTACTGAAAAACTTTAGAAATGAATATGCACATTATCACGAACGGGAA |
| | | CTGGATTCTCGTACCACACTGGACCGAGAAGAAGATTTCAAAAAACGCGAAAATCTTAACAAACGGTTTAGTAA |
| | | GTATGACTTCGCGGATGAATTACTGAAACTTAAAAAAAGCGCGGTTGAAGAGCTGGAAAAACGTCTGAAAATA |
| | | AACAACAAAGAATTAGAAAGCGACTCTGTTTACAAGTCTTTTAAAAATAGATTTCTGAAGATTAGCAGCAGACA |
| | | GAATTTCACGGAAGAGGACTTTATTTTTTTTCTGTGCCTGTTCCTCTCGACAAAAGAGACAATGCAGCTGTTAAA |
| | | CGGTATTAAAGGTAAAAAAAATACAACAACTGAAGAATTTCAGTGGATTAGACGTGTTTTTACCATTTTTAATG |
| | | CCAAACGTTTTCAGAACAAAATTAAAAGCGATAACCCAAAAGAAGCCTTTATTCTGAACATTGTGAACGATCTT |
| | | GCGAAAATACCAATTCATCTCAAAAAATATCTTACTGAAGAAGCAAAAGAGAAATTGATCTATACTGTGCAAGA |
| | | AACCGAGGACGAAGAAGGCAATATTTTAGAACAGAAATCTGAAGCAGTGACCAAGCATGACAAAATGTTTAGC |
| | | TATCGGTGTCTGCAGTATCTGGAACTGTTTGGGTTTGCAAAAAATATCAATTTCAACATAAACCTTGGCAAAGTG |
| | | TTCATAAACAAACCCTACAAGAAGACAATTATCAACGACGAATATGATCGTTTTTTAGATAAAGAAATACATAC |
| | | GTTCGGTAAACTGAAGGATTTTGATGACTCATATTTTGAAGGTTATATCGAAAGAAAGGAAACTGAGAATGGAG |
| | | TAGTTACCACGTTCAAACAACCACTGAAATTTTACTCCCCGAAATATCATTTTTCAAACAATCGTATCGGTATTA |
| | | AGCTGTACAACAAAAATTTGAAACTTGAACTCAAAGAGTATGATGAGGATGGCAAATTTGTAGTTATTAACAAT |
| | | CAACCCGATTATTTTCTGAGTGAGAATGCCATACCCTACTTCACCTACTGTATGATCAATTTTGGAGAGGATAAA |
| | | ACCTTAGGAGTCATTAAGAATTTCGAAACAAATTTCAAACGTTTCCTCAACGACGTTTCTATTGGAAAGAGTATT |
| | | AAAGTGCAGGAGATTGAACAGAATTACTCTTTGAAGATAGGCTGGATCCCGTCATTAATCCGCGATAATCTTTTT |
| | | CACGATAATGATAAGACGTTCGAGGATGTCGTTAAGGAAAAAATCAACTCGTTAAAAGAGGAGACGCAGAAGT |
| | | TGATTGAAAATAACAATAAGAAACCTGAAGATCGCGATCGTAATATAAAGTTTTCGTTTAAAAAGGGGGATCTT |
| | | GCAACCTTCGTTGCGAAAGACATTATCTATTTTATGGAACTGAAAGAGGAAATCGTCAATGGCAAAAAGGTAGT |
| | | GTCCAAGCTTAGTTCCATTGAATATGACGTACTGCAATCGAAACTGGCGTTCTATGGTAAACACGAAGAGGACT |
| | | TGAAACTTTTATTCAAAAAATGGAATCTTAATGAACGCCATCCGTTCCTGAAAGACGTCAGCATGGAAAAAGTG |
| | | GAAGATCGCCGGGGCTTTAAACGGCAGATTGGTATCAAAAAATTCTTTAAGAATTACATCTATCAGCGGAAATA |
| | | CTGGTTAAACGACTTGAAACTGGAGATTAATAAAGAGAACTATCATTTTGTTGACGATTACAAAGTGTGTAAGA |
| | | ATGACACTCAGATTAAAGCTTATGCTTCAAATCTCTTAAACCACACTATCTATCTTCCTAACGATCTGTTTTCTGA |
| | | TCTGATTTTGGAGAATAGTAATTTTGCCGTGGAGAAAGCTAATACGAACTTCTTGATAAGTAAAAACCTGGAAT |
| | | ATTTTGGAAATCAGTGGTTCTACAATAAGGAAAATTATCAACAGGGTCTCGATACTTATCAATCCCATCTTCGCG |
| | | ATAAACAGATTCGAAAAGCGATCACCTTAGATAGATTGTATTGGAACATGTTAAAGATTAATACACAAATACCG |
| | | GAATTCTCACAAATCTTTGAACAGAATAATCTGGCCGCGTATAATTCAGAGCAAACACTGTTAGAAAAGCAAAT |
| | | ACGCATGTCATCAAATTTTAAACTGGCCAAAGAAGATTTCCGTTACCTTGATTTTGATCGAGATTTTGAAATAAA |
| | | GATAGAGGGCTATCGTAAAATTAAAGACTTTGGTATATTCCGCCATCTGATTAAAGATAGACGCGTACCGTCCA |
| | | TTTTAGCCTTTTACACGAAGTATAAGAAACTCGGGTCCATCAATGAGGAGATCATCCGCAACGAAATTCTGGAT |
| | | TTCGAGTATTATAAGATCAGTATCTTGAAACGAGTGCTGGAGATCGATAAGCAGATCTACAACAATCTGATCGG |
| | | GAAGAATATAAGCATTCACGAGAAATTTTCCGAAAACGTAAACAAACTGTACTTGGAAAATCAGAAACTCGCC |
| | | AATAAGATAATAGAAATTCGTAACAAACTTTTACACAACAAAGTTCCAGAGATCAATTTGGAAAACATCCAGAA |
| | | GACCTTTAGTGAAACCTTATTTTTAGAAATGGAGAAATCTTGCAATGAGTTAGAACGTCTCATTTGA |
| 322 | JW13 | ATGAACTTTGAGGAGAATGGGAACAAGTATCTTTTTGGCGCCTTTTTGAACACCGCCTATGACAATTATAATGAA |
| | 145 | GTTATCTCACATATCTACGATGTCATTCGGCGCATAACCAGTAATAATCCTACTGAACAAATTTCTAGCTCTCAG |
| | | GCTTTGGTAAGTGCCGAAGAGCAGACCAGTAGTGATGAAAAAGACAAGAACAAAGAACGAATGTCCTTCGTTT |
| | | GTACGTTTCTGATTACCGGCAATGCAACATCACGCAAAATTGAATCGATGTTAAAACGTGCATTTCCTTTTTGGG |
| | | ATGATCTTATTGAAATCCAGACATCTATGAGCATGCAACTGGATAAGACCGAGTCAATTTTTCAGGTGCAGAAG |
| | | AATTGCGTGGAAGAACTGTTCACAGCTCTGGAATCCTGGCGTAATTATACCACCCATTATATTCACCCAGAAGC |
| | | AATTGCGTCCCCGACCATTAACCTGCTGCTGAAGTCGTGCTTTAATCAGGGTATCAAGGAGATCAAAAAACGGT |
| | | TCAACGCGCAGGACGAGCAGTTAAAGCATCTGTATGAGGCAGAATTTTCTTACAATCGCGAAACCAATAAAGAT |
| | | GAAGTGAAGTGCTATCACCCGGCGAACTATTATAAGTTCACGGATGAGTCTGACCTTCTGACCGATAAGGGACT |
| | | GGCGTTCTTTACGTGTTTGTTTCTTTATAAACGCGAGAGCTACCTGTTGCTGAACCAGCTGAAAGGCTTCAAACG |
| | | TGCCGACGAAGAACGTTATAGAATGACGCTGGAAGCGTTTACGGTTATGCGTTTTCATGCTCCGAAGCAGCTGA |
| | | TGAAACTGCAGCAACAGCAGATTGATAAAGTATCGTTGGGTATATCAATGATAAACGAGCTTGTGAAGTGTCCA |
| | | AAAGAATTGTATGACTGTATAGAAGACGAACAGAAAAAACAATTCGAGGTCTATCCAAATGCAGAAACTGTTC |
| | | CAGATGAACAAGAAGCCATAGGCGAATTAATTCGTTACAAAGATAGATATGAGATTCTGATGTTAAAAGCCCTG |
| | | GAACATAGCAGCGAGTGTAAAAATATGGGATTCTATTTGTCACTGGGTAAGTGCTATATGAAATGCTATGAAAA |
| | | ACATTATATCGATGGGACGACAGACAATCGTTATATTGTACATCCTCTGTACGGTTTTGGAAGATTACAAAATGC |
| | | ATCTACTGATCATAAAAACCACCTGTCGTCGTTTGGTGAAGAGTTACAGACTCATTCTCAATTACCGGTGTGTCA |
| | | TTTTGCGACAGAGGATGATGCAGCGGATAGCCCAACATCCGTTCCGTATATCAGTGAATCTTACCCCTCTTATGT |
| | | CCTGAACAATAATAACATAGGGATTAAGTGGTTGAACAGTAGTGAAAAAACGGAGTATCCCAACTTTAATCTTA |
| | | ATGAAGGTAAAGTTTCTTGTCCAACGCCGGATTATTGGTTGAGCAAGTATGAGCTGCCGGCAATGGCCTTTTATG |
| | | CCTACCTTAAAGAGAAACATAAAAACAAGCTTCAGGAATATCCTTCGATTAAAGAGCTGATCGATCAGAGTCAG |
| | | CCCGAGAATACTAAAACCTCTTCAAAAACTATCAAAAGTCGTCTCGAGGAACGACTGAAAACATATAAAGTTGA |
| | | TGCAGCTAAAAAATTAAAACGTTTAGCAGAGAAACCCGACGGCGTTCAGTCCGGGCGTCTGGCGGATGAACTCG |
| | | CACGGGATATATTATGGCTGCAACCATCCTCACCTAAAGAAGGGGGCAAAGATAAACTTACAGGGGCGAATTTT |
| | | CAGGCGCTGCAATATGCTTTAGCTCGCTATGGGTTTATGAGAACTACCTTATCGCGCATTTTTACCACTGCGGGT |
| | | TTAATTGGTAGTGGCAATGCACATCCTTTTTTAGATAAAATCAATCCGCAGGATTATTCTAAGCTTACTGCGTAC |
| | | TACGTTGGATATCTTAAACAGAAAATCGTCTATATCGACAAGCAACTGGAAAAACTCGCTAGAGGAAAAGTGA |
| | | ACATTCAATTCCACCCCACACGAAAACTTATCAAGCAAAGCCAGATTAATACAATTGAACCGGAAAAAGAGACT |
| | | GTATTTCTGCCGCGCAATATCTTCACAGAAAAAATAGAACAAGCCCTGTGTCAGATCCAGGGTAACTTTAAAAG |
| | | TAATATAGAACGCGCTAAAACCAGCGGCAAAGGTGTCAATGCATCCAAACTGATTCACTTCTACCTTGAATTGT |
| | | ATCTTAAAGACGCCGTAACGGAGTTTTACAACATAAAACGTAAGTACTCCTGTCTTTGCTACCCGTTGATTCCTA |
| | | GCGAAAAATACAATCTTAAAGAATATCAGAACACCGAAGAGAGATCAGAGCGTATCGACCGGATTAAAGATTT |
| | | TTACAACTTTAAGGAAGTGACAAAGAAGGCATGCAAAGGCCAAGAATTGAGCGAAGATGAGAAGAAACTGTAC |
| | | AAAAAGTACATCGAACTCCAGAAGGTGATCGATAATGAGAAGATGATTCGCTTACGCAAAGTACAGGATATAA |
| | | CTCTCTACCTGTGGATGCGCTTATTCATTGAAGAAAACCCAGAGCGTATTGTGTTCAATAAAAACGACCGACCA |
| | | GAGAATAGCTTATTAAAACTGAAAGAAATGGACAATACAATTCTCGACCACACGACGGCCCGCAAAATCAATA |
| | | TTGATACCCCTAATGGAAACGTCACAATCGAAGATGAATTCAAGATTAAAGATTACGGTCGTATTACCACACTC |
| | | ACTACCGAAAAATTACCGCAGTCGTTAATTAAGTTCATAGGCATTTTGAACAAACCGTTAGACAAAAAAGATCG |
| | | GCCGTCATTAAGCTATTCATATATCTACCATGAAACGAATGCTTTCTATCGATATCGCCCCGAGATTATTAAAAT |
| | | GGCTCAGGACATTGAAAACAAAATTATTAACGAACTCAAAATAGACATACAACCTGAAGCCGAGGGTTATTATA |
| | | ATTTTCGGCAAATACTGGAAGAAATCAAAGAGAAATCCTCAGATATTAATTTGGAGTTACTGATAAAGATCCGG |
| | | AATGCCTTTTGCCACAACGAATATAAAGAGCTCGATAATGATCAACATAAGTATCTCTCAAGTCTGTTCAAACC |
| | | GATAGCCGACATCAAGCAGACGGCTACTCTGGCGTACCTGCTGCACATGCTCTTTGAAACCGAATACCAGAAAG |
| | | TTATGATCTTAAAATAA |
| 323 | JW13146 | ATGAGTATAATGAACATAATTACGGTACTGAACCATATTCACCAGATTGCAGGTCTGAAACGAAATGAGTTAGG |
| | | TGATGATGATAACATCTATTCACATCCTGTCCTCAAATATATTGCTCCAGAGAACAATAGCACCACCCTGCCGGA |
| | | AAAACGGGACCTCATTAAGGAATTGCTTTTAGAATATCTCCCCTTTTTAGAGGCGATGAAAGACTGCTACAAAG |
| | | AAGTAAAAGTAGAGAAAGATAAACGCAACAATAAGTATGTGAAGGATAAAAAACAGGTATTCATCCGGAATTA |
| | | TAGCGACATCTATTGTATCTATTGGACGCTGTACAACATTTTTAGTACCTTGATCAATTACCGCGATAAGAGTTG |
| | | TCATTATCGTTTTATTCCGTCTAAAGATCAAGAAGAGTATTCCAAAAGAACAGAATATGATACAGTTCGGATTAT |
| | | TAACAAATACTATACGTACGCCCTTCGCAAAACGAAAGAGAAATATGGTTATACCACCGAACAGTTGAGCTTCA |
| | | TACAAAATAACAGATACACGGACGATAAGAAAGTGAACTTTAATTTCTTTCTTTCCATGCAGTCTTCGAACAATA |
| | | ACCTGTCACATGTGGGTGTAGCCCAGTTAATATGCCTGTTTCTCCAAAAAAAGGACGTGGACAATTTTTTAGAAA |
| | | ACCTGCCTATCTATGACAATCGCTATTGTCTGGGGATTGATGAACACATGATTATTAAACGTTCACTGGCGCTGC |
| | | ATTCTATTAAACTTCCTAAAGAGCGTCTCAACTCAGAAAAAAAGGATATGGCGATTGCAATGGATATGCTGAAC |
| | | GAACTGAAGCGCTGTCCTAAAGAGCTGTTCAATACTCTGCCTTTCGATAAACAGGAACGTTTCCGCATTATTAGC |
| | | TCGGATCATAAAGAAGTCTTGTTGATGCGGAGCACCGATCGGTTCGTGCCCCTGATTTTGCAATATATTGACTGC |
| | | AACAAAAAGTTCAAAGACATCAGATTCCACGTCAATATGGGCAAACTTAGATACCTTTTTTCACCGAATAAACT |
| | | GTGCATAGATAATCAGGAACGTGTTCGCATGTTAGAGCATCTTCTTAACGGGTATGGCCGTATAGACGAGGTTG |
| | | AATATCTGCGTAAAGCGCCGAATGGTACTTTTGGAAATACAAATATTCCAATTAAAGGTTTTGGCGAGATTGAG |
| | | CGGGATAGCGCAGACTCTAATAATTATCCATACATCGTTGATACACATACGCACTACATCTTAAACAACAATAA |
| | | GGTTGAATTTTGCTTTTGTGAGGACAGCAAAAATAAAAACGTATTTCCAACTATAGAAAAAGAGATTAACGGCA |
| | | AATGGTATGTGGAAAAAACCGCCCCTCATTGCCGAATTAGTTTTTTTGAGATACCCGCTATGGCCTTCCACATGT |
| | | TTCTGTGTGGGTCCGAAAAAACCGAAAAACTGATCAAAGACGTGTATAACAATTATATCTCGTTGTTCAACGCTT |
| | | TAAAGAACGGAGCATTGACAGAAGAGAATTATAAAGATTTTAATATCCCGGAGTGCGACATGCCCAAAAAAAT |
| | | CCTCGATTTTCTGAAGGGTAAGGCGGAAGGAAAAAGTATCGACGGATACGTTAAAAACATTCTGAAGAAGGAG |
| | | TTAAAAGACACGAGAAAACGCATCGAACAAGCACCATCGGAAAAAGATCAGATGCCGTCTAAAAGAAATAAAA |
| | | TTGGGACCAAAAGTTATAAACGCGTTCTCACTGGAAAACTGGCTGATTTTCTGGCCAAAGATATTGTTAAGTATC |
| | | AGCCTACACTGTGTGCGGGCGATGATTATGGGATTGATCGTATGACAGGTATGAATTATCGTATTATGCAGGCA |
| | | ACTATTGCTACCTACAATGACGACGATGACTTTGATACATTTTACAATGTTTTTAAAATGGCCGGCTTAGTCGAT |
| | | GGAGATGAACGAAAAAATCATCCTTTCTTAAAAAAAGTGTTGCTGAGTAAACCCCAGAATACAGTGGCCTTCAA |
| | | TATAAAGTACCTGAAAGAGAAGGAAATCTATCTTCGAAACATCGAAAAAAAGAACATCATTCCGCCGTTCGTCA |
| | | ATCGTGAAAGAAATAAGTGGTGTGAACGCGATTGTGAGTATTATCGCATTTTAGGTGAAAATCTTTTAGACCTG |
| | | CCAATAGAACTGCCCCGTCAAATGTTTTACAAAGATATTCGGGCGCAACTGGAACCTTTAATGCCAGAAATAGA |
| | | TTTTTCTCAGACCAATACAACTTACCTGATAGCTGAATATCTTAAACGTGTACGTGGCGATGATTTCCAGGAATT |
| | | TTATTCATGGGAACGGAACTATCGCTACATTGATTTTCTGAAATGCATTCGCGATGATAATGGTCGTATCTGCGC |
| | | ACAGTATACTACAACAGAAGAACGCGAATCACTTTGGGAAGATTCTAAAAATCGTACGGCAGAATATAAACGG |
| | | AGCGAGGAACAAAAAAACGAGAAGAAAGATAAAAATTTTGAAGATTCCCTCGAGGAGCGTATCAGCTCATCCC |
| | | ATATAAAGTTTCAGAATACCGAAAAGCATATACGACGTTATAAAGTCCAAGATGCCTTATTATTCATGATCGCC |
| | | GAAAAACAGTTTGGTAAGTACCTTGATGATATTAAGGCGAAAAATTTCAAACTGAAAGATATTATGCCGGATAC |
| | | CGACAAAGGCATCTTATCCGAAACTATGCCGATTGACTTTACGTTCACCAAGAAAAACATCAAATACAATATAC |
| | | ACGCAAAAAACTTGAAATTCAAGGAGTACGGCGACTTGTTTATCTTAGCTAATGATAAACGTTTGGAATCTCTGT |
| | | GGAAGATTTTGGCCACCGATACGGTGAATAAGGAAGAAATCAGTGAGGAATTTGCGAAATATGATTTTTGTCGC |
| | | CCGGATATAACTAAGCTCGTTCTGGACTTCGAGAAGGATGTCCTCGAAAAATACCCGGAGATCATTGAAGAGGC |
| | | TGAGAAAGAATATAAGAAATTTGACTTCAAGTTTATTCTCGACTGGCTTATTAACAAGAAAAAATTAAACTACG |
| | | GGATCCGTAACAAACTTCTGCTGATACGAAATGCCTTTAATCACAATTCGTATCCAGACAAAAAGTACGCAGAG |
| | | GTTAGAGAACTGCCGGAAATAGCTACTAATCTGAAAAAAACTTTTGACGAGAATGCGAAGAAGTATGAATAA |
| 324 | JW13147 | ATGTTTGATAATGAACAGAAAAACCTCGAAAAAGAACCTTATTGGGGCGTTTTTCTTAACCAAGCGCGCCTCAA |
| | | CGCCTATATTGCTTTGCGTGATATTAGCGAACGCTTGGAAGAAAACGCGGCCGATGAGGATTCTCTGTCCGAGT |
| | | GGCCTGTATTAAAATATCTTGATAATGATACTGACGCAGTGAAAAGTCGCCGCATATTCGACCTTGTTGAAAAA |
| | | CACTTCAGCATGTTAAAAATTATCTATGGTGGCGAAAAAGAGGGAGATCTGGTCAAACGATCCAAAGAGTACA |
| | | AGATCATACTCAAGTGCTTATTCCGCGCGCTGAACTTCTACAGAAATAAGTTTTGCCACATGTATAGCGGTAATC |
| | | GCGCACGGAAGTACAACGAGAAAGAACTGATCAAGTATCTTGAGGATTGTTTTGATGCCTCGGTCCGTAAAATT |
| | | AAAGAGTTACGACGTTTAGATGAAAAAGACGTACTGCATCTTCGGCGGAAAATTGCCGAAGGTAAAGATGCCA |
| | | ACAAACGCGTTATTGACAACCCGCAGTTTCGCTATCCCTTCAAAAATGAAAAGGGTGAACTGAACGAGAAAGG |
| | | ATTGTACTTTCTGGCGTCAATTTTCTTAGACAAGAAAGAGGCTCATGAATTCTTGAAGAAGCAAGAATACTTTAA |
| | | GAACGACTCGGAACCTAAGTACCGCGCCACTCTGGAATCGTTTTATCATTATCGTATTAAACTTCCACGGCCGGT |
| | | TATCGAAAGCGATGTAGACAAAAATGGGTTAGCTTTGGATATGCTGAACGAATTAAAAAAGTGTCCAAAGGAA |
| | | CTCTTTGATTTGTTGTCTAAAGAGCAGCAGGAAAAATTCCGCGTTGTCGATAGTGAAGATGCGGACGAAGAAGG |
| | | GAACGAAATCTTAATGCGGCGTTATTCTGATAGATTTCCCTATCTTGCGCTGCGGTATTGTGATGAAAATCAGGT |
| | | CTTTGAACGTATTCGTTTCCAAATCGATCTGGGTCGGTATTACTTTAAGTTTTATCCCAAAGAAACTATTGATGG |
| | | CAAAACGCAACAACGTTCCCTCGATAAACGTTTAAAAATTTTCGGGCGCATCAAGGATGTAAAAAGTAAAGTGG |
| | | AACAAGAATGGTCAGGCATAATTAAAAGTCCAGATACAATAGAAGAGAATCCGAATGAACCATATAAGCTGAA |
| | | AACCACGCCGAGATACAATATTGTAGACAATCAGATAGGATTTGTAATTACCGGCGATAAAAACCTGCCGGACG |
| | | TAAAACGTCCTGATGGTCGCATTGAACTGGAAAAACCGGACGGGTGGCTGTCCATCTATGAACTGCCTGGCATG |
| | | TTATTTCATGGTTTGAAGTATGGTTTCGACAAGACCGAACGTATGATAAAGATCTACATAGAAAAGCAGCGTAA |
| | | AATTTGCAAAGAGATATGCGAGAAAGGTACTATTACTCCAGATGATGGCGAATCCATGCCGGAAGCCTTAAAAG |
| | | GCGGTGCCAAAGCAGCAAAACGTAATTACAGCGAAAAAAAATTAGAGCGTATGTTACAAGATACGGAACAACG |
| | | GATTCGAGCCATACAGACGACACAGAAACGAATGGATGAGCCTGGGAATAAACCAGGCAAAAAAAAGTTTTTT |
| | | GATATACGTGCCGGCAAACTTGCTGATTTTCTGGCTCGCGATATTATGGCGCTGCAACGGTTTGATCCGGCTAAA |
| | | CACGGGAAAGACAAACTTACGGCTATTAATTTCCAGGTGCTTCAGGCGACTCTGGCCTTTTATGGCGCAAAGAA |
| | | AGACGTAATTGAAGATATGTTTAAAGGTATTGGTTTGCTCGAGGGTGATAATCCCCATCCGTTTCTGAATCAGAT |
| | | TGATCCGGCACAATATAACTCAATTGCAGGGTTTTATCAGGCGTATTTGCAGAAAAAGCGTAGTTATTTGGAAG |
| | | ATTACCGCAAAGAAGAGGAATATGACGAGCAGTTTTTACGTCCGAAAAGACAGAGATATGCCCAAGAAAAAAG |
| | | AGAGATCAAAACCGTTGCTCGCCAGCTCCTGGACAACCCCGTCAATGTTCCAAAAAATTTCTTCAAGAAAGAGA |
| | | TAGAGGAATTTGTGTTTAGTCAGGACCCAAGCCTGAAGAAATCTAAAATGAATACTGCGTACATGATCCAGGCA |
| | | CTCTTTGAGAAACACTATGGTCGTCAACAACCGTTCTATTCGTATAATCGAACGTATCCCGTTGTGTCCAAAGCA |
| | | ATCGAGTATGGAAAAAAGGGAAAGAATAAGAAAATCGCGAAGGTCTTGATGGCAATCGAGCCTAAGCTTAATT |
| | | ACATGGAAATCAAAAAAATCGTGAATGAAATGCCGGATGGGCAATATGAACCTGAGAATCTTAAACGTAACCT |
| | | TTATGAAGGATATAAAGACTACGAAAAAGATGAAAGAATCATTCGTCGGTGTAAAGTTCAGGATGTTGTCAGCT |
| | | TTATGATGGTGGAAGAAACCTTAAAAGACCAGCTGGATTTTAATGGGAACGTGCTGACCTTAGAGAAGATTACC |
| | | CCCTGGGAAGCGTCTCCGTTCAAAAAACCAGTTCTGTGCCATACAATAATTTCAATTCCGTTCAACACCAAAGGC |
| | | GGTCATACGGATAAAGATTATGTTGATTTTATCAAGAACAATTTCGAAGGCAGTTACGATTGTGAGCCGAATAA |
| | | AATCATACTGAAGTACAAAGTGACAAGCAAAGACACTAAACTCAAAGATATCGGTAAGTATCGTATGTATTCAC |
| | | ATGATCGTAGACTGCCAGGTCTGCTGATATGGAAATACCGCCCAAATGATCAGAATGGCAATGAGATAAAGTTT |
| | | ACAGAGATCGAGCAGGAAATTAAAGCATTCGAACGGCGCCGAATAGAGATCGCTCAGTGTCTGTACACACTGG |
| | | AAAAAAAAGTTATTGACTCTTGGTTCACACAAGACGAGCTGGGAGAAGAGCACATTCCCTTTAACAAAGTGATT |
| | | GACGTCATTAAGGCAAAAATGCCTAATTTTGAAGACAAATGCAACGTGTTACTGAAGATTAGAAACGCAATTAA |
| | | TCATAACCAGTTTCCTGTGTACGAACAGGCCATCCAGACAGCTCCTGGCAAAGAGATTGCTGGAAAAATGTTAC |
| | | GCATCACCGAGTCATATATCGAACAGATAATGGCAAAAATTGACCCGGACTTTGGACGAACCGAGGATGCGGA |
| | | ATCTTCACGTTAA |
| 325 | JW13148 | ATGATCAAAAAACCGGCTAATCGTTTTGCATTACCCAAAGTCATAATTAGCGAAGTCGATTCGGAAAACATCTT |
| | | GGAGTTCAAAGTTAAGTATGAAAAACTTGCTCGGCTTGATCGGTTTGAAGTCGAAGCAATGCATTATGATGAGA |
| | | AAAACGAAATAATTTTTGACGAGGTGAAAGTGAATGACGGCTTAATCGAAATCGTTTACAAGGAGAATAACAA |
| | | AAAGTTAGTGGTGGAGATTGAAGGCAAAGCTTATGAAATCTCAGGTCAAAAAATAGGCGGCAAACAGAGATTG |
| | | TTAGAGAATCGTGTGAGTAAAACAAAAGTGCAATTAGAGCTGATGAATCATATTGAAGATGGAGAGGGGAACA |
| | | AACGGGTGTCACGTACAGAAAGAGAGTTGATTGTTTTGGGTGATATTAAGCTTTATTCCCAGATTGTAGGGCGC |
| | | GAAGTTAAGACAACTAAAGAGATCTATCTGATTAAACGTTTTCTGGAGTACCGTTCTGACTTGCTCTTTTATTAT |
| | | GGTTTTGTTGATAACTTTTTTAAAGTCGTTGGGAACGAAAAAGAACTGTGGAAAATCGATTTTAATACATCTCAC |
| | | AGTGAACAGCTGATTGAGTACATTAAGTATACCTTTAACGACAACCTGAAGAATGATCGTCAGTATCTTAATGA |
| | | ATACGTGACTAATGACGTGCAGATTCAGAAAGATTTTAACAAGGTAAAAAATGTTTTCTCCGAGTTGCGCCACG |
| | | CACTGCTGCATTTTAATTACGATTTTTTCCAGAAACTGTTCAACGGCCAGGATGTCGGGTTTGACCTTGATATCG |
| | | AATTTCTCAACCTTTTCATAGATAACATCGATAAGCTTAACATAGAGACGAAAAAAGAGTTCATAGGAGATGAA |
| | | AAAATTCGACTGTTTGGCGAAAATCTTTCACTGGCGAAAGTGTATGAACTTTATTCTCACATTTGCGTGAACCGT |
| | | GTTGGGTTCAACAAGTTTATCAATAGCATGTTAATTAAGGACGGAATTGAAAATGAGGCTCTGAAAGATAAGTT |
| | | TAGCACCAAATTCACCGGTAATGCCTATTATATTGATATTCATAATAGTCGGGAGTATAAAGACCTCTATAAGA |
| | | AGCACAAACGTCTGGTGATTAATGTTAGTACCCTTAAAGATGGCAGAGCAATTCGCCAGGAAAATAAAAAAATC |
| | | GCCGAACTGAAGAAACAAATGACCACTATGACGACTACCAACAGCCTGGCGCGCCTGGAGTGTAAACTTCGGTT |
| | | GGCCTTCGGCTTCCTGTACAATGAATATAATGACTACAAAGCGTTTAAGAATTCCTTTGACTCCAACTTGAAAAA |
| | | CAACCAGTTTGACGTTTCTAATGTAGATACGTCAAAGGCATATTTCCTGTCTACTTATGAAAACAGAAAACCACG |
| | | AACCCGTGAACGAATTGATAAAGTTTTAAAAGAGATTGAATCGCTGGAGCTGAAAGGACTGATTGCAAATGATC |
| | | CATTACTCAAGTTTATACTCCTGATGTTTGTCTTCATGCCCCAGGCCCTGAAAGGTGAATTTCTGGGCTTTGTTAA |
| | | AAAATACTATCATGATGTCCACAGTATCGAGAAGGACACAAAAGACAAAGAAGAGGATCTGATAGAATTCATG |
| | | CCGCTGAGCCTCAAATTAAAAATCCTGGGACGCAATATACGCACATTAACTCTTTTCAAATATGCCTTAGGATCA |
| | | CCTATCAATTACAATTCAACTGATGAAGTATTCTATGTAGAAGGAAATCGTTATGGAAAAGTCTATAAACAATT |
| | | GGGTATAAGCCATAACCAAGAGGAATTCGATAAAACATTGACGGTTTCCTTATTACGGTACTACTCAGCACTCTT |
| | | CAAACTGGTCAATGATTTTGAAATCTATTCTTTAGCGAAAGGTAATCCTACAGTGGTTAATCTTGATGTGCTGGT |
| | | AGACGATTCGTCTAGTCCGTATAAGTTTCGCGGTTTTTACAATTTTGGTAATATGATCGGGCAAATACATGGGCT |
| | | CACGCATAACCAGATCAAAAACGAAGAGATCATGATAAGAAACAAAATTGCCCACTTTGACATAGAAATGTTG |
| | | CTGAAAAAGCCACTGTTAGCGCAAATTAAACTTGATGTGCAGCGCAAAAATCTCATTAAGTTCATTGAAGACCG |
| | | CGGTGATATGAAATCCGTATTAGGCTATGATGCTATTAATGACTTTAGCATGAAAATGGTGCATCTGCGTACGA |
| | | AAATCAAAGTAAACAGTGATAAGCTTCAGATAATGATGGGCCTGCTGACCAATGTTAAAACCCCGAACGACTTT |
| | | TACAATATCTACAAAGTAAAGGGTATTGAAAGCATTAATCAACATCTTTTGGATGTTATTGGTGTCACGGAATCG |
| | | GAAAAGAAGATTGAAAAAGAAATCAACGAAGGTAATAAACGAGCGGTAAAGTAA |
| 326 | JW13149 | ATGATGGAAACCATTAAACAGGAAATAGGTAAAGAATACAATCCTTTTAAGACCGAGGATAAGCATTATTTCGG |
| | | CGGCTTTTTTAATCTGGCCGATAATAATATTAAGGAAGCGTTTAATGAGGTTTCGATTCGGCTTAGTCAGAAAGG |
| | | TGCCTCTCCCAAAACATTAATTGAAAAATACACCAAGGAAAATGTCAGTCTTGTAGACTATGAGCGCTTTGTTAT |
| | | ATTATTGTCTGATTATTTTCCAATAATCAAGGAGATCGACCAGATTAATAAAGATGAAAAAGATAATAAAGGGA |
| | | TCCTGATACCCAAAACAAAAAATGACCGTATTAAAGATTTTGAAGTAATTCTTGTCTCCATGATCGATGCGATAG |
| | | AAAATCTTCGTAATTATTATACACATTTCTATCACGAATCCATCGAGATTGATAATAAGATGTTCTATTTTCTCGA |
| | | TAAAGTGCTCTTTAAAACCGTGTTGGACACGAAACGAAATTACTTGAAAGAAGATAAAACAAAAGAGATGATA |
| | | AAGAACACGCTGAAAGAGGATTTTAACAAGATCTTTAACCTTAAAGTTGACGAGTATTTGACCAAAGACAACCT |
| | | GACGTCAAAAAAAATAAAAAAGGCTCGGAAGTATGGTAATAGCTTTGATATAGGTTTGACTAAAGAAATTACTG |
| | | CGAGTATCTACAATGATGCCATCAAGTATTTCACCTATGATAAAAACGAAAAATCCATTCTGTCAGATAGTCGC |
| | | AAGACTGCGTTTAATGAACGCGATTATTTTTATCGTGATAAGAATTTCGATCTGCCAATTAGCTCTAGCGGCATT |
| | | ATATTTTTGCTGAGCTGTTTTCTGAACCGTAAAGAAATCGAAGACTTGAAAAGTAATATTAAAGGTTACAAAGG |
| | | GAAGGTTACTAAAAATGAAGAACCAACATTAGAAAAGAATTCCATTCGCTTTATGACTACTCACCGCATCTATT |
| | | CAATATGGCACTATAAAGGACTGAAAAATAAGATTAAAACGAGCGAAAGCGCCACAAAAGAAACACTGTTGAT |
| | | GCAAATGATTGACGAAGTATCAAAAGTGCCTAACGTGGTTTATCAAAACCTGAGCAAAGACCTGAAAGACTCCT |
| | | TTATCGAAGATTGGAACGAGTATTACAAACATAATGAGGAGAATACCGAGAACACGATTAATAGCAAAGTCAT |
| | | CCACTCTATTGTGCGTCGCCGGTACAAGGATAAGTTCAACTATTTTGCTATTCGGTTCTTAGACGAATTTGTGAA |
| | | TTTTCCGAGTTTACGTTTTCAGGTGTACCTGGGAAACTATCTTGTTGATTCCCGTCCTAAAGAAAGTGCAAACGT |
| | | CATTACTGAGAGAGAGATTAAGAAAAAAATCTTCGTCTTCGGTAAACTGAACGAAGTTAATCAGCTGAAAAATG |
| | | ACTTTTTCAGTAAAATTGAGGAAGATAAACTGGAAACCAATCTTGAAGTGTTTCCGAATCCGTCTTACCATTTTC |
| | | CAATGGAAAACTCGGAAGAGCTCAAATCGGCGAACAAGATTGGAATCTATCTTTCTGACATAGAAAACTTGCTC |
| | | GAACCGTTTAAGAAACATCTTAACAAAAATACCTCACAAGAGAAAATTTCGCTGATCGAAGACATTACCCAACA |
| | | GAATGAAAATATCAAAATTGGCCAACCGTTAGCCTACCTTAGCATGAATGATATACACAGTGTAATCTTTGAAG |
| | | TGCTTAAAAATCCGATTATCAAAGATGGCAAAATAGACGGTAAGTCGGTCGAGAAAAAAATAAAGAACCAGAT |
| | | CTATAAACAGATAAACGAGATCAAGAACAAAAAGGAAGAGAGCAAAATTATCAAAAACTTTCGTAAAAAAACC |
| | | GAAAGCGACTTGGACCTTAAAAAGATGATTGATGATCTGATCAAAGAAATAAAAATCACTGACGACAAGATCA |
| | | AAAAACTGGACGAAAACAAAAAAGAGTACTTTGACTATCAAAAAAACAAAACCGCCAAAAAACGCAATTACAT |
| | | ATTACAGAATTCTGAAAAAGGCGAAATTGCAACCTGGCTCGCTAATGATATTAAACGATTCTTTCCAAAAGAGT |
| | | TCAAAGAAAAATGGAAAGGCTACCACCATCGCGAATTACAGCTGTCAATCGCGTATTACGATACGCAGAAACA |
| | | AAATGTCAAACTGCTCTTGTATGGATTAGATTATCGCAAAGAGATCCCGATGCTGGATTTCTCAAAGCCCAACTT |
| | | TTTAGATTTTTACGAGTATTATCTGAAAAAACGAAAGCCGCAATTATACAATCTTTTATCGGATCTTGAAAAATT |
| | | AGAGCGCGGCGAATCCATTAATAAGGAAATGCTGCTGTCTAAGTACTTTAAACTGTTCCGCAAGAGTAATTATC |
| | | AGAACAAAAGCTTAGATGATAAAATTAATACCGCGCTGGCAAATCCTATTTTTATCGAACGTGGTTTTATGGAT |
| | | GGTAATCCAACAGTAATACCGAATACAACAATTAAAGGGAATGAATCGAGATTTGCAAATTGGTTCGTGGCTTA |
| | | CAAACAGTTTGATGAATACCAGAAGTTTTACAATACCGAAGAATATTTCCTCGAGTTAAAGGACAAAACCCCTA |
| | | ATGAAATTAAAAAAATTCAATCACAGATCTACACGCAGAAAAAGAACGACTGGGCAATTTGGAAGATGATCTC |
| | | ATATATTTTTAAAGACATCTTCAACCAAGATCTTCAAAACGTGTCACTGTCTGAGCTTTATCAGAATCGAGAACA |
| | | AAGATTGGAGAATGAGAAAAAATCTAAAGAAGGAGAGCGTAAGCAGAACTTCTTTTGGAATAAAACGGTTGAT |
| | | TTGCAGCTGAACAACAAAATTAATATACCAGAGGTTAAACTGAAGGATATCGGCAATTTCCGCAAGTATGAGAA |
| | | AGATGAGCGGATTAAAGTTTTTCTGTCATATAACGATATTACGGATTGGATGGCTTATCTGCCTAATGATTGGCA |
| | | AGAGAAACATACGATTAAGCCGATAAACGTAATAGATATTCAGATTGACAAGTACGAAAAAATCAGACAGCAT |
| | | CATCTTTTAAAGGAAGTTCAGAATCTTGAAAGCGAAATCTATAACAAGGCTATTAACAAAGAAGAATTACTGCA |
| | | GGAAGATATTAAGGGGAATAAAAACCCCAACTTCAAAAAGTATATAGTGAATGGGCTTCTCAAACAGATCAAA |
| | | GGTCTGAACGTTGAAAACTTCAAAATTCCGAACAGTGATAAAGAGTTTAATGCACTGTCTGAAGAGACATTGAG |
| | | ATCCTATACTGAATTAGAACAGAAGGTTACTCTCCTGGTCCTCATTCGTAATAAGTTCGCACATAATCAGCTGCC |
| | | TAATAAAGATGTATATGCATTCACACAATCCATACGGAAGCGTCAGAAAAACGAAACGTATGCCGAATATTATT |
| | | TCAACCTGTTTGAGAAAATTAGAGGAGAGATTTGA |
| 327 | JW13150 | ATGGAAGAGAATAAAGTCCGCTTTACAAACACGTCCAACGTTAAACGCCCGAAGAGTCTGCTGAAACAAATGG |
| | | GGCACAAAAGTACATTTCTGCTGGATGATACTTTAATTGTAACTTCTGTGGCTAAAGGTAAAAATGAAGATCAA |
| | | AAGGGTAAAGAACAAGCCGCATCCAAAAGTAAAGTTGCTCCGCAGTCACATATTGAGTTGTTAAGTACTATCGA |
| | | TGGAGAAGTACTCGGCGGTCGTAAAGAATTATTAGCTACAACGGTTAGCCGCCAAGAAGTTACGATTTCCGGAA |
| | | AAATCTACAATCTGAATACCAAAACTCCGGAAAGCACAACCGTAACCATGGCGAATCCTGCTGTTCTGTTACAG |
| | | AAAAATAGTAGCACTACTCAAGTTGCGAGAAAAACCGATTATCTTGGCATCAAAAGTGCTTTAGAGAAGGAGTT |
| | | TTTTAACGATTTTTCAAACTCCGGTTTTAACGACAACGTTCGCGTTCAAATCGCTTACAACATTCTGGATATACG |
| | | GAAAATCTTGGCAACAACCATCAATAATATAGTTAGCATGTTTTACAATCTTGATAGAACCACAGGTGACATTG |
| | | CAGATGAGACGAAGAAAAGCACAAATACCGACATTATCGGCATGATTAAAACATATCTCAAATATGACCAGGC |
| | | AAAAAACTCAAATGACCCATCTATTAAAAAAACCTTTAACAAAGCAGAAGAGCTGCTGATTAACACAAAAGCG |
| | | TATCACACCTATTTTGACCGATTATTCACGACCCCTACTCGTCAGGCTTTTAAAGCCGGTAAACTTAAAGATACA |
| | | AACGATAAAAAATCAAACCTTCCATGTGAATTCTCTAAACTTAGCGAAAAGGACCAAGAGGAATTCAAAAATG |
| | | AAGCGATTACGCATAATTATGACGTTCTGCGATTACTCGGCTTGGGTCGTCAGCTTGTGGTGCATGAAAAATACA |
| | | AGTCTGACGAACACAATAAAGATAATTCGATCGAGTCCCTGTTCAACATTGACAAGATTATAAGAAACAGCAAT |
| | | GATACCATGGACCTTCTGAAACGGCTGGATAATATCTACCTGGAATCGGCCGAACAGATTGATAACAGCTTTAA |
| | | GAAAAGCTCAGCGACAAATTGCTTAATACTCGAAGAGCTTTACAAACGCATTGGAAAAGCAAAAACACAGGCT |
| | | GAACTGTACAAGGATTATTATCGTTATGCCATTTTAAAAGAAGACCAGTGCCTGGGAATCTCGTTTAATCGTATT |
| | | AAAGAAGCGGTATTGGAGAAGCTCAAAGAAGAACAGCGCTATAAGAATTTCGAGTATCGGAATGATAACAAAC |
| | | GGTTCTACACCTTGTGTAACTTCGTACTGTGGCATTATATGGTCGATCTTCGGGACAGAAATGAGACCCGCGCAG |
| | | GACATCAGCAAACGGATAGAAATGAAATAGAGGAGCTGAAGTCTAAACTTCGCGCGTGTTGCGATAATAACGG |
| | | CGATGGCGGTGAAAACCAGAAATATGCCATCTACAAAGAATTCGTTAATTTTCACTGGACGGACTTGCGTACTA |
| | | GTATCAATACGTTGTATGATCTGTGCGTCAAATATAAGTATACTGCAAAAAATTATCGTATAGACTCCAAGATTC |
| | | AGATCGACAATCAGTTATCTAGCTCAGCAAACAATGTCAAGACCTTTGTCAAGTACATCTATTTTTTATCCGAAT |
| | | TTCTCGACGGGAAGGAAGTGAACGTTTTCTTCAACGAATTGGTGGAGAAATTTTCGAATATTGCAGAAATTGTG |
| | | ACCACGGTGAACGACAATATTAATAATTCGTCAAAGAAGTCACCCAATATCTCTTTTCTCCCGCAGTATTCATTG |
| | | TTTCAGGAAGCTGGTACCGTAGCGGATCAGTTACGTAATGCTCGCAATATTAAACATGCGACTCTGCGTGCCCG |
| | | CATTAATGATAAAGTTGCGGATAAAGATAGCAAAATGTCTCCGTCGAAGCAAACAATATCAGATGCTCTGTACA |
| | | TGCTGATTGACGAGAACGATACCATTGAAAATTATAAACAGCTGATATGGGACGAGAAAGAATCTGGCACCTG |
| | | GGTGAAGAAGGCTACTGGTAATAATAAGGTGCGCAACTTCATCATCAATAACATAATTATGAGCAAATGGTTCA |
| | | TGTACATCTCGAAATATTCAGACCCTCGCAACTGTAGCAAAATTATGCATAATAAAGAGGCAGTGGTTTTTGCTA |
| | | TGTCAAGTATTCCGGATGCCCAAATCGAGAAATATTATAAACGTGTGACGGCCAAAAGTGTACAGTATGGCAAC |
| | | AAAACCGCGGAAGAAATGCGTAGCATTGTAATAGATGCGTTAATGAATTTTTCCCTCAAGCCCGTTTTAACGGG |
| | | AGTTGCCAATAATGATTATTCCAATGAAAACCGGGAACGTGATATCGGGATTATTCAATTGTATCTTACGGTCGC |
| | | CTACATTGTGACAAAAAGTCTGGTGAACGTCAACAAACGATTTTTACTGGCATTCTCTTGTCTGGAGCGGGACGT |
| | | TCCACTGATTTTAAACGGGAAGAGTTTTAAAGATGCCAACATTTTAGTTTTGAGCGAAAAGTTTATAGCAGAGG |
| | | ATCAGGAGATAGAACAAAAATATAAGCTCGCCAAAAAAAATCGAATTACGCCTAATATGTCAAAAGAAGACGT |
| | | CAAAAATGTGTTTAAAGAGTTGAACGCAGAATATAAGTCTAAACATTACGCAGAATCATTAACACGTCCACGTG |
| | | ATCCCGATTCCAAAAAAGATAAAAGTTGCGCGTACCTTGTGAATAACCTGAACGAAGCCAAGAAAATTGAGAA |
| | | CGTCCTGATGGATTTTAAGAACAAAGTCGATCACCTGAATGTAGTAAAAAGTGCGCCGGAATATATGTCGAATA |
| | | TCAAGAATATCGATAGTTTCTATGGCATCTTCTGTTTCGCGCTGCAGAACATACTGGCCGATAAAATGATAAAGT |
| | | GGTCTGTCAATGAAAAAAATTCTAAGGAAGATGTGGAGATAATCAATTCGTATGGGCAGGAACTTAAGAAGAA |
| | | ACTTGATTCCCGCCATACTTTTGATAGAGATGCAATGTGGATAATTAATATGCCTTTTGCCTATAATTTTCCACGA |
| | | TACAAAAACCTGAGCTCTGAGTATCTGTTCTACGACAAAGAATACGAGAAAACTAAAAAATAA |
| 328 | JW13153 | ATGAAAATACCTCAGATTATTGAGAATAACAAACATCTGTTTGGGGCGTACTCTACAATGGCGTTGTCAAATAT |
| | | CCGTAATATTCTGGATCATATAGCTACACTGGCCGATATCGAAAATGACTTCTACTCAGACTCCGACTCATATTG |
| | | GTACCATCCATGTATTAAAATTATTGATCCACGTAACAATTGCACAGACGCAGCTAAAGCCGACTTCGTGATTG |
| | | AAAAAATTAAGAGTCACTTCCCTTTTGTCCCTATCGTAGCGGAAGCCCAACGCCAAAGCGATATCTTACGGGCT |
| | | AAAAAACAGCTGGATATTGCCGTGAGAAATCGTGACTATCAGAAGCAAGAGGAACTGAAACACAGACAAAAAT |
| | | CATTACTTTCCGTTACGAATGCAGATATCTATCGTGTTTTAAACAACATGTTATGGGTCCTCGCAAACTACAGAA |
| | | ACCAAACCGTTCATTATTTGATAAGCGACATTAAGTTTGAAGATGGCTCCGATTTTCTGAACTATAATGAGCAAC |
| | | CGTTGTCGCGTATGCTTAATAATTATTTTACCATCGCCCTGCGCGATACTGCGAACAAGTATTCTTATTCTCCCGA |
| | | AGCTTTAAGCTTTATTCAGTCTTCTCGCTATAAGACCGAGAATCGCCGGAAAATCCTGGACACTGACTTTTTCCT |
| | | TAGTATCCAGCACAACAATAGTGATACAAATCCAAAAAATCTGCATCTGTCCGGCGTCGGTGTTGCTCTCTTGAT |
| | | CTGTCTTTTCTTAGAGAAGAAGTACGTTAATGTGTTTCTGCAGAAATTGCCCATCTATGGACCATATAAGAAACA |
| | | ATCTATGGAAGCTAACATTATTCGACAGTCTTTTAGCATCCATACCGTGAAACTTCCGAAAGAACGAATTGTTTC |
| | | GGAGAAATCAGATTTTACCATTGCTTTGGATATGCTGAACGAACTGAAACGGTGCCCGAAACAGCTGTTTTCGA |
| | | CGCTGTCGTATGCAGATCAGAATGCCTTTCGCATTGTGTCCTCAGATATGTCTGATGTGTTACAGGTGCGCCATA |
| | | CTGATCGCTTCGCCCAGTTATCTCTGGAATATATTGACCGACGGGAGCTGTTCAGCGATATCCGTTTTCACCTTA |
| | | ATATGGGTAAACTTCGATATCTTAAAACAGCGGATAAACATTGCATTGATGGTGTTAGCCGGGTGCGCGTTCTC |
| | | GAAGGTAAAATCAACGCGTTTGGTCGGATACATGAATTTGAAGCCCGTCGAAAAGAACAGGGATTCGTTGAATG |
| | | GCATGAACAGGCAGCGCGACTGATCAAGACTAACAAAAATATCGAGATCAGAGACTTTGAGCATGTAAAGCGA |
| | | GACGACGCAACTCCGGACAGCTATCCATACATCATTGACACGTACACCCATTATTTGTTGGAGAACAACAAGAT |
| | | AGAGATGCGTTTTGGCGATTACTTGCCAAAACTTGTTAAAGTGGACGATCAGAAATGGACGGTCTATAACGAAA |
| | | TGCCGACGTGCTGTATGTCGGCCTTAGAGCTTCCGGCGATGATGTTTCACATGATGTTATGTGGGTCCGAGGCTA |
| | | CAGAACATCTGATAAAGGCCGAAGTAGAGAAATATAAGAAACTCTTCGGTGCAATGGCAAATGGTACACTGAG |
| | | TAAAGAGAACATAGGTAATTTTGAGATTGCGATGGAAGATCTTCCGAAAAAAGTTATTGATTGCGTTAACCGCA |
| | | AGAAATCCGGAAAAAGTTTTGTCAAACAGATTAAAAAGGAGATTGCTGAGATGCTGGCCGATACTAATTTGCGT |
| | | ATTGTACGTCTGAAAAGTGACAAACGTAGCGTTGCTTCGACCCAAAATAAAATGGGGAAACGCGGGTTCAGAA |
| | | GCATTCAGCCTGGCAAACTTGCGGACTGGTTGGCGGCAGATATAGTCAAACATCAGCCAAGTTTATTCTCAGGC |
| | | GATGATTATGGCACCGATCGCATTACAGGGATGAACTATCGGGTGATGCAGTCAACTATTGCCACGTTTAATGC |
| | | GGCAACACCCGAACATTCGCTGGATGAACTCAAACGCGTTTTCAGCGCCGCACAATTAATACAGTGTCAGGAAC |
| | | GGGAACATCCATTTTTACAGAAGGCACTGAACCGTAACCCGCAGAATACAATCGATTTCTATGAATTTTATCTCC |
| | | AGGCGAAACGTGCCTACTACCAATCACTCCGCCAATCCATAGAGAATGGGGAAGATGTAAAACTGCCGTATCTT |
| | | AATCCGGATAGAAATAAGTGGGTCCGCCGTGGCGCCTCATTTTATTCTACCATGGGTGATATCTATCTGGAAGAC |
| | | ATGCCGATAGAGTTACCCCGTCAAATGTTCGACAAAGAAATTAAAAAAGCGCTGGCGCAACTCCCTTCAATGAA |
| | | GGGCGTGGACATGAAACAGTCTAATGTCACCTTCTTAATCGCAGAGTATCTTAAACGCGAACTCAAGGATGATT |
| | | TTCAGCCTTTCTACCAGTGGAATCGCAATTATCGGTTCACGGATATGATGATTTGTGAAGAGAATTGTGATACTA |
| | | AAGCATTGGCAAGCAAGTTTATTCCGCTCGAACTGCGTGAGGAGATTTGGGAAAAACGCGGTGAGCTTAAAGAC |
| | | GCATATAAACGTTGGGCTTTTGGAAGACTGAACAAAAATCCTGACATCAAACGTTTATCACCGGCGGAAAAAGA |
| | | TGAACTGCTGGAAAAGAGAATAGCGAAGTGCAGAAATGAATACCAGAAAAGTGAAAAAATCATTAGACGGTAC |
| | | AAAGTGCAAGACGTATTGATGTTCATGATGGTAAAAAGTATGTTTGCACAAGGAGTCTTTACTGCGGAATCTAA |
| | | AGAGTTTGCTCTGTCCACTATCACCCCGGATGCCGAACGTGGCATTTTAAGCGAAGTAGTTCCCATTGATTTCAA |
| | | GTTTAGTATCGACGGTAAGACGTATACGATTCACAGCAATGGCATGAAGATAAAAAACTACGGAGATTTTTACA |
| | | AGCTGATCAACGATAAGCGCATGAAGTCGCTCCTGAACATCGTAACACACTCTACCATTGAAAAAGACCTGTTG |
| | | GAGAAAGAATTTTCCACCTATGATGATAAAAGACCAGAAGCCGTGGAAATAATATTTGAATTCGAAAAAGCAG |
| | | CCTACGAAAAACACCCGGAACTGAAGGAACTGTTAACACCTGAACGTAAGTTTGATTTTCGTGCCCTGTTAGAA |
| | | GAGTTACAGGCTAAAAGTGATCTTAGCCAAATTGATGCTCGGTATTTGTCACAGATTCGCAATGCATTCAACCAC |
| | | AATGCTTATCCTCGGAATCTGAAAATAACGAGTAATATACCTGAGATAGCTCAGGAAATGGTGAACATTTTTCG |
| | | TATTACCGATCCCTACAAAAAAGGTAAAAAATAA |
| 329 | JW13154 | ATGGAATCTTTAATTCAGCAAGAAGAGAATAAAGCTAGAAATCTGATCGATGATCCACAGTATTTCGGGGGCTA |
| | | TCTGAACATGGCTCGACATAACATATTCTCAATCAACAATCACCTGGCGAGCTATTTCGGAACCTCTGGTTTGTC |
| | | AAATGAAGAACAAATTCCGGATAGCTTCTTGCTGTCAGCAGATAAACGGCGGAATCAAGAAACTAAAGAACTG |
| | | ATTAAAGAAAACCTGTTGTTTGAAAGAACGCTGCGCTTTATGCCGATTTTTAGAATTTTTGATACAGAAAGACTG |
| | | CCAAAAGAAGTTCGTCTGAACACCGAAAATATGGAGAAAGATTTTGATGAGATGCGAAAGTGCTTAAAACAGG |
| | | TCTTTAGCGAATTGAACATTTTTCGAAATGACTACACACACTATTTTTCCACCAACACCGAGACAACTCGCAAAC |
| | | TGCAAGTGACGCCCGAACTGGAAGAATTTTTGTCTCGCAGTTTTAAGTTGGCGATTGCCTTTACTAAACAGCGTA |
| | | TGCGTGACGTTTTAAAGGAAGAGGATTATAGCTTAGTCGAAAAAAAAGTCATGGTCTTGCCCGAGGAACGCGTT |
| | | ATTACCACTGAAGGCATTGTCTTTCTTGCGTGCATGTTTCTGGAACGTGAGTATGCGTTTCAGTTCATAGGGAAA |
| | | GTTGTGGGTTTAAAAGGTACGCATCGAAATGAATTCATTGCAACCCGAGAAGTTCTCATGGCTTATTGTGTGAA |
| | | ACTGCCTCATGACAAGTTTATAAGCGAAGATAGTAAACAGGCCCTGATATTAGACATCGCGAATGAGCTTAACA |
| | | AGTGTCCCTTGACATTATATGACGTGATTACTGAGGATGGTCGCAAACAATTCAGACCTGAATTAACGGACGAT |
| | | GCCAAGAAAAGTGTGATTGAAAATTGTATCAATGAGGACCTCGAGGTCGATTACGATGAATACATACAAATTCT |
| | | GACCAAAAAGGTACGCTATAAGAATCGCTTCCCATATTTTGCCTTCCAATACATAGAGGAAACGAAACTTCTGG |
| | | ATAACTTTCGCTTTTCCATTGACCTTGGCCAGTTTGTTCTGGCCGAATATGAAAAACGGATGGCGGGTGAACCAA |
| | | CCTCTCGTATTGTCGTAGAAAATGTAAAAGCTTTCAAGTATCTGAAAGAAGTGACAGATGAAGATGTAGTTCTG |
| | | GGACTCATGAAATCGAATCCGCCTAGCAATCGTTTTCTGCAGTTTTCACCTCACTACAATTCCGATAATAATAAA |
| | | ATTGGTATTAGTTCTAAACCACAGACAAGCGTAGTGAAAAGTAAAGGGAAAATAGACAGCAAAATCGAGTACA |
| | | AGATTAAACAGCCGTTACCGCGCGCATTTGTGTCTATACATGAATTACCGAAAATCATCTTGTTAGATTACCTCT |
| | | CCAAAGGGTCTGTTGTCAAGATCATTAACTCTTTCATCCGTACCAATAAAAGTATTATTCTGGATGTAGACAAGA |
| | | TCAAGGAGATCAAAGAAAAGCTCCCGAATAACGAGGTATGGTTTCCTTTTCGGCGGATGACCGATTCCAAAAAA |
| | | CACTCAGCCTACTCGGAGAAACAGAAAGCGGAAATTTTGCGCAGAAAAAAATTGCTGAACGAAATTCTGAGTTC |
| | | CTACTCGCTTAACATCAAGCAGATTCCCAGTAGAATTATCGATTATTGGCTGAACATTATTGATGTGCGCAAAGA |
| | | ACGTTCGTTTTCTGATCGTATCAAACTGATGAAACGGGATTGTGCCGATCGGTATAAAGCCGTGAAACGAAATG |
| | | AAAAAGATAAAACAGTGGCGATCCCTAAAATCGGAGAAATGGCAACGTTTTTGGCAAAAGATATCGTAGCTAT |
| | | GATCGTAGACGAAGACAAGAAATCTAAGATAACGTCTTTCTACTACGACAAAATGCAGGAGTGCTTAGCACTGT |
| | | ATGCGAATATAGAAAAGAAAAACCTTTTTATTCACCTGGTTACAAATGAACTCAAGCTGAACGAAAAGGGCGG |
| | | ACATCCCTTTTTAAAACGTATTAACCTTAACAGTATAACCAGAACGAAAGAGCTCTATGAAATCTATCTTAGCGA |
| | | GAAGGCATACAAACTTAATTCCAAGGAAAATGATGAAAGCTGGATGAGTATGACTTTTTACCATCTTGAATGGA |
| | | ATGAAAAAGCAAAGAAGAAGATGACCAAGGTTGTCTATCCCGTGAATGACGAACAGATCCCATATACTATTAA |
| | | ACAGTGGAACAAACCGCAACAGCCTCTGTTTGACCGCTTTACGAATGAACAAGGAAAAAGTTCAGACTGGGTTC |
| | | GCCTGAAACCGCTGGATCTTCCGACTAATCTTTTCGATGAGGAGATTTGTGTTCAGCTGCGGTCCCGGCTTCAAG |
| | | AGGCAAAGATACCGTTCGACAACATCTCAAATTACTCAGAATTATTAAAACTTTGGTGGAAATCCCGCGATGAT |
| | | AGCACTCAGCCTTTTTATCTCCTCGAGCGCCAGTACAACATCTATGACGAGTCAGTCCGTTTTACATTGAACTCG |
| | | AAAAATAAGTTCAAAGACTATTATCAGGCCGCGCTGAAAAAAGTATTCAATCAGAAAAAAGCTGAACGTGAAG |
| | | CCAAACGTCGTTTTAATAAACGGCTGCCAGATATTCAGTTCAAAGATGTTGAACGTGTTTTCAAACATGCAATAG |
| | | CAGAAACTGAGAAGCAAATTAGAATTATACAAGAAGAGGATCGCATCATGTTACTGATGCTCGAGAAACTCAT |
| | | GGACGATCAACAGCTGAAACTGGGCAAAGCTGAGACACTCTTATCTTCAACCGTACCGGTTAAACAGAAGATTG |
| | | GTGATAAAACAAATTCGTCACGTGTGATCGTTGCTCAACGTAAACAAAAACATTATACGGTGTTACGTAAGTAT |
| | | CAGTATGATCGTCGCCTGCCAGAGTTATTCAAATATACCACCGAGAAAGAAGAGTTGAAGCTTGAGTGGCTGGA |
| | | AGACGAGCTTAAGGCCTACAACAAAGGCAAGCTCAAAGTTTTCGATCTTGTGTTTAGCTTAGAAAAACGTATCA |
| | | TAGAGGTTGCGAAACAGGACATTATAGCATTGTTGGATAAAAGTGGTTCTAACGATTCCAAGTTTAATGTGGAT |
| | | CACAAACCGTATATCAAATGGCTGGAAAATATGGAACTGATTAACAGCAAAGAAGCCGGCTTTATGAAAAAGA |
| | | TTCGTAACTCATTTTCGCATAATCAGTTCCCTGAATGCCAGACTGTCGAGCTGATGATTTCGCTGAAATCAGACA |
| | | TCCCGATAGCTACACAGATTGTGAATGAGTATGAGCGTTTAATATTACGTATTAGCAGTCAAATAGAAAAGATG |
| | | AAATAA |
| 330 | JW13155 | ATGGAGAAAGAATTGATTGTGAATGAGAACCGTGAACGTACATTAAAAGATGATCCTCAATATTTCGGGGGCTA |
| | | TCTTAATATGGCACGTCTCAACATCTTTAATATCTCAAACCACATAGCTAAGAGATTCGGTTTCACGATCTTACC |
| | | AAACGAGGAGAGAATCAGCTCTTCGTTTCTGTGTAATAAGGCACTGAAAAACGCGGATTGGAATCATATTCATA |
| | | GTAGAACAAAGCGCTTTCTTCCAATCATAAAAGTTTTTGATGCGGAGAGCTTGCCAAAGACTGAATATGATTTTC |
| | | TGATGTGGGAAGGAAAGAACTTCGCCTTAATGACCGATACCTTAAAATCAGTATTTTCGGAGATTCAGGAATTT |
| | | CGCAATGATTACTCTCACTTCTACTCTACCGAAAAAGGGACGATAAGAAAAACCAAAATCTCTCCTCAATTGGC |
| | | TTCATTTTTAGAAGTGAATTTCAAACGGGCCATTGAGTATTCTAAAATCCGCCTCAAAGACGTTTTACGAGAGGA |
| | | AGATTTTAACCTTGTAGAACGATTAAAGGTGTGCAATAACGGTAGCGAAATAACTACCGAGGGACTGGTTTTCC |
| | | TGATATGCATGTTTCTCGAGCGTGAACACGCTTTTCAATTCATAGGAAAAGTGAAAGGTTTAAAAGGCACGCAA |
| | | TATCCTTCCTTTATAGCAACTCGTGAAACGCTGATGAGCTTTTGTGTCAAACTTCCCCATGATAAGTTTGTCTCCG |
| | | AAGATCCTAAGCAGTCCCTGATTTTAGACATAGTGAACGAGTTAAACAGATGTCCAAAAACCTTATATGAAATT |
| | | ATTAGCGAAGAAGATCGGAAACAGTTCCGTCCGGAGCTCGATATAAAGAGTGTAGACAATCTCTTAAAAAACTC |
| | | TGGTAAGGAAGAGCTGCTGGATACGATGGAGTTTGAAGAATATATGGAATCATTGACCAAACGAGTCCGTCATT |
| | | CAAACCGCTTTCCGTATTTTGCATTACGCTTCATTGAAGAGATGAAACTCCTGAACAATATGCAGCTCCATATCC |
| | | ATCTTGGAAAGTTTGAACTGCAGAAGTATGACAAACAGTTGAACAATGAACCTTTTGAACGTGCTATTATTGAG |
| | | GATGCCAAAGCATTCGGTTATTTGAAAGACTTCAATGATGAAAATGACATTAAGGGCGAAATTGTAAGCGGAAA |
| | | AGATTCTGAGGGTTTCGCACAATTTTCTCCGAGCTACTTTCTGAAATACAACAAGATCGGGATTCGTTATAAGCG |
| | | TGACATACCCCATCTGATAGTTGACAAAAATAACAGCAAGGGTGCATACAATTTGAAACAACCTCTGGCGAAGG |
| | | CCTTTCTTAGTGCCTATGAGCTGCCAAAAATTATTCTCATGGAGTATCTTCAAAAAGGCCGCGCTGGCGAACTTA |
| | | TTGATGATTTTATCCAGCTTAACGACAATAAAATTATGACGTGGGATTTCATAGAGAATATCAAAAGTCAGTTGC |
| | | CGGATTGGAAAACTTTCAATAAGAAGTCGTTTGGACTGAAAGACAAAAAAGGGTATCAGACAGAAGAACTGAA |
| | | AGAGCTGAACACCCGTAAAAATCAGCTGAACAAACTCTTAGCGCCAGAGGGATTAAACCATAATCGTATTCCCG |
| | | AACGGATTACAGATTATTGGTTGAACATAGGCGACGTGAAAAAAAAGCGCTCGTTTTCTGATCACATTAAACTT |
| | | ATGAAACGGGAAGGTATGGAGCGCCTGAAACAGCTGGAAAAGTTCAAAAAAGAAGGTAAGGGTAAAATACCA |
| | | AAAGTAGGCGAAATGGCCACGTTCCTGGCAAGCGATATTGTCAATATGGTGATTAATGAGGAACGGAAAAACA |
| | | AAATAACGAGTTTTTACTACGATAAAATGCAACAGTGCCTCGCGTTATACGCCGATTCAGAAAAAAAGGCCTTG |
| | | TTCATTCAGATCATAAAGGAACTGAAGTTAAACGAAGAAGGCGGCCACCCGTTCCTGAAGAAGATTGACCTGCA |
| | | GAAAATCAGTAAAACGTCCCAGTTGTATGAACTGTACCTTACGGAAAAAGTTAATAAACCTATTCGTGAGGAAG |
| | | TTTTTAAGTATGATCGTAAAAAGGGCTATAAGGTCACCAAAATCATCTTACGAGATAAAAGTTGGATGGGGGAA |
| | | ATGTTTTACACTACTGAAAAATCCGAAAAGACTGGCAAAGATATTACCATCGTCCGTATGCCGGAAAATAAAAC |
| | | AGAAATTCCGTTTACAATCCGCCGTTGGGAAGAGAAACAGAAAACAGATCTGAAAGTTTGGTTTCATAATGTGA |
| | | CCAAGGGTAAAGACAAAACTAAAGACGGTCCCAAAGCGATTTCGTTACCGACGGATATTTTTGATGACGAGATT |
| | | TGCACCCTGATTAAGATTCAGTTGGACAAAATCAATGTCAAGTACAAAGAGAATCTTAAGTATAATGAGTTGCT |
| | | GAAATTGTGGTGGTGGAAATCTCGGGAAGACGACGTTCAAAATTTCTATCATAGCAAACGCCTGTACAAGGTTT |
| | | ACGATGAAGAGATTAATTTCACCCCGGATACAAAAAGTACATTTGTGGAATATTACGAAAAAGCACTGAACTCC |
| | | GCTTTCGAAAAACTGTCAAAAGCGCGTAAAAAAGAACGCAAAACGCACCGTGCCTTGCCCGATATCAATAAAA |
| | | AGGAAGTAGAGAAAGTGTTTAAACGGGCGATAGGGGAGACAGAGAAAGATATCCGCATCCTGCAGGAAGAAG |
| | | ATAGAATTTCCATTTTAATGCTGGAGATGTTGATGGATAATGCCACCCTCAAACTGAAAAATATCAGCTACTTGC |
| | | TTAACGACACTACCAGAATTCAGCAATCGATTACCGCGCATCTGTCATTTGATGAAAATGGCGAAAGTATTAAA |
| | | GAAGGGACTAAACCGACGATTACCAAAACAATTATCGCCGATCGGAAACAAAAAAACTATACTGTTCTGCGGA |
| | | AGTTTATATTTGACCGAAGAATGCCTGAGCTGTTTGAATATTATGCTTCAAATGAAGTAGCTTTAGATTGGGTTA |
| | | CAGACGAACTGCGCGCTTATAATACCGCGAAACAGTATGTATTTGACAATGTTTTCAATCTTGAGCAAAAACTT |
| | | ATCGAGAAAGATGAAACAGGGATCAAAGCAATGTATACTAATAAGGAAGGTACCCCGATAACCGGAAATATTC |
| | | AGTATAAACCGTACGTGCGCTGGCTCGAAAATAAAGGTCTGATCAACAAAAACCAAAAACGCTTTCTGACAATG |
| | | ACGCGAAATTGTTTTGCGCACAACCAGTTTCCGCAGAAAAAGACTGTTTTACGTATTGTGCCAAACCTTACAGAT |
| | | AATAACATCGCCCTGCAGATTGCAGATGCATATAAGAATATCACAGACGAAATCCTTACTAAACTGTAA |
| 331 | JW13156 | ATGGATGCAGAAGGAAAAAATGAACGCACTCTCCAGGACGATCCACAATATTTCGGCGGTTATCTGAATATGGC |
| | | GCGTCTTAACATTTTTAATATCAGCAATCATGTCGCCGATAAACTTAGCATCGCATTGCTGAAGAATGAAGAGG |
| | | ATATCAACAACTCCTTCCTGTGTAACTCTAATCAGAAACTCAACTGGTCAAAAGCTTATGCGATGACTATGCGCT |
| | | TTATGCCGCTGCTCAAAGTTTTTGATACCGAACGCTTACCTGAACATCGTACCAGCCAAAAAGATAAAGAAAAA |
| | | GGTAAAGAGTTTGGAATTATGTGTGATTCACTCCGCATAGTGTTTCGTGAGCTGAACCAGTTTCGGAACGATTAT |
| | | TCTCACTATACAAGCACTGAAGACAATTTTACACGCAAAATTGAGATATCCCCGGAATTAGCCGAGTTTCTGAC |
| | | ATGTAACTTTAAGCGTGCAATTGAATACACAAAACAGCGATTTGCGGACACCTATCAGAATGAAGATTTTATTTT |
| | | AGCTGAGCGGATGCAAATGGTGAAAAGCAATATTATTACGACTGAGGGGATTGTGTTTCTGACATCACTCTTTCT |
| | | GGAGCGGGAACATGCGTTTCTGTTCATCGGTAAAGTAAAAGGTTTAAAAGGGACACAGTATAAACAGTTCAAA |
| | | GCTACTCGAGAGGTGTTTATGTCTTATTGTGTCAACTTGCCGCAAGATAAACTTATATCAGATGATTACACTCAA |
| | | TCACTTACCATGGACATCATTAATGAATTGACGAAGTGCCCAGAGACTTTATACAATGTGTTGGATGAATCCGCC |
| | | CGTAAACAATTCATACCGCAGCTGGGCGAAGTCGAAACGGATAATGTGCGCCAGAATTCCATTAATGAAGCTGA |
| | | ACTCACGGATGAGAATGATTATGCACAGTATATGGAAGACATTACCAAAAGAGTACGGCACTCGAATAGATTTC |
| | | CGTATTTCGCATTACGTTTTATTGATGAACTCGATTTGCTGGGCAAGTTCAAGTTTCAGTTGGACCTTGGCAAAC |
| | | TGAAACTGAACACGTATCCCAAACCATATATGGGGGAAGATGTAGATCGTGCTATCACAGAAAACGTTTCTGCG |
| | | TTTGGGAAACTGAGCGATCACCAGGTTAAAGAAGAAGTACAGGCGAAGGTTGTGAAAGATTATTCTTCTGTACA |
| | | ATTTGATCAATTCGCTCCGCATTATAATATGGAATGCAACAAAATTGGAATTAGTCGTAAAAGTGAGACAGCGA |
| | | TTGTTATTAAACCCTCTGAAACCGGTGTGAAAAGTGCCTATAAACTGAAACAGCCGATTCCTGAAGCGTTCTTGA |
| | | GTATCAAAGAATTAAGTAAGATAGTGCTGTTAGAATACCTGAGTCCCGGTGAATCACAACGCTTCATTACCGAC |
| | | TTTATTTGTACGAATAGCGATAAGCTGATGAATCTGGATTTCATCAAAGAAGTAAAAGGCAAACTGCCTGTCTTC |
| | | GACAAACCTTTTTACAAAAAGGTTACGATCAAACGTAAAAGTTACACAAAACACGAACTGACTGATCTTGAGGC |
| | | CCGTAAAAACCTTTTGGATCAGGTTTTAGTTACCTATGGGTTAAACACGAAACAGATTCCAACCCGCATTACAG |
| | | AGTATTGGTTAAACATTCGTGATGTGCGCAATCAGTATATTATTTCAGAACGTATAAAGAGTATGCGAAAGGAT |
| | | TGCACGACCAGATTGAAAAGCCTGAAAAAATTGGATGAGACCGGTAAGGGTAAAGTTCCAAAAATCGGCGAAA |
| | | TGGCATCATTTTTAGCAAGAGACATTGTTTCACTGGTTATTGACCCGGTCAAAAAAAAGAAGATCACCTCTTTTT |
| | | ATTACGACAAACTTCAGGCCTCGTTAGCCTTGTTTGCTGATCCCGACAAGAAAAAGGAGTTCATGCACCTCGTTA |
| | | AAGATGAATTAAACCTGCTTTCGGTCGGGGGACATCCCTTTCTTATCAATATCGACTTTACCACGATACACTACA |
| | | CAAGAGATCTTTATAAGAAATATTTGGAAGAGAAAGGTTGCAAAATCACGAATGATCGGAAAGATATAAGTTG |
| | | GATATTCAACACTTTCTACAAGAAAGAACCGGGTAAAAATTCCTCACTGCCTGTTACCAAAGTTAAACTTCCGCT |
| | | GGATAAAAATGGTAACCCTGACTCCAAACGGCTGCCGCATTCTCTTTATTCGCTGGGCAACCGTAAAACAGATA |
| | | ACTCCATTGAAGAAATAGAAAAGTGGTTGTCCAATGTTAAACATGGAAAGATGAAAACTGATTGTACCAAACCA |
| | | GTAGACCTGCCTACGAATCTTTTCGATTCTAGATTAATTGAGTTACTGCAGAAAGAGACCGGCGACACCAGCCT |
| | | GGCACCAAAACTGAATGGATTATTTGTTACGTGGTGGAAGAAACGTGGGGACTCAACACAATCGTTTTACAACG |
| | | ATAAACGCAACTACGTAATCTATGACCAAGAGGTGTGCTTTCTTCCAAATACCAAGAAACTGTACCAAGATTAT |
| | | TATCAGAGCGCCCTTGATCGCGCTTTTCGTTTAAAAAGAGAAGAACGTCGGCAGGAGAGATTACTCAATATTCG |
| | | CTTACCGGATATACAGCTTAAGGATATGAAAAAGATCTTCAATATAACGCTGAGCGAAACTGAAAAGGAAATC |
| | | AGAATTTTACAGGAAGAGGATCGTCTGCTGTTGCTCATCCTGGAGAAGTTAATGGAGAAGGAAATAGCAGGCAC |
| | | ACTCAACTTGAAGAATGTCAATGATCTGTTAAACGAACAGGTGGATATCTGCCAGCAGGTCAAAACTTATCTTA |
| | | GTTTCAAAGAAAACGGTGAACTGGCATCGAAGGACGAACGCATGCAAATAACTAAAAATATAATATCCTACGG |
| | | TTCTCGTAAAAACTACGGCCTGCTGCGACGATATGTCCATGACCGTCGCATGCCTGAGTTATTTGAATATTATCC |
| | | TACCGACGATATCCCGTTTGAGGCGATTAAACAGGAATTAGAGGCCTACAATAAGGCCAAACAAATCGTATTTG |
| | | ATAAAGTCTTTGAACTGGAACGGGCGCTGACTGAGCGATTCAAAGACGAAGTTATGGCACATTTTGTGAACCGG |
| | | GACGGCGGAAGCCGCTTGAAAGGTAATATTCAGCATAGTCCGTATCTGAATTTGCTCGAGTCGAAAGGCATTAT |
| | | CAGTAAAGACGCACATCGTTTTCTGAAGGCTGTACGCAATGCGTTCTCACATAATCAATACCCCCACAAGAAAA |
| | | CAATGGAGATTTTTGTGAAGGAATGGAATACAAGCAACTTCGCCAATCAGATTGTGGACATCTACAAGCAAAAG |
| | | ATCGACGAGGTGATTGACAATATTAAGAAATAA |
| 332 | JW13157 | ATGGAAGAAGCTAATCGTTATATCTATGGTGCATACTTTAATATGGCGCGTGATAATTTCTTAAACACCATCAAA |
| | | CTTTTAGCTGATAAAATGAAACTCGGGGCCACATCTGGGTTCGGCAAAGACGGGAATGAGGTTAATGATATCAA |
| | | CGAACTGTTCGGTGATAAAAATACTTATGCGAACATTGAAAACGTCGTCGAATTTTATTTTCCGTGGATCAAAGC |
| | | ACTCGAGGGACGATTCAGTCTGGATAAAGGTGATAGAAATCTCAACGATATGAAAATGTTCTACAAATCCGTTC |
| | | TGACGGCATTTTTTACCGCGGTTGACTCATTGCGTAATAAGTACACACATTATAGTCACAAAGACCTTAACATCA |
| | | GAGAGATTAAGATAGAGTGTACGTTAGGCGGTAAGGATTATTGTATTGGACTGCTCAACGCTCTGGACTGCATC |
| | | TACGATAGTGCCGTCAATCTTTTAAAACTGCGCTTTATGGCAGATGAAGATGAAGTCGCCCATCTTCGTCGCTGC |
| | | AAAGCGGTTAATAAAAAAGTGGTAGTTCGGACAGAGAAAGATGGTTTTTATTATCGTCTGTCTGATAATGGCGA |
| | | AGTCACAGAAAAAGGTGTCATTTTTATTGCATCCATGTTTCTGAACCGAAAGTACGGGTTCTTGTTTCTCAAGCA |
| | | ACTGGAAGGCTTCAAAAGATCAGACGAAAAGAGATATCGGTTAACCTTAGAAGCATTTTTGGCCTTTAGCAATA |
| | | TTAAACCAGTTGATCGCCTGAAATCAGACAAACTGGACCGGGCGAGCCTGGGTTTAGATATGTTAAACGAACTG |
| | | ACCAAAATTCCCAAAGAACTGTCCGAGACATTATCAGTAGACTGCTTATACAAGTATCTTGCTTCAGATGGGGA |
| | | AGATGATCTGCGTAGCCGTATTCGCTATCAAGATAGATTTGTTCCCTTAGCATTGGAGTTCATATCGCAATCCGA |
| | | CGAATTCAAGGATTTCCGTTTCTACACGTACGTTGGCAATTATGTTTATAAAGGCTACATTAAGCGTCTGATCGA |
| | | TGGTACTGATAAAGAGCGTTACCTTTCAGATCGACTGTGTGGTTTTTACAAATCGGTGAATGATGCAAGCTCTGA |
| | | CGCAATTGCCCAGAAATATGGCGTGGAAATTAAAGATAGTAACGAACCGGACTACATGTTACCGGATAGCTTTC |
| | | GGCCTCATGTATTACGTGCTACGCCGCATTTCGTCATTAACAACAACAATATTGGCATCAAGATATGCGGCAAC |
| | | GACTGCCTGCCAGTCGTCAATGGAAAAGGTGTAGAATCTCCTGAACCTGATTATTGGCTGTCGATCTATGAGTTA |
| | | CCAGCCATGTTATTTTACGCTTATCTGCGGGAGAAAAACGGTAAACTGCTCAAAGACTATAAGTCAATTCGTGA |
| | | GTTAATCGAGGATGTGGAAAAAAAAGCGGACGAAAAAAATGATCGTGATAAAGGTGCACTGATGGCGCGTCAT |
| | | ATTGATAAAGAGATCATTTGGACTCAGACTAAACTTGATGAAGTTAAACGCTTAGAAGAAAAAAAAGTGGCGG |
| | | CCTATGGAAAAAAAGGTCGCGTTGTGTTAAAAAGTGGGCGCATGGCCGATCTGTTGGCCCATGATATGGTTCGG |
| | | TTGCAGCCTGCCACCAAAGGAAGTGATAAAATAACTGGCGCTAATTTTCAAGCACTCCAGGTGTCCCTGGCGTA |
| | | TTTTAAACGAGATATACTGGCCGATGTTTTTTCTCGCGCTATGTTGACCACAGGCAATCATCGCCATCCCTTTCTT |
| | | TATCGTATCGATGTGTCGCACTGTAGCTCTCTTAGAGATTTTTATGTGGCGTATCTGGGTGAGAGACGTAAGTAT |
| | | TTTGAGGACGTCGCCAAGAAAATTACCAAAAACAAACTGAACACCCCGTGCCATATCTTGCGGCGCTTGCAGCG |
| | | AGAAGGTTCAGGCGAGGAAGCCGGGAAAGACGTTAAACCGAAGTTTCTGCCACGCGGCATCTTTACGGGATCC |
| | | ATAAAGAGTTGTCTTGAGAAATCGGCTCTGAACATTAATATACGTAATGCACGGAATGATGTTAAACCTGCGAT |
| | | TAATGCGGCTTATCTCATATTCATGTATTACAAAGAGATCGAAAAGGGTGAGTTTCAGGGTTTCTATGGCGAGA |
| | | AAAGACGCTATGATATACTGGAAGAAGGGAAACCGTTGGATCTTGATGAACGGAAAAAAGCACTGGCATCTAT |
| | | CAAACCCGCTAAAATCGATGTGAGCGAGGCTAATATGCCGATGAGCAAAGAAGAACACCTGATGCGCAAACGT |
| | | TACCACGCTGTATGCAATAATGAAAGCGCAATCCGCATGTATCAGGTACAGGATATACTGTTGTTACTCATGGC |
| | | GAAGGATATCTTCAAGAAGGCGCTGTCAGAGGTGGTGATGTCTAAGAAAATTGGACTGGAAAATCTTAATGGCA |
| | | TTTTCGACGCGCCAGTTAACTTCGTTAAAAACTTTGACAACATTAAACTGACTGCAACTGGCATTAAAATTAAAG |
| | | ATTATGGAAAGGTATGCCGTCTCGGAACAGACTTTAAGTTCAATAGCCTGATAAAGGCCTTCCATAAAGTATAC |
| | | AGTAAGAGTGTCGAAATGGACTATTCTGACTATCTCAAAGAAGAAGAAGAATTTGAAAAATACCGTTTGAACAT |
| | | GGTGAAACTTTGTCGCGAGGTGGAACGGGGTATTACGGAAGATTTGCACCTGTCTCTGGATGGAAAGAGTCATC |
| | | TTTCATTCAATGACGACGTGATTAAGTCGTATAATGACAAGTACAATGTGTTTAACGGGGACGACCTTACGTTTT |
| | | TTATTAATGCCCGGAATATGTTTATGCACGGCGACTATAAGTATGAGTGTGTTAAGTATGTAGTATCCGAGCATT |
| | | TTAAGGGATCCTTAAACGATGTAAGCTTTGCCAAAGAAACATACGGTCACTTTTGTAATCTTTTGGAAAGCATGC |
| | | GCAAAAAAACCGGGTTGCGAATAGATATTTAA |
| 333 | JW13158 | ATGACTGAGACCAAAAGTAAACGTGAAGATATTGCAAAACCGCCTGCCGCAAAAAGTCGCTCCAAAGCTGCTG |
| | | GCTTAAAATCCACTTTCGCTGTCAATGGATCCGTGCTCTTGACTTCTTTCGGGCGAGGAAATGATGCGGTACCAG |
| | | AAAAATTAATTACCGAAAAAGCAGTTAGCGAGTTGAACACAGTCAAACCACGCTTTTCAGTGGAAAAACCTGCA |
| | | ACCAGCTATTCAAGTAGTTTTGGCATAAAAAGTCATATATCCGCAACCGCGGATAATCCCTTAGCCGGTCGGGC |
| | | GCCAGTGGGCGAAGATGCGATACGTGCAAAAGAAGTGCTGGAGCAAAGAGTATTTGGCAAAACTTTTCCTGAT |
| | | GATAATATCCACATTCAGCTGATCTACAACATCCTCGATATCCGGAAAATCCTGTCCACTTACGCCAATAATGTT |
| | | GTGTTCACGATAAACAGCATGCGTCGTTTAGATGAATATGATAGAGAACAGGATTATCTTGGCTATCTTTATACG |
| | | GGTAACTCGTATGAACGCCTTCTGGATATTGCTGACAAATATGCAGTAGACGGCGAGGATTGGCGTAACACGGC |
| | | GGCCGGGATAAGTAACGACTTTGAAAAAAAACAGTTCCAGACGATTAATGGTTTCTGGGATCTTCTGGACATGA |
| | | TAGAACCATACATCTGTTATTTTTCAGAGGCTTTCTTCTGTGAAACAACAGTTAAGAATCCAGATTCTGGTCGCA |
| | | TCGTGCCGTGCCTGGAACAGCGAAGCGATGGTGATATCTACAATATCCTGCGCATTCTCTCAATTGTGCGCCAAA |
| | | CTTGCATGCATGATAATGCTAGCATGCGGACCGTCATGTTTACCTTAGGTCAAAATTCTGTACGGGACAGAAAA |
| | | AATGGATTTGACGAATTAGCAGAATTACTGGACTACCTGTATGATGAGAAGATCGACATCGTTAATCGCGACTT |
| | | TCTCCGCAATCAAAAGAATAATATTGAATTGCTCAGCCGTATCTATGGCTCTTCGGCGGATAGTCCGGAACGGG |
| | | ACCGACTGGTACAGAACTTCTACGACTTTCGAGTGCTGTCGCAGGATAAGAATCTGGGTTTTTCTATTAAAAAGT |
| | | TACGTGAGAAGTTGTTGGATAGCCCTGCATTATCGGTAGTACGTTCTAAAAAATACGACACAATGAGATCAAAG |
| | | ATCTATTCCTTAATTGACTTTATGATCTACAGAAAGTTCTCGGAAAATCACGTCGCGGTCGATGATTTCGTTGAA |
| | | GAATTGCGTAGTCTGCTGACAGAAGATGAGAAAGAATCTGCCTATTCTAGATGGGCGGAAACCCTGATTAATGA |
| | | TGGGTTTGCGCAGGAGATTTTGGTCAAACTGCTGCCACAAACAGACCCGGCAGTAATTGGAAAAATTAAGGGAA |
| | | AAAAACTGCTGAACGACTCAATCGCAGGAATTAAGCTCAAAAAGGACGCGAGTTTCTTTACGAAAATTATTAAT |
| | | GTACTCTGTATGTTCCAGGATGGGAAAGAAATCAACGAGTTAGTGAGCAGTCTCGTGAACAAGTTTGCCAATAT |
| | | CCAATCATTTGTGGACGTTATGCGCTCGCAGGGCATTGATTGTGGGTTTACCGCTGACTATGCCATGTTTGCAGA |
| | | AAGCGGCCGCATATCCCGCGAATTACATATACTTAAAGGCATAGCCCGCATGCAGCATTCAATAGCTGGCTTAG |
| | | GGGATGTTAAAATATATTCCTCTGATGATAAGTTTCATGGTGTGTCGCGTCGAGTCTATACCGATGCCGCATACA |
| | | TACTGGGCTTTGGTGAACGTTCAGAAGATAATGATGGTTATGTAGACGATTATGTTAGCAGCAAACTGCTGGGC |
| | | GGTGCGGATAAAAATTTGCGGAATTTTATCACGAACAACGTTATTAAAAACCGACGTTTCCTTTACACGGTGCG |
| | | GTATATGAATCCCAAACGGGCAAAAAAACTGGTCCAAAACGACGCCCTGGTTGTCCTCGCGTTGAGCGGCATTC |
| | | CGGAGACACAAATAGATCGCTATTATAAGTCCTGTATTGAGAAACGCAGCTTTAATCCGGATCTTAACGAGAAA |
| | | ATTGCGGCACTGAGTGAGATGATTACGACTTTAAAGATTGACGATTTTGAGGATGTGAAACAGAACCCGGAGAA |
| | | GAACGCAAATTATGAGGCGAAAAAAAATCAGAGAATTTCTAAGGAACGTTACAAAGCTTGCATCGGTTTGTACC |
| | | TGACCGTTCTTTATCTTATCTGCAAAAACCTTGTTAAGATCAACGCTCGGTATAGCATAGCTATTGGGTGCTTAG |
| | | AGCGTGATACTCAGTTGCATGGTGTTGACTTTAAAGGGGCGGCGTACATGACACGTGATGTCTTCATTGCCAAA |
| | | GGATGGATCAATCCGAAGAAACCCACAGTTAAGTCAATCAAAGAACAGTATGCCTTCTTAACACCGTATATTTT |
| | | TACCACGTATAGAAACATGATTGCCCACCTTGCCGCCGTGACCAATGCTTATAAGTATATTCCCCAAATGGACCG |
| | | GTTTAAAAGTTGGTTTCACCTGTATCACACTGTTATTCAGCATTCTCTGATTCAGCAGTATGAATACGACCGTGA |
| | | TTATGGACGTAAAGGGGCTCCGGTTGTGTCAGAACGCGTTTTACAATTGCTGGAACAGTGTCGAGAACATTCGA |
| | | ATTACTCTCGCGATCTGCTGCATATACTGAACTTACCTTTTGGTTATAACCTTCCTCGTTACCTGAACCTTTCATC |
| | | CGAGAAATACTTCGATGCCAATGCAATTTAA |
| 334 | JW13159 | ATGAACATTCCTACGATCATAGAGCAGAAAAAACAATTTTTTGGCACCTACTCTGTTATGGCGCTGCTGAACGTC |
| | | CAGACAGTGCTGGATCACATTCAGAAACTTGCAGACATAGAGGACATACCGTGCTATGACGAGAATGGGAAAG |
| | | AGAAAGATAAGCGCGAGAACCTTTGGGAGCATAGTGTAATGCTGTACATCAATTATAAGAACAATTCCATCCAG |
| | | AAGGCTCCCGAAAAGACCGCGTTAATCATTGATAAACTGATGGCATATTTTCCGTTTCTCAAAATCATGGCGGA |
| | | GAATCAGCGTACACATAAAAACAAGAAATATCAGCAGAATCGCCTCGAGGTGAACAGCGATGATGTGTTCGAT |
| | | GTACTCAACTGCTTATTTCGGGTGGTGAAAAAATATCGTGATACCACCACGCATTACATGCTGCACGACGAATG |
| | | TTGGGATGATAATAGCAAGTTTCTCCGTTGTTCGGAACAGATTTTAGCGACGATGTTGAACAAATACTACGACGT |
| | | TGCCTTACGTAATATGAAAGAACGCTATGGGTATACAACTGAAGAGCTGGCCTTCATCCAGAATCATCGCTATA |
| | | AGATAGAATGGGAACCAGATTCACGGAAACGTCCACAGTCCAATACGCGGTTTTTCTTTTCCCTCGTTGACAATA |
| | | ACGGCGATGCAAATCCCGACACGCTGCATCTTTCAGGTGTGGGAGTTGCCGTCCTCATTTGTCTTCTGCTGGACA |
| | | AACAGTACATTAATCTGTTCGCACAGCAGTTACCGGCGATCCAGAAATTTGAACCTCAGTCGGCCGCCCGGAAA |
| | | ATGCTGGTTCGGAGCATGGGTATTCACTCCATTGTCCTGCCAAAGGACCGTATTCATTCACAAAAAGATAATATG |
| | | TCGATTGCCATGGATATGATAAACGAAATGAAAAGATGCCCGAAAGAACTCTTTGACACGTTGTCTCCATCAGA |
| | | TCAGTCCCGGTTTCGAGTAACATCAAGCGATCATAATGAGGTACTGTTCCTCAGACACAATGATCGCTTTGCTCA |
| | | GTTGGCATTGCAATACATAGACTATTGCAAGTGGTTTAACGGGATTCGTTTCCATGTCAATATGGGTAAGCTGCG |
| | | TTATCTGTTTAATGCTGAAAAGAATTGCATAGACGGGATTAATAGAGTCAGAGTTATAGAACATGCCTTAAACG |
| | | GCTTCGGACGTATTGATGAGATGGAAGACCTGCGAAAAAGTGAAGTAGGTACTTTTGCAAATACTGGTGTTGCA |
| | | ATACGAGATTTTGAAAATGTTAAACGCGACGATGCTGATCCTAATAACTATCCGTACATCGTAGATACCTATAC |
| | | GCATTATATCCTGGAGAACAACAAGATAGAGATGCTTTTAACCGATACGAACAGTCTCCCCACAATTACCGAGA |
| | | AAGGCGGTAAGTGGTATGTGGGAAAAAAGGCGCCGACATGTCGAATGAGCATTTTTGAATTACCGGCCATGATG |
| | | TTTCACGCAATCTTATTGGGTAACCAAGCAACAGAAAAACGCATTAAAACTGTTTGTGCCAATTACTCGCGCTTA |
| | | TTCGAGGCATTGAAACAGGGTGCGGTTAATAAAGACAATATCGCCAGCTTTGGTATCGCGGAAGAAGATATTCC |
| | | GCAAAAAGTTTTAGATATCGTGAACGGTTGCGCTCAAGGAAAGGACGTCAATATCTTCATTAAAAAAAATATCG |
| | | ACGAAATGCTGCACGATACAGAACGTCGCCTGGAACGTTTACGTGAAGATCGCGAAGCTGTTAACGCAAATGAT |
| | | AACCGTATGGGTAAAAGAGGCTTTCGTCAAATACAACGTGGCAAACTTGCTTCTTTTCTGGCAGAAGATATCGT |
| | | CAAACTCCAGCCGACCCTGTGTAATGGAACAGATAAACTTACTGGCTTAAACTATCGTGTTCTGCAAGCATCTCT |
| | | GGCCGTTTATGATTCTGGTGGTATGAATGAGGCACGCCAGCAATTCAAAGCGTTATTTGTCAAAGCGCATCTGCT |
| | | GGGGACTGATCCTCACACTTGTCATCCTTTCCTGCATAAAGTGTTTGCTCGCTCTATCCCAGATAATACCATTGAT |
| | | TTCTATGAACGGTATCTTGCAGAACGTGAGAGCTATCTTAGCACCATTCAGAAAAAAGTAGCCATGGGTAAAGC |
| | | GGTGAATGTGCCGTTTATCAATCGTGCTCAGTCAAAGTGGTCTGCTCCAACGCAAGAAGCTTTAGGAAATGCTT |
| | | ATACAGATGATATGGCCATTGAACTGCCAAGACAAATGTTCGATGACGAGATTAAAGCGCACTTGAAAACACTG |
| | | CCGGAAATGGCGGACATAGATTTTGATAACGCAAACATTACGTTTCTGATTGGCGAGTACCAGAAACGGGTATT |
| | | GCATGATAATGTCCAGCATTTTTACTCTTGGCAACGAAACTACAGATATATTGACATGTTGAAATGTGAAACCG |
| | | ATCGAAAGAACTGTTTATGTAGTACGTACACCTCAGTAGAAGAACGTGAAGCGTTATGGGGCCGTCGTAAAGAA |
| | | TGCATTGAAAAATATCGCCAATGGTTTACGCGTAAAACTAGCAATGATCGCGGGCGATCCTTAATTAGTGCTGT |
| | | GGATATTGAAGAGATATTAGATAAACGGCTGAGTAATGCGCGGAACGAATATCAGAAGTGCGAAAAGACTATT |
| | | CGTCGCTTCAAAGTGCAGGACACCTTGCTTTTCATCTTAGCCAAGAAGGCCCTGACTAATTTTGCTGATTATAAT |
| | | GGAGAGCGGTTCTGCTTGAACGATATAACCCCTGAAGGTGATAAAGGGATTCTTTCACAGTTAATGCCTATGAT |
| | | TTTTCGCTTTGAAAAGAAAGGCAAGGTATACACAATTAAGAGTGAGTCGATGAAACCCAAAAATTATGGGGACT |
| | | TTTTCGTGATCGCGAACGACAATAGAATATGTAACTTACTGGACCTGATCGAAGATAACATAGTGTCCAAAGAG |
| | | GACATTGATAAAGAGCTGCAAAAATATGATCAGTGCCGCCCCGAAATGGCCAAACTTGTCCTGGATCTTGAAAA |
| | | ATGGACTTACTCTAAAAGTCCGGGCTTGATGTATAAAGAGAAAGTGCAGTTCAAAGATGTTTTGGACGAACTGT |
| | | GCAAGCACACCAATCTTACAGAGCGCCAGAAGTTCGTTTTAGGAAAAGTCAGAAATGCCTTTGAACATAATGAC |
| | | TATCCTGAGAAAGGCATTTTAGAAATCACTACCCTTCCACAAATCGCAGCTCGTATGAAGGACCTGTTTGATGA |
| | | ATATGCAGAGGTTGAATAA |
| 335 | JW13160 | ATGAAAGAGAAGATTAAAAACAAAAGTAGCATTATCCGTATTATTATGTCGAATTACGATGACAAAGGCTTAAA |
| | | AGAATTCAAGGTGCTGTATAACAAGCAGGGTGGTGTTGACACATTCACATGTAAAACTGATATTGTGGATGGAA |
| | | CAATTATTTTCTTAGAGATAGAAAAACACCTTCGTGACTTTGGCGATGATTTTTCCTGGGATATTTCATCTGATG |
| | | GAAAAAGTGTCGAAATAACCAAGCTGATAAACGGCAAAGAAACGCGTAAGTATAAAGTTTCTATCAAAAATTC |
| | | TTCGACGAAAGATAAAAAGAACCTGGTCGAATTAGAAGTTGAAGATCTCAAAGAGTCTGCAATTGATCGTCGCC |
| | | GAACCAAGAGTTCTACTAAACGGGTATTGTTGAGCAAAGACGTCATGGAACGTTATGCTGAAATTGCCTTTAGT |
| | | AAGAAAGAACGCTGGGAAGAGATCGATTCCCAGAAAATCTATAAAGTTAAACGCTTCTTAGATTATCGTTCAAA |
| | | TATGCTGATCTACTTCCAATTCATTAACGATTTCTTGACCAAAGGCATACCAGACGAGCTGGACAAAAATGGTG |
| | | AAATTAAACAGCTGGAACTCTGGAAACTGATTGACGACGATGAAACGATCTCAGATAAAAATCTGAATCAAGT |
| | | GTCCAAAAACTTGTATACATACATTTCACAGGAAATTAAAGATTCTCAGACGCGGGCCGAAAATAATCGGGAGA |
| | | AAAATAAAGAGAAGGAGCACTTTAAAGAGTTTTACGCTTTTAATGATATTAGCGAAGAATCAATTCGTGAGGAC |
| | | GTTAAAAAATTCATCTATCTTTATGCAAATTTGCGCCATAACCTGATGCATTACAATTACTCGTTTTTTGAAAACC |
| | | TTTTTGAAGGTAAGGACCTGGTAATAGAGAAAACGAAAAGTTTACTGTCGAGTACCTTAGATCTTAATATCTTCA |
| | | AAGAGTTATCCAACATAGTGGAATTAAGAGAGGAAAATAAAACTAATTATCTTGATGACGAAACCACCATTAG |
| | | AGTGCTGGGCAAAGAGAAAAAAGCAAAAACACTTCACAAAATCTATAGTATACTGTGTTCACGCAAGAACGGA |
| | | TTCAATAAGTTTATAAACTCCTTTTTTTCTACTGATGGTATTGAGGAAGAATTCTTGAAATCGGAAATTAAAAAA |
| | | GACTTTCTGGAACGCTTAAACTGGGTAGAGAAATCCCTGATTGAAAAAATCAATAATCCGCCAAGCGATACGAA |
| | | ACTGAAATATAAGAATGACAAAACTATTGAGAACATGACGAAAGAAAAAGAGGAGAAACTGGAATTAATTAGT |
| | | CTTTTGAACCCACAAGTGTCCGATTACAAAACCGAAAATTTCACCCCGTATTATTGGGACATACATCAGTCCCCT |
| | | AGTTATAAGAAATTGTATAATGATAGAAAAGTACTGGTTTCGGAATTGAGCAAACTTATTGCAATTGGAATTAA |
| | | TTCAGACACTAAGAAACGTATTACAGATCTCAATGCCGAACTTCTGAAGATAAAGATCAAGATGGAAAAAATTA |
| | | CCAAACTGAATTCGAAAATACGATTGCAATATAAACTTCAGATGGCCTTTGGCTTTATCTATGCGAATTACTCAA |
| | | AAGTGTACAAAGAAAAGCGAGTTCTGAACATTAACGGATTTGTTCAGAATTTCGATCCAACCAAACTTAATAAA |
| | | GAAAAAGAATTGGAGAGTCGCTTAATCTATCTTAAGGCTCCGTATAACATCTTTGAAGACAATAAAAGCCTTGA |
| | | CTTTAACATGAAAATTGTCGAGAACATCCCAGTAAGCGAAAAAAGTATATTTCGGATCAAACCGGAAAATAATC |
| | | TCAGCAAGTTTTACATCCTGAGCTACCTGTTACTGCCTGTCGAGTTACGGGGTGATTTTCTGGGCTATGTTAAAC |
| | | ATCACTATTATGGGATTAAGAATGTCGATTTCGAAGAAATACCGGATATTAAAGAAGATAAACCCAATGAAAAT |
| | | TCGGATTCTTTCTTTCATAACCTTCGTCTTTTCGAGAAAAACAGCAAGAAATTTGAACTGATAAAATATAGACTC |
| | | GTCGAATTTGGGAATCTTAAAGATCATCTGCCACGTATCTATGAGAAATTTGGGATAAAACCGGATGTGTTAGA |
| | | GTACATTGAAAATAGCGGGAATAAGGACTCAAAACTCTTTGATCGGAACATACTGTTACCCATCATGAAGTACT |
| | | ATCAGCATATCTTCAAACTTCTGAACGACATCGAAGTTCATGCTTTATTACGCTTTTCTGAAAAAGACTCAATTA |
| | | GTTTAGATGAGTCTATTAAAGAGTGCAGTAAAGGTAAGTTTCTCAATTTTGGTAAGCTGTTATTTTTGAGCCGTT |
| | | ATGGCCTCGAGGCGAAAAAAGATAATAAGTTTAAAGATATTTTTAACAGAGAGAATGGGCTGTCGATTACGAA |
| | | AGATGATGCGAAGACGGAGCGCAAAAAATACTTTGAAATCTTCGAGACGCGGAATAAGATCGCACACCTGAAT |
| | | TACAAACAACTGTTTCACGACCTTCTGTTTGATTCTAATATTAATATCAACAAAGAGCTCGAAGGCATTATTCAG |
| | | GAAACTAAAACAATTGGGTTGAACGCCCAGACTCTGGGATATAACTTTCTGAACGATTTTTACATGAGAAAGGA |
| | | AATGTTCATATCAAACCAGAAAAAGAGCTCAATGACATTAATCAACAATCCCTTATCCAAGGATAAAGACACAA |
| | | AGGAAATTGGCCTCTTGAAGCTGTATGGTTTAAGCAAGAGTCAACCGAAAGATCTGATCCTGGCGAAATATAAA |
| | | GAATTGATGAACCTGATTGAGAAAACAGAGGACAGCATTTTGAAAAAGAAAGATTTCCTTCCTGTAAAGGAAGT |
| | | TTCAATTACCGTAAAGAAATCTACCCCCAATAAAAAGGGTATAATGGTGGAGTTACCTGAAATTTTACAAATTA |
| | | AGGACCTGAACGAAATGGATCTCCTGGCTTATGCGTCAAATATTCGCGGTAAGCTTCACAAAGACTCTTCCGAT |
| | | CTTTTCGGTATCTACAAAAAATTGACCATCAAAGAGCTGAAGAAAAAACTGATAAACCTTTTCATCAAAGGAGA |
| | | AAAGCGCTATCTGAATCTTGAATTAGTGAATAAAACCGGGTATATGGCCATCTATGAATCTACCGGCCTGTATCC |
| | | GAAGAGTTACGAAATTCTCAACCATGAAATCTCTTTTAGCGAGATTTCCATGAAGAATTGGTACGAACATGATTT |
| | | TAAACCTATATTTCAGATAGATGGCTCACTGCCTAATAACACTGACTATAAGAATGGTGTTTTCATCTACACATC |
| | | CCCGTATGAATTTCGAGACAAAGAACTCATGAAAAAACAACGTACAGTGCATAAACGTGATATAGAAAAGACT |
| | | TTTTACAATGAGAACGATATGGATTATACTGGGATCTATAATCAGAAAATAAAAGCACTGTATTAA |
| 336 | JW13161 | ATGTTCAACGATAGTCAGGGCAATCAGATTGAGCAAAACGACAAAAGTTTTCTGGAAATCTTCTTAAACCAGGC |
| | | ACGTCTTAACGCGCACATTACACTGTGTCACATTTCAGATCTTCTGGGGGAAAATACCGTTGATGAAGATTCGTT |
| | | AGCCATTATGCCTGTCTTGCGGTTTCTCGAATCGGATAAAGACGCGGTAAAAAGTGAACGCACTTTCGAGCTGG |
| | | TGGAAAAACACTTTCCAATGCTGAAAGTCATCTATGGTAGCGGACCGAAAAAAGAAGAGGTTACCTTCTTGGGC |
| | | CGTACCAAAAAGTATAAGGAAATACTGGGCTGCTTCTTAAAGGCAGTAAACGGAAAGCGGAACGAATATTGCC |
| | | ATGCTTATAGCAAAAGTTCCGGGGATTACGATATCAAAACACTGACTCGCCATCTTGAAAACTGCTTTGATGCCT |
| | | CTGTGAGAAAAATTAAAGAGATCCGCTCCTTGGAAGAGGAAGAGGTTTTCCATCTTCGTAGAAAAACAAGCGA |
| | | AGGTAAAGGTAAAGAGAAAAGAGTGGTAGATAATCCAAATTTCCACTATCATTTCAAGAACGAGGAAGGCCAA |
| | | CTGTCCGAAAAGGGATTAGCCTTTTTAGTGGCTATGTTTTTAGAAAAAAAAGACGCTTATTTGTTTCTCAAGCGT |
| | | CAGCATGGCTTTCATCGTGGAGAATCTCCGGGCGAAAAAGCCACCCTTGAATCTTTTTGTGTCTTCCGGATACGC |
| | | TTGCCAAAGCCTGTTATGACCTCTGATGTGGACAAGAATGGGCTGGCCCTGGACATGCTGAACGATCTTAAAAA |
| | | ATGCCCTAAAGAGTTGTTCGAATTACTGGCCGAAGAAAAACAGCAGGAGTTTCGCGTGACAGATTCTGAAGATG |
| | | GGGATGAGGACGGCAATGAGATACTGATGCGCCGTTATTCAGATCGATTCCCCTATTTTGCACTGCGTTATTGTG |
| | | ATGAAAACGAGGTGTTTAAGAACATTCGCTTCCACATAGACCTTGGGCGTTACTACTTTAAGTTCTACGAAAAA |
| | | GATACAATTGACGGAAATAAGTACCAGCGCGCTCTGGATAAACGCCTGAAAACATTTGGTCGTATTAAAGACGT |
| | | CAAAAATAAGGTTCGGCAGGAATGGGGTGATATAATCAAAGCGCCAGACGAAATTTCGGATGAGCAGAATGAA |
| | | CCGTATAAGACCAATAGCACTCCGCATTATAATTTGGTTGATAACCAGATCGGGTTCACTATCTCCGGTGACTGT |
| | | AAGCTGCCTGATGTTAAACAACCGGAAGGAAAGATCTTACTCCAGAAGCCGGATGCCTGGCTGTCTATCTATGA |
| | | ACTGCCAGGCATGCTGTTCCACGGCTTAGGCTATGGTTTTAAAAAAACTGAGAGCCTGATAGAACAGTACATCA |
| | | TTCGGCAGCGGAAAATCTGTGAGGAAATTTTTCGCACAGGAACGATTCCAGCGAATGCAGGCGAGTTTTTACCG |
| | | AATGCTATCAAGGAAATAGGCACGGGTAATGAGACATCAAACGAATATGCGAAAAATAAACTTGATCGTAAGC |
| | | TGTCAGATACGCGCCAACGTATTAGAGCGATTAAAGCAACCAAAAATAGAATTGCCGATAAAAGTAACAAACC |
| | | GGGTAAGCGCAAGTTTTTTGATATTCGTGCTGGCAAACTGGCTGACTTCCTGGCCCGTGATATTATGGCATTACA |
| | | GGAGTTTGATCCAGCCAAAGAAGGTAAGGATAAACTGACTTCTATTAATTTTCAGGTACTCCAAGCGACTTTGG |
| | | CATTTTATGGGGCCAAAAAAAACACGTTAGCTGGTATGTTTAAGAGAATAGGCTTACTGGAAGGAGATAACCCT |
| | | CATCCCTTTATGAAAGAAATTGAACCTGGCAAACATAATAGCATTGTAGGTTTCTATGAAGCGTATCTGGATCG |
| | | GAAAGAAGCGTATTTGGAAGAATGCCGCAAAGGAGAAAACTTGGATAACTACCAATTCCTTCGTCCGTCCCGTC |
| | | AGCGATATGCAAAGGGTAAAAGAGACCTTAAAACCATAACACGCCAACTTCTGGATAACCCCGTCAATATTCCG |
| | | AATGGTTTCTTTCGAGAAGAAATTCGTGAATTTGTTTGTGAACAGGACGCTGGTCTGAAAAGTCGGAAAATGAA |
| | | TACTGCATATATGGTAGGCGCTAATTTTGAAAAAACGTACGGGGGCCAGCAACCTTTTCAGTACTATCAACGTA |
| | | ATTATCCGGTCGTACTCAAAGCTCAAGAATACTGTAAAAAGAGAGAAAATCGTGGCATTGCAGAGGTGTTACGT |
| | | AGTATTGCAGGTGAAATGTCATACCGAGAGATGGAAGCCTTTATTAAAAAAGAAATACCCGATAATGGTCGGTT |
| | | TGAACCTGAGAATCTTAAGAGAAATCTCTTAAACGGATGCAAAGACTTTAAAAAAAACGAACGACTCCTGCGCC |
| | | GTTACAAAGTGCAGGATATGGTCGCCTTTGCTATGGTGGAAAAAACATTGAAGGAACAGCTGTCATTTGATGGT |
| | | AACGTTGTTAAACTCGAGGCAATTACGCCGACAGAGGAAAGCCTGTTCAAAAAACCGATCTTATGTTCAACTGA |
| | | TATCGCGATTACGTTTAACACCAAACCCACGCACTATGACAATGGATATGTTGGGTTTATCCAGGAAAAATATG |
| | | CCGGGCATTATGCATCTGAAGGCAACAAAATCATCTTGAAGTATCGCATAATATCGGAGAATACCAAACTTAAA |
| | | GACATCGGGAAGTACCGTCGCTACCTTTATGACCGTCGGCTGCCGGGTCTCTTAATTTGGAAGTATGCGCCTAAC |
| | | GCCCAGTCAGACATTGAGATCAAGTATTCCGAGATCGAGGAAGAGATCAAAGCTTATGAACAATATCGCCTGCG |
| | | AATTGCAGAGCAGCTGTACGTATTAGAGACCAAAGTGATCGACTGCTACATTTGCAAAGATGAGCCAGGAGAG |
| | | GACTACATAAGCTTTAATGGTATTATAGATACGATTAAAGCGGAACAAGCCGGTAGCGAGGAGAAATGCGATA |
| | | CCTTACTCAGTATACGGAATGCAGTTTATCATAATCAATTTCCCGCGTTTATGGATGCGATCGAAAAAACGGAA |
| | | GGTGACAATATTGCGGGGAAAATGCTGTCGATAACTGAGGACTACGTTAAGTGTACCGTCGAGCAGATGAATGA |
| | | AGGAGATAAAGAATTCGTTAATGTGTAA |
| 337 | JW13162 | ATGAACAGTGTCGAATTGAAGAAAGAAGAAGCCGCGTTTTATTTTAACCAGGCGGAGCTGAACCTGAAAGCGA |
| | | TCGAAGATGATATTTTTGACAAAGGGCGCCGGCGCACACTGCTGGACAATCCAAGAATCCTCGCTGAGGTAGAG |
| | | AACTTTATTTTTAACTTTAAAGATGTTACCAAGAATGCGAAAGGCGAGATCGATTGCCTGATTTCGAAACTCCGG |
| | | GAATTACGCAACTTTTATTCGCACTACGTTCATAACGACAATGTGAAAGTACTTTCGAAAGGTGAAAAGCCAAT |
| | | TTTGGAAAAGGATTATCAATTTGCAATAGAGCGGACCGCTTCAGAAGATGTTAAACTGGAAATAGTTGATAAAC |
| | | GCGACAAACTTACCGATGCCGGGGTGCTGTTTTTATTATGCATGTTCCTGAAAAAGTCTCAGGCAAATAAACTTA |
| | | TCAGCGGCATCTCAGGTTTTAAACGGAATGATCCGACCGGCCAGCCCAGAAGAAATCTTTTTACTTACTTCAGTG |
| | | TTAGAGAAGGTTATAAAGTAGTTCCTGACATGCAAAAACACTACCTGTTATTCACGCTGGTAAACCATCTTAGC |
| | | AATCAGGATGACTACGTGGAGAATGTTCAACAACCTTATGATATTAGCGAAGGTTTGTTTTTTCATCGGATCGCC |
| | | TCGACTTTCTTAAACATAAGTGGCATTCTCCGTAATATGAAATTCTATTCTTATCAGTCTAGCCGTCTGAAAGAA |
| | | CAGAGAGGCGAAACAAAAAGTGATAAAGGATATTTTGAGTGGATTGAACCGTTTCAGGGTAATTCATATTTTAG |
| | | CGTGGATGGTCAGAAAGGTGTGATTGGGGAAGACGAGCTGAAAGAGCTGTGTTATGTCCTCTTAGTAGGCAAGC |
| | | AGGATGTGAATAAAGTCGAAGGCCGTATTACTCAGTTTCTTAAAAAGTTCAAAAATGCCGACTCCGTCGAGAAA |
| | | GTGGAAGATGACGAAATGTTGGACCGGGAAAATTTTCCGGCTTCATATTTTGCGAAAACTCGTGTTGGCTCAATT |
| | | AAAGGGAAAATCCTGAACCGATTGAAAAAGGCCATCGAATCAAAAAAAATCACCGGGGTGGATGTAAAAGCAT |
| | | ATAATAAGATGAAGGAAGTGATGGCCTTTATTAACAACAGTTTGCCTGTCGATGAAAAATTAAAACCGAAAGAT |
| | | TACAAACGTTATCTTGGAATGGTACGGTTTTGGGGTCGCGAAAAAATCAATATCGAGCGTGAATTTCGTAGCAA |
| | | AGGTTGGTCGAAGTATTTTACGAGTGGATTTTGGATGGCCAAAAATCTGGAAGAAGCTTATAAACTTGCGCGAC |
| | | AGAAAAACGCTGAACTTTTCAATAAACTGAAAACGGCAGTGGAAAAAATGAACGAGCAGGAATTCGAGAAATA |
| | | CCAACAAATCAATGATGCAAAAGATCTGGCTTCTTTAAAAAGATTAGCGCAAGATTTTGGTATGAAATGGGAAG |
| | | AAAAAGATTGGCAGGAGTACTCTGGTCAAATTAAGAAGCGTATTACTGATCGTCAGAAATTGACGATAATGAAA |
| | | CAGCGTGTTACCGCCGGTTTGAAGAAGAAGCATGGGATTGAAAACCTTAATCTGCGTATCACAATTGATTCAAA |
| | | CAAAAGTCGCCGCGTGGTCCTGAACCGAATTGCAATTCCCCGCGGTTTCATTAAAAAACACATTTTAGGCTGGC |
| | | AGGGATCTGAAAAAGTTTCAAAAAAAATTCGTGAAGCGAAGTGTAAGATCTTGTTATCCGGAAAGTATGAGGA |
| | | GCTGTCCCGCCAGTTCTTCAAGGCCAAAGACTACGATAAGATGACCCAGATTAATTCCCTGTATGAAAAGAATA |
| | | AACTCATCGCGTTAATGGCTGTTTATCTCATGGAGCAACTCAACATACAACTGAAGGAATACACAGAGCTGGGT |
| | | ACACTGAAAAAGACGACTGTTGACTTCAAGATATCCAATAAAGTAACGGAAAAAATACCGTTCTCTCAGTACCC |
| | | AAGTCTGGTTTATGCAATGTCCCGTGAGTATGCGGACAATGTGCATGACTATAAGTTTTCTGAAAAAGATAAGC |
| | | CTTTTTTAGGAAAGATAGATGCCATCGAGAAAGAGCGCATGGAATTCATTAAACAGGTCCTGGAGTTTGAGAAG |
| | | TACTTGTTTGATAATTCCATAATTGATCGAAAGAAATTCGCTGATACAGCAACCCATATAAGCTTCAAAGAAAT |
| | | AGTTAACGAGCTGGTCAATAAAGGCCGCGACGGGAATGAATTAACAAAACTTAAAGATGCACGTGACACAGCA |
| | | CTCCACGGCGGAATTCTGACCGGCACTAGCTTCGAAGAAACGAAAAGCCTTATCAATGAAATGAAAAAATAA |
| 338 | JW13163 | ATGTGTAACGAGATTCAGACTAACCAAACCGGGCAGAAAGACAATTCGCTTTTTGGCATCTATCTGAATCAGGC |
| | | TCGGTTAAACGCCTATATCGCATTGTGCTACATTTCAGAACTCCTGGGAGAAAAAACAGTGGATGAAGATTCAC |
| | | TGTCTGAAATGCCTGTCTTAAGATTTTTGGATTCTGACAAAGACATTGTGAAAAGTAAGCGCACCTTCGAACTGT |
| | | TAGAAAAACGTTTTCCTATGTTAAAAGTTATCTATGATGGCGAACCAAAAAAGGAACAGGATGACAAGTTCTTA |
| | | GAACGCACAAAAAAATACAAAGAGATTCTTACCTGTCTCATTAAAGCGTTAAACGACAAACGAAACGAATTCTG |
| | | CCATGCCAATACAAAAGGCGGCGGCGGGGAATACGACATTAAGCAGTTGATTCGGTACCTGAATAATTGTTTCG |
| | | ATGCGAGCGTTAGACGGGTGAAAGATGTGCGTGGGTTAGAAGAAGAAGACGTTTTTCATCTGCGTAGAAAAAC |
| | | GTCACAGGGTAAAGGACGCGAAAAGCGAGTGATTGACAATCCGAATTTCTACTATCACTTCCAGGACAAAGAG |
| | | GGCAAAATCTCTGAGAAAGGTTTGGCATTCCTTGCTTCTGTTTTTTTGGAAAAAAAGGATGCCCATGAGTTATTG |
| | | AAAAAGCAGCGCGGTTTCAAAAAAGATGATACCCCAGGATATAAGGCGACACTCGAAAGTTTTTATTGTTTTCG |
| | | GATAAAACTGCCTAAGCCCGTCATGAAATCAGACGAAGACAAAAATGGACTGGCTCTGGATATGCTCAATGAA |
| | | CTGAAAAAATGCCCGAAAGAAATCTTCGATCTCCTGCCAAAAGAACAGCAGGAAGAATTTCGCGTCATAGATAC |
| | | CAGCGATGATAATGAGGATGATAATGAAATTCTTATGAGACGCTACTCTGATCGCTTCCCGTACCTGGCATTACG |
| | | TTATTGCGACGAGAATGAGTTGTTTGAAAACCTTCGCTTTCAAATAGACCTGGGTCGTTATTATTTCAAATTCTA |
| | | TGAAAAACAAACCATCGACGGCAACACTCAACAGCGGTCCCTCGATAAAAAGCTGAAGACATTTGGTCGAATC |
| | | AAAGAAGTGAAAGACAAAGTTCAACAGCAATGGCAGGATGTTATTAAAAGTCCTGATGAGATAGAGGATGAAC |
| | | AAAAAGAGCCTTACAAAGCTAATACGACGCCTCATTATAATCTTGTGGACAACCAGATTGGTTTTGTGATGTCC |
| | | AGAGATTGTAATCTTCCAGATCTGAATCAACCTGATGGCAAACTGAAATTACAAAAACCCGATGCTTGGCTGAG |
| | | CATCTATGAATTACCGGGAATGATTTTTCATGGTTTGACGAGCGACTTCAAACAAACTGAGATTTTAATCAACAA |
| | | GTATATTGGTCGTCAGCGGTCGATTTGTAAGAAAATTTTCGGCACCGGTAGCATCCCGCAGGATGCTGGTGAAT |
| | | ATCTGCCAGAGGCACTCAAAGATATTGAAGAGAATGATACTAAACGGACCTCGTATGCGAAAAAAAAGTTATG |
| | | CAGAATCCTTGAAGACACAGAACAGCGGATAAAGGCTATTAAAAAAACCGAGCAACGTCTGGTCGATAAAAGT |
| | | AATAAGCCCGGTAAAAAGCAGTTCTTTGATATACGTGCAGGCAAACTTGCAGATTTTTTGGCTCGTGACATAAT |
| | | GATATTACAATCATTTGATCCGGCCAAAGATGGTAAAGATAAACTGACATCTATCAACTACCAGGTTCTGCAGG |
| | | CAACCCTCGCATATTATGGCGCGAAAAAAGATAGCATCGCCGACATGTTTAAAACTATTGGATTGTTAGACGGA |
| | | GATAACCCTCATCCTTTTCTGAAAGAAATCAATCCTTCGAAGTATAACTCAATTGTTGAGTTCTACAAAGCCTAT |
| | | CTGAATCGAAAGAAAGGGTACCTGGAAAAATGTGAAAAAGAGGGTAAGTTTGATGAACAGTTCCTGCGTCCCA |
| | | GCCGCCAGCGTTATGCAAAAGGCAAACGAGATCTGAAAACTATCGCGAAACAGCTGTTAGATAACCCGGTCAA |
| | | TATTCCGAAAGGGTTCTTTCAGAAGCAAATCAAAGAATTTGTATGCGTACGTGAACCATCTTTGGAAGAGCTGC |
| | | AGATGAATACTGCCTATATGATTCAAGCCTATTTTGAGAAGGAATACGGGACGCAGCAGCCGTTTTGCTCCTATC |
| | | AAAAGACATATCCGGTAGTCTCCAAAAGTACGGAGTATTGTAAAAAAGCGAAAAACAAAGAGATCAAGGAAGT |
| | | CTTGCGCAAAATTACCGCAGAAATGTCATATCGGGAACTGCGTAAGTTTATTGATAGTAAAATACCGGACGGCC |
| | | GTTATGAACCAGAGAATCTGAAAAAGAATCTTCTGGATGGGTGTCGTGACTTCAAGAACAATGAGCGCCTCTTA |
| | | CGCCGTTTTAAGGTACAAGACATGGTAATGTTTATGATGGTTGAAAAAACTTTAAAAGATCAACTGGCGTTTGA |
| | | AGGTAATGTAATTAAGCTTAAGGATATTACCCCAGTGGAAGATTCTCCCTTTAATAAGCCCGTGTTTTGCAGCTC |
| | | AGACGTCTCCGCTAGTTTTAATACCAACAAAAAGTACGAGAGCGGATACGTTGACTTCATCAAAAAAAACTACA |
| | | AAGGCCTCTACATTTCCCAGGGCGGTCGCATTATATTGAAATATCGCGTAACGTCTGAATCCACGAAGCTTAAA |
| | | GATATAGGTAAGTATAGACGCTATCTTTATGATCGAAGACTGCCGGGATTACTGATATGGAAATATCCGCCAAA |
| | | TATTGAAGGGGGTATAGAGATTAAGTATGCCGACATAGAAGAAGAAATTAAAGCGTACGAGAAATACCGTAAG |
| | | CAGATTGTGCAACAGATCTACATGCTGGAAAAAAAAGCCGTTGATCACTTTAAACTTGAGATTGAGCCGGAAGA |
| | | AAATCATATCCCCTTTAATACTGTGACGGATACTATTAAGTTTCGTTTGGCAGGGAGCGAAGAGAAATGCGATA |
| | | CACTTCTGAACATCCGCAACTCGATAAACCACAACCAGTTCCCGGTTTTTGATGAAAAAATAGAGAACGCCCAG |
| | | GGACAGAGTATCAGTGAACGTATGTTTTCAATCACGGAGAATTATGTAAAGTATATTACAGAGCACATCACAGA |
| | | GTCGGATAAGGACTTTGCGTATGTTTAA |
| 339 | JW13164 | ATGTTATGTCCGCGCGCCATCTTTTCGACCTTATTCAACTTTGTACACATGGAAGCAAAACACACCATACAGTAC |
| | | TTTGACTTCACCAATATCAAGCATAAACATTTCTTTGCAGGTCTGTTAAACACCGCTCAACACAACTTTGATCTT |
| | | AGCTTACGTGAATTAGCAAAAAGAATTCACAATAAGGATACGACCAAAGGGTTGATTACAATTGCCTTTAATAA |
| | | TTCGGTGTCTGAAAGCGATTTCAAAGCCCGTACTAATTACTTGTCAGAAAGTTTGGCGTTCATTAAACTGATGAA |
| | | CTATGAGAATCGTGCGGAATTCCGCGACACACTGCTGGTGCTTTATAATACAATTGACGGGCTGAGAAATTACT |
| | | ATACACATTATAAACGCGATAAAATTACGATAGACGAGTCTGTATACAAGCTTCTCGATTATCTTCTGCTGACAT |
| | | CAGCAAAAGAAGTTAAAATTAATCGCGCGAAAAGTGAAGAGTATAAGAAGTACCTGAGCATTAAGTATCAGGA |
| | | CGAATTTGAAGAGATTATTGACGAGAGTAACAAAAAAATTGAGGAAGATAATGCGCTGTTAAAAAAGGGGGAA |
| | | AGAAAAACTGGTTTAATTAAGCCACAAGATGAAGTTAATCATGTCCTGAATAAAGTCTTTCGGTCCTTTCTGTAT |
| | | AAGAATGATAAAGGGGAAGATGAACTGCGCGATAATGCTAGCTCAAAAAATAAAGAAGACACAGATTTGAGTA |
| | | CCTATGGCTTTGTACAACTCTTAGCCTTGTTCCTGAACAAAAAACAAACAGAATCTTTGCTGTCGTATACGAAAT |
| | | ACCTTAAAGACACGAGAAAACTGAAATTCGTAACTTCCCGTTGGACCCTGACCTTTTTATGTTATAAGGATATTA |
| | | AACGGTTATTCAAATCCGACTACTCCAATGATTCTCTTCTTCTGCAAATGATCTCTGAACTGACCAAGTGCCCAA |
| | | ATGAAATCTATCGACAACTGACTCCTGATAAAAAACAGGAATTCATAGAAGACATTAACGAGTATTATAAGGAC |
| | | ACCATATCTTTTGAAGGAACCAACGAACAAGCTCTCGTAAGTCATGAAGTTATTCAGAAGCGTTACGCTAATCG |
| | | CTTCAGCTACTTCACGATTCGGTTTCTGGATGAGATCATTGATTTTCCCACACTCAAATTTCATGTTAATCTCGGC |
| | | AAGTTTAATCATGACACTACCAAAAAGAAGTTTTCTAACGCCATAGAGACGGAACGATCAATTTTAGAAAAAAT |
| | | TACCGTGTTTACAAAGCTTAGCACGGCAACGAATAATAAAAAGGACTATTTTTCCAAACCGGAGAATAAAGAGT |
| | | TGGAAAACGATTGGTTAGAGTTCCCCATGCCATCATACCAGTTCAACAAAAACAATATTGGTATATGGCTGTCA |
| | | ATCGATAAAAAATCCGGAGAATCCGACGCCCCAGAAGAGCGGGAGAACAGTAAACTGAGCAAGTATAAGATCG |
| | | CGGAAAAATTGTTACTTAAAAATACTATTCGGAAACCGATTGCGTTCCTGAGCTTCAATGAACTGCCTGCCCTGC |
| | | TGTATTCACTGCTGATCGAAAAGAAAACTCCGATTGAGATAGAGAAGATTATACAGAGTAAGATAATCGAACA |
| | | GCGTAAACTTATTAGCTCATTTGACAAGACTAAAATTATCTCGAACAAACGTTTACCGAAAAAATTAATACGTG |
| | | CGTCTCATCTTGATTCAACCACGGATGTCAAAAAATTGGAAAAAGACATCAAGACTGAGATAAGTAAAGATCCG |
| | | ATCGGTGATATTCGTAAACATTATAAGAAGGAGATCAAAAATGTGAATGAGTTCACTAATAGTGAAAAAGGTA |
| | | AAGTTACGACCTGGCTGGCCGATGATATCAAACGTTTTACCAGTAAAGAGACACGCGAAAATTGGAAAGGATAT |
| | | CAGTACGCAGAATTTCAGGCATTACTGTCCTTCTATGACATCAACAAAGACAAACCTAAGAGCTTTCTGGAAAA |
| | | AGACCTTAACTACGACTACAAAAAAGATAACTCCTTTAACGGCCTGAACTTTAATCTGAAAACCCTCTTCGATTT |
| | | TTACAACTCTTACCTTAAAAATCGCCGGGAGTACCTCAACAATTTGTTAAAAGAAATCGAGTCGTCTAAAGATG |
| | | AAATTCATGAAAAAGTGTTTATAGGTTTCACGAAGAGATTGTATGAAATCAAACCGATCGACATCCAGAAGAAT |
| | | GTATTTCTCAAAAAAATCCCACTGAACCTGCCTCGCGGAATTTTTGATGATAAACCGACTAGTTATGATAAAGGT |
| | | GGTAAGGATAATACAGAAATTGCTTCGTGGTTTACACTGAGTAGTAAAGCTGAAAATGCTCAACTGTATTATTG |
| | | CTTCCCTAAAGTGTATTCTTATCCCACAACAAAACTGAAATGGGATAATAGAAAACGTCGTAAAATACAGAAAT |
| | | CGAGCATCGCAACTCAACTGATCTCACCCAAAGAACGCCTGAAACAACAGTATTTGAAAGATCTTCCGAAAACT |
| | | GTGCAGGTTGAAATCTACAAAAACGAAAAGTCAATTCGGAAGATTATGAGAGAGGATTTTTTCATTTTAGAAAT |
| | | GGTCAAGCACTTGCTGAAAAAGTCAAGCATTAAAGGCCTGGATAAACTCCAGTTAAAGGATGTTTTTTTAACAA |
| | | AAGAAGAGAAGAACCGCCGTATAGTAGAGAGCTTAAAACAGTCTCAGCGTGTGAAAGGAGATAAAATGGAGA |
| | | ACGTGGTTAATGACTCTTATATTTTATCCCAGCAGGTTGCGATATCTATTCTGAACGACAAGATAATCGACACCG |
| | | TTCAGATCAAGACTATTGGCAAACTTCGTCGATTAGAGGAAGATAAACGAGTCCGTCAACTGATCGAATATTTT |
| | | CCAGATAAACCTTCGTGGACGGTTTTAGAGATTCAATCAGAAATTGATGATTATGATCGGGTGCGAAAGTTTGA |
| | | ATTTTTTAGCTCCGTCCACGTGCTGGAGAAGCTTATCTATAAAGATGCGAAGAAAAATAATGCCAAAGAGGGCC |
| | | TTCTCCAGAATAATAACCATAATTTTCGTAAGTACTTGAGCTATTACTATCTCAACGAAGATCGTGATGCTTTTA |
| | | ACTCATTACCGAATTTCGACACCATAACGATAGATAACCCTTCAATCAAAAGTGAATGGTATCGCCTGTATCTTT |
| | | TGGTTAAGATTCGCAACAAATTTTCCCACAATCAGTTATTGAGTAAAGTCGATTTTGACTCGTTAAAAGAGATTA |
| | | TCAGCATGAATTCTGGCGAAAAAATTGCCGCATTTCTGAACCGCGCATTTGATAAACTCATTATAGTCAAATAA |
| 340 | JW13165 | ATGAACAATAACGCGACTATTCAGAAACAGTCTGTTATCTATGCATTCGACAAAAGTGAGGACAAACACTTTTT |
| | | CGCCGGGTTGTTAAACATGGCTGAGTTAAACTTAGTTATCGCCCTGCAAGAGACCCTGATTCGGACCATTCGCA |
| | | ACACGAAAACAAGCAACAATGCGAAGGTACTTATCCAACAACTGTTCACTGAAGACATCATGCAAAGCGATTG |
| | | GAATTATCGGGTGGAAGTCCTGAGTGAATTTTTGCCCGTTACTAAAGCCCTGCAGCCACAGGCCGATAAAGCTA |
| | | CGTTTATTAAAACATTGACCCTCTTTGCCGAAACGCTGAACACCTTACGCAATTTTTACACTCATCATTACCACC |
| | | CGCCTGTGGAGATTGATTCAAGACTGTTTGAGATTCTGGATGCCGTACTGCTGCATAGCGCGGTTTATGTTAAAA |
| | | AACAACGCCTGAAAACGGAAGATGGCAAAACCATGTTTCCGAAACAATATGAAGATGATCTTCGTACCAAACTT |
| | | GCCATACATAATGAAGGGGTGAAGCAGAAACGCATAAGCGAGAGAGAAAAACCGGAAAATAAAAAAATCCCG |
| | | AAGAACAAGTTCAACGAAATCTTCAACAAAAAGAACAAGACTTACAGTGAAGCAGATTTTGTGAATACATATCT |
| | | TAATGGGGTGTTTCACCATTTGTTATACAAAGATCGTAAAAAGGAAGGTTTGGACCAGCTTAAAAAAGATGCCG |
| | | CATCCAAGAAAAAGGACGAGAATTATTTTAGCAAAAACGGATTCGTCCTGTTCCTCTCGTTTTTCCTTGACAAAA |
| | | AAAATCTTGAAGACCTTTTTTCTCATACAGAACATTTCCGCGGCACCCACGAATTACGCTTTATGGTTACACGTT |
| | | GGGTGTTTTCTCTGCATGCTTTTCGTGATGTCCGTCGTGTATTACGCTCAGATTACTCGAATGATGCCCTGCTTCT |
| | | GCAAATGATAGGAGAGCTGAACAAGTGTCCAAAAGAATTGTTCAATATTCTCTCGACAGAACACCAGCTGGAAT |
| | | TTTTCGAAGATCTTAATGAATATTACTCTAATAATGAAGGCGGAGATGAATCTCAGCCGGATTCTTTAGTAACGC |
| | | ATACCGTGTGGCGGAAACGCTATGATGATAAGTTTTCTTATTTCGCGCTGCGATTTTTAGATGAGTTCGCTGGCT |
| | | TTACGAACTTGAAATTCCAGGTCGTGGTCGGTAATTATGTCCATGATTCCCGTACAAAAGAACTCGCAGGCATC |
| | | AATAAAGTAACCTACCGTACGATTAAAGAAAAGATCAAAGTGTTTGGTAAACTGAGCGAGATTGATCGGCTGA |
| | | AAAAGGAATATTTTCTGAAAAAGAATCAGGAGCAGCAGGTTTCAGATCCAACGACAGAAACAACCGAATTAAG |
| | | CGAAGGTTGGGAAGAATATCCTGTGGCACACTATAAACTGGATAACAATAATATTCCGATCTATCTTAGCATTC |
| | | CAGGTTTTCAGAAGGATAGCACGCCTAAAGAATATCGCACCAAACGCCTTAACAAAAAATCTATTTTAGAACAG |
| | | CTCACTACCACCGGCTTTATAGAATCTGAGCCTATCGCCTTTTTATCGGTCAATGAGTTGCCCGCGCTCTTGTATC |
| | | AGCTCCTTTTCAATAACAAGAGTGCGGCACAGATAGAGACCATGCTGATCCAAAAAATCACATCCCAGGTGTCA |
| | | GAGATCAAGCGGTTCAACACGGAATCAACTATTGATAAACATAAAATTCCCAAGAGACTGATCCAGTTAGCCAG |
| | | CACTATAGCTACTTCCACAAGTATCAATTTTGTTAAACTGTTGAAAGACATCAATTCGGAGATTGATACGTGCAA |
| | | TAGCCGTCTGAAAGAGACGGATGATAACCTTAATAAAAGCAAGTATAGACGCTTTAATAACAAACAGCGCGGC |
| | | AAAGTTGCGACTTGGCTGGCAGACGATATCAAACGTTTTGCTGGTAAAGATATTCGATCTAATAATTGGAAATC |
| | | TCATCTGCATGGTGAACTGCAGGCTCTCTTAGCATTTTACACTGAGCGTAAAGCGGACATCCAGTTACTGTTGGA |
| | | GAAAGAACTGGGACTGCCATTAGACAGTAAAAGTTTGCTGTCAACCAGCTTTTCCGAAAACACGTTAGAAGGAT |
| | | TTTATGTAAAGTATCTCAAGACTCGTCGCGAAATACTCACTGACTTTAAAGCATCCGTAGATAAAACAAAAAAT |
| | | GAGTATCCCAATTCATTGGATACATCCACCATAGAGCCACTGTTTGACATTTTTGATAAGCGCCTCTATGTAAGT |
| | | TCACACATTGAGAAATATAAGTCAGACCTGTTACAAAAACCTCTGAACCTGCCTCGAGGCATATTTGATACCGC |
| | | GACTTATTTTCCACTGCAGAAAGGAGATAAAAGTAATTTTCAGCCTTGGTTTGAAGTTGCGGCTAAGACAGAAC |
| | | ACTACCAAGAATTCTACAATTTTCCGAGAAAGTATGAAACAATTGAACTTAATCCGGTGTCCGGTAAAGGGCTG |
| | | TTAGAACAAAACAATAATAAACCGGTTGATCCGGACGTGTTTAAGAATGAAAAAAGATTACGGGAAATTGCGC |
| | | GACAGGACTTCTATGTTCTGGAAATGGCAAAGTACATGATTAAGCACATGTATCCCGCTATTCGTACCAGCCAA |
| | | AATTACACGTTGAAGGACGTGTTCAAAACAAAACAGGAAAAAGAACAGATAAAACTTCGTGCAGCCCAACAAT |
| | | CCCAGAAAAAAGCGGGAAGTGGTGGTGAACATTTTACAGATGATTCTCATCTTCTGTTACGTCGTTTTCCGGTAA |
| | | CCGTAGCTATTGGTAACAAAAAAATAGTAGATTATGTTGCACTTAAGGACATTGGCAAGTTTCGGAGATTGGAG |
| | | AAGGACAATCGTATAAAGCTGTTATTATCATATTCAGAGCAAGAAATTTGGACCGTGAAAGAGATTGAGGATGA |
| | | GATAACGAACTACGAGCATATCCGTAAACAATCGTTTTTCAAAGAAGTGCATGTCTTCGAGCAGGAAGTGCTGC |
| | | AGAAAGTTAAGCTGAAAGCCGGTATAGACACTCACCCAGAAATTCTGGAACAAAATGGCTACCCTAATTTCAAA |
| | | CACTACGTCGCTTACCATTTTTTTAACAATGACACCATCACCTTCGACAAGTTTGTCAAAGTTGATTTCCAGAAC |
| | | GAGAAAAGTCGGAAAGAAATTGTTGAGTCAAAAAACATGTTATTGCTCAAAGCGTACTTCTTAGTCTTAATTCG |
| | | AAACAAATTCTCCCATAATCAGCTCCCGGAGAAGGTTGATTTCCTTAAAATGAACGCATATATCAAATTTTCGGG |
| | | GGAGAAGATTGGGGACTATCTGCTGCATTTTTTTAAAGAAAGTATTGTTTATTAA |
| 341 | JW13166 | ATGAGCGCTATGACTAAAGGGCTCCGTAACTGTAAGGGATGTGTTAATATGAAAAAAAAGATGAGCTTACGGG |
| | | AGAAGCGGGAAGCTGAAAAACAAGCCAAAAAAGCGGCTTATAGCGCCGCCAGTAAAAACACGGATAGTAAAC |
| | | CTGCGGAGAAAAAAGCAGAAACCCCAAAACCAGCAGAGATAATTAGTGATAATTCGAGAAATAAAACTGCCGT |
| | | GAAAGCTGCAGGGTTGAAAAGTACAATAATTTCTGGAGATAAGCTGTACATGACCAGCTTTGGGAAAGGCAAC |
| | | GCTGCCGTTATCGAACAAAAAATCGATATCAACGACTATTCGTTTTCCGCGATGAAAGATACGCCGTCATTAGA |
| | | AGTCGATAAAGCTGAATCTAAAGAGATTTCGTTTAGTAGCCATCATCCGTTTGTCAAGAATGACAAACTGACGA |
| | | CCTATAATCCTTTATATGGTGGCAAAGACAATCCTGAAAAACCTGTAGGCCGGGACATGCTGGGTTTGAAAGAT |
| | | AAACTTGAGGAACGCTATTTCGGGTGCACGTTTAACGATAACCTGCACATCCAGATTATCTACAATATCTTGGAC |
| | | ATCGAAAAGATATTGGCTGTTCATTCCGCAAATATTACAACCGCTTTAGATCACATGGTAGACGAGGACGATGA |
| | | GAAATATCTGAACTCTGACTACATCGGCTACATGAATACGATAAACACTTATGACGTTTTTATGGACCCGTCGAA |
| | | AAATAGCTCGTTATCACCCAAAGACCGTAAGAATATTGACAATTCGCGAGCAAAGTTTGAAAAACTGCTCAGTA |
| | | CTAAACGACTCGGCTACTTTGGGTTTGATTACGATGCGAACGGAAAAGATAAAAAAAAGAACGAGGAAATCAA |
| | | AAAGCGCCTTTATCACCTCACAGCCTTTGCCGGACAGTTGCGACAATGGAGCTTTCATTCTGCGGGTAATTATCC |
| | | AAGAACATGGTTGTACAAACTGGACTCATTGGATAAGGAATACCTGGATACCTTAGATCACTACTTTGACAAAC |
| | | GCTTCAATGACATTAACGATGATTTTGTGACAAAAAACGCCACCAATCTCTATATCTTAAAGGAAGTGTTTCCGG |
| | | AAGCAAATTTCAAAGATATCGCGGACCTGTACTACGATTTTATTGTCATCAAGAGTCATAAAAACATGGGCTTC |
| | | AGCATAAAAAAACTGCGTGAAAAAATGCTCGAATGCGATGGCGCAGATCGCATCAAAGAGCAGGACATGGACA |
| | | GCGTACGCAGCAAGCTCTACAAACTGATAGATTTCTGTATTTTTAAGTACTATCACGAATTTCCGGAATTGTCCG |
| | | AGAAAAACGTTGATATTTTAAGAGCGGCTGTATCTGATACTAAAAAAGATAACCTGTATTCTGATGAAGCCGCA |
| | | CGTTTATGGTCAATCTTCAAAGAAAAATTTTTAGGTTTCTGCGACAAAATCGTTGTGTGGGTCACAGGCGAACAT |
| | | GAAAAAGATATTACGTCAGTGATTGATAAAGATGCATATCGCAACCGCTCAAACGTTAGTTATTTTTCAAAGCTT |
| | | ATGTATGCAATGTGTTTCTTTCTGGACGGAAAGGAGATTAACGACTTGTTGACGACCCTGATAAACAAGTTTGAT |
| | | AATATTGCCAACCAGATTAAAACCGCCAAAGAGCTGGGTATTAATACAGCATTCGTTAAGAACTATGATTTCTT |
| | | CAATCACAGTGAGAAATATGTAGACGAGCTGAACATCGTCAAAAATATTGCAAGAATGAAGAAACCATCCTCA |
| | | AATGCCAAGAAAGCCATGTATCATGATGCTCTGACAATACTGGGTATTCCCGAGGATATGGATGAAAAAGCATT |
| | | AGATGAGGAGCTGGACCTGATTCTGGAAAAAAAGACCGATCCAGTGACGGGTAAGCCGTTAAAAGGTAAAAAT |
| | | CCCTTACGGAACTTTATCGCGAACAATGTGATAGAAAATTCACGTTTCATCTACCTTATAAAGTTCTGCAACCCT |
| | | GAAAACGTGCGCAAAATTGTTAATAATACCAAGGTCACCGAGTTTGTTCTCAAGCGCATTCCAGACGCCCAGAT |
| | | TGAGCGCTATTATAAGTCTTGTACGGATTCCGAGATGAATCCGCCGACAGAAAAAAAGATTACCGAACTGGCGG |
| | | GGAAACTTAAAGATATGAATTTCGGTAATTTTCGTAATGTTCGTCAGTCAGCCAAAGAAAATATGGAAAAAGAA |
| | | CGCTTTAAAGCTGTAATTGGTTTATATCTTACAGTGGTTTACCGGGTGGTAAAGAATCTTGTCGATGTCAATTCT |
| | | CGTTATATTATGGCTTTTCATAGCTTAGAACGCGATTCTCAGCTGTACAATGTATCCGTTGATAATGATTATCTTG |
| | | CACTTACAGATACTCTGGTGAAGGAAGGTGACAATAGTCGTAGTCGTTATCTTGCAGGAAATAAACGGCTGAGA |
| | | GATTGCGTCAAACAGGATATAGATAATGCGAAAAAATGGTTCGTTTCAGACAAGTATAATTCCATTACTAAGTA |
| | | TCGTAATAATGTGGCCCATTTGACTGCGGTGCGTAACTGTGCGGAATTCATAGGCGATATTACCAAGATCGATA |
| | | GCTATTTTGCACTGTACCACTATCTTATTCAGAGACAACTGGCGAAAGGCCTGGATCATGAACGATCTGGATTTG |
| | | ATCGTAACTATCCGCAATATGCGCCTCTCTTCAAATGGCATACTTATGTAAAAGATGTTGTGAAAGCACTGAACG |
| | | CTCCCTTTGGGTATAATATTCCGCGGTTTAAAAATCTTTCTATAGACGCGCTGTTTGACCGGAACGAGATCAAAA |
| | | AAAATGACGGTGAAAAAAAATCCGATGATTAA |
| 342 | JW13167 | ATGTCAAATGAAATTGGCGCCTTTCGGGAACATCAGTTTGCCTATGCTCCTGGGAATGAAAAACAAGAAGAAGC |
| | | CACATTCGCAACGTATTTTAATCTTGCGTTAAGCAACGTGGAAGGTATGATGTTTGGAGAAGTGGAATCAAATC |
| | | CCGATAAGATTGAGAAAAGCTTAGATACGCTGCCGCCTGCAATCTTGAGACAGATTGCATCTTTTATTTGGCTGA |
| | | GTAAGGAAGATCATCCAGATAAAGCCTATTCGACGGAAGAAGTTAAAGTTATCGTGACTGATCTGGTTCGTCGT |
| | | TTGTGTTTTTACCGCAACTATTTTTCTCATTGCTTCTACCTGGATACTCAGTACTTCTACTCCGATGAACTCGTTG |
| | | ATACTACAGCTATCGGCGAAAAGCTGCCGTATAACTTCCACCATTTTATTACCAATCGGCTGTTTAGATATTCAC |
| | | TGCCTGAAATAACCTTATTTCGCTGGAATGAAGGAGAGCGAAAGTATGAAATTTTGCGCGATGGCCTCATATTTT |
| | | TCTGCTGTCTTTTTTTAAAACGCGGACAGGCAGAGCGCTTTTTGAACGAGCTGCGTTTCTTTAAACGGACTGATG |
| | | AGGAAGGTCGGATTAAGCGTACAATTTTTACTAAGTATTGCACCCGTGAGAGCCACAAACATATAGGTATCGAA |
| | | GAGCAGGACTTTTTGATTTTCCAAGATATCATTGGTGACCTTAACCGCGTACCCAAAGTGTGCGATGGTGTGGTT |
| | | GATCTTTCTAAAGAAAATGAGCGTTATATCAAAAATCGTGAGACATCGAATGAATCAGACGAGAACAAAGCAC |
| | | GCTATAGATTATTAATAAGAGAGAAGGACAAGTTCCCTTATTATCTTATGCGATACATCGTGGACTTCGGCGTTC |
| | | TTCCGTGTATTACCTTTAAGCAGAACGATTACTCAACCAAAGAAGGAAGAGGGCAGTTTCATTATCAGGACGCT |
| | | GCCGTAGCCCAAGAAGAGCGGTGTTACAATTTTGTAGTTCGTAACGGCAATGTGTATTACTCCTATATGCCACAA |
| | | GCTCAAAATGTAGTGAGAATTTCTGAGCTCCAGGGCACGATAAGCGTAGAAGAACTGAGAAATATGGTGTACG |
| | | CTTCCATCAATGGGAAAGATGTCAACAAAAGTGTCGAGCAGTACCTGTATCATCTCCACTTGCTGTATGAAAAA |
| | | ATCCTGACCATTAGTGGTCAGACGATTAAAGAGGGAAGAGTGGACGTTGAAGATTATAGACCTCTCCTGGATAA |
| | | ACTTTTACTGCGGCCGGCGAGCAACGGGGAAGAATTACGACGCGAGTTGCGCAAACTGCTGCCGAAACGGGTTT |
| | | GCGATCTGCTCTCTAACCGTTTTGATTGTTCTGAGGGAGTCTCGGCAGTAGAGAAACGCCTGAAAGCGATTTTAT |
| | | TGCGCCACGAGCAACTGCTGTTATCGCAGAATCCAGCCCTGCACATCGATAAGATTAAGAGTGTGATCGACTAT |
| | | CTTTATTTGTTCTTTTCCGATGATGAAAAATTTCGACAACAGCCAACCGAAAAAGCACATCGGGGCCTGAAAGA |
| | | TGAAGAATTTCAGATGTATCATTATCTTGTTGGTGACTATGACTCACACCCACTCGCACTGTGGAAAGAACTGGA |
| | | AGCCAGTGGACGGTTAAAACCAGAAATGAGAAAACTTACCAGCGCTACCTCTTTACATGGGTTATACATGTTAT |
| | | GTTTAAAAGGCACAGTTGAGTGGTGCCGCAAACAGTTAATGTCGATTGGCAAAGGCACAGCGAAAGTTGAAGC |
| | | CATAGCCGACCGTGTAGGGTTAAAGTTATATGACAAGCTGAAAGAGTACACACCGGAACAGCTCGAACGCGAG |
| | | GTCAAACTTGTTGTAATGCATGGTTATGCGGCTGCTGCTACTCCTAAACCGAAAGCGCAAGCGGCGATACCGAG |
| | | CAAACTTACAGAGTTACGATTTTATTCGTTTCTGGGTAAACGCGAAATGAGTTTTGCCGCGTTTATCCGTCAGGA |
| | | CAAAAAGGCTCAAAAACTGTGGCTCAGAAATTTCTACACGGTAGAAAATATTAAAACGCTCCAAAAACGTCAA |
| | | GCGGCGGCCGACGCAGCCTGTAAGAAATTATACAACTTGGTTGGTGAAGTCGAACGCGTCCACACCAACGACA |
| | | AGGTGCTGGTTTTGGTCGCACAGCGTTATCGGGAGCGCCTCTTAAACGTCGGTAGTAAGTGCGCGGTCACACTG |
| | | GATAATCCTGAACGACAGCAAAAACTGGCAGATGTATATGAAGTTCAGAATGCATGGTTGTCCATTCGTTTCGA |
| | | TGATCTTGATTTTACCTTGACCCATGTTAATCTTAGCAACCTTCGTAAAGCATACAACCTGATTCCCCGTAAGCA |
| | | CATATTAGCGTTCAAAGAATATCTTGATAATCGAGTGAAACAGAAGCTGTGTGAGGAATGCCGCAACGTCCGTC |
| | | GGAAAGAGGATCTTTGTACTTGCTGTAGTCCCCGTTACAGTAATCTGACATCCTGGCTGAAAGAAAATCATAGC |
| | | GAGTCATCAATAGAACGTGAAGCTGCAACGATGATGCTGCTGGATGTGGAACGGAAACTGCTGTCCTTCTTACT |
| | | GGACGAACGCCGTAAGGCAATAATCGAATACGGGAAGTTCATTCCGTTCTCTGCGCTGGTGAAAGAGTGCCGTT |
| | | TGGCAGACGCCGGTTTATGTGGCATCCGTAACGACGTTCTGCATGATAATGTAATTTCTTATGCCGATGCAATCG |
| | | GTAAACTGAGCGCTTATTTTCCGAAAGAAGCGAGCGAGGCCGTCGAATATATTCGCCGTACTAAAGAAGTGCGT |
| | | GAGCAGCGTCGCGAAGAGTTGATGGCTAATTCTTCACAGTAA |
| 343 | JW13168 | ATGAAAGTAGTCAAACCATGTGGAAACTCAGTTGTGTTAGCTGGCGGTGAAATTCGCCGTACAATACAGCCGAG |
| | | CATCACCAGTATTACTTCTGTTAATGCAAAACAAACGCATAGACCGGCGATAGATTTTTTATTGAACAACGAGA |
| | | AATTTTTTTTGGCTCAATGGGCCTCAATGCTGGATAAAATAATCTCTAAACCGCATCAGGCAAATCAGGCTAGCC |
| | | CAAATTATGCCACCTACGAAGCACGCGAAAACCTCAGCAAGGCGTTCTACGTGCTGTGGAAAGACAAAAAACT |
| | | GCTTAACAATTTCTCACACAAGGACAACCAGCGAAAAAGTTTCAATCAACAGTGGGGACTGAAAGTACATCCAT |
| | | ATAAGAAACCTAATAAGCCCACCGCAAATAATAAACCGGAAGCTATTAAAAAAGCGGGAGAGATTTCCAAAGG |
| | | CAGATGGTATGATTCTTTAGTGACAAAAAGTAAACCTAACAAAAATACGGCCAAGCACCACAAAAGTGCCACG |
| | | ATTCACTATAAGAACACCGCGAAGATAATCTATGAGCATCTTTATGTAAAACAACGTAGTATCTCAAAAAGATC |
| | | AGCATATCCTCAGGGCTATATCGCCAATCGCGCAAACTCAATCGAAAAAAGCGTTCACTCGTTCACGCTCAAAG |
| | | ATAACAAGCTGGCAACTTTATGCATTAACGATCAGCACCGTATCAATAGACTGACAAAACAATCCATATCCATT |
| | | GCACAGCCGTCCAAGTATCGCCTCGATGGCCTGATCAAGGACCTCAAAATTAAAACGAAACAATATTACCAGAA |
| | | TAAAGTTTCGGCTGCAGAGTTACGCGCCAAAAAAACAAAAAGACCGGTAAAACCGCCGCTGGAAAATCAGCAG |
| | | TTGCCCTTAAAAGAAGCGATGATTTGTTTATATGAACACTATGCTCGGATTTTCGGGACCGTTACAAAAATTGAA |
| | | ACCATAATTATTAAGGAACCGGAATTGTGGCAATATCACTGCCTGATCAAGAGTACTTTAAAACAACGCTACCC |
| | | AAAACAAGCTGTGCTTAACAAAATCTACCCTGTCATCACAGAGGATCAGCTGCATAAACTTGCTCAGAATATAG |
| | | AGACAAATAAAACCCTGGCGATGGCGATCCGGTTGGGCAAAGTTGTCCATTATTCCCAAGACAACCTGGGTTAT |
| | | GAAGATATAGAACACAGCGATATTGTTAATTCTGATTACTGGGGTAGCGCGGGGCAGACCTTAATTAAACAGAA |
| | | AGAAAGCTTCGCACAACATTGGTTAATTGCTTTATCCGTCGCCAATGGGAGCCTGCGTGGGCTGTTAGATACTCA |
| | | AGATAATGCTGATATCTATCTTAGCCGTGGCATTAACACCGCGCTGGAAGGCATTCCAACGGATTTCGAGGCTC |
| | | GTTTTAAACTGCTGTTTGGTAATCACCATCAGTTGTATCAGGATGATCTTCAGCGGGATGAATTGTTACGACAGT |
| | | TATACGGAACGTTATCTCAGCTGCGTCACGCGTCTTTTCATTTTAAAAGTGAGCAGTCCTTCAATCAGGTGCTGT |
| | | TAAAGAAAGTGACTTTAGACACAACCCTGGATAATAACACCTCGGTTTCTCAGACACTGGCCAAAAACATCAAC |
| | | AATTACATAGACGAACACCTTGCACAGCAGCAAGCGCTCGTTAAAAAAACATTAGAAGCAAGTTTCGTCACAAA |
| | | GTACCTTAATCAGACCCAGTGTGATCAACTCTTATCGTCACTCCAGAACAACCAAGCAATTGCGACTCTTCTGCC |
| | | TAAGTTTAATAAACTTCTGCATCGCGCCGCCACTACCGAACAGTCGATAATTGCTGTGGCCAATCAGGTCGAGCT |
| | | GCAGAATAATCCGAATCAGCAATGCGCCTTTATCTGTCTGAAACAGCTGTATGAACAAGACTTCTGGCGTTGGTT |
| | | ATTAAAACAGAAAAGCAGCACTCTGAACCATCATATTGACCAGGCAAAGATTCGTGCGAACAAGCAGGCCAAC |
| | | AAACTTAATGCCAAAAATACTCTCGAGCATCAAAAAATAATGGCGCGTATGCAACAGTTGCGTGGTATTAATGC |
| | | CCAAGAATCATTGTTAGCTTATTTTAGCTATCTTTCTGCACAAACGGCGTCATTTTTTCAAATTCAGACCAACACT |
| | | TATCAGCATAACGCCCAAGCAGCCTCTGAACATAGCAATTTTATCGAACAGCTGAAACAGGACTTCTTGATTCG |
| | | CGCGTTTAAGCAATATCTTAAAGCAGAGAAGTTTAACTATTTGTTACATCTTGGTACACCCGCTAGTTGCGTGAT |
| | | CGATATTGATAAAATCACGAAAAAAACCCCAGTTAATAATCCTTTACTTTACCTGAGTCTGCATCTCATCCCTAT |
| | | TGAATTCGTCAATCAGCTCTCTCTGCAGATAAAGAAATATACTGTACTCACCGGTTCGGAGAAGTTCGGAAACA |
| | | TCCATAATGATATCAACCTGTATCTGACAATGCACAATGCCATTTTGGATATAAAGTGGCTCGACAATCGTGATG |
| | | AATTAGCCAAGTTTTATGACAATAAAGCGCTGTTTGAACAGGTGTTTAGTCAGGATAATAAGCATTTGATTCCCG |
| | | TCAAAGGTTTGCGCGAAATGTTACGATTTGGCAACCTTAGCGAACTGGGAGTTATATTTGAACGTTCAATCGTTG |
| | | AAGCCAATGAGATTAATTCGCTGCAAGACAAACAGAAAAATATTGCTGCCATTCAGGCTAAGAAAGCAAAAGC |
| | | CCATCAGAAACTGTCACAGCAAAAAGACATTAACAAGCAAGACAAAGCGGATTACAAGAGTGACGTAGATAAA |
| | | GTGTCCGAGTACAACCACCTGAAAAACCAGGTTTATCTTCATAATCACAAACAGACCCATCAGTTGATCATGAC |
| | | GATTAGCGGCCGCTTAGTGGGGTTTTCTCGCCGGTACGAGCGAGATATCTATTTTGTCCTGTTGGCTCTGCTGAC |
| | | ACTGAACAAAGTGAAAACGGACGAACTGTCACAGAATTTCCTGGCGTCTATTCGGTCGGGTCAGATAATTAAGG |
| | | CATCCAATCAACTGGGGGAGCAATACGCAAACATCAAAAGTAAGCTGAACCTGCTTTTTCAGATAGAGAAGCAT |
| | | AGAGAAATCCGGAATCGCTTTGCACATTTTGATGATCTTCGTAAAGCGGAGTCGTTAAACATAACGCAACAGAT |
| | | TAATGAAGCACGGCAGCTCATGAGCTATGATCGTAAGAGTAAAAATTCCGTAGTGAAATCAATACTGAACATTG |
| | | TTAAGAAGGACAACATCTCAATAGATTTTACTATGGACAATCAGCATAATCTTACCTTGAAATCTATCAAAGCG |
| | | GACTCCATTATTCATCTGAAAGATAAAGATATTTCTGAAAAGCCCCAAGGTAAAAAGTACAAGAATGCTGTAAA |
| | | AAAATTATTCGAGAATACTGTGTAA |
| 344 | JW13169 | ATGAATGTTATTGAGAGTTTCTTACCGCTGAACACCAAGGAAAAAATCGATCAGCTTATGGCTATTATTAAAAG |
| | | TGAACCTAAAGCAATGAATCAAAATGCCTGGCACATGATTTTTGGGAACATCGGTAAGAGTAAAATGCAAATGT |
| | | ACTTTATTGAGAAATACCCGCAGTCACTGTTTGCTGCCCCTGGAAATTATATTCCGTCTTGTAATGGTCAGGGTC |
| | | AGGAAGAAAAAAATCCTCAGCAGCCTCAAGTCCAGCAGCACGAACGTCAGACAGATATCCCAGTTCCGCCAAC |
| | | CTCGTTGAAAGACAATCGCTTTTTGTATGCGACGTACATTGAGATGGCGTTTCACAATATGTTTCTGAACATCAA |
| | | GCACATCTATGGTGTAGTGTTCGGAAGAGATATAATGGCTGAAGCCAAAGCCAATTATGAGGCACTTAACCCGG |
| | | AAAAAAAATGGGATGAGGATTTTGCAAATGAATTTCTGGTATGGAAACCAATGTTCGAGGCTTTTAACAATGGA |
| | | AATGTCGAGGAGAAACAGAAAGTGGGTGAAATGTTATCACGTCACTTTCCGCTGTTAGTCCCGTTTACTGATTTT |
| | | ACGAACCATGAGTCCAACTATAAGAATCTCACTATTGTCGGTATTTTACGGCGCCTGTCGCAGGTCCTGCGTGTA |
| | | TTGCGCAATCTTTACTCTCATTATAGAATAGAACTGTTCGAGAACCAGAAAAAAGTATATCTTGACAATGAATAT |
| | | CTTATCATTCGCTGCACCATGAATGCCTATATGGGGGCTCGGCGCGTTATTAAAGATCGTTTTTCATACGATGAA |
| | | AAAGATATGCGTTGCACTGATCAATATCAATTCGTAGATGAACGCGGTCGTCGATTGAAGGAAAAAGTCGAAAT |
| | | CAAAGGATTTCGTTATAAGATTGGGGAAAAAGGAAAAGATTCTAAACTCCATTTCACGCCCTTTGGCTTGGTGG |
| | | CTTTCATATCGCTTTTCTTAGAGAAGAAGTATAGCAAGATTCTGACTGACAAACTTAGACTGATTCCCATACAGG |
| | | ACCAACATATTATTAACGAGATGTTGGCGGTCTATCGGATTCGTTTAAACGCACAGAAGCTGAACATATCTAAA |
| | | GATGCAGACCTTCTCGCCTTAGATATTATTAATGAGCTGCAAAGATGCCCGAAAGATCTGTTTAGCTTACTGAGC |
| | | CCCAGCGATCAGAAAAAATTTCGTCATGAATCGGAAGCCAACGATGAAGTTCTGATGGTTCGTCATTCCGATCG |
| | | ATTTCCATTTCTGGTTATGAAGTATATTGACGACTGTCAGCTGTTTGATAATATACGATTCCAAGTATCTCTCGGG |
| | | AAGTATTTTTACAAGTTTTATGATAAAAATTGTATCGACAGTGAGACCAGAGTTAGAGCACTGAGCAAAAATCT |
| | | TAATGGTTTTGGAAGACTCTCGAAAATAGAAGCAATGCGTGAAGGTTGTTGGGAAGATTCTATCCGCCAATATG |
| | | ATGATATACACAAGAACACCGTGGATGAGAAACCCTATGTCACAGATCATCACGCAAAGTATGTAATCAATGGT |
| | | AATCGGATTGCCATGCGTATAATCCGTGAAGAAGAGAAAGCTTATCTTCCTGAATTAAACGCAGAAGGCGTTCG |
| | | CAATCTTGCACCGACATGCTGGTTAAGCATCTATGACCTGAGCTCTCTGGCATTCTTGTTATACCTTACCAATGG |
| | | TGACTTTGTGGAAGGAATAATCGAGAATACCGTAGAGAATTACCATAAACTCTTCAAAGATATTGAAAATGGCA |
| | | CGATGCTGCCGGTGGCGAATGAAGAGGAGCTGGACAGCCTGTTGTGTCGCAACTATGGGGGAATTAAACAGAA |
| | | AGACATCCCAAAAAATATGTTAGACTACCTGCTGCGGAAAACATCCGATGCTCGCAAGCGTTTCGTGGAATTTT |
| | | CGAAAAGTATCATCGACCGGATGATATCACAGACAGAATATAAGATAGAACAGATCAAACGTCAGGAAGAACA |
| | | ATGTCGCCAGAACAAAATTGGCAAGAAATCCTATGTAATCATTAAAGATGGTAAACTGGCGTCCTTTCTGGCTA |
| | | ATGATATAATGCTGTTCCAGCCAAACGATAGCGAAAACTCCAATAAGCTTACCGGCTTAAACTTTCGCATTTTAC |
| | | AAAGTACACTGGCCACAATGAAAGAATGCGGCATTGATCAATTAAAACGTACGTTAACGTCGGCTCATATTATT |
| | | GGCGAAGCGAATGACGAGTACTGCAATCCTATCGTTATGAAAATGATGCGGAAACGTGTGCCAGAAAATACAC |
| | | GTGACTTTTACAAAAGTTATCTTAAAGCCCGGCTGGAGTATCTTAATGAATGCAAACGCAAAGCGGATTACGAA |
| | | AATCTGTCCTTTCTGCATAGTAACCGTTCAAAGTGGCAAGAACATGATGATGAATTTTGTCGTTTGTTGGCGGGC |
| | | CGCTACCTGCACGATGAAAATGGCGGCACGGAATTTGAAAAATCAATTAACTTGCCCTCTGGTATGTTTGATAA |
| | | GTATATCCGTGAAAAACTGTGCGAAGTTCCGGCGCTGAAAGACATTGCGAACGATGAGACCAAAAACGTTTCAT |
| | | ATTTGATCTACGCATATTTTAAGAACGTTTGTAATGACGATTGCCTGTTCTTTTACGAAGCTGGGCGCTCATACC |
| | | GCTTATTCAAACTCTTAGGCATGAATGGTTCAAAAACTGCGCAGGATATTCGACTTATGCTGAACAGAAAGCAC |
| | | AAAACTTCACTGTACAAGGAAATAGATAAGTATCTCATCAGCATTCCTCTGGCGGAGCGTGAACAAGAGAAAGC |
| | | CCGCTTAATGGGCTTATTAAAAGGGATGAAAAACAGTGAAACAGACTTTAAACGCTTCAAGATACAGGACATTC |
| | | TGCTGTTCCTCATAGCCAAGAAGCTGTTCAAAGACAATGTGCAGGACGATGTTCAGCATACTGCCTTCGAGAGC |
| | | CTCAAACTGAAAAGTCTCAAAGATACCGACGGATTATCTCAGAAGATTCGAATCTCTGTGAAAATTAAGAGCAA |
| | | AAATGGTTATGAAAAGGAGATCAAAAAACCGGAGTTGAAGCTCAAAAATTACGCGGATTTCTATAAACTTCTGA |
| | | ACGACCGGCGTTTACCTGCATTGTTGGATCTTGTCAGAGAACATTCAATCGATTATGATTCTCTGGTGGACGAAT |
| | | TCGGTGGGTACGATAAAGTTCGACCGAATGTTATGAAAGATGTGCTGAAATACGAACGGAAATATTATGATAAC |
| | | CATGCTGACTCCGATGAGTCATTAGACTTCGGCAGCATGTTAGATAACTCCAGTTTTAACGAGGAAGAGAAAGA |
| | | TTTGTTACGAAAAATACGGAATAGTTTTGCCCATAATACCTATCCTAAGTATTATATTGTGAACAAAGCGGGCGA |
| | | TCCACATCTGCCCGAAAAAGCAATCAAATGTTCCGAAAAGTTTTCTAGCGACATTAATAACGAGTAA |
| 345 | JW13170 | ATGGCCTTCGATAAACCACGTCCGAAACGGCGGCAGGCACGCTCCGATTTCTACATAACCGATAAGGCGATAAT |
| | | GGGTGGATATTTTAACCTTGCGCAACTGAACTTTTTCAAGACGTTAATGGAGATCTTCACCAAGGCCGGTATCGA |
| | | TGTTAGTAAGATCAAGCAAGACAATAGTCCAAAGTATCTGATGATCTTGATCAAAAAACTGACCCACGATGATG |
| | | AAAAGATTGATGACAAGAAATGGGCGGACGCCTTAGACCTTAGCAACGAATGTCTGCTCAAACTGCAGCAGTTA |
| | | TTATACAAACACTTCCCTTTCTTAGGCCCAGTCATGGGTGGGGAAGCTAGCTATAACATCTACAAACTCAAAGAT |
| | | GGCCATCCCGAAGTGAAGGTGGCAAATGATGTCATGCGTGGTGTGAAACTTGAGGATTGTTTAGCAGTGGTGAG |
| | | ACATTTTGCATTATGTCTGAACGACTGTCGAAACTTCTACACTCATTTTAACCCGTATAACCCGATTGAAGCGCA |
| | | GAAAGAACAGTATCTGTCCCAGAACAAAGTGGCAGTTTGGCTGGATAAAGTGAAAGATGCTTCCCGTCGCATCA |
| | | TCAAACAAAATAATCAGTTAACGTCTCAGGACATGGAATTTCTGACTGGGATAGACCGTATGAAACCCCAGGAC |
| | | AAGGTTGATGATTTTGGAAATATCATGACCGATCGTCGTGGCCGTCCCATCAAAGAATACGTTGAGTACGAAGA |
| | | CTATTATTTTCGAATTCGCGGCAAAAGATATCTGGTGGATGCGGCCGGCGCAAAACTTGTAGACCAGGAACCTC |
| | | GTAATGCACTGTCTGATTTTGGCGTAGTATTCTTGTGCACGTTATTCTTAGAGTCTGACCAGGCTAGACGAATGT |
| | | TAGATGAGTTACGCCTGTTCGAGACCGGGCCGTACGACGGTAGCCGTGATGGAGATGAATTCAAGAATGACATA |
| | | TTACGGGAGATTCTGCTTTTTTACCGTACGCGTGTACCGAGAGGTAAACGCTTGGATCCTATGGACGATACGACA |
| | | CTCCTGGCAATGGACATGCTCAACGAATTGCGGAAGTGCCCGATGCCTTTATATGATGTTTTAAGTCGGGAAGG |
| | | TCAGGGTTTTTTTGAGGACGAGGTTAAACGCCCTAATGATCTTACCCCCGAAGTTGCCAAAAGATTGCGTAGCTC |
| | | TGATCGCTTCCCGTATCTTGCACTCCGCTACATCGACCTTAATAAGTGTTTTGATAACATCCGTTTTCAGGTCCAG |
| | | CTCGGCAAGTTTCGTTATAAGTTTTATGATAAAACCACTATTGATGGGGAACAGGTGGTTCGCGGCTTGCAGAA |
| | | AGAAGTGAATGCGTACGGTCGCCTGCAGGACGTTGAACATTACCGTCAGGAAAAATATGCTGACATGCTGCAAC |
| | | AGACTGAACTGGTGGAAACAGGAGAGGAAGATATCACAATTGCGAATTTTATTCCGGATACGCCGCAGTCATCC |
| | | AGTTACCTGAGTGATCGTACCGCGAGCTATAACATTCATAACAACAGAATTGGTTTGTTTTGGAATATGCCGGGT |
| | | GAACAGGAAGTTCTCGCGGGGGACGAAAAAATGTATCTTCCTGACCTCAATGTAGATGATAATGGTAAAGCGG |
| | | ATGTATTTCTTCCAGCACCAAAAGCATCTCTGTCCGTACGAGATCTGCCAGCTCTGGTTTTTTATCTTTATCTTCA |
| | | AAATCAACATCCCGATTTGACTCCTGCGGAAGCTATCATTCAGCAGAAATATAATGCCTTAGTGTCGTTCTTCGA |
| | | GGACGTCTCAACCGGCCGGTTATTGCCAGTTAAAGATATAGCTGAGCTCGAGGCTGCCATTGACAAGTATGGCC |
| | | ATCTGACCGTTCATGAAATTCCGGAAAAGTTGCGCGACTATCTTACCGGAGTTGCCGGTACAGAAGATGATGAT |
| | | GATTGTGCAGACCGGCTGGACTGCTATGCAATGGGTATTCTGGAAAAACGCTATCGCCGTGTCGCCGCCCATAT |
| | | TGATCAGCTGAAAGAGGCGCGCAAAATGGTCGGTGATAAGATGAATCGTTATGGAAAAAAATCATATATTGAC |
| | | GTGCGTCCGCGTCGCCTGGCAGAACAGTTAGCCCAATCAATCGTTGATTGGCAGCCGGCCACTGACAACGGCCG |
| | | TCATAAAATGACTGGCAAAAATTTTCAGCGCCTTATTGCCTTTCTGGCGAGATATACTGATGAAGTTCCGGTTCA |
| | | AGAAATTAAAGATCTGATGACAAGAGCACGCCTGCTGGATGGGCCTGTGGCGCACCCGTTCCTTCAGCAGGTTT |
| | | TAGATAAAAACCCACTGAACATTGAGGTACTTTACGACTTGTATTTTAAAGAGGAGAAGTTGCACCTGGAAAAT |
| | | TACCTGGAGATAGAGACCAACAAACAAGGAGAAGAAGTGTTTCGTAAAAAAGCTAGTGTTGATCTTTCACGCAT |
| | | TCCTTTTCTGAACAGCGGTCGTCTGCGTTTTCATAAACGAGATTCTGCCAGTTTTCAGAGACTCGCAGCTAGATA |
| | | TATGCAGGTAGACGGACGACAGTCTACTATATTATTACCCGATGGGTTGTTCACACCACATATACTGTCAGTCCT |
| | | GAAATCGGAATACCGCGACCTTCAGGATCATTTGCAGGATCAAGAGCTCAACCGTAATGCTTCGTATCTTATGTC |
| | | TGTGTATCTTGAATGCGTCTTGCACGATTCGGCCCAGCCATTTTATCTGAGCAATAGCAATGATTCTCCGTTCGC |
| | | CCGTCGATACGATCTGTTTGCAATTTTGCAGAATAAAAAAAAGGGGGCGGAGTTACTCCCCAACCCATTAGCTC |
| | | CGGTAGAAATTAATCGGCGGCTGACTGCAAAAACACCAGATGGCTCTGCTAAACTTATTGAACGTGATATCAAA |
| | | GCGCTGTTAGACGATATGGCGCGCAAAACAGAGGAACGGATCCATGATCTGCAACAAGAATATAGCCGTAAAA |
| | | AAGAAAAGCAAGCGCAAGAAGAACGGGTGAAACTGGAGCGCTGCTTTATGCGCAAAATTGCTGATGTTAAGAA |
| | | ACAGGCCCAAGAGGAACGCATTAACCTGCAGAGACGACTCAAACACAAAATTACTGATGTCAAAAAAAATGAA |
| | | CGGACCATCCGTCGTTACAAGACACAAGATATGGCAGTTTTTTTAATGGCGAAACAGATGCTGGCCAACCAGCT |
| | | GTCCTGGCAGAATCAAGAGGGTGAGGTCCCGTTTAAACTTGCTAATGTGTGTAATGAAGGCTTTTTACGGCAGA |
| | | CGATGAAAGTTAGCTTTCCTTATGAGGTCCCTCTCAAAGAGGACAAGACCAAAAAGGTAACGATAGATATCGTT |
| | | CAAGACAATATGAGTCTCAAAAATTATGGGCTGCTGTACAAGTTATATCACGATGAAAGAGTAGTGACGCTGCT |
| | | GCAAAATATCGTGGATCAACAGCAGATATCATTTGCTGAATTAACAGCAGAACTGTCCTTATATGACCAAAATC |
| | | GGTCAAAGGCTTTCAGTAATTTTCAACAGTTCGAAAAATTAGCGTTCGAAACAAATAAAGAGGCCCTGACCCAT |
| | | CCGGAACACCGGGGATTCTTTCTGGAGAAAGTGAACAAAAAGAATCAGGTCATACGTTACGAGCGCCGTAATA |
| | | ATTTTGACGCTTTGCTGGATCTGTTGGATGCAATTGAAGCCCATCAATTAGACCCTCGTGAACGCCAGTGGCTGA |
| | | CGAGCGTCCGTAACGCATTTTGCCACAATTCGTATTACAAGATAGATATGCAACAAATAGAGAAGAAATTGCCC |
| | | ACGATTATTCAACAGATTGTAGAAATAGTACAGCGAATTACAGATAAAGCCTCGCACCCTTCAAACAGATAA |
| 346 | JW13171 | ATGTCCACGAGTGACTCTTTTTACTCGTTTGGAGTAGATTATACCAAGACCTATGACCAACAGCCGGATTGTCGC |
| | | CGCCACGTATTTGGCGCGTATGCCAACCTTGCGAGACATAATATGACCCTGGTTGTAAACACCATTATGCAAGCT |
| | | ATCGGCATGCCAACCTTTAATGAGAACATTATCTCAGACGCTTTCAATAGCACACACAGAAAAGAGATAGATAA |
| | | ACTGGATAACATTAAAAAAGTCCTCTTGCAGGAGCGCTTGTATCGTCATTTTGCTTTTCTGAAACGGATGAAATT |
| | | AGAAGATAGCAAGAAAAAGACTGTGCAGCTGAACACGTTGTTAGAGACTCTGAGCGATTTTACACGAATGATG |
| | | GCCTATATGCGTAATTTTTACACGCATTATAATCCGTATAATTCGCGGGATGAAAAAGAAAAGCAAGGCGATCT |
| | | CAATAAAAAAATGGGCGAACGGTTACAATTCCTCTTGGAGAATAGCCTTCAGATGTTGAAAGACACAGAAAAG |
| | | TTAGATCACGCCAAAAATGAAGTCTTGTCCTCTCTGCGTGTTCCAGAACGGAAAACTATAAGACCGGGGGATAC |
| | | AGAATACTCAAACATTCACGACCTGTACTTCACGAAAAATAAGCGAAAAGGGATCAAGTACGACAAGGACACG |
| | | AAAGTGTTCACGCAGCAGGTCTATATCCTTAACCCCGCTTATCCAGCCAAAATGACCGATAGTGACGGCTATCT |
| | | GTCTCTTGCTGGTATGGTGCATTTTCTGTGCCTGTTCTTAGAAAAGAAGATCGCGTTCGAACTGATGGACGAAGT |
| | | TGGGTTTACACAGCAATGTCATTTTAACGGTAGAGATGCAGAGCAACAATTGTTATACATTAAAGAGGTGATGT |
| | | GCATGAATCGTGTACGTATGGCGCAGCCTCGTTTAGACTCTGAAATGAGTGACACCGCCTTAGCACTGGATATG |
| | | ATTAATGAATTACGCAAGTGCCCAGAACCTCTGTTCAAAGTGCTGGATAAAGAAGCTAGAAATGAATTCAAAGA |
| | | TGATGCAACTGTTCAGTGGGAAATGGAGCACAATGTTGAAGCGCGTCCAGGGGAAGATGAAGAGAATAACAAT |
| | | AATGAGGATGACATCGAAGAACCCGACGAAACAATTCGTGAGGATAAGGCTGTTCCGCGATCTACGTTTGTACG |
| | | CTGGAATGATCGTTTTCCGCAGATGACGCTGTCCTACATCGATATGCAGAGTGTGCTGCCTACCATACGTTTTCA |
| | | ACTGTACCTGGGTAAGTACAGATTTGCATTTTATGAGCATGATAAAGAATATTCCGTTGATAATGCGGAGCGCTT |
| | | ACGTGTGTTACAAAAAGAGATGCATGGATTTGGACGTCCTCAAGAAGTCAAAGCGAATCTTAAAGATAAGTGG |
| | | AAGGACTACTTTGAACACAAAACAATAGAAAATGGTCTGACAATGAAAGAACCCGATAAAGCAGGTCAGGCCC |
| | | CGTATGTCACGGAACAGAATCCCCAGTACGCAATTGATGAAAAATCTCATTCGATAGGTATTCGTTGGGGTGAG |
| | | TGGAGCTCCGGTCATGGTGGCTCCCATTATGGTGACCTTGACAAACTGAACATGTTCATTCCTTGTCTGCCTAAA |
| | | CCGAAAGCCGATGAAGATAAAGATAAACAGCGTAACCATGCCGAAAAATTATTACCGCCTAAGTGCATGCTGTC |
| | | CCTGTATGAGCTGTCAGGTTTAGTCTTCTATCACTACCTGTTAAAAAAGTGCGGAAAAGAAACCGGACAGGTCG |
| | | AACGTCATATAATTACCTACCACGATAATCTTGTCCGATTTTTGACCGATGTTAAAGAAGGTACTATCATTCCGG |
| | | TTACTGATGTGGATGACCCTAAAGGTTATCTTGAAAAAATGTTGATGGAAAGTTATGCCTTGCGTTTATCTGACA |
| | | TTCCAGAGCGTATAAAGAAGTACCTTCTTAATGAGACGAAAAGTCGCGAATATCGGTTGAAAGAAAGTGCCAA |
| | | AAAGCGGCTGGAGATACGCCTCAAAAAAGTGATGAAAGATCTTGAGAGCCATCAGGCCAAAAAAAAGCGCATT |
| | | GGAACAAAAGAGAACAAGTTTAATCGCAAACGATCAACCATCAAGACAGGGAAACTTGCGAAAGATCTCATTC |
| | | GTGATATAATGGATTGGATGCCTCGCGGCAGTGTAGGGCGTATGAATCTTAGCGGTGAATCTTATATGGCAATC |
| | | CAGGCCGCGTTGACCATGTTAGGACAACAGTTTGATCGTGGCAAGGCTAATGTGGAAATCTATGATCTCGTCGA |
| | | TATTTTGGTGAAAGCCAAACTGATAATGCCAGATAGCGATGTTGAGGGCGCAGATTTTAACCCATCACATTATC |
| | | ATCCGTTTATGGATGATGTTCTGACAGAAGATCGCTGTGGCCCGGAAACTTCCTTAGAAGATGTCTATGAATGGT |
| | | ATCTGGAAGAAGAGATTGCGCATATTAAGCGACTGATTAGCTCAATCAACAAAGTGTCAACAGATAGCCTGCTT |
| | | ATGAAGCAGTATAATCTCATCCCCTTCCTGCACTGTGGACGCGAAAGATGGAATAAGGTTGATGACGAAGCTAT |
| | | TAAAAAGTTGGCTGAGAAATACCTTAACAATCCAATTCAGCTTCCAAACTCGCTGTTTGCGAAACCCATTTTCGA |
| | | ATTACTGAAGGAAGTAGCCCAGGAAAAAAACTATGATCGGTTATCACAAGCTCTCGAGGCAGCTAGCCAGGAT |
| | | GCGAATAACCTCTCAAAAAACACCGCGTATCTCATAGGGTTATATTTTGAGATGGTTGAAGGCGATCACAGCCA |
| | | AGCATTTTACAATTCATGTCCAGCGGACGATGGCACCGTTAGTCCGTATCGTCACGTATATAGAGTGTTCAAAAA |
| | | ACTCTACGGCGAGATCATCGAAAACACTAACCGCACGACATCACCGGCATATTCTAAGAAAGAGATCTCCGATA |
| | | TCTTAGCAGGGCGTGAAGTGAATGGCAAAATTCTGTCGGACCGTATTTCTCGCTATGTCCAGAAAGCCGTCGAT |
| | | GAATTCAAAAAACAGAAGGACGATGACCTTAAACAATATCGTATCGAAATAAAGGATGCCGCCTTCAATGAAA |
| | | ACCGGGTTAAACATCTGAGAAAGAAAGCATACGAAATATTTGAGGAAGTTGATGTTAAAGTTGACCAAAAACG |
| | | GGAAGAATTTGAGCGGTTAACCGATCAGTATCGTCAAGAAGTGGAAGCTAAACTGAAAAAACTGTTGAAAAAG |
| | | GTTCAGCTTAATGAGCGCGCAATTTTACGGTTCAAAACTCAGGACATTCTGTTACTGATGATGTCTCGTGACATT |
| | | CTGAAAATGGGGAGTGATAACGAGTTTAAACTGGAAAATGTTTTGTCGGAGTCCTTACTTGAGAAGCGAGTTGA |
| | | CTTTTCGTGGAAAGTAAGCGTGAACGACCAGACTAAAGATAATGTGACCACTTATAAGACGATCGAACAGAAA |
| | | AACATGAAGATGAAAGACTATGGTCAATTTTACAAATTTGCGTCAGATCATCAAAGACTGGAATCTCTTCTGTCC |
| | | CGTTTATCTCCGTCAGTATTCCAGCGCGCAGAAATTGAGAATGAATTCAGTTATTATGACTCAAATAGAAGCGA |
| | | AGTATTCAGACTGGTCTACATCCTGGAGAGTTTTGCTCTGGAGATGAAGCCGGGTCTGAAAGACGACAACAATG |
| | | CGAAAGAACCTTGGTTCAACTACATTCGAAAGGGAAAACCTTCGCCGAAACGGAACTCTTTTATCGAGCTGCTG |
| | | GAAGTGCTCATAGCAGGTGCAGATGGCATTTTAGACAACCAGGAGAAAGAACTGCTCCGCGAGATTCGCAATG |
| | | CGTTTGGACATAATACCTATGACGTGGATTTCGAGACTATCTTTAGCGGGCCGCGCAAAAGTAAACGCAAAGTA |
| | | CCCGAGGTAGCAAATGGTATTAAAGACAATATGGAAGAAAAAACAACGAACGTTTTGGAAAAAACTAAAAAAT |
| | | AA |
| 347 | JW13172 | ATGAAACAGAATGATCGCGAAAAGAATAGCAAAATTAAAAAATCTGCAGCCAAAGCTGTTGGGGTTAAAAGTC |
| | | TGGCACGCTTATCAGATGGCTCGACAGTAGTTTCTTCTTTTGGAAAAGGTGCCGCAGCGGAGTTAGAATCTTTGA |
| | | TAACCGGTGGGGAGATTCGTAAACTTTCCGACAAAGCGATCTTAGAAATTACTGATGACACACAGAACAAAAAT |
| | | GTGTATAACGTGAAGAGTTCCCGAATTCCGAATCTTACGGCGCGCACCGATAAACTGAGTGATAAAAGTGGAAT |
| | | GGATGACCTTGGTTTTAAACGTGAACTTGAACTCGAAGTCTTTGGTCAATCATTTGATGATTCGATTCATATACA |
| | | AATTGCCCATGCCGTTTTTGACATCCAAAAAAGCTTAGCAGCGGTGATTCCAAACGTGCTGTATACCCTCAACAA |
| | | TCTTGATCGGTCATATTCTACGGACGATACGAATGGGAAAAAGGATATCATTGGCAATACACTTAACTATCAGC |
| | | ACTCCTACGATAATTTCGATAAGGAAAAACTGGGTGAGTTCAAAGAATATTATAATGCCGCAAAGGATCGCTTT |
| | | AGTTATTTTCCAGATATATTATGTGTTCTGGAGAAAGTTAATGGCAAAGATCGTTATCAGCCCAAAAGTGAAGA |
| | | AGATGCCTTTAACGTCTTATCCTCTGTTAATATGCTGAGAAACTCATTATTTCATTTTGCACAGAAATCAAACGA |
| | | TGGTAAAGCTCGTATAGCTGTCTTTAAAAATCAGTTTGATAGCGGCTTTAGCCATATTACCAGCACAGTTAATAA |
| | | AATCTATTCGGCTAAAATAGCCGGGGTCAACGAGAACTTCTTAAACAATGAAGGAAACAATCTGTATATCATAC |
| | | TGAATGCCACCAATTGGGACATTAAAAAAATTGTTCCGCAGCTTTATCGTTTCTCCGTGTTAAAGTCGGATAAGA |
| | | ATATGGGTTTTAATATGCGTAAACTTCGGGAATTTGCAGTAGAGTCGAAAAGTATAGACCTGTCCCGCCTGAAC |
| | | GACAAGTTTCTCACCAATAACCGTAAGAAGCTCTACAAAGTCATCGACTTCATCATCTACTACCACCTGAATAA |
| | | AGTGCTGAAAGACTCGTTTGCAGATGGCTTTGTGGCAGCCCTGCGTGCGAGCCAGTCCGAAGAAGAAAAAGAA |
| | | AAACTGTACGCACAGTATTCTGAGCGTCTCTTTGCAGATGAAGGCTTAAAATCTGCCGTAAAAAAAGCTGTGGA |
| | | CATGATGTCAGACACTAAGTTTAGCATTTTTAAAAAGAAGGCCCCACTTGATAAGGCCTTGATTGAGAACATTA |
| | | AGGTGAACTCAGATGCAAGCGACTTCTGCAAACTGATCTACGTTTTCACGCGATTCCTGGACGGAAAAGAGATT |
| | | AATATACTGTTGAACAGCTTAATCAAAAAGTTCCAGGACATACACTCCTTTAATACCACCGTTAAGAAACTCTCC |
| | | GAAAACAATCTGATAATTAACGCGGATTACGTAGATGATTATAGCCTGTTCGAACAAAGTGGTACTGTCGCTAG |
| | | AGAGTTGATGCTGATCAAGAGTATTAGTAAGATGGATTTCGGACTGGACAATATTAACCTGTCTTTTATGTACGA |
| | | CGACGCTTTGCGCACCCTGGGTGTATCAGATGAAAACCTGCCCGAAGTGAAAAGAGAGTACTTTGGTAAGACTA |
| | | AAAACCTTAGCGCATACATCAGAAATAATGTATTGGAGAATCGGCGTTTTAAGTATGTAATTAAGTACATTCAT |
| | | CCGTCTGATGTCCAGAAGATCGCCTGTAATAAAGCTATTGCGGGTTTCGTTCTGAACCGTATGCCTGATACTCAG |
| | | ATAAAACGCTATTATGATTCGTTGATCAATAAAGGGGCGACCGATATGCAAGCTCAGGCGAAAGCGTTATTAGA |
| | | TTGCATTACAGGAATTTCTTTCGACGCCATTAAAGATGATAAACATCTTCATAAGAGTAAAGAAAAAAGTCCTC |
| | | AACGTTCAGCCGACCGCGAGCGGAAAAAGGCAATGCTGACACTGTATTACACAATCGTTTATATCTTCGTGAAA |
| | | CAGATGTTGCATATCAATTCACTCTACACAATTGGCTTTTTCTATCTTGAACGGGATCAACGCTTTATCTATTCTC |
| | | GTGCAAAAAAAGAAAACAAAAATCCCTCGAAAAATTCATACTTGAACGACTTTCGTAGCGTCACGGCGTATTTT |
| | | ATTCCGTCAGAGATTATGGAGCGCATTGAAAAAAATGAAAACAAAGGATTCCTGAAAGAGTTTGAGGAAGCGT |
| | | GGAAAACTTGTGGGGAAACAAGCCGGTTAAGAAAAGAAGATGTTCTGTTGTATGCTCGCTATATCAGCCCAGAC |
| | | CACGCTCTCAAAAATTATAAGACGATCCTGAACAGTTATCGGAATAAGATAGCACACATTAATGTCATAATGTC |
| | | CGCGGGCAAGTATACTGGCAGTATTAAACGAATGGATTCTTATTTCGCTGTGTTTCAGCATCTTGTACAATGTGA |
| | | TATTTTGTCAAATCCTAATAATGCTAACTGCTTCAAAAGCGAATCCCTCAAACCTTTATTACTGGATATGCGATT |
| | | CGACGGCACTGATGAAAAATTATATTCTAAACGTCTGACCAGAGCGTTAAACATACCGTTTGGTTACAATGTGC |
| | | CGCGCTACAAAAACCTGACGTTTGAAAAAATCTATTTGAAGTCGTCAATTAATGAGTAA |
| 348 | JW13173 | ATGGGCAAAAAAAAGCATGATAAACAGGAACCTATTGCGAATCGTCACATTTTAGGCACCTACTTCAATATGGC |
| | | GTTTGAGAACTTCAATAGAACAATGTACATTCTCTGTGAAAAAAAGCTGAAAATTAGCACCTCAGAATATGGCA |
| | | CTCCTGATACTGAACACCTTATCGCAGATCAGCCTATTCAGCTGCGTAAAGCTCTGTTCCGTTATCTTCCGTTTTT |
| | | GGCACCTATTATGGATGATTGGGTAGGATATCAACTCAGAAGTAAAAAATCCAAAGGTAATGGCGGCGACGAG |
| | | AACATTACCCAAAAACAGAAAACAGCCAAACTGGACAGTTCAATTGAAGATATCGTTAGCCAGTTAAACATTTT |
| | | TGCCCAAGAGTTGCGTTACTGTCGAAATAAGTATACCCACAAAAACGCCTATACGTTAGCCTGGGAAGATGAGG |
| | | AGCACCTTCAGAGAGAAAAGCAGATAGCGAAAATGCTTGAAGTGTTAATGAAGGCAGAGCGTGAACTTTTGAA |
| | | AAATCGTAAAGATCGCGAAAAGAAATCGTCGGCGATGGAACTGTTTTTAAAAGACGGGGACCAGTATTACTACC |
| | | GCACAAATAAGGGTAAAACTGTGCGCAACAAACAGTTCTATTTCAATCCCGCCTCATTCGAGCTCACGAAAAAG |
| | | GGGCAGGTTGATTACGACACTATCGCACTGACCGACTTCGGGCGCTTTTATTTTTGCAGCCTGTTTCTTCGGTCA |
| | | CGCGATGCGGAGCGGTTTGCAACGGAAATGAAAATTTTTATTGAGTCTCCATGGCCGGATATTAAAGATAAGGA |
| | | TTCAAATCCAACCACTTATACTCCTAGCCCGGAAAATAAAATTATGCGGGAAGTGATGCAAATGCATCGAATCC |
| | | ATGTACCCCGGGAAAAACGCATCGATGCCGTGCCATCTAAAGGTACTTTGTTAATGGATATACTGGAAGAACTG |
| | | CGGCGCTGTCCGAAAATTTTATTTGATACGTTTTGTCCGACCACGAAAAGCAGTTTCAATCACATAGTAATTCAG |
| | | GATGATGGTACCAAAACATCTACATTAATGGTTAGACATCAGGACCGCTTTGCTCAGTTAGCATTGCGGTATATT |
| | | GATCAAAACGGTTTATTCCCGAATATACGTTTTCAGCTGCGACTGGGCTTATTTCGTTTTAGATTTTATGATAAG |
| | | ACCCTGATTGATGGTACCCCGTGCATCCGTACCGTCCACAAAGAGCTGAACGGTTATGGTCGTCTTCAAGTCGTT |
| | | GAAGAAAAGCGCGTAACTAAGTGGGGAGACCGTTTTCAAAAAGTAACCACAAAAGAAAACGAAGGATATGAG |
| | | ATTGAACAGCTGCAGCCGGATACACCACAAACGGATCCTTATGTTACAGATTGGCGTTCTACGTACAATATTCAT |
| | | ACTGGGCGTATCGGCATGTCTTGGGGCGAAGTGAATGTCGGTGGGAAAACCGAAGCCGGTAGCGCCCTGCTGCC |
| | | GAAAGTTGATCGCAAAAGTAATGGAGACGATTGCTACTATATTCCCGATCTTCCGACTTTGAAGTCGGATGACG |
| | | GGAAAAATAAAGATCGTAAGGTCAACATCCCACAGGACGCGCCACTGTGTACCATGAGCACCTATGACCTTCCG |
| | | GCACTGCTTTTTTATCAATATCTTATCGACAGTTATGGCAGAAAAGGAGCAGATGAGAAAAAACTCCCCACATC |
| | | CGAGAATATAATCCTGGAAAAATGCTATGCCCTCAAAGCGCTGTTCAAAGCCGCAGAAGACAATACCTGTACTC |
| | | CTAAAAGTCTCCGGGAAAAAGAACGGGAATTGGGTCTGAGCGACAACGAGATTCCGAAGAAACTGCTGTCATA |
| | | TCTTGATGGCGAGAAACACAACCCACAGCAAAATCGTAAAAAACATGTAGAGCATGTTTTAAACGGCATAATTA |
| | | ATGACATCAATGGGCGTTTAGAGAGTTTTGAAAACAAAGAGAAGCGAATCAAGGCGGGCGGGCGTAATAATCG |
| | | CTATGGTAATCCACATCATGCCGACGTCCGCCATGGATCCTTAGCGCGCTATCTTATGCGGAGCATGTTACGTTG |
| | | GCAAAGTGTTGACGTTGAATCACCTCATTTTGGTAAGCTGACGTCATTGAACGCACGTACCCTGATGGGGGCTTT |
| | | AGCAGCTTTTGGTAGTGGGGATGAACGCTTACCGTTGAACGAAATTCTCACAGCGGCGAATATGATTGGTGGTA |
| | | ATAATCCACATCCTTTTATCGAGGAAGCCATAAACGGCGCGGACAATATCGAGAAACTGTATGTCGCGTACCTG |
| | | AAAAACGAGAAGAAACATGCAGAAAAAATCTTACGAAATATTGACGAACACTTGCAGTCTTTGCCGCCTTTTTT |
| | | GAGAGCCTCAGATGAGCAAAATCATTGGGGTGTGGATCTTGAGCAGCAAGCATATATTAACAAGTACGCCGCG |
| | | AAACTTAGCGAATTACCTATACAATTGCCAGACGGTTTGTTCACCAAGTACATTGTCGAATTACTGCATAATGCC |
| | | ACTAAAAAATACAGCGAACTGAAAATCATGGATAGCATTGTTGATTCAGATAAAAGAACGACGGATAAAGGCG |
| | | GACATAAAATTGGCGATAATCCGTCCTCTTCCGCTAGTCATATAATTGCGTGCTATTTCGAGAAAGTGTTAGGAG |
| | | ACAAAAGTCAGAAATTTTACGATAGTCAGACGAAAGACTATAAGCGCTTCTACGAGAATATCTCTAAACTGAAT |
| | | CCCAGACGTGAACGCAATAAAGCACTGATACCCGATTATTTTACAGCTAAAGAGATCCAGGAAAAAATCAAAG |
| | | AAGCTCGAAAAAGTGTAAAAGGGAAAAACGATGACGATGCTACTACTCTGAGAGATAAGCTTAATCAAGTCGA |
| | | AGAAAAAGAACGCGCAATACGGCTGTTCCGGGTGGAAGATATGGTGTTGTTTCTGTCAGCGAAACAAATGCTGA |
| | | TGTCCGTCGTTGGCCGCTCTTTAACAGAAGACGTTAATCAGACAAATGCATTACATGAGTCGAAAGATAATCTTT |
| | | CTCAGCTGCGTATTAAGGCAAAAGAGCTCAAACTGGGCCAATTTGGTTTTGATGAAGATTTTAGATTCTTATCTG |
| | | AAGGAGATAATGAAGAAGAACGTAAAGAGAAAGATCAGACCGAATCGTTGCTGGAGTACATCTATCCTTATAA |
| | | GATACGAAAGGAAGGAAAGCTTGTGGGTACGATCAACATAAAACAGAAGGGACTGTCACTCAAAAATTATGGC |
| | | AACATCTATTCGATTCTCGGTGATGATCGTCTGAAGTCACTGATGGAAGGACTCTTTGAAATCGGTGTATCTGAG |
| | | GTGACCTTTGGCGATATTACATCCGAATTCGCAAACTTTGACCAGCTGCGTTCCACAATTTTAAAACTGTTGCAG |
| | | GACAACATAGAAGAGATTGCTTTCAATCGCAACATATCGGTACTCAACAATCCGCGTAATCGTAACTTCTACTCC |
| | | GTTAAGGATAAAGACGGGAACCAGCTGAAAATCAAGATAACAGACCCAGTTACGAACGAAATTATCGCTCTGT |
| | | CCAAAGATGCTATCCGGAATCAGTTTCCCGAGCTGATTAAAATGTTAGAAGAATATGAAGCGGATCGCGTGAGC |
| | | ATTAATGAACTGCGCAAGGCTGTGGCCCACAACCACTACCCCAAGGACATTTCGAAGTTAAAGCCGACGAAAA |
| | | ACAAAAAGCTCCAGCTGCCGAACATAGCCCACCTTGCCAATGAGAACGCAAAAAAAATTATAGATGCTGCTCA |
| | | GAAAGCGTCTGCTGAGAAAGAAAAGAAGAGCGAGTAA |
| 349 | JW13174 | ATGGCTAATACGCAGTTCAAACTGAAATCTGTTGATTATAGCAAGCCTTACGATGAACAGCCTGAGTGTAAACG |
| | | GCATGTCGTGGGAGCCTATGCAAACCTGGCCCGACATAACATGACCCTGACCGTGAATACTATAATGCAGGCCA |
| | | TAGGCATGGAAATTTTCGATGAAAATAACATCGGTGACGCATTCAATTCCAGTCATCGAAAAAAAATTGACGCC |
| | | CTCGACAATATTCAGAAAGTTAACCTTCAGAAACGTCTGTATCGACACTTTCCGTTTTTTAAACGTATGAAATTA |
| | | GAAGATGAAGACAAGAAGTCTGTCCAATTGACCACTCTCCTCGAGGTCATGAGCGATTTTACCTCTTGTATGGCC |
| | | ATCATCCGGAATTTTTACACCCATTATCATCCGTACAACAGCCCAGATGAAAAAAAACGTCAGCTGGAATTGAA |
| | | AAAGCGTATGGGGAAGAGACTGCAGTTTCTGTACGAAAACACCTCTCAAATGTTTAAAACTACGGAAGCGCTGG |
| | | ATCACGAATCAAATGAAGTATTAAGCGCGTTGCGGATTCCCGAGGATTACATCGAAGAGATACTTCCGGGCGAT |
| | | AAAGAGTATGATAACCTTTTCAACTTGATTAACGACCCTAGAACCTCGAAGTTTAAAAAACAAGGTCTGAAACT |
| | | GAACCAGCAAACCAAAGTGATCACACGTAAAAAAGTTCGTTTTGTGCGTAATCCGGACTATCAAGCTTATATGA |
| | | TGGACGAAACCAACGGAATGTCTGATGTAGCAACTATCTTCTTTCTGTGTCTGTTCTTAGATAAAAAGGTGTCCT |
| | | TTGACATGATGGATGAAGTTGGTTTTACCAAACAGATTAAGTTCAATGGTCCGCAGGCTGACCGCCAGTTACTGT |
| | | ATCTGAAAGAAATCATGTGCATGAATCGAATTAGAATGACGAAAGCGAAACTTGATTCTGAAATGACAGATAC |
| | | GGCTCTCGCGTTAGATATGCTCGGTGAAGTTCGCAAGTGTCCTAAAGCCCTGTATGAAGTTTTAGATAAAGATGC |
| | | CCGTAATGAATTCAAAGATGATGCGACAGTACAGTGGGAAACTGAGCACGGCAAAGAAGCCGTGTCCACTGAA |
| | | GCATTAGCGTCGGAATCAGATGAGGATGAATTAACTGATAAAAATACGCCTCGCTCAACATTTGTTCGGTGGGA |
| | | TGATCGTTTTCCCCAGATGGCGCTTCGGTATATTGATTATCAAGGCCTGTTCAATGACATACGTTTCCAACTGAA |
| | | CTTGGGCAAGTATCGTTTTGCCTTCTACCAACACGATAATAAACAGTCAGTTGATAACGAGGAGCGCTTGCGGA |
| | | TTTTACAGAAAGAACTGCATGGTTTTGGACGTATACAGGAAGTCGAGGAGAAAAAGAATGAAAAATGGAAAGA |
| | | ATTATATGATGAGAAGACGGTGAAAGATGGGCTGACCCAAAAAAAGACAGATGAAGCAGGGCAGGCGCCGTAT |
| | | ATTACAGAGCAATACGCACAATATGCGATCGATGAGAAATCGCATTCTATCGGCCTGCGCTGGGAAGGTTGGAA |
| | | TAAGAAAGACAAAAGTAAAGGTCACTATGGTGATCTTGATAAACTGAAAATGTTCATCCCATACGTACCATCCA |
| | | AACCGAAAGAAGAGGAACGGCAGACTAACCAGGCCGAAGATCTTATGGCCCCGCAAGCAATGTTAAACCTGTA |
| | | TGAGTTACCAGCTATATTGTTTTATCAGTATCTTCTGGATAAGTACAAGAGTGATACTTCCAACGCGGAAAACAT |
| | | TATCAAAACCGCATACAGCAATCTGGTCAGTTTTTTCAACGATGTCGATCAGGGGTTATTGGCTCCCGCCGAGGA |
| | | TGCAGATGAAAATGAACGCATTAGTAAGATATCCAGCTTATTAGAAGGATACCATCTTCAAATGTCAGATATCC |
| | | CGGAGAAGCTGCGCAATTATCTTTGCAATAAAGGGATTGACTATGATTTGAAAATGGAGAAATCAGCAATCGGA |
| | | CGCCTGGAAGAAAAGAAAAAGCACGTCGAAAAAGCACTGTCCTCGTATAAAGACAAAAAGAAGATAATTGGAT |
| | | CCAAAGACAACAAGTTTGACAAAATGAGAGCTTCAATTAAAACCGGTCAGCTGGCCACTTGGCTCATTCGCGAC |
| | | ATCATGGATTGGGTCCCAAATAAAAGTTCTGCTCGTAATCGTTTGACAGGTCAAAATTATACTGCGTTGCAGGCT |
| | | GCGATGGCGTTATTTGGTCAGCAAGCGAATTCAAATAGAAGTTATCTGGAATCATGCTTTCGGAATGCTCACCTT |
| | | CTCCCGCTGGATGGCAAACCGTTTAACAAAAATACACATCATCCGTTTCTCGAAGACGTCCTGCAGGCTATGGA |
| | | AGACGAGACGTTAGAGTCGTTCTATGAGAAATATCTCGAGTTTGAGACCAAGCATATAGCCAAAGTAATCAAGA |
| | | CACTGAAAAACGGCACCGATTTTGACGATTATTATCAAGTTCCTTTTCTGCATTGTGATCGTCAACGCTGGGCGG |
| | | GCCCTGATAAAAATCAGGCAGCCCAATATCTTAAACGCCCACTGCAATTGCCTAATGGCATTTTTACCCAGCCG |
| | | ATCTTTGATCTTCTGTGGAAAATCGATAATCTTGAACTCCATAAAGCATTAGAAGAGTCACAGCAGGCCAATGG |
| | | TGACAATCCGCCCTTAACTGATAACGTGGCTTACCTTATGCGCTTATATTTTGAGTTTGTGGAACATGATCACGC |
| | | GCAGCCCTTTTACTACACCACTACGACGGACGGGAATAAAAGTCATTACCTTCACATCTACCGAATTTTTAAGAA |
| | | ACTCTACGGACAACCCATTCCACATTCGAATAAAACAACTGTGCCTGCATATACAATAGATGAAATTCGTCATCT |
| | | GCGGAAACAAGCTATTGCCGATATTGAAGAATATGTTGACAATGAAATTGAGAAATGGCGCCGTACAAAACAG |
| | | GATCGATTCGAGTCGAAACTTAGAAAAAAATTGAAGAAAGAGAACGACAAGCGGTATAAGAAGCACGAAAAG |
| | | CAGTTAAACATAAACGAAGAGATTCAGAAAGCCGTGAAAAAGGAAACCGATACGATGCGTACAGATATGACAA |
| | | GCAAAATGAAACGTCAGTTGAGAAATGTGTATGACAATGAGAGAACCATACGTCGGTTTAAGGCACAGGACAT |
| | | GATGTTACTCATCATGGCAAGAAATATTCTGAAGGCGAAAAGTAACGATAAAGACTTCACTGATGGCTTTTGCC |
| | | TGGAAAATGTGTTCAGCGACAGCTTACTTGATAAACCAATTGACTTCGAATGGAAAGTTAACATAAAAGGTGCT |
| | | TCTAAGACGATCAAACAGGAAGGTATGAAAATGAAGAATTACGGACAGTTTTACAAGTTTGCTTCAGACCATCA |
| | | GCGACTTGAAAGCCTGTTAAGTCGCCTGCCTGATTCTCTGTTCCTGCGCGCGGAGATTGAGAATGAATTAGCATA |
| | | CTATGACTCTAACCGTAGCAAAGTTTTTCGTCAAGTTTATATCATAGAGAGCGAAGCTTATCGCTTAAAACCGGG |
| | | CTTGCAAGATGACGCAAACGCCAGTAAAGACTGGTTTTACTTCACAGATAAAAAAACACACAAACAGCGCCCC |
| | | ATTAGAAATAGCTTCATTCAGCTGCTGGAAATTCTGGCCGCGGGTGAAGATGGGATTTTAGATAATGACGAGAA |
| | | AGTTGCACTGCAGAAAACCCGCAACGCATTTGGCCATAACACGTATGACGTAGACTTGAATGTAGTATTCAAGG |
| | | GGAAAGAAGACAAAAAAAAGATACCAGAAGTAGCTAATGGAATTAAAGAGAAGATTGAGACGCAGACGGAAG |
| | | AACTCAAGCGCCGTTCCAAATAA |
| 350 | JW13175 | ATGACAGACAACACTAACAAGTATTTTAAGCTTAAAGAGGTGGATCACACACAAACGTATGAAACGCAGACAG |
| | | ATTGTCAACGTCACGTCGTGGGCGTTTACGCCAATATCGCTCGTCATAACATGACACTGGCAGTCAATACTGTTA |
| | | TGCAGAGCCTTGGTGAACCATTATTTGAAGAAAACAGTATAGCAGATGCCTTCAATGATACGCATCGCAAAGAC |
| | | ATTAGAGATTTTGAAAACATTAAGAAACTGGAGCTGCAGGAACGCTTGTACCGTCACTTCCCTTTCTTGAAACGT |
| | | ATGTACAAAGATAAGGAAGTATCTCTGAAAGATCTGATGGAGACGATGAGCTGTTTTACTCAGAGCATGGCTAT |
| | | GGTCCGGAATACATATACTCATTATCGTCCGTTTGAATCTAATGTCGAGGAACAGAACGATCTTAAAACTAAAC |
| | | TTGGACAGTATATGGAAGACCTTTTTCTGGAGTCAAGAAAGGTCTTCAAAAAAGAGGAGCGCCTGGACGCCTCT |
| | | TCCAACGAAGTCTTGGGTAGAGTGTATAAGTCGATTAAAAATGACAAAGGTGAGAAGTATGTTCTCGACTTTAA |
| | | CTACCCAGCGTATATGAAGCAGGGTAAAGAAGGACTCAGCGATGTCGGGTTACTCTACTTTCTGTGCCTGTTCTT |
| | | AGACAAGAAAGCTGCCTTCGACCTTCTCAACCGAACCGGCTTTACGCAGCATGCGAAGAATTATTTTGACGGGA |
| | | ATGAGGAACAGACCCTGTTGTTACTGGAAATGATGTGCATGAATCGGATCCGTATGGTACATAGCCGTATTGAT |
| | | TCTGAAATGTCAAACGAAGCATTAGCGTTAGACATGTTAAACGAACTGCGAAAGTGCCCACGCCCATTATACGA |
| | | GCAATTAGGAAAAGATGATCGTGATTCTTTTAAGGATGTAATGTCAACACAGTGGGAAAACGAATCTCCGGATA |
| | | ATGAAGACGCACCCATTGATGCCGAAGGCAAAAGTATCCCCAAAACTACTATGGTTCGATGGGACGATCGATTT |
| | | GCAACGATGGCCCTCTCCTATATAGATACAATGCAGTGGATGGATGACATTCGTTTCCAGCTCAAACTTGGTAA |
| | | GTATAGATTTGCATTCTACAACCATGACGGAGACCATATAGCTGGAAATCTGTTACGAGTCTTACAGAAAGAAA |
| | | TGCACGGATTTGGTCGTATCCGGGAAGTAGAAAAAGAGATGAATGAAAAATGGGGCGCCCTGTTTGAACAAAA |
| | | ATATGAGGAGAATGGGCTGGAGCGCAAAGATGCAGATCAGGCGGACCAAGCTCCGTATATTACTCGCCAACGT |
| | | CCTCAGTATGCGATAGATGACGAAAGCCATAGTATCGGAATGCGGTGGGATTTCACGCGTCCGAAGTATTGGGA |
| | | TTTTACGAAACCTAGTTACGGGGATCTTGCAAATAAGAAAATGTTTATTCCGCATCTGCCAGGCAATCCGAAAC |
| | | CTGCGGAAGAGGGTAAACATCAGACAAACCAAGCTGAGCGGCTGATTCCGCCGCAGTGTACCATGAGCCTCTAT |
| | | GAACTGCCAGCTTTACTGTTCTATGAATATCTTCGGCGGAAACTTCGCAATGAGACGATGGATTCAGCCGAGCA |
| | | AATTATCAAACGTTATTGTAACTCCTTGAAGTCTTTCTTTGGCGACGTATCTAGAGGTAAAGTGTTTCCGGATGA |
| | | TGAAAACGAGTTGGCCGAAGCTCTGAACAAAGATTATGGGTTGAGATTGTCCGATATACCCGAACGGTTGAAAA |
| | | ATGTGCTCTTAGGCAATAGTTTCGACGGTAATGAGAAGCTGAAGAAGATAGTTAAGACCTTCGTGGAAGAACGT |
| | | AAAAATGAAATTAAAGAGATGAAAAGATGCTTAGACGAGGGTATTAATCATAAACTGGTTAAAACCGGTCAAG |
| | | CGGCACAATGGCTGGCGCGTGACATTATGATATGGGTACCGACACAGACCCGCGAGAAACTGTCGGGGCAAAA |
| | | ATATTCAGCCATTCAGGCCAACCTTGCGATGGTTGGTAACTGGGAAGGGGCTGTGAGTTACGGTGATCTTCGCC |
| | | AGCTGTTCACCGCAGCAGGCATAATTCCAGAATCAAACCTGAAAAACCCTAATCAGCATCATCCTTTCCTGAAA |
| | | GATGTATTAAACGGATTATTAAACGATCGCGAGCATTCGGTTAAAAGTCTGTATCAACGGTATCTTGACGTTGA |
| | | AAGCAGCCACATATCGTCACTCGAGTTAAAATTAGCGAGAAACAAAAATTGCCTGGATTCCTTTCCGTCACTTC |
| | | ACCACAATCGTATGCGTTGGAAACAGCAGGATGCGGCCGCTTTAGCGCAGTCGTACCTTACAGTCAAAAATAAG |
| | | GCAAAAGATAGCGAGATCATTCTTAACAGCGAGAAACCTATACAACTCCCCAAAGGCATGTTCGCTGAACGCAT |
| | | CTTAGCCATGATGAAAGAGTTGGCGAAGAACAATGGCGCCCTGAACGAGGGTTTAGAAAAAATCGAGAAGCAA |
| | | GAGAAGGGGTTAAAAGGTAATACCGCATATCTGATCGGTTTGTATCATACTTTTGCTGAAAATGATGCCTCTCAG |
| | | CCCTACTACAATTATGAACGCGTTGTTGACGAAGGTAAACTTGCAAAAAGTGAACGTAAACGTATGGAACAGAC |
| | | GATTCGTCGCTACAAGACTCAGGATATCTTGCTCTTTTACATGGCTAGAGAAATTCTGGCTTTGCCGGTGGGAAA |
| | | TGACAATCGGCAGCGATTTCTGTTGAAGAACGCCATGAATAATGATCTGCTTAGTTTACCGATCGACTTCTCCTG |
| | | GACCGTTCGCTTAAAAGATGGCTCTGCGAAAACCATTACCCAAAAAGCGATGAAGATGAAAGACTATGGAAAT |
| | | TTCCACAAATTTGCCTCGGATGGTAAACGTCTGGCAAGTCTGCTGGAACAGCAGGCTGAGACCGAATTTGCGCG |
| | | AGCAGATATTGAAGCGGAACTGGCGCGCTATGAACAGAGTCGGGTGGAAGTTTTTAGACAAGTGTATATTCTCG |
| | | AAAGCCATGCCGTAATGAAACTGGGCGTGGCAGATGATTGTGATATCAAAGCTCGCGAAGATTTGTTTTACGAT |
| | | TCCGCAAAAGGCAAACGTTGCAAACTGAATTCCTTTAATCAACTGATGCAGGCACTGGGAATCGGGGAAAAGG |
| | | ACCGCGAATCTCTTACCAAAATTCGCAATGCCTTTGCGCACAATTACTATGTAGATCTGAAAGGCATCGGCTTTT |
| | | GGGGCTCAGTTCCCAAAATTCCTAATGTGGCATCAAAAACAAAAGAATGGATGGTGCGCAAGTGTAATGATCTG |
| | | TATGGTCCACGTTTTGGCGGGTAA |
| 351 | JW13176 | ATGGCCAAAAAAAAGACCGCAAAACAAATCCGCGAAGAACAAAAAGCCGCAAAGATGCAGAAAATTCAGACC |
| | | TTTGTGAAGGCTAAGGAAGCTAAGAAAGAAAATGAAAAAACCGCTTCAGCTGTTAAACTTCCGGTCAAGAAAA |
| | | ATCGTAAAAAATCCCTGGCCAAAGCCTCAGGTTCTAAAGCCGTTCTGATTGTAGGTAACGATATTATTATGACTA |
| | | GTTATGGTGAACGCTGCCTGGCGAATCTGGAACATAAAATTACTCCCGAGCTGAACACCGAAAGCCTGACGGTT |
| | | AACAACGTTCTGAACAACGTCAATGCGAAAGATGGTAAAAACATCTCGTTTACAGGGCGCATATCTGGTTTATC |
| | | CGTCTCTTCAGACAATCCACTGCATTTTCATCAGACTGAAGAACGTAAAGCCCCAAAACCAGGGACTGATCTTTT |
| | | ATGCTTAAAAGACAAACTCGAACAGTGGTATTTTGATAAAACCTTCGACGACAATATACATATACAATTGATCT |
| | | ACCAAATACAGGACATAGAGAAGATTCTTGCGTCACATGTCAACAATATTGTCTTTGCACTGGATAATATTTTAC |
| | | ACGCCACAGAAAGCGAAACAGGCGATTTTCTGGGGATGGGTTATATGCGTGCAACCAATACCTTTGAAGAGTAT |
| | | AGAGACAGTGCGATTAGTAACGTCAAGAAATCGTATGAAACGTTCGAGACATACATCAAAAAGAAGGAGTTAC |
| | | TGTATTTTGGAGAAGCCTTTTATCATGGCAATACGTGTCGGAAACCCGAAGATATCTATCACATTCTTGCAATGG |
| | | TGTCATCCTTGCGCCAGTTTTGTTTTCATAGCACTTTCGATGATAAAAGTAAGAGCATTAAGGGATCTTGGCTGT |
| | | ATCAGCTGGATGAACAGCTCTCAGATGAATTCAAGGAGACCCTGGGTATTTTATGGGCTGAGGCCACCGAGAAA |
| | | CTCGACAGAGACTTTCTTAAAACAAATACGGTGAACCTGCATATCCTGTGTGAGATCTTTGATAAAGAGAAGCA |
| | | GGAAGACATCATCAATGCGTACTACGAGTTTTTGATGAAGAAGTCCTTTAAGAATCTCGGGTTCTCAGTTAAGA |
| | | AACTGCGGGAATGCATGCTCGAGGAAGCAGAGTTAAACGAATTCAAAGAAGATAAGTATAACTCGGTTCGCTC |
| | | GAAACTGTACAAGTTATTCGACTTCATCTTGGTGCATCACTATAAGAATGATAGCTCCCCGGAATCTACAGCTTT |
| | | GAAAAATTTGTTACGGACCTGTCTGACGGATGAGGAGAAAGAAAAAGTGTATGTCCAGACCGCCAAAGAGTTA |
| | | ACCGCGCTTTATTCCTATCATTTTAAAGAAGCTTCTGTATCTGTGAATGCGCGTAATATCAAAGAATATCAGGAA |
| | | AAAGCTCAAGTGTATCACATTTCGATTGCTGATAAAATTAGCTCTACAGGTATAGGGCTGTTTAGTCAGCTCATC |
| | | TACATGCTCACCTTGCTGCTTGATGGGAAAGAAATTAACGATCTTCTCACGACCCTCATCAACAAGTTCGACAAT |
| | | ATAATCGGCTTCTTAGACATACTGGACAAAATGCACCTGGATACTGAGTTCAAAAAAGAGTATCAGTTCTTTAA |
| | | CCAGGAGAATTGCAAGTACACGGCGAAAGAACTTAGAACCATTAATAGCCTGGCACGTATGCAAAAACCAAGC |
| | | GCTAATGCAAAAAAAGTAATGTTTCGTGATGCGCTTAGAATTTTAGGCATGGATAATCGATCAGATGAAGAAAT |
| | | TGATAAAGAATTAGATCGAACAATGCCTGTAGGAGCAGATGGCAAGTTTATTAAAGGCAAACAGGGTTTTCGGA |
| | | ATTTTATTGCCAGTAATGTCATAGAATCATCGCGTTTTCGGTATCTGGTGCGCTACAACAACCCGCATAAAACGC |
| | | GTGAGTTAATTCAGAACCAACATGTTGTGAAGTTCGTGCTGGAAGGAATACCGGACACCCAAATCAAACGGTAT |
| | | TTCGAAGTCTGTAAAGATAAAGATACTCCTGATACATCTTCCAAAAGTGCCCAGATTGACGTTCTGGCAGGTAT |
| | | ATTGACAAACATTGACTACAAGATATTTGAAGACGTTCCTCAAAGCGCAAAAATCAATAAAGACGATGCGTCTC |
| | | GTAATCGTGCTGAAGCGCTGAAAAAACAGCGTTATCAAGCAATTGTTACTCTGTATCTGACGGTGCTTTATCTGG |
| | | TGACGAAAAACCTGGTGTATGTTAACAGTCGATATGTAATGGCCTTTCATTGTTTAGAACGCGATGCGCTGTTAT |
| | | ATGGCGTTGCATTTCCGCCAGTTGAAAAGAAATGGGGTAAAGATTATTCACAGTTGACAGCGTCATTGTTAAAA |
| | | GATGAGACTTACACAACTTATGGCCACCTGAAAAATCAAAAAGGACATCGAAAGTGGCACGTGCTGGTCAGCC |
| | | GCAATCTTGAAAACTCCGACAAAAAAGCTGTTCATTGGTTCAGAAATATTAGCGCACACATCAGCGTAATTCGC |
| | | AACATTAATGAATACATCGGCGAGGTTCAGGAGTTTCACTCTTACTTTGAGCTCTACCATTACCTTGTACAAAAA |
| | | ATGATAGCAAAAAATAATTGGTATGATGCCAAGAGTCAGCCCAAGACGGCCGAATATTTGGAGAATCTTGAAA |
| | | AATACCACACATATAGTAAAGATTTCGTAAAAGCGTATTGCATTCCGTTCGGATACGTAGTGCCGCGTTACAAG |
| | | AACTTGACTATCAATGAGTTATTTGATCGCAACAATCCTTTGCCTGAACCGAAAGAAGATAAAGAAAAAAAAGA |
| | | GGTTTAA |
| 352 | JW13177 | ATGAAAAAGCGCTTGAGTAATCCGCCAGCCCGCCGCCAGGATTCAAATCCCCCTTCTGCCTGTAAACTTTATCTT |
| | | ACAAGCTTTGGCAAAGGAAATGATGCAGTGTTAGAACAGCAAATTGATACAGCTGCAGACTATGCCGTGTCACC |
| | | GATTTCTGCTGAACCATCATTGACGGTGACTGAAGCGGCAGAGGCGCGCGTCAAGTTTGATTCGAAACGACCTA |
| | | ATGTTAAAAAAATCCAGCTCACCGCAGACAATCCTCTCCATCATTCCGCGGGAAATCCGGCGTCAACGCGCCGA |
| | | GACATTCTGGGCTTAAAAAATGCCTTAGAAGAACGGTTTTTTGGCCGCACCTTCGATGATACCATCCATATACAG |
| | | ATCATCTACAATATACTGGACATAGAGAAGATTCTCGCCGTTTATGCAACAATTATATCAGCGGCCCTGAACCAT |
| | | CTGATGGATGAGTCCAGCGATGTGTCTCAGAGTGATGTCATTGGGAATATAGCTACCCAAAACCCTTATGCCATT |
| | | TTTAAAGACCCTCAGTCTAGATATGATTATCGTGATCCGTCACTGACGCCAGATCAACGCAAAGATGCAACCAG |
| | | AATTCGCAAAAAGATTGACGCAAATCGGGCGGCCTTTCAGGCACTGTTAGCGACAGGCCGTCTGGGCTACTTTG |
| | | GGTTTGATTATAAACCGCCGCGTAAAGATGAGAAAGATAAAAAAAAGATTAAAGCAGCAGAAACGCAAGAGA |
| | | AGAGATTGTATCATCTTGTCGCCCTCGCGGGTCAATTACGTCAATGGTCTTTTCATGGTGGTGATAAACCAGAGG |
| | | ACGAACTGTGGCTGTATCAGCTGGATAGCCAGCTGGTTGGCGAGCGCAAAGAGTTCCTGGATACATTGGACTAT |
| | | TATTACGGCGATCGTTTCAGAGAGATCAACGACGAATTCATCGAGCAGAACAAGATCAATTTGGCTATACTCGA |
| | | AGAGTTGTACCCCAAGGAAGACTTTCCCGAGATCGCAGCCCTGTATTATGACTTTATTGTAGTTAAGAGTTACAA |
| | | GAACATGGGATTCTCTATCAAGAAATTACGTGAACAGATGACTACTTTAGAAGGTGCAAGCGTGATAACAGCTC |
| | | AGAATATGGACAGCGTGCGGAGTAAACTTTACAAACTTATCGACTTCAGTTTATTTTACCGCTACTACAAAGATG |
| | | AGGCTCGCCGCCAAAGAGCGGTGAATATACTGCGCGCAAGTACCACAGATGAAGAAAAAGAGGCTTTTTATGC |
| | | TGCTGAAGCCGAATGGAACTGGAACTATTTTCGTGGTCGTTTTACGGATTTTTGCCTGAAAATAGGTGATTGGGT |
| | | AAAGATGGATGTGGATGCAAGCCGTTGGAATGGGATATTAGACCTTGATAGCTATAGACGTACCAGCTCCGCGT |
| | | CTTATTTTTCCAAACTGTTGTACGCTATGTCGTTTTTCCTTGATGGAAAAGAGATCAACATCTTGTTCACCACGCT |
| | | CATAAACAAGTTCGAAAACATTGCGTCTTTTATCACTACGGCCAAAGAACTGAGCATTGATGTCACCTTCCACG |
| | | AAAACTATGCTTTCTTTAATCAGGATTGTGACGGATATGCTCGTGAGATCGATGTTGTGCGAAATATTGCACGGA |
| | | TGAAAAAACCAGTTCCAGGTGCCCGTAAAACCATGTATCGTGATGCCTTAACTGTTTTAGGTATTCCTGAGCACA |
| | | TGCAGGCAGATATGTTTGACGCAGAACTGGAGAAAATGTTAGAAAAACCGAAGGACGAAAAAGGGCGGAAACT |
| | | TAAAGGGAAAAATCCCTTTCGCAATTTCATCGCAAATAACGTTATCGAATCCACTCGTTTTATCTACGTCGTTAA |
| | | GTTCTGCAACCCGGATAAAGTTCGATCCCTGGTAAACAACACAGTTGTGACTAAGTTTGTCCTCGGGCGTATGCC |
| | | AGAAACGCAAATTGACCGTTATTACCAATCTTGCATTGCCAATCCGGATAGCGCTGCCCCGCTGGCTGCGCGTAT |
| | | AGATGCGCTGGCCGATATGATGCGTAATATGAGATTTGAAGATTTTAAAGACGTGCAGCAAAAATCAGGTGATC |
| | | CGATTGAAAATATGAGAAAAGAACGTTTCAAAGCGATTATCCGACTGTATCTGGCAGTAGTCTATCAGCTGGTA |
| | | AAAAACCTTGTGAACGTTAATTCTCGGTACGTTATTGCGTTTCACTGTCTGGAACGTGATTGTCAGATCTACACG |
| | | GGAAAGAGTGTCTCCAAGTCCAAGAAATATTTTACTCTGATTGACGTGTTGCTCGAGGAAGGTGATTCGAGTCG |
| | | CAGCGGATATCTTGCCCGCAATGTACGGATGCGTGAACACATTGCACATGATGTAGATATTGGCAAAAGTCTGA |
| | | TTATCACTACCAAAAGTACCGACTCGGCTACAGGTGAAGTTAAGTATTCAAAACTTCGGATTATAGGGTTCTATC |
| | | GAAATAACATTGCGCACTTGACAGCAGTTAGATCATTTGCTGACTACATTGGTGATATCGCGCGCATTGACTCGT |
| | | ATTTTGGCTTATATCATTATATTGTCCAGCGCTTACTCCAGTCTCGAGATTACACAGCGGAAGCCTTCCCAGATC |
| | | GGATTAATCCTTACTATGCGTCATTGGACAGATATCATACCTACGTAAAAGATTTCGTAAAGGCCCTGAACTCAC |
| | | CCATAGGCTATAATCTTCCTCGGTTCAAAAACCTGAGCATTCAACAGTTATTTGACCGGAATGAACTGCAGCCG |
| | | GAGCCGAAAGATGACAAAGCTGAAGAATCGTAA |
| 353 | JW13178 | ATGGCGAAAAAAAAGCGTATGAGTGCTAAAGAACGTAAACAACAACAAATTAATCTGCGAATTAAAAAAGCCA |
| | | CCGAAGATTCCACCAAAAAAGTTAACACGACGGTCGCTGTAAACAATAAACCGATTTCAAAAGAGATCAAGAA |
| | | ATCGAAAGCAAAACTGGCTGGTGTTAAGTGGGTGATTAAAGCGAATGATGATGTCGCTTATATTAGCTCTTTTG |
| | | GTAAAGGAAACAATAGTGTGCTTGAAAAACGTATTATTGGGGATGTTAGTTCCGATGTGAATAAAGACTCTCAC |
| | | ATGTATGTGAACCCGAAGTATACAAAGAAAAATTATGAAATCAAAAATGGATTTTCCAGCGGAAGTTCTCTGAC |
| | | AACGCATCCAAATAAACCCGATAAAAACTCAGGCATGGACGCTTTATGTCTGAAGACATACTTTGAGAAAGAGA |
| | | TCTTCAAAGACAAGTTTAACGATAATATGCACATCCAGGCAATCTATAACATCTTTGATATTGAAAAAACTTTGG |
| | | CCAAGCACATTACAAATATAATCTATGCGGTTAATTCTCTGGACCGCAGTTATATTCAGTCAGGAAATGATACA |
| | | ATAGGTTTTGGTCTTAACTTTAATATACCGTATGCAGAATATGGCGGCGGTAAAGATAGCAATGGTAAACCCGA |
| | | AAATAAAAGTGCATGGGAGAAACGTGAATCCTTTATTAAGTTTTACAACAATGCTAAAGATCGCTTTGGGTATT |
| | | TTGAATCTGTATTCTACCAAAACGGTAAACAAATTAGCGAGGAGAAATTTTACATCTATCTCAACATTTTAAACT |
| | | TCGTCCGGAATAGTACATTTCATTATAATAATACTTCAAGCCATCTCTACAAAGAGCGGTACTGTAAAATCAATC |
| | | CTAAGAACAATTTGAAAACCGACTTTGAATTTGTAAGTTACCTTAACGAGTTTGTCAAAAATAAGTTCAAGAAT |
| | | GTCAATAAGAACTTCATTAGCAACGAGAAAAACAATCTGTACATAATCCTGAACGCTTATGGGGAAGACATTGA |
| | | GGATGTAGAGGTGGTAAAAAAATATTCCAAAGAACTGTACAAACTGTCAGTTCTGAAAACTAATAAAAACCTG |
| | | GGTGTGAATGTCAAAAAATTACGCGAATCTGCGATTGAATATGGCTATTGTCCGCTGCCTTATGATAAAGAAAA |
| | | GGAAGTGGCCAAACTTTCATCCATTAAGCACAAGCTTTACAAGACGTATGATTTTGTAATAACTCACTATCTTAA |
| | | CAGCAATGATAAACTTCTCCTGGAGATAGTGGAAGCCTTGCGGTTGTCGAAAAATGATGATAAAAAGGAGAAT |
| | | GTGTACAAGATCTACGCCGAAAAAATCTTCAAAGCCGAATACGTTATTAATCCCATTAAAACCATCTCAAATCT |
| | | CTTTGCAGAGAAAGGTGATAAACTGTTCAATGAGAAAGTATCGATTTCTGAGGAATACGTCGAGGATATCCGTA |
| | | TCGACAAGAATATACATAATTTTACAAAGGTAATCTTCTTTCTTACGTGCTTCCTGGACGGGAAGGAAATAAAC |
| | | GATTTGTTAACCAACATCATCAGCAAACTGCAGGTGATAGAAGACCATAATAATGTTATCAAAGCCATAGCCAA |
| | | TAACAATGACGCGGTGTATAAAGACTATTCAGATAAGTATGCGGTTTTCAAAAATTCTGGCAAAATAGCTACCG |
| | | AATTAGAAGCAATTAAGAGTATTGCACGTATGGAGAATAAAATTAACAAAGCCTTTAAAGAACCATTGTTAAAA |
| | | GATGCGATGTTAGCGTTAGGCGTTTCCCCTAATGATCTTGACGAGAAATATGAAAAATATTTTAAAACCGATGTT |
| | | GACGCAGACAAAGATCACCAGAAAGTTTCGACCTTCTTAATGAATAATGTCATTAACAATTCGCGCTTCAAATA |
| | | TGTGGTCAAGTATATTAACCCAGCGGACATAAACAGACTGGCGAAAAACAAACATCTGGTGAAATTCGTTCTCG |
| | | ATCAGATCCCGCATAAACAGATTGATTCGTACTATAACTCAGTGAGTACGGTTGAAGAACCAAGCTATAAAGGC |
| | | AAAATACAGCTGTTAACTAAAAAAATTACCGGCCTGAACTTTTATTCTCTCTTTGAGAATTGTAAAATACCTAAT |
| | | GTGGAAAAGGAGAAAAAGAAAGCCGTGATAACACTGTATTTTACTATCATCTACATCCTGGTAAAAAATTTGGT |
| | | TAACATTAACGGCCTGTACACGTTAGCTTTATATTTTGTTGAAAGAGACGGGTTTTTCTACAAGAAAATCTGCGA |
| | | AAAGAAAGACAAAAAGAAGACAAATAAAGATGTTGATTATCTTCTGTTGCCGGAAATTTTTTCAGGATCTAAGT |
| | | ACCGCGAAGAAACCAAGAACTTGAAGTTACCGAAAGAAAAAGATAGAGAAATTATGAAGAAATATCTCCCTAA |
| | | CGACGAAGATCGCAAAGAGTATAATAAGTTCTTTAAACAGTATCGAAACAACATTGTCCATCTGAATATTATAG |
| | | CAAACCTTAGCAAACTGACTAGCACCATTGATAAGGAAATTAACTCCTATTTCGAAATTTTTCATTACTGCGCAC |
| | | AGCGTGTAATGTTCGATTATTGCAAAAATAATAATAAGGTTGTTTTATAA |
| 354 | JW13179 | ATGGCTTATGATAAACCTCGTCCGAAACGTCGCCAAGCGCGCTCAGATTTCTACATTACAGATAAGGCGATTAT |
| | | GGGCGGCTATTTTAACCTTGCCCAGCTGAACTTCTTTAAGACATTAATGGAGATCTTCACCAAAGCAGGGATCG |
| | | ATGTCTCTAAAATTCGGCAGGATAATTCACCCAAGTATTTGATGATTCTCATAAAGAAATTAAGTCAGGACGAT |
| | | GAAACGATCGATGACAAAAAGTGGGCGGACGCCCTCGACCTTAGTAACGAGTGCTTACTGAAATTACAGCAACT |
| | | GCTGTACAAACATTTCCCATTCTTGGGACCCGTCATGGGTGGCGAAGCTAGTTATAACATCTACAAACTGAAAG |
| | | ATGGGCACCCGGAAGTAAAAGTTGCGAATGACGTGATGCGAGGAGTTAAACTTGAAGATTGTCTGGCGGTGCTG |
| | | CGGCATTTTGCCCTGTGTCTGAACGACTGCAGAAATTTTTACACCCATTTCAATCCATATAACTCGATCGATGCG |
| | | CAAAAAGAGCAGTATTTGTCCCAGAATAAAGTAGCGGTTTGGTTAGATAAGGTGAAAGATGCCTCTCGGCGTAT |
| | | TGTAAAACAGAACAATCAGCTGACCTCGCAAGACATGGAATTTTTGACGGGAATAGATCACATGAAACCACAG |
| | | GACAAAGTTGATGAGTTTGGTAACATAATGACAGACCGTAGAGGACGCCCGTTCAAAGAATTTGTGGAGTATGA |
| | | AGATTACTATTTCCGTATTCGCGGTAAACGGTACCTGGTTGATGCTGCGGGCACAAAACTTGTTGACCAGGAGC |
| | | CCAGAAACGCCCTTTCTGATTTTGGTGTCGTGTTTCTGTGTACCTTATTTTTAGAGAATGACCAGTCACGACGTAT |
| | | GCTGGACGAACTGCGGTTATTTGAAACAGGTCCGTATGATGGGAGCCGGGATGGGGATGAATTCAAAAATGAC |
| | | ATACTGCGAGAGATTCTGCTGTTTTATCGCACGCGCGTTCCGCGGGGCAAACGCCTTGATCCTATGGATGACACT |
| | | ACCTTACTGGCTATGGATATGCTGAACGAGTTGAGAAAGTGTCCTATGCCTCTGTATGATGTTTTAAGCAGAGAA |
| | | GGACAGTCTTTCTTTGAGGATGAGGTGAAACGCCCTAATGATTTTACTCCGGACGTTGCTAAACGCTTACGCTCC |
| | | AGCGACAGATTTCCGTATCTGGCGCTGCGTTATATTGATCTCAATAAGTGCTTCGATAATATCCGTTTTCAGGTT |
| | | CAGCTGGGAAAGTTTAGATATAAGTTTTATGACAAAACCACGATTGATGGTGAACAGGTAGTACGCGGTCTTCA |
| | | GAAAGAAGTTAATGCTTATGGCCGCTTGCAAGATGTAGAGCGTTATCGGCAGGAAAAATATGCCGATATGCTGC |
| | | AACAAACTGAATTAGTAGAAACAGGAGAAGAGGATATAACTATTGCCAATTTTATTCCGGATACTCCACAGAGT |
| | | TCTCCGTATCTGAGTGATCGGACGGCATCGTATAACATCCATAATAATCGCATAGGTCTCTTTTGGAATGTGTTA |
| | | GACGAGCAGGAAGTGCTGACGCGTGAGGAAAAAATGTATCTTCCAGACTTGAAAGTGGATGAAAATGGAAAGG |
| | | CAGATGTATTTCTGCCAGCGCCGAAAGCATCCCTGAGCGTTCGTGACCTTCCTGCCCTGGTCTTTTATCTTTATCT |
| | | GCAGAATCAGCACCCAGATCTGACACCGGCTGAAGCGATTATCCAGCAGAAACATAATGCCTTAGTCCGCTTTT |
| | | TTGAAGATGTTTCGACAGGCCGCCTGCTGCCTGTGAAAGGTATTGCGGAGCTTAAAGCCGTGATTGATCGCTAT |
| | | GAGTATCTTACGATTCACGAAATCCCTGAAAAATTGCGCGATTACCTTGCAGGGGCTGTTGGCACTGAGGATGA |
| | | TGATGATTGCGCCGATCGCTTAGATTGTTATGCGATGGGCATCTTGGAAAAAAGATACCGTCAAGTTCGCGCAC |
| | | GCATTGATCAACTGAAAGAAGCGCGCAAAATGATTGGTGATAAAATGAACCGTTACGGTAAAAAGAGCTATAT |
| | | CGATGTGAAACCTCGGTTGCTGGCTGAACAACTGGCACAGAGTATTGTTGATTGGCAGCCTGCTACAGACAATG |
| | | GGAGACACAAAATGACTGGCAAAAATTTCCAGCGTTTAATAGCTTTCCTCGCGCGGTATACCGACGAAGTACCC |
| | | GGGCACGAGTTAAAAGATCTTATGAGCCGCGCCCGTCTGTTGGACGGCCCAGTTTCCCACCCCTTCCTGCAGCA |
| | | AGTACTGGACAAGAATCCATTGAACATCGAAGTTCTGTATGACCTGTACTTCAAAGAGGAGAAGCTCCATCTCG |
| | | AGAACTATCTGGAAATCGAAACCAATAAACAGGGTGAAGAAGTCTTTCGTAAAAAAGCAAGTGTGGATCTTTCG |
| | | AGAATCCCCTTCCTCAACTCGGGCCGTCTGCGTTTCCATAAACGAGACTCAGCATATTATCAGCGACTGGCTGCA |
| | | CGTTACCTTCAAGTCGATGGTCGGCAGTCCACAATCCTGTTACCTGATGGACTCTTCACCCCGCATATTCTCAGC |
| | | GTGTTGAAGAGCGAATACCATGACCTTCAGGATCATTTGCAGGATCAAGAGTTAAACCAAAATGCAAGCTATCT |
| | | TATGTCTGTTTACTTGGAACATGTTCTGCATGACTCTGCTCAGCCATTTTATCTGTCTAACTCCAATGATTCCCCT |
| | | TTTGCTAGACGTTACGACCTTTTTGCAATCTTGCAAAATAAAAAACGTGGCGTAGAGTTATTACCTTGCCCATTA |
| | | ACGCCAGTGGAGATTAATCGACGACTGACCGGTAAAACCCCGGATGACAATGCGAAAGTGATTGAAAGAGATA |
| | | TCAAAGCCCTCTTGGACGATATGGCACGCAAAACAGAAGAGCGGATTCGCCATCTTCCCCAAGGTTTTTATCGT |
| | | AAAAAAAAGAATCAGTCTCAAGAGGAAATGGCAAAGCTTGAACGTCGATTAAAACATAAAATTATCGACGTCA |
| | | AAAAGAACGAACGTACGATTCGACGGTATAAGACGCAGGATATGGCTATATTTTTAATGGCCAAGCAGATGCTG |
| | | GCAAATCAATTAAGTTGGCAAAACCAAGAAGGGGAAGTGCCGTTCAAACTGGCAAACGTATGTAACGATGGTT |
| | | TTTTGTCACAGACCATGAAAGTCTCCTTTCCGTATGAAGTTCCGCTGAAAGAAGATAAGACCAAGAAGGTTACC |
| | | ATTGACATCGTCCAGGACAATATGAGCTTAAAAAACTACGGGTTACTCTACAAGTTGTACCACGATGAGCGTGT |
| | | CGTCACTCTGTTACAAAATATTGTGGACCAACAGCAGATAAGCTTTGCTGAATTAACGGCAGAATTAAGTTTGT |
| | | ATGACCAGAATCGTTCTAAGGCGTTTTCAAACTTCCAGCAGTTCGAAAAACTGGCGTTTGAAACTTCAAAAGAA |
| | | GCCCTGACTCATCCGGAACACCGGGGCTTTTTCCTTGAAAAAGTGAATAAAAAAAATCAGGTCATTCGTTACGA |
| | | GCGCCGTAACAATTTTGATGCCCTCTTAGATCTGCTGGATGCCATTGAGGCCCATCAATTAGATCCGCGTGAACG |
| | | TCAGTGGCTCACAAGTGTGCGTAACGCATTTTGCCACAATTCATACTACAAGATCGACATGCAACAGATAGAAA |
| | | AGAAGCTCCCCACTATTATACAACAAATAGTAGAAATAGTCCAGCGTATTACCGATAAAGCATCACATCCGTCA |
| | | AACAGATAA |
| 355 | JW13180 | ATGGCGCGCAAACTGTTTTCCAATGAAACCCGGCCGCATCTTGCCGCTTATTTCAATATGGCTTGCCATAATGCG |
| | | TTTGTAGTTTTGAGCAGAATAAGTGAAAAAGCGTCGAAAAGTTGCGACCAGCGGTTAGAGATGAAACTGAGTG |
| | | AGCTGCCCGTCATTTCACTGTTTGATACAAAGAAGCCCGAGGAAAAAGAGTGGATCATGAACCGTTTCGAGAAG |
| | | TCTTTTCCGTTTATTTCCCCAGCAATGAAAATGAATAAAATCAGCGAAGCAGGCTATGATGATCTTCAGTGTTCC |
| | | TCACGGCCGTATGTGCGGACGACGTACGCGATGAAAAAATGCATCGTTGACATTTTCAATATGCTCAATTTCTTT |
| | | AGAGACAAGTATACACACTACGTCTTTAATGATGAGCGAAACGATAACGCGGAATATAGAGATATCCAGAAAC |
| | | GTGTAGTGGAATACCTTGAGGCTTGCTTTACTGCGTCATGCCGCATCGTGAAGGAACGCTTTCTGCTGGATAAAG |
| | | AATTGGAATTCTTAACAAAAGGTCGGTACGAGAGACACGGTGCCAAGTGTGTGGTGAATACACGTAGCCCCTTT |
| | | GCTTTACGGGACGAACAAGGTATGCTCTCAGATATGGGGATACTGTATTTCCTCTGTCTCTTCATTGAGAAGCAG |
| | | TACGTTAGTATGCTGTTCGATCAGTTGGGTTACAAAGGTTATGCCATCTATGAGAACCATTCAGAACTGCGTAGA |
| | | AAGTACATACGTGAGATTTTCAGCGTCTATCGCATTAAACTGCCGCGCGAACGTATTGTTTCTGAAAGCGCTATC |
| | | GAGGCACTGGGTATGGACATGCTGGGGGATCTGAAACGCTGTCCTGAGGTCTTATTCAAAACGATTAGTGCTAA |
| | | TGACCAGGCGAAGTTTAGAATTAAAGCCGAAACATCTGGTGAAGAAACCTTATTCATTCGAAAAGATTCTCGGT |
| | | TTGCACGATTGGCGTTGCAATACATCGACGAAAAGGAGCATTTTAAAAATATACGTTTTCATGTGTCATTGGGCG |
| | | AATATCGTTATAAGTTCTACGAAAAAAGATGTATCGATGGGGATACGCGCGTTCGGATCTTACAGAAAGACCTT |
| | | AATGGCTTTGGCCGCCTTGCCGAGATTGAAAAACTGCGTACCGAGCGGTGGGGGAAATATATTCGTCCTTTTGA |
| | | AAGCATTCGCATAGATGATGTTCATACCCGGCCATATATTACGGATCAACGTGCTAAGTATCTTTTTAACGGTAA |
| | | CCGAATAGGTCTGTGTTTTAACACAAAAGATGCACAGGTCATGTCTGCCGGAGTGTATATGCCAGCAATTGACG |
| | | GCGACAATGCACCTTGCGTCCAGCCGACCTGTTGGCTGAGTGTTTATGAGATTCCCGCCATGATTTTTCACTTGT |
| | | TACTGTGTAAGGAAGATGACGCATTGGAAAATACACGAGGCAGCGATACGGAGTCAATAATCAAAAACTACGT |
| | | GGCGAATTACAAACGTTTCTTTGACGATTGGGCGAAAGGACGGTGGCGTCGGAGCGGGGATGCGGCTAAAGAT |
| | | TTTGCTGACATAGAAACGGAGTATTCGATTAAACAACGTGATCTTCCGCAGAAAGTTTGTGATTATATTACTGGG |
| | | GTAAAAGGAAAACCACTGCAAGAAAAAGCTTCCGCGATCGTCAAAGCGATGCGGGCCGAAAACGAAGAAATG |
| | | ATCCGTCGTTTTGATGAACGCTACGCACGTGCAATTAGCCCGGATAATAAGTTTGGAAAACGTTCTTTTATTGAA |
| | | ATCCAACCTGGACGTCTGGCTGCCTTTTTAGCCGAAGATATAGTTCGCTTTACCCCGTCAGTTGATGGTAAAGAT |
| | | AAACCCACGGGCAAAAATTTTTCGGTTTTACAGTCAGCTATAGCAATCTACTCGTTGCGGAAAGGATATGATAT |
| | | GTTGGATGAGCTGCGTCGAATCTTTCTCTCCGCTGGGATTATTGGACAGAGACAGGGTAAAAACCATCCTTTTCT |
| | | GGACAAGGTGTTAGCGGATGAACCTCAAGACACCATGCAACTGTATCGTGCTTATCTTGTGCAACGTGATGTGT |
| | | ATCTTAGACGTGTTAACGCCACCAAAACCTATACTTCAGCGTATTTTATCCATACAACTCGCGCTAAGTGGGCTA |
| | | AACGCACAGACGAATATTACAACTCTCTGGCAAAGAGATATCTGAGCCAGCCGATAGAACTGCCACGCCGCCTG |
| | | TTTGAAGACGATATTAAACGTCGATTAATCAAGCGTCATCCGGATATGGCAGCATGCTTGCAGCAAGAACGCTG |
| | | CAATGTAGCCTATATGATTAACTGCTATATGCAGCAGGTAGAGCACGACGGCGTTCAGTCTTTTTATGCGATGCC |
| | | TCGTCGCTATAGCTGCTTTGCAGATGATACGTATCGAACTGTCGATGAGATGCAGAGCTACTCCCATATAGACCT |
| | | TCTGTCTCATTATCTTCGCGAGTGTAAAAATAACGCGTCGGAAGAGGCCCCGTACATCGAAGCCGACGCACGTC |
| | | GCCGCTTTCACGCACTGTACAAAAAATATGATGATAATGAACGTGTCATTCGCCGCTATATGGTTCAGGATATTT |
| | | TACTCTTCCTTATGGCCAGAGACTTGCTGATTAAAATAAAGACCGATAAGAAGAGTTTTGAGGAATACAGACTC |
| | | CAGAAAGTTACCCCAGAAGGTGAGGGTATACTGGAATATAAAGTACCGGATTTTGCAGTGACATTAACTTTCGC |
| | | CAACGGCGAGAAACGTACCATCCGCCAACACAATCTCAAAGTAAAAAATTATGGAGATTTCTTCAAGTTCATGT |
| | | ATGATGAACGAGTGAAAACTCTGCTGCCACAGGTTAAAAGTTTGGAAATCGATCGGGAAGTGCTGGAACAAGA |
| | | ACTGCAACACTACGACTCCTCTCGGCCTGACGTCTTCCGCTTAATATTAAAACTTGAGCAGTTCATTGTTGAGCA |
| | | CTTCTCCGAACTCAAACAGCAACGTGTGCAGTTTCGTGACATTTTAGATAAAGTAACTTTAAACACCGCGGACA |
| | | AGGAAGTGCTCCGCCTTTTACGTAATGCCTTTAGTCATAATAAGTACCCACGCCTGAGTCAGATTGAAGAATATG |
| | | CCCTGCCGGGCGTAGCAGTTTCGTTACAGCATATTGCAGAGGAATTAGTAACTAAAGCCGGCAGATAA |
| 356 | JW13 | ATGGCTCATAGACAGCAATGGAATAAAAATAAAGCGTCTGATGGTAAAAAACCGCGAAAAGCTAGCTGGCAGT |
| | 181 | GGAAGCAGTCCATGACGACTGAAATGAAGCCCGTGATGGCGTCTTACCTTAATATGGCCCGCCATAATGTGTAT |
| | | CGTACTTTAATTCATATTTCTACCCAAATGCAAATTGCAGAGCAAGGTGATGAAGATAAACTTGCAAAGTTCGA |
| | | GATATGGAAAAAACTGGAGAAAGCCCCGGCCCCTGAACAGATGAAATTGATAAAGCTGTTGCAGAAACACTTT |
| | | CCTGTTCTCCAAGCATTTTTTAACTACGAGAAGAAAGCCGGCAAGGTAATGGTGGATACTAGCGCGCATGATGT |
| | | CCGTCATTTCTTCGTCAAACAGCTGCTCCCGACACTGTCGCGCATACGTAATGAATATACCCATTACGCAGCGAA |
| | | ACCTCGAACACAAAAAGAGGAAGGTGAATTACGACCATACCTTCGGCAAGCACTGGCCGATAGTGTTCCCGCCG |
| | | TGTTCGATCGGTTTGCGGTTGAAGTCGTAGAAGATAACAAGTGTAAAAAAAAGAAACCGACTCAGCCAAAAGT |
| | | AGTTTCGGAAGCGGTTAAAAAAATTTTTCCAGAACCTTTCGACAAAAAGGGTAATAAAAAGAGTATCTATAACC |
| | | TTGTATCTGGCGACGCCTTTAGTGATCTCGGAATCGTCTTCTTTACATGCTTATTTCTGGAACGGCGTTATACTGC |
| | | GATGTTCCTGGATGCATTGAAACCTTGGCCAGAGTCCTTTAATGAGGTGCAGCGTAAAGCAGTGTTTGAAGTTTT |
| | | TACGTACGATCATATTCGGTTACCGCGTGAGAAGTATGATTCTGTAACCCCGGACTATGCTCTCGGTCTGGAAAT |
| | | GCTGAACGAACTGCAAAAATGTCCTGCTGAACTGTTTGATATCCTGTCCTCAAAAGATCAGGAACGGCTGTCAG |
| | | TGGATATTAAGAGTGATGATCCGGACGTAGTGACGGACGATGGAGTTACCGTCAAAGGCGGAAAAGTTCAGAT |
| | | GAAACGTGTCTCCGACCGCTTCGCACGTATGGCAATGATGTATATTGACCGCACTAATGCATTCAAAAATCTTCG |
| | | TTTTATGGTGCATCTGGGCAATTATCGTTTTAAGTTTTACATGAAACAGTGCGTTGCTGATAAGGAAGCTGATAC |
| | | ACTGCGAACTCTTCAGAAGGAAATCAATGGTTTTGGACGAATAGACGAAATTGAAGCCGCCCGTAGACAGATGT |
| | | ATGCGCCAATGTTTAAAGCGACGGTTAAGAAGGAGAACGCGGCCGGGGTCGAGGTTGAAGAATTACAACCGGA |
| | | TCATGCGGGCATGAAACCCTATATGACGGACACAAAAGCTAACTATGTCTTTGATAATAACCGCATTGGTCTGC |
| | | GTGCTATTGGCGGGGATCGCGAACCACGCCTGTATCTGCCGTCTTTGACGGAAAATGGTGAGCCCATTGATGAT |
| | | GCAAAACAGGTTAAACTTTACACCCCAAATGCTTGGTTGAGCGTATACGAGTTGCCGGGCCTCATATTCTACAA |
| | | ATTATTATGTGACACATATCCCGAGCAAGCAGAGAAGTTAGGCGATGCAGAGGAAGTCATTAAAAGCTACATA |
| | | AACGCTTACAGAAAACTGTTTAAGAATATCAGAGATGACAAGTTCGACGGATGGGATGAGAAACGCTATTCACC |
| | | ACTGACGTTGGAAGATCTTCCCGTCAAAATCAAAACGTATATTCTGAAACCTGAAGCCTCGGTCAATCCGCTCTT |
| | | TGCAAAGAAAGCACAGATAGTGATTAAGGAAATGGTGGAAGCGACAGAAAAAGAGATTGAACGGTTCAATCGC |
| | | AAACTTGATCGGTTAACAGACCGGGAAGTTCGCCCGGATAAAAAAACAGACCTTCGTCCGGGGGCTATTGCAAC |
| | | TCGACTTAGCCGCGATATAATGTATTTTGCACCTAAGAGTGAAGCAAAAGATAAGATGACAAGCGCGAATTTCA |
| | | ATAGTCTGCAGTCCGCGCTGGCGTTGTCGAAGTATTCCGTAAAACGAATGCGTGGTATGGTGGATGAACTCGGG |
| | | CACCCGTTTATTGGCGAAGCATTTGACAATTATGCCGCTACCAACTTTAAGATCTATGATTTTTACAAAATCTAC |
| | | CTTGAGTCCCGGTTAAAGTATTTGAAGTCAAAACAGGGTCTGGGTAAAGCTGAATTGGAGAAGTTACCTTTCCT |
| | | GCACCCGGACCGTGCCAAGTGGCAGAAACGCGATAAAAAAGGAATCCAGGAACTCGCGGGGCGTTATCTTAAA |
| | | ATTGGGGAAGTTGATTTTCCACATGGGTTTGAGTTACCGCGCGGATTGTTCACAGCCAGAGCTGAAGCTATTATG |
| | | AGAGAGGCAGGTGTGTTGACTGAACCGGCTGGTGATCGTAAACCGTCAATGACCAATCTTATCAATCATTACTT |
| | | TGAACACGTTTTAGAAGACGATAACCAGCCCTTTTATTGCTGGTCTAAACACTATGAAGTTTTTGACAGACTCGC |
| | | CGCTAAAAAAGATGACAAACATCCTACCCCACAGTTCTTAAGCCCAACTCAGTTGTGTGAAAAAATGAAAGTTC |
| | | GCAAACGCCTTAGACCATCAAAGTTCATGTTAAACAAGGCCCTGGAATCGGTCAATAACGGCCTGCCTGATGAG |
| | | GAGCATATCAAGGATATCTATGACTATCGTATTACCTCACTGGCCAAAGAACGTCTGAAAAAAGACTATGCCGA |
| | | GTATGATGATAATGAACGCATGCTGCGGAGATTTGCGGTGCAGGATAAACTGATATTTCTGATGGCCAAGGATA |
| | | TCTTGCTGGGTGTTGGGGGAATTAAAAAAGAAGCACTGGGTAAGTTTAAACTGAAAGATGTTATGCCGGGCGCA |
| | | AAAGATAGCATTCTGGAAACCACAGTGCCCTTCATTATCACCTTAACGGTAGGAGACACTATTTTAACCATCCGC |
| | | CAGAAAGAGGAGATTAAAATTAAGCGTTATGGCGAATTCTACCGCTATAACAGTGAACCGCGGCTCAAATCTTT |
| | | ATTACCTTACCTGACTAAAAAAATTGGCGGAAACGCTGATGCCAAACTGGAAATCGACCGTGAGGAGTTAGAA |
| | | GATGAACTGAGCCGTTATGACCTTAAACGGGTGGACGTAATGTCAATGGTACAGACCTTTGAAAAAGGTATTAT |
| | | CAAACAAGCGGGCAAGGCCGGTATAAGAATCGACGATGACACGCTGATCAACTTTAACAACCTGATAACCAAA |
| | | ATTGGGCACGTGCCGTATGAGGGCCAAGGGAAAATCTTGATCAACATACGTAATGGTTTCTGCCACAATGAGTA |
| | | CGCGAAAGACATATCTATACCTGAAGAGACGTCACTCCCCGAAATTGCGGAACAAATTAGTACGCTTTTTGATA |
| | | CCACCCGCAAACGTCCAACCACAAAAGAGTAA |
| 357 | JW13182 | ATGGAAACCACGATTAATAACCGAATCGGAAAAGGTGAATACTATACCGAAGAAAGTAAAGAGTTCTTAGCCG |
| | | CCTACTTCAATCAGGCCATTCACAACGTGTTTATTGTTCTGAATCACATTGCAAAAAGATTTGGCATGGACGAGT |
| | | TATCAAGCGATGAGGAGCTGAAAAACTGGCTGATTGGCAGACAGCGGGAAAAAAAACGTAATGCGATCGATAG |
| | | ACAGCGTTTTCTTGAATTAATCGATCGCCATTTTCCTTTTTTGCGAATTGCGAATGCCGATAAAAAAGATGCAAA |
| | | ACGCGAGAATGATCTTGAAGACAATCTGGCGCTGCTCATAACCTTATTGAACGACCTGACGGAACGTCGTAATA |
| | | AGTACTCACACGCCATAACTCATGCGAGTATTGAAAGTAATGACCGCGAGTTAGTTTGGCGCTTATATTCAATCT |
| | | ACGATGCCAATATTAATTTGGTTAAGCGCGACTATTTTGAGTCAAACGTGCATACGGAAATTAATGAAGATCCG |
| | | TACGAAAAACAAGTCGAGCACCTGCGTCGTTTCTGTATGAATAACGATCGTACAAAAGTCGATGAAAAGGGAA |
| | | AGAAAAAGCCTGCGATGCCGAATCCACGTTTTCGTACGCCCTTTCTTAACGCAGAAACTAACCAGCTGAGTATC |
| | | TACGGCCTGGTGTTCTTCGTATCGCTCTTTCTGGAAAAGAAGTACGCCATCCAGATGCAGAAAAATGTGTATGGC |
| | | CTGAAAGATGCTCGCGACACGAAGTTTAAAATGACCAATGAAGTATTTTGTCGTTCGCGTATCATCATGCCGCG |
| | | AGTGCGCCTGCACTCAGACAAAAGTACCGACGCTCTGGTGCTGGACATGCTGAACGAACTGGCCAAAGCACCA |
| | | GAAGTACTGTTTGATCAATTGACTGATCCGTTCAAGGAGAAGTTCTACATAGAAAGCATCGATTCCTTAGAGGA |
| | | GAGCGACATTATCACCCCGGTGCGCGCTATTCGGAAACAAAACCGGTTTATGTATTTTGCTTTACGATATCTCGA |
| | | CGAGTCTAATGCCTTCAGCAAACTTAGATTCCAGATTGATCTGGGTAATTATCATTACCATCTTTATGAGAGCAA |
| | | AATTAACGATCAGACAGAAAGCAGACACCTTACAAGAAAACTTTTTGGGTTTGGTAAACTTATTGCCTTTGAAC |
| | | AAGAATTTGCGCCCGAGGAATGGAAAATGAAATCAAAAGATCTGGACTATTATGAAGGCGCCACCCAGCCGTTT |
| | | ATTGCTAAAACCTATCCCCACTATCATTTGGAAGAAAACAAGATTGGCATTTTGTTCAATGGACAGGCCGAAGT |
| | | TCAATGGCCGCATCTTGATGTTGAAGAACACGAAAGCTTTCCTAAGTATAAACGACGCGCAAATGAGAAAGCAG |
| | | ATGCGTTTTTAAGTGGGAACGAACTGCTGGCTGCGGCGTTTTGTCATCATTTGTATGCGTCGATTGGTAAACCTA |
| | | ATACCGTGGAAAAAATCATTCGCGATAAGTACCATGCTTTACGGCAGCTGTTTTCAGATTTGAAATCTGGCAATC |
| | | TTCAGAATCTGTTAGGTGACAATACCTCTAATGAGGCGATTTCCCAGCTCCTGTTTGAAAAGTACCGTTTGACAT |
| | | TATCGGAAGTGCCAGTACGCCTGCATGCATTCTTGAGCGGTCAGGAGCAGGCTGATACTAAGGCGATAGCGCGC |
| | | GGGAAACTTGAACTTATGGCCCAGCAGAACAAAAAACGTATAGAGCGGTTCGACGCAATGAAAAAAGCAGTGG |
| | | TTAAAGTCGGAAAGGCTCAGTATCGCACGTTACGTTCTGGTGATATAGGCGATTGGCTGGTCCGCGACTTCATGC |
| | | GGTTCCAGCCGATTGGGTATAAGCGTAACCAAGCTGGCAAACAAGAACCGGATCTGAAAAGCAAAGCGAATCC |
| | | AAAAAAGTATCAATTAATCCAGAAAAGCCTGGCTTTGTATGAGCAAGAAAAAAACAACTTGTTAGGGCTGTTCA |
| | | AATCATGCAATCTTCTGTCTTCGGAGAATGAACATCCGTTTTTAAACGAGGTTGTTCAAAGCATGCCTGCAACTT |
| | | GGCAGGACTTTTATGAGCGTTATCTGCATGCGCGGTCTCGGTTCCTGGAAAAATGTATCGAGAAAGGTATTAAA |
| | | AAGAATTCCTATCAAGCCTGCTATTCTTTTTTGAAGATCAAACCCCAGCTCAAAGATAAGGAAAAATTGTATCA |
| | | AGGATGGGACGCCCAGATGAACCTGCCAACAAACATGTTCATCGATGCGATCCATGATTGGTTTCGTCAAACAC |
| | | ATCATGAATCCTTACGCACATGGTTTACACAGCAGGAAAAACCCCACCAATTGATAGGCCTGATTAGAAAGTAT |
| | | ATCGAACTGGCTCATACAGATCAAATTCAGGGATTCTATGATATGTTTCCTTTACGTTACGACTTTTACAAAAAG |
| | | GAATTTCCGAACGGTTTAGTACTCCACGAGCGCATTAATAAACAGAAACAGATTTGGGAAGCCCAGTTAATTCA |
| | | GACCAAAAAAAGACTCGAGGATGCGGCTTCCAAACTTAAAAAAGTTAAACAGCAGGTCGAGAGCTTGCCGGAT |
| | | CAGGAACTCAGATTCCGCAATGAAACAGAGGCCATGGTTTATTTTACAAAACTGTTTGACTCCTCTATTGTAAAG |
| | | AATGCCATCTTAAAACTTCAGCGTGACCAGAAAGCTTTACGGGTAAACACCATTGGCGAAAAGATTATCAAAAT |
| | | CTACACAGCAAAGTATGATGCAATCCATCAAGAGGTGAGAGACTTTAAAAGTATGTTAACGACTGAAAAAATG |
| | | ATACGGCGAGTTAAAGCGGAAGATTGCGTCACCCTCTTCATGTTAACTGATCTTATGAATCAGTCTCAGATTACC |
| | | ATTGGACAGGAACAACGTACTATTAAACTGTCTGATATTCAGCCAATGGGTGAAACTCAGATTCAAGGTATTTT |
| | | AGATGCAGTCCAGGTACTCGAACAAAAGTTGGATTTTTTTTCCTCAGATGAACAGGGGAAAATCTCCCAAGTAA |
| | | AACTCGGTGAGTGGACGATCTTCTCAACGGATACGAAAGTTAAGAAGCAGGGTAACTTTAAACAACTCCTCAAA |
| | | GATCGACGTTTAAACAATCTGGCACACTATATTCTGCCAGATATAGCCGGTGGGGCTATACGGGTTCGCCGTAA |
| | | TCTGCTGGAAATGGAGCTGGATCAATATGACCGCAATCGTATACCCATCATAAAACTTATGTATGAGCTTGAGT |
| | | ACGCAATATACAAAGTGGACCCATTCTCTGTTGAGTTAAAATATAAGAAATTTTCACAATGTTTACGCGAATAC |
| | | GCGCAGAAGGCAGTGCTGAACGTTGAACAGGTCGAGCATCTGAATGTTTTAATCGCAATTCGGAACGCTATTAT |
| | | GCACAACCAATATCCGAATCGTGATCATCTTAAAGCAATAGTCCCTTTTACTGTAAGTGAATATGCACCTGTCCA |
| | | AGAAGGCCTGACCATAGCTCGTCAGCTGCTGGCCTGCGCACAGGTGTCGGTGAATATAATCCTGAGTACTATAA |
| | | GTAAGTTCGAATAA |
| 358 | JW13183 | ATGAATGAAATTAAACAGCCGAATAATTCAAAGTATGTTTATGCACCGTACCTTACTATGGCTGCCTGTAATATA |
| | | GATGCTGTCTTTTATCACTTGGAAGATATTTTCAACCATAAACAGAACAACGCGATAGAGATTGATGATGGCAT |
| | | AACACAGCTTTACAAATTAAAAATATTCCAGGATCTGGGTATCTCTAATGGAAGCAACGATGAGAATACTCCTG |
| | | TTGATTTTCTGAGAAAAGATGCAATTATCAAACAGATTTGTCGTAACATCCCGTTTTTTAACATCCTGTTCGACTC |
| | | CAAAATCTTTAGCAACAAAAGTTTAGATGAACGCTGTCGCTGCGTAGATCGATATATTCGCGCCCTGTTTGACAT |
| | | GCGCAATTATTATAGCCATGTGCTGCACGAAGAGATCGATATTACTCATCAAATCCCCAACATAGAAAACGACT |
| | | TGAATTACATTTTTGAAGAGAATCGTAATACGGTTAAAAAGCGGTTTGTCTTAAACGACGATGATATTAAACAC |
| | | CTTAAAGTGTTTCATAAAGACAAAAAAGGGAGTAATTCGTACTATAAGTTTCTGGAATCCGGAGACCATTGTGA |
| | | TAAACTCACCGTCAATGGGCTGCTGTTTTTCACGTGCATGTTCCTGGAAAAAAGATACGCGTACGAATTGCTCAA |
| | | GAAACACTACGGGTTTAAAAACTCATCTAGTAAAATGCATAAAGCGACCTTGGAGACCTTTACAGTGACGTGTA |
| | | TTCGCCTGCCTCATAATAAACTTGAAGTTTCGGATTCAAAAGTGACGCTGGGTCTGGATATTGCCAACGAAATTT |
| | | GCAAATGCCCTGACGAAATTTTTTCACTGTTGAGTATTAAAGATCGCAATAAGTTCCGTATTCCTATTGATAAAA |
| | | CTGATCCCGAACAAAATATTAACGAAGATTGCGAAGACAATGATACCCAAACGATTCTGCAAAAACGCTATTCT |
| | | AATCGTTTTCCTTATTTTGCCCTGCGTTATCTTGAATTCATGAATCATCTGCCGGACATCTATTTCCACATTGATA |
| | | TGGGTACATACTTTTATGACTGTTACAAAAAACAGCTTATTGATAACAGTGAAATCAAAGATCGAAGATTATCA |
| | | AAAAACATCCTGTGTTTCGATAAAATGGACAATGCGGTGAAAACCTTTAATGATTCAAAGAATTCAGGCTTATA |
| | | TGCCGCCAAGTACGATGAAAAGAACGAATATCGTAACAACACGTTCCCACACTATCATATCAACAACAATCAGA |
| | | TTGGGATATCCCTGGAAAAGTATATACCTGAACTGCGGGGAAAAGATACCCGCTTACGTAAACCAGATGCGTTA |
| | | TTAAGCATCTACGAGTTACCACTGATTGTCTACCTGTGCCTGAACAATAAAGGTGAGGATGTTACGGAATTGAT |
| | | AAAAAACCATATCAATACTTCTAAACTCTTCTTTGAGTATGTGGCATCGGGCGGCAGATATAGTATCGACAATG |
| | | AGAATATGATTATTTTTAATGCGGGGAATAAGATTAATTCTCATCATTTGCCGGAGTTATTTCGCAAAGATAAAC |
| | | TGAATTATAAAGATCTGGCAAGCAAAGCCCTCAAAAATCAACGTGAGAAAGCTCAGAATATAATAGACCGGTA |
| | | TTACACCACTAAGGATCGCAAAGGAAAGATACTGAAAAACAAACGTAGTTTAAAAACAGGCCATATCGCTGAT |
| | | ATTCTTATGGAAGATCTTATGCGCTTTCAGCCAAGCATCGAATATACAGCAGGAAAAGATAAAGTTTCATCCCC |
| | | AAATTATCAGGCACTGCAGAAAGCATTTGCATATATCGGTTCGCGTTATATGGAAATTCCTAATTTGCTGAGAAA |
| | | AGCACGTTTAACTCATTCTGAAAATAGCCACCCCTTCATAGATAAGTGCATCTCTCCAATGCCGAAGAACATCA |
| | | ACGGTTTCTTCAAGCGTTATTTGGAAGAGAAAATTAAGTACATTGATACAATCGTAGCAAAAGGTGACTTTACC |
| | | AATGCTTACTTTTTACGTGACTATATTAAAAAATATGGCGCTGATCTTGAAACCGAGCGTCGTAATGCGGCGCAT |
| | | AATCTTATAGATAAACCGGCCGATATCCCACGGGGCATTTTTAAAGATCTTTTTTCAACAGAATATGCTTCTCCC |
| | | ACCTATCACATCTACAAAGAATTACTCGAGAATGAGGACGATGTTCAGGATTTTTATCATTTGAAACTCAATTTT |
| | | CCGGAGTACATTAACGACAAGCAAAAGTATAAAGTTTATCAGAAGATTAACTTCATTCGAGCTTGTGACATTGT |
| | | AAGCTTTTACATGATGAAAGATCTGATCGGTATCGATAATAGCAGCGTGAAACTTATGAGCTCTGGCGGCGACA |
| | | AACATATATTAAACTTAAGTCAGAATGTCGATATTCCGGTAATGCTGCTGAGAACGATTGGTAAAGGTAAGAAC |
| | | ACAAAGACTAAAATTAATGAGCAAATCATCTCCGGAAAGATTAAAATAAAGGACTATGGCAAATTCCGACGTTT |
| | | GATATTCGACAAACGGCTCTTTTCATACTCGCTGTATTTTAATAAAAACGAAGAAGGGTCGGAAAAGGATAAGT |
| | | TGATATTTGACGACATTGAGTTTGAATTCACCGAGTATGACAAATCGCGGCTGAAGATATTCGAACTGATCCAC |
| | | AAATTTGAGGAACGGGTTATAATTAAAAATGCCATTGCCTTACCGGTGCCCAGCAATTCCGACGATAAAAAAAA |
| | | CAAGAAATACAGTTACATCAGTTTCCCGGATATCCTGAAAAAATCCGGTCTTAATAAAGAAAATATGGAGCTCC |
| | | TCAAAAATATTCGCAACGCGTTTTCTCATCACGAATATTATGTCGTAAAAGACCTTAACGCGAACAAGCGAGAG |
| | | ATCACCAAGAAGTTATATGACAAGTGTGAAAAGGATATTATTTGCATAACAAATTCCATATAA |
| 359 | JW13184 | ATGCTGATCATAAAACCGTACGGACGCAGCCAGACCCAACATGCTTCGGACTCACAGCGCAATCGCCTGCATCT |
| | | TCCATCAGGGCAGGCCCATGCTGAGGCGGTCGAACAGTTGCAGAATAACCTTGACTTTGTGATGGCTCAGTGGA |
| | | TTAGCGTTATTGATAAAATTATACGCAAACAACCACAGCGTAGCGGAAAGGGCGACAAAAAAATCGCAGCCCC |
| | | ACAGGTTTCTACAACCCAGTTCAATGCCCGCAAAGCCTTAGGCATGGCTGCCCTGCAGTGCATCGAAAAAGAAT |
| | | TTTCTGAGTTAAGCGACCTTCAAAAGAAGCAATGGGGTTGGAAAGTCCATCCCTACGATTGTACTAATCTTGTTG |
| | | AGCCTCAACCGAAAGATCGCATTACTGGCCGTTGGTACAGCCGTTTTGTGGGAGCCATTCATCCGCAAGATATC |
| | | ACGCAACAAACCGTTCCGGCAATTGTGCAGAAAATCCATGATCATCTGTATCAGCAGGCGCTGCGTCCAAATGC |
| | | ATCCACGGCCGTGCATACCCAGGGTCACACTACCCATCAGCTCCATACAATAGCTAGAAACGTGCATGCTTTAA |
| | | AAACGCCGAAAGCGTCTACCCACGTGAGTTCAGATTCGGCATGGCAGAGTTATTTCCTGTCTAATGATGTAGTG |
| | | GCCTGCATCTACAAAGAGCTTCAGGACAAGCATGATCAGAAGGACAAGACGAAACATCAGAGCCAGCCTAAAC |
| | | CGCTGATTCCGACAACGACTCCAAAAGCCCCGTTATTTTCCGACCGGGCGACTATTGCCCAGCATCTGTTTAACC |
| | | ACTATGCCAAAGTGTTCGCAACACCTAGTGGGACCCCTACTCCAGTGAAAGATCTTTTAAACGAAGCGCATCCA |
| | | TTGCATGCACAATGGCTGGTGCATCAGTGCATTAAAGCCACCTATAAGGCATTGTTTGATAATGCGTCAATCAA |
| | | AGGCCTGAAGCACATACAACATAAGCTCCCAGCCAATTCTGAAGCCATGAGACAGCTTATTGAAAACCGTCGTA |
| | | AAAATCATGATATTGGACACCTGGTACGACTCGGGAAAGTACTGCATTATGAAGCTGCCGAACGTGGACAACTC |
| | | CCGCGGGCTTGCTGGGGCGAGATTTCTACACCTGCTGTCGCAGAGGGTTCAGCATATTGGCTGTCAGAGGGCCA |
| | | AGCAGAAATCAAACGAAGCGAAGCTTTGGTGCGCAGCTTCAAACAGGCAATCACTCACGCTGGTCATACATTGT |
| | | CCCAGCTGTCAGATTGGTCCGGTAACGGCGATATTTTTCTGAACGCTTCCGAAGCTGCGAAGCAGGCAGATGCA |
| | | AAAACCGTCGAACAGCGTGTAGCCTTATTATTTGGGAGAGCATGTTCAGACCTTTTTCCCTCGTGTTATGCGCCG |
| | | TCTGGGAATCTGCAGGCCGCAATTCAAGCCCTGCTGGCGCAGAGCATCGAAGCCTGGGCTCAGTTACGTCATGC |
| | | CAGTTTTCACTTCAAAGGTAAACACGTGTTCCTCGAAAAACTGCAGAATTCCTTGGCGAAGAACAATGTATCTA |
| | | CCCCGGAATTAGATGCGGTGGCCCAGAAACTGTGGGAACAAGACGGAGAACAGAGACAGAAACGCATACGAG |
| | | CAAGTTTAGAGGCTGCGCATTGTCTGGATCATCTGAGCCAGGCTCAGCTGAACGCAGTTTATCTTTGCATCGCAG |
| | | GCGATAAGCAAACTAGAATCGGTAGTTCGTTAGATATGCCCCGGTTGAACCGCGTTTTAAACCGGGCGCAAAAT |
| | | ACAAAACTTGGTATGAAACCGCTCACCGACGGTGCCCCGACATTACAGTTATCAACGCCTGCTAAGCGCCAAAC |
| | | CCTGGAAAACTCTCCCGCCCAGCGATGTCGGTATGTGCTGCTGAAATTACTGTATGAGCAGGACTTCGCGGCTTG |
| | | GCTCCAGGATACACGCCAGGAAACAATTGATAAGTGGATAGGTGGAGCGATCGCGCGTAACACAAAGGCAGCC |
| | | AGAGAACTGAACGGAAAAGGCTTGCAGCCTCAGACCACCATACACGCTAAGGCGGAACGGCTGTGGCATGAGC |
| | | AAGCGCAGGCACCGACAGCTAAAAGTACATCTCAGGCCAATCCAATTGCTCGGTTTTTTCACGATCTGGCCGCA |
| | | GCAACGGCATCAGAGTTTCGTGTCCAGCGACACTATGCCAGTGACGGTGAACAAGCTCAAGCGCAAGCGGCTTA |
| | | TATTGATGATTTGAAATGCGACGTAGTCGCGCGCGCATTCTTGACGTATCTTAAAAATAAGTGGCCATGGTTGTT |
| | | ACAGGATCTTGCGGTTCAAAATACGCCCTCTTGCATGGATGCACTGCCCGCGGATGCCCATGAAGCCTTGCAGC |
| | | TGGACAATCTTTGGCAACAACGTTTATACTTTTTGTTACACATGGTTCCGGTCGGTGTGGTCTCCCAGCTGTTACA |
| | | TCAACTCCGTAAGTGGCAGGTTCTCTCCACTCAGGCGGAAGCGTCGAAAACTGCCGTTGAAGACCAAGCACTGT |
| | | ACACACATTGTCTGCAGGTAATGGTGCTTTATCTGGATATGCATGATGCTAAGTTCGATGGCGGGACGGCAATA |
| | | CACCTGGACGATACTCATCGCCAGCCCTTAAAAGATTGCTTCAAACGCGTTGAGGATTTTGCCGCGTGTCTCCCT |
| | | GCCTCTGCAAATCCTTCACAACCGGGAGATGCGCACATACCGTTGCGTGGCCTGCGTGAAATGCTGCGCTTTGG |
| | | GGGTGCAAAAGTCTTAGCACCTGTATTTCGTCAACATCCAATTTCGGCGGCTGAATATGCAGACTGGCAAAAAT |
| | | TACAGGCTGTAATTAAAGAGGCCCAAAAAACACGTGAGGATCTTCATGCTGAGTGGGTAAAAAATCGGAAAGG |
| | | CTTTACCGGCCAGCAGGCTTACGCGCAGGCCCTCGAGACGTGTATGTCGCATAGACATCTTGCACATAGAGTCC |
| | | GGTTAGTTGACCATCTGCAACTGCATTCGCTCCTCATGGCCGTCTTGGCGCGGCTGGCAGATTACGCGGGACTGT |
| | | GGGAACGAGACCTTTACTTTGTATCCTTAGCTCTGTGTTATCACGAAGGTCTGGATCCGGCCCAAGTTTTCAATG |
| | | AGAGCGGGAATCACGGTAACTATTTTGATGGTGGTCAGATCGTTGAAGCCTTACGCAACGAATCTAGCCCCAGT |
| | | AAGAGTATTCAAACTCAAGTTGCTATGCTGTTCGGTACTGATTGCAAAAAAGGGGATCCGATGGTCAATATTCG |
| | | TAATGCACTGTCACATTTTAACATGCTGCGTCATACCAAACACCAGACTGCACCTGCGCCAATTGATCTTACGCA |
| | | CTGGGTGAATCAGACCCGCAGACTGTTGGCACACGACCGTAAACTGAAAAACGCAGTTAGTAAGAGCATCATG |
| | | GAGCTTCTGGAGCGTGAAGGCTTAGTTTTGACCTGGCAAACCAATCACCAGCACCAGTTAGAATTGAGCAAGGT |
| | | TGACAGTAAAAAAATTGCGCACCTTGGGGCTAAACAGGGCACGGAACCTCACCATTCCGGCGCGTATTGTGCGA |
| | | TGGTTCGTCAGATATTTTAA |
| 360 | JW13185 | ATGTTAGTGATTAAGCCCTATGGTAGAACCACTACTACGGTTGAATCCAATGCACAGCGTAAACTGAATCGTAA |
| | | ACAAACCCCGTGGCGGGCAGACGACGTTGGCACTTTGAACCAGGAGCATGATTTCCTGATAGCTCACTGGATTT |
| | | CTATGATAGACAAGATTATTTGCAAACCTAAGGCCATCAAAGCAGCAGAGGGCCAAGAACGCAAGCGCTCGCA |
| | | GCCGGGAAAGGCGACAACGCTGCAGAGAGACGTTCGGCAGTTAATAGGTGACGCCGCGCTGGATTTTTTTAATC |
| | | CGAAACCAAATAGAGAAATCTGGCAGTGGAAGCTCCATCCCTATGAGGTTGGTGATGCTCCCACAGGCCAGGCC |
| | | GCTCCTAATATTAAAGGCCGCTTTTACAAAGCTTTTGTCGGCCATGGTGTGGAACCGCACCAAATCGATGCAAC |
| | | GCAGGCGAAAGCAATCACCGAAAAAATACGTGCTCACCTTATGACAGCGGCCTTAAAGCAGCAAGGCAATAAT |
| | | CCCCAGTCAGCGCAAAGCGAGTCGCGTTTAGGTCGTATTGCTCACCAGCTGGGAACCATCACTCAGAACGTTCA |
| | | CCATAAAAAAGATCTTGAACCACATGCGTTGGACGAGGCCGCTTGGCAGACGTATTTTCAGAAAGGGGATGTCA |
| | | TAGAACAAATAGCTAAAGCTCTTGCCGCTAAGTATAATAAAAGTGATAAAAGTAAACAGCCCGCACAGCCAAC |
| | | CCTGACGGATCGAGCTACAATTGCGAAGTTTCTGTTTGAGCATTACGCATTGGTGTTTGGGAAGACTACCCAAGT |
| | | TCGCGATCTCGTTAGAGAAGAACACCCACTCCATAAACAGTGGCAGGTCCACCAGCGTATCAAAGCCACATACA |
| | | AGGCCATGTTTGACAATGCCAGTATGAAAGGGCTGAAAGGGATCCATAAACGTGTTCCGAAAGACAAAAAGGA |
| | | GATGTGGGGCCGCATCCATCAGCGTGCCAATAATCAAAGCGTAGCCCACCTGGTGCGGTTAGGCAAAGTTATTC |
| | | ACTACCAAGCAGCGGAAGAATGTAAAACTGTGCGTGCCGCATGGCATACTTTACAGACAACTCTGGACGCATCC |
| | | CCGTATTGGTTAAGCGACGGCCAAGCCGAGATCAAACGTTCAGAAGCGCTGGTTAGAGTTTTTAAACTTGCGGT |
| | | AAGTCATGCGGGTCGATCCCTGTCGGACCTTACCCAATGGGAAGGTGGAGATATTTTCGATAACGAAGACAAAG |
| | | CCTCAAAAGGAGCCGTCGCGCAGGAATTCGGTGAACGGATGAAAACAATTTTTGGTAACGACAAAGAAAGCGT |
| | | CTTCCCATCTGTTTGGGCCACTGACGATGCATTGAGCGAAGAAGCCAGACAGTTGCTGGAACAAACTATTCAAG |
| | | CTTGGGCGCAGTTACGACATGCCAGCTTCCATTTTAAAGGGCGCGATGGGTTTTTGAACGAACTGAAACGCTCA |
| | | TTATCATCTGAACCGCATACGCTGGCGTACCCGGCGCTTGATCAGTTATCGAAAAAACTGTGGGATGAGGACAT |
| | | TATTACGCGGAAAGAACGCATTAAAAAAACGCTCGAAGCCGCACATTGTGAAACACATCTGGCCCAGCATCAG |
| | | CTGGAAAGTGTTTATAAGACGATTTGTGGAGAAAACGTGACCCGTTATGGATCCACCTTGGATCTTCCGCGTCTC |
| | | AACCGTGTACTGAACCGTGCTCGGAATGCGGACCTTAAAGTCGATACAACCAATGCTCAGGTCAGTCTCCCTAT |
| | | GCCTGCAAAAAACGAAGTACTGGAGAAATCCCCTTCTGCTCGTTGCCAGTACGTGCTCCTCAAAATGCTGTACG |
| | | AGCAAGGTTTCTCGTCTTGGATGCAAGAGAAAAATCTGTACGAGGTGGATAAGTGGATAGTGCGCGCAATTGAG |
| | | CGCTCCAACATCGCGGCTCACGCGATTAATGGATCAAACAAAAACAATAAGCAACCTATTGAAAGCATCCAAGC |
| | | ACGCGCCACGCGTCTGTGGCAGTCAATCGGTCAAAAAACTGGTCACGATTTTTCCAACGATCTGTTGGATGGAA |
| | | AGAGTCCCATTGCTTACTTTTTCCACCAGTTAGCGGCAGCTACTGCTACAGAATTTCGTGTGCAGCGATTTTATG |
| | | AATCAAACGGGGAAGCAGCGCAAGAACAAGCGGCGTTTATTGACGATTTGAAATGCGATGTTGTGGCGATAGC |
| | | ATTTGCTAAGTATCTGGGGTCTAAGTTCAGTTGGCTGTTACAGGGTTTAGACGGTACCCAGACCTGTAGCATCAA |
| | | CGATCTTCAAAAAGCGACTACCACCGCCTCGGTAGAAATATGGCAGCAGCGTCTGTATTATCTTATCCATTTTGT |
| | | ACCGGTAGGCGTTGTGTCACGATTACTGCATCAGCTGCGGAAGTGGCATATATTAACCGCGAAAGGAAAAGATC |
| | | GCGCACCTACACCGGATAATCTTTACGCGCAGTGTGCACAAGTGCTGGTACTGTATTTGGACATGCACGATGCT |
| | | AAGTTCGATGGGGGCACCGCACATCAGCTGGATGATGATCTTCTCAAGAAACTGAAAAGTTGCTTTGAATCCGA |
| | | AGCCGACTTTGCAACGCTCTTTCCGCAAGGTGCAGAGGCCGATTCTCGTATCCCACTGCGTTCATTACGTGAAAT |
| | | GCTGCGGTTCGGCGCTCTGGACACATTGGCGCCACTCTTTGAAAAACATCCTATTACACATGCTGAGTATGAGCG |
| | | ATGGCAGGCATTACAGGGCCAGATTGATGATGCGCAAAATCTTCGGGCCGAGCTGCATAGCCAGTGGGTAGAA |
| | | CACAAACGTAATTTCGATCAGGTACTGCAATATCAGGCCGCCTTAAAGATTTGCGAGGCACATCGGCATTTGGC |
| | | CCATCGCGTCCGTTTAATCGATCATGTTCAGTTACATAATCTTATGATGGCAGTGTTAGCACGCTTAGTTGATTAT |
| | | GCAGGTCTGTGGGAAAGAGATCTGTATTTCGTCACGTTGGCCTTGTGCCACCATCACCAGATCGACCTGAATAGT |
| | | CTGTTCGCCCACACAGATAAACATGGTAACTGCTTCTTTGATAACGGACAGATTGTGAAAGCATTGCGCAAGGC |
| | | TGAGTCTGATGAATGTTTTACGAAAGTACATACCACATTATCTGCGTTATTTGGAGAAACCTATCTGGGCGGCGA |
| | | GTTAAACGGCCTTCGCAACCGCTTCTCTCACTTTAATATGCTGCAGAAGAAAGAGAGCGCGTCAATTAATCTGA |
| | | CCAACGAAGTCAATGAGGCACGAGCCCTGATGGTCTATGATCGGAAACTCAAGAATTCTGTGGCTAAAAGTATC |
| | | TTAGAGTTAATAGAAAGAGAAGGGTTCAAGCTTAGCTGGAAAACAAATAATGAGCATCTTCTGGAAAGAGCGT |
| | | CCATACAAACTAAACGCATTACCCACCTTGGTAATAAAGCCGAGGAAGCTCATCATAGCTCGAATTATTGTACT |
| | | ATGGTTGCACACTTGTTTGGTACGACGTAA |
| 361 | JW13186 | ATGTTAATTATTAAACCTTATGGGAGAACACGTACAGAAGATAATGAGTCAAGCGGACGCCAGCGGAAACTTGT |
| | | AGGTAAACAGATCCAGTTACAAAGTGAGCCAGTTACTGTAATCGCCGAGAAGAATGAATTCCTGATGGCGCAAT |
| | | GGATATCAATCATCGACAAGATCATAAGTAAGAGTCCGGTCAAACGAATTATTGAAGGTAAGATCAAACCATGC |
| | | ATTGCACCTACCCAGCGACAGGCACGCGAACGGATTGGTCAGGCGGCAATCAAGTTTCAGCCCGCCTTAAAGGA |
| | | ACTGGAAGCCAATTGGAAAGCTAAAATCCATGCTTATAAGATAACTCGTACAGTTAGTAGCGGGGATCCCAAAG |
| | | ATCGCATTAAGGGACGATCCTATGCCAAGTTTGTGCCCCATGGAATCGAACCGGAGCAGATAACAACCCAAATT |
| | | GCAGAAGAGATCGCCAAGAAAATTCATGATCATCTTAATCACCACGCGCTGAACTCTAAGGGGACCGGGGGTA |
| | | AAGGAAAAGGATCCATCGCGCACCAACTGGGCAGCATAGCACAAAATGTCCACCAGGCGGATAAAGAAAGCGG |
| | | TCAGGAGCCGTCAGAGGTGGAATGGCAGAAATACTTCGATCATCACGATGTAGTTGCGGCAATTGCTAAAGCTA |
| | | TTCAGAGCAATAGACCGGCACGTACCAAACCTACGTTAACTCCGAAAGAAACTGCTAATACCGGCCAGTCCCCT |
| | | TCTGATTCTCCACGCCGCCTGACAGATACGGCAACGATTGCTAAACATCTTTTCGACCATTTTGGCCAAGTTTTT |
| | | GGTCAGAACGCAAAAGTTAAAGACCTGCTCAAACCGAGCCACCCGCAGCACGGGCTGTGGCAAGTCCATCATC |
| | | GCATCAAAGCAACTTACAAGGCTCTGTTTGACAACGCCTCTGTAAAGCGCCGAAAAAATATGGCGATACACATG |
| | | CCTTTAAACAAAGAAGCCATGATGGCGCTGGTGCGGCATCGCACATCAAATCAGGACCTTAACCAGTTAGTGCG |
| | | TCTCGGTAAAGTGATCCATTACGAAGCTGCGGCATGTGGTCAATCGGTCCGTTCGGCATGGTCTAATATTCGCCC |
| | | TAATATTGACAAAAGTCCTTATTGGCTGTCGGAAGGCCAAAGCGAAATTAAACGGAGTGAAGCCTTGGTTAGAG |
| | | TCTTTAAACTGAGCGTATCTCACGCCTCTAGAACCTTAACGGATCTTTGTCAATGGCAAGAGGGCGATATCTTTC |
| | | TGAACAGCGAGGATGCATCCAAAAAACTGATTCCCGATCGGTTTGTGCAACGTATGCAAACGTTGTTTGGACGA |
| | | GATCAAGACGCGAGTGATCCTGTGATACCATCTGCCTGGCATTCGAATGGGCAACTGCACGACGATGCACAAGC |
| | | CTTGTTAAAAGAGACTATTGATGCATGGGCGCAACTGCGTCACGCTTCGTTTCATTTTAAAGGACGCAACGGCTT |
| | | TTTAACGCAACTGACCCAGGGTTTGAGTCGGCATGCTGGCAGCTCCCCGCCGTCCAAAGTGGCGCTGGACACGA |
| | | GAGCGCGTAAACTTTGGGATCGTGACCATCTTGCGCGTGAGTCTCGTATCCGTTCAACGTTGGTTGGTGCGCACG |
| | | TGGAAACACATCTGAATCCCGTTCAGCTGGAGAAAATCTACACTTGCATTACGGCTAAAGACACCAATCGCTGT |
| | | GGAATTACACTCGATCTTCCTCGTCTGAACCGTGTCTTACAGCGGGCTCATAACACACTCATAGCGCAAGAAAA |
| | | TGATGATCAGATCTTAAAATTGCCACAGCCGGCGAAACGTGAAGCGCTGGAACAGTCTAATGCCGCCCGTTGCC |
| | | AATACATCCTGCTCAAGTTGGTGTATGAGCAGGGCTTCTCGGATTGGTTACACGCCAAAGGCATCAAAGAACTC |
| | | GACAAGTGGGTAAAACTTGCCATTAAACGCACCGATGAAAGTGCCCAGCGTCTGAACACCCCGTATTCGGATGA |
| | | CCTTCCAGTTAAGGCCCGTGCCAATGAGCTTTGGCAGCAGCATGTAAGAACACTGTCAACAAGCGATGCCCTGG |
| | | GCGGTATGTCAGCAATTGCTTATTTTTTTCATCAGCTGTCCGCCGCAACAGCTACCGAATTTCGCGTGCAACGCC |
| | | ATTATGAATCAGATGCGAGTGCCGCTCAGAAACAAGCAGCCTTTATTGAGAAACTGAAATGTGACGTAGTTGCA |
| | | ATTGCATTCAGCAAGTACCTTCAAAAAGATTTCCCTTGGCTGCTGTCAGATCTGGATCCGACGCAAAGTTGTTGT |
| | | GTAAGCGAACTGGCTGTGCATAATAATGCTACCCCAGTAGAACTGTGGCAGCAGCGTTTATATTTTCTGCTGCAT |
| | | TTCGTCCCAGTTGGAGCGGTGTCCCAGTTACAGCATCAATTACGAAAGTGGCACATTTTAAGTCACCATGGTGCC |
| | | GAACATGAATCTTCTTCTCTGGGTCAGCTGTATGCGTCTTGCGCTCAGGTTATGGTACTTTATTTGGACATGCATG |
| | | ATGCGAAGTTTGACGGTGGTATTGCATATCTCCTCTCAGATGAACAGCGTAAACAACTCGAGGATTGCTTCGAG |
| | | TTATCTGAGGACTTTCAGGCATGTTTCCCGAAAGGCTCAGTCCAAGACTCACGGATTCCGCTCCGCGCATTGAGA |
| | | GAAATGTTGCGTTTTGGGGGCATAGAACTCATGAACCCACTGTTCGTTCAGCACAAAATAACTCATACAGAGTT |
| | | TGACCGTTGGACTCAGTTGCAGTCGTGCATTGAGAAATTTCAGAAGTCTAGAGAAGACCTTCATCAAAAATGGA |
| | | TAGATCATCCTGGTGATTTTTCGAAACAGGATAAAGAGGCATACGGCCAGGCCTTAAAAATTTGTGAAGAACAT |
| | | CGACATCTTGCTCATCGCGTCCGGTTGGTTGATCACGTCCAGTTGCATCGGCTGATGATGACTGTTTTAGCTAGA |
| | | TTAACTGATTATGCTGGCTTATGGGAACGAGACCTGTATTTCGTTTGCCTGGCCCTGTGCTACAAGAAAAACATA |
| | | GACCTGACAACGTTGTTCAACCACGAGGACAAGGAAGGCCATAACTTTTTCGATGATGGTCGCATAGTGGAAGC |
| | | GTTACGTAAGGTGAAAAAAGATTTTCAGTTTCCCTTGTTTTCCGAAGATCCAAGCCTGCTGTTGATTTCCGGATC |
| | | AACCAAACTTGCGGGGGAGTTGGTTTTTATTCGCAATCATCTTTCACATTTCGGCATGCTCTCAGACCAGAGCAG |
| | | ATCAAAGAGTACCCAACTTCCCAATCTGACTGACGAAGTCAACAAAACCCGCCAGTTAATGGCGTATGATCGTA |
| | | AGCTGAAAAATGCAGTTTCCAAGAGTGTGATCGAAATCCTTCAGCGGGAAGGGCTGCGATTAGTGTGGCAGATG |
| | | CACGATCACACCTTACAACTCGATCCGAATGAGCCGATACAGACCCAGATGATTAAACATCTGGGTGATCCGTC |
| | | CAGCGAAAGTATGACTGAAAGTATGCACAGCAAAGCATATACCCAGATGGTTGATCAGTTATTCGGGAATAAAC |
| | | AGTTAAACTCGCAGACGTAA |
| 362 | JW13187 | ATGGACCGCAAGTATATGACCCTGGATGATAAGTACTATTTCGCAGCGTACATTAATATGGCCAGACATAATGT |
| | | CTACTTGTTACTGGCAGACATCAGCACTCGCATGGGAGAAAAAGTTCCCAACGACGATGCAAAACTGATATTCC |
| | | CGATATTACAAAAGCTGGAACCGAAGACTACCACAGGAAAAAATAGCGTATCGCCCGACGAGCAGTTAAAAAT |
| | | AATCAAAGAGCTGCACAAACATTTTCCTTTCCTCAAAACGATGCTGTATGCCTATAAGAAACCGATGGGTGCAG |
| | | CAACTGAGGAAGAAATACAGAAAATGGACTACGCTCCGGGCGATTATTATGATCTGTTTACCCTGTTCTTTAAA |
| | | GTGCTTAACGACTACAGAAACGAATTCACACATACGATTTCACAGCCCAATGTTAAAATACTGGGCACCGAGGA |
| | | GTTTCGTGTTAGTTATGTCTTTGATTTTCCTGCGAATTTGTTGTATGGTCTGCGGGTGACATTTGATACTGCAGTA |
| | | CGGAAAGCAAAAACCCGTTTTAATTTTACCCCAGATGATACTAAACACCTTCTGCGTCAGGGCAAAGGTGGCAA |
| | | AGAATTAGTGGGGTTTAAGTATAAGTTTCAGGCGGAAGGTTCCAAGAGTCTTACGATTCATGGTCTGACGTTTTT |
| | | CATTTGCCTGCTGCTGGAACGCAAGTACGCCACGTTGTTCACTCAGAAATTGCAAGGCTTAAAAGACAAACGTT |
| | | CTCGTGGGTTTAAAGCCACCTATGAAGTGTTTGCGGTGCATTGTATACGACTCCCAAATGCACGATATACGTCAG |
| | | ACACCAGTAACGGTGCCCTGTTATTAGATATGTTGAACGAGCTCAAAAAGTGCCCGACACTGCTGTTTCCGCAG |
| | | CTGCCGGCTAAAGGCGAACAGAATAAAGATCAAGATGCGTTTCGTATTAAAGTTGAAGATATGCCTGGCATGGA |
| | | AGAAGAGGGCGATTCTGGTTTTGTTCTCCTGAAGCGCCATGAAGATCGTTTTTCATATCTTGCTCTGCGTTTTATG |
| | | GATGAAGTTAAGTGGTTTAATCTTTTACACTTTCCGGTTGATCTTGGTAATTATCACTATCATCTTTACGATAAAA |
| | | TTGTGGATGGTCTGCCACGCATTGGTTCATTATGGGAAAAAATGATTGGTTATGGCCGTCTCCCTGATTATCAGC |
| | | ATAAGTTTCTGAACAAAGATTATCCAGCTGAGTGGCAACTCCTGTGGCAAAACCATGAATTAAAGAAAGAAAAC |
| | | GAGAATGACAAAAGTTCTTATATGCCGCCCACAATGCCTCACTACCATTTTCCTGATGGGAAAGCGGACAACAT |
| | | AGCAATCAAAATCACAAATCCGGAAACTTTGTTCTGGCCTGCGATACATATTGATGGGAATGATAAAAAAGCCG |
| | | GGAAACCAGCCAAAAAAGAACATCCAGACCTTTTATTAAGTAAGTACGAACTCTTTGCTTTTATTTTCTACGGAC |
| | | TTTTGATTGGGAAAGACCGCATCAAAAAGGAACTCGAGGATAAACTGGTTCAGTACTTCAACAAACGTAAGCAA |
| | | CTGTATCGCAATATTATCAACGGTAAACTTCTGCCATTAGCATTACCTGCTGATATTCCAAAACCTAAAGACAAA |
| | | GGTAAAAAACCGACGGGATATGATCAACGTAAAGAGTTGCTGAACCAGAAATTAGCCATCTATGGATGTACTGC |
| | | CGATGAATTACCAGACGAGATTAAGTGTTTTTTAATGGGTGTGATAACCGAATCATTCGAAATCAAGGCACGCT |
| | | ATCGGTTAGAAGATCTGATCGTAGATACCGAGATTCTCCAGGAGCGCATTAAGCGTCAGCTGAAGGAGAAGCG |
| | | AAAACCTGGAAGCAAAAAAGATGTCGAAATTAAAGCTGGCGTTTTAGCCGGCTTTCTGGCTAAAGATATCATGT |
| | | TGTTCCAGCCCGCTGATAAAACAGAGGGCGTCAAAGCTTCTGGAAAACCAACAAGCACTCTGTACAATGTCTTG |
| | | CAAGCTAAACTGGCGTTGTATGGTGCTAATGTCGATACCTTAGAAAGAACCTTCAATGAGTGTGGTCTGTTATCG |
| | | GGGTCCACGAAACATCCTTTTTTAGGTCAGGTCGTGAGCCGGAAACCGGCGACCATTGTGGATTTTTACATGGCC |
| | | TATCTGACCGCACGGCATAGCTACCTGTTAAAATGTGAGACCGACAAAAATTACCAAGCGTATCAATGGCTCAA |
| | | AAATAACCAGGCGCGCAAAAAAACTGAACCACACTTCGGTCAGCGCTTGGCTGCCGAAATGGAAAAACTGCCT |
| | | GTAGTGATACCACGTGGTTTCTTTGCAGAGTCGATTAAAAACGAACTGTTAGCGCTCAACAACGACAAACTGAA |
| | | TGCGGCAATAGTTTCTGCGCGCACACCCAATACATCCTTTATGTTTATGGCATATCTTAAAGAGTATCTTGATGA |
| | | CGATGTTCAAGATTTTTATGGGTGGCCGCGAGTTTATAAGATTTTCGACAACTTTCACGACAAACGTACCAAAAG |
| | | TGAGATGCGCAAAAGTCTGAAACCGGAACCCTTATCGGAAGAAGCGCGTAAAGGACAGATGGATGAACTTAAA |
| | | AAATGGATTAAGGAAACCCCGAATGACGAGGATTATGGAAAGTTTAAAAAGCGTTATGATGACTTCGAATCAA |
| | | ATGAGAAGCAGATACGACAACTGAAGGTACAGGATATTACAACGTTCGCCATGTGCGATTATTTGCTCAAGAAC |
| | | ATTAACAGAAACCTTATTAACTCCGATGGCCAATTTTATCTGAAAGATATCGCCCCGAAAGGCGACAAAGGCAT |
| | | TCTCGACAAACCCATGAGCGATTTTGGCAGAGTTTTTGATTATTGCTGCACATCTAAAGATTTTGCCGGAGTAGA |
| | | GATTACTATCGAAGGGAAAACACGGATTTTCCAGGACGACCTTAAAGTCAAAAATATTGGGGATTTCAATAAGT |
| | | TCATCAAAGACAGAAGACTGCCGAATCTTCTGGCGTATCTTCAGAAAGAACAGATTCATCGCGCAGAACTGGAA |
| | | AAAGAATTAGAACGGTACGACATTGTGCGGATCGAAATTTTCAAAATTATTCACGAATTTGAAAAGGCCATCCT |
| | | TACGAAGTACCCGGGCATCGATGGGAAGGACTTTGATAATATCATATCTCATTTTCAGCAGTCCAATAATCTTCC |
| | | GGAGCTGACCGCAATGCAAATAAAGAAGATCCGTAATGCTTTTAGCCACAATCAGTATCCTGATCCGATCTACT |
| | | TTCTGAATAACGAGGACTTGAAACTGATCGGTACTACGGGAGTAGAATTGTACACGGATTACTTCTTCAACTTCA |
| | | TCAAGACAATATTAGTGTCAGAGAATTAA |
| 363 | JW13188 | ATGGAAACGCCCGCGACCCCCAATTATAAACGGTTTGATTATGAATCAGATAAAGCATTTTATGCGGCTTTTTTA |
| | | AACACCGCCACACAGAATGTGTTTATCATCTTAAGAGACATTTCGGAAAGCTTAGGTTTGGGGTTTGACCTGTCA |
| | | AACGACGACAATATGCTGAACGTTAACCTTTGGAATATTTTGCAGTCTAACATAGAGCCAGAGTTATCACTGCA |
| | | CATAATTGAGAAAATTGAACGACAGTTTCCTTTTGCGAATTATCTGGCTAAAAATTATGCCTTTTACAAACGCAA |
| | | AGAACGCACTGCGGAACCTAATGATTACTGTAAAGTATTCGAGATGCTGATTGGACAATTGAACTCGTATCGAA |
| | | ATTATTATAGCCATGCAGTGCATGACTATGTACAAATGGATCCAACAATAATTAAGGGTATGCAAGTATTATTTG |
| | | ATGCTGCACGTCGAGAGGTTAAAGTTCGTTTTGCACTTAAGACAAAAGATGTTGATCATCTGGTGCGGTTAAGTT |
| | | CACGCAAAGTTTTTGGCAAAAACGAAGCCTATGAAAAGGAAACTTTTGAATATCGCTTTTTGGATGCGTCGGAA |
| | | TCTATTACTGAAAAGGGCTTTGCATTTTTCATTTGTATGTGGCTCCAGCGCAAGAATGCTCAAGAGATGCTGAAA |
| | | AAAATGACCGGATTTCAGAAATCCGATTCACCGTCCCAAAAGGCTACCTTAGAAACTTTTACTTTTTATTCGGCA |
| | | CGCGTACCGCAACCTAAACTTACCAGTGACAATACAAAGGAAGGGCTGTTGTTAGATATGATAAACGAACTGAA |
| | | GCGTTGCCCGAAAGACCTGTATCCGCTGCTGAGCAATACCGACAGAGAGAAATTCAAATCAATTGAAACGGAC |
| | | AAGGATACCAGCTTTGAAGATGAATATGAAGCTATGCCGATTTTAAAACGGTCTAGTAATCGGTTTAACTATTTC |
| | | GCGCTGAAGTACCTTGATAACTCTTTTGAGAAGATTAAGTTCCAAATCGATCTTGGTAATTATTGCTTTCACGTTT |
| | | ACGATCAACAAATTGACGGCGTTGTTAAAAAACGTCGCTGGATTAAAAAAATGTTAGGGTTTGGTGTGCTGGAA |
| | | GATTTTGCAGATCGTAATAGACCAGACGAATGGAAAGAAAAAATTCAAAAAATTGAAGGGCGTCATCATAAAA |
| | | GTGATGATGTCTATGTAGAGGAAACAACCCCGCACTATCACATTAATGAGCAGAACATCGGCCTGAAGATCTAT |
| | | GACAATTACCTGGAGTCTAAAAAGGAACGAAAAATATGGACGCCACTGCCTGAATTTGCTAAAGAAGATAAAC |
| | | AAAGACATAATGAAACGAAACCTCGCAATGAAAAACCGGATTGTTGGCTCTCACTGTATGAATTACCAGCCATG |
| | | GTTTTTTATCAGATTTTATTCCAGCGTGGTTACTCAGTTCTGTCCGCTGAAAAAATTATCGAAATACACAAGGAA |
| | | CGCATAGAGCGTTTTTTTGAAGATGTGGAAAACTCTAGACTTAACCCGGGAATTACTAATGATGCTTTACTTGCC |
| | | GAACTGGAGCGTCGTAATCTGGAAAAGTCCTTCATACCCAAAGCAATCATTAACTATCTTCTGTCGAAAACCAC |
| | | TGAAAGCTTTGAGTCTCTGGCTGAAAAGCGGCTGAACGAGTTACGTTTAGAGACTATCGATTACCTTAACATCAT |
| | | CAAACGTCAGCGCATTCATTTCATGGATAAACCGGGCAGTAAAAATTATATTGAGATGAAAAGTGGAGAGATG |
| | | GCAAATTTTCTCGCACATGATATGATACGTATGCAGAAACCAGTTGGTAAAGAGGATGGCAAAGCAAATTCTAC |
| | | AGAATTTCAGCTCTTACAGGCGAAACTGGCCTTCTTCACAGTGAATAAAAGTACTCTGTTCGAGACGTTCAAACT |
| | | TTGTAATCTGGTTGATGCAATTAATGCGCACCCGTTCCTGTTCAATATCAATATCTCTGAATGTAATGGAATTCTC |
| | | GACTTCTACATTAACTATTTGGAGCAAAAGGTCGCCTACATTAACCTTTGCAAACGGGAAAAGCGTTACGAAGA |
| | | TTATCATTTCCTGAAAATTGGTGAACGCCGGCGCAGCTCGGGGAAGGAATATATCGGCAAGCTTAGCAAAGAGC |
| | | TGAAAGATGATGCCGTGATGAACCTTCCTCGTGGGCTCTTTCTGAAACCCATTCTGGAAGCTCTCGATTCCAAAG |
| | | ATAAAACGAAACGGCTGGTGACCGAGTTCAAGAAACTGGACAGAATTAATGCCGCGTTTATTATCGAAAAATAC |
| | | TTCGCGTATGTTAAGGCAGATAAACCGCAGGAATTTTACTACTACAAGCGCAGCTATGAGTTGCTGAACAAAAT |
| | | CTTAGATAACCGCAAAGATTTTGAGAAACGCAAAACCATCGCCAAACAGTACTTTGACACAAACGAATTAACCC |
| | | AGATGACGGGCAAAATTGAAGGTAATCCTAAAGAGAATCCGATTACGCGTCTGTTACCAGTCCTCGTACGCAAC |
| | | CATATCTTCAATAACCGTATCAAGAGTGAGGATGAGAAATTGCGGGTCAAAAAGGTCTACTTTGAAAAATATAA |
| | | AGACTTTACGGAAAGTGAGAAACAGTTTAGACTCGCGAAAACTTGCGACATTGTGCTGTTCATGATCGCCGACG |
| | | ATATGTTCCGAAAGAACTTCGTGGTGGATAAGAGCATATTGAAGAATAAGAACAAAGTGTCCGTTCAGGATGCG |
| | | GTCGAAATCGGAAAAGGCTATAAACTTAGCGAAATAAAACCAAATTCTGACGAAGGTTTTTTGTCCCTTCAGAC |
| | | ACGTGTAGAGCTTCAGATACAGGACAAGACAATAATCAAAGAACAGATAAAAATTAAGAATTACGGTGACTTT |
| | | CGTGGCCTGTTAAAAGATCGGAAAATTGGATCTCTGCTGCCTTTCATAGCCGACAAAGAAATTCAGTTTGAAGC |
| | | GTTACAGAAAGAATTAGAATTCTACGATAAGGCCCGAATAGACGTCTTTGCTAAAATTTTTGAGTTCGAGAAGT |
| | | CAATCATCGCAAAAATGAATCTCCAAAAAAACCCCGAGGGTTTTATTAATCACAAAGACATTCTTAAGTCCTTG |
| | | ATCTCAGAGAATGACGAGCGGTTTATCTTGGTCATGCAGTTGAGAAACGCGTTCTCGCATTCCAATTATCCGGAC |
| | | TACAGTTTGTTCAAAGATAAAGTGGCCGATTATGGTTTTAATGATCTGAAGAACCATACAAGTAACGATGATAC |
| | | CATCCTGGTCAAAAGCTTTGCCGTACAGTTTAAAAATTTGATACTCTCATATTATGATGAATTGAGCCGTATATA |
| | | A |
| 364 | JW13 | ATGCGTATACCTCAGCTGATCGCTCAAAATAAACCAGTTTTTGGAACTTATGCAACCATGGCGCTTAAAAACGT |
| | 189 | GCAGATAGTACTTGATCACATCCAGAAAATTGAGTCTCTCCAGGATCAGATGCCAGAGCGCAGACCCGAGGATT |
| | | TTTGGGTGCATCCAGTGATGCAGTTTTTTTATTCCGAACGTTATGCAACGAATGTCGATCCGGAGAAAGCAGACG |
| | | CTATTAAAGAACGCCTGTTTCGTAATTTTCCCTTTCTTAAAATCATGGCTGAAAATCAGCGCGACTATCGTTGTA |
| | | ATAAAGATCACAAAGGAAAACGAGGGTACCGAAATAATAATATCGACTCGTCGAATCTGGAAATCAACGGTAG |
| | | TGACATCTACTATGTTCTGAACAATATGTTTCGCGTTCTCAAGACGTATCGAGACAACACAATGCACGCCATAAT |
| | | TAAAGATGATAAAATTGACCGAAACGACAGTCCTTTTTTAGAGAAGAACGAGTCCCCACTCAGTTTCACGATTA |
| | | ATAACTATTACACAGTTGCTTTACGAAATGTCAAAGAACGTTATAGCTATGAAACTAGCGACCTTGCATTCATTC |
| | | AGGACTACAGAACAGACAAACGAACTCTGAAAACCGATTTTAATTTCTTCCTGTCCATGCAGGCACTGAACGGG |
| | | GACCCGACAGGACGTATTCATCTTAGTGGCGTAGGGGTCGTTCAGTTGATATGTCTCTTTTTAGAAAAGAAATAT |
| | | ATCAACTTGTTCCTGTCAAAACTGCCAGCGCTGTGGGGCGGCTACGATGAAAGCTCAGAAGAAGCACGCGTGAT |
| | | TCGTCGGTCCTTAAGCATCAATTCCGTGGTTTTACCTAAAGAACGCATTCGTTCAGATAAACGTTCTATGTCTAC |
| | | TGCCCTCGATATGATCAATGAGCTGAAACGTTGCCCTCGCGAATTATTTGATACTCTGAGTCAGGAAGATCAGG |
| | | ATCGCTTTCGTGTTGTTTCCTCTGATTATAATGAAGTCTTACATGTGAGACATTCTGACCGGTTCGCCCAATTATC |
| | | CTTGCAATATATTGATTCCAATGAACTGTTCAAGGGTATTCGCTTTCATGTTAATATGGGTAAACTGCGTTATCTT |
| | | TTTAGTGCGAACAAAAGTTGCATTGATGGTGGTGTTCGGGTGCGTGTACTGGAACAACCATTAAACGGATTTGG |
| | | ACGTCTCGATGAAATGGAACAATATCGTCGCCAGAATGATACCACCTATGGCGATAGCAATATCGTGATTCGCG |
| | | ACTTTGAGAATGTGCAACGCGATGCTGCTTGCGCCGATAACTACCCGTATGTCGTAGACACCTATACTCGTTATT |
| | | TGCTTAATAACAACAAGATAGAGATGTGCTTCTCCAACGATAAAGTGGTCCCAGATATCGAACTGGATGGGGGT |
| | | AAGTGGTATGTTGGCAAAACCATTCCGCACTGTCGCTTATCTACCTTAGAACTGCCCGCGATGATGTTTCACATG |
| | | CATCTTTTGGGTGCCGAAAAAACGGAGCAAAGAATCAAAAGTGTATATTACAATTACAAGAAATTGTTCACTGG |
| | | GCTTAAGGAAGGCACGTTGACAAAAGAAAATATCGATTCCTTTAATATATGTAGATCAGACATGCCGCAAAAAG |
| | | TATTGGATGCAGTAAGCGGCGAGGATAAAACAAAACGTGTGTCTAGCTATGTTGAAGATACCTTAAAGGAACTT |
| | | CTGGAAGAGACCGCTCGCTTAATCAAGAGATTTGAAGAAACCAGAAAAGCTATAGGTTCAAGTGCTAATAAAC |
| | | AGGGAAAGCGTAATTTTAAAAGTATTAGCAGCGGCAAGCTGGCGGATTTTCTGGCCAAAGATATCGTAAAGTTT |
| | | CAACCCTCATTAGAAAAAGATGAATATTATGGCACAGATCGGATGACTGGTCTGAACTATCGTGTTATGCAGTC |
| | | GTCGATTGCCATGTACAACAGTATGGGTGATAAGAAAGCGATGCAGGACTTTTACAATATGTTCAAAGCCGCTA |
| | | AGCTGGTGGAAGATAACAAAAAATCGCATCCTTTCCTCGCAGTCGCACTCAAACGGATTCCGAAAAACGCGATT |
| | | GAGTTTTACGAACAATATCTGAAAGCCCGCAAACAGTATCTTGAAAAGAAGATCGTAGAGTTCAATAAAACCGA |
| | | ACACGTTCGGATGTTTGCAGCCAATCTTCCGTTTATTAATATCGAGCGGAACAAGTGGGCCTCGCGTGATGAAG |
| | | ATTTCTATCAGATTTTAGGAGATAGCTACCTTGAAGTTTCACCCATTGAACTCCCTCGTCAAATGTTTGATGACG |
| | | AGATAAAAGCCAAGCTGAAACAGAAACCGGAAATGCAGGATATAGACTTTGACAAGGCGAACGTGACATATCT |
| | | TATTGGCGAGTATTTGAAACGCGTGCATAATGACAATTTCCAGGAGTTTTATGATTGGGAGCGTAACTATCGCTA |
| | | TATGGACATGCTGGAGTGTAAAGTCAATAACAAAAACTCAATAGAGAAACAGTATTTGACGATAGCAGAACGC |
| | | GAAGATGCGTGGGCCAATCGTGAACAAGGCGTGAAAAGATACACGCAATGGGCCAATGATAAAAAACTCTCAG |
| | | ATGCGAAGTTTAAAAATATGCCTGAGACAGAGTATGCTAAAATTGTTGCAACCCGGCTGTCTAGCTCAAGAAAC |
| | | GATTACCAGAAATCGGAAAAACATATTCGACGGTACAAAGTACAAGACGCAATGATGTTCTTTATGGTCAAAGA |
| | | TTCATTAACTAAGAACATCAATTTCGTGGCGAAAGATTTCAAGCTGCAGGATATAATGCCGGATGCAGATAAAG |
| | | GTATTTTAAGCGGGATCATGCCTATGGACTTCGTTTTTGAGAAAAAAGGCAAGGTCTACACCATTAGCTCACAA |
| | | GGGATGAAACTGAAAAACTACGGGGACTTTTTTGCGTTAGTTAACGACAAACGGATGAGTTCTTTACTGGAACT |
| | | GCTGCCGACGCCGCAGATAGGTAAAGAAGATATTGAACAGGAATTCAAAGGATATGACGACAGCAGAGTAGAC |
| | | GTTGTGTCTGTGATTTTAGATTTCGAGAAGTTCGTCTACAATAAGTACCCGGATATTAAGAATCTGGCTTTGCAG |
| | | GGTAACCATTTCGATTTTCTGGATATGTTGAAGGAACTGATAAACAAGGGGGACCTTACAGAGAATGAGAAAGA |
| | | ATTGCTGCGCAAAATACGGAATGCGTTTTCGCACAATTCTTATCCGGAAGGTGGCATCGTCGAGATTCGTACGCT |
| | | GCCAGAAATTGCAAAACATCTGATCCGTGTATTTGGTAATTCTATTGAACGTATCCAGCATTAA |
| 365 | JW13190 | ATGGCTAATTTTCAAGAACCGGCACGTCATATATTTGGTTCCTATCTTAACATCGCCCGTGCAAACTTCTACAAA |
| | | ACGATATTCCACATCTTCTCAATATCAGGGATCGACTGCTATACGCAAAAGGGTGACTTGTATTTCAAAGAAGA |
| | | TAACCTGGACAAGGTGATAAAGGTATTCTACCTGATCTCGAAAGGCGAAAGCACCAAAGATGAAGGGACAAAA |
| | | GTATCACTCGAAGAAGAAATCAAAGCGTCGATCGATACCGTTTATGAAGTCATCAAAAAATGTTATGATAACCG |
| | | CTGGAATAACGAGGTTAAACAGTACACAAATTTGACGGAACGTGAAAAAAATGCACGCAAAGAGGCTTTCAAA |
| | | GCCCCTTTGACCGATGAAGGACCGAATGGGGAAAAAGCAAGAATTCGTAATACCTTTACAATTAAAGGAGAGC |
| | | AGGAAGAACGCTTCCGCAAAATGTTATTTCGACACATTCCTCTGCTGGCTCCGATTATGGCGAATGTTGTGGCAA |
| | | TGCAGTTCAAGAAGAATGTTAATGAACAGAAAGAGAGCAATAAGGCGATGCATGACGCGAGCCTGACGGACTG |
| | | CTTGAAAGAGTTATATTCGATTGCACAGTGTCTCACGGATTGCAGAAATTTCTACACCCACATGAATCCCTACAA |
| | | TAGTATTGAAAAACAGAAGGATCAGTTCAAACTCCAAAATATCATAGCCGGCAATTTGGATAAAGCGTTTGCTG |
| | | CAAGCCGGCGGATCGCCAAAAAACGTAACAGCTATTCTGAATCAGATCTGACTTTTTTAACGGGACATCAGGAT |
| | | GACTGTCGTATGCAAGAAGTGTTTGTCCTGGACGAAAATGGGAAAAAAAAGTGGAAAGTCGAAAAAAACGAGA |
| | | AAGGCGAAACCAAGCTTGACAAGGAGAAGAACCCTATCTACATCTACAAGAAAGTGAAAGCTAAAGACGATAA |
| | | AGGGAAAGAAAAACGGGATGAAAATGGTAAACCCATCTATGAGGTCCTTTATGAAGATGGAAAACCAGTACAC |
| | | GAGTATGAAACTAAGTTGATCGAGCGCGAAGATTTTTATTACAGAATACGCGGGATTCGGCAAATACTGAACCC |
| | | GAATCTGACTCCTAATGGCGAAGTCTGCGATGGATTGTCTGCTTTTGGTATGTTATACTTTTGTTCACTCTTTCTT |
| | | AGTAAGGAACTGACAGTACAACTGTGTACTGAGACGCAAATTTTCGTGAACAGTCCGTACAACAATGATAACAA |
| | | TAAGAAGAGCAATATCATCTTAAACATGATGTTCGTTTACGCGATTCACATTCCGCGTGGCAAACGACTGGATA |
| | | GCGAATGTAGTTCTCAAGCCTTAGCGCTGGACATGTTGAACGAACTGCGTCGGTGTCCGATTGAATTATACAAT |
| | | GTATTGCCAGCCACCGGGAAAAGAGAGTTTGAAGACGATATTAAACATGAAAACGACCGGACTCCAGAGCTCG |
| | | CAAAACGCATACGGTCCAAAGATCGCTTCCCCCATCTCGCTCTGAATTACATCGACAGTCAGGGCCTGTTTGAG |
| | | AACATTCGTTTTCAAGTTCGTTTGGGGAGCTACCGTTTTTGCTTTTATGATAAGATATGTATAGATGGTAAAAAA |
| | | CATCCCCGTCAGCTGCATAAGGAAATCAATGGCTATGGTCGTCTGCAGGATATGGAGAAAGCACGTAAAGAAC |
| | | AGTATGGCCATCTTTTCCAACTGTCCCGCGATGAATCTGTATGGCAGAAAGATGAAAATGCTTATGTTAATCTTC |
| | | GGCAGCTTGTCCCGGTGAAAGCTGGAGACCCCGCGCATATTACAGACACCTTAGCGCAGTATAATTTTTATCAG |
| | | AATCGTATTGGCCTGTTCTGGAACACAGATGACGAGACTTATCTTACTGACAAAATCAATGCCCAGGGCCAAAT |
| | | TGTCGGTAAAGGTTTGTATCTGCCGCCTTTATCTGCAGTGGATACTCCAACAACCACCAATCGTTATAAACGTAA |
| | | AGCACCGATTGATATGCCAGCTCCTCTGTGTAGTCTGTCCACTTACGATCTGCCTGCTCTGCTGTTTTATGAACAG |
| | | CTGCGCCGTAAACACCAGTTAGAACCAAATGACTTCCCGGCAGCGGAAGACATCATTATTCGCCAATATGAAAA |
| | | TATGGTGCGTTTTTTTACGGAAGTTAAAGATATGGGCACATTTGATAATGACGATGCCCTGCAAGAGAAATTAA |
| | | AAGAGTATGGCTTATCGAAAGCCTCTATTCCGCAGAAGATCTATCGCCACCTTGTTCAGAAAACCCCGGATAAT |
| | | GCGAAAGACTTTGAGAAATCCGCCAAAGCGGAGCTGCGCCGTCGTTTACATATTGCCGAGGCGAAACTGTCTTG |
| | | CTTCGATAAGGATAAAGAGAAAATGGAATCAGACAAAGAAGATGACCGGAAGTACAAGAAATATGGCAGTAA |
| | | GTACTTTATATCGATTCGGTATGCCCAACTGGCCCGGTATCTTGCTGAAAGCATAATGGATTGGACACCGAATGC |
| | | AAAACAAGGTAAAGATAAACTGACAGGATTAAACTATCGCGTTCTCGTTTCATCCTTAGCGGGATTTGGTACTC |
| | | GTTATTCTGATGAAGATATATTACAAATTCTGGCGGACGCGCACATCTATGTGGATGAGAAAAACGACCACCCC |
| | | TTTATTGGCTACGTGTTTGATGAATATCCAACAGATATTGAAATGCTGTACTCCGAGTACCTTAAGCGAGAAATT |
| | | AAAGAGATAAAGAAGTACATTAGCGTGGGTATTGATGCCAAAACGGGTGAGGAAACCATTACCTTGAAGAAAG |
| | | ATGCAAGTGGTATTCCATTTCTGCATCATGACCGAAAACGATGGCAACAGTCTTCAATTGCAGAACTCACAGAT |
| | | CGCTATCTTCATATTAAAGAGGAAGGAAAAGCTGAACCTGGGAGAGCTACGTTATTATTACCGGACGGACTCTT |
| | | TGCGCCGTATATCGTCAAAATGTTAGAAAAGTGTGACTCATCTGCACTTGAAAATTGCGATGACAAGAGTAACG |
| | | CCGCATTTCTTATTAGCCGCTATTTCGAGAACAAATGTGAGAAATCTCAACCGTTCTATGACACCACCGAACCAT |
| | | CTCATTTTAATGATGAATTGCGGAAAGTAACTCCGTATAAGTATAGCCGAGCGTATGAGTATTTTAAACTGCTGA |
| | | ACGGCGGTAAGGATATCTATCTTCCTATTACTGAAATTAAACGAAGACTGGCAGATAAAGAACGGAGAATAGA |
| | | CGCCAATATCAAAAATCTGACCGATAAGGGTAACTATCAAACGATCGAGGAAGCCAGAGAAGGTCGTCGGCGC |
| | | AAACTTGAACGCGCAGTTAGAGATATGCAGGACAATGAGAGAGCTATTCGCCGCTACAAGACTCAGGATCGAA |
| | | TTCTGTTCATGATGGCCAAAAGTATGTTAGGGGATATTGTGAATCAGAATGCAGATCTTTTTAAACTCGAAAATG |
| | | TTTGCGAGAACAATTTCTTGTCACAGCGCGTAAAGGCCCAGATTAGCGTGAAAAGTAACAACCAGCAGATCACA |
| | | ATCCAGAAAGATGACATGCCAATAAAGGATTACGGTAAACTGTTTCGCTTATTAAACGACGAACGTACCGTCCA |
| | | GCTGCTGAGCTATTCCATACTTACGAATCCACCTCTCATTGATTTTGATCAGATCAGTGAGGAGCTCAAACAATA |
| | | TAACACCATACGTTCTTCAGCCTTTAAAAGTTTTCAACAGATGGAGCAGGAATCCTTTATTCAGCATGAACGTGT |
| | | CTTAACGGATCCTGAGAACAACGATTTTTATCAGGGCGGTAGACGCTACAAGAATAATGAACAAACCAAAGAG |
| | | AATGATGCGAAACGCAATAACTTTTCGTCATTGCTGAAGATTCTGCAGTGCCTGACACCCGAGGTGATGAACAT |
| | | GTTTGAGGAAAATGATCGTCAGTTGATAACAGCTATTAGAAATGCCTTCGATCATAACAGCTATCCCAACTGGG |
| | | AAGTGTGCCGCCAGTTACTTGAGCAAGAACAGCAAGTTAATGCGAACATTCAGCTCGAACTGTCCCAGGTAGCT |
| | | GCATTCTTAATCTCGAAGATCCGTAACTATGTTAAACAGATAGAAGAAGTTTGCCATTCGTCACACCAGTAA |
| 366 | JW13191 | ATGCCGCTGCGCTTTAAAAATTCAGAAGAACGGAAGCCGGTCCTGGGAGCCTATGCCGCTATGGCCCGCAATAA |
| | | CGCTTACATTATCCTTAGCGACATCATGGAGCGCTTACATATTGAGCAACCGCCACTCCCAGACCCAGCTCACCC |
| | | TGGCCAATGTTTAGATAAAGAAGCACATCTGGACAAACTGAGCCTCTTTCCTCAGAATAATGATCTCATGCCGG |
| | | AAACAAAGGATAAAGCAATGAAGCTGCTGTGCTCCCATTTCCCTTTCCTTCAGACTGAGGGTGATCTGCGTATTA |
| | | TGAATGCTCCAGATGGGCGTGAACAGGAGAAAATCCCGATCACCTATGAGGAAAAATGTCGTACTTTACTGGAT |
| | | GCATTGGCTGTTTTAACTTATCTTCGGGATGCGAATCTTCATTATCTTTTTAATGATCGTCGCGCTGAACACCTTG |
| | | AATATCGCCTGCCTGAGAGCAAAGCTTGTCATCTGTTACGTCTGGTGTTAAAAGCGGCGCCGCGCAAAATAAGC |
| | | GAACGGTATAAAGGCACTGGGTTAATGCCTGATGGCGCCCTGGATTTTTTTATCAAAAAGCAGTATGTGCGGGA |
| | | AGGTCGTTCGTTCAAGTTCAATATGAATTGGTTGTTTTCTCCGCAGCGCGAGCCGGAAAAACGGGAAATGGATG |
| | | TTTATCGAAAAGTGGAATTGGCCGGCGGTGTGGAGAGAGATCTTGTTGCAAACCCTGACGGAACCCCAGCCAGA |
| | | TTCAATCGGTTAAGCCTGTTTGGGGAAGTTTTACTGATAAGTTTATTCACCGAAAAGCGGTACATACCAGACTTC |
| | | CTTAAAGCCTGCGGATTGTTGACCGATAATGGCTTCACGGAAACTACACAGGATGGTACATTGTCCCAGCAGCG |
| | | GCTGATTCGTGAGATAGTTGCAGCGTATGGCATTCGCCTTCCCAAACGTCGATTGGACATTACTATGGATGCGGA |
| | | CCAAGTAATGCTGGATATCTTCAATGAGCTCGCGAAGTGTCCTGATGAGTTATATCTGGTTCTGCCCGAGGCCAA |
| | | AAAAAAGGAACTCGAGGCAGTCAATATCGATGGGGAGACGGTTATCAAAAAACGCCACAGTGACCGTTTTGTA |
| | | CCACTGTTGCTGCGGTTTATCGACACGAACGAGTTGTTCCCCTCTATTAGATTTCAAGTGAACTCGGGGTATCTT |
| | | CGTTTTCTGCATCACGAAAAAGCAGCTTATATGGATGGCGTTGAAAGACCGCGTATCCTGCAGGACGCCGTGAA |
| | | TGGTTTTGGACGCATTCAGGAAGTTGAACGGACGCGTTCAGCATCAGATACGTATCTTGGATTTCCATTATACGC |
| | | ACCCTCTCCTGATGACGATGCCCCAATGTCTCTGCCCTGTATTACCGACTCAGCGAGCCGCTATATTGTTAACGG |
| | | TGAATGCATAGGTTTATCAATCTCTGGGGATCGCACACCAGTTATTATTCAAGACGGTGGTAAGTATCGCGTCCG |
| | | CTCCCCCTTACCGGATTGTTGGATTTCGCGATACGAATTGCCGGCTATGGGGTTTTATTTGTATCTGTGCAAAAA |
| | | ATACGATCTTAGTGTGAGCGCGGAGAAGATCATCATTGATAAAGTAAACGAATACAGATCTTTTTTTCAAGGTG |
| | | TCGCCAATGGGACAATCACTAGTCTGAAAGGTATAACGCTGGCGGAAAAGGACATTCCTGGTAAGCTGTTAGAC |
| | | TACCTTAATGGCAAGACCGACAGTTCAGATTTTAATCGCTATAAGAAGAACCTTATCCAGTCCTTACTCGAGAA |
| | | AACCGAAAATCGTTTGAAACGTTTTCTCGAAGATATGGCAATGGTGGGCAGTAAAGAAAATCGAATAGGCAAA |
| | | CGTAGCTTTGTGAGAGTTTTACCGGGTAAACTGGCATCCTACCTGGCAGATGATATTGTATTTTTCCAGTCATAT |
| | | CCGGCGGGCCATCCGGAAAAACGTCTCACAGGTCAGCAATTTTCGATTCTGCAGGGCTTGCTGGCCACCTTCCC |
| | | GCCTGATATTGAAGATTCTTGTCGCCGTGCGGGACTCCTGTCCGGTGAAACAGCCCATCCCTTCCTGAAAGCTGT |
| | | GTTCGATTCGATACCAGCCAAAGATAGAAACACGTTAGTATTTTATGAGATCTACCTTAAACGAAAGATTGCGT |
| | | TTCTGAAGGGCCCTATACCGGACAGCGCGCCCTTCCTGCACCCAGGAAAAGTCAAGTGGCAAAAAAAAGATGC |
| | | GGCTTATTATAAAGCCCTGGCACAGAGATATCTTGATGGTGGCAAAGACGGCTCTAATACTGGTATTTTATTACC |
| | | CCGTGGACTGTTTGATCAGGCGATTACCGATGTTTTAACCAAACGTTGCCCAGGTTGGTCAAGAGCGATCCAGA |
| | | ATACTGATCATGCTAACACCGCCTTCAAGATTATCACGTATCTGTTGGATGGGCAAGCAGATGACTACCAATGG |
| | | TTTTATTATGAAGAAGACCGGTTACAGGAGTATCGATTTTCCCAGTTAATAATTAGCCAGAACGCTGTAAGAAA |
| | | CGGATGGAAAGATCTTAAAGCCATTCTGCAAGAAGAGGGCGTTAAGCTGAAAGGTAATTACTACAAAGAGCTC |
| | | GAGGGTCGTATATCTGCTCTCAGACGTATTCCGCCGGAACGGCTGGCTCGCAACACAATGAAACGCGAGGAGCT |
| | | CCGTCAGGCACTGCGTTCAAGCTATGACCGTATGTGCGAAAACGAAAAGTCCATCCGACGTATTATTGTCCAAG |
| | | ACATCGTGCTCTTTCTGCTGGCCAGATGGCGGCTGGATCCGCGAGAGAAATTAAACATGAGTATGATAGGTCAA |
| | | GATGAATCGAAGCTGATGAATCAAGTGGTAACCGTTGACACTTTATACAAAGGGCAGTATCGTATTCGCCAGAA |
| | | TGATGTTCGCATTAAAGATCAGGGGGTTCTGTTCAAAACATTGAACGATAAGCGAATTGACAGTTTATTGGATC |
| | | AGTTGGCGGTAGGCGAGGGAGAAGTGATTGACCTTGAAGACCTTATAGCGGAACTGACCCGGTATGACGATCGT |
| | | CGCATTCCTGTCGTCGCAAAAATTATGGAGTACGAAAAACGCGTACTGAAAAACCACCCTGAATTACGTGATCG |
| | | TAGTGGTCGTATGGATTTTAAAAGTGTGCAGCGTGCCGACGAAGCATCATCGCCGCAGAAAAAAGTAGTCTTAC |
| | | GCACAGTTCGCAATGCTTTTAGCCATAACGCATACCCGGATCGTGTGGTGAAAGCGGAAGGCGAAGAAGGAAT |
| | | AGAAATCTACAACCCGATCATCCCTGGGATGGCAGACGGCATATCTGGAACAACGAATAAACTTGTCGATGAAT |
| | | AA |
| 367 | JW13192 | ATGGGCAAAATAGTCAAACCGGGCGAGAAGATCCGCGAAATGAAAGAAACCTACAAGTTTCGCGTCAACAAAC |
| | | ACCCGTTCTCCATTGAAGAAGTTGGCAAAAATATCTGTGGCGCCTACTTCAACATCGCACGTCAGAATTTTTATC |
| | | GTACACTCCTGCACATTTTCAGCTCGGTTGGGATAGATTGCTACACGAGTAAGAATTCCTTGTTTGTAAAGGAAG |
| | | ATACTATGGCAAGTGCCCTCGAGGCGCTGCAGGCTATAGAGAAAGATAAAAACACAAAAGAAAAAGTGACGCA |
| | | GGCTATTGAATCGCTGTATCAATTAGTCGAGAATTGTTATGACATGGAGTGGGAGCTGTTTTGTCAAAAAGAGT |
| | | ATGGTGGAAAGATTTCATCAATTAACGCTGAATTGAAAAAGGATTTGAGTGAGGATCTGAAAAATCAGCTGAAC |
| | | ACAGAGAAGGAGAAGATTGAAAAAGAACAACAGGACAAGCAGGAAGAGTTTTACAAAGACATTGAAAATGAG |
| | | AAAAACCGTCTGCGCCAAAGTTTTATTCTGAAATCGGAACAGGAAGAAAAACTGCGGAAGCTGTTGTTCAAACA |
| | | TCTCCCGTTCCTGTCTCCTATCATGGCAGATGTGGTCGCATATAATAATTCACATGCTGAAGGTGACACCATCAA |
| | | CAATGGAGTAATGAAAGACGTTACCCTGGCCGAATGTTTGCAACAGCTGGTGACCATAGCAAAGTGTTTAAACG |
| | | ATTGTCGGGATTTTTATTCCCATTACAATCCGTATGACAGTGTAGAAGTTCAGATAGAGCAATTCAAAAGACAG |
| | | AATGATCTGGCAATCAAACTGGACAAAGTTTTTGTTGCTTCCCGCCGGAAAGTTAAAGAACGAAATCAGTATAC |
| | | GGAGACGCAACTCGAGTTTTTAACCGGCAAACTGCACTATAGAAATCTCGAAAAGAAAGAACGCGATAATAAT |
| | | GGGGGCAAAAACAAGATACCTGAGGAGCGCCGCTATTATTATTACCGTATTCGCGGCGAACGCAAGTTTAAAGA |
| | | TGTCGAAAATTTTGAGGACTATAAGGATAAAAAGTCTCCAGCGATGTCGGACTTTGGGATAGTGTTTTTCTGCAG |
| | | TCTGTTCTTGAGTAAGGAATACACGACATTAATGTGTAACCAATTAGAACTTTTTGATCACTCACCCTTTACTGA |
| | | AGAAGCGAACAATATAGTGCATAACATGCTGTACGTATATCGGATCCACACCCCCAAAGGCAAAAGATTAGATT |
| | | CTGAAAATGATGAAAACGCATTAGGAATGGATATCCTGAACGAGCTCAGACGCTGTCCGATGCCTCTGTATGAG |
| | | GTCCTGCCGCCGCTCGGGCGCAAAGAATTTGAAGATGATGTTCGGCATCAAAATTCCCGGACCGAAGAAATAAG |
| | | CAAACGTTTGCGTTACGTAGATCGTTTTCCGTCTTTAGTGTTAAAATACATCGATGCTCAGAAACTGTTCTCTCA |
| | | AATTCGTTTTCAGGTACGATTGGGGTCTTACCGTTTCTCTTTTTATGACAAAATTTGCATCGATGGAGAACCTCAT |
| | | CTTCGCATTTTACACAAAGAGATTAATGGATTTGGGCGGTTGCAAGACATGGAAAAACTGAGAAAAAGTGAATA |
| | | TGGCGCCTATTTTCCAAAAACGCGTCAGGATAATGTATGGCAGCGTGATGAAAATGCGTATGTAGATCTTGTGC |
| | | AGCTCGTTCCAAACAAAGCTGAGGACGATCCGCAACTGACTAATAGCATCGCGCAGTACAACATTCATCAAAAT |
| | | CGAATCGGCCTGTTCTGGAACCAAGAGGGAAATAGCATTTTGGTGGATAAAAAAGGAGAAGACGAAAAACCGT |
| | | TATATGAAGGTTATTATCTGCCGCCCCTGTCTGTTGTGAAACAGACTACTTCAGAGCCTGAAAAGGATAAAAAC |
| | | AAAGCCCTTGTGGAAATGCCTGCGCCACTGTGCTCGTTATCTGTTTATGATCTTCCGGGTTTAATTCTGTATCAGC |
| | | ATCTTGTTGCTACACAACAGGGCTGCGAGTCTGTGGATGACGTTATTATCCACAAGTATCAAAAGATGGTGTCAT |
| | | TCTTCAATGACATCATTGAAGGTAAACTGCAGCCTCAGGATTCAGTTGCGGAGCTGGACAAAAAGCTTAAGGAC |
| | | TACTATGATCTCTCTAAAGCTAGTGTGCCTAAAAAAGTTTATCGTATATTATCCAGTCAAGAATTCAACAGAGAA |
| | | GAAGATCTTAAGGCCTTTGCTCTGAACGCATTAACACGTCGTTTAAGAAATGCCATTCGGCGTAAAACGAGATT |
| | | TTCAGAAGACCTTGCCAAAATTGGTCAGAAAGACAACAAGTACGGCAAAAAGAATTTTGTTGATGTCCGCCACG |
| | | GGGCCTTAGGCCAGTATCTGGCTGAATCGATACTCGACTGGCAGGTTACTTTTGATGGTGGTAAAGATAAACTT |
| | | ACTGGTTTGAACTATAGAGTCCTGGCATCGTGTCTGGCAAAGTTTGGTGATGGTAACACCAACCTGAACTATATT |
| | | AAAACGATTTTACGCAAGGCCAAAATTCTTGCTAATGAGGGAGAAAAAATAGCCAACGAACATCCCTTCATATC |
| | | AAAGGCCCTGGACAATTATCCCGATAACATAGAGGATTTCTATTTGGATTATCTTGATGCCGAAATTGAATACAT |
| | | CAAGAAATATGTGGACATTGAGGAATCCGGGCAGGAGATAACCAAGGTGACATTGAAAAGCGATGCGGACTTG |
| | | AGTAAACTGAACTTTGTACATAGCGAACGTAAACGCTGGGTTAAGAGTGCAGCAGAAGATCTGAAAGAATTAG |
| | | CCGCGCGTTATTTGTATGTGGAAGATCGTAATGTGAACGGTGAAGTTGTGACGCACCGGGCTACATTATTATTGC |
| | | CAGATGGCTTATTCGCAAGCCCGATTGTAGAGGTATTAAAAAAAAGATATGAAAAAGATGACCCGATTATGGCA |
| | | GCGCTTAACAACGAGAATCTTAATCATAACGTAGATTACCTGGTTAAACTTGTCTTTGAGAAAGAAAATGCCGG |
| | | AGACACTAGCCAGCCCTATTATAACAGCAATTTTGAAGTATTACCTGTCTACAACTCTAATACCGTTCCAGACTT |
| | | TCTTCGCGAGAATCCTGGTAATAACAAGTATGTCCGTGGTTACGAACTGTTCAAAGCAATTCATGGTAAACCCAT |
| | | CTTTTACAGCAGTGACGAAATCCAGGTGTTCCTTTCTAAAAAAATCACCGATCGTAACACCGAGTCACCTCTGAA |
| | | AATTGTTAATCGCCAGGGAGAAGAATGCTGCAAACGTGAAATCCATGCGAAAATAGACGCTAATGTGGAAGGG |
| | | GTGCGTACCAAAGGTGATTTTGATTCTCTGGAAGAAGCGCGAAATGCGCGCCGTAGAAAGCTTGAAATACTGTT |
| | | AAAGGAGTGCAAAGAGTCCGAGAAAACAATTCGCCGCTACAAAGCTCAGGATATGATTCTCTTCCTGATGGCAA |
| | | AAAATCTTCTCTCCAATGCGATTGGAGAGTCAGACAAGTTCAAACTTTCAAAAGTCTGCGACGATGACTTTCTCG |
| | | ATCAAAAGATCAATGCCCACATCACTTTGCATCTTGATGATTACACGGTTAAGATTCAGAAAGAAGAGATGCCG |
| | | ATCAAAAACTACGGCCAGCTTTTTCGACTGTTGAGCGATAATCGAGTCATGACAATTTTAAGCTATCCGTCCATT |
| | | AATGAAGCCACTTTAGAATACGACGATCTGATTGAAGAACTGTCCATCTACGATGAACGGCGTACCTCAGTTTTT |
| | | GACGTGGCGCAAAAATTAGAGCGTGCGTCGTTTGTACAACATAAAGAGGAGCTGACAAATCCAGAAAACGATC |
| | | TTTTTTATTGTCATAACAAACGATATCGCGTCAACAGCGGCCGAGAGAATCAGCCAAAACGGAATAATTTTGAA |
| | | GCAATGCTGAAATTACTGCTCAAGATGACACCGCAGCAGCAGTCAATGTTTAGTAAAGTCGAACAGGACTTTAT |
| | | CAAAAATATACGTAATAGCTTCGGTCATAACGGCTATCCAATCCGGAATACCATCGAACGTATTGCCAATGGTA |
| | | ATGATCCAAAGTTTGCGGATTTGAAAGAGATGCAGCTGAAAGGTATTGCAAGCCTCTTCGCAGGGAAACTGTTA |
| | | GATTGCCTGAATGATAATAATATTGAATAA |
| 368 | JW13193 | ATGAAATTGTCACTGAAACCGGATTCACAGCTCAAACCTATCTTTGGAGCTTACGCTGCGATGGCGCGGAACAA |
| | | TATTTTCCTCACTGTGCTGGATGTCATGAAGCAGTTACACATACCCCAGGAGCCATTGTTCGACCCGCGCGGCAA |
| | | TGAGCTGGACCTTGAAGCCAATATCTGGAAACTGAGCTTATTTGTTATCCATCTTAAACCCGAGCAGTCTTCGAA |
| | | AGCGGATAAACTGTTACGTACTCACTTCCCTTTTCTGGACCTTCTGAACGATATCAAAATCGGATCAATTAAAGG |
| | | TGAATCGTTTCATTACAATGTGAGTTTCAAGGACACGAAACAGTTGTTGCTTAACGGTGTGAATGTGTTATCCCA |
| | | TCTTAGAGACATTAACCTGCATTACAAGACAAATGACGGGAAAATAAACGACTACTATTATCGGAAATATGAAA |
| | | ACGTATGGTCAAACCATCTGATAGGTCTGCTCAAGGCTGCCCCACGCGTCATCAAAGAGCGTTATAAACAGACC |
| | | AGTCTCCTGTCTCCACAGTCAATGGATTTTTTTACCAAAGATTCCTACCTTAAAAACACCGACGGTTACATTCTC |
| | | AACCCAGACTGGTTGTTTTGCCCGACACGAAAACCGGGCGACAATGAGCTTTACATCTACAAAGATAAAAAACC |
| | | TTATCGGGTGGAAAAGGAACGTTTTGATCGTCTCACCCGTTTCGGGGAGATTTTAACCATTGCTCTTTTCATCGA |
| | | ACGGCGGTACATCTCGGATTTTTTAAGTGAATGCGGTCTGTTAGGTCAGTTTCCGAGTAGCACAAATGACGGTA |
| | | GTATATCCCAAAGACGTCTGCTGAAAGAACTGATTAGTGTATATTGTATCCACCTTCCCAACCGTCGTCTCGACA |
| | | TTTCGATCGGTGAAACTCAGATCAAACTTGATATGTTAAACGAATTGGCAAAGTGTCCTGATGAGCTTTATATGG |
| | | TTCTGCCAGACACCATTAAAACAAAGTTCGAGATAATCGGTATCGATGGGCGTCCGGTCTTTATGAAGCGCTATT |
| | | CAGATCGCTATCCGGCATTACTTCTGAATTATTTCGACACGACTGGCTCATTTGAAAAGCTGAGATTTCAAGTTA |
| | | ACGCGGGCTATTTGAAATATGTTCATCATGGGATAACAGATTACATGGATGGCGTCGGCCGTGTGCGTATTATTC |
| | | AAGATGGTCTGAACTGCTTTGGTCGTCGCCACGAAGTCGAATCCAAACTGAAAGAACAGTTTTCGATTCATGGA |
| | | TTTCCGGTCATACGCAATAATGAAGAGGAAGCAAATATTGATTACCCGTTTATTACCGAAAGCGGAACCCGATA |
| | | CGTGTTTAACGGTGATATGATCGGAATCAGTCTGTATGGAGATATCATGCCACGCTTCGAGCAGTATACAGAGA |
| | | ATAATACCGTACGTTATCGTGTTCGGAATACTACTCCTGATTGTTGGATATCTAAGTACGAGCTCCCAGCCATGG |
| | | CATTCTATTATTATCTGTCTAAAAAATACAATGAAGGCGTTTCGGTTGAACAGCTGCTGTCTCAGACGACAGAAC |
| | | AATATCGCAAGTTTTTTCAGGGAGTTAGTGATGGCACGATAAAAAGCTTATCAGACATCGATATTCCCATTAAA |
| | | GCAATTCCGCAAAAACTTCAGCTCTTTCTCAAGGGCGAATCCAGTCACGAAGATTATAATAAGTTCAAAGAGCG |
| | | CGTCATCTCCAAAATGATTGAAGAGACCGATATTCGTCTGAGTCGATTAAAAGACAACCTGGAAGTGGTGTTTC |
| | | AACGGGATAATAAAATTGGCAAAAAAACACGCGTACGTTTAAAGCCGGGCGTGTTATCGGATTATCTGGCCAAA |
| | | GACATCGTTCGCTTTCAATCATTTCCGGACAATCATCCAGAGAAGAAGATGACCGGACAACAGTTTTCTATATTG |
| | | CAGGGTTTGATAGCTACTTTTAGCGAAGAGCTTGAAGTAAGCTGTAAACGAGCCGGTCTGTTAGATGGCCCTAG |
| | | TAGCCACCCATTCCTCTCCCGTGTCTTCTCACGTCATAGAGGGCAAATGCGGAGCACTATTGATTTTTATCAGGC |
| | | GTATCTTGAGGAAAGACGTGCCTATCTGAGTGGTAGTATTCCGGATGATGCTCACTTCTTGCACCCGAATCGCGT |
| | | TAAGTGGAGCCATGATAAACAGGAATCATATTTCCGGAGCCTTTCTACACGTTATATTAAAGATGAAATAACCG |
| | | GTGAAAGAATTGGCATTTTTCTGCCCAGAGGCTTATTTCTGAAGCCCGCTCTGAAAATTATCGCAAACAACTGCC |
| | | CTAAGACGGCCCGCTTCTTAAAAGAGCAGGAGCGCATTAACATGAGCTTTTTGATCCTGACGTACTTGGAAGAA |
| | | GAGTTGCAAGACCAGAATCAGGGATATTATTACGATCATCTCGAAACAGGTAATGGTGAATATGACTTTTACAA |
| | | AATTATTGATCGCGAGAAGAAAGAGTTTGGCGACAAACGGTTAGGGTATATTATTCGAGCAGAACGAGATAGCT |
| | | GTCAAAATTCTCTGTACTTCTTCCAGCTTAAAAACGCGTTCGGCCACGTGCGCGAGTCTGCGCGGCCTATAGCCG |
| | | GGACAAAAGAGCTGGCCGAGAAATTAAGAGCCGCGTACAATAAAATGTGCAAAAACGAAAAGAAGATTCGCC |
| | | AATATATGATCCAGGATGCTACTCTGTTTCTGTTATCTCGAGATGTTGTTGGGCCAGCAGGGGGTCAAATGAAAT |
| | | TGGCAGATATCGATCCTACAGGGAACTCCGTTTTAGAACAAACCGTAGACGTTACGACAAATTATAAGGGTAAA |
| | | ACTATCAGACAGAGAAATGTCAAGGTAAAGAATTATGGTGAATTATACTCTGTGCTGAGCGACAAACGCATTAA |
| | | CACCTTACTGGAGTATCAGCCGAAAACCGAAAACAGCATACCGTTAGAAGCAATCAAAGAAGAGCTGGTGTCTT |
| | | ATGATCATGAACGGGTGCCCGTAATTACGAAAATTCAGGGGTATGAACGCGCCATTTGTGAAGAAAAACCGGA |
| | | ACTGTTCTCTGATGAAGATGCGCGTTATGATTTTAATGACATACAAAATGCGGATACTAAGACGCCTAATCTGA |
| | | ATAAAGTAGCTCTGAAAAAAATTAGAAATGCTTTTTCCCATAATCAGTATCCTGCACCGGTTGTGAAACAGGGC |
| | | AATAAAGAGATAGTTATTTTTACCCATTCGTTGCCCGATGTCGCGCGTGAGATATCATCAAAAGCAGGAGAATT |
| | | AACGAACTGGACGCCAGAAGAAGGATCCGAATAA |
| 369 | JW13194 | ATGGCACGCACAAACTTCGTGAAGACTATTAACTATATTCTGCCTATCGCTGGGGTGCGTGGCAATTATTCAGAA |
| | | TCCCAGATCAATAAAATGTTACACGCCCTCTTCCTTATACACGCAGGTAGATCTTCCGAATTGAGCGTAGAACAG |
| | | AGTAATTGGAAGAAACGCCTGACTTTGAACGTAGAACAGCAGGCTCGTCTCCAGCGTCTGTTGTTCAAACATTTT |
| | | CCGGTCTTACAACCAATGATGGCCGACGCAGCCGAGCATAAAGCGTACCTGGCTAAAAATAAAAAAAAGAGTG |
| | | ACGTGCAAACCGATGACGATGTATTTGCCCAATTCAAAGGTGTTAGCCTCGCGGAATGTCTTAATGTAGTCGCA |
| | | CTCATGGCAGATACACTGACTGAGTGCCGCAACTTCTACACGCACAAAGATCCATATAATACACCCGAACAATT |
| | | AGAAAAGCAGTATGAGCATCAAGAATTCATTGCTTTAAAACTCGATAAGGTAGTCGTGGCATCAAGACGAGTAT |
| | | TAAAGGATCGGGAAGGTTTAACCGTGGGAGAGGTTGAATTTCTGACCGGTATTGATCACATGAAGCAAGTCGAT |
| | | AAAAAAGATGAGAAAGGCAACGTCATCTACGTCGAGGATCGTGGTACACGAAAAAAAGCGAAAGCTTTTGTGG |
| | | AGTACGATGACTTCTACTTCAAAATTACGGGATCGCGTACTGCTGGTGGTAAAGCATGCTCCGCCTTAAGCGACT |
| | | TTGGCTTATTATATTTTTGCATCCTGTTCCTTAGTAAGCCCTATGCCAAGCTCTTTATCGATGAATCCCGTTTGTTT |
| | | GAGTTTTCACCTTTTGATGAGAATGAGAACATTATCATGCAGGAAATGTTAAGCATCTATAGAATCCGCACCCC |
| | | ACGGTTACATCGCATCGATAGTCGTGACAATAAAGCTGCGCTGGCCATGGACATATTTGGAGAAATGCGCAGAT |
| | | GTCCTATAGAACTGTATGATCTTCTGGATAAAGAACATGGTCAGCCGTTTTTTCACGATGAAGTTAAACATCCCA |
| | | ATTCGCATACGCCAGAAGTGTCGAAACGGCTGCGCTTAAACGATCGGTTTCCCCATCTGGCGCTGAGATATATC |
| | | GATGAAATGGAGCTGTTCAAGAGAGTTCGATTTCAGTTACAGATGGGCGCGTTCCGCTATAAGTTCTATGATAA |
| | | AGAGTGTATTGATGGGCGTGTTCGAGTGCGTCGTATACAGAAAGATATCAATGGTTATGGCCGTTTACAGGACG |
| | | TTGCCGATAAACGTATGGAAAGATGGGACGACCTGATTCAAAAACGTGAAGAACGGCCGGTCAAACTTGAGCA |
| | | TGAGGAATTATACATCGATCTGGATCAGTTCACTCAGGACTCTGCCGATAGCACTCCGTATGTTACAGATCGTCG |
| | | TCCAGCCTATAATATACACGCCAATAGAATTGGAATGTTTTGGGAAGAATCTCAGAACCCTCAGGGCTTCAAGT |
| | | TTTTTGACAAAAACGGGATGTACATACCCGTCTTATCCGTTACAGACGACCATAAAGCTCCTATTGCCATGCCGG |
| | | CACCTCGTTGCACGTTATCCGTACACGATCTGCCCGCCATGTTATTTTACGAATATCTTTTAAGTCAGTTAGACCA |
| | | AGAGGCGCGCAAACAATATCATTCAGCGGAACAGATTATTATTGGCATTGAAGCTGATTATCGCAAGTTTTTCG |
| | | GCGCAGTTGCAGATGGCAATATTAAACCGTTTGCCAGCTCAGAGAAATTTGCGGATTACCTGGAAGAAAATTAC |
| | | CCATCCATGTTGTTATCTGATATCCCTCAAAAAATTCGCCAGTATCTTTCGTCTGAACATCTGTCTTATGAAGGG |
| | | AAGCCAGTGACGGTGCGGGAACGCCTGGTTGAAGTTACTCTGAAACATCTTGCTGAGCGCATGGAGCGTCTGGA |
| | | ACGGCGTTTGAAAAACTACACGGAAGACCTTAAAAAGATTGGCAATAAAGACAATAAAATAGGAAAGAAGGAT |
| | | TTTGCGGACGTGCGTCATGGAGCACTGGCGCGCTATCTCGTGCAGGACATGATGGAGTGGCAGCCGTCTAAGGA |
| | | TGGGAAAGGCGCAGATAAACTGACCGGTCTGAACTATAACGTTCTGACCGCGTTTTTAGCAACCTATGGTACAC |
| | | CACAGGCTTCCATTGCCGACTCAGATATTGCCAAACTTAGTTTAAAGGAAGTTCTGCGTAACGCCCATCTGGTGG |
| | | GTGGGGACAACCCGCATCCCTTCCTGGAAGAGGTGCTGGCTTACGATAACAAAAACATTGAAGATTTTTATCTG |
| | | AACTACCTTACCGAAGAAGTGAACCACCTTAATTCACGTATTCAATCTTTGAGCTCGAATCCGACATATAAGGCT |
| | | TTGGAAAGTCTGCCTTTTGTACATCACAATCGTATTCGATATAATGAACGGACGGACGAGGAAATTCGCGCTTTA |
| | | GCGGGACGTTATGCGACTATTCAGCTGCCAGATGGCCTGTTCATACCGAATATTGTTGACCTTTTAGAAAATCAG |
| | | TATGCCACCAATACGGATATGCAAAAAGCGTTATCATTTGAAGGGGCAGATCATCTTAATCCGACCCGTAATGC |
| | | GACGTATCTGATAAGTCAGTTTTACAAAACTGTGCTGCACGACAATGCACAGCCATTTTATCAGACCGATAAGA |
| | | GTTATACTCGGCAGCAGGATGGCCAACCGCCTGTTGCATTTTCATTCCGCCGCGCCTATGATCTTTTTACGGAGC |
| | | TGAACAATGACAAAGAAGATTACTTTCCGTTTGAGTTGAAGCCGCTCTTCTTAACCAGCGAGGAAATCCAGCAA |
| | | CGCTTAACTGAAAAACGCCTGGGAAAAGATGGTAACCCGATTCCTCAACGGGAGAATGGAGAAATTAAGAAAG |
| | | ATGCTCAAGGGAATATTATTTGGGAACGTAAAATCACCCACGATGTTGACATATTTGTCAATACTCTCAGCAAC |
| | | AAGGATTTGAAATGCTCTAAAAATGAGGTTAAAGAGAAACGCGACGCGAAACGCGAAAAAATGAAGCGTATGG |
| | | TGCATGAAATCAAAAATAATGAAAGAACCATACGACGATATCGGACCCAGGACATGATCCTGTTTTTGTTAGCC |
| | | AAGAAAATGTTCGTTGACCTCTTGGGCGAGCAGTCACGGTCCGTCGATTGGAAGCAACTGCGGCTGAGCAAAGT |
| | | GTGTAATGACGCATTTCTGAGACAGACACTGACATTTCGTGTACCGGTAACGGTTGGAGATAACACAGTCTACG |
| | | TCGAGCAAGAGAACATGTCGCTTAAAAACTATGGTGAATTTTACCGCTTTCTGACAGACGATCGCTTGACCTCAC |
| | | TGTTTGAAAACATCGTAGATTCAATAGAACCGGATAAAAAAACAGGGGAACGTGTTATTAGCCATACCGATCTT |
| | | ATGTCTGAATTGGCGGCGTACGATCAAAGACGAAGTACTGTCTTCCGTCACATCCAGCAGATTGAGGATATTGT |
| | | GATCTCGGGGAATGCTATTCTGGCCAATCCTGATTGTGATAATTTTTGGGTAGATGGCAAAAGAGGTGAGCTCG |
| | | CAAAACGCAATAACTTTGCAAGTCTGCTGAAAATGATCGATCTTATAAACACGACAAGCTTGACGGATGACGAG |
| | | CGTACACTGCTCGTAGCAATTCGGAACGCGTTCGGTCATAATAGCTATCGAGTTGACTTGAAAAAAATCGGCGG |
| | | TATAAACAATCTTCCCGAAGTTGCAAATTCTATACTCAAACACTTGGAGACCCTTAGTGGTTTCTCTGCTGAATA |
| | | A |
| 370 | JW13195 | ATGTCGAAAAACGACAACATTAAGAGCAAAGCCAAAGCATTAGGCTTGAAAAGTACCTTTCAGGTTGGGGATG |
| | | AAGTAGTTATGACAAGCTTTGGTAAAGGAAATAAGGCCATTGTTGAAAAAATTGTCAGAGGCACAGATGTGCA |
| | | ATCCGTTCCTACTGAACCAAATTTTTCAGCAGAAATTGAGGGAAAAAAATTTGACCTTGTGGGCCGCGCACATA |
| | | TACAAACTAAAAGTGATAATCCGCAGTATTCTAAAAAACGCACCGGCGATGATATGATAGGTGCAAAAGCAGC |
| | | CTTGGAAAAGCGGTTCTTCGGCGGTACATTTGACGACAATATTCATATTCAGCTCGCGTACAATGTACTTGACAT |
| | | CGAAAAGATTCTGAGTGTGCACATTAATAATATCGTATATACCCTCAACAACATTCGGCGTAAGGATAATGCCG |
| | | AAGATGATGACTTTATCGGTTATATGTCCACGAGAAATGATTATGACACATTCATTGAACCCCGTAAACATAAT |
| | | ATATCAGAAGATGCGGCTAAAAGTATTGACAAGAGTAGAGCATCTTTTGAAGAATATCTGCAGCCGCCTGTTAA |
| | | AGATTGCCTTCATTATTTTGGAAATACTTTTTTTGCGCCCCGTGAAATAGAAAAGACCTATACCGATAATCGTGG |
| | | TTTTGAAAGAAAGAAAAAGGTCACCGTGAACTCACTGATTGATGAAAAAGAAATCTACTACATCTTCGCTCTGT |
| | | TAGGGGGTCTGCGGCAGTTTTGCACCCATGACAATAAAAGCGGCCGAAATTGGTTATATTCACTGGAAGAAAAT |
| | | GGCATCAATAGTGATGCGAAGGCGGTTTTAGATAAGTATTACAATTCTGCGGTAGCTCGCATAGATGAATCTTTT |
| | | GTAGATAATGCCTCTAAGACCAACTTCAAACTGATTTTCAATGCGATGGATGTGTCAGATGAGGCCCTGCAGGA |
| | | CAATATCGCGAAAGCGTTTTACAAATTCACAGTGTGTAAGTCGTTCAAAAATATGGGATTCTCGATTAAAAAAT |
| | | TACGCGAGCAATTGTTAGAGTTGCCAGAATATGAAGGACTCAAAGATAAGCACTATGACTCCGTGCGGTCGAAA |
| | | CTGTATCAGATCATGGACTTTGTTATCTACCTGTCTTTCAAAGACGAGAAGTTCAAAAAAGATAACGAGGAGAA |
| | | GATCAACAACATTGTCAACGAGCTCCGCGCTACTCTGAACGAAGAAGATAAAGGGCGGGTTTATGCATCCTATG |
| | | CAGAAAGAATGAAATCAGAGTTGAAACCCGCTATAGCCCGGCTCAAATCAGATATTGACAAGATTAAGGATTC |
| | | ACGCGTTAAAGAGTTTGAACTGGACGCAAGCGTTAAGTATCGTCTGAGCAAAGTCGTTGAATCTGTGAGACTGA |
| | | AAGATCGAGCAACTTATTTTACGAAGCTGATCTATCTGACTACTTTATTCCTGGACGGTAAAGAGATCAATGATC |
| | | TCCTCACCACGTTAATTCATCAGTTTGAAAATATCGCCAGCTTTATTGACGTGATGAACGATCGTGGTATAGACT |
| | | GCCGTTTCTCCGATGGGTACAAACTTTTCGAGTCGTCTAAACAAATTGCCTTTGAGTTACGAAATGTGAACAGTT |
| | | TCGCCCGCATGACACGCACTAGTAAAGATGACGAAAATGCTACCCACATGATGTATATAGATGCTGCTGAAATC |
| | | TTAGGCACTGACTATACCGAGGAACAAATTGAGGAACATCTTAATCTTGAAAAAAAACGTATGATACCAGGAAC |
| | | CAAGAAGGCCGATATGAACTTTCGTAACTTTATTATAAACAACGTGATAAAAAGCAGCCGGTTTAACTACCTCG |
| | | TGCGTTACAGCAATCCGAAAAAAATACGAGCGTTAGCGGATAATGAGGGCGTTATCAGATTTGTATTAGGTGAA |
| | | TTACCTGATGCTCAGATCGATCGTTATACTCTGCTCTGTGGCTTTAATCCAGACGCTGATCGCCAGGAAAAAACA |
| | | GATAAACTGGCCAAAGCAATCACAGGTCTTCGGTTTAATGATTTTGAAAATGTAAAACAGGGCGCCAACACGGA |
| | | AGGGGAGTCACAGGAGTCTATTGATAAAGCACAAAAACAAGGGTTAATTTCCCTGTACCTGACGGTTTTGTATT |
| | | TGCTTACGAAAAACCTGGTATACGTCAACAGCCGTTATTTTTTGGCGTTTCACTGTTTAGAACGCGATGCCCAGC |
| | | TGCTGGGTAGTGGGGCTGGTCATCACGAACCGTATGTCGCGTTGACCCAGCGCTTTATTAATGAAGACAAACTG |
| | | AACGAACATGCGTGCGAGTACCTGAAAACGAACATAGCAAATTCCGATGAGTATACAATTCGCATTTTTCGCAA |
| | | CAATGCGGCACATCTTTCTGCAGTTCGTAATGCAAACCTGTATATCGACAAACTTAAAGAGTTCAAATCGTACTA |
| | | CGAAATCTATCACTTTCTGTCACAAGAGAACATATACGGGAAGTATTGTGTCGATAAAAAGTATGTGACGGCTG |
| | | ACGAGAATGGTAGCAAAACGATTTCTGTCAAAATCTCCAAGGATTATTGTCCGCAGGTCTATATTGATAAAAGT |
| | | CTGGAATATTTCGATAAACTGAACAAGTACGGTACATACTGCAAAGATTTCACAAAAGCTTTGAACTCACCTTTC |
| | | GGATATAATCTTGCCCGTTATAAGAATCTTAGCATTGAAGGCTTGTTTGATCGTAATCGACCGGGAGACAAAGG |
| | | TGAAAATACGTTTGAGGATTAA |
| 371 | JW13 | ATGAACTTCGAAGAAAATGGGAATAAGTATCTTTTTGGAGCTTTCCTGAACACCGCGTACGATAATTATAACGA |
| | 196 | GGTGATCAGCCATATCTATGATGTTATTCGCCGAATTACTAATAACAATCCTACTGAACAGAAAAGTTCATCTCA |
| | | GGCTTTAGCATCCGAAGAACCGATCTCTTCCGAGGAGAAAGAAAAGATGTTCTTTGTGTGCACTATACTGATAG |
| | | CAGGCAACGCGACATCTCGTAAAATCGAATCAATATTAAAACGGGCGTTTCCGTTTTGGGATGATTTTATTGAA |
| | | ATCCATGCATCCTTATCAGCCCAGCTGGATAAAACGGAGTCCATCTTTCAGCTGCAGAAAAACTGCGTAGAAGA |
| | | GTTATTCACCGCACTGGAGAGTTGGAGAAATTACACCACGCATTATATTCACCCAGAGGCAATTGCTTCTCCCAC |
| | | CATCAATTTGCTGCTCAAAAGTTGCTTCAACCAGGGAATTAAAGAGATCAAAAAACGTTTCAACACAAAAGATG |
| | | AGCAGCTGACACATCTGTACGAGCCCGAGTTGTTTCATAATAAAGAGACCAACAAAGATGAAATCAAGTGCTAT |
| | | CACCCGGCTAATTATTATCGTTTTGCTAATGAAAACGGTCTGCTGACCGAAAAGGGTCTGGCGTTTTTTACATGT |
| | | CTCTTCCTGTACAAACGCGAGAGCTATCTTCTGCTGAACCAGCTGAAAGGCTTCAAACGTGCGGATGAAGAACG |
| | | CTATCGGATGACGCTGGAAGCGTTTACTGTTATGCGCTTTCGTGCACCGAAACAGCTTTTGAAACTTCAACAGCA |
| | | GCAGTTGGATAAAGTCTCATTGGGGATATCGATGATTAATGAGCTGGCCAAGTGCCCGAAAGAACTGTATGACT |
| | | GTTTGGAAAATGAAAAAAGACGACAGTTCGAAGTTTACCCGGATACTGAAACTACGTTATATGAACAAGAAGC |
| | | AATTGGAGAACTCATTCGTTATAAAGATCGGTATGAAAATCTTATGCTCAAAGCTCTGGAGCATAGTTCGGAAT |
| | | GTAAGTACATGGGCTTTTATTTGTCCCTCGGTAAGTGTTATATGAAATGCTATGAGAAACATTATATTGACGGTA |
| | | CTACCGACAATCGTTATATTGTCCATAGCCTTTATGGTTTTGGGCGTTTACAAAATGCCTCGACCGATCATAAAA |
| | | ATCATCTGAGTAGCTTTGGTGAAGAATTACAGACACACAGCCAATTACCCGTCTGTCACTTTGCCACTGAGGATG |
| | | ATGCGGCTGATAGTCCTACAAGTGTTCCTTATATTTCAGAGTCATATCCGTCTTATGTTCTCAACAATAACAATA |
| | | TCGGGATTAAGTGGCTGAACTCTTCCGAAAAAACGGAATACCCGAATTTTAATCTTAATGAAGGAAAAGTTTCC |
| | | TGTCCTGCCCCTGATTATTGGCTCTCTAAGTATGAACTGCCGGCTATGGCCTTTTACGCCTATCTTAAAGAGAAA |
| | | CACAAGAATAAACTGCAGGAGTATCCATCAATTAAGGAATTAATTGACCAGAGTCATCCGGAAGCTACCAAAA |
| | | CTTCGAGCTCGACAATCAAAGATAGATTAGAAGAACGACTGAAACGGTATAAAGTGGACGCCACGAAAAAACT |
| | | GAAACGGTTAGCAGAAAAACCGGATGGTGTTCAAAGCGGCCGACTGGCGGATGAACTGGCCCGGGATATATTA |
| | | TGGCTGCAACCAAGCTCTCCTAAAGAAGGCGGTAAAGATAAACTTACCGGCGCCAATTTTCAAGCATTACAGTA |
| | | TGCACTGGCCCGTTATGATTTCATGCGCACCGAATTACCAAGAATATTAACCGCGGCGCGTCTGACGGGATCAA |
| | | ATAATGCGCATCCTTTTTTAGATAAGATTAATCCACAGGACTATAGCCGCCTCACAGCATATTACGCAGGCTATT |
| | | TGACGCAGAAAATTGTCTACATAGATAAGCAATTAGAAAAACTGGCTAGAGGGAAGGTTAACATCCAATTTCAC |
| | | CCGACGCGCAAACTTATTCGCCAGTCTCAAACAGATTCAGTGGAACATGAAAAGGAAACCGTCTTTTTACCACG |
| | | TAATATTTTTACCGAGAAAATTGAACAGGCCCTGTGCCAAATACAGGGCAATTTTAAACTTAATATTGAAAAAG |
| | | CCCGTATAAGCGGCAAGAAGCTGAACGCTTCTAAACTCATATTGATCTACCTGGAATTATATCTTAAGGATGAA |
| | | GTGCCTGAATTTTACAATGCTAAAAGAAAGTACCCCTGTCTGTGTTATCCACTGACCCCGAGCGAAAAATATACT |
| | | CTGAAGGAGTATCAGGACACAGAGGAACGGTCGAAGCGCATTGACCGTCTCAAATGTTTTTACAATTTTAAAGA |
| | | GGTAGCAGAAAAAGTCTATCTTCACAAGGAACTTAGCGAGGACGAAAACAAACGCTACAGAAAGTATATAGAG |
| | | GTTCAGAAAGTGATCGATAATGAAAAAATCATTAGACTGCGAAAAGTGCAGGATATTACGCTTTATCTTTGGAC |
| | | ACGTCTGTTCATTGAGGAAAATCCTGAGCAAATCTTTTTCAGTAAGAACGAAAAACCAGAGAACAGTCTGTTCA |
| | | AATTGCGGGAAATGAATAATGCCATACTTGACAATACCACGTCACGGAAAATTAATATTAACACTCCTAACGGT |
| | | AATGTAAGCATTGAAGACGAGTTTAAAATTAAAGATTATGGTCGCGTAACGGCACTGACAACCGAGAAGTTGCC |
| | | CCAATCTTTAATAAAGTTCATAGCGATTCTCAACAAACCCTTAAACAAAAAAGATCGCCCGTTATTGTCTTATGA |
| | | TTATATCTACCACGAGACAAATGCATTCTACCGCTACCGTCCGGAAATTATCAAAATGGCGCAGGACATTGAAA |
| | | ACAAAATCATCAACGACCTTAAGATTAATGTAAAACCACAAGCCGAATCCTACTACAACTTTCGTCGCACCATC |
| | | CAGAAGCTCGAGAACAAAAGTCGCGACATCAACATCGAATTGTTAATTAAAATCCGTAATGCATTCTGCCATAA |
| | | CGAGTATAAGGAATTAGACAACGACCAATACAAGTACCTGAATTCATTGTTTAAACTGTCGTCCGACATAAAAC |
| | | AGACTGATACACTGGCGTATTTGTTATACATTTTGTTCGAGACTGAGTACCAGAAAGTGATGATACTGAAATAA |
| 372 | JW13197 | ATGGCAAATACGCAGTTCAAACTGAAGTCTGTTGATTATTCAAAACCGTACGATGAACAGCCCGAATGCAAACG |
| | | GCATGTAGTGGGAGCCTATGCCAATCTGGCCCGACATAATATGACCCTGACTGTGAATACGATTATGCAAGCCA |
| | | TTGGGATGGAAATATTCGATGAAAACAATATTGGAGACGCGTTTAATAGTAGTCACCGTAAAAAGATCGACGCT |
| | | CTGGATAACATTCAAAAAGTGAATCTGCAGAAGCGGTTGTACCGTCATTTCCCCTTTTTTAAACGCATGAAACTC |
| | | GAAGATGAAGACAAAAAGTCGGTGCAGTTAACTACACTGTTAGAGGTTATGAGCGACTTCACATCATGCATGGC |
| | | GATTATACGTAATTTTTACACACATTATCACCCATATAATTCACCGGATGAGAAAAAAAGACAGCTGGAATTAA |
| | | AGAAACGAATGGGCAAACGCTTACAGTTTCTTTATGAAAATACATCCCAAATGTTCAAAACTACTGAAGCACTT |
| | | GACCATGAAAGTAATGAAGTGTTGTCCGCCCTGCGCATTCCCGAAGATTACATTGAGGAAATTTTACCGGGCGA |
| | | CAAAGAATACGACAATTTGTTTAACCTTATCAATGATCCTCGGACGAGTAAGTTCAAAAAGCAAGGCTTGAAAC |
| | | TCAACCAGCAGACCAAAGTGATTACTCGTAAAAAAGTTCGATTTGTACGTAATCCTGACTATCAGGCCTACATG |
| | | ATTGATGAGACAAATGGCATGAGCGACGTAGCAATTATATTCTTTCTGTGTCTTTTTCTGGACAAAAAGGTCTCC |
| | | TTCGATATGATGGACGAAGTGGGCTTCACGAAACAGATAAAGTTTAACGGTCCGCAGGCTGATCGTCAGCTGTT |
| | | GTATCTGAAAGAGATAATGTGTATGAACAGAATCCGTATGACCAAAGCAAAACTTGATAGCGAAATGACAGAT |
| | | ACGGCCCTGGCACTCGATATGCTCGGGGAAGTTCGAAAGTGCCCAAAAGCGTTGTATGAGGTATTAGATAAAGA |
| | | TGCACGGAATGAATTCAAAGATGACGCGACGGTTCAATGGGAAACGGAACACGGTAAAGAAGCGGTTTCGACT |
| | | GAAGCGCTCGCTTCTGAATCTGATGAAGACGAGTTAACCGACAAAAATACACCGCGCTCAACATTTGTTCGTTG |
| | | GGATGATCGTTTTCCTCAAATGGCCTTACGTTATATTGATTATCAGGGTCTTTTCAATGACATACGCTTTCAGTTG |
| | | AACCTGGGTAAGTATCGTTTCGCCTTTTACCAGCATGATAACAAACAGTCTGTTGATAACGAGGAAAGATTAAG |
| | | AATATTACAGAAAGAGTTACATGGGTTTGGACGCATCCAGGAAGTAGAAGAAAAAAAGAATGAGAAGTGGAAA |
| | | GAACTGTATGACGAAAAGACCGTGAAAGATGGCTTAACACAGAAAAAACCGGATGAGGCCGGTCAGGCCCCGT |
| | | ATATCACTGAGCAGTATGCACAATATGCTATTGATGAGAAATCTCACTCGATTGGTCTGCGTTGGGAAGGGTGG |
| | | GATAAAAAAGATAAGAGTAAAGGCCACTATGGTGATCTGGACAAACTGAAGATGTTTATTCCATATATCCCGAG |
| | | CAAACCTAAAGACGAAGAGCAGCAAACTAATCAGGCGGAAGATCTGATGGCTCCACAAGCCATGCTGAACCTG |
| | | TATGAACTTCCCGCTTTACTGTTTTATCAGTACCTGCTTGATAAGTACAAGTCAGATACCAGCGATGCGGAAGAC |
| | | ATAATTAAAACAAGCTTCACTAATCTGGTTAATTTTTTCAACGACGTCGAATCTGATGTCCTGGTTCCTATCGAT |
| | | GATCCTGATGAAAACGAACGCATTGATAAACTGAGTTCCTTACTCGAGGGCTATAATCTTCAGATGAGCGATAT |
| | | ACCGGAGAAGTTGCGTAACTACCTTTGCAACAAGGATATTGACTATGATCTCAAAATGGAGAAAAGCGCTATTG |
| | | GTCGGTTAGAGGAGAAGAAAAAGCAAGTTGACAAAGCGCTTTCTAGTTATAAGGACAAGAAAAAGATAATTGG |
| | | TTCAAAAGATAACAAGTTTGACAAAATGCGTGCGTCTATTAAAACGGGACAGCTGGCAACCTGGTTAATACGTG |
| | | ATATTATGGATTGGGTTCCAAATAACTCCTCAGCACGCAATCGACTGACTGGACAAAACTATACCGCTTTGCAA |
| | | GCCACAATGGCTCTCTTTGGCCAGCAAGCGAATAGCAACAGAAGTTATCTTGAATCCTGTTTTCGTTCCGCTCAT |
| | | CTTCTGCCATTAGACGGTAAACCTTATGATAAAAACACGCATCATCCGTTTCTCGAAGATGTGATTCAGGCGATG |
| | | GAAGACGAAACCTTAGAGTCGTTCTACGAGAAATACCTTGATTTTGAGACCAAACACATCGATAAAGTGATTAA |
| | | AACTCTCGAGAACGGGACAGATTTTGATGATTATTATAAAGTGCCTTTTTTGCATTGTGATCGTCAACGCTGGGC |
| | | AGGTCCCGACAAAAGTTTGGCCGCGCAGTATCTTAAACGACCGATTCAGCTCCCAAATGGGATTTTTACCAAGC |
| | | CCGTTTTTAATCTGCTGTCTACGATCGATAACAAAGCCTTAAGAAAAGCACTCGAGGAATCCCAGCAGGCCAAT |
| | | GGAGAAAATCCGCCGCTGGCAAATAACGTCGCGTTTCTGATGAAACTGTACTTTGAGTTCGTTGAGCATGATCA |
| | | TTCCCAACCGTTTTATTATACCACGACGTCAGACGGCAACAAAAGCCACTATCTTCATATCTACCGCATCTTCAA |
| | | AAAGTTGTACGGCCAACCAATACCACACTCAAACAAAACCACTGTACCGGCGTATACAATTGACGAAATCCGCC |
| | | ATCTGCGTAAACAGGCGATAGCAGACATCGAAGAGTATGTAGACAATGAAATCGAGAAATGGAAACGTACCAA |
| | | ACAAGACAGATTCGAATCCAAGCTGCGGAAAAAGCTGAAGAAAGAAAATGATAAGCGCTACAAAAAGCATGA |
| | | AAAGCAGCTGAACATCAACGAGGAGATCCAGAAAGTTGTGAAAAAAGAAATAGATACCATGCGCACAGATATG |
| | | ACTAGCAAAATGAAACGTCAACTGCGGTCTGTATATGATAACGAACGGACAATTAGACGGTTTAAAGCTCAGGA |
| | | CATGATGCTTTTAGTAATGGCACGGAATATCTTAAAAGCCAAGAGTAACGATAAAGATTTTACGGACGGTTTTT |
| | | GTTTGGAGAACGTTTTCAGCGATTCGTTACTTGATAAACCCATTGACTTCGAATGGAAAGTCAATATCAAAGGTG |
| | | CTTCTAAGACAATCAAGCAGGAAGGCATGAAAATGAAAAACTATGGTCAGTTTTACAAATTTGCTTCAGATCAC |
| | | CAACGCTTGGAATCATTACTGTCACGCTTGCCAGACTCGCTGTTTCAGCGAGCAGAAATAGAGAATGAACTGGC |
| | | TTACTACGACTCGAATAGAAGTAAAGTCTTTCGGCAAGTCTATATCATTGAATCGGAAGCATATCGTCTGAAAC |
| | | CTGGCTTACAGGATGATGCCAATGCAAGCAAAGATTGGTTTTACTTTACGGACAAAAAAACCCATAAGCAACGT |
| | | AGTATCCGCAATAGCTTCATACAACTCCTGGAAATTCTGGCGGCAGGGGAAGATGGAATTTTGGATAACGATGA |
| | | GAAAATTGCTCTGCAGAAAACCCGCAATGCCTTTGGACACAATACTTATGATGTGGATCTGAATGTCGTCTTCAA |
| | | GGGTAAAGAAAATAAAAAGAAGATCCCTGAGGTGGCAAATGGGATCAAAGAGAAAATTGAGACCCAGACCGA |
| | | AGAATTAAAACGCCGTTCTAAATAA |

TABLE D
Target Sequences

| SEQ | | |
|---|---|---|
| ID | | |
| NO: | Name | Sequence |
| 373 | FluA_ | CUUGAGGCUCUCAUGGAAUGGCUAAAGACAAGACCAAUC |
| | Target | UUGUCACCUCUGACUAAGGGAAUUUUAGGAUUUGUGUUC |
| | | ACGCUCACCGUGCCCAGUGAGCGAGGACUGCAGCGUAGA |
| | | CGC |

TABLE E
Direct Repeat Sequences
Table E - Target Sequences

| SEQ ID NO: | Name | Sequence |
|---|---|---|
| 374 | JW13101_Direct_Repeats | GACUGAACCUUACCAUGUAGGGAAUUGAAC |
| 375 | JW13102_Direct_Repeats | GUUGUAGAAGCUCUUAUUUUGAAGGGUAUAACAACU |
| 376 | JW13103_Direct_Repeats | AUGCCUUAAUUUUGGAAGGUGAGAACAAC |
| 377 | JW13104_Direct_Repeats | GUUGUAACUGCCCUUAUUUUGAAGGGUAUAAACAAC |
| 378 | JW13105_Direct_Repeats | GCUGUAGAAGCCUCCGAUUUGUGAGGUGAUGACAGC |
| 379 | JW13106_Direct_Repeats | UAGUUUUAUUACCCCUACAAACAGGGUUAUAUUCUAAC |
| 380 | JW13107_Direct_Repeats | GUUGUAACUGCCCUUAUUUUGAUGGGUAAAAACAAC |
| 381 | JW13108_Direct_Repeats | GUUGUGCUUACCCUACAAAAUUA |
| 382 | JW13110_Direct_Repeats | GUUGUUGAAUGCUGCAUUUUGGGUAGCAAUUACAAC |
| 383 | JW13112_Direct_Repeats | AGUCCCCGCUCGUUUCGGGGUGGUCU |
| 384 | JW13113_Direct_Repeats | GUUGUUUUUAUUCUUCAAAAUAAGAGCAGUUACAA |
| 385 | JW13114_Direct_Repeats | GUUGUCUUUACCCUCCAAACCAAGAGCAUUCACAAC |
| 386 | JW13115_Direct_Repeats | GUUGUGGGUAUUCUGCAAAUUUAGGGCAAUAACAAA |
| 387 | JW13116_Direct_Repeats | CGAAUUCCGAAUUAACCUCGUGUAGUGACGUCGUUC |
| 388 | JW13117_Direct_Repeats | GUUGUUUUCACCUUGCAAAUAUAAGGCAUUUACAAC |
| 389 | JW13118_Direct_Repeats | GUUGUAACUGCUCUUGUUUUGAAGGGUAAACACAAC |
| 390 | JW13119_Direct_Repeats | UGCUCUCAGUUUUGGAGGGUAAACACAAC |
| 391 | JW13120_Direct_Repeats | AUGCCUUAAUUUUGGAAGGUGAAAACAAC |
| 392 | JW13121_Direct_Repeats | GCUGUACCUACCCUGCAAAUCAGGAGCCUCUACAGC |
| 393 | JW13122_Direct_Repeats | AAGUUGUCAUACCCCUCCCAACGAGGGGCUUCUACAAC |
| 394 | JW13123_Direct_Repeats | GUUGUAGCAACCUAUAUUUUGUUAGGAAAAGACAAC |
| 395 | JW13124_Direct_Repeats | GGACUACACCCUUACCGUUGUGUAGGGGUCUAAAAC |
| 396 | JW13125_Direct_Repeats | GUUCAGCAUGACCGCUUCUUUUACAGCGGACUGAACC |
| 397 | JW13126_Direct_Repeats | GUUGUAACUGCUCUUGUUUUGAAGGGUAAACACAAC |
| 398 | JW13127_Direct_Repeats | UUCAAAAUAAGGGCAGUUACAAC |
| 399 | JW13128_Direct_Repeats | GUCUAAGACUAGGUAAGAAUUAGCGGUGUCCAUUGC |
| 400 | JW13129_Direct_Repeats | GCUGGAGCAGCCCUCGAUUUGCAGGGUAAUCACAGC |
| 401 | JW13130_Direct_Repeats | GUUGUAACUGCCCUUAUUUUGAAGGGUAUAAACAAC |
| 402 | JW13131_Direct_Repeats | GUUGUGUGUAACCUAAAAAAUGAGGGCAAUAACAAC |
| 403 | JW13132_Direct_Repeats | GCUGUAGAAGCCUCCGAUUUGUGAGGUGAUGACAGC |
| 404 | JW13133_Direct_Repeats | GUUGUAACUGCCCUUAUUUUGAUGGGUAAAAACAAC |
| 405 | JW13134_Direct_Repeats | GCUGGAGCAGCCCUCGAUUUGCAGGGUAAUCACAGC |
| 406 | JW13135_Direct_Repeats | GUUGUUCUCACCUUGCAAAUAAUAAGGCAUAUACA |
| 407 | JW13136_Direct_Repeats | GGUGUACCUGCCAUGCAAAUAGAUGGCUUUCACACC |
| 408 | JW13137_Direct_Repeats | GCUGUAAUCACCCCGCAAAACCAGGGUUGCUUCAGC |
| 409 | JW13138_Direct_Repeats | GUUGUCUUUACUCCACAAACUAAGAGCAUUCACAAC |
| 410 | JW13139_Direct_Repeats | GGUGUUUUAAUCUUUCCAACUAAAGACUUUUACACC |
| 411 | JW13140_Direct_Repeats | CCUUCUUUUUGAAAGGCACAAAUACC |
| 412 | JW13141_Direct_Repeats | GUUGUUUUCACCUUGCAAAUAUAAGGCAUUUACAAC |
| 413 | JW13142_Direct_Repeats | GUUGUUUUUACCCUUCAGAAUAAGAGCAGU |
| 414 | JW13143_Direct_Repeats | ACGUUGUCAUACCCCUCCUAACGAGGGGCUUCUACAAC |
| 415 | JW13144_Direct_Repeats | GUUGUGAAUGCCAUAUUUUUGAAAGGUAGAAACAAC |
| 416 | JW13145_Direct_Repeats | GUUGUAGCAACCUAUAUUUUGUUAGGAAAAGACAAC |
| 417 | JW13146_Direct_Repeats | GUUGUUGAAUGCUGCAUUUUGGGUAGCAAUUACAAC |
| 418 | JW13147_Direct_Repeats | GCUGUACCUACCCUGCAAAUCAGGAGCCUCUACAGC |
| 419 | JW13148_Direct_Repeats | UAGUUUUAUUACCCCUACAAACAGGGUUAUAUUCUAAC |
| 420 | JW13149_Direct_Repeats | UGCUCUCAGUUUUGGAGGGUAAACACAAC |
| 421 | JW13150_Direct_Repeats | GUCAUAGACCCCUUCAAUUUAUGAGGGGUUUUGUUC |
| 422 | JW13153_Direct_Repeats | GUUGUGGAAGCCCUCAUUUUGAGAGGGUAUUACAAC |
| 423 | JW13154_Direct_Repeats | GUUGUGCUUACCCUUAAAACUAAGGGCAGCUACAAC |
| 424 | JW13155_Direct_Repeats | GUUGUAAGUGACCUCAUUUUGGAAGGUAAAAACAAC |
| 425 | JW13156_Direct_Repeats | GUUGUGUAUAUCUCCCAAAUCAGAGACAGCUACAAC |
| 426 | JW13157_Direct_Repeats | GUUGUUUAUACCUUACAAAAUAAAGGCUCUCACAAC |
| 427 | JW13158_Direct_Repeats | CCUUUGGAGUACUGUUAUUUAACAGGGUUCUAAAAC |
| 428 | JW13159_Direct_Repeats | GUUGUCGAAAGCCUUAUUUUGAAGGGCUAUUACAAC |
| 429 | JW13160_Direct_Repeats | AUGACUAUACCAGCAAUGGCUGGAUUAAAAC |
| 430 | JW13161_Direct_Repeats | GCUGUAGAGGCCCCCGUUUUAGGGGGUAGGUACAGC |
| 431 | JW13162_Direct_Repeats | GCUGUUACUGCCCCACAAAUCGAGGCCUAUCACAGC |
| 432 | JW13163_Direct_Repeats | GCUGUAGAGGCCCCCGAUUU |
| 433 | JW13164_Direct_Repeats | GUUGUGCCUACCUCUCAAAUAUAGGGCUUUCACAA |
| 434 | JW13165_Direct_Repeats | GUUGUAAAUACCCUACAAAU |
| 435 | JW13166_Direct_Repeats | GAACUAUAGUAGUGUAAAUUUACACUACUCUAAAAC |
| 436 | JW13167_Direct_Repeats | GCUGUUAUAUCCUUACCUUUGUAAGGGAAGUACAGC |
| 437 | JW13168_Direct_Repeats | GUUGUAGUUCCCUUUUAUUUAGGGAUGGUGUUCAGC |
| 438 | JW13169_Direct_Repeats | GUUGUAAAAUCUCUCGUUUUGGAGAGGAUUAACAAC |
| 439 | JW13170_Direct_Repeats | GUUGUAGAAGCCUGUCGUUUGGAUGGGUAU |
| 440 | JW13171_Direct_Repeats | GUUGUCAUACCUACCCAAACGAUAGGCUUCUACAACU |
| 441 | JW13172_Direct_Repeats | CUUAUACAACACCCAUUUUCACAGUGGGUCUAUAAC |
| 442 | JW13173_Direct_Repeats | GUUGUCAUACCGCUCCGAAUGAACGGCUUCUACAAC |
| 443 | JW13174_Direct_Repeats | GUUGUCAUACCGCCCCGAAUGAGCGGCUUCUACAAU |
| 444 | JW13175_Direct_Repeats | GCUGUCAUUACCCUCCAAAACGAGGGCUUCUACAGC |
| 445 | JW13176_Direct_Repeats | GUUUCAGAACCCCGUAAUUCUACGGGGUUGUAGUUG |
| 446 | JW13177_Direct_Repeats | GUUUUAGACCCCUGUGAUUCAGGGGUGUAGUGAAACU |
| 447 | JW13178_Direct_Repeats | GUUUUAAGACUGUGCAAAUUCGCAUAGCUUUCAAUC |
| 448 | JW13179_Direct_Repeats | GUUGUAGAAGCCUGUCGUUUGGAUGGGUAUAACAAC |
| 449 | JW13180_Direct_Repeats | GUUGUCAUACCCACUCAAAUUGUGGGCUUCUACAAC |
| 450 | JW13181_Direct_Repeats | GUUUCCAUACCCACUCAAAUGCGGGCUUCUGAAAC |
| 451 | JW13182_Direct_Repeats | GUUGUGUGUGCCUUUCAAAUUGAAGGCGUUCCCAAC |
| 452 | JW13183_Direct_Repeats | GCUGUACUUACCAUACAAAUUUAUGGCUUUUACAGC |
| 453 | JW13184_Direct_Repeats | GUGCGAGACCAUCCCCAAAUGAAGGGGAACUACACC |
| 454 | JW13185_Direct_Repeats | GGUGUAGUUCCCAUUUAGUUGGGGAUGUUCUCCCGC |
| 455 | JW13186_Direct_Repeats | GGUGCAGUUCCCCUUCAUUUGGGGAUGGUGUUGAGC |
| 456 | JW13187_Direct_Repeats | UGCUGUAACAGCCCUGAUUUUGCAGGGUAAAUACAGUC |
| 457 | JW13188_Direct_Repeats | CAAAAUGGAGACUUCAUCAGC |
| 458 | JW13189_Direct_Repeats | ACCUCUCAAAAUGAGGCCUUCAACAAC |
| 459 | JW13190_Direct_Repeats | GUUGUAGAAGCUUAUCGUUUGGAUAGGUAUGACAAC |
| 460 | JW13191_Direct_Repeats | AAGACUGGUAUAUGAACCAGGGAGACAACA |
| 461 | JW13192_Direct_Repeats | GUUGUCAUGCCGAUCCUAACGACAGGCUUCUACAAC |
| 462 | JW13193_Direct_Repeats | GUUGUAAAGACCUGCACUUUAGUAGGAAGAAACAAC |
| 463 | JW13194_Direct_Repeats | GUUGUAGAAGCCGUUCAUUCGGAGCGGUAUGACAAC |
| 464 | JW13195_Direct_Repeats | GUACUACACCCAUUUUGCAUGGGUCUAAAAC |
| 465 | JW13196_Direct_Repeats | GUUGUCUUUUCCUAACAAAAUAUAGGUUGCUACAAC |
| 466 | JW13197_Direct_Repeats | GUUGUAGAAGCCGCUCAUUCGGGGCGGUAUGACAAC |

1. A reaction mixture comprising:
(a) a nucleic acid-guided CRISPR nuclease (CRISPR nuclease) having at least 85% amino acid homology to any one of SEQ ID NO:62, SEQ ID NO:22, SEQ ID NO:80, SEQ ID NO:91, SEQ ID NOs:1-21, SEQ ID NOs: 23-61, SEQ ID NOs:63-79; SEQ ID NOs:81-90, and SEQ ID NOs:92-93;
(b) a guide nucleic acid (gRNA) for complexing with the nucleic acid-guided CRISPR nuclease, thereby providing a complex comprising a nucleic acid-guided CRISPR nuclease/gRNA pair, wherein the gRNA comprises a direct repeat sequence having at least 85% homology to any one of SEQ ID NOs: 374-466; and
(c) a substrate for the complex optionally, wherein the reaction mixture is in solution, further optionally wherein the solution comprises a buffer that supports optimal pH and ionic conditions for the reaction.

2. (canceled)

3. The reaction mixture of claim 1, wherein the nucleic acid-guided CRISPR nuclease has at least 95% amino acid homology to any one of SEQ ID NOs:1-93.

4. (canceled)

5. The reaction mixture of claim 1, wherein the nucleic acid-guided CRISPR nuclease and the guide nucleic acid comprise a nucleic acid-guided CRISPR nuclease/gRNA pair selected from the group consisting of:
a. the CRISPR nuclease/direct repeat pair of SEQ ID NO:22/SEQ ID NO: 395
b. the CRISPR nuclease/direct repeat pair of SEQ ID NO:62/SEQ ID NO: 435
c. the CRISPR nuclease/direct repeat pair of SEQ ID NO:80/SEQ ID NO: 453
d. the CRISPR nuclease/direct repeat pair of SEQ ID NO:91/SEQ ID NO: 464.

6. The reaction mixture of claim 1, wherein the nucleic acid-guided CRISPR nuclease and the guide nucleic acid comprise a nucleic acid-guided CRISPR nuclease/gRNA pair selected from the group consisting of:
e. the CRISPR nuclease/gRNA pair of SEQ ID NO:1/SEQ ID NO: 94 or the CRISPR nuclease/gRNA pair of SEQ ID NO:1/SEQ ID NO: 117;
f. the CRISPR nuclease/gRNA pair of SEQ ID NO:2/SEQ ID NO: 95 or the CRISPR nuclease/gRNA pair of SEQ ID NO:2/SEQ ID NO: 118;
g. the CRISPR nuclease/gRNA pair of SEQ ID NO:3/SEQ ID NO: 96 or the CRISPR nuclease/gRNA pair of SEQ ID NO:3/SEQ ID NO: 119;
h. the CRISPR nuclease/gRNA pair of SEQ ID NO:4/SEQ ID NO: 97 or the CRISPR nuclease/gRNA pair of SEQ ID NO:4/SEQ ID NO: 120;
i. the CRISPR nuclease/gRNA pair of SEQ ID NO:5/SEQ ID NO: 98 or the CRISPR nuclease/gRNA pair of SEQ ID NO:5/SEQ ID NO: 121;
j. the CRISPR nuclease/gRNA pair of SEQ ID NO:6/SEQ ID NO: 99 or the CRISPR nuclease/gRNA pair of SEQ ID NO:6/SEQ ID NO: 122;
k. the CRISPR nuclease/gRNA pair of SEQ ID NO:7/SEQ ID NO: 100 or the CRISPR nuclease/gRNA pair of SEQ ID NO:7/SEQ ID NO: 123;
l. the CRISPR nuclease/gRNA pair of SEQ ID NO:8/SEQ ID NO: 101 or the CRISPR nuclease/gRNA pair of SEQ ID NO:8/SEQ ID NO: 124;
m. the CRISPR nuclease/gRNA pair of SEQ ID NO:9/SEQ ID NO: 102 or the CRISPR nuclease/gRNA pair of SEQ ID NO:9/SEQ ID NO: 125;
n. the CRISPR nuclease/gRNA pair of SEQ ID NO:10/SEQ ID NO: 103 or the CRISPR nuclease/gRNA pair of SEQ ID NO:10/SEQ ID NO: 126;
o. the CRISPR nuclease/gRNA pair of SEQ ID NO:11/SEQ ID NO: 104 or the CRISPR nuclease/gRNA pair of SEQ ID NO:11/SEQ ID NO: 127;
p. the CRISPR nuclease/gRNA pair of SEQ ID NO:12/SEQ ID NO: 105 or the CRISPR nuclease/gRNA pair of SEQ ID NO:12/SEQ ID NO: 128;
q. the CRISPR nuclease/gRNA pair of SEQ ID NO:13/SEQ ID NO: 106 or the CRISPR nuclease/gRNA pair of SEQ ID NO:13/SEQ ID NO: 129;
r. the CRISPR nuclease/gRNA pair of SEQ ID NO:14/SEQ ID NO: 107 or the CRISPR nuclease/gRNA pair of SEQ ID NO:14/SEQ ID NO: 130;
s. the CRISPR nuclease/gRNA pair of SEQ ID NO:15/SEQ ID NO: 108 or the CRISPR nuclease/gRNA pair of SEQ ID NO:15/SEQ ID NO: 131;
t. the CRISPR nuclease/gRNA pair of SEQ ID NO:16/SEQ ID NO: 109 or the CRISPR nuclease/gRNA pair of SEQ ID NO:16/SEQ ID NO: 132;
u. the CRISPR nuclease/gRNA pair of SEQ ID NO:17/SEQ ID NO: 110 or the CRISPR nuclease/gRNA pair of SEQ ID NO:17/SEQ ID NO: 133;
v. the CRISPR nuclease/gRNA pair of SEQ ID NO:18/SEQ ID NO: 111 or the CRISPR nuclease/gRNA pair of SEQ ID NO:18/SEQ ID NO: 134;
w. the CRISPR nuclease/gRNA pair of SEQ ID NO:19/SEQ ID NO: 112 or the CRISPR nuclease/gRNA pair of SEQ ID NO:19/SEQ ID NO: 135;
x. the CRISPR nuclease/gRNA pair of SEQ ID NO:20/SEQ ID NO: 113 or the CRISPR nuclease/gRNA pair of SEQ ID NO:20/SEQ ID NO: 136;
y. the CRISPR nuclease/gRNA pair of SEQ ID NO:21/SEQ ID NO: 114 or the CRISPR nuclease/gRNA pair of SEQ ID NO:21/SEQ ID NO: 137;
z. the CRISPR nuclease/gRNA pair of SEQ ID NO:22/SEQ ID NO: 115 or the CRISPR nuclease/gRNA pair of SEQ ID NO:22/SEQ ID NO: 138;
aa. the CRISPR nuclease/gRNA pair of SEQ ID NO:23/SEQ ID NO: 116 or the CRISPR nuclease/gRNA pair of SEQ ID NO:23/SEQ ID NO: 139;
bb. the CRISPR nuclease/gRNA pair of SEQ ID NO:24/SEQ ID NO: 140 or the CRISPR nuclease/gRNA pair of SEQ ID NO:24/SEQ ID NO: 164;
cc. the CRISPR nuclease/gRNA pair of SEQ ID NO:25/SEQ ID NO: 141 or the CRISPR nuclease/gRNA pair of SEQ ID NO:25/SEQ ID NO: 165;
dd. the CRISPR nuclease/gRNA pair of SEQ ID NO:26/SEQ ID NO: 142 or the CRISPR nuclease/gRNA pair of SEQ ID NO:26/SEQ ID NO: 166;
ee. the CRISPR nuclease/gRNA pair of SEQ ID NO:27/SEQ ID NO: 143 or the CRISPR nuclease/gRNA pair of SEQ ID NO:27/SEQ ID NO: 167;
ff. the CRISPR nuclease/gRNA pair of SEQ ID NO:28/SEQ ID NO: 144 or the CRISPR nuclease/gRNA pair of SEQ ID NO:28/SEQ ID NO: 168;
gg. the CRISPR nuclease/gRNA pair of SEQ ID NO:29/SEQ ID NO: 145 or the CRISPR nuclease/gRNA pair of SEQ ID NO:29/SEQ ID NO: 169;
hh. the CRISPR nuclease/gRNA pair of SEQ ID NO:30/SEQ ID NO: 146 or the CRISPR nuclease/gRNA pair of SEQ ID NO:30/SEQ ID NO: 170;
ii. the CRISPR nuclease/gRNA pair of SEQ ID NO:31/SEQ ID NO: 147 or the CRISPR nuclease/gRNA pair of SEQ ID NO:31/SEQ ID NO: 171;
jj. the CRISPR nuclease/gRNA pair of SEQ ID NO:32/SEQ ID NO: 148 or the CRISPR nuclease/gRNA pair of SEQ ID NO:32/SEQ ID NO: 172;
kk. the CRISPR nuclease/gRNA pair of SEQ ID NO:33/SEQ ID NO: 149 or the CRISPR nuclease/gRNA pair of SEQ ID NO:33/SEQ ID NO: 173;
ll. the CRISPR nuclease/gRNA pair of SEQ ID NO:34/SEQ ID NO: 150 or the CRISPR nuclease/gRNA pair of SEQ ID NO:34/SEQ ID NO: 174;
mm. the CRISPR nuclease/gRNA pair of SEQ ID NO:35/SEQ ID NO: 151 or the CRISPR nuclease/gRNA pair of SEQ ID NO:35/SEQ ID NO: 175;
nn. the CRISPR nuclease/gRNA pair of SEQ ID NO:36/SEQ ID NO: 152 or the CRISPR nuclease/gRNA pair of SEQ ID NO:36/SEQ ID NO: 176;
oo. the CRISPR nuclease/gRNA pair of SEQ ID NO:37/SEQ ID NO: 153 or the CRISPR nuclease/gRNA pair of SEQ ID NO:37/SEQ ID NO: 177;
pp. the CRISPR nuclease/gRNA pair of SEQ ID NO:38/SEQ ID NO: 154 or the CRISPR nuclease/gRNA pair of SEQ ID NO:38/SEQ ID NO: 178;
qq. the CRISPR nuclease/gRNA pair of SEQ ID NO:39/SEQ ID NO: 155 or the CRISPR nuclease/gRNA pair of SEQ ID NO:39/SEQ ID NO: 179;
rr. the CRISPR nuclease/gRNA pair of SEQ ID NO:40/SEQ ID NO: 156 or the CRISPR nuclease/gRNA pair of SEQ ID NO:40/SEQ ID NO: 180;
ss. the CRISPR nuclease/gRNA pair of SEQ ID NO:41/SEQ ID NO: 157 or the CRISPR nuclease/gRNA pair of SEQ ID NO:41/SEQ ID NO: 181;
tt. the CRISPR nuclease/gRNA pair of SEQ ID NO:42/SEQ ID NO: 158 or the CRISPR nuclease/gRNA pair of SEQ ID NO:42/SEQ ID NO: 182;
uu. the CRISPR nuclease/gRNA pair of SEQ ID NO:43/SEQ ID NO: 159 or the CRISPR nuclease/gRNA pair of SEQ ID NO:43/SEQ ID NO: 183;
vv. the CRISPR nuclease/gRNA pair of SEQ ID NO:44/SEQ ID NO: 160 or the CRISPR nuclease/gRNA pair of SEQ ID NO:44/SEQ ID NO: 184;
ww. the CRISPR nuclease/gRNA pair of SEQ ID NO:45/SEQ ID NO: 161 or the CRISPR nuclease/gRNA pair of SEQ ID NO:45/SEQ ID NO: 185;
xx. the CRISPR nuclease/gRNA pair of SEQ ID NO:46/SEQ ID NO: 162 or the CRISPR nuclease/gRNA pair of SEQ ID NO:46/SEQ ID NO: 186;
yy. the CRISPR nuclease/gRNA pair of SEQ ID NO:47/SEQ ID NO: 163 or the CRISPR nuclease/gRNA pair of SEQ ID NO:47/SEQ ID NO: 187;
zz. the CRISPR nuclease/gRNA pair of SEQ ID NO:48/SEQ ID NO: 188 or the CRISPR nuclease/gRNA pair of SEQ ID NO:48/SEQ ID NO: 234;
aaa. the CRISPR nuclease/gRNA pair of SEQ ID NO:49/SEQ ID NO: 189 or the CRISPR nuclease/gRNA pair of SEQ ID NO:49/SEQ ID NO: 235;
bbb. the CRISPR nuclease/gRNA pair of SEQ ID NO:50/SEQ ID NO: 190 or the CRISPR nuclease/gRNA pair of SEQ ID NO:50/SEQ ID NO: 236;
ccc. the CRISPR nuclease/gRNA pair of SEQ ID NO:51/SEQ ID NO: 191 or the CRISPR nuclease/gRNA pair of SEQ ID NO:51/SEQ ID NO: 237;
ddd. the CRISPR nuclease/gRNA pair of SEQ ID NO:52/SEQ ID NO: 192 or the CRISPR nuclease/gRNA pair of SEQ ID NO:52/SEQ ID NO: 238;
eee. the CRISPR nuclease/gRNA pair of SEQ ID NO:53/SEQ ID NO: 193 or the CRISPR nuclease/gRNA pair of SEQ ID NO:53/SEQ ID NO: 239;
fff. the CRISPR nuclease/gRNA pair of SEQ ID NO:54/SEQ ID NO: 194 or the CRISPR nuclease/gRNA pair of SEQ ID NO:54/SEQ ID NO: 240;
ggg. the CRISPR nuclease/gRNA pair of SEQ ID NO:55/SEQ ID NO: 195 or the CRISPR nuclease/gRNA pair of SEQ ID NO:55/SEQ ID NO: 241;
hhh. the CRISPR nuclease/gRNA pair of SEQ ID NO:56/SEQ ID NO: 196 or the CRISPR nuclease/gRNA pair of SEQ ID NO:56/SEQ ID NO: 242;
iii. the CRISPR nuclease/gRNA pair of SEQ ID NO:57/SEQ ID NO: 197 or the CRISPR nuclease/gRNA pair of SEQ ID NO:57/SEQ ID NO: 243;
jjj. the CRISPR nuclease/gRNA pair of SEQ ID NO:58/SEQ ID NO: 198 or the CRISPR nuclease/gRNA pair of SEQ ID NO:58/SEQ ID NO: 244;
kkk. the CRISPR nuclease/gRNA pair of SEQ ID NO:59/SEQ ID NO: 199 or the CRISPR nuclease/gRNA pair of SEQ ID NO:59/SEQ ID NO: 245;
lll. the CRISPR nuclease/gRNA pair of SEQ ID NO:60/SEQ ID NO: 200 or the CRISPR nuclease/gRNA pair of SEQ ID NO:60/SEQ ID NO: 246;
mmm. the CRISPR nuclease/gRNA pair of SEQ ID NO:61/SEQ ID NO: 201 or the CRISPR nuclease/gRNA pair of SEQ ID NO:61/SEQ ID NO: 247;
nnn. the CRISPR nuclease/gRNA pair of SEQ ID NO:62/SEQ ID NO: 202 or the CRISPR nuclease/gRNA pair of SEQ ID NO:62/SEQ ID NO: 248;
ooo. the CRISPR nuclease/gRNA pair of SEQ ID NO:63/SEQ ID NO: 203 or the CRISPR nuclease/gRNA pair of SEQ ID NO:63/SEQ ID NO: 249;
ppp. the CRISPR nuclease/gRNA pair of SEQ ID NO:64/SEQ ID NO: 204 or the CRISPR nuclease/gRNA pair of SEQ ID NO:64/SEQ ID NO: 250;
qqq. the CRISPR nuclease/gRNA pair of SEQ ID NO:65/SEQ ID NO: 205 or the CRISPR nuclease/gRNA pair of SEQ ID NO:65/SEQ ID NO: 251;
rrr. the CRISPR nuclease/gRNA pair of SEQ ID NO:66/SEQ ID NO: 206 or the CRISPR nuclease/gRNA pair of SEQ ID NO:66/SEQ ID NO: 252;
sss. the CRISPR nuclease/gRNA pair of SEQ ID NO:67/SEQ ID NO: 207 or the CRISPR nuclease/gRNA pair of SEQ ID NO:67/SEQ ID NO: 253;
ttt. the CRISPR nuclease/gRNA pair of SEQ ID NO:68/SEQ ID NO: 208 or the CRISPR nuclease/gRNA pair of SEQ ID NO:68/SEQ ID NO: 254;
uuu. the CRISPR nuclease/gRNA pair of SEQ ID NO:69/SEQ ID NO: 209 or the CRISPR nuclease/gRNA pair of SEQ ID NO:69/SEQ ID NO: 255;
vvv. the CRISPR nuclease/gRNA pair of SEQ ID NO:70/SEQ ID NO: 210 or the CRISPR nuclease/gRNA pair of SEQ ID NO:70/SEQ ID NO: 256;
www. the CRISPR nuclease/gRNA pair of SEQ ID NO:71/SEQ ID NO: 211 or the CRISPR nuclease/gRNA pair of SEQ ID NO:71/SEQ ID NO: 257;
xxx. the CRISPR nuclease/gRNA pair of SEQ ID NO:72/SEQ ID NO: 212 or the CRISPR nuclease/gRNA pair of SEQ ID NO:72/SEQ ID NO: 258;
yyy. the CRISPR nuclease/gRNA pair of SEQ ID NO:73/SEQ ID NO: 213 or the CRISPR nuclease/gRNA pair of SEQ ID NO:73/SEQ ID NO: 259;
zzz. the CRISPR nuclease/gRNA pair of SEQ ID NO:74/SEQ ID NO: 214 or the CRISPR nuclease/gRNA pair of SEQ ID NO:74/SEQ ID NO: 260;
aaaa. the CRISPR nuclease/gRNA pair of SEQ ID NO:75/SEQ ID NO: 215 or the CRISPR nuclease/gRNA pair of SEQ ID NO:75/SEQ ID NO: 261;
bbbb. the CRISPR nuclease/gRNA pair of SEQ ID NO:76/SEQ ID NO: 216 or the CRISPR nuclease/gRNA pair of SEQ ID NO:76/SEQ ID NO: 262;
cccc. the CRISPR nuclease/gRNA pair of SEQ ID NO:77/SEQ ID NO: 217 or the CRISPR nuclease/gRNA pair of SEQ ID NO:77/SEQ ID NO: 263;
dddd. the CRISPR nuclease/gRNA pair of SEQ ID NO:78/SEQ ID NO: 218 or the CRISPR nuclease/gRNA pair of SEQ ID NO:78/SEQ ID NO: 264;
eeee. the CRISPR nuclease/gRNA pair of SEQ ID NO:79/SEQ ID NO: 219 or the CRISPR nuclease/gRNA pair of SEQ ID NO:79/SEQ ID NO: 265;
ffff. the CRISPR nuclease/gRNA pair of SEQ ID NO:80/SEQ ID NO: 220 or the CRISPR nuclease/gRNA pair of SEQ ID NO:80/SEQ ID NO: 266;
gggg. the CRISPR nuclease/gRNA pair of SEQ ID NO:81/SEQ ID NO: 221 or the CRISPR nuclease/gRNA pair of SEQ ID NO:81/SEQ ID NO: 267;
hhhh. the CRISPR nuclease/gRNA pair of SEQ ID NO:82/SEQ ID NO: 222 or the CRISPR nuclease/gRNA pair of SEQ ID NO:82/SEQ ID NO: 268;
iiii. the CRISPR nuclease/gRNA pair of SEQ ID NO:83/SEQ ID NO: 223 or the CRISPR nuclease/gRNA pair of SEQ ID NO:83/SEQ ID NO: 269;
jjjj. the CRISPR nuclease/gRNA pair of SEQ ID NO:84/SEQ ID NO: 224 or the CRISPR nuclease/gRNA pair of SEQ ID NO:84/SEQ ID NO: 270;
kkkk. the CRISPR nuclease/gRNA pair of SEQ ID NO:85/SEQ ID NO: 225 or the CRISPR nuclease/gRNA pair of SEQ ID NO:85/SEQ ID NO: 271;
llll. the CRISPR nuclease/gRNA pair of SEQ ID NO:86/SEQ ID NO: 226 or the CRISPR nuclease/gRNA pair of SEQ ID NO:86/SEQ ID NO: 272;
mmmm. the CRISPR nuclease/gRNA pair of SEQ ID NO:87/SEQ ID NO: 227 or the CRISPR nuclease/gRNA pair of SEQ ID NO:87/SEQ ID NO: 273;
nnnn. the CRISPR nuclease/gRNA pair of SEQ ID NO:88/SEQ ID NO: 228 or the CRISPR nuclease/gRNA pair of SEQ ID NO:88/SEQ ID NO: 274;
oooo. the CRISPR nuclease/gRNA pair of SEQ ID NO:89/SEQ ID NO: 229 or the CRISPR nuclease/gRNA pair of SEQ ID NO:89/SEQ ID NO: 275;
pppp. the CRISPR nuclease/gRNA pair of SEQ ID NO:90/SEQ ID NO: 230 or the CRISPR nuclease/gRNA pair of SEQ ID NO:90/SEQ ID NO: 276;
qqqq. the CRISPR nuclease/gRNA pair of SEQ ID NO:91/SEQ ID NO: 231 or the CRISPR nuclease/gRNA pair of SEQ ID NO:91/SEQ ID NO: 277;
rrrr. the CRISPR nuclease/gRNA pair of SEQ ID NO:92/SEQ ID NO: 232 or the CRISPR nuclease/gRNA pair of SEQ ID NO:92/SEQ ID NO: 278;
ssss. the CRISPR nuclease/gRNA pair of SEQ ID NO:93/SEQ ID NO: 233 or the CRISPR nuclease/gRNA pair of SEQ ID NO:93/SEQ ID NO: 279.

7. The reaction mixture of claim 6, wherein the CRISPR nuclease/gRNA pair is SEQ ID NO:22/SEQ ID NO: 115 or the CRISPR nuclease/gRNA pair of SEQ ID NO:22/SEQ ID NO: 138.

8. The reaction mixture of claim 1, wherein the nucleic acid-guided CRISPR nuclease has trans-cleavage activity.

9. The reaction mixture of claim 1, wherein the nucleic acid-guided CRISPR nuclease hybridizes to a PAM region in a target nucleic acid sequence.

10. The reaction mixture of claim 1, wherein the substrate is a ribonucleic acid molecule (RNA) complexed with a fluorophore and a quencher for the fluorophore.

11. The reaction mixture of claim 10, wherein the reaction mixture is in solution, and wherein the substrate is present in the reaction mix in a concentration ranging from 250 nM to 450 nM.

12. The reaction mixture of claim 1, wherein the substrate is a ribonucleic acid molecule (RNA) complexed with biotin.

13. The reaction mixture of claim 1, wherein the substrate is a deoxyribonucleic acid (DNA) complexed with a fluorophore and a quencher for the fluorophore.

14. The reaction mixture of claim 1, wherein the reaction mixture further comprises a target nucleic acid sequence.

15. The reaction mixture of claim 14, wherein the target nucleic acid sequence is a sequence from an influenza A virus, an influenza B virus, a Respiratory Syncytial Virus (RSV), or a SARS-CoV-2 virus.

16. The reaction mixture of claim 15, wherein the target nucleic acid sequence is from an influenza A virus.

17. The reaction mixture of claim 16, wherein the target nucleic acid sequence is SEQ ID NO:373.

18. The reaction mixture of claim 15, wherein the target nucleic acid sequence activates cleavage of the substrate by the nucleic acid-guided CRISPR nuclease.

19.-42. (canceled)

43. A nucleic acid comprising a sequence having at least 95% identical to any one of SEQ ID NO:341, SEQ ID NO:301, SEQ ID NO:359, SEQ ID NO:370, SEQ ID NOs:280-300, SEQ ID NOs:302-340, SEQ ID NOs:342-358, SEQ ID NOs:360-369, and SEQ ID NOs:371-372, wherein the nucleic acid sequence encodes a nucleic acid-guided CRISPR nuclease comprising an amino acid sequence of any one of SEQ ID NOs:1-93 and, optionally further comprising a heterologous sequence optionally configured to facilitate the recombinant expression of the nucleic acid-guided CRISPR nuclease in an organism.

44.-45. (canceled)

46. An enzyme system comprising:
(a) a nucleic acid-guided CRISPR nuclease having at least 85% amino acid homology to any one of SEQ ID NO:62, SEQ ID NO:22, SEQ ID NO:80, SEQ ID NO:91, SEQ ID NOs:1-21, SEQ ID NOs:23-61, SEQ ID NOs:63-79, SEQ ID NOs:81-90, and SEQ ID NOs:92-93; and
(b) a guide nucleic acid (gRNA) for complexing with the nucleic acid-guided CRISPR nuclease, wherein the gRNA comprises a direct repeat sequence having at least 85% homology to any one of SEQ ID NOs: 374-466.

47.-66. (canceled)

67. A method of detecting a target nucleic acid, the method comprising:
(a) contacting a sample with a nucleic acid-guided nuclease system comprising:
a nucleic acid-guided CRISPR nuclease having at least 90% identity to SEQ ID NO:62, SEQ ID NO:22, SEQ ID NO:80, SEQ ID NO:91, SEQ ID NO:1-21, SEQ ID NOs: 23-61, SEQ ID NOs:63-79; SEQ ID NOs:81-90, and SEQ ID NOs:92-93, or a nucleic acid molecule encoding the nucleic acid-guided CRISPR nuclease;
a guide nucleic acid (gRNA) for complexing with the nucleic acid-guided CRISPR nuclease, or a nucleic acid molecule encoding the guide nucleic acid,
wherein the gRNA:
(i) comprises a direct repeat sequence having at least 65%, optionally at least 90% homology to any one of SEQ ID NOs: 374-466, optionally wherein the direct repeat sequence has (a) at least 65%, at least 85%, at least 90%, or at least 95% homology to a direct repeat sequence selected from the group consisting of: SEQ ID NO: 395, SEQ ID NO: 435, SEQ ID NO: 453; SEQ ID NO: 464 or (b) a sequence selected from the group consisting of: SEQ ID NO: 395; SEQ ID NO: 435: SEQ ID NO: 453; or SEQ ID NO: 464;
(ii) comprises a nucleic acid sequence having at least 85%, at least 90%, or at least 95% homology to any one of SEQ ID NO: 115, SEQ ID NO: 202, SEQ ID NO:220, or SEQ ID NO: 138 or (ii) a nucleic acid sequence selected from the group consisting of the sequence set forth as SEQ ID NO: 115, SEQ ID NO: 202, SEQ ID NO: 220, or SEQ ID NO: 138, and
(b) allowing the target nucleic acid to activate the nucleic acid-guided CRISPR nuclease system to cleave a detectable substrate.

68. The method of claim 67, wherein the direct repeat sequence of the gRNA comprises a direct repeat sequence having at least 65%, at least 85%, at least 90%, or at least 95% homology to SEQ ID NO: 395; SEQ ID NO:435; SEQ ID NO:453; or SEQ ID NO:464.

69. The method of claim 67, wherein the nucleic acid-guided nuclease system is an enzyme system of claim 46.

70. The method of claim 67, further comprising detecting the presence of the target nucleic acid by detecting the cleavable substrate cleaved by the nucleic-acid guided CRISPR nuclease system.

71. The method of claim 67, wherein the nuclease is JW13166 (SEQ ID NO:62), and optionally
wherein the guide nucleic acid is or comprises a nucleic acid sequence having at least 85%, at least 90%, or at least 95% homology to SEQ ID NO: 202 and/or SEQ ID NO: 248.
